(19) United States
(12) Patent Application Publication
(10) Pub. No.: US 2023/0219996 A1
(43) Pub. Date: Jul. 13, 2023

(54) RNA VACCINES

(71) Applicant: Arcturus Therapeutics, Inc., San Diego, CA (US)

(72) Inventors: Daiki MATSUDA, San Diego, CA (US); Sean Michael Sullivan, Carlsbad, CA (US); Kiyoshi Tachikawa, San Diego, CA (US); Padmanabh Chivukula, San Diego, CA (US); Priya Prakash Karmali, San Diego, CA (US); Yanjie Bao, San Diego, CA (US); Amit Sagi, San Diego, CA (US); Rajesh Mukthavaram, San Diego, CA (US)

(21) Appl. No.: 17/816,243

(22) Filed: Jul. 29, 2022

Related U.S. Application Data
(60) Provisional application No. 63/227,972, filed on Jul. 30, 2021.

(51) Int. Cl. C07H 21/02; C07K 14/18; C07K 14/165; C07K 14/11; A61K 9/127; A61K 47/14; A61K 47/28; A61K 9/19

(52) U.S. Cl. CPC C07H 21/02; A61K 9/19; A61K 9/127; A61K 47/14; A61K 47/28; C07K 14/11; C07K 14/165; C07K 14/1808

(57) ABSTRACT

Provided herein are RNA molecules encoding viral replication proteins and antigenic proteins or fragments thereof. Also provided herein are compositions that include RNA molecules encoding viral replication proteins and antigenic proteins or fragments thereof, and lipids. RNA molecules and compositions including them are useful for inducing immune responses.

### CROSS-REFERENCES TO RELATED APPLICATIONS

This application claims the benefit of U.S. Provisional Application No. 63/227,972, filed Jul. 30, 2021, which is incorporated herein by reference in its entirety and for all purposes.

### SEQUENCE LISTING

The instant application contains a Sequence Listing, which has been submitted electronically in ASCII format and is hereby incorporated by reference in its entirety. Said ASCII copy, created on Jul. 22, 2022 is named “049386-544001WO_SL_ST26.xml” and is 485,649 bytes in size.

### TECHNICAL FIELD

The present disclosure relates generally to inducing immune responses against infectious agents and more specifically to RNA molecules and liponanoparticles as vaccines.

### BACKGROUND

Infectious diseases represent significant burdens on health worldwide. According to the World Health Organization (WHO), lower respiratory tract infection was the deadliest infectious disease worldwide in 2016, causing approximately 3 million deaths. The impact of infectious diseases is illustrated by the coronavirus disease 2019 (COVID-19) pandemic caused by severe acute respiratory syndrome-coronavirus-2 (SARS-CoV-2). SARS-CoV-2 is a novel coronavirus that was first identified in December 2019 in Wuhan, China and that has caused more than 184 million confirmed infections and nearly 4 million deaths worldwide as of July 2021. Control measures to curb the rapid worldwide spread of SARS-CoV-2, such as national lockdowns, closure of workplaces and schools, and reduction of international travel have been damaging to global economies and social wellbeing.

Self-replicating ribonucleic acids (RNAs), e.g., RNAs derived from viral replicons, and messenger RNAs (mRNAs) are useful for expression of proteins, such as heterologous proteins, for a variety of purposes, such as expression of therapeutic proteins and expression of antigens for vaccines. A desirable property of replicons is the ability for sustained expression of the protein.

Few treatments for infections caused by viruses and eukaryotic organisms are available, and resistance to antibiotics for the treatment of bacterial infections is increasing. In addition, rapid responses, including rapid vaccine development, are required to effectively control emerging infectious diseases and pandemics. Thus, there exists a need for the prevention and/or treatment of infectious diseases and cancer.

### SUMMARY

The present disclosure provides RNA molecules that are useful for inducing immune responses. Both self-replicating RNA molecules and messenger RNA (mRNA) molecules are provided.

In some embodiments, provided herein are RNA molecules comprising: (a) a first polynucleotide encoding one or more viral replication proteins, wherein one or more miRNA binding sites in the first polynucleotide have been modified as compared to a reference polynucleotide; and (b) a second polynucleotide comprising a first transgene encoding a first antigenic protein or a fragment thereof.

Also provided herein, in some embodiments, are RNA molecules comprising: (i) a first polynucleotide comprising a sequence having at least 80% identity to a sequence of SEQ ID NO:6; and (ii) a second polynucleotide comprising a first transgene encoding a first antigenic protein or a fragment thereof.

In some aspects, modification of the one or more miRNA binding sites reduces or eliminates miRNA binding. In some aspects, two, three, four, five, six, seven, eight, nine, ten, 11, 12, 13, 14, or 15 miRNA binding sites in the first polynucleotide have been modified. In some aspects, the one or more miRNA binding sites are selected from regions that bind a miRNA having a sequence of SEQ ID NOs:58, 59, 72, 80, 81, 83, 101, 102, 103, 112, 113, 114, 128, 131, 142, 156, 157, 171, 175, and any combination thereof.

In some aspects, the one or more viral replication proteins of RNA molecules provided herein are alphavirus proteins or rubivirus proteins. In some aspects, the alphavirus proteins are from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O’Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), Buggy Creek Virus (BCRV), or any combination thereof.

In some aspects, first polynucleotides of RNA molecules provided herein encode a polyprotein comprising an alphavirus nsP1 protein, an alphavirus nsP2 protein, an alphavirus nsP3 protein, an alphavirus nsP4 protein, or any combination thereof. In some aspects, first polynucleotides encode a polyprotein comprising an alphavirus nsP1 protein, an alphavirus nsP2 protein, an alphavirus nsP3 protein, or any combination thereof, and an alphavirus nsP4 protein. In some aspects, first polynucleotides comprise a sequence having at least 80% identity to a sequence of SEQ ID NO:6. In some aspects, first polynucleotides comprise a sequence having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to a sequence of SEQ ID NO:6. In some aspects, first polynucleotides encode a polyprotein comprising a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to a sequence of SEQ ID NO:187.

In some aspects, RNA molecules provided herein include a 5′ untranslated region (UTR). In some aspects, the 5′ UTR comprises a viral 5′ UTR, a non-viral 5′ UTR, or a combination of viral and non-viral 5′ UTR sequences. In some aspects, the 5′ UTR comprises an alphavirus 5′ UTR. In some aspects, the alphavirus 5′ UTR comprises a Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O’Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV) 5′ UTR sequence. In some aspects, the 5′ UTR comprises a sequence of SEQ ID NO:5.

In some aspects, RNA molecules provided herein include a 3′ untranslated region (UTR). In some aspects, the 3′ UTR comprises a viral 3′ UTR, a non-viral 3′ UTR, or a combination of viral and non-viral 3′ UTR sequences. In some aspects, the 3′ UTR comprises an alphavirus 3′ UTR.\ In some aspects, the alphavirus 3′ UTR comprises a Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O’Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV) 3′ UTR sequence. In some aspects, the 3′ UTR comprises a sequence of SEQ ID NO:9. In some aspects, the 3′ UTR further comprises a poly-A sequence.

In some aspects, the first antigenic protein of RNA molecules provided herein is a viral protein, a bacterial protein, a fungal protein, a protozoan protein, or a parasite protein. In some aspects, the viral protein is a coronavirus protein, an orthomyxovirus protein, a paramyxovirus protein, a picornavirus protein, a flavivirus protein, a filovirus protein, a rhabdovirus protein, a togavirus protein, an arterivirus protein, a bunyavirus protein, an arenavirus protein, a reovirus protein, a bornavirus protein, a retrovirus protein, an adenovirus protein, a herpesvirus protein, a polyomavirus protein, a papillomavirus protein, a poxvirus protein, or a hepadnavirus protein. In some aspects, the first antigenic protein is a SARS-CoV-2 protein, an influenza virus protein, a respiratory syncytial virus (RSV) protein, a human immunodeficiency virus (HIV) protein, a hepatitis C virus (HCV) protein, a cytomegalovirus (CMV) protein, a Lassa Fever Virus (LFV) protein, an Ebola Virus (EBOV) protein, a Mycobacterium protein, a Bacillus protein, a Yersinia protein, a Streptococcus protein, a Pseudomonas protein, a Shigella protein, a Campylobacter protein, a Salmonella protein, a Plasmodium protein, or a Toxoplasma protein. In some aspects, the first antigenic protein is a SARS-CoV-2 spike glycoprotein. In some aspects, the SARS-CoV-2 spike glycoprotein comprises an amino acid sequence having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to a sequence of SEQ ID NO: 14, SEQ ID NO: 15, SEQ ID NO: 16, or SEQ ID NO: 17. In some aspects, the second polynucleotide of RNA molecules provided herein comprises a sequence having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to a sequence of SEQ ID NO: 10, SEQ ID NO: 11, SEQ ID NO: 12, or SEQ ID NO:13. In some aspects, the first transgene of RNA molecules provided herein is expressed from a first subgenomic promoter.

In some aspects, the second polynucleotide of RNA molecules provided herein includes at least two transgenes. In some aspects, a second transgene of the second polynucleotide encodes a second antigenic protein or a fragment thereof or an immunomodulatory protein. In some aspects, the second polynucleotide further comprises a sequence encoding a 2A peptide, an internal ribosomal entry site (IRES), a second subgenomic promoter, or a combination thereof, located between transgenes. In some aspects, the immunomodulatory protein is a cytokine, a chemokine, or an interleukin. In some aspects, first and second transgenes of second polynucleotides encode viral proteins, bacterial proteins, fungal proteins, protozoan proteins, parasite proteins, immunomodulatory proteins, or any combination thereof.

In some aspects, the first polynucleotide is located 5′ of the second polynucleotide. In some aspects, RNA molecules provided herein further include an intergenic region located between the first polynucleotide and the second polynucleotide. In some aspects, the intergenic region comprises a sequence having at least 85% identity to a sequence of SEQ ID NO:7.

In some aspects, RNA molecules provided herein are self-replicating RNA molecules. In some aspects, RNA molecules provided herein include a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to a sequence of SEQ ID NO:1, SEQ ID NO:2, SEQ ID NO:3, or SEQ ID NO:4. In some aspects, RNA molecules provided herein are self-replicating RNA molecules. In some aspects, RNA molecules provided herein include a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to a sequence of SEQ ID NO:29, SEQ ID NO:32, SEQ ID NO:40, or SEQ ID NO:48.

In some aspects, RNA molecules provided herein further include a 5′ cap. In some aspects, the 5′ cap has a Cap 1 structure, a Cap 1 (^{m6}A) structure, a Cap 2 structure, or a Cap 0 structure.

Provided herein, in some embodiments, are DNA molecules encoding any of the RNA molecules provided herein. In some aspects, DNA molecules provided herein include a promoter. In some aspects, the promoter is located 5′ of the 5 ’UTR. In some aspects, the promoter is a T7 promoter, a T3 promoter, or an SP6 promoter.

Provided herein, in some embodiments, are compositions comprising any RNA molecule provided herein and a lipid. In some aspects, the lipid comprises an ionizable cationic lipid. In some aspects, the ionizable cationic lipid has a structure of

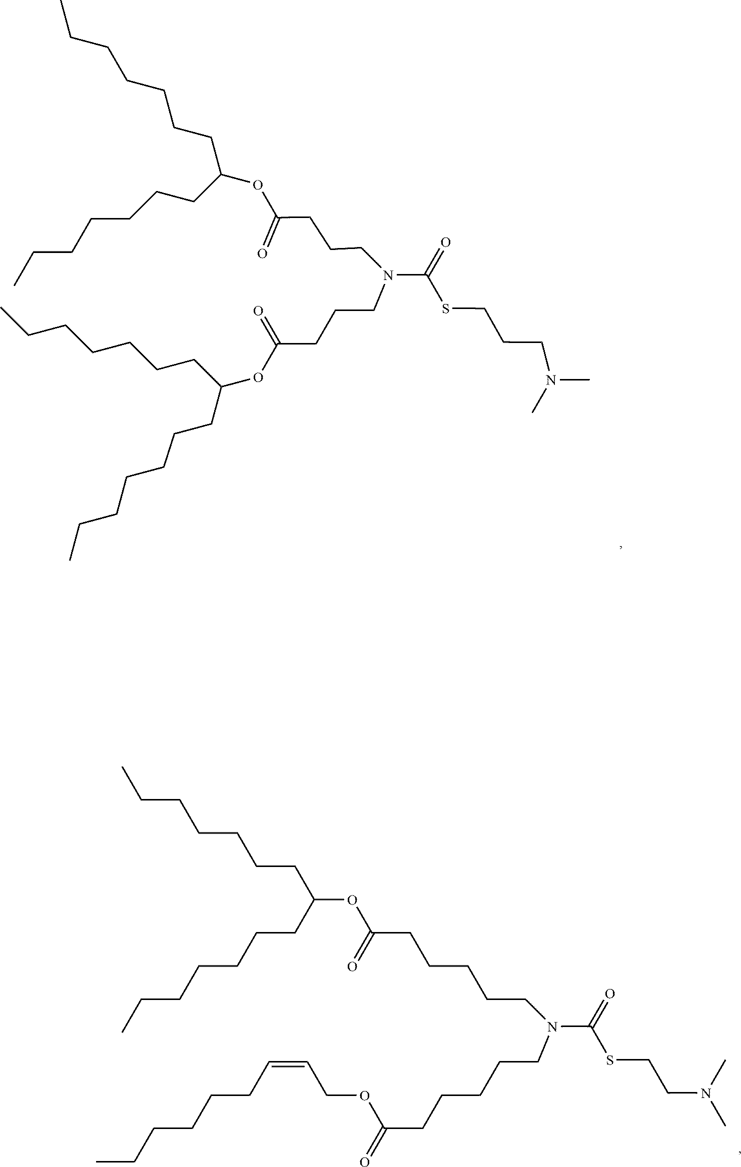

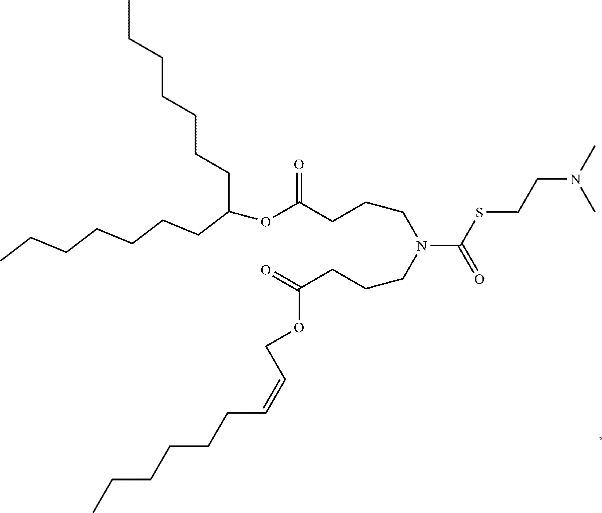

or a pharmaceutically acceptable salt thereof.

Provided herein, in some embodiments, are compositions comprising any RNA molecule provided herein and a lipid formulation.

In some aspects, the lipid formulation comprises an ionizable cationic lipid. In some aspects, the ionizable cationic lipid has a structure of

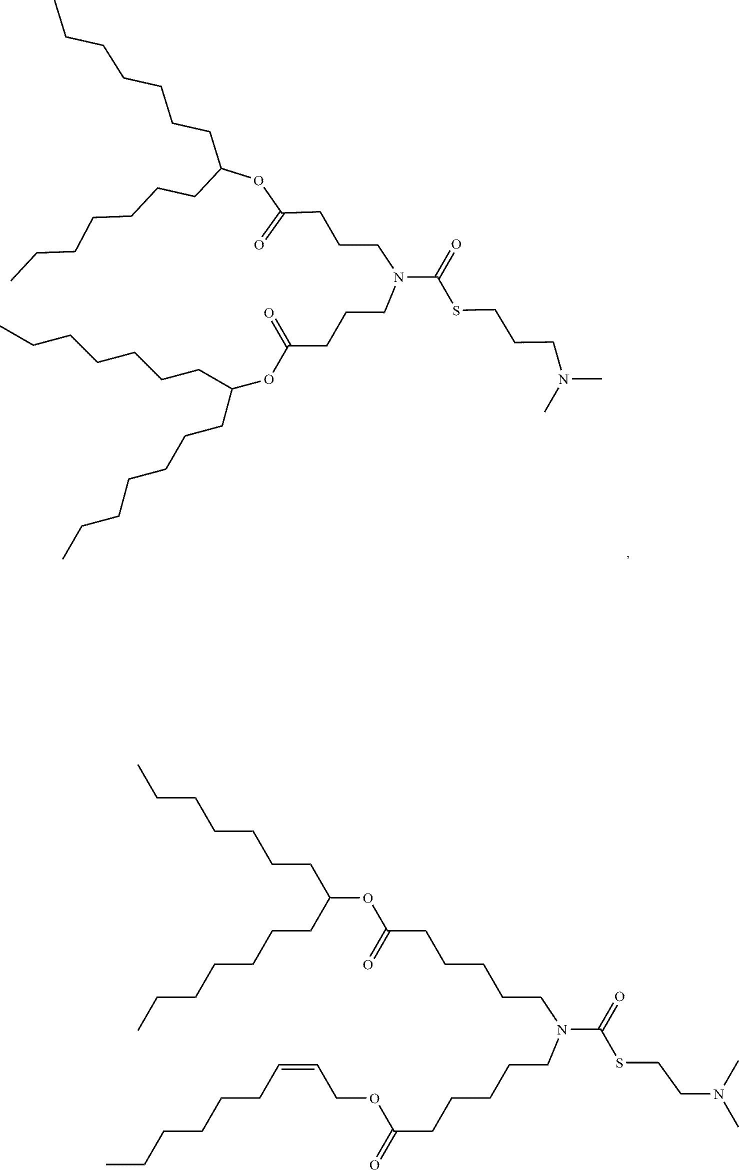

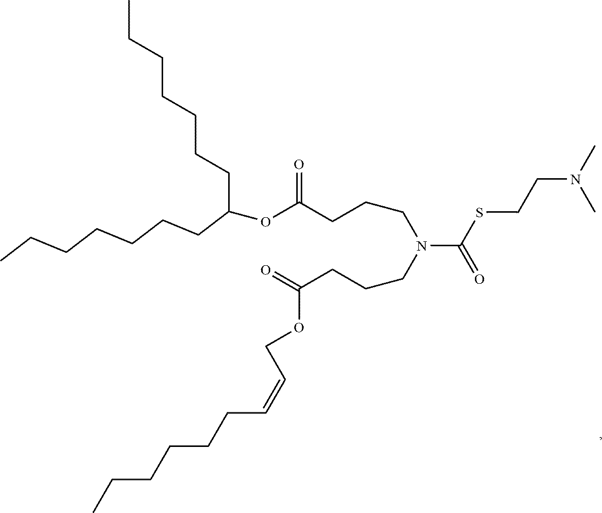

or a pharmaceutically acceptable salt thereof.

In some aspects, the lipid formulation is selected from a lipoplex, a liposome, a lipid nanoparticle, a polymer-based carrier, an exosome, a lamellar body, a micelle, and an emulsion. In some aspects, the lipid formulation is a liposome selected from a cationic liposome, a nanoliposome, a proteoliposome, a unilamellar liposome, a multilamellar liposome, a ceramide-containing nanoliposome, and a multivesicular liposome. In some aspects, the lipid formulation is a lipid nanoparticle. In some aspects, the lipid nanoparticle has a size of less than about 200 nm. In some aspects, the lipid nanoparticle has a size of less than about 150 nm. In some aspects, the lipid nanoparticle has a size of less than about 100 nm. In some aspects, the lipid nanoparticle has a size of about 55 nm to about 90 nm. In some aspects, the lipid formulation comprises one or more cationic lipids. In some aspects, the one or more cationic lipids is selected from 5-carboxyspermylglycinedioctadecylamide (DOGS), 2,3-dioleyloxy-N-[2(spermine-carboxamido)ethyl]-N,N-dimethyl-l-propanaminium (DOSPA), 1,2-Dioleoyl-3-Dimethylammonium-Propane (DODAP), 1,2-Dioleoyl-3-Trimethylammonium-Propane (DOTAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-difinolenyloxy-N,N-dimethyl-3-aminopropane (DLenDMA), N-dioleyl-N,N-dimethylammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethy 1-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[l,3]-dioxolane (DLin-K-DMA), and 2,2-dilinoleyl-4-dimethylaminoethyl-[1,3]-dioxolane or (DLin-K-XTC2-DMA). In some aspects, the lipid formulation comprises an ionizable cationic lipid. In some aspects, the ionizable cationic lipid has a structure of Formula I:

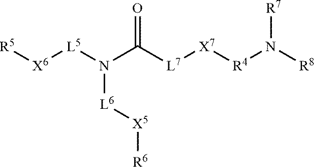

or a pharmaceutically acceptable salt or solvate thereof, wherein R⁵ and R⁶ are each independently selected from the group consisting of a linear or branched C₁₋C₃₁ alkyl, C₂₋C₃₁ alkenyl or C₂₋C₃₁ alkynyl and cholesteryl; L⁵ and L⁶ are each independently selected from the group consisting of a linear C₁₋C₂₀ alkyl and C₂₋C₂₀ alkenyl; X⁵ is —C(O)O—, whereby —C(O)OR⁶ is formed or —OC(O)— whereby —OC(O)—R⁶ is formed; X⁶ is —C(O)O— whereby —C(O)O—R⁵ is formed or —OC(O)— whereby —OC(O)—R⁵ is formed; X⁷ is S or O; L⁷ is absent or lower alkyl; R⁴ is a linear or branched C₁₋C₆ alkyl; and R⁷ and R⁸ are each independently selected from the group consisting of a hydrogen and a linear or branched C₁-C₆ alkyl. In some aspects, the ionizable cationic lipid is selected from

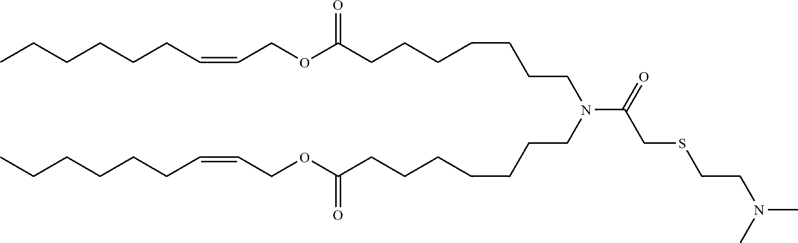 [SMILES: CCCCCC/C=C\COC(=O)CCCCCCCN(CCCCCCCC(=O)OC/C=C\CCCCCC)C(=O)CSCCN(C)C]

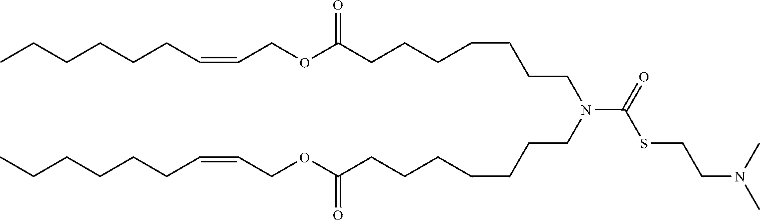 [SMILES: CCCCCC/C=C\COC(=O)CCCCCCCN(CCCCCCCC(=O)OC/C=C\CCCCCC)C(=O)SCCN(C)C]

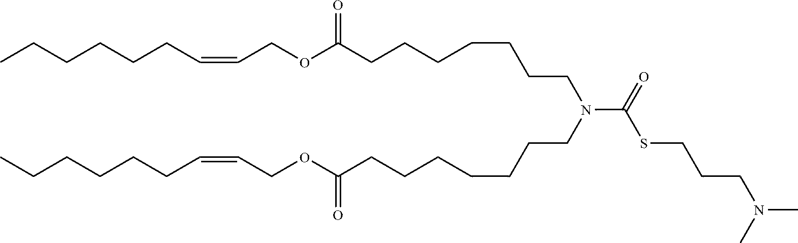 [SMILES: CCCCCC/C=C\COC(=O)CCCCCCCN(CCCCCCCC(=O)OC/C=C\CCCCCC)C(=O)SCCCN(C)C]

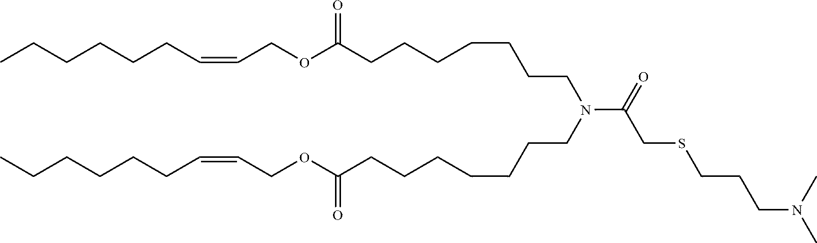 [SMILES: CCCCCC/C=C\COC(=O)CCCCCCCN(CCCCCCCC(=O)OC/C=C\CCCCCC)C(=O)CSCCCN(C)C]

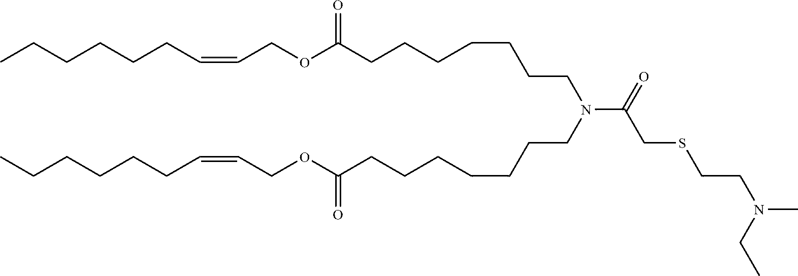 [SMILES: CCCCCC/C=C\COC(=O)CCCCCCCN(CCCCCCCC(=O)OC/C=C\CCCCCC)C(=O)CSCCN(C)CC]

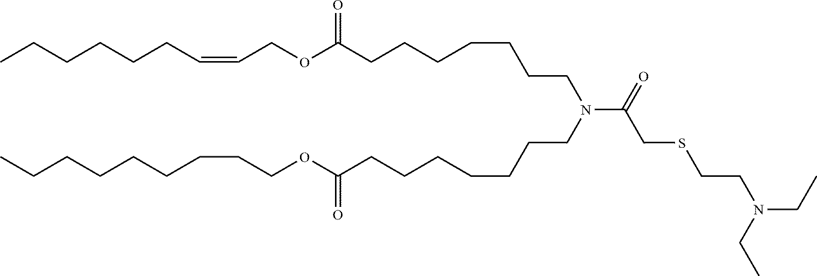 [SMILES: CCCCCC/C=C\COC(=O)CCCCCCCN(CCCCCCCC(=O)OCCCCCCCCC)C(=O)CSCCN(CC)CC]

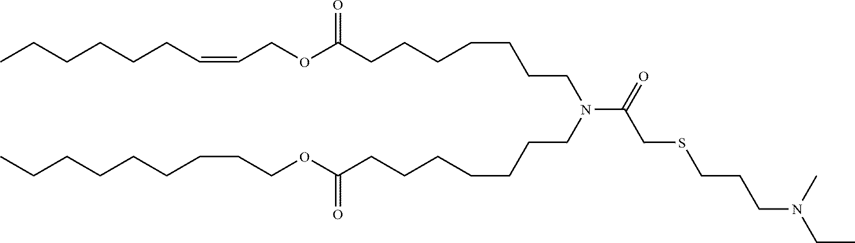 [SMILES: CCCCCC/C=C\COC(=O)CCCCCCCN(CCCCCCCC(=O)OCCCCCCCCC)C(=O)CSCCCN(C)CC]

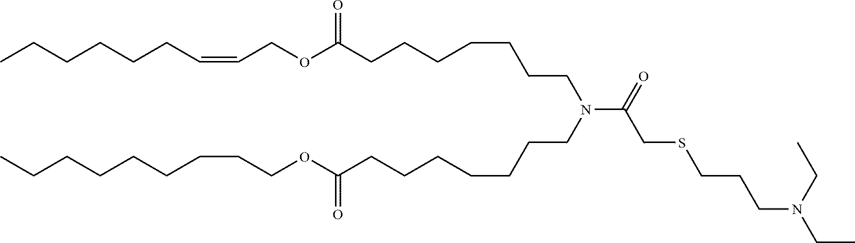 [SMILES: CCCCCC/C=C\COC(=O)CCCCCCCN(CCCCCCCC(=O)OCCCCCCCCC)C(=O)CSCCCN(CC)CC]

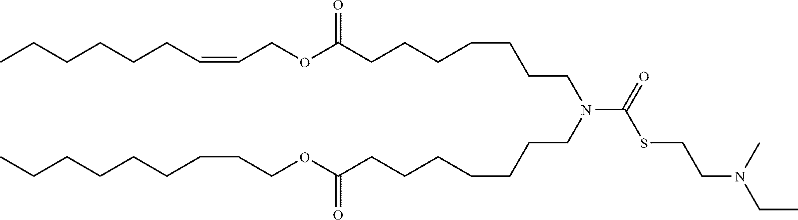 [SMILES: CCCCCC/C=C\COC(=O)CCCCCCCN(CCCCCCCC(=O)OCCCCCCCCC)C(=O)SCCN(C)CC]

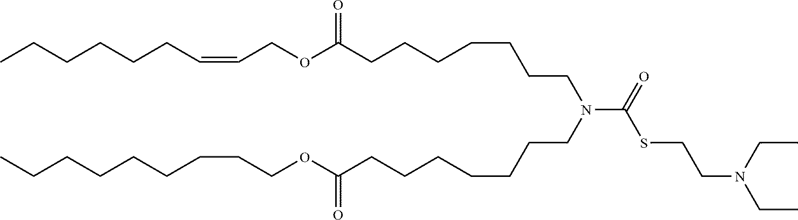 [SMILES: CCCCCC/C=C\COC(=O)CCCCCCCN(CCCCCCCC(=O)OCCCCCCCCC)C(=O)SCCN(CC)CC]

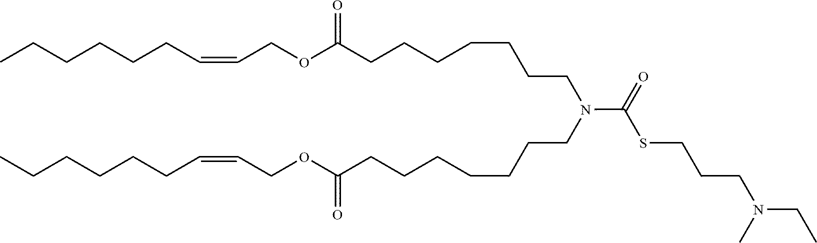 [SMILES: CCCCCC/C=C\COC(=O)CCCCCCCN(CCCCCCCC(=O)OC/C=C\CCCCCC)C(=O)SCCCN(C)CC]

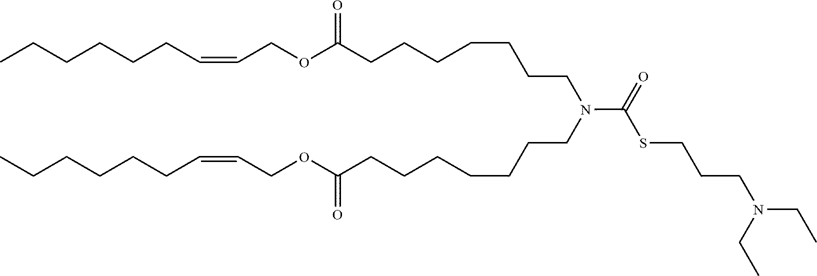 [SMILES: CCCCCC/C=C\COC(=O)CCCCCCCN(CCCCCCCC(=O)OC/C=C\CCCCCC)C(=O)SCCCN(CC)CC]

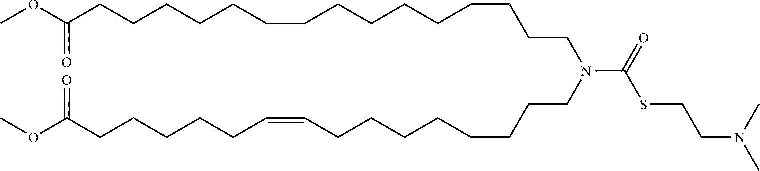

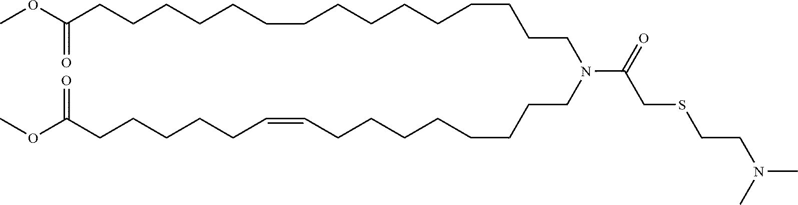

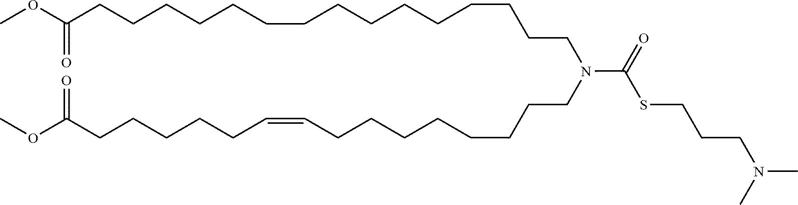

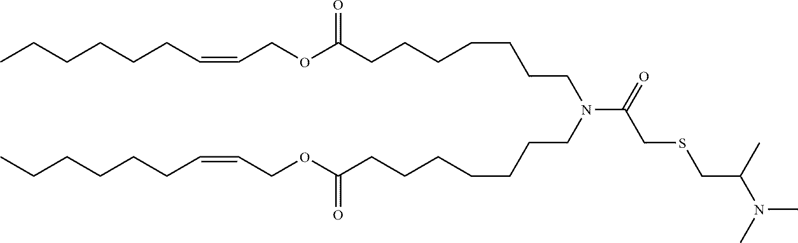 [SMILES: CCCCCC/C=C\COC(=O)CCCCCCCN(CCCCCCCC(=O)OC/C=C\CCCCCC)C(=O)CSCC(C)N(C)C]

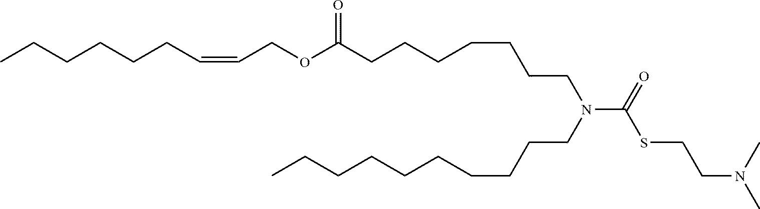 [SMILES: CCCCCC/C=C\COC(=O)CCCCCCCN(CCCCCCCCCC)C(=O)SCCN(C)C]

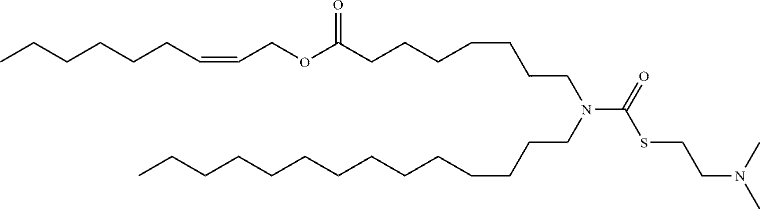 [SMILES: CCCCCC/C=C\COC(=O)CCCCCCCN(CCCCCCCCCCCCCC)C(=O)SCCN(C)C]

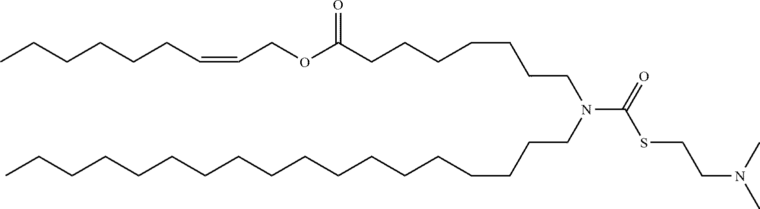 [SMILES: CCCCCC/C=C\COC(=O)CCCCCCCN(CCCCCCCCCCCCCCCCCC)C(=O)SCCN(C)C]

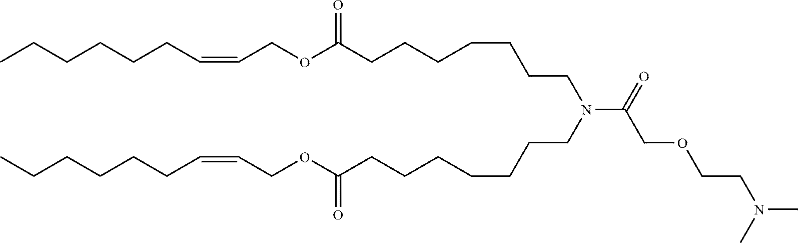 [SMILES: CCCCCC/C=C\COC(=O)CCCCCCCN(CCCCCCCC(=O)OC/C=C\CCCCCC)C(=O)COCCN(C)C]

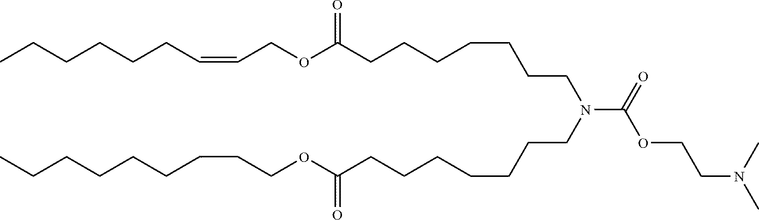 [SMILES: CCCCCC/C=C\COC(=O)CCCCCCCN(CCCCCCCC(=O)OCCCCCCCCC)C(=O)OCCN(C)C]

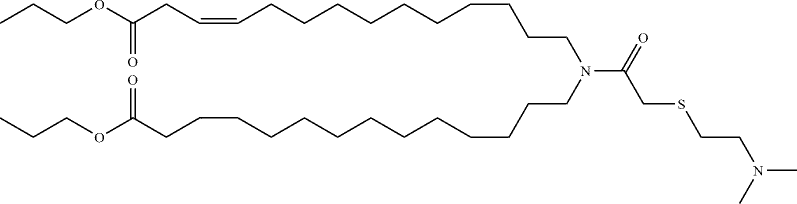 [SMILES: CCCOC(=O)C/C=C\CCCCCCCCCCN(CCCCCCCCCCCCCC(=O)OCCC)C(=O)CSCCN(C)C]

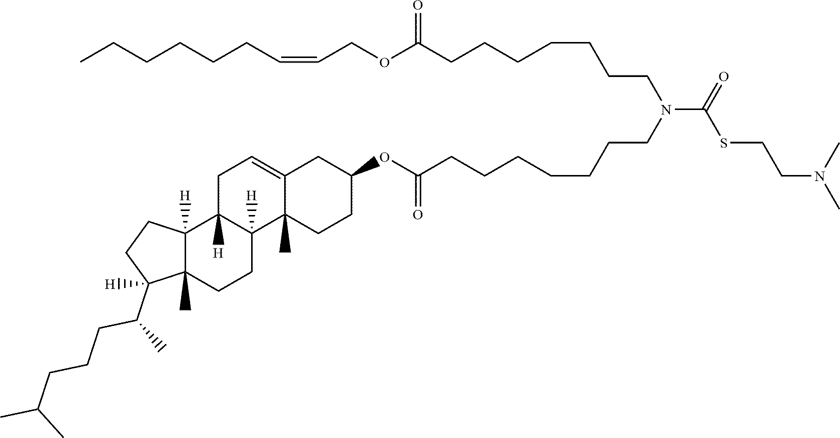

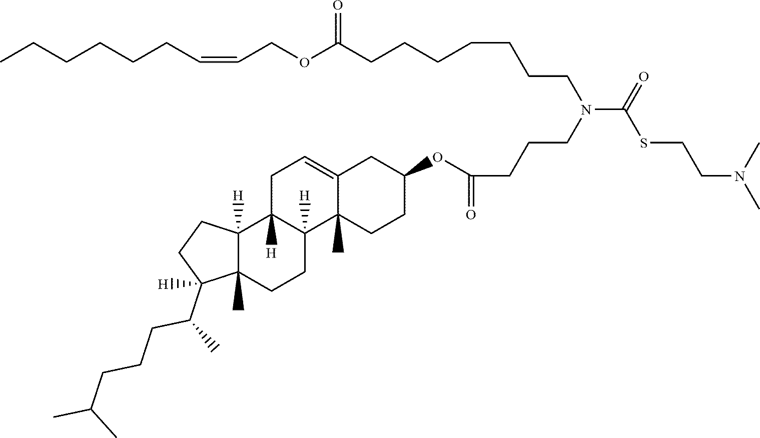

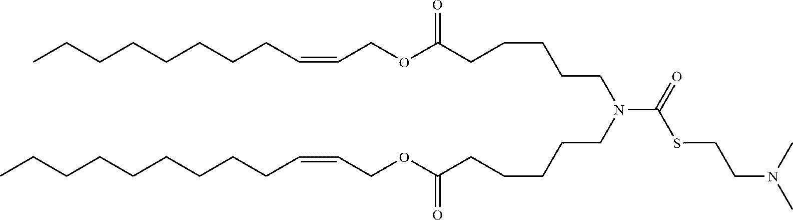 [SMILES: CCCCCCCC/C=C\COC(=O)CCCCCN(CCCCCC(=O)OC/C=C\CCCCCCCCC)C(=O)SCCN(C)C]

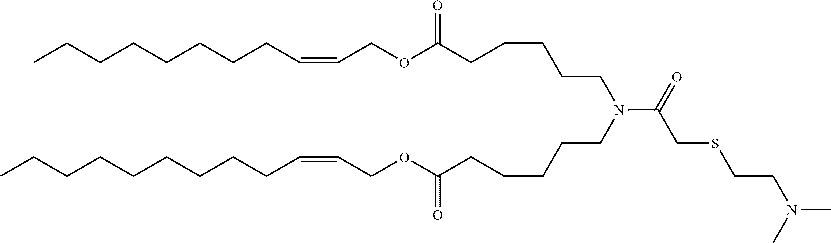 [SMILES: CCCCCCCC/C=C\COC(=O)CCCCCN(CCCCCC(=O)OC/C=C\CCCCCCCCC)C(=O)CSCCN(C)C]

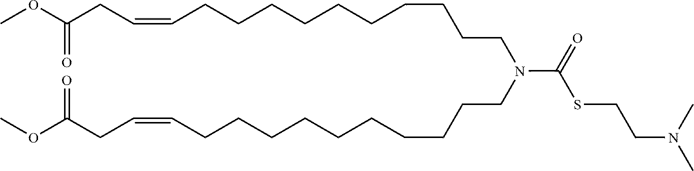

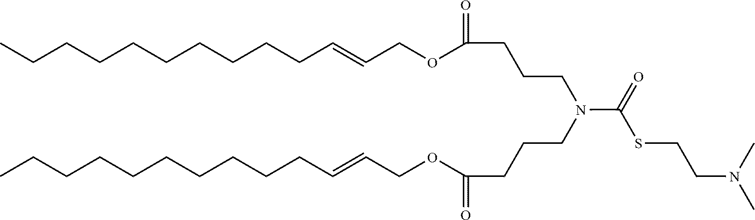 [SMILES: CCCCCCCCCC/C=C/COC(=O)CCCN(CCCC(=O)OC/C=C/CCCCCCCCCC)C(=O)SCCN(C)C]

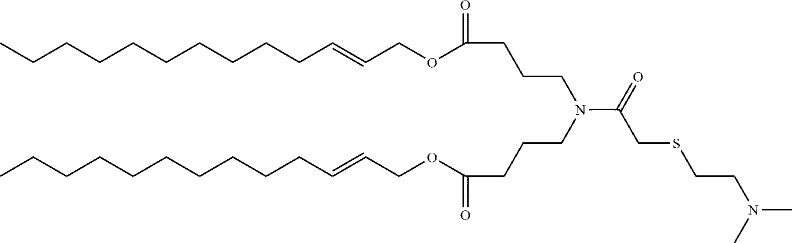 [SMILES: CCCCCCCCCC/C=C/COC(=O)CCCN(CCCC(=O)OC/C=C/CCCCCCCCCC)C(=O)CSCCN(C)C]

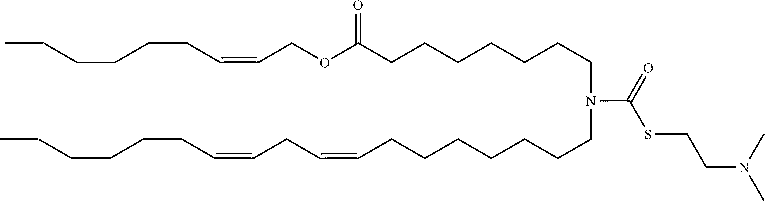 [SMILES: CCCCCC/C=C\C/C=C\CCCCCCCN(CCCCCCCC(=O)OC/C=C\CCCCCC)C(=O)SCCN(C)C]

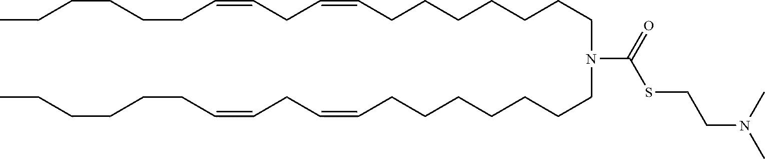 [SMILES: CCCCCC/C=C\C/C=C\CCCCCCCN(CCCCCCC/C=C\C/C=C\CCCCCC)C(=O)SCCN(C)C]

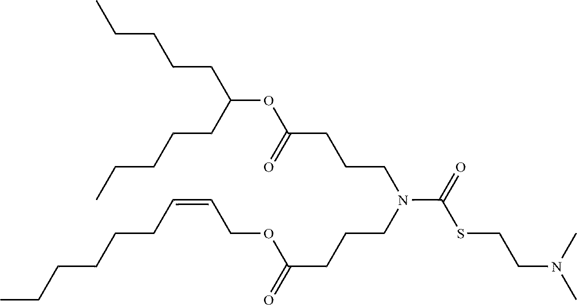 [SMILES: CCCCCC/C=C\COC(=O)CCCN(CCCC(=O)OC(CCCCC)CCCCC)C(=O)SCCN(C)C]

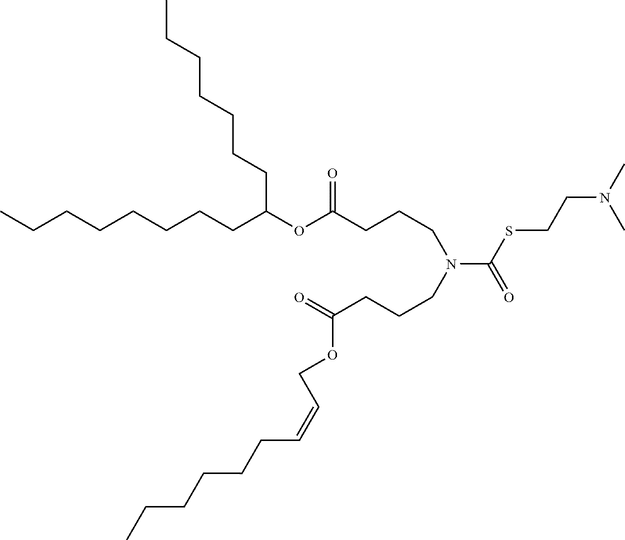 [SMILES: CCCCCC/C=C\COC(=O)CCCN(CCCC(=O)OC(CCCCCCC)CCCCCCCC)C(=O)SCCN(C)C]

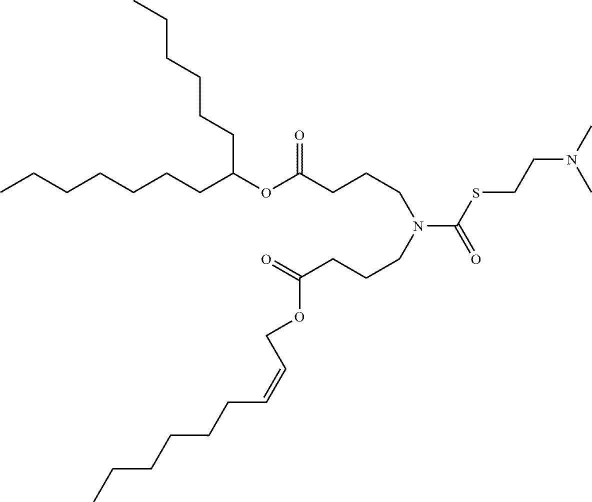 [SMILES: CCCCCC/C=C\COC(=O)CCCN(CCCC(=O)OC(CCCCCC)CCCCCCC)C(=O)SCCN(C)C]

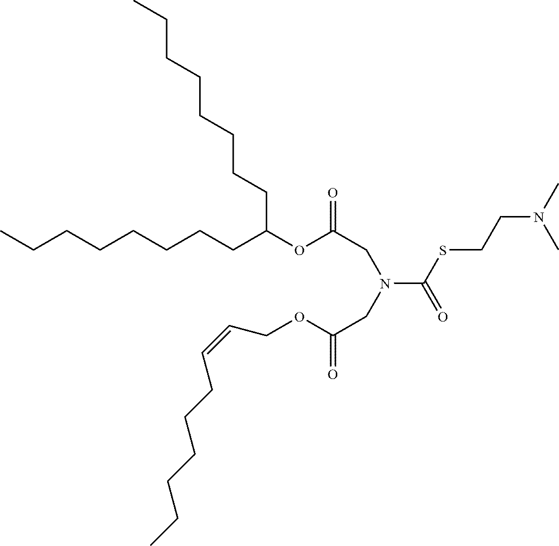 [SMILES: CCCCCC/C=C\COC(=O)CN(CC(=O)OC(CCCCCCCC)CCCCCCCC)C(=O)SCCN(C)C]

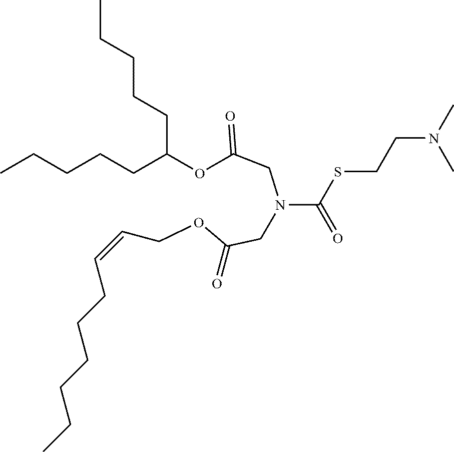 [SMILES: CCCCCC/C=C\COC(=O)CN(CC(=O)OC(CCCCC)CCCCC)C(=O)SCCN(C)C]

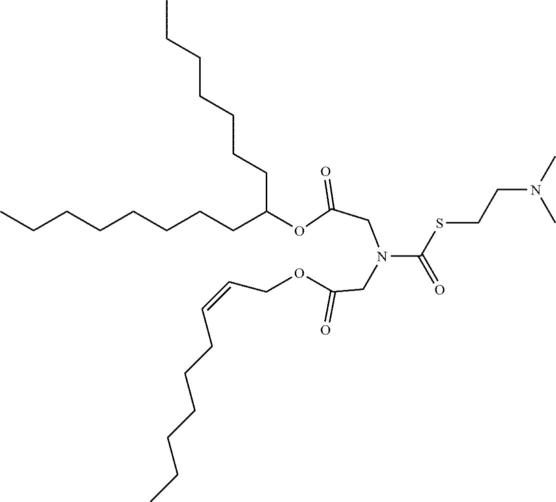 [SMILES: CCCCCC/C=C\COC(=O)CN(CC(=O)OC(CCCCCCC)CCCCCCCC)C(=O)SCCN(C)C]

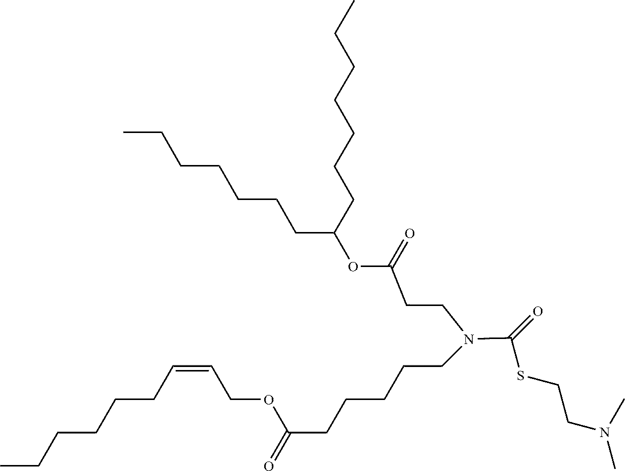 [SMILES: CCCCCC/C=C\COC(=O)CCCCCN(CCC(=O)OC(CCCCCCC)CCCCCCC)C(=O)SCCN(C)C]

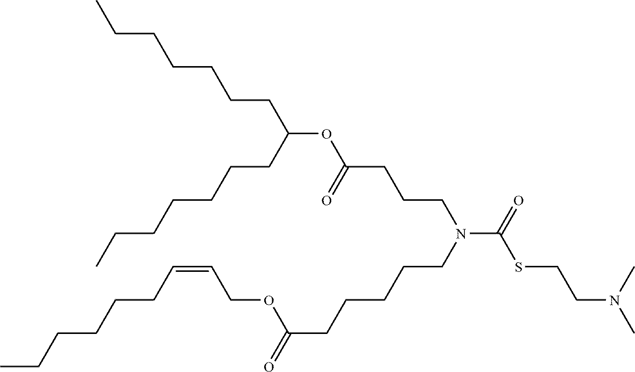 [SMILES: CCCCCC/C=C\COC(=O)CCCCCN(CCCC(=O)OC(CCCCCCC)CCCCCCC)C(=O)SCCN(C)C]

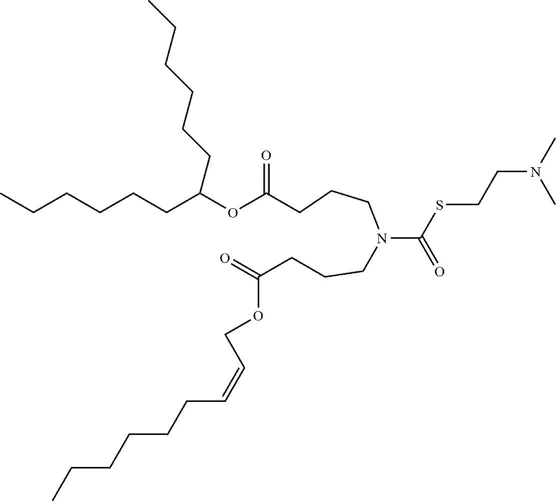 [SMILES: CCCCCC/C=C\COC(=O)CCCN(CCCC(=O)OC(CCCCCC)CCCCCC)C(=O)SCCN(C)C]

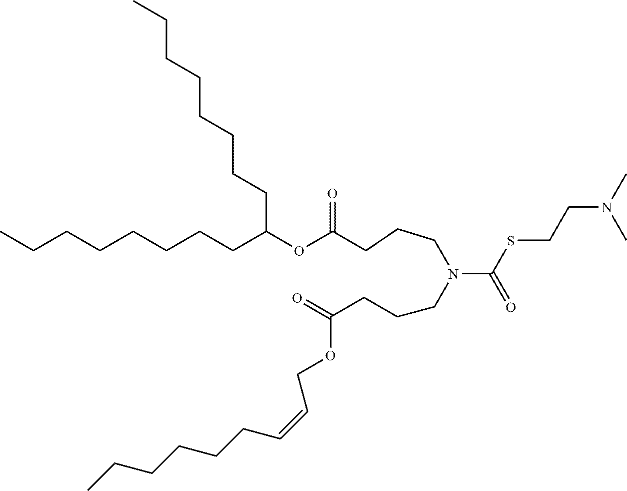

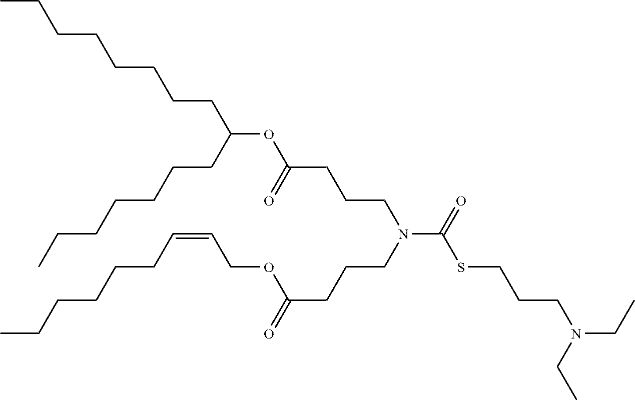 [SMILES: CCCCCC/C=C\COC(=O)CCCN(CCCC(=O)OC(CCCCCCC)CCCCCCCC)C(=O)SCCCN(CC)CC]

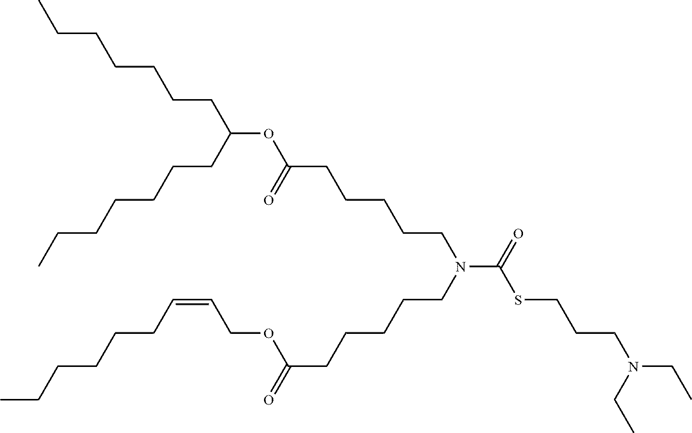 [SMILES: CCCCCC/C=C\COC(=O)CCCCCN(CCCCCC(=O)OC(CCCCCCC)CCCCCCC)C(=O)SCCCN(CC)CC]

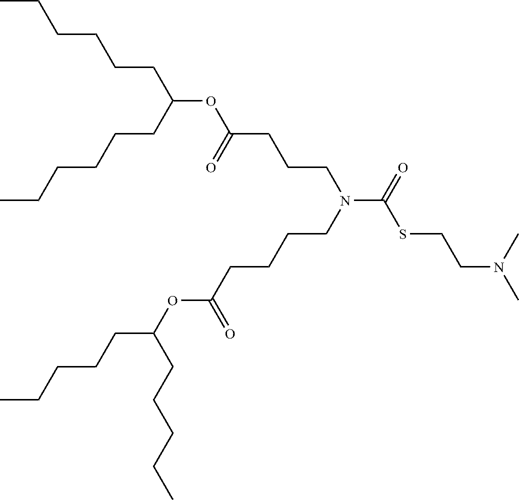 [SMILES: CCCCCCC(CCCCCC)OC(=O)CCCN(CCCCC(=O)OC(CCCCC)CCCCC)C(=O)SCCN(C)C]

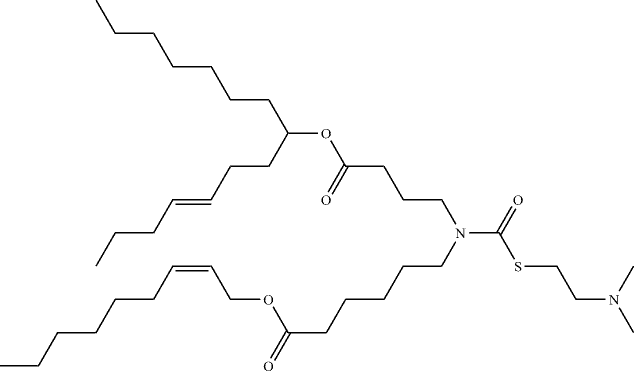 [SMILES: CCC/C=C/CCC(CCCCCCC)OC(=O)CCCN(CCCCCC(=O)OC/C=C\CCCCCC)C(=O)SCCN(C)C]

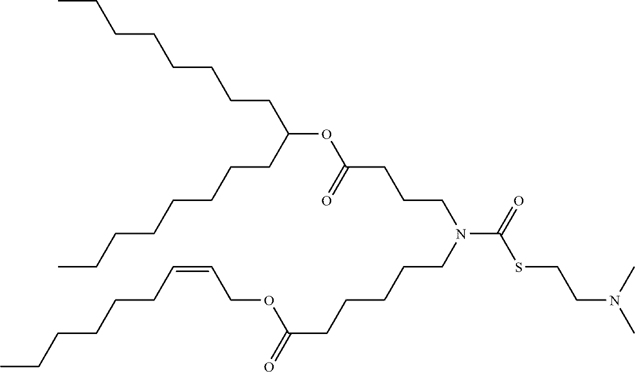 [SMILES: CCCCCC/C=C\COC(=O)CCCCCN(CCCC(=O)OC(CCCCCCCC)CCCCCCCC)C(=O)SCCN(C)C]

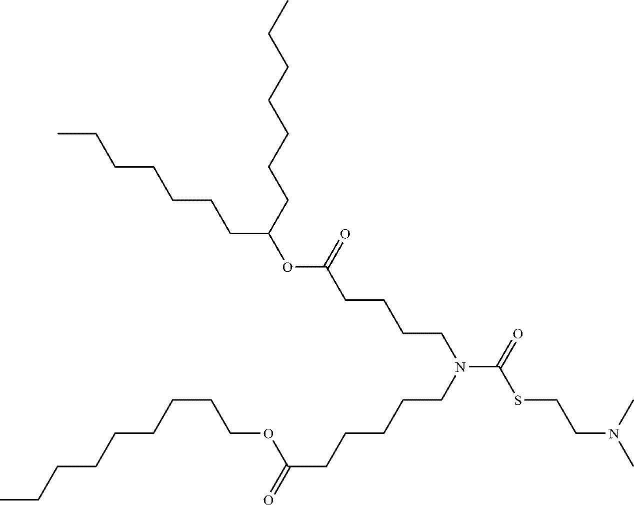 [SMILES: CCCCCCCCCOC(=O)CCCCCN(CCCCC(=O)OC(CCCCCCC)CCCCCCC)C(=O)SCCN(C)C]

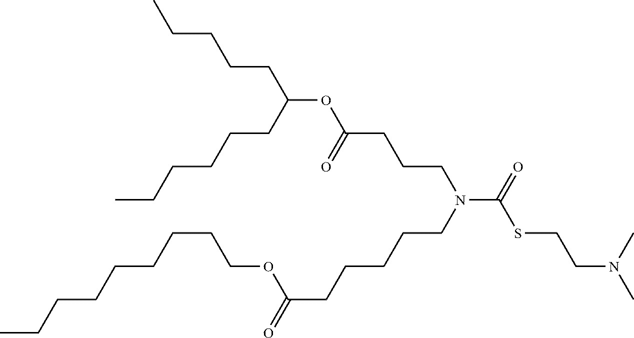 [SMILES: CCCCCCCCCOC(=O)CCCCCN(CCCC(=O)OC(CCCCC)CCCCCC)C(=O)SCCN(C)C]

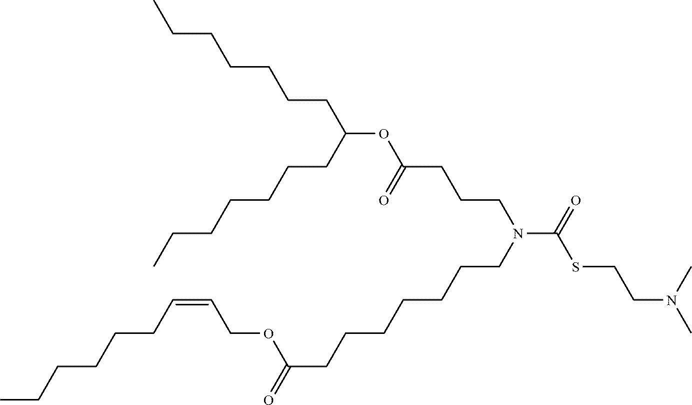

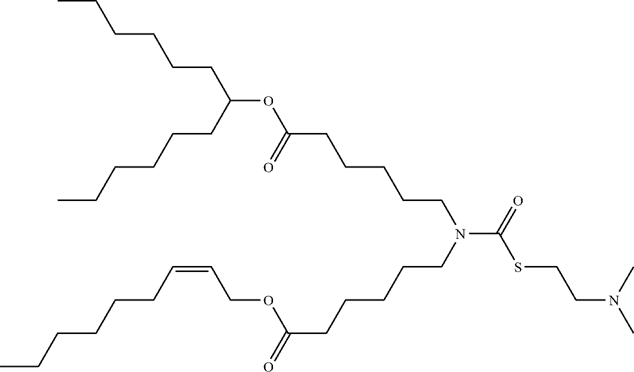 [SMILES: CCCCCC/C=C\COC(=O)CCCCCN(CCCCCC(=O)OC(CCCCCC)CCCCCC)C(=O)SCCN(C)C]

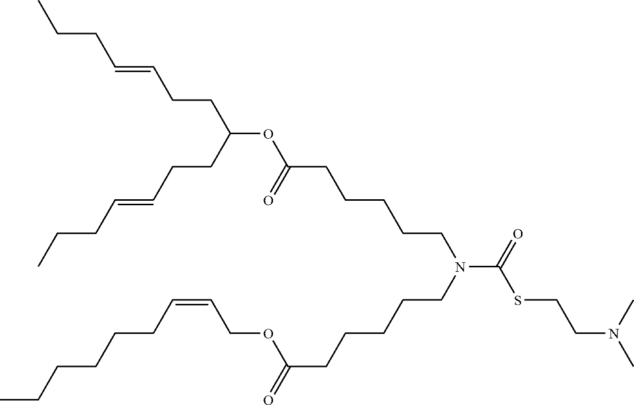 [SMILES: CCC/C=C/CCC(CC/C=C/CCC)OC(=O)CCCCCN(CCCCCC(=O)OC/C=C\CCCCCC)C(=O)SCCN(C)C]

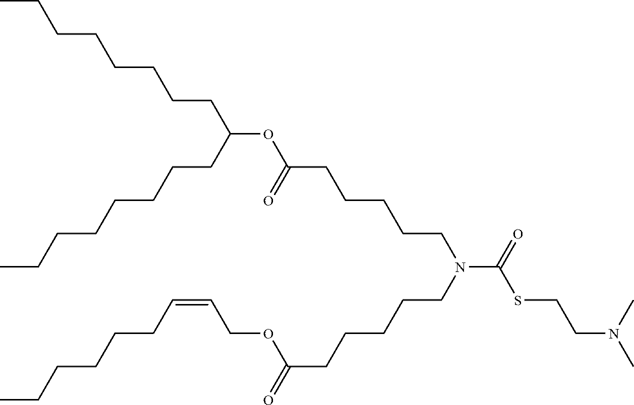 [SMILES: CCCCCC/C=C\COC(=O)CCCCCN(CCCCCC(=O)OC(CCCCCCCC)CCCCCCCC)C(=O)SCCN(C)C]

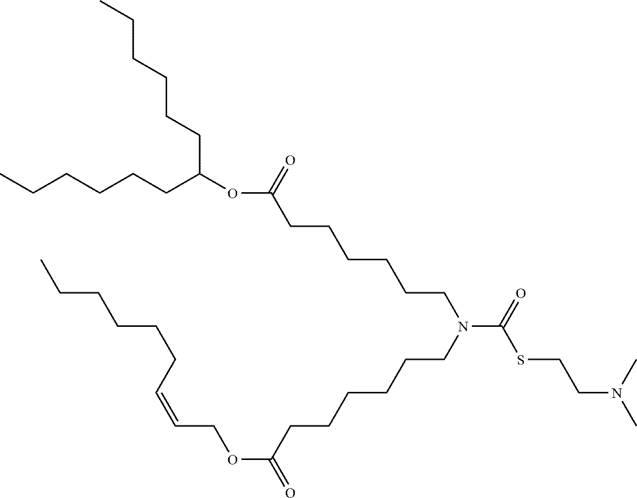 [SMILES: CCCCCC/C=C\COC(=O)CCCCCCN(CCCCCCC(=O)OC(CCCCCC)CCCCCC)C(=O)SCCN(C)C]

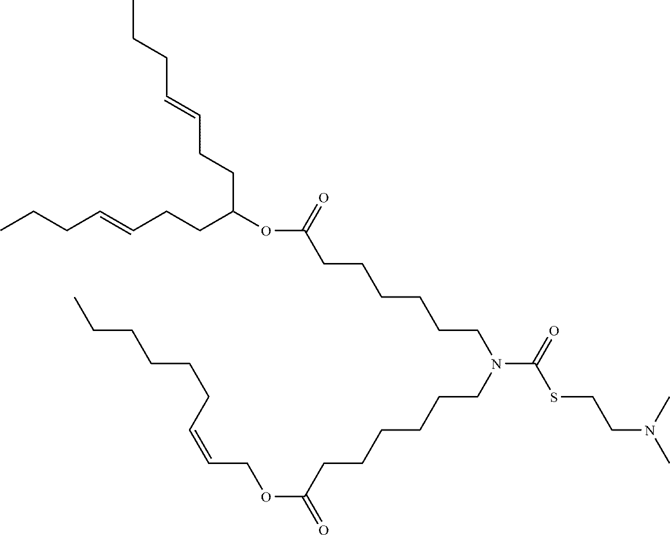 [SMILES: CCC/C=C/CCC(CC/C=C/CCC)OC(=O)CCCCCCN(CCCCCCC(=O)OC/C=C\CCCCCC)C(=O)SCCN(C)C]

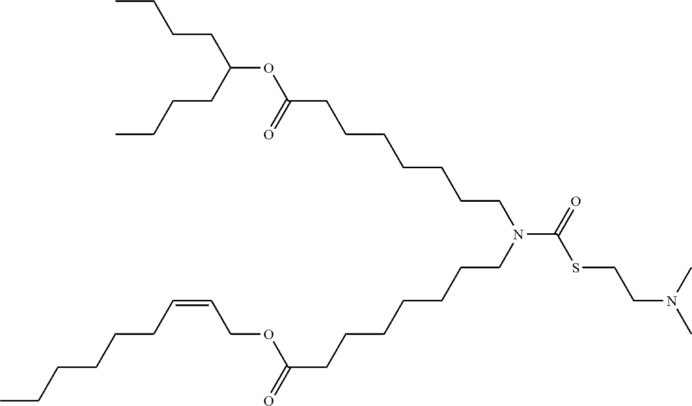 [SMILES: CCCCCC/C=C\COC(=O)CCCCCCCN(CCCCCCCC(=O)OC(CCCC)CCCC)C(=O)SCCN(C)C]

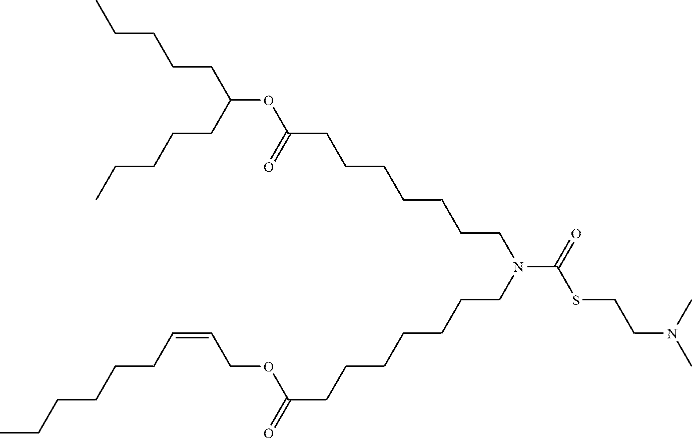 [SMILES: CCCCCC/C=C\COC(=O)CCCCCCCN(CCCCCCCC(=O)OC(CCCCC)CCCCC)C(=O)SCCN(C)C]

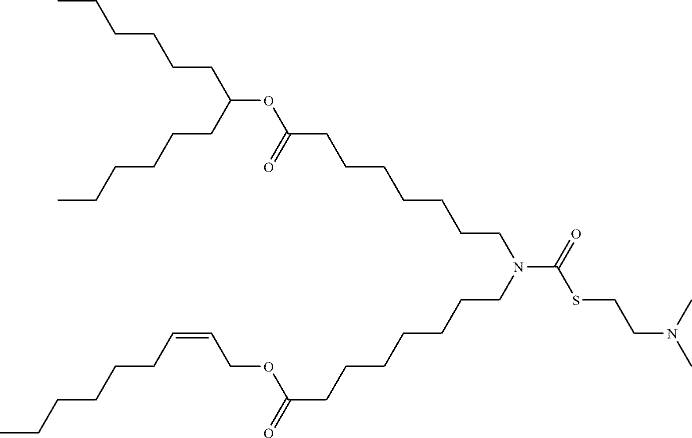 [SMILES: CCCCCC/C=C\COC(=O)CCCCCCCN(CCCCCCCC(=O)OC(CCCCCC)CCCCCC)C(=O)SCCN(C)C]

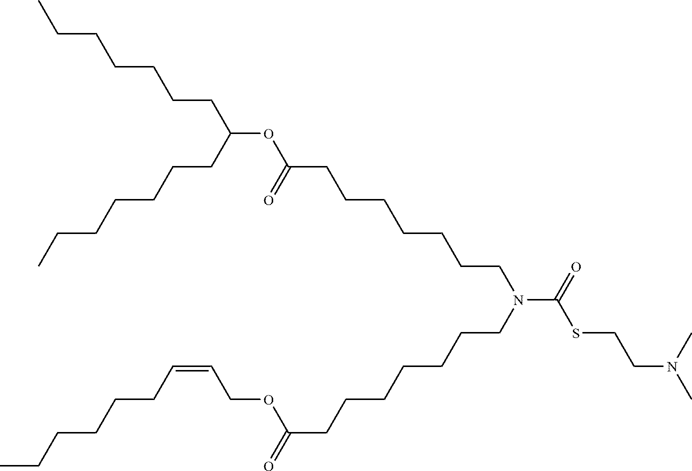 [SMILES: CCCCCC/C=C\COC(=O)CCCCCCCN(CCCCCCCC(=O)OC(CCCCCCC)CCCCCCC)C(=O)SCCN(C)C]

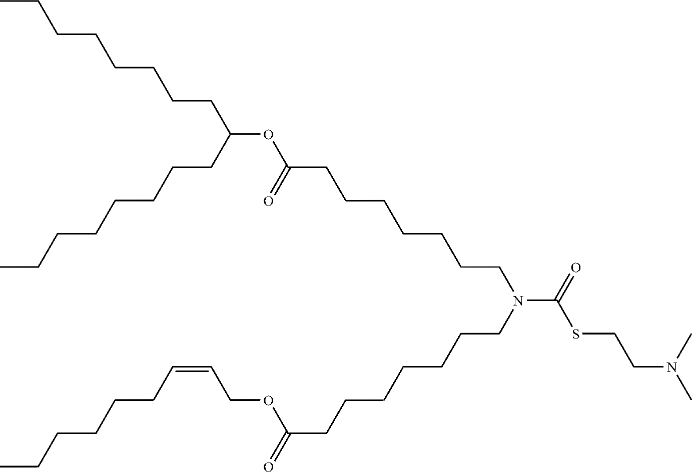 [SMILES: CCCCCC/C=C\COC(=O)CCCCCCCN(CCCCCCCC(=O)OC(CCCCCCCC)CCCCCCCC)C(=O)SCCN(C)C]

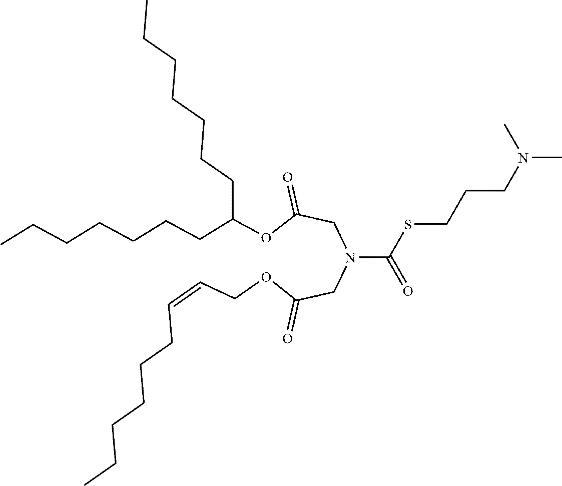 [SMILES: CCCCCC/C=C\COC(=O)CN(CC(=O)OC(CCCCCCC)CCCCCCC)C(=O)SCCCN(C)C]

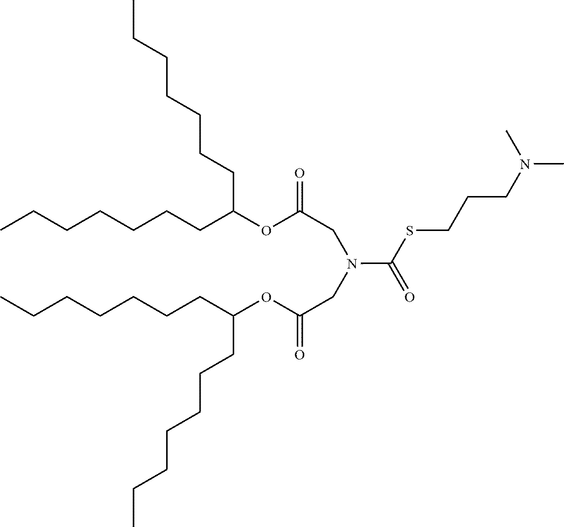 [SMILES: CCCCCCCC(CCCCCCC)OC(=O)CN(CC(=O)OC(CCCCCCC)CCCCCCC)C(=O)SCCCN(C)C]

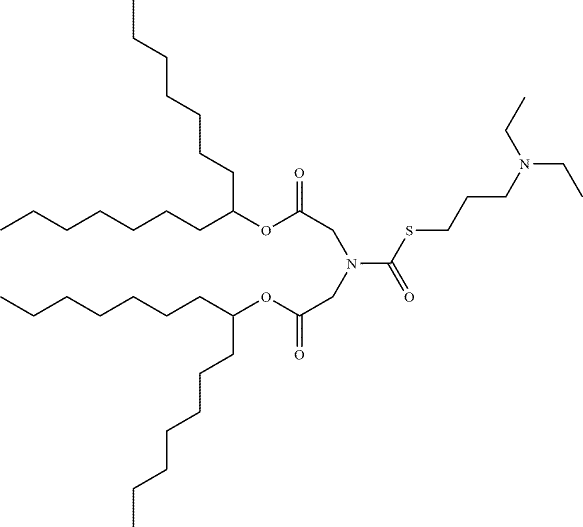 [SMILES: CCCCCCCC(CCCCCCC)OC(=O)CN(CC(=O)OC(CCCCCCC)CCCCCCC)C(=O)SCCCN(CC)CC]

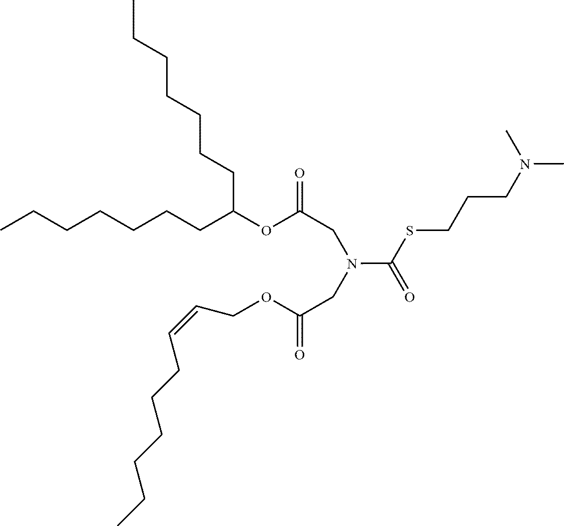 [SMILES: CCCCCC/C=C\COC(=O)CN(CC(=O)OC(CCCCCCC)CCCCCCC)C(=O)SCCCN(C)C]

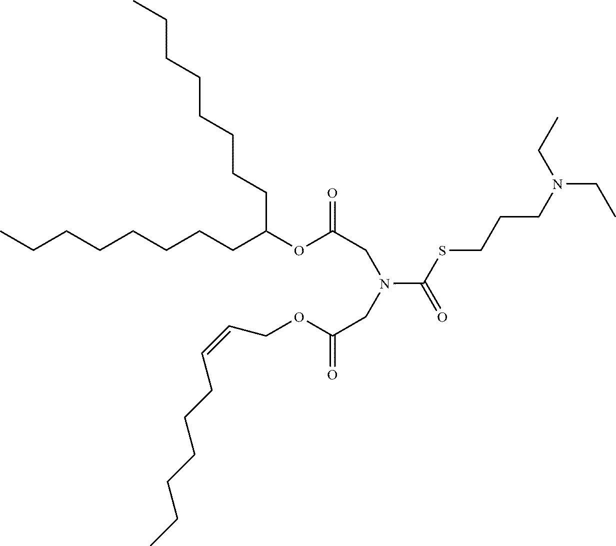 [SMILES: CCCCCC/C=C\COC(=O)CN(CC(=O)OC(CCCCCCCC)CCCCCCCC)C(=O)SCCCN(CC)CC]

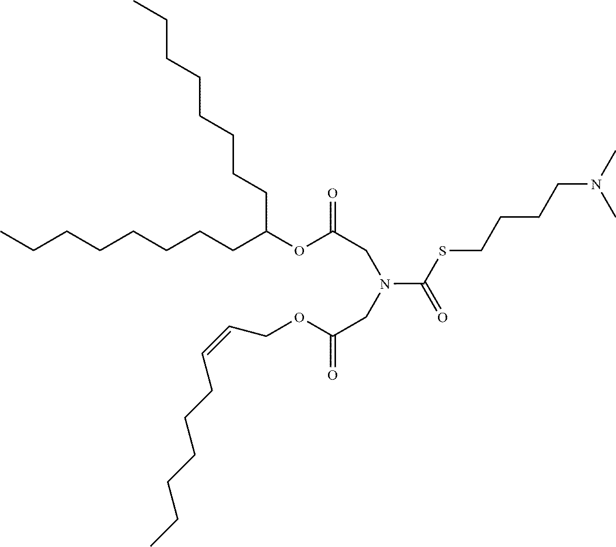 [SMILES: CCCCCC/C=C\COC(=O)CN(CC(=O)OC(CCCCCCCC)CCCCCCCC)C(=O)SCCCCN(C)C]

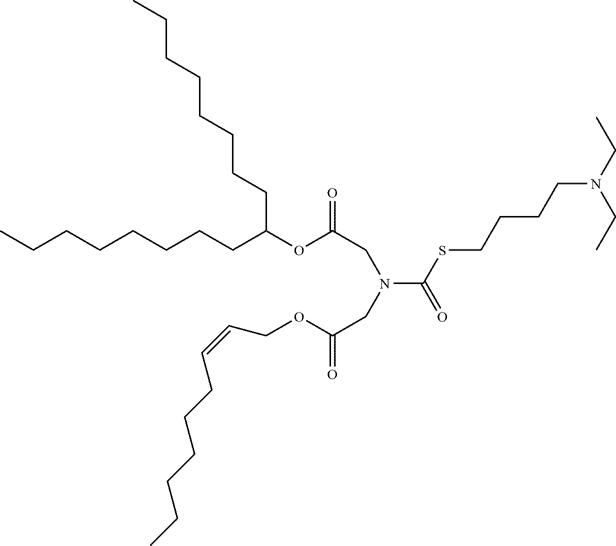 [SMILES: CCCCCC/C=C\COC(=O)CN(CC(=O)OC(CCCCCCCC)CCCCCCCC)C(=O)SCCCCN(CC)CC]

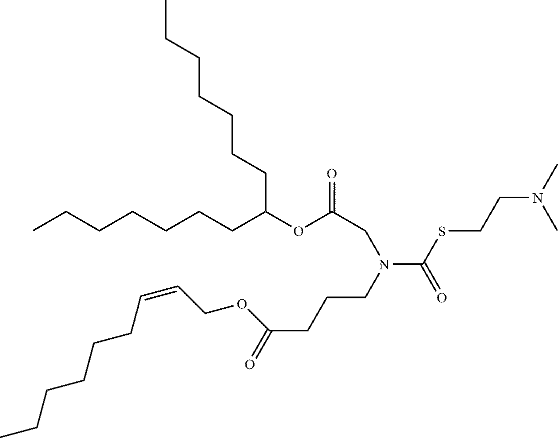 [SMILES: CCCCCC/C=C\COC(=O)CCCN(CC(=O)OC(CCCCCCC)CCCCCCC)C(=O)SCCN(C)C]

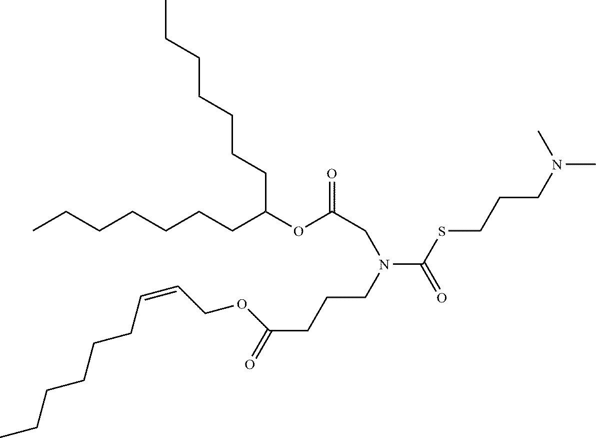 [SMILES: CCCCCC/C=C\COC(=O)CCCN(CC(=O)OC(CCCCCCC)CCCCCCC)C(=O)SCCCN(C)C]

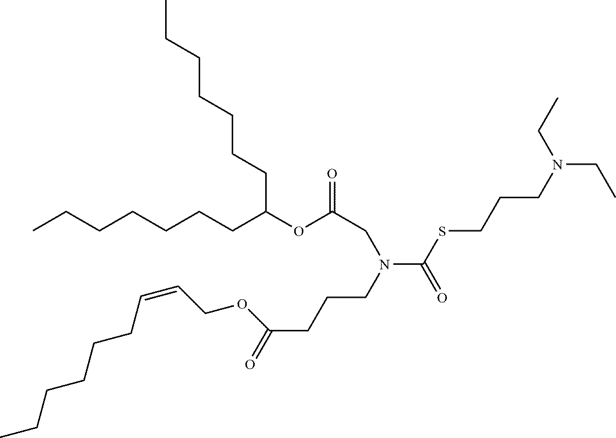 [SMILES: CCCCCC/C=C\COC(=O)CCCN(CC(=O)OC(CCCCCCC)CCCCCCC)C(=O)SCCCN(CC)CC]

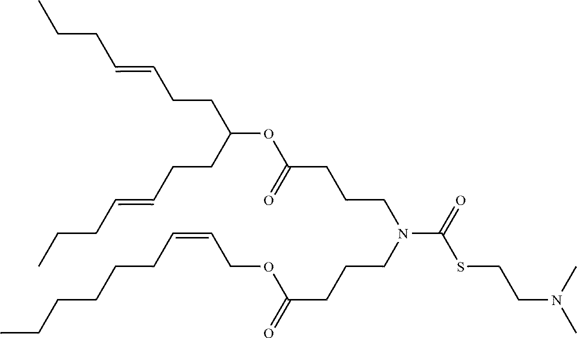 [SMILES: CCC/C=C/CCC(CC/C=C/CCC)OC(=O)CCCN(CCCC(=O)OC/C=C\CCCCCC)C(=O)SCCN(C)C]

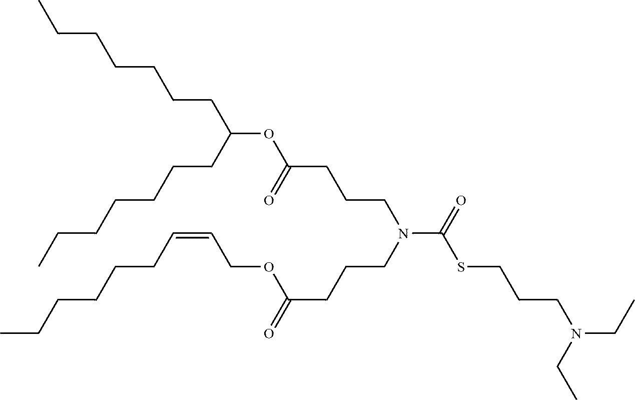 [SMILES: CCCCCC/C=C\COC(=O)CCCN(CCCC(=O)OC(CCCCCCC)CCCCCCC)C(=O)SCCCN(CC)CC]

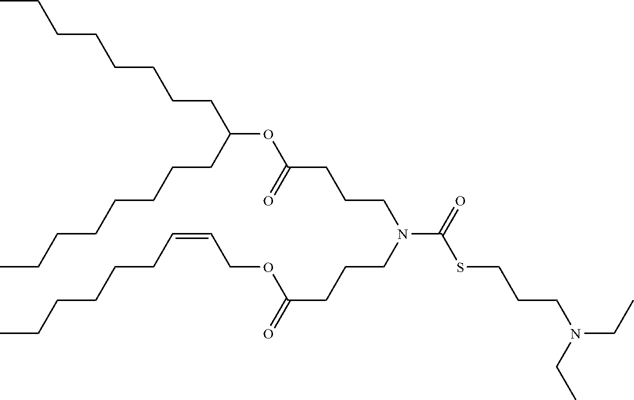 [SMILES: CCCCCC/C=C\COC(=O)CCCN(CCCC(=O)OC(CCCCCCCC)CCCCCCCC)C(=O)SCCCN(CC)CC]

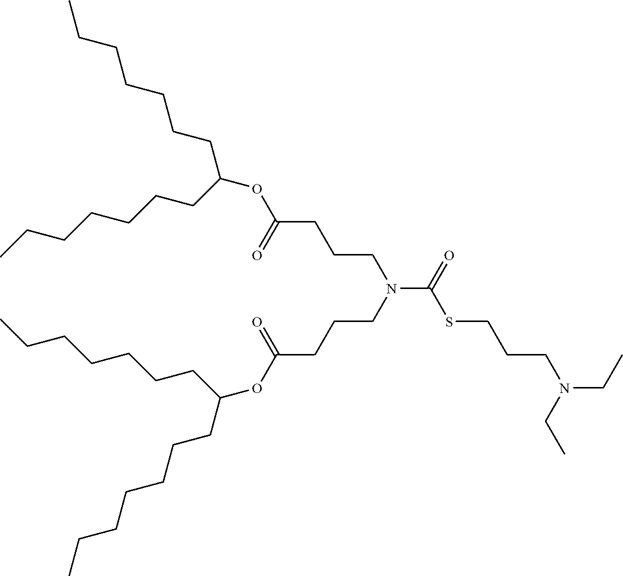 [SMILES: CCCCCCCC(CCCCCCC)OC(=O)CCCN(CCCC(=O)OC(CCCCCCC)CCCCCCC)C(=O)SCCCN(CC)CC]

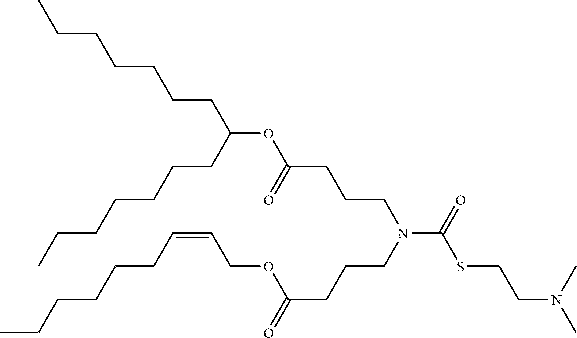 [SMILES: CCCCCC/C=C\COC(=O)CCCN(CCCC(=O)OC(CCCCCCC)CCCCCCC)C(=O)SCCN(C)C]

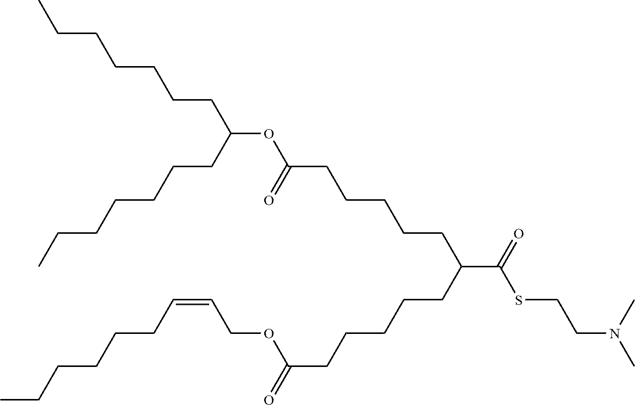 [SMILES: CCCCCC/C=C\COC(=O)CCCCCC(CCCCCC(=O)OC(CCCCCCC)CCCCCCC)C(=O)SCCN(C)C]

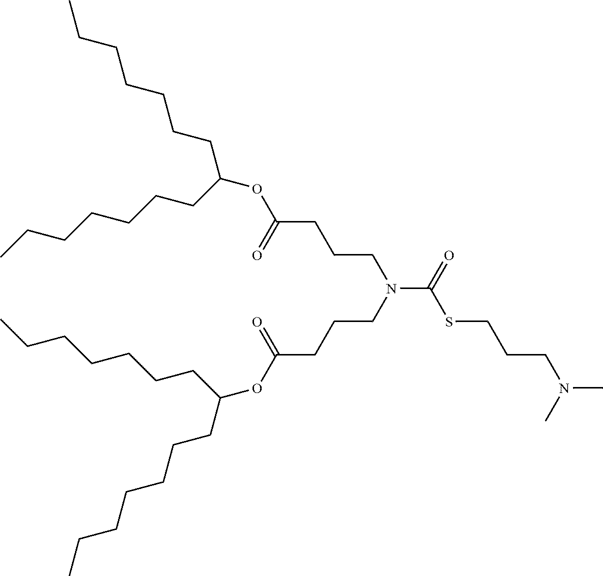 [SMILES: CCCCCCCC(CCCCCCC)OC(=O)CCCN(CCCC(=O)OC(CCCCCCC)CCCCCCC)C(=O)SCCCN(C)C]

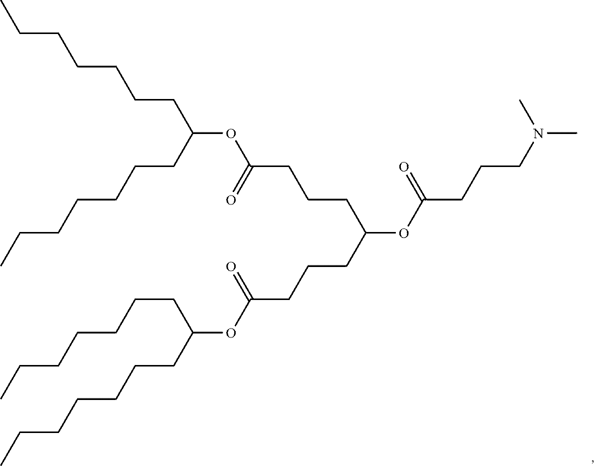

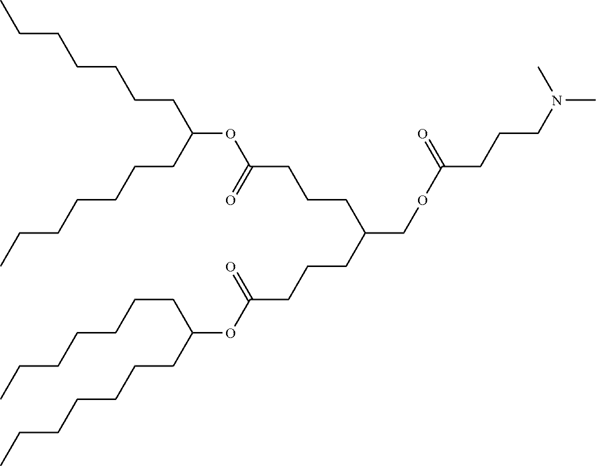 [SMILES: CCCCCCCC(CCCCCCC)OC(=O)CCCC(CCCC(=O)OC(CCCCCCC)CCCCCCC)COC(=O)CCCN(C)C]

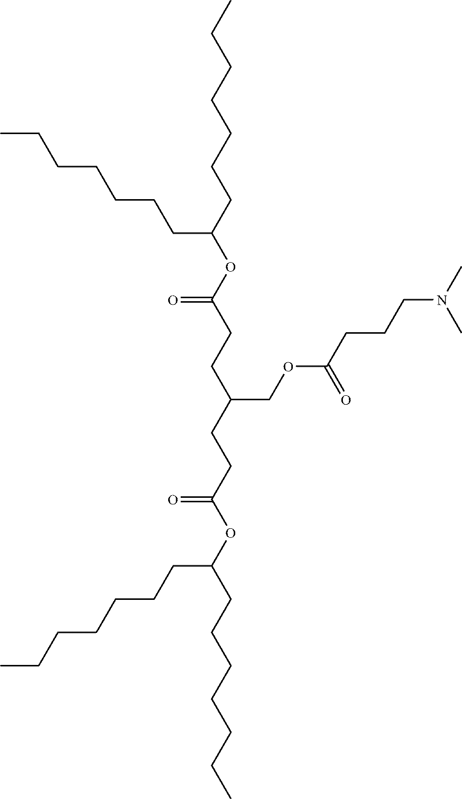 [SMILES: CCCCCCCC(CCCCCCC)OC(=O)CCC(CCC(=O)OC(CCCCCCC)CCCCCCC)COC(=O)CCCN(C)C]

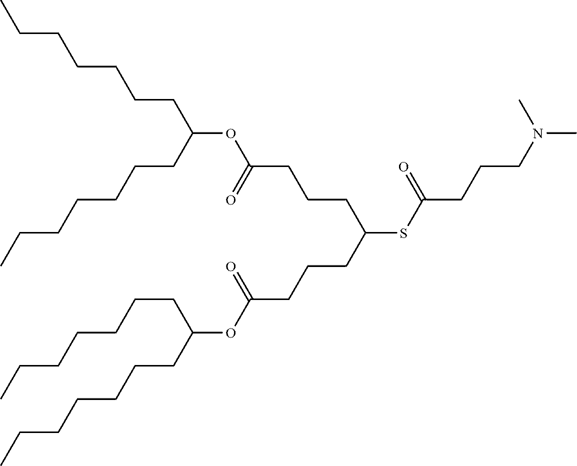 [SMILES: CCCCCCCC(CCCCCCC)OC(=O)CCCC(CCCC(=O)OC(CCCCCCC)CCCCCCC)SC(=O)CCCN(C)C]

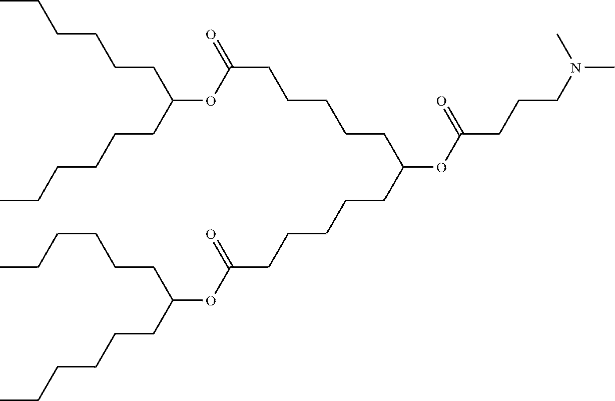 [SMILES: CCCCCCC(CCCCCC)OC(=O)CCCCCC(CCCCCC(=O)OC(CCCCCC)CCCCCC)OC(=O)CCCN(C)C]

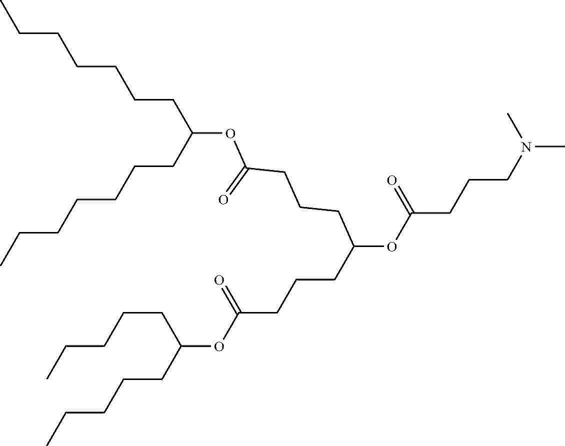 [SMILES: CCCCCCCC(CCCCCCC)OC(=O)CCCC(CCCC(=O)OC(CCCCC)CCCCC)OC(=O)CCCN(C)C]

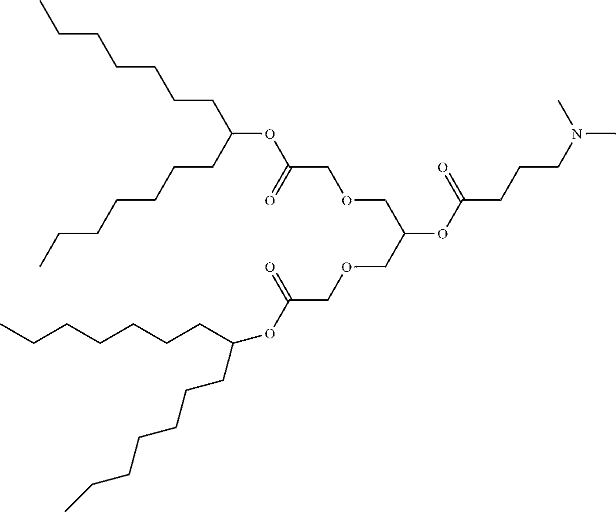 [SMILES: CCCCCCCC(CCCCCCC)OC(=O)COCC(COCC(=O)OC(CCCCCCC)CCCCCCC)OC(=O)CCCN(C)C]

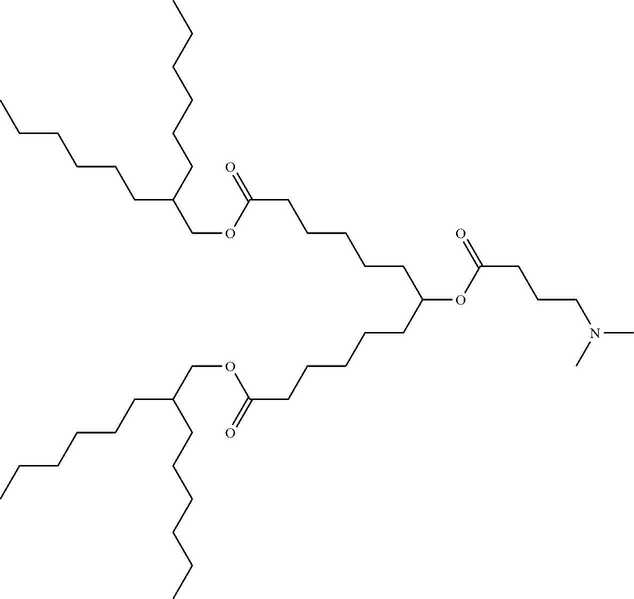 [SMILES: CCCCCCC(CCCCCC)COC(=O)CCCCCC(CCCCCC(=O)OCC(CCCCCC)CCCCCC)OC(=O)CCCN(C)C]

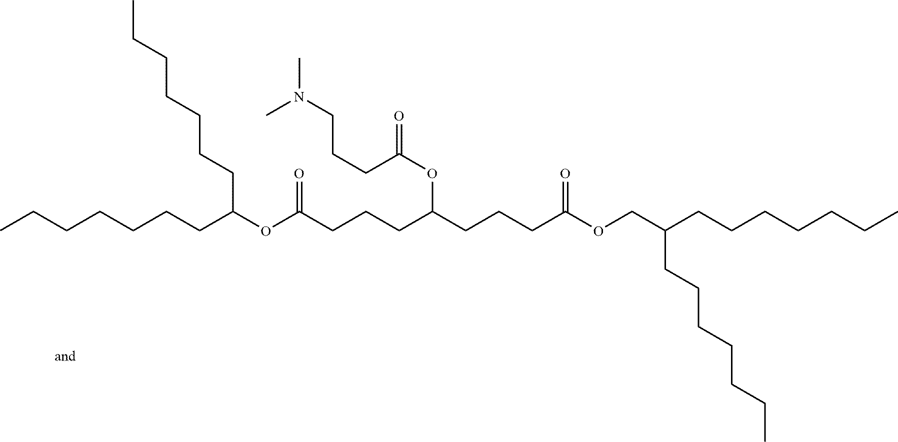

In some aspects, the ionizable cationic lipid is ATX-126:

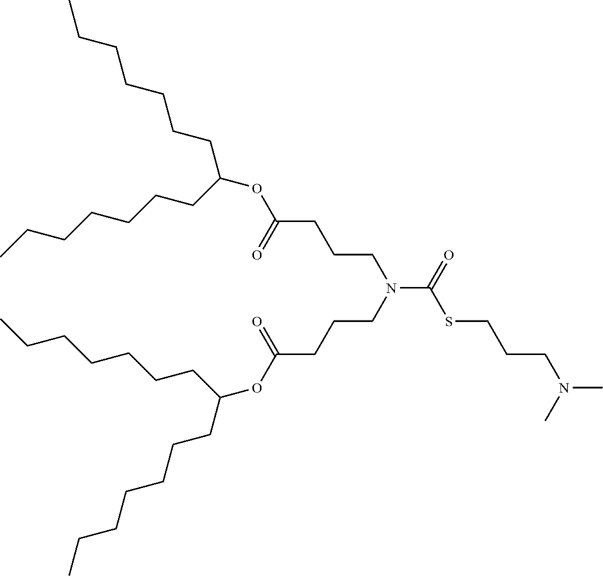 [SMILES: CCCCCCCC(CCCCCCC)OC(=O)CCCN(CCCC(=O)OC(CCCCCCC)CCCCCCC)C(=O)SCCCN(C)C]

In some aspects, the lipid formulation of compositions provided herein encapsulates the nucleic acid molecule. In some aspects, the lipid formulation is complexed to the nucleic acid molecule.

In some aspects, the lipid formulation further comprises a helper lipid. In some aspects, the helper lipid is a phospholipid. In some aspects, the helper lipid is selected from dioleoylphosphatidyl ethanolamine (DOPE), dimyristoylphosphatidyl choline (DMPC), distearoylphosphatidyl choline (DSPC), dimyristoylphosphatidyl glycerol (DMPG), dipalmitoyl phosphatidylcholine (DPPC), and phosphatidylcholine (PC). In some aspects, the helper lipid is distearoylphosphatidylcholine (DSPC).

In some aspects, the lipid formulation of compositions provided herein further comprises cholesterol. In some aspects, the lipid formulation further comprises a polyethylene glycol (PEG)-lipid conjugate. In some aspects, the PEG-lipid conjugate is PEG-DMG. In some aspects, the PEG-DMG is PEG2000-DMG.

In some aspects, the lipid portion of the lipid formulation comprises about 40 mol% to about 60 mol% of the ionizable cationic lipid, about 4 mol% to about 16 mol% DSPC, about 30 mol% to about 47 mol% cholesterol, and about 0.5 mol% to about 3 mol% PEG2000-DMG. In some aspects, the lipid portion of the lipid formulation comprises about 42 mol% to about 58 mol% of the ionizable cationic lipid, about 6 mol% to about 14 mol% DSPC, about 32 mol% to about 44 mol% cholesterol, and about 1 mol% to about 2 mol% PEG2000-DMG. In some aspects, the lipid portion of the lipid formulation comprises about 45 mol% to about 55 mol% of the ionizable cationic lipid, about 8 mol% to about 12 mol% DSPC, about 35 mol% to about 42 mol% cholesterol, and about 1.25 mol% to about 1.75 mol% PEG2000-DMG.

In some aspects, the composition has a total lipid:nucleic acid molecule weight ratio of about 50:1 to about 10:1. In some aspects, the composition has a total lipid:nucleic acid molecule weight ratio of about 44:1 to about 24:1. In some aspects, the composition has a total lipid: nucleic acid molecule weight ratio of about 40:1 to about 28:1. In some aspects, the composition has a total lipid: nucleic acid molecule weight ratio of about 38:1 to about 30:1. In some aspects, the composition has a total lipid: nucleic acid molecule weight ratio of about 37:1 to about 33:1.

In some aspects, the composition comprises a HEPES or TRIS buffer at a pH of about 7.0 to about 8.5. In some aspects, the HEPES or TRIS buffer is at a concentration of about 7 mg/mL to about 15 mg/mL.

In some aspects, the composition further comprises about 2.0 mg/mL to about 4.0 mg/mL of NaCl. In some aspects, the composition further comprises one or more cryoprotectants. In some aspects, the one or more cryoprotectants are selected from sucrose, glycerol, or a combination of sucrose and glycerol. In some aspects, the composition comprises a combination of sucrose at a concentration of about 70 mg/mL to about 110 mg/mL and glycerol at a concentration of about 50 mg/mL to about 70 mg/mL.

In some aspects, the composition is a lyophilized composition. In some aspects, the lyophilized composition comprises one or more lyoprotectants. In some aspects, the lyophilized composition comprises a poloxamer, potassium sorbate, sucrose, or any combination thereof. In some aspects, the poloxamer is poloxamer 188.

In some aspects, the lyophilized composition comprises about 0.01 to about 1.0 % w/w of the RNA molecule. In some aspects, the lyophilized composition comprises about 1.0 to about 5.0 % w/w lipids. In some aspects, the lyophilized composition comprises about 0.5 to about 2.5 % w/w of TRIS buffer. In some aspects, the lyophilized composition comprises about 0.75 to about 2.75 % w/w of NaCl. In some aspects, the lyophilized composition comprises about 85 to about 95 % w/w of a sugar. In some aspects, the sugar is sucrose. In some aspects, the lyophilized composition comprises about 0.01 to about 1.0 % w/w of a poloxamer. In some aspects, the poloxamer is poloxamer 188. In some aspects, the lyophilized composition comprises about 1.0 to about 5.0 % w/w of potassium sorbate.

In some aspects, compositions provided herein include an RNA molecule comprising (A) a sequence of SEQ ID NO: 1; (B) a sequence of SEQ ID NO:2; (C) a sequence of SEQ ID NO:3; or (D) a sequence of SEQ ID NO:4. In some aspects, compositions provided herein include an RNA molecule comprising a sequence of SEQ ID NO:29. In some aspects, compositions provided herein include an RNA molecule comprising a sequence of SEQ ID NO:32. In some aspects, compositions provided herein include an RNA molecule comprising a sequence of SEQ ID NO:48. In some aspects, compositions provided herein include an RNA molecule comprising a sequence of SEQ ID NO:40.

Provided herein, in some embodiments, are lipid nanoparticle compositions comprising a. a lipid formulation comprising i. about 45 mol% to about 55 mol% of an ionizable cationic lipid having the structure of ATX-126:

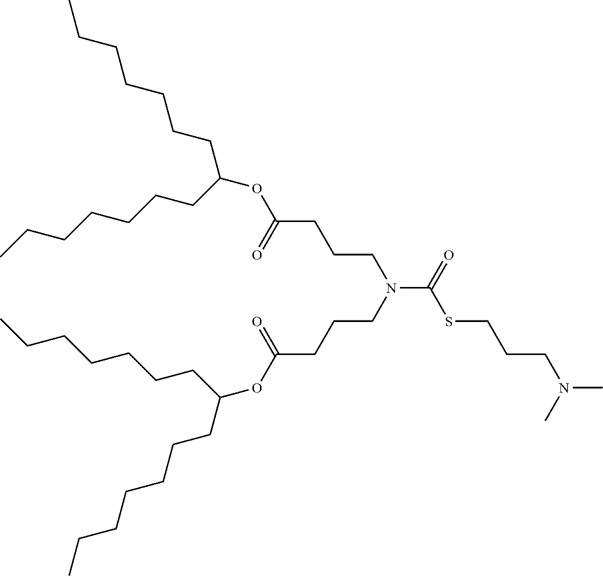 [SMILES: CCCCCCCC(CCCCCCC)OC(=O)CCCN(CCCC(=O)OC(CCCCCCC)CCCCCCC)C(=O)SCCCN(C)C]

ii. about 8 mol% to about 12 mol% DSPC; iii. about 35 mol% to about 42 mol% cholesterol; and iv. about 1.25 mol% to about 1.75 mol% PEG2000-DMG; and b. an RNA molecule having at least 80% identity to a sequence of SEQ ID NO: 1, SEQ ID NO:2, SEQ ID NO:3, or SEQ ID NO:4; wherein the lipid formulation encapsulates the RNA molecule and the lipid nanoparticle has a size of about 60 to about 90 nm. In some aspects, the RNA molecule included in lipid nanoparticle compositions provided herein has at least 80% identity to a sequence of SEQ ID NO:29. In some aspects, the RNA molecule included in lipid nanoparticle compositions provided herein has at least 80% identity to a sequence of SEQ ID NO:32. In some aspects, the RNA molecule included in lipid nanoparticle compositions provided herein has at least 80% identity to a sequence of SEQ ID NO:40. In some aspects, the RNA molecule included in lipid nanoparticle compositions provided herein has at least 80% identity to a sequence of SEQ ID NO:48. In some aspects, the RNA molecule included in lipid nanoparticle compositions provided herein has at least 80% identity to a sequence of SEQ ID NO:29. In some aspects, the RNA molecule included in lipid nanoparticle compositions provided herein has at least 80% identity to a sequence of SEQ ID NO: 32.

Provided herein, in some embodiments, are methods for administering compositions provided herein to a subject in need thereof. In some aspects, compositions provided herein are administered intramuscularly, subcutaneously, intradermally, transdermally, intranasally, orally, sublingually, intravenously, intraperitoneally, topically, by aerosol, or by a pulmonary route. In some aspects, compositions provided herein are administered intramuscularly.

Provided herein, in some embodiments, are methods of administering a composition provided herein to a subject in need thereof, wherein the composition is lyophilized and is reconstituted prior to administration.

Provided herein, in some embodiments, are methods of preventing or ameliorating COVID-19, comprising administering a composition provided herein to a subject in need thereof. In some aspects, the composition is administered one time. In some aspects, the composition is administered two times.

Provided herein, in some embodiments, are methods of administering a booster dose to a vaccinated subject, comprising administering a composition provided herein to a subject who was previously vaccinated against coronavirus.

In some aspects, a composition provided herein is administered at a dosage of about 0.01 µg to about 1,000 µg of nucleic acid in the methods provided herein. In some aspects, a composition provided herein is administered at a dosage of about 1, 2, 5, 7.5, or 10 µg of nucleic acid.

Provided herein, in some embodiments, are methods of inducing an immune response in a subject comprising: administering to the subject an effective amount of an RNA molecule provided herein. In some aspects, the RNA molecule is administered intramuscularly, subcutaneously, intradermally, transdermally, intranasally, orally, sublingually, intravenously, intraperitoneally, topically, by aerosol, or by a pulmonary route.

Provided herein, in some embodiments, are methods of inducing an immune response in a subject comprising: administering to the subject an effective amount of a composition provided herein. In some aspects, the composition is administered intramuscularly, subcutaneously, intradermally, transdermally, intranasally, orally, sublingually, intravenously, intraperitoneally, topically, by aerosol, or by a pulmonary route.

Provided herein, in some embodiments, are RNA molecules for use in inducing an immune response to the first antigenic protein or fragment thereof.

Also provided herein, in some embodiments, is the use of an RNA molecule provided herein in the manufacture of a medicament for inducing an immune response to the first antigenic protein or fragment thereof.

In another embodiment, the present disclosure provides an RNA molecule for expressing an antigen comprising an open reading frame having at least 80% identity to a sequence of SEQ ID NO:33 or SEQ ID NO:30, wherein T is substituted with U.

In some aspects the RNA molecule further comprises a 5′ UTR having a sequence selected from SEQ ID NO:35, SEQ ID NOs: 189-218, or SEQ ID NOs:233-279.

In some aspects the RNA molecule further comprises a 3′ UTR having a sequence selected from SEQ ID NO:37, SEQ ID NOs:219-225, or SEQ ID NOs:280-317.

In some aspects the RNA molecule further comprises a 5′ cap. In some aspects the 5′ cap has a Cap 1 structure, a Cap 1 (m6A) structure, a Cap 2 structure, or a Cap 0 structure.

In some aspects the RNA molecule further comprises a poly-A tail.

In another embodiment, the present disclosure provides an RNA molecule for expressing an antigen comprising an open reading frame having at least 80% identity to a sequence of SEQ ID NO:33, a 5′ UTR comprising a sequence of SEQ ID NO:35, and a 3′ UTR comprising a sequence of SEQ ID NO:37; or an open reading frame having at least 80% identity to a sequence of SEQ ID NO:30, a 5′ UTR comprising a sequence of SEQ ID NO:35, and a 3′ UTR comprising a sequence of SEQ ID NO:37, wherein T is substituted with U.

In some aspects the RNA molecule further comprises a 5′ cap. In some aspects the 5′ cap has a Cap 1 structure, a Cap 1 (m6A) structure, a Cap 2 structure, or a Cap 0 structure.

In some aspects the RNA molecule further comprises a poly-A tail.

In another embodiment, the present disclosure provides a DNA molecule encoding any one of the RNA molecules described herein.

In some aspects the DNA molecule comprises a promoter. In some aspects the promoter is a T7 promoter, a T3 promoter, or an SP6 promoter.

In another embodiment, the present disclosure provides a composition comprising any of the RNA molecules described herein, and a lipid formulation.

In some aspects the lipid formulation is selected from a lipoplex, a liposome, a lipid nanoparticle, a polymer-based carrier, an exosome, a lamellar body, a micelle, and an emulsion.

In some aspects the lipid formulation is a liposome selected from a cationic liposome, a nanoliposome, a proteoliposome, a unilamellar liposome, a multilamellar liposome, a ceramide-containing nanoliposome, and a multivesicular liposome.

In some aspects the lipid formulation is a lipid nanoparticle.

In some aspects the lipid formulation comprises one or more cationic lipids. In some aspects the one or more cationic lipids is selected from 5-carboxyspermylglycinedioctadecylamide (DOGS), 2,3-dioleyloxy-N-[2(spermine-carboxamido)ethyl]-N,N-dimethyl-1-propanaminium (DOSPA), 1,2-Dioleoyl-3-Dimethylammonium-Propane (DODAP), 1,2-Dioleoyl-3-Trimethylammonium-Propane (DOTAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), N-dioleyl-N,N-dimethylammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethy 1-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), and 2,2-dilinoleyl-4-dimethylaminoethyl-[1,3]-dioxolane or (DLin-K-XTC2-DMA).

In some aspects the lipid formulation comprises an ionizable cationic lipid. In some aspects the ionizable cationic lipid has a structure of Formula I:

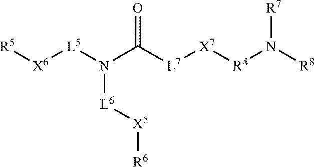

or a pharmaceutically acceptable salt or solvate thereof, wherein R5 and R6 are each independently selected from the group consisting of a linear or branched C1-C31 alkyl, C2-C31 alkenyl or C2-C31 alkynyl and cholesteryl; L5 and L6 are each independently selected from the group consisting of a linear C1-C20 alkyl and C2-C20 alkenyl; X5 is —C(O)O, whereby —C(O)O—R6 is formed or —OC(O)— whereby —OC(O)—R6 is formed; X6 is —C(O)O—whereby —C(O)O—R5 is formed or —OC(O)— whereby —OC(O)—R5 is formed; X7 is S or O; L7 is absent or lower alkyl; R4 is a linear or branched C1-C6 alkyl; and R7 and R8 are each independently selected from the group consisting of a hydrogen and a linear or branched C1-C6 alkyl.

In some aspects the ionizable cationic lipid is selected from

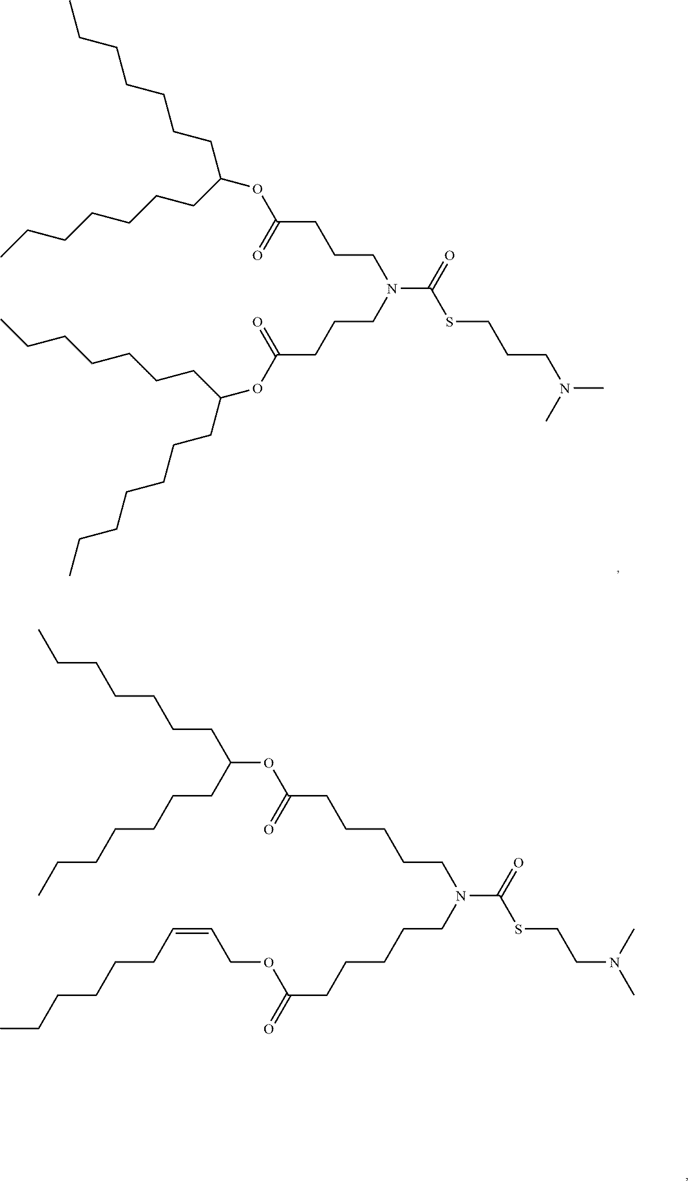

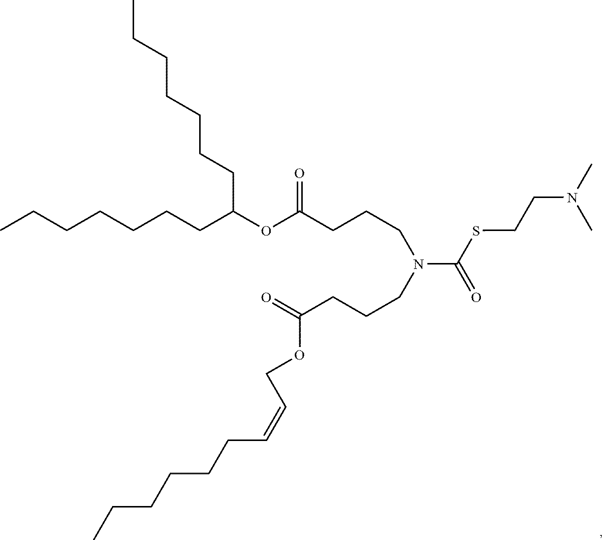

or a pharmaceutically acceptable salt thereof.

In some aspects the lipid formulation comprises a helper lipid. In some aspects the helper lipid is a phospholipid.

In some aspects the helper lipid is selected from dioleoylphosphatidyl ethanolamine (DOPE), dimyristoylphosphatidyl choline (DMPC), distearoylphosphatidyl choline (DSPC), dimyristoylphosphatidyl glycerol (DMPG), dipalmitoyl phosphatidylcholine (DPPC), and phosphatidylcholine (PC).

In some aspects the lipid formulation comprises cholesterol.

In some aspects the lipid formulation comprises a polyethylene glycol (PEG)-lipid conj ugate.

In another embodiment, the present disclosure provides a method of inducing an immune response in a subject comprising administering to the subject an effective amount of any of the RNA molecules or compositions described herein.

In some aspects the method comprises administering the RNA molecule or the composition intramuscularly, subcutaneously, intradermally, transdermally, intranasally, orally, sublingually, intravenously, intraperitoneally, topically, or by a pulmonary route.

In another embodiment, the present disclosure provides a method of administering administering a booster dose to a vaccinated subject, comprising administering any of the RNA molecules or compositions described herein to a subject who was previously vaccinated against coronavirus.

In some aspects the method comprises administering the RNA molecule or the composition intramuscularly, subcutaneously, intradermally, transdermally, intranasally, orally, sublingually, intravenously, intraperitoneally, topically, or by a pulmonary route.

In some aspects the RNA molecules or compositions described herein are used for inducing an immune response to the antigen.

In some aspects the RNA molecules or compositions described herein are used in the manufacture of a medicament for inducing an immune response to the antigen.

### BRIEF DESCRIPTION OF THE DRAWINGS

FIG. 1A shows a schematic of an exemplary self-replicating RNA, including nsP1-nsP4 replicase and coronavirus spike transgene regions.

FIG. 1B shows exemplary miRNA binding sites based on predictions by miRanda (Enright, A.J., John, B., Gaul, U. et al. MicroRNA targets in Drosophila. Genome Biol 5, R1 (2003). doi.org/10.1186/gb-2003-5-1-r1). The Venezuelan equine encephalitis virus (VEEV) non-structural protein coding region is shown, with 15 predicted binding sites shown by grey rectangles.

FIG. 2A shows a Western blot of SARS-CoV-2 spike protein expressed from the indicated construct. Full-length spike protein and the S1 and S2 domains are indicated by arrows.

FIG. 2B shows quantitation of SARS-CoV-2 spike protein expressed from the indicated constructs.

FIG. 3A shows a Western blot of SARS-CoV-2 South African variant spike protein expressed from the indicated construct. The arrow indicates the full-length spike protein.

FIG. 3B shows a Western blot of SARS-CoV-2 D614G variant spike protein expressed from the indicated construct. The arrow indicates the full-length spike protein.

FIG. 3C shows a Western blot of SARS-CoV-2 D614G variant spike protein expressed from the indicated construct. The arrow indicates the full-length spike protein.

FIG. 3D shows quantitation of SARS-CoV-2 spike protein expression from the indicated constructs.

FIG. 4A shows quantitation of SARS-CoV-2 South African variant spike protein expression from the indicated construct as compared to reference.

FIG. 4B shows quantitation of SARS-CoV-2 D614G variant spike protein expression from the indicated construct as compared to reference.

FIG. 4C shows quantitation of SARS-CoV-2 D614G variant spike protein expression from the indicated construct as compared to reference.

FIG. 5A shows total Immunoglobulin G (IgG) against the indicated SARS-CoV-2 spike proteins following immunization of mice with self-replicating RNA encoding a SARS-CoV-2 wild-type spike protein (Wuhan).

FIG. 5B shows neutralizing antibodies against the indicated SARS-CoV-2 spike proteins following immunization of mice with self-replicating RNA encoding a SARS-CoV-2 wild-type spike protein (Wuhan).

FIG. 5C shows total IgG against the indicated SARS-CoV-2 spike protein variants following immunization of mice with self-replicating RNA encoding a SARS-CoV-2 D614G spike protein variant.

FIG. 5D shows neutralizing antibodies against the indicated SARS-CoV-2 spike proteins following immunization of mice with self-replicating RNA encoding a SARS-CoV-2 D614G spike protein variant.

FIG. 5E shows total IgG against the indicated SARS-CoV-2 spike proteins following immunization of mice with self-replicating RNA encoding a SARS-CoV-2 South African spike protein variant.

FIG. 5F shows neutralizing antibodies against the indicated SARS-CoV-2 spike proteins following immunization of mice with self-replicating RNA encoding a SARS-CoV-2 South African spike protein variant.

FIG. 6A shows total IgG against the indicated SARS-CoV-2 spike proteins following immunization of mice with 2 µg of an mRNA RNA encoding a SARS-CoV-2 D614G spike protein variant.

FIG. 6B shows total IgG against the indicated SARS-CoV-2 spike proteins following immunization of mice with 15 µg of an mRNA RNA encoding a SARS-CoV-2 D614G spike protein variant.

FIG. 6C shows neutralizing antibodies against the indicated SARS-CoV-2 spike proteins following immunization of mice with 2 µg of an mRNA RNA encoding a SARS-CoV-2 D614G spike protein variant.

FIG. 6D shows neutralizing antibodies against the indicated SARS-CoV-2 spike proteins following immunization of mice with 15 µg of an mRNA RNA encoding a SARS-CoV-2 D614G spike protein variant.

FIG. 7A shows total IgG against the indicated SARS-CoV-2 spike proteins following immunization of non-human primates (NHPs) with self-replicating RNA encoding a SARS-CoV-2 wild-type spike protein (Wuhan).

FIG. 7B shows neutralizing antibodies against the indicated SARS-CoV-2 spike proteins following immunization of non-human primates (NHPs) with self-replicating RNA encoding a SARS-CoV-2 wild-type spike protein (Wuhan).

FIG. 7C shows total IgG against the indicated SARS-CoV-2 spike proteins following immunization of non-human primates (NHPs) with self-replicating RNA encoding a SARS-CoV-2 D614G spike protein variant.

FIG. 7D shows neutralizing antibodies against the indicated SARS-CoV-2 spike proteins following immunization of non-human primates (NHPs) with self-replicating RNA encoding a SARS-CoV-2 D614G spike protein variant.

FIG. 7E shows total IgG against the indicated SARS-CoV-2 spike proteins following immunization of non-human primates (NHPs) with self-replicating RNA encoding a SARS-CoV-2 South African spike protein variant.

FIG. 7F shows neutralizing antibodies against the indicated SARS-CoV-2 spike proteins following immunization of non-human primates (NHPs) with self-replicating RNA encoding a SARS-CoV-2 South African spike protein variant.

FIG. 7G shows total IgG against the indicated SARS-CoV-2 spike proteins following immunization of non-human primates (NHPs) with an mRNA RNA encoding a SARS-CoV-2 D614G spike protein variant.

FIG. 7H shows neutralizing antibodies against the indicated SARS-CoV-2 spike proteins following immunization of non-human primates (NHPs) with an mRNA RNA encoding a SARS-CoV-2 D614G spike protein variant.

FIG. 8 shows HAI titers obtained for self-replicating RNA and mRNA constructs encoding the hemagglutinin of influenza virus A/California/07/2009 (H1N1).

FIGS. 9A-9D show results of Luminex Assay for anti-SARS-Cov-2 Spike Glycoprotein IgG in two pre-clinical studies. BALB/c mice were vaccinated with increasing RNA doses of self-replicating RNA (SEQ ID NO: 18) formulated as lyophilized lipid nanoparticles (LYO-LNP) and liquid (frozen) lipid nanoparticles (Liquid-LNP). (9A) First Study 0.2 µg, (9B) First Study 2 µg, (9C) Second Study 0.2 µg, and (9D) Second Study 2 µg. Blood was collected and processed to serum at various times post-vaccination and evaluated for anti-SARS-CoV-2 spike glycoprotein IgG. Two way ANOVA, Tukey’s multiple comparison post-test compared LYO-LNP to Liquid-LNP where * p < 0.0332, ** p < 0.0021, *** p < 0.0002, **** p < 0.0001.

FIGS. 10A-10B show the Area Under the Curve (AUC) Analysis for anti-SARS-Cov-2 Spike Glycoprotein IgG (First and Second Study combined data). IgG assay results were combined from two studies to evaluate self-replicating RNA (SEQ ID NO: 18) formulated as lyophilized lipid nanoparticles (LYO-LNP) and liquid (frozen) lipid nanoparticles (Liquid-LNP) at (10A) 0.2 µg, and (10B) 2 µg. N=10/group. First Study Day 19 and 31 results were combined with Second Study Day 20 and 30 results, respectively, and an Area Under the Curve (AUC) analysis was performed. One way ANOVA, Sidak’s multiple comparison post-test compared LYO-LNP to Liquid-LNP and resulted in no statistical differences.

### DETAILED DESCRIPTION

The present disclosure relates to RNAs, e.g., self-replicating RNAs and messenger RNAs (mRNAs), and nucleic acids encoding the same for expression of transgenes such as antigenic proteins, for example. Also provided herein are methods of administration (e.g., to a host, such as a mammalian subject) of RNAs, whereby the RNA is translated in vivo and the heterologous protein-coding sequence is expressed and, e.g., can elicit an immune response to the heterologous protein-coding sequence in the recipient or provide a therapeutic effect, including induction of an immune response, where the heterologous protein-coding sequence is a therapeutic or an antigenic protein. RNAs, e.g., self-replicating RNAs and messenger RNAs (mRNAs), provided herein are useful as vaccines that can be rapidly generated and that can be effective at low and/or single doses. The present disclosure further relates to methods of inducing an immune response using RNAs provided herein.

In some embodiments, an immune response can be elicited against coronavirus. Immunogens include, but are not limited to, those derived from a SARS coronavirus, avian infectious bronchitis (IBV), Mouse hepatitis virus (MHV), and Porcine transmissible gastroenteritis virus (TGEV). The coronavirus immunogen may be a spike polypeptide.

Self-replicating RNAs are described, for example, in U.S. 2018/0036398, the contents of which are incorporated by reference in their entirety.

### Definitions

As used herein, the term “fragment,” when referring to a protein or nucleic acid, for example, means any shorter sequence than the full-length protein or nucleic acid. Accordingly, any sequence of a nucleic acid or protein other than the full-length nucleic acid or protein sequence can be a fragment. In some aspects, a protein fragment includes an epitope. In other aspects, a protein fragment is an epitope.

As used herein, the term “nucleic acid” refers to any deoxyribonucleic acid (DNA) molecule, ribonucleic acid (RNA) molecule, or nucleic acid analogues. A DNA or RNA molecule can be double-stranded or single-stranded and can be of any size. Exemplary nucleic acids include, but are not limited to, chromosomal DNA, plasmid DNA, cDNA, cell-free DNA (cfDNA), mitochondrial DNA, chloroplast DNA, viral DNA, mRNA, tRNA, rRNA, long non-coding RNA, siRNA, micro RNA (miRNA or miR), hnRNA, and viral RNA. Exemplary nucleic analogues include peptide nucleic acid, morpholino- and locked nucleic acid, glycol nucleic acid, and threose nucleic acid. As used herein, the term “nucleic acid molecule” is meant to include fragments of nucleic acid molecules as well as any full-length or non-fragmented nucleic acid molecule, for example. As used herein, the terms “nucleic acid” and “nucleic acid molecule” can be used interchangeably, unless context clearly indicates otherwise.

As used herein, the term “polynucleotide” refers to a nucleic acid sequence that includes at least two nucleotide monomers. The term “polynucleotide” can refer to DNA, RNA, or nucleic acid analogues. A “polynucleotide” can be double-stranded or single-stranded and can be of any size. A polynucleotide can be a separate nucleic acid molecule or be a part of a nucleic acid molecule. Accordingly, the term “polynucleotide” can refer to a nucleic acid molecule or to a region of a nucleic acid molecule.

As used herein, the term “protein” refers to any polymeric chain of amino acids. The terms “peptide” and “polypeptide” can be used interchangeably with the term protein, unless context clearly indicates otherwise, and can also refer to a polymeric chain of amino acids. The term “protein” encompasses native or artificial proteins, protein fragments and polypeptide analogs of a protein sequence. A protein may be monomeric or polymeric. The term “protein” encompasses fragments and variants (including fragments of variants) thereof, unless otherwise contradicted by context.

In general, “sequence identity” or “sequence homology,” which can be used interchangeably, refer to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby or the amino acid sequence of a polypeptide, and comparing these sequences to a second nucleotide or amino acid sequence. As used herein, the term “percent (%) sequence identity” or “percent (%) identity,” also including “percent homology,” refers to the percentage of amino acid residues or nucleotides in a sequence that are identical with the amino acid residues or nucleotides in a reference sequence after aligning the sequences and introducing gaps, if necessary, to achieve the maximum percent sequence identity, and not considering any conservative substitutions as part of the sequence identity. Thus, two or more sequences (polynucleotide or amino acid) can be compared by determining their “percent identity,” also referred to as “percent homology.” The percent identity to a reference sequence (e.g., nucleic acid or amino acid sequences), which may be a sequence within a longer molecule (e.g., polynucleotide or polypeptide), may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul et al., J. Mol. Biol. 215:403-410 (1990); Karlin and Altschul, Proc. Natl. Acad. sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17: 149-163 (1993). Ranges of desired degrees of sequence identity are approximately 80% to 100% and integer values in between. Percent identities between a reference sequence and a claimed sequence can be at least 80%, at least 85%, at least 90%, at least 95%, at least 98%, at least 99%, at least 99.5%, or at least 99.9%. In general, an exact match indicates 100% identity over the length of the reference sequence. Additional programs and methods for comparing sequences and/or assessing sequence identity include the Needleman-Wunsch algorithm (see, e.g., the EMBOSS Needle aligner available at ebi.ac.uk/Tools/psa/emboss needle/, optionally with default settings), the Smith-Waterman algorithm (see, e.g., the EMBOSS Water aligner available at ebi.ac.uk/Tools/psa/emboss water/, optionally with default settings), the similarity search method of Pearson and Lipman, 1988, Proc. Natl. Acad. Sci. USA 85, 2444, or computer programs which use these algorithms (GAP, BESTFIT, FASTA, BLAST P, BLAST N and TFASTA in Wisconsin Genetics Software Package, Genetics Computer Group. 575 Science Drive, Madison, Wis.). In some aspects, reference to percent sequence identity refers to sequence identity as measured using BLAST (Basic Local Alignment Search Tool). In other aspects, ClustalW is used for multiple sequence alignment. Optimal alignment may be assessed using any suitable parameters of a chosen algorithm, including default parameters.

As used herein, “homologous sequences” refers to sequences that share sequence similarity and/or structural similarity (Pearson, 2013, An Introduction to Sequence similarity (“Homology”) Searching, Current Protoc Bioinformatics, 42:3.1.1-3.1.8). Accordingly, homologous sequences share common evolutionary ancestry or are derived from a common sequence. Homologous sequences can also share structural or sequence similarity to an intermediate sequence. Homologous sequences can have similar functions, i.e., have functional similarity. Homology can be inferred based on nucleic acid and/or amino acid sequence, with protein similarity searches generally having greater sensitivity than nucleic acid sequence searches. Homology can also be inferred for amino acid sequences that include similar amino acids, i.e., amino acids with similar physiochemical properties, rather than identical amino acids over at least a region of sequence. The terms “homologous sequences,” “homologues,” and “homologous nucleic acid” and/or “homologous protein” can be used interchangeably, unless context clearly indicated otherwise.

As used herein, the term “drug” or “medicament,” means a pharmaceutical formulation or composition as described herein.

As used herein, the singular forms “a,” “an,” and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of +20%, or ±10%, or ±5%, or even ±1% from the specified value, as such variations are appropriate for the disclosed methods or to perform the disclosed methods.

The term “expression” refers to the process by which a nucleic acid sequence or a polynucleotide is transcribed from a DNA template (such as into mRNA or other RNA transcript) and/or the process by which a transcribed mRNA or other RNA is subsequently translated into peptides, polypeptides, or proteins. Transcripts and encoded polypeptides may be collectively referred to as “gene product.”

As used herein, the terms “self-replicating RNA,” “self-transcribing and self-replicating RNA,” “self-amplifying RNA (saRNA),” and “replicon” may be used interchangeably, unless context clearly indicates otherwise. Generally, the term “replicon” or “viral replicon” refers to a self-replicating subgenomic RNA derived from a viral genome that includes viral genes encoding non-structural proteins important for viral replication and that lacks viral genes encoding structural proteins. A self-replicating RNA can encode further subgenomic RNAs that are not able to self-replicate. A self-replicating RNA can also be referred to as a “STARR™” RNA.

As used herein, “operably linked,” “operable linkage,” “operatively linked,” or grammatical equivalents thereof refer to juxtaposition of genetic elements, e.g., a promoter, an enhancer, a polyadenylation sequence, etc., wherein the elements are in a relationship permitting them to operate in the expected manner. For instance, a regulatory element, which can comprise promoter and/or enhancer sequences, is operatively linked to a coding region if the regulatory element helps initiate transcription of the coding sequence. There may be intervening residues between the regulatory element and coding region so long as this functional relationship is maintained.

### RNA Molecules

In some embodiments, provided herein are RNA molecules comprising: (a) a first polynucleotide encoding one or more viral replication proteins, wherein one or more miRNA binding sites in the first polynucleotide have been modified as compared to a reference polynucleotide; and (b) a second polynucleotide comprising a first transgene encoding an antigenic protein or a fragment thereof.

Also provided herein, in some embodiments, are RNA molecules comprising: (i) a first polynucleotide comprising a sequence having at least 80% identity to a sequence of SEQ ID NO:6; and (ii) a second polynucleotide comprising a first transgene encoding a first antigenic protein or a fragment thereof.

Also provided herein, in some embodiments, are RNA molecules for expressing an antigen comprising an open reading frame having at least 80% identity to a sequence of SEQ ID NO:33 or SEQ ID NO:30, wherein T is substituted with U.

Also provided herein are RNA molecules for expressing an antigen comprising an open reading frame having at least 80% identity to a sequence of SEQ ID NO:33, a 5′ UTR comprising a sequence of SEQ ID NO:35, and a 3′ UTR comprising a sequence of SEQ ID NO:37; or an open reading frame having at least 80% identity to a sequence of SEQ ID NO:30, a 5′ UTR comprising a sequence of SEQ ID NO:35, and a 3′ UTR comprising a sequence of SEQ ID NO:37, wherein T is substituted with U.

An RNA molecule can encode a single polypeptide immunogen or multiple polypeptides. Multiple immunogens can be presented as a single polypeptide immunogen (fusion polypeptide) or as separate polypeptides. If immunogens are expressed as separate polypeptides from a replicon then one or more of these may be provided with an upstream IRES or an additional viral promoter element. Alternatively, multiple immunogens may be expressed from a polyprotein that encodes individual immunogens fused to a short autocatalytic protease (e.g. foot-and-mouth disease virus 2A protein), or as inteins.

### Codon Optimization

In some embodiments, first polynucleotides of RNA molecules provided herein encoding one or more viral replication proteins include codon-optimized sequences. As used herein, the term “codon-optimized” means a polynucleotide, nucleic acid sequence, or coding sequence has been redesigned as compared to a wild-type or reference polynucleotide, nucleic acid sequence, or coding sequence by choosing different codons without altering the amino acid sequence of the encoded protein. Accordingly, codon-optimization generally refers to replacement of codons with synonymous codons to optimize expression of a protein while keeping the amino acid sequence of the translated protein the same. Codon optimization of a sequence can increase protein expression levels (Gustafsson et al., Codon bias and heterologous protein expression. 2004, Trends Biotechnol 22: 346-53) of the encoded proteins, for example, and provide other advantages. Variables such as codon usage preference as measured by codon adaptation index (CAI), for example, the presence or frequency of U and other nucleotides, mRNA secondary structures, cis-regulatory sequences, GC content, and other variables may correlate with protein expression levels (Villalobos et al., Gene Designer: a synthetic biology tool for constructing artificial DNA segments. 2006, BMC Bioinformatics 7:285). First polynucleotides can be codon-optimized before modifying miRNA binding sites. miRNA binding sites can be modified to replace one or more codons with synonymous codons.

Any method of codon optimization can be used to codon optimize polynucleotides and nucleic acid molecules provided herein, and any variable can be altered by codon optimization. Accordingly, any combination of codon optimization methods can be used. Exemplary methods include the high codon adaptation index (CAI) method, the Low U method, and others. The CAI method chooses a most frequently used synonymous codon for an entire protein coding sequence. As an example, the most frequently used codon for each amino acid can be deduced from 74,218 protein-coding genes from a human genome. The Low U method targets U-containing codons that can be replaced with a synonymous codon with fewer U moieties, generally without changing other codons. If there is more than one choice for replacement, the more frequently used codon can be selected. Any polynucleotide, nucleic acid sequence, or codon sequence provided herein can be codon-optimized.

In some embodiments, the nucleotide sequence of any region of the RNA or DNA templates described herein may be codon optimized. Preferably, the primary cDNA template may include reducing the occurrence or frequency of appearance of certain nucleotides in the template strand. For example, the occurrence of a nucleotide in a template may be reduced to a level below 25% of said nucleotides in the template. In further examples, the occurrence of a nucleotide in a template may be reduced to a level below 20% of said nucleotides in the template. In some examples, the occurrence of a nucleotide in a template may be reduced to a level below 16% of said nucleotides in the template. Preferably, the occurrence of a nucleotide in a template may be reduced to a level below 15%, and preferably may be reduced to a level below 12% of said nucleotides in the template.

In some embodiments, the nucleotide reduced is uridine. For example, the present disclosure provides nucleic acids with altered uracil content wherein at least one codon in the wild-type sequence has been replaced with an alternative codon to generate a uracil-altered sequence. Altered uracil sequences can have at least one of the following properties:
(i) an increase or decrease in global uracil content (i.e., the percentage of uracil of the total nucleotide content in the nucleic acid of a section of the nucleic acid, e.g., the open reading frame);
(ii) an increase or decrease in local uracil content (i.e., changes in uracil content are limited to specific subsequences);
(iii) a change in uracil distribution without a change in the global uracil content;
(iv) a change in uracil clustering (e.g., number of clusters, location of clusters, or distance between clusters); or
(v) combinations thereof.

In some embodiments, the percentage of uracil nucleobases in the nucleic acid sequence is reduced with respect to the percentage of uracil nucleobases in the wild-type nucleic acid sequence. For example, 30% of nucleobases may be uracil in the wild-type sequence but the nucleobases that are uracil are preferably lower than 15%, preferably lower than 12% and preferably lower than 10% of the nucleobases in the nucleic acid sequences of the disclosure. The percentage uracil content can be determined by dividing the number of uracil in a sequence by the total number of nucleotides and multiplying by 100.

In some embodiments, the percentage of uracil nucleobases in a subsequence of the nucleic acid sequence is reduced with respect to the percentage of uracil nucleobases in the corresponding subsequence of the wild-type sequence. For example, the wild-type sequence may have a 5′-end region (e.g., 30 codons) with a local uracil content of 30%, and the uracil content in that same region could be reduced to preferably 15% or lower, preferably 12% or lower and preferably 10% or lower in the nucleic acid sequences of the disclosure. These subsequences can also be part of the wild-type sequences of the heterologous 5′ and 3′ UTR sequences of the present disclosure.

In some embodiments, codons in the nucleic acid sequence of the disclosure reduce or modify, for example, the number, size, location, or distribution of uracil clusters that could have deleterious effects on protein translation. Although lower uracil content is desirable in certain aspects, the uracil content, and in particular the local uracil content, of some subsequences of the wild-type sequence can be greater than the wild-type sequence and still maintain beneficial features (e.g., increased expression).

In some embodiments, the uracil-modified sequence induces a lower Toll-Like Receptor (TLR) response when compared to the wild-type sequence. Several TLRs recognize and respond to nucleic acids. Double-stranded (ds)RNA, a frequent viral constituent, has been shown to activate TLR3. Single-stranded (ss)RNA activates TLR7. RNA oligonucleotides, for example RNA with phosphorothioate internucleotide linkages, are ligands of human TLR8. DNA containing unmethylated CpG motifs, characteristic of bacterial and viral DNA, activate TLR9.

As used herein, the term “TLR response” is defined as the recognition of single-stranded RNA by a TLR7 receptor, and preferably encompasses the degradation of the RNA and/or physiological responses caused by the recognition of the single-stranded RNA by the receptor. Methods to determine and quantify the binding of an RNA to a TLR7 are known in the art. Similarly, methods to determine whether an RNA has triggered a TLR7-mediated physiological response (e.g., cytokine secretion) are well known in the art. In some embodiments, a TLR response can be mediated by TLR3, TLR8, or TLR9 instead of TLR7. Suppression of TLR7-mediated response can be accomplished via nucleoside modification. RNA undergoes over a hundred different nucleoside modifications in nature. Human rRNA, for example, has ten times more pseudouracil (′P) and 25 times more 2′-O-methylated nucleosides than bacterial rRNA. Bacterial RNA contains no nucleoside modifications, whereas mammalian RNAs have modified nucleosides such as 5-methylcytidine (m5C), N6-methyladenosine (m6A), inosine and many 2′-O-methylated nucleosides in addition to N7-methylguanosine (m7G).

In some embodiments, the uracil content of polynucleotides disclosed herein is less than about 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% of the total nucleobases in the sequence in the reference sequence. In some embodiments, the uracil content of polynucleotides disclosed herein is between about 5% and about 25%. In some embodiments, the uracil content of polynucleotides disclosed herein is between about 15% and about 25%.

In some embodiments, the nucleotide that is increased or decreased is a nucleotide other than or in addition to uracil. Sequences with altered nucleotide content can have (i) an increase or decrease in local C content (i.e., changes in cytosine content are limited to specific subsequences); (ii) an increase or decrease in local G content (i.e., changes in guanosine content are limited to specific subsequences); or (iii) a combination thereof.

In some embodiments, first polynucleotides of nucleic acid molecules provided herein comprise a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:6. In some embodiments, first polynucleotides of nucleic acid molecules provided herein comprise a sequence of SEQ ID NO:6.

### Intergenic Region

In some aspects, first polynucleotides and second polynucleotides of nucleic acid molecules provided herein are included in the same (i.e., a single) or in separate nucleic acid molecules. Generally, first polynucleotides and second polynucleotides of nucleic acid molecules provided herein are included in a single nucleic acid molecule. In one aspect, the first polynucleotide is located 5′ of the second polynucleotide. In one aspect, first polynucleotides and second polynucleotides of nucleic acid molecules provided herein are included in separate nucleic acid molecules. In yet another aspect, first polynucleotides and second polynucleotides are included in two separate nucleic acid molecules.

In some aspects, first polynucleotides and second polynucleotides are included in the same (i.e., a single) nucleic acid molecule. First polynucleotides and second polynucleotides of nucleic acid molecules provided herein can be contiguous, i.e., adjacent to each other without nucleotides in between. In one aspect, an intergenic region is located between the first polynucleotide and the second polynucleotide. As used herein, the terms “intergenic region” and intergenic sequence” can be used interchangeably, unless context clearly indicates otherwise.

An intergenic region located between the first polynucleotide and the second polynucleotide can be of any length and can have any nucleotide sequence. As an example, the intergenic region between the first polynucleotide and the second polynucleotide can include about one nucleotide, about two nucleotides, about three nucleotides, about four nucleotides, about five nucleotides, about six nucleotides, about seven nucleotides, about eight nucleotides, about nine nucleotides, about ten nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides, about 60 nucleotides, about 70 nucleotides, about 80 nucleotides, about 90 nucleotides, about 100 nucleotides, about 125 nucleotides, about 150 nucleotides, about 175 nucleotides, about 200 nucleotides, about 250 nucleotides, about 300 nucleotides, about 350 nucleotides, about 400 nucleotides, about 450 nucleotides, about 500 nucleotides, about 600 nucleotides, about 700 nucleotides, about 800 nucleotides, about 900, about 1,000 nucleotides, about 1,500 nucleotides, about 2,000 nucleotides, about 2,500 nucleotides, about 3,000 nucleotides, about 3,500 nucleotides, about 4,000 nucleotides, about 4,500 nucleotides, about 5,000 nucleotides, about 6,000 nucleotides, about 7,000 nucleotides, about 8,000 nucleotides, about 9,000 nucleotides, about 10,000 nucleotides, and any number or range in between. In one aspect, the intergenic region between first and second polynucleotides includes about 10-100 nucleotides, about 10-200 nucleotides, about 10-300 nucleotides, about 10-400 nucleotides, or about 10-500 nucleotides. In another aspect, the intergenic region between first and second polynucleotides includes about 1-10 nucleotides, about 1-20 nucleotides, about 1-30 nucleotides, about 1-40 nucleotides, or about 1- 50 nucleotides. In yet another aspect, the region includes about 44 nucleotides.

In one aspect, the intergenic region between first and second polynucleotides includes a viral sequence. The intergenic region between first and second polynucleotides can include a sequence from any virus, such as alphaviruses and rubiviruses, for example. In one aspect, the intergenic region between the first polynucleotide and the second polynucleotide comprises an alphavirus sequence, such as a sequence from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O’Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), Buggy Creek Virus (BCRV), or any combination thereof. In another aspect, the intergenic region between first and second polynucleotides comprises a sequence from Venezuelan Equine Encephalitis Virus (VEEV). In yet another aspect, the intergenic region between first and second polynucleotides comprises a sequence having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:7. In a further aspect, the intergenic region between first and second polynucleotides comprises a sequence of SEQ ID NO:7. In yet a further aspect, the intergenic region between first and second polynucleotides is a second intergenic region comprising a sequence having at least 85% identity to a sequence of SEQ ID NO:7.

### Natural and Modified Nucleotides

A self-replicating RNA of the disclosure can comprise one or more chemically modified nucleotides. Examples of nucleic acid monomers include non-natural, modified, and chemically-modified nucleotides, including any such nucleotides known in the art. Nucleotides can be artificially modified at either the base portion or the sugar portion. In nature, most polynucleotides comprise nucleotides that are “unmodified” or “natural” nucleotides, which include the purine bases adenine (A) and guanine (G), and the pyrimidine bases thymine (T), cytosine (C) and uracil (U). These bases are typically fixed to a ribose or deoxy ribose at the 1′ position. The use of RNA polynucleotides comprising chemically modified nucleotides have been shown to improve RNA expression, expression rates, half-life and/or expressed protein concentrations. RNA polynucleotides comprising chemically modified nucleotides have also been useful in optimizing protein localization thereby avoiding deleterious bio-responses such as immune responses and/or degradation pathways.

Examples of modified or chemically-modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N4-alkylcytidines, N4-aminocytidines, N4-acetylcytidines, and N4,N4-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N4-methylcytidine, N4-aminocytidine, N4-acetylcytidine, and N4,N4-dimethylcytidine.

Examples of modified or chemically-modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically-modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “5MeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2’O-methyluridine, and 3,2′-O-dimethyluridine.

Examples of modified or chemically-modified nucleotides include N6-methyladenosine, 2-aminoadenosine, 3-methyladenosine, 8-azaadenosine, 7-deazaadenosine, 8-oxoadenosine, 8-bromoadenosine, 2-methylthio-N6-methyladenosine, N6-isopentenyladenosine, 2-methylthio-N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyl-adenosine, N6-methyl-N6-threonylcarbamoyl-adenosine, 2-methylthio-N6-threonylcarbamoyl-adenosine, N6,N6-dimethyladenosine, N6-hydroxynorvalylcarbamoyladenosine, 2-methylthio-N6-hydroxynorvalylcarbamoyl-adenosine, N6-acetyl-adenosine, 7-methyl-adenine, 2-methylthio-adenine, 2-methoxy-adenine, alpha-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine, 2-amino-N6-methyl-purine, 1-thio-adenosine, 2′-F-ara-adenosine, 2′-F-adenosine, 2′-OH-ara-adenosine, and N6-(19-amino-pentaoxanonadecyl)-adenosine.

Examples of modified or chemically-modified nucleotides include Nl-alkylguanosines, N2-alkylguanosines, thienoguanosines, 7-deazaguanosines, 8-oxoguanosines, 8-bromoguanosines, O6-alkylguanosines, xanthosines, inosines, and Nl-alkylinosines.

Examples of modified or chemically-modified nucleotides include Nl-methylguanosine, N2-methylguanosine, thienoguanosine, 7-deazaguanosine, 8-oxoguanosine, 8-bromoguanosine, O6-methylguanosine, xanthosine, inosine, and Nl-methylinosine.

Examples of modified or chemically-modified nucleotides include pseudouridines. Examples of pseudouridines include Nl-alkylpseudouridines, Nl-cycloalkylpseudouridines, N1-hydroxypseudouridines, N1-hydroxyalkylpseudouridines, Nl-phenylpseudouridines, Nl-phenylalkylpseudouridines, Nl-aminoalkylpseudouridines, N3-alkylpseudouridines, N6-alkylpseudouridines, N6-alkoxypseudouridines, N6-hydroxypseudouridines, N6-hydroxyalkylpseudouridines, N6-morpholinopseudouridines, N6-phenylpseudouridines, and N6-halopseudouridines. Examples of pseudouridines include Nl-alkyl-N6-alkylpseudouridines, Nl-alkyl-N6-alkoxypseudouridines, Nl-alkyl-N6-hydroxypseudouridines, Nl-alkyl-N6-hydroxyalkylpseudouridines, Nl-alkyl-N6-morpholinopseudouridines, Nl-alkyl-N6-phenylpseudouridines, and Nl-alkyl-N6-halopseudouridines. In these examples, the alkyl, cycloalkyl, and phenyl substituents may be unsubstituted, or further substituted with alkyl, halo, haloalkyl, amino, or nitro substituents.

Examples of pseudouridines include Nl-methylpseudouridine (also referred to herein as “NIMPU”), Nl-ethylpseudouridine, Nl-propylpseudouridine, Nl-cyclopropylpseudouridine, Nl-phenylpseudouridine, Nl-aminomethylpseudouridine, N3-methylpseudouridine, N1-hydroxypseudouridine, and N1-hydroxymethylpseudouridine.

Examples of nucleic acid monomers include modified and chemically-modified nucleotides, including any such nucleotides known in the art.

Examples of modified and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically-modified nucleotide monomers include 3′-end stabilized nucleotides, 3′-glyceryl nucleotides, 3′-inverted abasic nucleotides, and 3′-inverted thymidine.

Examples of modified and chemically-modified nucleotide monomers include locked nucleic acid nucleotides (LNA), 2′-O,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an exemplary embodiment, the modified monomer is a locked nucleic acid nucleotide (LNA).

Examples of modified and chemically-modified nucleotide monomers include 2′,4′-constrained 2′-O-methoxyethyl (cMOE) and 2′-O-Ethyl (cEt) modified DNAs.

Examples of modified and chemically-modified nucleotide monomers include 2′-amino nucleotides, 2′-O-amino nucleotides, 2′-C-allyl nucleotides, and 2′-O-allyl nucleotides.

Examples of modified and chemically-modified nucleotide monomers include N6-methyladenosine nucleotides.

Examples of modified and chemically-modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically-modified nucleotide monomers include 2′-O-aminopropyl substituted nucleotides.

Examples of modified and chemically-modified nucleotide monomers include replacing the 2′—OH group of a nucleotide with a 2′—R, a 2′—OR, a 2′-halogen, a 2′—SR, or a 2′-amino, where R can be H, alkyl, alkenyl, or alkynyl.

Exemplary base modifications described above can be combined with additional modifications of nucleoside or nucleotide structure, including sugar modifications and linkage modifications. Certain modified or chemically-modified nucleotide monomers may be found in nature.

Preferred nucleotide modifications include N1-methylpseudouridine and 5-methoxyuridine.

### Viral Replication Proteins and Polynucleotides Encoding Them

Provided herein, in some embodiments, are RNA molecules comprising a first polynucleotide encoding one or more viral replication proteins. As used herein, the term “replication protein” or “viral replication protein” refers to any protein or any protein subunit of a protein complex that functions in replication of a viral genome. Generally, viral replication proteins are non-structural proteins. Viral replication proteins encoded by nucleic acid molecules provided herein can function in the replication of any viral genome. The viral genome can be a single-stranded positive-sense RNA genome, a single-stranded negative-sense RNA genome, a double-stranded RNA genome, a single-stranded positive-sense DNA genome, a single-stranded negative-sense DNA genome, or a double-stranded DNA genome. Viral genomes can include a single nucleic acid molecule or more than one nucleic acid molecule. Nucleic acid molecules provided herein can encode one or more viral replication proteins from any virus or virus family, including animal viruses and plant viruses, for example. Viral replication proteins encoded by first polynucleotides included in nucleic acid molecules provided herein can be expressed from self-replicating RNA.

In some aspects, first polynucleotides of RNA molecules provided herein include modifications or mutations of one or more microRNA (miRNA; miR) binding sites. In other aspects, modification or mutation of miRNA binding sites reduces or eliminates miRNA binding. In some aspects, miRNA binding is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, and any number or range in between. In some aspects, miRNA binding is reduced by 100%, i.e., there is no miRNA binding. In still other aspects, one, two, three, four, five, six, seven, eight, nine, ten, 11, 12, 13, 14, or 15 miRNA binding sites are modified or mutated.

miRNAs are small single-stranded non-coding RNA molecules that function in RNA silencing and post-transcriptional regulation of gene expression. For example, binding of miRNA to a miRNA binding site in a transcript or messenger RNA (mRNA) can inhibit translation. miRNAs can be found in many eukaryotic cells, including in mammals and plants. Some viruses also produce miRNAs. Generally, miRNAs are produced from larger pri-miRNA molecules that form hairpin loop structures with double-stranded regions. Pri-miRNAs are processed to pre-miRNAs in the nucleus and exported to the cytoplasm. Pre-miRNA hairpins are cleaved in the cytoplasm by the RNase III enzyme Dicer, with one miRNA strand being incorporated into the RNA-induced silencing complex (RISC) and interacting with an mRNA target. In animal cells, miRNAs can recognize a target mRNA via a seed region at the 5′ end of the miRNA that can include as few as 6-8 nucleotides of the miRNA. Binding of miRNA to a target mRNA can result in cleavage of the mRNA in the case of perfect or near-perfect pairing or inhibition of translation without mRNA cleavage. Putative miRNA binding sites can be identified using algorithms, such as miRanda (Enright, A.J., John, B., Gaul, U. et al. MicroRNA targets in Drosophila. Genome Biol 5, R1 (2003). doi.org/10.1186/gb-2003-5-1-r1).

Any modification or mutation can be made in identified or putative miRNA binding sites, including point mutations or substitutions, insertions, and deletions. In some aspects, modifications or mutations of miRNA binding sites include point mutations. More than one nucleotide can be changed in identified or putative miRNA binding sites, including one, two, three, four, five, six, seven, eight, nine, ten, or more nucleotides. In one aspect, point mutations include synonymous nucleotide changes, i.e., changes that do not alter an encoded amino acid. Binding sites for any miRNA provided herein can be modified or mutated. In some aspects, miRNA binding sites that are modified or mutated in first polynucleotides of RNA molecules provided herein are selected from regions that bind a miRNA having a sequence of SEQ ID NOs:58, 59, 72, 80, 81, 83, 101, 102, 103, 112, 113, 114, 128, 131, 142, 156, 157, 171, 175, and any combination thereof.

In some aspects, binding of any miRNA or any combination of miRNAs is reduced by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, and any number or range in between. In some aspects, miRNA binding is reduced by 100%, i.e., there is no miRNA binding. In some aspects, reduction of miRNA binding increases protein expression. Protein expression can be increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, or more, and any number or range in between. In some aspects, protein expression is increased by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8%, about 99.9%, about 100%, about 150%, about 200%, about 250%, about 300%, about 350%, about 400%, about 450%, about 500%, about 550%, about 600%, about 650%, about 700%, about 750%, about 800%, about 850%, about 900%, about 950%, about 1000%, or more, and any number or range in between. Protein expression can also be increased about 1-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 150-fold, about 200-fold, about 250-fold, about 300-fold, about 350-fold, about 400-fold, about 450-fold, about 500-fold, about 600-fold, about 700-fold, about 800-fold, about 900-fold, about 1000-fold, or more, and any number or range in between. In some aspects, protein expression is increased at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 150-fold, at least about 200-fold, at least about 250-fold, at least about 300-fold, at least about 350-fold, at least about 400-fold, at least about 450-fold, at least about 500-fold, at least about 600-fold, at least about 700-fold, at least about 800-fold, at least about 900-fold, at least about 1000-fold, or more, and any number or range in between.

First polynucleotide sequences of RNA molecules provided herein can encode one or more togavirus replication proteins. In some aspects, the one or more viral replication proteins encoded by first polynucleotides of RNA molecules provided herein are alphavirus proteins. In some embodiments, the one or more viral replication proteins encoded by first polynucleotides of RNA molecules provided herein are rubivirus proteins. First polynucleotide sequences of RNA molecules provided herein can encode any alphavirus replication protein and any rubivirus replication protein. Exemplary replication proteins from alphaviruses include proteins from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O’Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), Buggy Creek Virus (BCRV), and any combination thereof. Exemplary rubivirus replication proteins include proteins from rubella virus.

Viral replication proteins encoded by first polynucleotides of RNA molecules provided herein can be expressed as one or more polyproteins or as separate or single proteins. Generally, polyproteins are precursor proteins that are cleaved to generate individual or separate proteins. Accordingly, proteins derived from a precursor polyprotein can be expressed from a single open reading frame (ORF). As used herein, the term “ORF” refers to a nucleotide sequence that begins with a start codon, generally ATG, and that ends with a stop codon, such as TAA, TAG, or TGA, for example. It will be appreciated that T is present in DNA, while U is present in RNA. Accordingly, a start codon of ATG in DNA corresponds to AUG in RNA, and the stop codons TAA, TAG, and TGA in DNA correspond to UAA, UAG, and UGA in RNA. It will further be appreciated that for any sequence provided in the present disclosure, T is present in DNA, while U is present in RNA. Accordingly, for any sequence provided herein, T present in DNA is substituted with U for an RNA molecule, and U present in RNA is substituted with T for a DNA molecule.

The protease cleaving a polyprotein can be a viral protease or a cellular protease. In some aspects, the first polynucleotide of RNA molecules provided herein encodes a polyprotein comprising an alphavirus nsP1 protein, an alphavirus nsP2 protein, an alphavirus nsP3 protein, an alphavirus nsP4 protein, or any combination thereof. In other aspects, the first polynucleotide of RNA molecules provided herein encodes a polyprotein comprising an alphavirus nsP1 protein, an alphavirus nsP2 protein, an alphavirus nsP3 protein, or any combination thereof, and an alphavirus nsP4 protein. In some aspects, the polyprotein is a VEEV polyprotein. In other aspects, the alphavirus nsP1, nsP2, nsP3, and nsP4 proteins are VEEV proteins.

In one aspect, first polynucleotides of RNA molecules provided herein lack a stop codon between sequences encoding an nsP3 protein and an nsP4 protein. Accordingly, in some aspects, first polynucleotides of RNA molecules provided herein encode a P1234 polyprotein comprising nsP1, nsP2, nsP3, and nsP4. First polynucleotides of RNA molecules provided herein can also include a stop codon between sequences encoding an nsP3 and an nsP4 protein. Accordingly, in some aspects, first polynucleotides of nucleic acid molecules provided herein encode a P123 polyprotein comprising nsP1, nsP2, and nsP3 and a P1234 polyprotein comprising nsP1, nsP2, nsP3, and nsP4 as a result of stop codon readthrough, for example. In other aspects, first polynucleotides of RNA molecules provided herein encode a polyprotein having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO: 187. In some embodiments, first polynucleotides of nucleic acid molecules provided herein encode a polyprotein having a sequence of SEQ ID NO:187. In one aspect, nsP2 and nsP3 proteins include mutations. Exemplary mutations include G1309R and S1583G mutations of VEEV proteins. In another aspect, the nsP1, nsP2, and nsP4 proteins are VEEV proteins, and the nsP3 protein is a chikungunya virus (CHIKV) nsP3 protein.

In some embodiments, the first polynucleotide comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, or at least 99.9% identity to a sequence of SEQ ID NO:6. In some embodiments, the first polynucleotide comprises a sequence of SEQ ID NO:6. In some embodiments, the first polynucleotide comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, or at least 99.9% identity to a sequence of SEQ ID NO:42. In some embodiments, the first polynucleotide comprises a sequence of SEQ ID NO:42.

### 5′ Untranslated Region (5′ UTR)

Nucleic acid molecules provided herein can further comprise untranslated regions (UTRs). Untranslated regions, including 5′ UTRs and 3′ UTRs, for example, can affect RNA stability and/or efficiency of RNA translation, such as translation of cellular and viral mRNAs, for example. 5′ UTRs and 3′ UTRs can also affect stability and translation of viral genomic RNAs and self-replicating RNAs, including virally derived self-replicating RNAs or replicons. Exemplary viral genomic RNAs whose stability and/or efficiency of translation can be affected by 5′ UTRs and 3′ UTRs include the genome nucleic acid of positive-sense RNA viruses. Both genome nucleic acid of positive-sense RNA viruses and self-replicating RNAs, including virally derived self-replicating RNAs or replicons, can be translated upon infection or introduction into a cell.

In some aspects, nucleic acid molecules provided herein further include a 5′ untranslated region (5′ UTR). Any 5′ UTR sequence can be included in nucleic acid molecules provided herein. In some embodiments, nucleic acid molecules provided herein include a viral 5′ UTR. In one aspect, nucleic acid molecules provided herein include a non-viral 5′ UTR. Any non-viral 5′ UTR can be included in nucleic acid molecules provided herein, such as 5′ UTRs of transcripts expressed in any cell or organ, including muscle, skin, subcutaneous tissue, liver, spleen, lymph nodes, antigen-presenting cells, and others. In another aspect, nucleic acid molecules provided herein include a 5′ UTR comprising viral and non-viral sequences. Accordingly, a 5′ UTR included in nucleic acid molecules provided herein can comprise a combination of viral and non-viral 5′ UTR sequences. In some aspects, the 5′ UTR included in nucleic acid molecules provided herein is located upstream of or 5′ of the first polynucleotide that encodes one or more viral replication proteins. In other aspects, the 5′ UTR is located 5′ of or upstream of the first polynucleotide of nucleic acid molecules provided herein that encodes one or more viral replication proteins, and the first polynucleotide is located 5′ of or upstream of the second polynucleotide of nucleic acid molecules provided herein.

In one aspect, the 5′ UTR of nucleic acid molecules provided herein comprises an alphavirus 5′ UTR. A 5′ UTR from any alphavirus can be included in nucleic acid molecules provided herein, including 5′ UTR sequences from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O’Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV). In another aspect, the 5′ UTR comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:5 or a sequence of SEQ ID NO:41, for example. In yet another aspect, the 5′ UTR comprises a sequence of SEQ ID NO:5 or SEQ ID NO:41.

In some embodiments, the 5′ UTR comprises a sequence selected from the 5′ UTRs of human IL-6, alanine aminotransferase 1, human apolipoprotein E, human fibrinogen alpha chain, human transthyretin, human haptoglobin, human alpha-1-antichymotrypsin, human antithrombin, human alpha-1-antitrypsin, human albumin, human beta globin, human complement C3, human complement C5, SynK (thylakoid potassium channel protein derived from the cyanobacteria, Synechocystis sp.), mouse beta globin, mouse albumin, and a tobacco etch virus, or fragments of any of the foregoing. Preferably, the 5′ UTR is derived from a tobacco etch virus (TEV). In one aspect, the 5′ UTR includes a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to sequence of SEQ ID NO:35 or SEQ ID NO:49. In another aspect, the 5′ UTR includes a sequence of SEQ OD NO:35 or SEQ ID NO:49.

An mRNA or any other RNA described herein can comprise any 5′ UTR sequence provided herein. For example, an RNA described herein can comprise a 5′ UTR sequence that is derived from a gene expressed by Arabidopsis thaliana. In some aspects, the 5′ UTR sequence of a gene expressed by Arabidopsis thaliana is AT1G58420. Examples of 5 UTRs and 3′ UTRs are described in PCT/US2018/035419, the contents of which are herein incorporated by reference. Exemplary 5′ UTR sequences include sequences of SEQ ID NOs: 189-218, as shown in Table 1.

TABLE 1

Exemplary 5′ UTR Sequences

| | | |
|---|---|---|
| Name | Sequence | Seq ID No.: |
| TEV | UCAACACAACAUAUACAAAACAAACGAAUCUCAAGCAAUCAAGCAUUCUACUUCUAUUGCAGCAAUUUAAAUCAUUUCUUUUAAAGCAAAAGCAAUUUUCUGAAAAUUUUCACCAUUUACGAACGAUAG | SEQ ID NO: 189 |
| AT1G58420 | AUUAUUACAUCAAAACAAAAAGCCGCCA | SEQ ID NO: 190 |
| ARC5-2 | CUUAAGGGGGCGCUGCCUACGGAGGUGGCAGCCAUCUCU UCUCGGCAUCAAGCUUACCAUGGUGCCCCAGGCCCUGCUC UUGGUCCCGCUGCUGGUGUUCCCCCUCUGCUUCGGCAAGUUCCCCAUCUACACCAUCCCCGACAAGCUGGGGCCGUGGAGCCCCAUCGACAUCCACCACCUGUCCUGCCCCAACAACCUCGUGGUCGAGGACGAGGGCUGCACCAACCUGAGCGGGUUCUCCUAC | SEQ ID NO:191 |
| HCV | UGAGUGUCGU ACAGCCUCCA GGCCCCCCCC UCCCGGGAGA GCCAUAGUGG UCUGCGGAACCGGUGAGUAC ACCGGAAUUG CCGGGAAGAC UGGGUCCUUU CUUGGAUAAA CCCACUCUAUGCCCGGCCAU UUGGGCGUGC CCCCGCAAGA CUGCUAGCCG AGUAGUGUUG GGUUGCG | SEQ ID NO: 192 |
| HUMAN ALBUMIN | AAUUAUUGGUUAAAGAAGUAUAUUAGUGCUAAUUUCCCU CCGUUUGUCCUAGCUUUUCUCUUCUGUCAACCCCACACGC CUUUGGCACA | SEQ ID NO: 193 |
| EMCV | CUCCCUCCCC CCCCCCUAAC GUUACUGGCC GAAGCCGCUU GGAAUAAGGC CGGUGUGCGU UUGUCUAUAU GUUAUUUUCC ACCAUAUUGC CGUCUUUUGG CAAUGUGAGG GCCCGGAAAC CUGGCCCUGU CUUCUUGACG AGCAUUCCUA GGGGUCUUUC CCCUCUCGCC AAAGGAAUGC AAGGUCUGUU GAAUGUCGUG AAGGAAGCAG UUCCUCUGGA AGCUUCUUGA AGACAAACAA CGUCUGUAGC GACCCUUUGC AGGCAGCGGA ACCCCCCACC UGGCGACAGG UGCCUCUGCG GCCAAAAGCC ACGUGUAUAA GAUACACCUG CAAAGGCGGC ACAACCCCAG UGCCACGUUG UGAGUUGGAU AGUUGUGGAA AGAGUCAAAU GGCUCUCCUC AAGCGUAUUC AACAAGGGGC UGAAGGAUGC CCAGAAGGUA CCCCAUUGUA UGGGAUCUGA UCUGGGGCCU CGGUGCACAU GCUUUACGUG UGUUUAGUCG AGGUUAAAAA ACGUCUAGGC CCCCCGAACC ACGGGGACGU GGUUUUCCUU UGAAAAACAC GAUGAUAAU | SEQ ID NO: 194 |
| AT1G67090 | CACAAAGAGUAAAGAAGAACA | SEQ ID NO: 195 |
| AT1G35720 | AACACUAAAAGUAGAAGAAAA | SEQ ID NO: 196 |
| AT5G45900 | CUCAGAAAGAUAAGAUCAGCC | SEQ ID NO:197 |
| AT5G61250 | AACCAAUCGAAAGAAACCAAA | SEQ ID NO:198 |
| AT5G46430 | CUCUAAUCACCAGGAGUAAAA | SEQ ID NO:199 |
| AT5G47110 | GAGAGAGAUCUUAACAAAAAA | SEQ ID NO:200 |
| AT1G03110 | UGUGUAACAACAACAACAACA | SEQ ID NO:201 |
| AT3G12380 | CCGCAGUAGGAAGAGAAAGCC | SEQ ID NO:202 |
| AT5G45910 | AAAAAAAAAAGAAAUCAUAAA | SEQ ID NO:203 |
| AT1G07260 | GAGAGAAGAAAGAAGAAGACG | SEQ ID NO:204 |
| AT3G55500 | CAAUUAAAAAUACUUACCAAA | SEQ ID NO:205 |
| AT3G46230 | GCAAACAGAGUAAGCGAAACG | SEQ ID NO:206 |
| AT2G36170 | GCGAAGAAGACGAACGCAAAG | SEQ ID NO:207 |
| AT1G10660 | UUAGGACUGUAUUGACUGGCC | SEQ ID NO:208 |
| AT4G14340 | AUCAUCGGAAUUCGGAAAAAG | SEQ ID NO:209 |
| AT1G49310 | AAAACAAAAGUUAAAGCAGAC | SEQ ID NO:210 |
| AT4G14360 | UUUAUCUCAAAUAAGAAGGCA | SEQ ID NO:211 |
| AT1G28520 | GGUGGGGAGGUGAGAUUUCUU | SEQ ID NO:212 |
| AT1G20160 | UGAUUAGGAAACUACAAAGCC | SEQ ID NO:213 |
| AT5G37370 | CAUUUUUCAAUUUCAUAAAAC | SEQ ID NO:214 |
| AT4G11320 | UUACUUUUAAGCCCAACAAAA | SEQ ID NO:215 |
| AT5G40850 | GGCGUGUGUGUGUGUUGUUGA | SEQ ID NO:216 |
| AT1G06150 | GUGGUGAAGGGGAAGGUUUAG | SEQ ID NO:217 |
| AT2G26080 | UUGUUUUUUUUUGGUUUGGUU | SEQ ID NO:218 |

Additional exemplary 5′ UTR sequences of SEQ ID Ns:233-279 are shown in Table 2.

TABLE 2

Exemplary 5’ UTR Sequences

| SEQ ID NO. | SEQUENCE | SOURCE/NAME |
|---|---|---|
| 233 | AACUUAAAAAAAAAAAUCAAA | SYNECHOCYSTIS sp. PCC6803 POTASSIUM CHANNEL (SynK) |
| 234 | UCAAGCUUUUGGACCCUCGUACAGAAGCUAAUACGACUCACUAUAGGGAAAUAAGAGAGAAAAGAAGAGUAAGAAGAAAUAUAAGAGCCACC | SYNTHETIC SEQUENCE |
| 235 | CACAUUUGCUUCUGACAUAGUUGUGUUGACUCACAACCCCAGAAACAGACAUC | MOUSE BETA GLOBIN |
| 236 | ACAUUUGCUUCUGACACAACUGUGUUCACU AGCAACCUCAAACAGACACC | HUMAN BETA GLOBIN |
| 237 | UGCACACAGAUCACCUUUCCUAUCAACCCC ACUAGCCUCUGGCAAA | MOUSE ALBUMIN |
| 238 | CAUAAACCCUGGCGCGCUCGCGGGCCGGCACUCUUCUGGUCCCCACAGACUCAGAGAGAACCCACC | HUMAN ALPHA GLOBIN |
| 239 | AUAAAAAGACCAGCAGAUGCCCCACAGCAC UGCUCUUCCAGAGGCAAGACCAACCAAG | HUMAN HAPTOGLOBIN |
| 240 | AGACAAGGUUCAUAUUUGUAUGGGUUACUUAUUCUCUCUUUGUUGACUAAGUCAAUAAUCAGAAUCAGCAGGUUUGCAGUCAGAUUGGCAGGGAUAAGCAGCCUAGCUCAGGAGAAGUGAGUAUAAAAGCCCCAGGCUGGGAGCAGCCAUCACAGAAGUCCACUCAUUCUUGGCAGG | HUMAN TRANSTHYRETIN |
| 241 | AGAUAAAAAGCCAGCUCCAGCAGGCGCUGCUCACUCCUCCCCAUCCUCUCCCUCUGUCCCUCUGUCCCUCUGACCCUGCACUGUCCCAGCACC | HUMAN COMPLEMENT C3 |
| 242 | UAUAUCCGUGGUUUCCUGCUACCUCCAACC | HUMAN COMPLEMENT C5 |
| 243 | GGCACCACCACUGACCUGGGACAGUGAAUCGACA | HUMAN ALPHA-1-ANTITRYPSIN |
| 244 | AUUCAUGAAAAUCCACUACUCCAGACAGAC GGCUUUGGAAUCCACCAGCUACAUCCAGCUCCCUGAGGCAGAGUUGAGA | HUMAN ALPHA-1-ANTICHYMOTRYPSIN |
| 245 | AAUAUUAGAGUCUCAACCCCCAAUAAAUAU AGGACUGGAGAUGUCUGAGGCUCAUUCUGCCCUCGAGCCCACCGGGAACGAAAGAGAAGCUCUAUCUCCCCUCCAGGAGCCCAGCU | HUMAN INTERLEUKIN 6 |
| 246 | AGGAUGGGAACUAGGAGUGGCAGCAAUCCUUUCUUUCAGCUGGAGUGCUCCUCAGGAGCCAGCCCCACCCUUAGAAAAG | HUMAN FIBRINOGEN ALPHA CHAIN |
| 247 | AGGGGGAGCCCUAUAAUUGGACAAGUCUGGGAUCCUUGAGUCCUACUCAGCCCCAGCGGAGGUGAAGGACGUCCUUCCCCAGGAGCCGACUGGCCAAUCACAGGCAGGAAG | HUMAN APOLIPOPROTEIN E |
| 248 | AGACGGGUGGGGCGGGGCCCAACUGUCCCCAGCUCCUUCAGCCCUUUCUGUCCCUCCCAGUGAGGCCAGCUGCGGUGAAGAGGGUGCUCUCUUGCCUGGAGUUCCCUCUGCUACGGCUGCCCCCUCCCAGCCCUGGCCCACUAAGCCAGACCCAGCUGUCGCCAUUCCCACUUCUGGUCCUGCCACCUCCUGAGCUGCCUUCCCGCCUGGUCUGGGUAGAGUC | ALANINE AMINOTRANSFERASE 1 |
| 249 | CAGAUCGCCUGGAGACGCCAUCCACGCUGUUUUGACCUCCAUAGAAGACACCGGGACCGAUCCAGCCUCCGCGGCCGGGAACGGUGCAUUGGAACGCGGAUUCCCCGUGCCAAGAGUGACUCACCGUCCUUGACACG | HHV |
| 250 | GGGAGAAAGCUUACCAUGGUGCCCCAGGCCCUGCUCUUGGUCCCGCUGCUGGUUUCCCCCUCUGCUUCGGCAAGUUCCCCAUCUACACCAUCCCCGACAAGCUGGGGCCGUGGAGCCCCAUCGACAUCCACCACCUGUCCUGCCCCAACAACCUCGUGGUCGAGGACGAGGGCUGCACCAACCUGAGCGGGUUCUCCUAC | ARC5-1 |
| 251 | GGGGCGCUGCCUACGGAGGUGGCAGCCAUCUCCUUCUCGGCAUCAAGCUUACCAUGGUGCCCCAGGCCCUGCUCUUGGUCCCGCUGCUGGUGUUCCCCCUCUGCUUCGGCAAGUUCCCCAUCUACACCAUCCCCGACAAGCUGGGGCCGUGGAGCCCCAUCGACAUCCACCACCUGUCCUGCCCCAACAACCUCGUGGUCGAGGACGAGGGCUGCACCAACCUGAGCGGGUUCUCCUAC | ARC5-2 |
| 252 | GAAUAAAUGUAUAGGGGGAAAGGCAGGAGCCUUGGGGUCGAGGAAAACAGGUAGGGUAUAAAAAGGGCACGCAAGGGACCAAGUCCAGCAUCCUAGAGUCCAGAUUCCAAACUGCUCAGAGUCCUGUGGACAGAUCACUGCUUGGCA | Mouse GROWTH HORMONE |
| 253 | GACACUUCUGAUUCUGACAGACUCAGGAAGAAACC | MOUSE HEMOGLOBIN ALPHA |
| 254 | UGCAAACACAGAAAUGGAGGAGGAGGGGAAGGAGGAGGAGGAGGAGAAGGAGGAGGAGGUGGUGGUGGUGGUGGUGGGAUAAAACCCCUGAGGCAUAAAGGGCUCGGCCGGAGUCAGCACAGCCCAGCCCUUCCAGAGAGAGGCAAGAGAGGUCCACG | MOUSE HAPTOGLOBIN |
| 255 | CUAAUCUCCCUAGGCAAGGUUCAUAUUUGUGUAGGUUACUUAUUCUCCUUUUGUUGACUAAGUCAAUAAUCAGAAUCAGCAGGUUUGGAGUCAGCUUGGCAGGGAUCAGCAGCCUGGGUUGGAAGGAGGGGGUAUAAAAGCCCCUUCACCAGGAGAAGCCGUCACACAGAUCCACAAGCUCCUGACAGG | MOUSE TRANSTHYRETIN |
| 256 | AUAGGUAAUUUUAGAAAUAGAUCUGAUUU GUAUCUGAGACAUUUUAGUGAAGUGGUGAGAUAUAAGACAUAAUCAGAAGACAUAUCUACCUGAAGACUUUAAGGGGAGAGCUCCCUCCCCCACCUGGCCUCUGGACCUCUCAGAUUUAGGGGAAAGAACCAGUUUUCGGAGUGAUCGUCUCAGUCAGCACCAUCUCUGUAGGAGCAUCGGCC | MOUSE ANTITHROMBIN |
| 257 | AGAGAGGAGAGCCAUAUAAAGAGCCAGCGGCUACAGCCCCAGCUCGCCUCUGCCCACCCCUGCCCCUUACCCCUUCAUUCCUUCCACCUU UUUCCUUCACU | MOUSE COMPLEMENT C3 |
| 258 | UUUAAAAGGAAAGUGGUUACAGGGAGGCCAUGCCCAUGGGUUU | MOUSE COMPLEMENT C5 |
| 259 | AGUCCUUAGACUGCACAGCAGAACAGAAGG CAUG | MOUSE HEPCIDIN |
| 260 | CCCCCAUAUCCCCCUUGGCUCCCAUUGCUUAAAUACAGACUAGGACAGGGCUCUGUCUCCUCAGCCUCGGUCACCACCCAGCUCUGGGACAGCAAGCUGAAA | MOUSE ALPHA-1-ANTITRYPSIN |
| 261 | AGUCAGUCCUCCUUCGCUUCAGCUCCAGUUCUCCUCAUGAGCCAUCCCUAAACGCAGACACC | MOUSE FIBRINOGEN ALPHA CHAIN |
| 262 | UUUCCUCUGCCCUGCUGUGAAGGGGGAGAGAACAACCCGCCUCGUGACAGGGGGCUGGCACAGCCCGCCCUAGCCCUGAGGAGGGGGCGGGACAGGGGGAGUCCUAUAAUUGGACCGGUCUGGGAUCCGAUCCCCUGCUCAGACCCUGGAGGCUAAGGACUUGUUUCGGAAGGAGCUGACUGGCCAAUCACAAUUGCGAAG | APOLIPOPROTEIN E |
| 263 | GGCCGGCCACCGGGUUUGGGAGCAGCCCAGGCUCACCUUAACCGGAGCGGUGCGGACGGUCCCGCGGCGACAGGGCUAAUCUCGGCAGGUUCGCG | ALANINE AMINOTRANSFERASE |
| 264 | GUCCUGGACUGACUCCCACAACUCUGCCAGUCUCCAGCCCCUGCCCUUCAGUGGUACAG | CYTOCHROME P450, FAMILY 1(CYP1A2) |
| 265 | UUUAAGUCAACACCAGGAACUAGGACACAG UUGUCCAGGUGCUGUUGGCCAGUCCCAAC | PLASMINOGEN |
| 266 | AAGGAGCUGGGGAGUGGAGUGUAGGCACUAUAACCUGAAAGACGUGGUCCUGACAGGAGGACAAUUCUAUUCCCUACCAAA | MOUSE MAJOR URINARY PROTEIN 3 (MUP3) |
| 267 | ACCAGCCAGAAGCCACAGUCUCAUC | MOUSE FVII |
| 268 | AAACAGAGCAGGCAGGGGCCCUGAUUCACUGGCCGCUGGGGCCAGGGUUGGGGGCUGGGGGUGCCCACAGAGCUUGACUAGUGGGAUUUGGGGGGGCAGUGGGUGCAGCGAGCCCGGUCCGUUGACUGCCAGCCUGCCGGCAGGUAGACACCGGCCGUGGGUGGGGGAGGCGGCUAGCUCAGUGGCCUUGGGCCGCGUGGCCUGGUGGCAGCGGAGCC | HNF-1ALPHA |
| 269 | GGACUUCAGCAGGACUGCUCGAAACAUCCCACUUCCAGCACUGCCUGCGGUGAAGGAACCAGCAGCC | MOUSE ALPHA-FETOPROTEIN |
| 270 | AGGGCCUCGUGGGGGGCGGGAAGGUACUGUCCCAUAUAAGCCUCUGCUCUUGGGGCUCAACCGCUCGCACCCGCUGCGCUGCACAGGGGGAGAAAAGGAGCCCAGGGUGUGAGCCGGACAACUUCUGGUCCUCUCCUUCCAUCUCCUUACCGGCGUCCCCACCUCAGGACUUUUCCCGCAGGCUGCGAGGGGACCCACAGUUCGUGGCCACUUGCCUCCUGGGGAGGGCGACUCUCCUCCCAUCCACUCAAG | MOUSE FIBRONECTIN |
| 271 | GGGGGAAAAAAAAACAGCCAAAAUAUGCCA AAAAGCUUCUCACAACAGCUCCUCAGUAGA AGCAGGGGCCACUUGGGAAAGCCAGGGCCUGGACGCUAAUGUUCCAGGCUACAUCAUAGGUCCCUUUUCGCUCAGUGAGGCCACCAUCACCACACCAUGGCCACGUAGGCCUCCAGCCAGGGCAACAGGACCUGGAGGCCACCCAAGACUGCAGCUGGCUGCCGCUGGGUCCCCGGGCCAGCUCUUGGCCCCG | MOUSE RETINOL BINDING PROTEIN 4, PLASMA (RBP4) |
| 272 | GAACCGCGGCGAGGAGGGGGGUCGGAGGCCCAGACUUAUAAAGGCUGCUGGACCCGCGCUACCCGCCAGACCCCGCCGCCCGGAUCCCCCGCGCUGCCUGUCGCCCCACGUGACCACACUACUAAGCUUGGUCGCC | MOUSE PHOSPHOLIPID TRANSFER PROTEIN (PLTP) |
| 273 | AGGGACUCAUCAACCAGGCCUGGCCUCUGAGUUCAACGCAGAGCUAGCUGGGAAAUGUUCCGGAUGUUGGCCAAGGCCAGUGUGACGCUGGGCUCCAGAGCGGCAGGUUGGGUCCGGACC | MOUSE ALANINE-GLYOXYLATE AMINOTRANSFERASE (AGXT) |
| 274 | GCUGCCCCUGUGCUGACUGCUGACAGCUGACUGACGCUCGCAGCUAGCAGGUACUUCUGGGUUGCUAGCCCAGAGCCCUGGGCCGGUGACCCUGUUUUCCCUACUUCCCGUCUUUGACCUUGGGUGCCUUCCAACCUUCUGUUGCC | ALDEHYDE DEHYDROGENASE 1 FAMILY, MEMBER L1 (ALDH1L1) |
| 275 | GGGUGCUAAAAGAAUCACUAGGGUGGGGAGGCGGUCCCAGUGGGGCGGGUAGGGGUGUGUGCCAGGUGGUACCGGGUAUUGGCUGGAGGAAGGGCAGCCCGGGGUUCGGGGCGGUCCCUGAAUCUAAAGGCCCUCGGCUAGUCUGAUCCUUGCCCUAAGCAUAGUCCCGUUAGCCAACCCCCUACCCGCCGUGGGCUCUGCUGCCCGGUGCUCGUCAGC | FUMARYLACETOACETATE HYDROLASE (FAH) |
| 276 | AGGAGGACCUUGGCCAGCGGGCAGAAUGGCAGUUGGUAGAGGAAGGGAGCAAGGGGGUGUUUCCUGGGACAGGGGGGCGGAGACCUGGAGACUAUAGGCUCCCCCAGGACUCAAGUUCAUUGAGUUUCUGCAGACACUGAACGGCUUUCAGUCUUCCCGCUGUGACUAUCACCUGUGGGCUCCACCUGCCUGCACCUUUAGUCAGCACCUUUAGCCAGCACCUGCGCCAGACCCCAGCA | FRUCTOSE BISPHOSPHATASE 1 (FBP1) |
| 277 | AGGCGCCGGUCAGG | MOUSE GLYCINE N-METHYLTRANSFERASE (GNMT) |
| 278 | ACCAUCAACC | MOUSE 4-HYDROXYPHENYLPYRUVIC ACID DIOXYGENASE (HPD) |
| 279 | UCUGCCCCACCCUGUCCUCUGGAACCUCUGCGAGAUUUAGAGGAAAGAACCAGUUUUCAGGCGGAUUGCCUCAGAUCACACUAUCUCCACUUGCCCAGCCCUGUGGAAGAUUAGCGGCC | HUMAN ANTITHROMBIN |

### 3′ Untranslated Region (3′ UTR)

In some aspects, nucleic acid molecules provided herein further include a 3′ untranslated region (3′ UTR). Any 3′ UTR sequence can be included in nucleic acid molecules provided herein. In one aspect, nucleic acid molecules provided herein include a viral 3′ UTR. In another aspect, nucleic acid molecules provided herein include a non-viral 3′ UTR. Any non-viral 3′ UTR can be included in nucleic acid molecules provided herein, such as 3′ UTRs of transcripts expressed in any cell or organ, including muscle, skin, subcutaneous tissue, liver, spleen, lymph nodes, antigen-presenting cells, and others. In some aspects, nucleic acid molecules provided herein include a 3′ UTR comprising viral and non-viral sequences. Accordingly, a 3′ UTR included in nucleic acid molecules provided herein can comprise a combination of viral and non-viral 3′ UTR sequences. In one aspect, the 3′ UTR is located 3′ of or downstream of the second polynucleotide of nucleic acid molecules provided herein that comprises a first transgene encoding a first antigenic protein or a fragment thereof. In another aspect, the 3′ UTR is located 3′ of or downstream of the second polynucleotide of nucleic acid molecules provided herein that comprises a first transgene encoding a first antigenic protein or a fragment thereof, and the second polynucleotide is located 3′ of or downstream of the first polynucleotide of nucleic acid molecules provided herein.

In one aspect, the 3′ UTR of nucleic acid molecules provided herein comprises an alphavirus 3′ UTR. A 3′ UTR from any alphavirus can be included in nucleic acid molecules provided herein, including 3′ UTR sequences from Venezuelan Equine Encephalitis Virus (VEEV), Eastern Equine Encephalitis Virus (EEEV), Everglades Virus (EVEV), Mucambo Virus (MUCV), Semliki Forest Virus (SFV), Pixuna Virus (PIXV), Middleburg Virus (MIDV), Chikungunya Virus (CHIKV), O’Nyong-Nyong Virus (ONNV), Ross River Virus (RRV), Barmah Forest Virus (BFV), Getah Virus (GETV), Sagiyama Virus (SAGV), Bebaru Virus (BEBV), Mayaro Virus (MAYV), Una Virus (UNAV), Sindbis Virus (SINV), Aura Virus (AURAV), Whataroa Virus (WHAV), Babanki Virus (BABV), Kyzylagach Virus (KYZV), Western Equine Encephalitis Virus (WEEV), Highland J Virus (HJV), Fort Morgan Virus (FMV), Ndumu Virus (NDUV), Salmonid Alphavirus (SAV), or Buggy Creek Virus (BCRV). In another aspect, the 3′ UTR comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:9 or a sequence of SEQ ID NO:45, for example. In yet another aspect, the 3′ UTR further comprises a poly-A sequence. In a further aspect, the 3′ UTR comprises a sequence of SEQ ID NO:9 or SEQ ID NO:45. In yet a further aspect, the 3′ UTR comprises a sequence of SEQ ID NO:8 or a sequence of SEQ ID NO:44, for example.

In some embodiments, the 3′ UTR comprises a sequence selected from the 3′ UTRs of alanine aminotransferase 1, human apolipoprotein E, human fibrinogen alpha chain, human haptoglobin, human antithrombin, human alpha globin, human beta globin, human complement C3, human growth factor, human hepcidin, MALAT-1, mouse beta globin, mouse albumin, and Xenopus beta globin, or fragments of any of the foregoing. In some embodiments, the 3′ UTR is derived from Xenopus beta globin. Any 3′ UTR provided herein can include a poly-A tail, as detailed further below. In some embodiments, the 3′ UTR includes a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:36, SEQ ID NO:37, SEQ ID NO:50, or SEQ ID NO:51. In some embodiments, the 3′ UTR includes a sequence of SEQ ID NO:36, SEQ ID NO:37, SEQ ID NO:50, or SEQ ID NO:51. A 3′ UTR provided herein can be included in any RNA molecule provided herein, including self-replicating RNA and mRNA molecules. Exemplary 3′ UTR sequences include SEQ ID NOs:219-225, as shown in Table 3.

TABLE 3

3′ UTR sequences

| | | |
|---|---|---|
| Name | Sequence | Seq ID No.: |
| XBG | CUAGUGACUGACUAGGAUCUGGUUACCACUAAACCAGCCUCAAGAACACCCGAAUGGAGUCUCUAAGCUACAUAAUACCAACUUACACUUACAAAAUGUUGUCCCCCAAAAUGUAGCCAUUCGUAUCUGCUCCUAAUAAAAAGAAAGUUUCUUCACAU | SEQ ID NO: 219 |
| HUMAN HAPTOGLOBIN | UGCAAGGCUGGCCGGAAGCCCUUGCCUGAAAGCAAGAUUUCAGCCUGGAAGAGGGCAAAGUGGACGGGAGUGGACAGGAGUGGAUGCGAUAAGAUGUGGUUUGAAGCUGAUGGGUGCCAGCCCUGCAUUGCUGAGUCAAUCAAUAAAGAGCUUUCUUUUGACCCAU | SEQ ID NO: 220 |
| HUMAN APOLIPOPROTEIN E | ACGCCGAAGCCUGCAGCCAUGCGACCCCACGCCACCCCGUGCCUCCUGCCUCCGCGCAGCCUGCAGCGGGAGACCCUGUCCCCGCCCCAGCCGUCCUCCUGGGGUGGACCCUAGUUUAAUAAAGAUUCACCAAGUUUCACGCA | SEQ ID NO: 221 |
| HCV | UAGAGCGGCAAACCCUAGCUACACUCCAUAGCUAGUUUCUUUUUUUUUUGUUUUUUUUUUUUUUUUUUUUUUUUUUUUUUUUUUUUUUUCCUUUCUUUUCCUUCUUUUUUUCCUCUUUUCUUGGUGGCUCCAUCUUAGCCCUAGUCACGGCUAGCUGUGAAAGGUCCGUGAGCCGCAUGACUGCAGAGAGUGCCGUAACUGGUCUCUCUGCAGAUCAUGU | SEQ ID NO: 222 |
| MOUSE ALBUMIN | ACACAUCACAACCACAACCUUCUCAGGCUACCCUGAGAAAAAAAGACAUGAAGACUCAGGACUCAUCUUUUCUGUUGGUGUAAAAUCAACACCCUAAGGAACACAAAUUUCUUUAAACAUUUGACUUCUUGUCUCUGUGCUGCAAUUAAUAAAAAAUGGAAAGAAUCUAC | SEQ ID NO: 223 |
| HUMAN ALPHA GLOBIN | GCUGGAGCCUCGGUAGCCGUUCCUCCUGCCCGCUGGGCCUCCCAACGGGCCCUCCUCCCCUCCUUGCACCGGCCCUUCCUGGUCUUUGAAUAAAGUCUGAGUGGGCAGCA | SEQ ID NO: 224 |
| EMCV | UAGUGCAGUCAC UGGCACAACG CGUUGCCCGG UAAGCCAAUC GGGUAUACAC GGUCGUCAUACUGCAGACAG GGUUCUUCUA CUUUGCAAGA UAGUCUAGAG UAGUAAAAUA AAUAGUAUAAG | SEQ ID NO: 225 |

Additional exemplary 3′ UTR sequences of SEQ ID NOs:280-317 are shown in Table 4.

TABLE 4

Exemplary 3′ UTR Sequences

| SEQ ID NO. | SEQUENCE | SOURCE/NAME |
|---|---|---|
| 280 | ACCCCCUUUCCUGCUCUUGCCUGUGAACAAU GGUUAAUUGUUCCCAAGAGAGCAUCUGUCAG UUGUUGGCAAAAUGAUAAAGACAUUUGAAAAUCUGUCUUCUGACAAAUAAAAAGCAUUUAUUUCACUGCAAUGAUGUUUU | MOUSE BETA GLOBIN |
| 281 | GCUCGCUUUCUUGCUGUCCAAUUUCUAUUAA AGGUUCCUUUGUUCCCUAAGUCCAACUACUA AACUGGGGGAUAUUAUGAAGGGCCUUGAGCA UCUGGAUUCUGCCUAAUAAAAAACAUUUAUUUUCAUUGCAA | HUMAN BETA GLOBIN |
| 282 | UGGCAUCCCUGUGACCCCUCCCCAGUGCCUC UCCUGGCCCUGGAAGUUGCCACUCCAGUGCC CACCAGCCUUGUCCUAAUAAAAUUAAGUUGC AUCAUUUUGUCUG | HUMAN GROWTH FACTOR |
| 283 | AAUGUUCUUAUUCUUUGCACCUCUUCCUAUU UUUGGUUUGUGAACAGAAGUAAAAAUAAAUACAAACUACUUCCAUCUCA | HUMAN ANTITHROMBIN |
| 284 | CCACACCCCCAUUCCCCCACUCCAGAUAAAG CUUCAGUUAUAUCUCACGUGUCUGGAGUUCU UUGCCAAGAGGGAGAGGCUGAAAUCCCCAGC CGCCUCACCUGCAGCUCAGCUCCAUCCUACU UGAAACCUCACCUGUUCCCACCGCAUUUUCU CCUGGCGUUCGCCUGCUAGUGUG | HUMAN COMPLEMENT C3 |
| 285 | AACCUACCUGCCCUGCCCCCGUCCCCUCCCUUCCUUAUUUAUUCCUGCUGCCCCAGAACAUAGGUCUUGGAAUAAAAUGGCUGGUUCUUUUGU UUUCCAAA | HUMAN HEPCIDIN |
| 286 | ACUAAGUUAAAUAUUUCUGCACAGUGUUCCC AUGGCCCCUUGCAUUUCCUUCUUAACUCUCU GUUACACGUCAUUGAAACUACACUUUUUUGG UCUGUUUUUGUGCUAGACUGUAAGUUCCUUG GGGGCAGGGCCUUUGUCUGUCUCAUCUCUGUAUUCCCAAAUGCCUAACAGUACAGAGCCAUGACUCAAUAAAUACAUGUUAAAUGGAUGAAU GAAUUCCUCUGAAACUCU | HUMAN FIBRINOGEN ALPHA CHAIN |
| 287 | GCACCCCAGCUGGGGCCAGGCUGGGUCGCCCUGGACUGUGUGCUCAGGAGCCCUGGGAGGCUCUGGAGCCCACUGUACUUGCUCUUGAUGCCUGGCGGGGUGGGGUGGGGGGGGUGCUGGGCCCCUGCCUCUCUGCAGGUCCCUAAUAAAGCUGUGUGGCAGUCUGACUCC | ALANINE AMINOTRANSFERASE 1 |
| 288 | GAUUCGUCAGUAGGGUUGUAAAGGUUUUUCUUUUCCUGAGAAAACAACCUUUUGUUUUCUCAGGUUUUGCUUUUUGGCCUUUCCCUAGCUUUAAAAAAAAAAAAGCAAAA | MALAT |
| 289 | GGACUAGUUAUAAGACUGACUAGCCCGAUGG GCCUCCCAACGGGCCCUCCUCCCCUCCUUGCACCGAGAUUAAU | ARC3-1 |
| 290 | GGACUAGUGCAUCACAUUUAAAAGCAUCUCAGCCUACCAUGAGAAUAAGAGAAAGAAAAUGAAGAUCAAUAGCUUAUUCAUCUCUUUUUCUUUUUCGUUGGUGUAAAGCCAACACCCUGUCUAAAAAACAUAAAUUUCUUUAAUCAUUUUGCCUCUUUUCUCUGUGCUUCAAUUAAUAAAAAAUGGAAAGAACCUAGAUCU | ARC3-2 |
| 291 | CCACUCACCAGUGUCUCUGCUGCACUCUCCUGUGCCUCCCUGCCCCCUGGCAACUGCCACCCCUGCGCUUUGUCCUAAUAAAAUUAAGAUGCAUCAUAUCACCCG | MOUSE GROWTH HORMONE |
| 292 | GCUGCCUUCUGCGGGGCUUGCCUUCUGGCCAUGCCCUUCUUCUCUCCCUUGCACCUGUACCUCUUGGUCUUUGAAUAAAGCCUGAGUAGGAAGAAAAAAAAAAAA | MOUSE HEMOGLOBIN ALPHA |
| 293 | UUCAGGGCUCACUAGAAGGCUGCACAUGGCAGGGCAGGCUGGGAGCCAUGGAAGAGGGGGAAGUGGAAGGGUUGGGCUAUACUCUGAUGGGUUCUAGCCCUGCACUGCUCAGUCAACAAUAAAAAAAUGUGCUUUGGACCCAUAAAAAAAAAAAAAAAAAAAA | MOUSE HAPTOGLOBIN |
| 294 | GAGACUCAGCCCAGGAGGACCAGGAUCUUGCCAAAGCAGUAGCAUCCCAUUUGUACCAAAACAGUGUUCUUGCUCUAUAAACCGUGUUAGCAGCUCAGGAAGAUGCCGUGAAGCAUUCUUAUUAAACCACCUGCUAUUUCAUUCAAACUGUGUUUCUUUUUUAUUUCCUCAUUUUUCUCCCCUGCUCCUAAAACCCAAAAUCUUCUAAAGAAUUCUAGAAGGUAUGCGAUCAAACUUUUUAAAGAAAGAAAAUACUUUUUGACUCAUGGUUUAAAGGCAUCCUUUCCAUCUUGGGGAGGUCAUGGGUGCUCCUGGCAACUUGCUUGAGGAAGAUAGGUCAGAAAGCAGAGUGGACCAACCGUUCAAUGUUUUACAAGCAAAACAUACACUAAGCAUGGUCUGUAGCUAUUAAAAGCACACAAUCUGAAGGGCUGUAGAUGCACAGUAGUGUUUUCCCAGAGCAUGUUCAAAAGCCCUGGGUUCAAUCACAAUACUGAAAAGUAGGCCAAAAAACAUUCUGAAAAUGAAAUAUUUGGGUUUUUUUUUAUAACCUUUAGUGACUAAAUAAAGACAAAUCUAAGAGACUAAAAAAAAAAAAAAAAAA | MOUSE TRANSTHYRETIN |
| 295 | AAUAUUCUUAAUCUUUGCACCUUUUCCUACUUUGGUGUUUGUGAAUAGAAGUAAAAAUAAAUACGACUGCCACCUCACGAGAAUGGACUUUUCCACUUGAAGACGAGAGACUGGAGUACAGAUGCUACACCACUUUUGGGCAAGUGAAGGGGGAGCAGCCAGCCACGGUGGCACAAACCUAUAUCCUGGUGCUUUUGAAGGUAGAAGCAGGGCGGUCAGGAGUUAAGGCCAGUUGAGGCUGGGCUGCAGAGUGAAAGACCAUGUCUCAAGAUGGUCUUUCUCCUCCCCAAAGUAGAAAAGAAAACCAUAAAAACAAGAGGUAAAUAUAUUACUAUUUCAUCUUAGAGGAUAGCAGGCAUCUUGAAAGGGUAGAGGGACCUUAAAUUCUCAUUAUUGCCCCCAUACUACAAACUAAAAAACAAACCCGAAUCAAUCUCCCAUAAAGACAGAGAUUCAAAUAAGAGUAUUAAACGUUUUAUUUCUCAAACCACUCACAUGCAUAAUGUUCUUAUACACAGUGUCAAAAUAAAGAGAAAUGCAUUUUUAUACAAAAAAAAAAAA | MOUSE ANTITHROMBIN |
| 296 | CUACAGCCCAGCCCUCUAAUAAAGCUUCAUUGUAUUUCACCCAUC | MOUSE COMPLEMENT C3 |
| 297 | AAAGUUCUGCUGCACGAAGAUUCCUCCUGCGGCGGGGGGAUUGCUCCUCCUCUGGCUUGGAAACCUAGCCUAGAAUCAGAUACACUUUCUUUAGAGUAAAGCACAAGCUGAUGAGUUACGACUUUGUGAAAUGGAUAGCCUUGAGGGGAGGCGAAAACAGGUCCCCCAAGGCUAUCAGAUGUCAGUGCCAAUAGACUGAAACAAGUCUGUAAAGUUAGCAGUCAGGGGUGUUGGUUGGGGCCGGAAGAAGAGACCCACUGAAACUGUAGCCCCUUAUCAAAACAUAUCCUUGCUUGAAAGAAAAAUACCAAGGACAGAAAAUGCCAUAAAAUCUUGACUUUGCACUC | MOUSE COMPLEMENT C5 |
| 298 | CCUAGAGCCACAUCCUGACCUCUCUACACCCCUGCAGCCCCUCAACCCCAUUAUUUAUUCCUGCCCUCCCCACCAAUGACCUUGAAAUAAAGACGAUUUUAUUUUCAAAAAAAAAAAAAAAAAA | MOUSE HEPCIDIN |
| 299 | CCACCCUAAAAUGUCAUCCUUCCUUCUGAAUUGGGUUCCUUCCAUUAAACACAGGCUGGCCUGGCUCGUGCCUGAUGCUACAGCAAGUCCUUGACUCUGUGGGUUGUGUGUGUGUGUGUGUGUGUGUGUGUGUGUGUGUGUGUGUGUGUGUGUGUGUGUGUGUGUGUGUCUGUGUGUGUGUGUGUCUUUAUGCCCUGAGUUUGUUGUGGACUUGAGAUCAUAGUAUGUCUUGAUAUCUCCUCCAGCCAUGCAAAUAGGUUGUGGGUAGAGGACUGUGGCUGAGACCACAGACUCUGGUCCAAGAACCAUCUGCUCUAAAAAAAAUAAAUCUGUCAUCUCUGGAAAAUAAAGAGGACAUGCUCAAUGACUCAGGGUCCAGC | MOUSE ALPHA-1-ANTITRYPSIN |
| 300 | CUGAAGGGUUAGAAAGUGGGGGCUCUGUUU UCUUUGCUCGGUUAUCCGAGAAGAAAGACAAAACGGAAGAUGAAGGUGUCACGGAUCUUGUGAACUUUUUAAAACUUUCAAGGUGCUAUUCCAUUGUUCUUUGUACUGUAGCUAAAUGUAACUGAGAUGAGUUACUGCUUUGAAAAAAUAAAGUUUUACAUUUUUUCCACCCUUUAAAAAAAAAAAA | MOUSE FIBRINOGEN ALPHA CHAIN |
| 301 | GUAUCCUUCUCCUGUCCUGCAACAACAUCCAUAUCCAGCCAGGUGGCCCUGUCUCAAGCACCUCUCUGGCCCUCUGGUGGCCCUUGCUUAAUAAAGAUUCUCCGAGCACAUUCUGAGUCUCUGUGAGUGAUUC AAAAAAAA | APOLIPOPROTEIN E |
| 302 | GGACGC CUCAGGCACC GGAGCCAGAC CCUCCCAAGA CCACCCAGGC CUUCCUCAAG GACUCUGCCU CAGACCUCAG ACAGGCCACC AACGCUGUUC AUCUUCAUUU CCCCAAGGAG ACUUCUUUCU UUGUGCCUUG AUGUUUGAGA GUUCUUCGAG CAAACAGUGG UUUUGCAAUG UCUCACAGGC CCUGUUUUUG UUUUUGUUUU UGUUUUGUUU UGUUUUGUUC UUUUUUUAAA UGCAACCAAA GUAGAGUCAA CCUGCUCGGC AGAUGUACUU GGAUUCUCUG AAUCGCUAUU CUGUUUGGAG AGUUCCUUUG GGUCUUAAGC AGCCAGAGUA CAUGGAAAUG AGAUUAUGUC AGAUCUGGAG AAACAAGCAG GUGUUGGGAA AUAUGUGACU UGACAUGAUA AGGGCUGGGA AUCCAGAAAU CAAUAGUGAG AUCCAUGAAA UCAAACCCUG ACCAGUGUGA AAAUGUAGCC UUUUGGACAG UAAGCCUGCA AGUCUAGUGA GAACUCAGAG AAAGCUGACC AUUCUGGUCU GAAGAUAGGC AGCGCAUCAC AGGCAAGAAU AUCGAAGUCA GUAGUAGGAC AGGGGUCACA UCAGAUACCA GCUCAAAUUG CACUAGCUAU CUAGAACAGU UUUCUCCAGG UUUGCCUGAG CCUUGAUGCA UACCAUCGCC CUCUGCUGGU CGCAGCAGAG AUAAGCAAGG GCUGAAAAUG GAGGCAAUCC UUUCCCAAGG CCCUGAAAGU UGUUUUUCAU GGUUUCAAAC UGAAUUUGGC UCAUUUGUAA CUAACUGAUC ACGGUGCCUG GUUACACUGG CUGCCAAGAA GGAGCGCAUG CAAUCUGAUU CAGUGCUCUC UUCACAUCAG UUUCCUGCCU CCCUCCCUCA UCUGCGGACA GCAUCCUAUC UCAUCAGGCU UCCCUGUGUG UCACAAAGUA GCAGCCACCA AGCAAAUAUA UUCCUUGAAU UAGCACACCU GGGUGGGCCA UGUGCGCACC AAGGAAACAG GUGCUAUAGG GAGCGCCAGG CCAGGCUUGU CUCUUAACUG UCUCGUUCUU CAGUGAGAGU GGGAAAGCUG UCCGGAGCUC CCGCGCAGGA GCCUGGGUAC CCACGCAGCG AGUCAAGGGA GUUUUCGGAG CCAGAGAGAG AAAGAUGUGA AGGCUGUGGA GUAAGGCUGA AACCAGCCUC CUGCCCUAUA GUCCCACACU GCAGGGGGUG CGACUUUAAA ACAGAACUUC AAGUUGUUAA CACUCACAAG CAUUGCAUUA CUGUGAAGGA AGUAGCCGCA UCCAUAACAG GAUGUGAUGG UCUACAGCUU UUCCUUUAAA AGCUGAAAAG GUACCAUGUG UGCUCGCUAG GCAUAUAAUC CAGAUAUGCU CCAGAGUUCU GAGAUUCUUC CAUGAAAGGU UAACUAGAAG CUAGAAUAUU UUUUUAUAUU UUUGUAACAA UUGGCUUUUU UCAUGGGGGG AGGGGAGUAG AGGGUUAGUA UUUAUAGUCC UAACAAGUCC AAAAAUUUUU AUAAGUGUCU UCAGAUUAUA AAUAACCCUC CAAAUUUUGC AAUGUUUACA UGUUUUUUUU UUAAGAUGAC AAAUAUGCUU GAUUUGCUUU UUAAAUAAAA GUUUAGCUGU UCUAAGAGAU UAACUUCAAG UAGGAUGGCU GGUUAUGAUA GUUUGGAUUU UCUACAGGUU CUGUUGCCAU GCCUUUUGGG UUUCAGCAUC ACUCGAGUCG CAGCAUGUGG GUGGGGCUGU GGAAACCUGG CCAGGCUGGA CCUGGUCAGC CACACCUCAG AGACAUUGUU UCCAUUUGGA UGUGAGCAGG CGCAGGCCUG CAUGCUCUUU CCUACUUAGC AUCAUCAGUU CUUCCGCCUC CUUAGCAUGG UUCUUUGUAA CAGCCAUGCU GGGAAGCUCU GAACAAUAAA AUACUUCCAG AGUGGU | ALANINE AMINOTRANSFERASE |
| 303 | AGAUUGUCGAGGCAUCGGUGGGGCCGUCACCCUUGUUUCUUUUCCUUUUUUAAAAAAAAAAAAAAAACAGCUUUUUUUUUUUUGAGAGAUAC AAUUCUUUCCCCAUUUAAUUCAUCUCCAAGC AAUUUUACAAUAGUGUCUAUCAUGUUCACCCCAUAACCCAUACUCAUUAGGACUUAUGAUUUAAGAUUCCUCCUACCCUGUCUUGCUUGCCGCACCUCAUGCUAAUCUAGUUUUUGACUCAAUAGAUUUGCCUACUCUGGCUGUCUCAUAUAAAUCGAAUGAAUUAUG | CYTOCHROME P450, FAMILY 1(CYP1A2) |
| 304 | CUAGGUGGAAGGCCGAGCAAAACCUCUGCUUACUAAAGCUUACUGAAUAUGGGGAGAGGGCUUAGGGUGUUUGGAAAAACUGACAGUAAUCAAACUGGGACACUACACUGAACCACAGCUUCCUGUCGCCCCUCAGCCCCUCCCCUUUUUUUGUAUUAUUGUGGGUAAAAUUUUCCUGUCUGUGGACUUCUGGAUUUUGUGACAAUAGACCAUCACUGCUGUGACCUUUGUUGAAAAUAAACUCGAUACUUACUUUG | PLASMINOGEN |
| 305 | AGAA UGGCCUGAGC CUCCAGUGUU GAGUGGAGAC UUUUCACCAG GACUCCAGCA UCAUCCCUUC CUAUCCAUAC AGACUCCCAU GCCAAGGUCU GUGAUCUGCU CUCCACCUGU CUCACAGAGA AGUGCAAUCC CGUUCUCUCC AGCAUGUUAC CUAGGAUAAC UCAUCAAGAA UCAAAGACUU UCUUUAAAUU UCUCUUUGCC AACACAUGGA AAUUCUCCAU UGAUUUCUUU CCUGUCCUGU UCAAUAAAUG AUUACACUUG CACUUAAAAA AAAAAAAA | MOUSE MAJOR URINARY PROTEIN 3 (MUP3) |
| 306 | CUCC UUGGAUAGCC CAACCCGUCC CAAGAAGGAA GCUACGGCCU GUGAAGCUGU UCUAUGGACU UUCCUGCUAU UCUUGUGUAA GGGAAGAGAA UGAGAUAAAG AGAGAGUGAA GAAAGCAGAG GGGGAGGUAA AUGAGAGAGG CUGGGAAAGG GGAAACAGAA AGCAGGGCCG GGGGAAGAGU CUAAGUUAGA GACUCACAAA GAAACUCAAG AGGGGCUGGG CAGUGCAGUC ACAGUCAGGC AGCUGAGGGG CAGGGUGUCC CUGAGGGAGG CGAGGCUCAG GCCUUGCUCC CGUCUCCCCG UAGCUGCCUC CUGUCUGCAU GCAUUCGGUC UGCAGUACUA CACAGUAGGU AUGCACAUGA GCACGUAGGA CACGUGAAUG UGCCGCAUGC AUGUGCGUGC CUGUGUGUCC AUCAUUGGCA CUGUUGCUCA CUUGUGCUUC CUGUGAGCAC CCUGUCUUGG UUUCAAUUAA AUGAGAAACA UGGUCAAAAA AAAAAAAAAA AAAAA | MOUSE FVII |
| 307 | CCGUG GUGACUGCCU CCCAGGAGCU GGGUCCCCAG GGCCUGCACU GCCUGCAUAG GGGGUGAGGA GGGCCGCAGC CACACUGCCU GGAGGAUAUC UGAGCCUGCC AUGCCACCUG ACACAGGCUG CUGGCCUUCC CAGAAGUCUA CGCAUUCAUU GACACUGCUG CUCCUCCAUC AUCAGGAAGG GAUGGCUCUG AGGUGUCUCA GCCUGACAAG CGAGCCUCGA GGAGCUGGAG GACGGCCCAA UCUGGGCAGU AUUGUGGACC ACCAUCCCUG CUGUUUAGAA UAGGAAAUUU AAUGCUUGGG ACAGGAGUGG GGAAGCUCGU GGUGCCCGCA CCCCCCCAGU CAGAGCCUGC AGGCCUUCAA GGAUCUGUGC UGAGCUCUGA GGCCCUAGAU CAACACAGCU GCCUGCUGCC UCCUGCACCU CCCCAGGCCA UUCCACCCUG CACCAGAGAC CCACGUGCCU GUUUGAGGAU UACCCUCCCC ACCACGGGGA UUUCCUACCC AGCUGUUCUG CUAGGCUCGG GAGCUGAGGG GAAGCCACUC GGGGCUCUCC UAGGCUUUCC CCUACCAAGC CAUCCCUUCU CCCAGCCCCA GGACUGCACU UGCAGGCCAU CUGUUCCCUU GGAUGUGUCU UCUGAUGCCA GCCUGGCAAC UUGCAUCCAC UAGAAAGGCC AUUUCAGGGC UCGGGUUGUC AUCCCUGUUC CUUAGGACCU GCAACUCAUG CCAAGACCAC ACCAUGGACA AUCCACUCCU CUGCCUGUAG GCCCCUGACA ACUUCCUUCC UGCUAUGAGG GAGACCUGCA GAACUCAGAA GUCAAGGCCU GGGCAGUGUC UAGUGGAGAG GGUACCAAGA CCAGCAGAGA GAAGCCACCU AAGUGGCCUG GGGGCUAGCA GCCAUUCUGA GAAAUCCUGG GUCCCGAGCA GCCCAGGGAA ACACAGCACA CAUGACUGUC UCCUCGGGCC UACUGCAGGG AACCUGGCCU UCAGCCAGCU CCUUUGUCAU CCUGGACUGU AGCCUACGGC CAACCAUAAG UGAGCCUGUA UGUUUAUUUA ACUUUUAGUA AAGUCAGUAA AAAGCAAAAA AAAAAAAAAA AAA | HNF-1 ALPHA |
| 308 | ACAUC UCCAGAAGGA AGAGUGGACA AAAAAAUGUG UUGACUCUUU GGUGUGAGCC UUUUGGCUUA ACUGUAACUG CUAGUACUUU AACCACAUGG UGAAGAUGUC CAUGUGAGAU UUCUAUACCU UAGGAAUAAA AACUUUUCAA CUAUUUCUCU UCUCCUAGUC UGCUUUUUUU UUAUUAAAAA AUACUUUUUU CCAUUU | MOUSE ALPHA-FETOPROTEIN |
| 309 | UCUU UCCAGCCCCA CCCUACAAGU GUCUCUCUAC CAAGGUCAAU CCACACCCCA GUGAUGUUAG CAGACCCUCC AUCUUUGAGU GGUCCUUUCA CCCUUAAGCC UUUUGCUCUG GAGCCAUGUU CUCAGCUUCA GCACAAUUUA CAGCUUCUCC AAGCAUCGCC CCGUGGGAUG UUUUGAGACU UCUCUCCUCA AUGGUGACAG UUGGUCACCC UGUUCUGCUU CAGGGUUUCA GUACUGCUCA GUGUUGUUUA AGAGAAUCAA AAGUUCUUAU GGUUUGGUCU GGGAUCAAUA GGGAAACACA GGUAGCCAAC UAGGAGGAAA UGUACUGAAU GCUAGUACCC AAGACCUUGA GCAGGAAAGU CACCCAGACA CCUCUGCUUU CUUUUGCCAU CUGACCUGCA GCACUGUCAG GACAUGGCCU GUGGCUGUGU GUUCAAACAC CCCUCCCACA GGACUCACUU UGUCCCAACA AUUCAGAUUG CCUAGAAAUA CCUUUCUCUU ACCUGUUUGU UAUUUAUCAA UUUUUCCCAG UAUUUUUAUA CGGAAAAAAU UGUAUUGAAG ACACUUUGUA UGCAGUUGAU AAGAGGAAUU CAGUAUAAUU AUGGUUGGUG AUUAUUUUUA UAAGCACAUG CCAACGCUUU ACUACUGUGG AAAGACAAGU GUUUUAAUAA AAAGAUUUAC AUUCCAUGAU GUGGACGUCA UUUCUUUUUU UUUUUAACAU CAUGUGUUUG GAGAG | MOUSE FIBRONECTIN |
| 310 | CAACGUCUA GGAUGUGAAG UUUGAAGAUU UCUGAUUAGC UUUCAUCCGG UCUUCAUCUC UAUUUAUCUU AGAAGUUUAG UUUCCCCCAC CUCCCCUACC UUCUCUAGGU GGACAUUAAA CCAUCGUCCA AAGUACAUGA GAGUCACUGA CUCUGUUCAC ACAACUGUAU GUCUUACUGA AGGUCCCUGA AAGAUGUUUG AGGCUUGGGA UUCCAAACUU GGUUUAUUAA ACAUAUAGUC ACCAUCUUCC UAU | MOUSE RETINOL BINDING PROTEIN 4, PLASMA (RBP4) |
| 311 | GC CCAUCACCCC ACCUGGGUGG CUGGCAUUCA GGAACCUAAC UGAAGUCUUC UCUGCACCCC CUGCCAACCC CUUCCCAUCU ACAGUGUUAG UGGUCCCGGU GCCACAGAGA AGAGCCCAGU UGGAAGCUAU ACCCGAUUUA AUUCCAGAAU UAGUCAACCA UCAAUUAGAA UCCAUCCACC CCCCUC | MOUSE PHOSPHOLIPID TRANSFER PROTEIN (PLTP) |
| 312 | G CAUCCUCUCA CCAGACUAUG CCCUCCUGGA GGGGCUGGGA AUAUAGCAAG AACGAAAAGA CUGUGCAAGG CCUAGAGCCA GCAAAGAUGC UGAUGUAGCC AGGCCAUGCC GGAAGGAGCA GGGUGAAGCU UCCCCUCUCC CUACAAAUGG AACCUUGUGG AAACAGGAUG CUAAACACCU UCUGAUGGAG CUGUUGCCUG CAGGCCACUG GUCUUUGGGA AUUUUCAAUA AAGUGCUUGC GAGGAAUCUC CUA | MOUSE ALANINE-GLYOXYLATE AMINOTRANSFERASE (AGXT) |
| 313 | AGCCA AGACUGUGAU ACUUCUCCUG UACCCUGUUG ACCUCAGGGA GUGCUGACCC UGUCUGGUGA CUUAGCACCC UCCUGUCCCC AGCACUGCUC CUUUCAGCUG CUGGAGCUCU UGGCCUGGAC CCCUGCUGGU GACAGGACAC CCUCUGAACA AUCAGAAGUG GCUCCAAGUG GAGUGAGCAG UCAUGUCCCC CAUGAAUAAA AAUUGUGAGC AGAGGUCGCC UACAAAAAAA AAAAAAAA | ALDEHYDE DEHYDROGENASE 1 FAMILY, MEMBER L1 (ALDH1L1) |
| 314 | A GCUCCGGAAG UCACAAGACA CACCCUUGCC UUAUGAGGAU CAUGCUACCA CUGCAUCAGU CAGGAAUGAA UAAAGCUACU UUGAUUGUGG GAAAUGCCAC AGAAAAAAAA AAAAAAA | FUMARYLACETOACETATE HYDROLASE (FAH) |
| 315 | AGGC CAGCCUUGCC CCUGCCCCAG AGCAGAGCUC AAGUGACGCU ACUCCAUUCU GCAUGUUGUA CAUUCCUAGA AACAAACCUA ACAGCGUGGA UAGUUUCACA GCUUAAUGCU UUGCAAUGCC CAAGGUCACU UCAUCCUCAU GCUAUAAUGC CACUGUAUCA GGUAAUAUAU AUUUUGAGUA GGUGAAGGAG AAAUAAACAC AUCUUUCCUU UAUAAAUUA | FRUCTOSE BISPHOSPHATASE 1 (FBP1) |
| 316 | GUUU CUCCGGCUCC CAGAAGCCCA UGCUCAGGCA AUGGCCCCUA CCCUAAGACC AUCCCCUAAU GCAGAUAUUG CAUUUGGGUG CAGAUGUGGG GGUCGGGCAA ACGGAGUAAA CAAUACAGUC UGCAUUCUCC AAAAAAAAAA AA | MOUSE GLYCINE N-METHYLTRANSFERASE (GNMT) |
| 317 | GCCCCCAU CCACACAUGG ACCACGCAAA GUGCUGGACA CAUCAGUCAU CUCCAACUGG CUGAAAGGCU GAACCUCAGG GCUCCACCCA CGUCAUGGCC ACGCCCCCUC UAUUACAAGA GUCCGCCUUG CCUGAGUCCU CCCUGCUGAG UAAAGCUACC CUCCCAGGUC CAAAAAAAAA AAAAAAAAAA AAAAAAAAAA AAAAAAAAAA AAAAAAAA | MOUSE 4- HYDROXYPHENYLPYRUVIC ACID DIOXYGENASE (HPD) |

### Triple Stop Codon

In some embodiments, RNA molecules provided herein, including self-replicating RNA and mRNA, may comprise a sequence immediately downstream of a coding region (i.e., ORF) that creates a triple stop codon. A triple stop codon is a sequence of three consecutive stop codons. The triple stop codon can ensure total insulation of an expression cassette and may be incorporated to enhance the efficiency of translation. In some embodiments, RNA molecules of the disclosure may comprise a triple combination of any of the sequences UAG, UGA, or UAA immediately downstream of an ORF described herein. The triple combination can be three of the same codons, three different codons, or any other permutation of the three stop codons.

### Translation Enhancers and Kozak Sequences

For translation initiation, proper interactions between ribosomes and mRNAs must be established to determine the exact position of the translation initiation region. However, ribosomes also must dissociate from the translation initiation region to slide toward the downstream sequence during mRNA translation. Translation enhancers upstream from initiation sequences of mRNAs enhance the yields of protein biosynthesis. Several studies have investigated the effects of translation enhancers. In some embodiments, an RNA molecule described herein, such as a self-replicating RNA or an mRNA, comprises a translation enhancer sequence. These translation enhancer sequences enhance the translation efficiency of a self-replicating RNA or mRNA of the disclosure and thereby provide increased production of the protein encoded by the RNA. The translation enhancer region may be located in the 5′ or 3′ UTR of a self-replicating RNA or an mRNA sequence. Examples of translation enhancer regions include naturally-occurring enhancer regions from the TEV 5′ UTR and the Xenopus beta-globin 3′ UTR. Exemplary 5′ UTR enhancer sequences include but are not limited to those derived from mRNAs encoding human heat shock proteins (HSP) including HSP70-P2, HSP70-M1 HSP72-M2, HSP17.9 and HSP70-P1. Exemplary translation enhancer sequences used in accordance with the embodiments of the present disclosure are represented by SEQ ID NOs:226-230, as shown in Table 5.

TABLE 5

5′ UTR Enhancers

| | | |
|---|---|---|
| Name | Sequence | Seq ID No.: |
| HSP70-P2 | GUCAGCUUUCAAACUCUUUGUUUCUUGUUUGUUGAUUGAGAAUA | SEQ ID NO:226 |
| HSP70-M1 | CUCUCGCCUGAGAAAAAAAAUCCACGAACCAAUUUCUCAGCAACCAGCAGCACG | SEQ ID NO:227 |
| HSP72-M2 | ACCUGUGAGGGUUCGAAGGAAGUAGCAGUGUUUUUUGUUCCUAGAGGAAGAG | SEQ ID NO:228 |
| HSP17.9 | ACACAGAAACAUUCGCAAAAACAAAAUCCCAGUAUCAAAAUUCUUCUCUUUUUUUCAUAUUUCGCAAAGAC | SEQ ID NO:229 |
| HSP70-P1 | CAGAAAAAUUUGCUACAUUGUUUCACAAACUUCAAAUAUUAUUCAUUUAUUU | SEQ ID NO:230 |

In some embodiments, a self-replicating RNA or mRNA of the disclosure comprises a Kozak sequence. As is understood in the art, a Kozak sequence is a short consensus sequence centered around the translational initiation site of eukaryotic mRNAs that allows for efficient initiation of translation of the self-replicating RNA or mRNA. See, for example, Kozak, Marilyn (1988) Mol. and Cell Biol, 8:2737-2744; Kozak, Marilyn (1991) J. Biol. Chem, 266: 19867-19870; Kozak, Marilyn (1990) Proc Natl. Acad. Sci. USA, 87:8301-8305; and Kozak, Marilyn (1989) J. Cell Biol, 108:229-241. It ensures that a protein is correctly translated from the genetic message, mediating ribosome assembly and translation initiation. The ribosomal translation machinery recognizes the AUG initiation codon in the context of the Kozak sequence. A Kozak sequence may be inserted upstream of the coding sequence for the protein of interest, downstream of a 5′ UTR or inserted upstream of the coding sequence for the protein of interest and downstream of a 5′ UTR. In some embodiments, a self-replicating RNA or mRNA described herein comprises a Kozak sequence having the sequence GCCACC (SEQ ID NO: 231). A self-replicating RNA or mRNA described herein can comprise a partial Kozak sequence “p” having the nucleotide sequence GCCA (SEQ ID NO: 232).

### Transgenes

Transgenes included in nucleic acid molecules provided herein can encode an antigenic protein or a fragment thereof. In some embodiments, second polynucleotides of RNA molecules provided herein comprise a first transgene. A first transgene included in second polynucleotides of nucleic acid molecules provided herein can encode a first antigenic protein or a fragment thereof. A transgene included in second polynucleotides of RNA molecules provided herein can comprise a sequence encoding the full amino acid sequence of an antigenic protein or a sequence encoding any suitable portion or fragment of the full amino acid sequence of an antigenic protein. A transgene included in second polynucleotides of RNA molecules provided herein can also include a homolog of any antigenic protein provided herein. Any antigenic protein can be encoded by transgenes included in nucleic acid molecules provided herein. In one aspect, the first antigenic protein is a viral protein, a bacterial protein, a fungal protein, a protozoan protein, or a parasite protein. Transgenes included in RNA molecules provided herein can be expressed from a subgenomic RNA derived from a self-replicating RNA or from an mRNA.

In some aspects, the antigenic protein, when administered to a mammalian subject, raises an immune response to a pathogen, optionally wherein the pathogen is viral, bacterial, fungal, protozoan, or any other type of pathogen. In other aspects, the antigenic protein is expressed on the outer surface of the pathogen; while in further aspects, the antigen may be a non-surface antigen, e.g., useful as a T-cell epitope. The immune response may comprise an antibody response (usually including IgG) and/or a cell mediated immune response. The polypeptide immunogen will typically elicit an immune response that recognizes the corresponding pathogen polypeptide, but in some embodiments, the polypeptide may act as a mimotope to elicit an immune response that recognizes a saccharide. The immunogen can be a surface polypeptide, e.g., an adhesin, a hemagglutinin, an envelope glycoprotein, a spike glycoprotein, etc.

Any viral, bacterial, fungal, protozoan, parasite, or other protein can be encoded by transgenes included in RNA molecules provided herein. A protein from any infectious agent can be encoded by transgenes included in RNA molecules provided herein. As used herein, the term “infectious agent” refers to any agent capable of infecting an organism, including humans and animals, and causing disease or deterioration in health. The terms “infectious agent” and “infectious pathogen” may be used interchangeably, unless context clearly indicates otherwise.

In some aspects, the viral protein encoded by transgenes included in RNA molecules provided herein is a coronavirus protein, an orthomyxovirus protein, a paramyxovirus protein, a picornavirus protein, a flavivirus protein, a filovirus protein, a rhabdovirus protein, a togavirus protein, an arterivirus protein, a bunyavirus protein, an arenavirus protein, a reovirus protein, a bornavirus protein, a retrovirus protein, an adenovirus protein, a herpesvirus protein, a polyomavirus protein, a papillomavirus protein, a poxvirus protein, or a hepadnavirus protein. In other aspects, the antigenic protein is a SARS-CoV-2 protein, an influenza virus protein, a respiratory syncytial virus (RSV) protein, a human immunodeficiency virus (HIV) protein, a hepatitis C virus (HCV) protein, a cytomegalovirus (CMV) protein, a Lassa Fever Virus (LFV) protein, an Ebola Virus (EBOV) protein, a Mycobacterium protein, a Bacillus protein, a Yersinia protein, a Streptococcus protein, a Pseudomonas protein, a Shigella protein, a Campylobacter protein, a Salmonella protein, a Plasmodium protein, or a Toxoplasma protein.

In one aspect, the antigenic protein is from a prokaryotic organism, including gram positive bacteria, gram negative bacteria, or other bacteria, such as Bacillus (e.g., Bacillus anthracis), Mycobacterium (e.g., Mycobacterium tuberculosis, Mycobacterium Leprae), Shigella (e.g., Shigella sonnei, Shigella dysenteriae, Shigella flexneri), Helicobacter (e.g., Helicobacter pylori), Salmonella (e.g., Salmonella enterica, Salmonella typhi, Salmonella typhimurium), Neisseria (e.g., Neisseria gonorrhoeae, Neisseria meningitidis), Moraxella (e.g., Moraxella catarrhalis), Haemophilus (e.g., Haemophilus influenzae), Klebsiella (e.g., Klebsiella pneumoniae), Legionella (e.g., Legionella pneumophila), Pseudomonas (e.g., Pseudomonas aeruginosa), Acinetobacter (e.g., Acinetobacter baumannii), Listeria (e.g., Listeria monocytogenes), Staphylococcus (e.g., Staphylococcus aureus), Streptococcus (e.g., Streptococcus pneumoniae, Streptococcus pyogenes, Streptococcus agalactiae), Corynebacterium (e.g., Corynebacterium diphtheria), Clostridium (e.g., Clostridium botulinum, Clostridium tetani, Clostridium difficile), Chlamydia (e.g., Chlamydia pneumonia, Chlamydia trachomatis), Caphylobacter (e.g., Caphylobacter jejuni), Bordetella (e.g., Bordetella pertussis), Enterococcus (e.g., Enterococcus faecalis, Enterococcus faecum), Vibrio (e.g., Vibrio cholerae), Yersinia (e.g., Yersinia pestis), Burkholderia (e.g., Burkholderia cepacia complex), Coxiella (e.g., Coxiella burnetti), Francisella (e.g., Francisella tularensis), and Escherichia (e.g., enterotoxigenic, enterohemorrhagic or Shiga toxin-producing E. coli, such as ETEC, EHEC, EPEC, EIEC, and EAEC)). In another aspect, the antigenic protein is from a eukaryotic organism, including protists and fungi, such as Plasmodium (e.g., Plasmodium falciparum, Plasmodium vivax, Plasmodium ovale, Plasmodium malariae, Plasmodium diarrhea), Candida (e.g., Candida albicans), Aspergillus (e.g., Aspergillus fumigatus), Cryptococcus (e.g., Cryptococcus neoformans), Histoplasma (e.g., Histoplasma capsulatum), Pneumocystis (e.g., Pneumocystis jirovecii), and Coccidiodes (e.g., Coccidiodes immitis).

In some aspects, the viral protein encoded by transgenes included in RNA molecules provided herein is a coronavirus protein. In some embodiments, the antigenic protein is a SARS-CoV-2 protein.

In one aspect, the antigenic protein is a SARS-CoV-2 spike glycoprotein or a fragment thereof. In another aspect, the SARS-CoV-2 spike glycoprotein is a wild-type SARS-CoV-2 spike glycoprotein. In some aspects, the SARS-CoV-2 spike glycoprotein is prefusion stabilized. Prefusion stabilized SARS-CoV-2 glycoproteins can include K986P, V987P, or both K986P and V987P mutations. In some aspects, the SARS-Cov-2 spike glycoprotein is a variant spike glycoprotein. As used herein, the term “variant SARS-CoV-2 spike glycoprotein” refers to any spike glycoprotein other than that of the SARS-CoV-2 Wuhan isolate(s) that emerged in Wuhan, China in 2019 (Wu, F., Zhao, S., Yu, B. et al. A new coronavirus associated with human respiratory disease in China. Nature 579, 265-269 (2020). doi.org/10.1038/s41586-020-2008-3). Accordingly, as used herein, the terms “wild-type SARS-CoV-2 spike glycoprotein” and “SARS-CoV-2 spike glycoprotein, Wuhan,” for example, can be used interchangeably, unless context clearly indicates otherwise.

Exemplary variant SARS-CoV-2 spike glycoproteins include, without limitation, the Alpha (B.1.1.7; UK), Beta (B. 1.351; South Africa), Gamma (P. 1; Brazil), Delta (B.1.617.2; India), and Lambda (C.37; Peru) variants. Additional variants, including further variants of concern, can be found at, e.g., COVID-19 Weekly Epidemiological Update, Edition 44, 15 Jun. 2021 (who.int/publications/m/item/weekly-epidemiological-update-on-covid-19---15-june-2021). Any SARS-CoV-2 spike glycoprotein variant or a fragment thereof and any SARS-CoV-2 spike glycoprotein mutant protein or a fragment thereof can be encoded by second polynucleotides of RNA molecules provided herein. For example, the second polynucleotide of RNA molecules provided herein can encode a SARS-CoV-2 spike protein comprising one or more mutations as compared to a wild-type SARS-CoV-2 spike glycoprotein sequence. Mutations can include substitutions, deletions, insertions, and others. Mutations can be present at any position or at any combination of positions of a SARS-CoV-2 spike glycoprotein. Any number of substitutions, insertions, deletions, or combinations thereof, can be present at any one or more positions of a SARS-CoV-2 spike glycoprotein. As an example, substitutions can include a change of a wild-type amino acid at any position or at any combination of positions to any other amino acid or combination of any other amino acids. Exemplary mutations include mutations at positions 614, 936, 320, 477, 986, 987, 988, or any combination thereof. In one aspect, a SARS-CoV-2 spike glycoprotein or a fragment thereof encoded by transgenes of second polynucleotides included in nucleic acid molecules provided herein includes a D614G mutation, a D936Y mutation, a D936H mutation, a V320G mutation, an S477N mutation, an S477I mutation, an S477T mutation, a K986P mutation, a V987P mutation, or any combination thereof. Additional mutations and variants can be found in the National Bioinformatics Center 2019 Novel Coronavirus Information Database (2019nCoVR), National Genomics Data Center, China National Center for Bioinformation / Beijing Institute of Genomics, Chinese Academy of Science at bigd.big.ac.cn/ncov/variation/annotation.

Variant spike glycoproteins can also include proteins referred to as “VFLIP” spike glycoproteins, also designated “5P_FL2_DS3” (Olmedillas et al., Structure-based design of a highly stable, covalently-linked SARS-CoV-2 spike trimer with improved structural properties and immunogenicity, bioRxiv 2021.05.06.441046; doi.org/10.1101/2021.05.06.441046). Thus, any antigenic protein encoded by RNA molecules provided herein can be a VFLIP variant spike glycoprotein. Accordingly, variant spike glycoproteins can include 5 proline substitutions. Exemplary proline substitutions include V986P and V987P, and proline substitutions at positions 817, 892, 899, and 942 (Hsieh et al., 2020, Structure-Based Design of PrefusionStabilized SARS-CoV-2 Spikes. Science 369 (6510): 1501-5). Any combination of proline substitutions can be included in variant spike glycoproteins provided herein. In one aspect, variant spike glycoproteins include proline substitutions at positions 987, 817, 892, 899, and 942. Variant spike glycoproteins can also include a S1/S2 linker. Exemplary linkers include GP, GGGS (SEQ ID NO:318), GPGP (SEQ ID NO:319), and GGGSGGGS (SEQ ID NO:320). In one aspect, the linker is GGGSGGGS (SEQ ID NO:320). In another aspect, variant spike glycoproteins include proline substitutions at positions 987, 817, 892, 899, and 942 and further include a GGGSGGGS S1/S2 linker sequence (SEQ ID NO:320) and/or a disulfide bond Y707C-T883C (Olmedillas et al., Structure-based design of a highly stable, covalently-linked SARS-CoV-2 spike trimer with improved structural properties and immunogenicity, bioRxiv 2021.05.06.441046; doi.org/10.1101/2021.05.06.441046). Variant spike glycoproteins can also include a D614G substitution. Any combination of proline substitutions, linker sequence(s), disulfide bonds, and substitutions such as D614G can be included in variant spike glycoproteins provided herein. In one aspect, variant spike glycoproteins include proline substitutions at positions 987, 817, 892, 899, and 942, a GGGSGGGS S1/S2 linker sequence (SEQ ID NO:320), and a disulfide bond Y707C-T883C. In another aspect, variant spike glycoproteins include proline substitutions at positions 987, 817, 892, 899, and 942, a GGGSGGGS S1/S2 linker sequence (SEQ ID NO:320), a disulfide bond Y707C-T883C, and a D614G substitution. Transgenes encoding any variant spike glycoprotein described herein can be included in RNA molecules provided herein, such as self-replicating RNA and mRNA molecules. In one aspect, one or more transgenes encoding a variant spike glycoprotein that includes proline substitutions at positions 987, 817, 892, 899, and 942, a GGGSGGGS S1/S2 linker sequence (SEQ ID NO:320), and a disulfide bond Y707C-T883C is included in self-replicating RNA molecules provided herein. In another aspect, one or more transgenes encoding a variant spike glycoprotein that includes proline substitutions at positions 987, 817, 892, 899, and 942, a GGGSGGGS S1/S2 linker sequence (SEQ ID NO:320), and a disulfide bond Y707C-T883C is included in mRNA molecules provided herein. In yet another aspect, one or more transgenes encoding a variant spike glycoprotein that includes proline substitutions at positions 987, 817, 892, 899, and 942, a GGGSGGGS S1/S2 linker sequence (SEQ ID NO:320), a disulfide bond Y707C-T883C, and a D614G substitution is included in self-replicating RNA molecules provided herein. In yet a further aspect, one or more transgenes encoding a variant spike glycoprotein that includes proline substitutions at positions 987, 817, 892, 899, and 942, a GGGSGGGS S1/S2 linker sequence (SEQ ID NO:320), a disulfide bond Y707C-T883C, and a D614G substitution is included in mRNA molecules provided herein.

In some aspects, the variant SARS-CoV-2 spike glycoprotein encoded by second polynucleotides of RNA molecules provided herein has an amino acid sequence of SEQ ID NO:SEQ ID NO: 14, SEQ ID NO:15, SEQ ID NO:16, SEQ ID NO:17, SEQ ID NO:31, or SEQ ID NO:34. In yet another aspect, the second polynucleotide of RNA molecules provided herein encodes a SARS-VoV-2 spike glycoprotein sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, identity to a sequence of SEQ ID NO:SEQ ID NO: 14, SEQ ID NO:15, SEQ ID NO:16, SEQ ID NO:17, SEQ ID NO:31, or SEQ ID NO:34. In another aspect, the second polynucleotide of RNA molecules provided herein comprises a sequence of SEQ ID NO:10, SEQ ID NO: 11, SEQ ID NO:12, SEQ ID NO: 13, SEQ ID NO:30, or SEQ ID NO:33. In further aspects, first transgenes included in second polynucleotides of RNA molecules provided herein comprise a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to a sequence of SEQ ID NO:10, SEQ ID NO:11, SEQ ID NO:12, SEQ ID NO: 13, SEQ ID NO:30, or SEQ ID NO:33.

In one aspect, the antigenic protein encoded by first transgenes of second polynucleotides included in nucleic acid molecules provided herein is an influenza virus protein or a fragment thereof. In another aspect, the second polynucleotide includes one or more transgenes encoding one or more influenza virus proteins or fragments thereof. Exemplary influenza virus proteins that can be encoded by transgenes of second polynucleotides included in nucleic acid molecules provided herein include proteins from any human or animal virus, including influenza A virus, influenza B virus, influenza C virus, influenza D virus, or any combination thereof. Exemplary influenza proteins include hemagglutinin (HA), neuraminidase (NA), M2, M1, NP, NS1, NS2, PA, PB1, PB2, and PB1-F2. Hemagglutinin proteins from any influenza virus subtype, such as H1-H18 and any emerging hemagglutinin, and neuraminidase proteins from any influenza virus subtype, such as N1-N11 and any emerging neuraminidase, can be antigenic proteins encoded by transgenes included in second polynucleotides of nucleic acid molecules provided herein. Any suitable fragment of influenza virus proteins can be encoded by transgenes included in second polynucleotides of nucleic acid molecules provided herein, including, for example, one or more helper T lymphocyte (HTL) epitope, one or more cytotoxic T lymphocyte (CTL) epitope, or any combination thereof. In some aspects, first transgenes of second polynucleotides included in RNA molecules provided herein comprise a sequence of SEQ ID NO:46 or SEQ ID NO:52. In other aspects, first transgenes included in second polynucleotides of RNA molecules provided herein comprise a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to a sequence of SEQ ID NO:46 or SEQ ID NO:52. In further aspects, first transgenes of second polynucleotides included in RNA molecules provided herein encode a protein having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to a sequence of SEQ ID NO:47 or SEQ ID NO:53.

In some aspects, transgenes included in second polynucleotides of nucleic acid molecules provided herein encode a reporter or a marker, including selectable markers. Reporters and markers can include fluorescent proteins, such as green fluorescent protein (GFP), red fluorescent protein (RFP), yellow fluorescent protein (YFP), luciferase enzymes, such as firefly and Renilla luciferases, and antibiotic selection markers, for example.

In some aspects, the second polynucleotide of nucleic acid molecules provided herein comprises at least two transgenes. Any number of transgenes can be included in second polynucleotides of nucleic acid molecules provided herein, such as one, two, three, four, five, six, seven, eight, nine, ten, or more transgenes. In one aspect, the second polynucleotide of nucleic acid molecules provided herein includes a second transgene encoding a second antigenic protein or a fragment thereof or an immunomodulatory protein. In one aspect, the second polynucleotide further comprises an internal ribosomal entry site (IRES), a sequence encoding a 2A peptide, or a combination thereof, located between transgenes. As used herein, the term “2A peptide” refers to a small (generally 18-22 amino acids) sequence that allows for efficient, stoichiometric production of discrete protein products within a single reading frame through a ribosomal skipping event within the 2A peptide sequence. As used herein, the term “internal ribosomal entry site” or “IRES” refers to a nucleotide sequence that allows for the initiation of protein translation of a messenger RNA (mRNA) sequence in the absence of an AUG start codon or without using an AUG start codon. An IRES can be found anywhere in an mRNA sequence, such as at or near the beginning, at or near the middle, or at or near the end of the mRNA sequence, for example. In another aspect, the second polynucleotide further comprises a subgenomic promoter located between transgenes. The subgenomic promoter located between transgenes can be a further subgenomic promoter, such as a second, third, fourth, etc. subgenomic promoter located between second and third, third and fourth, fourth and fifth, etc. transgenes, for example.

Any number of transgenes included in second polynucleotides of nucleic acid molecules provided herein can be expressed via any combination of 2A peptide and IRES sequences. For example, a second transgene located 3′ of a first transgene can be expressed via a 2A peptide sequence or via an IRES sequence. As another example, a second transgene located 3′ of a first transgene and a third transgene located 3′ of the second transgene can be expressed via 2A peptide sequences located between the first and second transgenes and the second and third transgenes, via an IRES sequence located between the first and second transgenes and the second and third transgenes, via a 2A peptide sequence located between the first and second transgenes and an IRES located between the second and third transgenes, or via an IRES sequence located between the first and second transgenes and a 2A peptide sequence located between the second and third transgenes. Similar configurations and combinations of 2A peptide and IRES sequences located between transgenes are contemplated for any number of transgenes included in second polynucleotides of nucleic acid molecules provided herein. In addition to expression via 2A peptide and IRES sequences, two or more transgenes included in nucleic acid molecules provided herein can also be expressed from separate subgenomic RNAs.

A second, third, fourth, fifth, sixth, seventh, eighth, ninth, tenth, etc., transgene included in second polynucleotides of nucleic acid molecules provided herein can encode an immunomodulatory protein or a functional fragment or functional variant thereof. Any immunomodulatory protein or a functional fragment or functional variant thereof can be encoded by a transgene included in second polynucleotides.

As used herein, the terms “functional variant” or “functional fragment” refer to a molecule, including a nucleic acid or protein, for example, that comprises a nucleotide and/or amino acid sequence that is altered by one or more nucleotides and/or amino acids compared to the nucleotide and/or amino acid sequences of the parent or reference molecule. For a protein, a functional variant is still able to function in a manner that is similar to the parent molecule. In other words, the modifications in the amino acid and/or nucleotide sequence of the parent molecule do not significantly affect or alter the functional characteristics of the molecule encoded by the nucleotide sequence or containing the amino acid sequence. The functional variant may have conservative sequence modifications including nucleotide and amino acid substitutions, additions and deletions. These modifications can be introduced by standard techniques known in the art, such as site-directed mutagenesis and random PCR-mediated mutagenesis. Functional variants can also include, but are not limited to, derivatives that are substantially similar in primary structural sequence, but which contain, e.g., in vitro or in vivo modifications, chemical and/or biochemical, that are not found in the parent molecule. Such modifications include, inter alia, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI-anchor formation, hydroxylation, iodination, methylation, myristoylation, oxidation, pegylation, proteolytic processing, phosphorylation, prenylation, racemization, selenoylation, sulfation, transfer-RNA-mediated addition of amino acids to proteins such as arginylation, ubiquitination, and the like.

In one aspect, a second transgene included in second polynucleotides of nucleic acid molecules provided herein encodes a cytokine, a chemokine, or an interleukin. Exemplary cytokines include interferons, TNF-α, TGF-β, G-CSF, and GM-CSF. Exemplary chemokines include CCL3, CCL26, and CXCL7. Exemplary interleukins include IL-I, IL-2, IL-3, IL-4, IL-5, IL-6, IL-7, IL-8, IL-10, IL-12, IL-15, IL-18, IL-21, and IL-23. Any transgene or combination of transgenes encoding any cytokine, chemokine, interleukin, or combinations thereof, can be included in second polynucleotides of nucleic acid molecules provided herein.

In one aspect, first and second transgenes included in second polynucleotides of nucleic acid molecules provided herein encode viral proteins, bacterial proteins, fungal proteins, protozoan proteins, parasite proteins, immunomodulatory proteins, or any combination thereof. In yet another aspect, first, second, third, fourth, fifth, sixth, seventh, eighth, ninth, tenth, or more transgenes included in second polynucleotides of nucleic acid molecules provided herein encode viral proteins, bacterial proteins, fungal proteins, protozoan proteins, parasite proteins, immunomodulatory proteins, or any combination thereof.

In some aspects, the second transgene encodes a second coronavirus protein. In other aspects, the second transgene encodes a second influenza virus protein. In still other aspects, the first and second transgenes encode a coronavirus protein and an influenza virus protein, respectively. In further aspects, the first and second transgenes encode an influenza virus protein and a coronavirus protein, respectively.

### RNA and DNA Molecules

### RNA Molecules - Exemplary Features

Nucleic acid molecules provided herein can be DNA molecules or RNA molecules. It will be appreciated that T present in DNA is substituted with U in RNA, and vice versa. In one aspect, nucleic acid molecules provided herein are RNA molecules, wherein the first polynucleotide is located 5′ of the second polynucleotide. In another aspect, RNA molecules provided herein further include an intergenic region. The intergenic region can have at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to a sequence of SEQ ID NO:7 or to a sequence of SEQ ID NO:43.

RNA molecules provided herein can be self-replicating RNAs. In one aspect, RNA molecules provided herein include a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, and any number or range in between, or 100% identity to a sequence of SEQ ID NO:1, SEQ ID NO:2, SEQ ID NO:3, SEQ ID NO:4, or SEQ ID NO:40. RNA molecules provided herein can also be mRNAs. In some aspects, RNA molecules provided herein include a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to a sequence of SEQ ID NO:29, SEQ ID NO:32, or SEQ ID NO:48. It will be appreciated that T of sequences provided herein will be substituted with U in an RNA molecule.

An RNA molecule provided herein can be generated by in vitro transcription (IVT) of DNA molecules provided herein. In one aspect, RNA molecules provided herein are self-replicating RNA molecules. In another aspect, RNA molecules provided herein are mRNA molecules. In yet another aspect, RNA molecules provided herein further comprise a 5′ cap. Any 5′ cap can be included in RNA molecules provided herein, including 5′ caps having a Cap 1 structure, a Cap 1 (m6A) structure, a Cap 2 structure, or a Cap 0 structure. A population or plurality of RNA molecules provided herein can have the same 5′ cap or can have different 5′ caps. For example, a population or plurality of RNA molecules can have 5′ caps having a Cap 1 structure, a Cap 1 (m6A) structure, a Cap 2 structure, a Cap 0 structure, or any combination thereof.

In one aspect, RNA molecules provided herein include a 5′ cap having Cap 1 structure. In yet another aspect, RNA molecules provided herein are self-replicating RNA molecules comprising a 5′ cap having a Cap 1 structure. In a further aspect, RNA molecules provided herein comprise a cap having a Cap 1 structure, wherein a m7G is linked via a 5′-5′ triphosphate to the 5′ end of the 5′ UTR. In yet a further aspect, RNA molecules provided herein comprise a cap having a Cap 1 structure, wherein a m7G is linked via a 5′-5′ triphosphate to the 5′ end of the 5′ UTR comprising a sequence of SEQ ID NO:5 or SEQ ID NO:41. Any method of capping can be used, including, but not limited to using a Vaccinia Capping enzyme (New England Biolabs, Ipswich, Mass.) and co-transcriptional capping or capping at or shortly after initiation of in vitro transcription (IVT), by for example, including a capping agent as part of an in vitro transcription (IVT) reaction. (Nuc. Acids Symp. (2009) 53:129).

Only those RNA molecules, such mRNAs and self-replicating RNAs that can function as mRNAs, that carry the Cap structure are active in Cap dependent translation; “decapitation” of mRNA results in an almost complete loss of their template activity for protein synthesis (Nature, 255:33-37, (1975); J. Biol. Chem., vol. 253:5228-5231, (1978); and Proc. Natl. Acad. Sci. USA, 72:1189-1193, (1975)).

Another element of eukaryotic mRNA is the presence of 2′-O-methyl nucleoside residues at transcript position 1 (Cap 1), and in some cases, at transcript positions 1 and 2 (Cap 2). The 2′-O-methylation of mRNA provides higher efficacy of mRNA translation in vivo (Proc. Natl. Acad. Sci. USA, 77:3952-3956 (1980)) and further improves nuclease stability of the 5′-capped mRNA. The mRNA with Cap 1 (and Cap 2) is a distinctive mark that allows cells to recognize the bona fide mRNA 5′ end, and in some instances, to discriminate against transcripts emanating from infectious genetic elements (Nucleic Acid Research 43: 482-492 (2015)).

Some examples of 5′ cap structures and methods for preparing mRNAs comprising the same are given in WO2015/051169A2, WO/2015/061491, US 2018/0273576, and US Patent Nos. 8,093,367, 8,304,529, and U.S. 10,487,105. In some embodiments, the 5′ cap is m7GpppAmpG, which is known in the art. In some embodiments, the 5′ cap is m7GpppG or m7GpppGm, which are known in the art. Structural formulas for embodiments of 5′ cap structures are provided below.

In some embodiments, a self-replicating RNA or mRNA of the disclosure comprises a 5′ cap having the structure of Formula (Cap I).

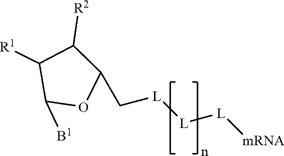

wherein B¹ is a natural or modified nucleobase; R¹ and R² are each independently selected from a halogen, OH, and OCH₃; each L is independently selected from the group consisting of phosphate, phophorothioate, and boranophosphate wherein each L is linked by diester bonds; n is 0 or 1. and mRNA represents an mRNA of the present disclosure linked at its 5′ end. In some embodiments B¹ is G, m⁷G, or A. In some embodiments, n is 0. In some embodiments n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, a self-replicating RNA or mRNA of the disclosure comprises a 5′ cap having the structure of Formula (Cap II).

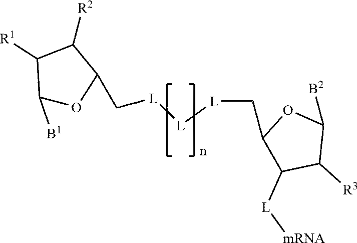

wherein B¹ and B² are each independently a natural or modified nucleobase; R¹, R², and R³ are each independently selected from a halogen, OH, and OCH₃, each L is independently selected from the group consisting of phosphate, phophorothioate, and boranophosphate wherein each L is linked by diester bonds; mRNA represents an mRNA of the present disclosure linked at its 5′ end; and n is 0 or 1.. In some embodiments B¹ is G, m⁷G, or A. In some embodiments, n is 0. In some embodiments, n is 1. In some embodiments, B¹ is A or m⁶A and R¹ is OCH₃; wherein G is guanine, m⁷G is 7-methylguanine, A is adenine, and m⁶A is N⁶-methyladenine.

In some embodiments, a self-replicating RNA or mRNA of the disclosure comprises a 5′ cap having the structure of Formula (Cap III).

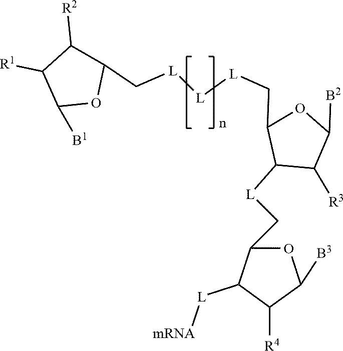

wherein B1, B2, and B3 are each independently a natural or modified nucleobase; R1, R2, R3, and R4 are each independently selected from a halogen, OH, and OCH3; each L is independently selected from the group consisting of phosphate, phosphorothioate, and boranophosphate wherein each L is linked by diester bonds; mRNA represents an mRNA of the present disclosure linked at its 5′ end; and n is 0 or 1.In some embodiments, at least one of R¹, R2, R3, and R4 is OH. In some embodiments B1 is G, m7G, or A. In some embodiments, B1 is A or m6A and R1 is OCH3; wherein G is guanine, m7G is 7-methylguanine, A is adenine, and m6A is N6-methyladenine. In some embodiments, n is 1.

In some embodiments, a self-replicating RNA or mRNA of the disclosure comprises a m7GpppG 5′ cap analog having the structure of Formula (Cap IV).

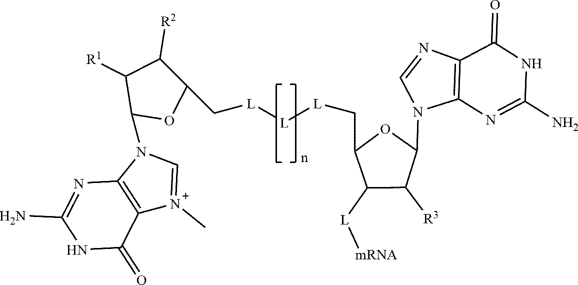

wherein, R¹, R², and R³ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; n is 0 or 1. In some embodiments, at least one of R¹, R², and R³ is OH. In some embodiments, the 5′ cap is m⁷GpppG wherein R¹, R², and R³ are each OH, n is 1, and each L is a phosphate. In some embodiments, n is 1. In some embodiments, the 5′ cap is m7GpppGm, wherein R¹ and R² are each OH, R³ is OCH₃, each L is a phosphate, and n is 1.

In some embodiments, a self-replicating RNA or mRNA of the disclosure comprises a m7Gpppm7G 5′ cap analog having the structure of Formula (Cap V).

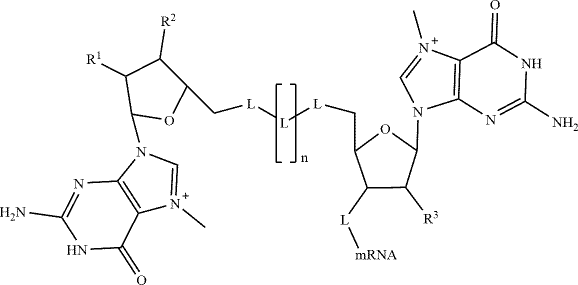

wherein, R¹, R², and R³ are each independently selected from a halogen, OH, and OCH₃; each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments, n is 1.

In some embodiments, a self-replicating RNA or mRNA of the disclosure comprises a m7Gpppm7GpN, 5′ cap analog, wherein N is a natural or modified nucleotide, the 5′ cap analog having the structure of Formula (Cap VI).

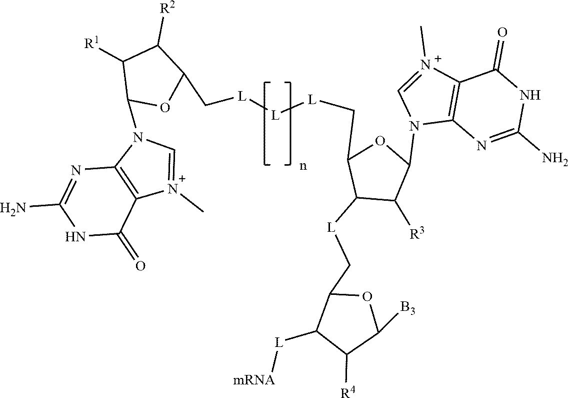

wherein B³ is a natural or modified nucleobase; R¹, R², R³, and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 3. In some embodiments, at least one of R¹, R², R³, and R⁴ is OH. In some embodiments B¹ is G, m⁷G, or A. In some embodiments, B¹ is A or m⁶A and R¹ is OCH₃; wherein G is guanine, m⁷G is 7-methylguanine, A is adenine, and m⁶A is N⁶-methyladenine. In some embodiments, n is 1.

In some embodiments, a self-replicating RNA or mRNA of the disclosure comprises a m7Gpppm7GpG 5′ cap analog having the structure of Formula (Cap VII).

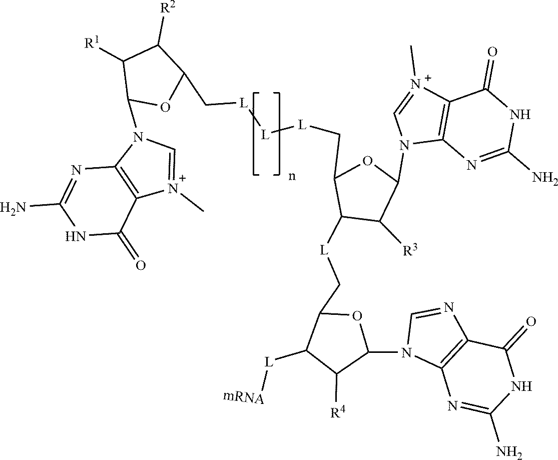

wherein, R¹, R², R³, and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments, n is 1.

In some embodiments, a self-replicating RNA or mRNA of the disclosure comprises a m7Gpppm7Gpm7G 5′ cap analog having the structure of Formula (Cap VIII).

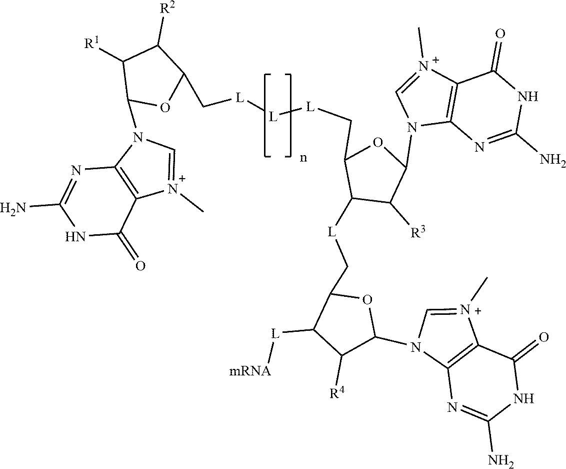

wherein, R¹, R², R³, and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; n is 0 or 1. In some embodiments, at least one of R¹, R², R³, and R⁴ is OH. In some embodiments, n is 1.

In some embodiments, a self-replicating RNA or mRNA of the disclosure comprises a m7GpppA 5′ cap analog having the structure of Formula (Cap IX).

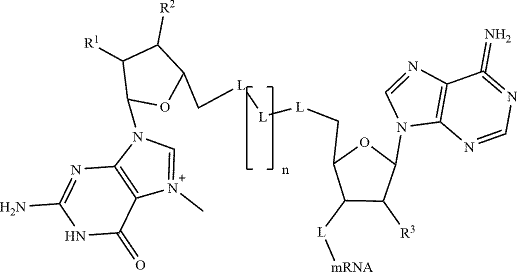

wherein, R¹, R², and R³ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R³ is OH. In some embodiments, n is 1.

In some embodiments, a self-replicating RNA or mRNA of the disclosure comprises a m7GpppApN 5′ cap analog, wherein N is a natural or modified nucleotide, and the 5′ cap has the structure of Formula (Cap X).

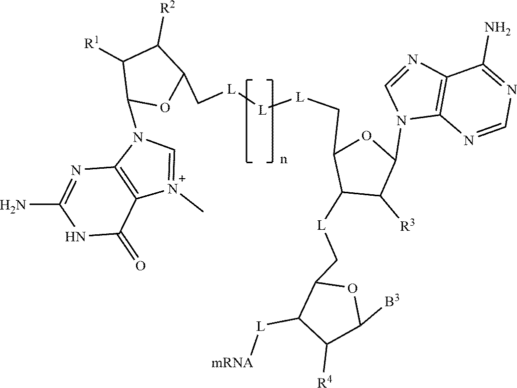

wherein B³ is a natural or modified nucleobase; R¹, R², R³, and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², R³, and R⁴ is OH. In some embodiments B³ is G, m⁷G, A or m⁶A; wherein G is guanine, m⁷G is 7-methylguanine, A is adenine, and m6A is N⁶-methyladenine. In some embodiments, n is 1.

In some embodiments, a self-replicating RNA or mRNA of the disclosure comprises a m7GpppAmpG 5′ cap analog having the structure of Formula (Cap XI).

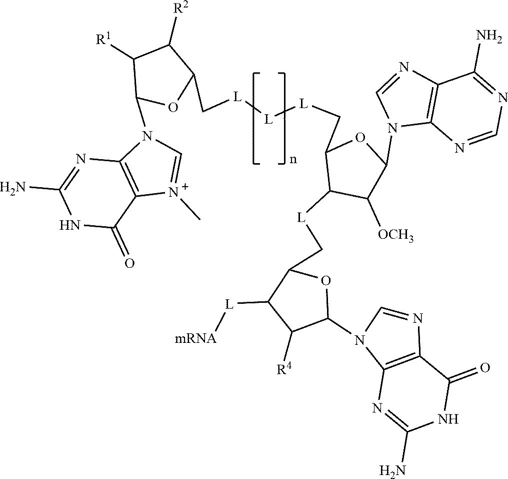

wherein, R¹, R², and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R⁴ is OH. In some embodiments, the compound of Formula Cap XI is m⁷GpppAmpG, wherein R¹, R², and R⁴ are each OH, n is 1, and each L is a phosphate linkage. In some embodiments, n is 1.

In some embodiments, a self-replicating RNA or mRNA of the disclosure comprises a m7GpppApm7G 5′ cap analog having the structure of Formula (Cap XII).

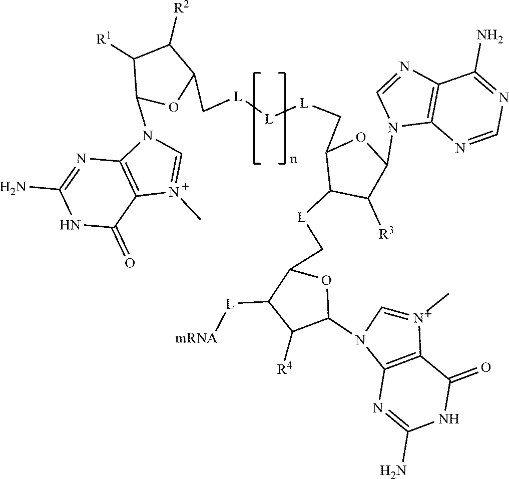

wherein, R¹, R², R³, and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or1. In some embodiments,at least one of R¹, R², R³, and R⁴ is OH. In some embodiments, n is 1.

In some embodiments, a self-replicating RNA or mRNA of the disclosure comprises a m7GpppApm7G 5′ cap analog having the structure of Formula (Cap XIII).

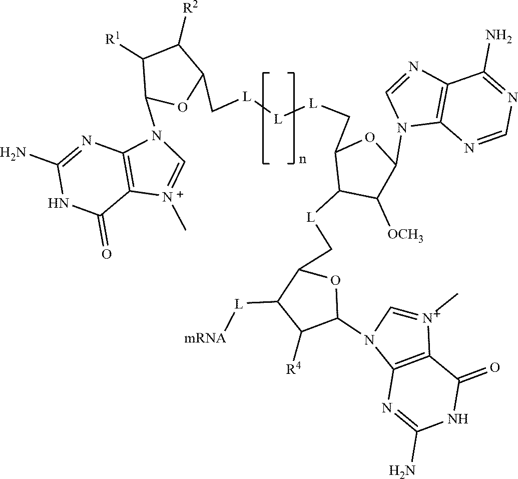

wherein, R¹, R², and R⁴ are each independently selected from a halogen, OH, and OCH₃, each L is independently selected from the group consisting of phosphate, phosphorothioate, and boranophosphate wherein each L is linked by diester bonds; mRNA represents an mRNA of the present disclosure linked at its 5′ end; and n is 0 or 1. In some embodiments, at least one of R¹, R², and R⁴ is OH. In some embodiments, n is 1.

### Poly-Adenine (Poly-A) Tail

Polyadenylation is the addition of a poly(A) tail, a chain of adenine nucleotides usually about 100-120 monomers in length, to a mRNA or an RNA that can function as an mRNA. In eukaryotes, polyadenylation is part of the process that produces mature mRNA for translation and begins as the transcription of a gene terminates. The 3′-most segment of a newly made pre-mRNA is first cleaved off by a set of proteins; these proteins then synthesize the poly(A) tail at the 3′ end. The poly(A) tail is important for the nuclear export, translation, and stability of mRNA. The tail is shortened over time, and, when it is short enough, the mRNA is enzymatically degraded. However, in a few cell types, mRNAs with short poly(A) tails are stored for later activation by re-polyadenylation in the cytosol.

Preferably, an RNA molecule of the disclosure comprises a 3′ tail region, which can serve to protect the RNA from exonuclease degradation. The tail region may be a 3′poly(A) and/or 3′poly(C) region. Preferably, the tail region is a 3′ poly(A) tail. Any self-replicating RNA and any mRNA, and any 3′ UTR of any self-replicating RNA or mRNA provided herein can include a poly(A) tail. As used herein a “3′ poly(A) tail” is a polymer of sequential adenine nucleotides that can range in size from, for example: 10 to 250 sequential adenine nucleotides; 60-125 sequential adenine nucleotides, 90-125 sequential adenine nucleotides, 95-125 sequential adenine nucleotides, 95-121 sequential adenine nucleotides, 100 to 121 sequential adenine nucleotides, 110-121 sequential adenine nucleotides; 112-121 sequential adenine nucleotides; 114-121 adenine sequential nucleotides; or 115 to 121 sequential adenine nucleotides. In some aspects, a 3′ poly(a) tail as described herein includes about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, 240, 250, 260, 270, 280, 290, 300, and any number or range in between, sequential adenine nucleotides. Preferably, a 3′ poly(A) tail as described herein comprises 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 sequential adenine nucleotides. 3′ Poly(A) tails can be added using a variety of methods known in the art, e.g., using poly(A) polymerase to add tails to synthetic or in vitro transcribed RNA. Other methods include the use of a transcription vector to encode poly(A) tails or the use of a ligase (e.g., via splint ligation using a T4 RNA ligase and/or T4 DNA ligase), wherein poly(A) may be ligated to the 3′ end of a sense RNA. In some embodiments, a combination of any of the above methods is utilized.

### DNA Molecules

In one aspect, provided herein are DNA molecules encoding the RNA molecules disclosed herein. In another aspect, DNA molecules provided herein further comprise a promoter. As used herein, the term “promoter” refers to a regulatory sequence that initiates transcription. A promoter can be operably linked to first and second polynucleotides of DNA molecules provided herein, with first and second polynucleotides of DNA molecules corresponding to encoded first and second polynucleotides of RNA molecules provided herein. Generally, promoters included in DNA molecules provided herein include promoters for in vitro transcription (IVT). Any suitable promoter for in vitro transcription can be included in DNA molecules provided herein, such as a T7 promoter, a T3 promoter, an SP6 promoter, and others. In one aspect, DNA molecules provided herein comprise a T7 promoter. In another aspect, the promoter is located 5′ of the 5′ UTR included in DNA molecules provided herein. In yet another aspect, the promoter is a T7 promoter located 5′ of the 5′ UTR included in DNA molecules provided herein. In yet another aspect, the promoter overlaps with the 5′ UTR. A promoter and a 5′ UTR can overlap by about one nucleotide, about two nucleotides, about three nucleotides, about four nucleotides, about five nucleotides, about six nucleotides, about seven nucleotides, about eight nucleotides, about nine nucleotides, about ten nucleotides, about , about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, about 50 nucleotides, or more nucleotides.

In some aspects, DNA molecules provided herein include a promoter for in vivo transcription. Generally, the promoter for in vivo transcription is an RNA polymerase II (RNA pol II) promoter. Any RNA pol II promoter can be included in DNA molecules provided herein, including constitutive promoters, inducible promoters, and tissue-specific promoters. Exemplary constitutive promoters include a cytomegalovirus (CMV) promoter, an EF1α promoter, an SV40 promoter, a PGK1 promoter, a Ubc promoter, a human beta actin promoter, a CAG promoter, and others. Any tissue-specific promoter can be included in DNA molecules provided herein. In one aspect, the RNA pol II promoter is a muscle-specific promoter, skin-specific promoter, subcutaneous tissue-specific promoter, liver-specific promoter, spleen-specific promoter, lymph node-specific promoter, or a promoter with any other tissue specificity. DNA molecules provided herein can also include an enhancer. Any enhancer that increases transcription can be included in DNA molecules provided herein.

### Design and Synthesis of RNA and DNA Molecules

RNA molecules provided herein can include any combination of the RNA sequences provided herein, including, for example, any 5′ UTR sequences, any sequences encoding a polyprotein that includes nsP1, nsP2, nsP3, and nsP4, any sequences encoding any transgene, and any 3′ UTR sequences provided herein. In some aspects, RNA molecules provided herein are self-replicating RNA molecules. Self-replicating RNA molecules can include sequences encoding a polyprotein that includes nsP1, nsP2, nsP3, and nsP4, for example. In some aspects, RNA molecules provided herein are mRNA molecules. Generally, mRNA molecules do not include sequences encoding a polyprotein for replication of the RNA.

In some aspects, RNA molecules provided herein include modified nucleotides. For example, 0% to 100%, 1% to 100%, 25% to 100%, 50% to 100% and 75% to 100% of the uracil nucleotides of the RNA molecules can be modified. In some aspects, 1% to 100% of the uracil nucleotides are N1-methylpseudouridine or 5-methoxyuridine. In some embodiments,100% of the uracil nucleotides are N1-methylpseudouridine. In some embodiments, 100% of the uracil nucleotides are 5-methoxyuridine.

An RNA molecule, such as a self-replicating RNA or mRNA, of the disclosure may be obtained by any suitable means. Methods for the manufacture of RNA molecules are known in the art and would be readily apparent to a person of ordinary skill. An RNA molecule of the disclosure may be prepared according to any available technique including, but not limited to chemical synthesis, in vitro transcription (IVT) or enzymatic or chemical cleavage of a longer precursor, etc.

In some embodiments, an RNA molecule, such as a self-replicating RNA or mRNA, of the disclosure is produced from a primary complementary DNA (cDNA) construct. The cDNA constructs can be produced on an RNA template by the action of a reverse transcriptase (e.g., RNA-dependent DNA-polymerase). The process of design and synthesis of the primary cDNA constructs described herein generally includes the steps of gene construction, RNA production (either with or without modifications) and purification. In the IVT method, a target polynucleotide sequence encoding an RNA molecule of the disclosure is first selected for incorporation into a vector which will be amplified to produce a cDNA template. Optionally, the target polynucleotide sequence and/or any flanking sequences may be codon optimized. The cDNA template is then used to produce an RNA molecule of the disclosure through in vitro transcription (IVT). After production, the RNA molecule of the disclosure may undergo purification and clean-up processes. The steps of which are provided in more detail below.

The step of gene construction may include, but is not limited to gene synthesis, vector amplification, plasmid purification, plasmid linearization and clean-up, and cDNA template synthesis and clean-up. Once a protein of interest is selected for production, a primary construct is designed. Within the primary construct, a first region of linked nucleosides encoding the polypeptide of interest may be constructed using an open reading frame (ORF) of a selected nucleic acid (DNA or RNA) transcript. The ORF may comprise the wild type ORF, an isoform, variant or a fragment thereof. As used herein, an “open reading frame” or “ORF” is meant to refer to a nucleic acid sequence (DNA or RNA) which is can encode a polypeptide of interest. ORFs often begin with the start codon, ATG and end with a nonsense or termination codon or signal.

The cDNA templates may be transcribed to produce an RNA molecule of the disclosure using an in vitro transcription (IVT) system. The system typically comprises a transcription buffer, nucleotide triphosphates (NTPs), an RNase inhibitor and a polymerase. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate modified nucleic acids.

The primary cDNA template or transcribed RNA sequence may also undergo capping and/or tailing reactions. A capping reaction may be performed by methods known in the art to add a 5′ cap to the 5′ end of the primary construct. Methods for capping include, but are not limited to, using a Vaccinia Capping enzyme (New England Biolabs, Ipswich, Mass.) or capping at initiation of in vitro transcription, by for example, including a capping agent as part of the IVT reaction. (Nuc. Acids Symp. (2009) 53: 129). A poly(A) tailing reaction may be performed by methods known in the art, such as, but not limited to, 2′ O-methyltransferase and by methods as described herein. If the primary construct generated from cDNA does not include a poly-T, it may be beneficial to perform the poly(A)-tailing reaction before the primary construct is cleaned.

Codon optimized cDNA constructs encoding the non-structural proteins and the transgene for a self-replicating RNA are particularly suitable for generating self-replicating RNA sequences described herein. For example, such cDNA constructs may be used as the basis to transcribe, in vitro, a polyribonucleotide encoding a protein of interest as part of a self-replicating RNA. Codon optimized cDNA constructs can also be used to generate mRNAs provided herein.

The present disclosure also provides expression vectors comprising a nucleotide sequence encoding a self-replicating RNA or mRNA that is preferably operably linked to at least one regulatory sequence. Regulatory sequences are art-recognized and are selected to direct expression of the encoded polypeptide.

Accordingly, the term regulatory sequence includes promoters, enhancers, and other expression control elements. The design of the expression vector may depend on such factors as the choice of the host cell to be transformed and/or the type of protein desired to be expressed.

The present disclosure also provides polynucleotides (e.g. DNA, RNA, cDNA, mRNA, etc.) directed to a self-replicating RNA or mRNA of the disclosure that may be operably linked to one or more regulatory nucleotide sequences in an expression construct, such as a vector or plasmid. In certain embodiments, such constructs are DNA constructs. Regulatory nucleotide sequences will generally be appropriate for a host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the embodiments of the present disclosure. The promoters may be either naturally occurring promoters, or hybrid promoters that combine elements of more than one promoter.

An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

The present disclosure also provides a host cell transfected with a self-replicating RNA, mRNA, or DNA described herein. The self-replicating RNA, mRNA, or DNA can encode any protein of interest, for example an antigen, including the spike glycoprotein of the SARS-CoV-2 virus or any other viral glycoprotein, such as the influenza virus hemagglutinin and neuraminidase. The host cell may be any prokaryotic or eukaryotic cell. For example, a polypeptide encoded by a self-replicating RNA or mRNA may be expressed in bacterial cells such as E. coli, insect cells (e.g., using a baculovirus expression system), yeast, or mammalian cells. Other suitable host cells are known to those skilled in the art.

A host cell transfected with an expression vector comprising a self-replicating RNA or mRNA of the disclosure can be cultured under appropriate conditions to allow expression of the self-replicating RNA or mRNA and translation of the polypeptide to occur. Once expressed, a self-replicating RNA generally undergoes self-amplification and translation. The polypeptide may be secreted and isolated from a mixture of cells and medium containing the polypeptides. Alternatively, the polypeptides may be retained in the cytoplasm or in a membrane fraction and the cells harvested, lysed and the protein isolated. A cell culture includes host cells, media and other byproducts. Suitable media for cell culture are well known in the art.

The expressed proteins described herein can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide.

### Compositions and Pharmaceutical Compositions

Provided herein, in some embodiments, are compositions comprising any of the RNA or DNA molecules provided herein. Compositions provided herein can include a lipid. Any lipid can be included in compositions provided herein. In one aspect, the lipid is an ionizable cationic lipid. Any ionizable cationic lipid can be included in compositions comprising nucleic acid molecules provided herein.

The compositions and polynucleotides of the present disclosure may be used to immunize or vaccinate a subject against a viral infection. In some embodiments, the compositions and polynucleotides of the present disclosure may be used to vaccinate or immunize a subject against SARS-CoV-2, the virus causing COVID-19.

Also provided herein, in some embodiments, are pharmaceutical compositions comprising any of the RNA and DNA molecules provided herein and a lipid formulation. Any lipid can be included in lipid formulations of pharmaceutical compositions provided herein. In one aspect, lipid formulations of pharmaceutical compositions provided herein include an ionizable cationic lipid. Exemplary ionizable cationic lipids of compositions and pharmaceutical compositions provided herein include the following:

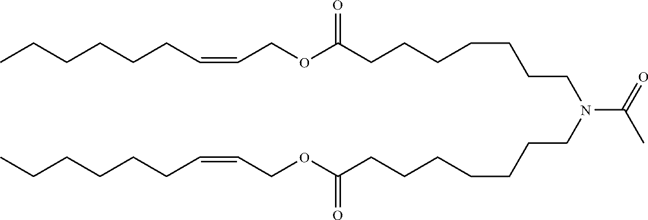 [SMILES: CCCCCC/C=C\COC(=O)CCCCCCCN(CCCCCCCC(=O)OC/C=C\CCCCCC)C(C)=O]

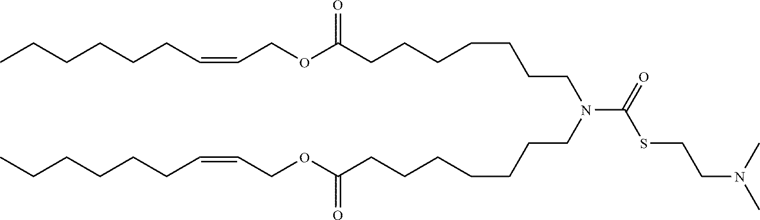 [SMILES: CCCCCC/C=C\COC(=O)CCCCCCCN(CCCCCCCC(=O)OC/C=C\CCCCCC)C(=O)SCCN(C)C]

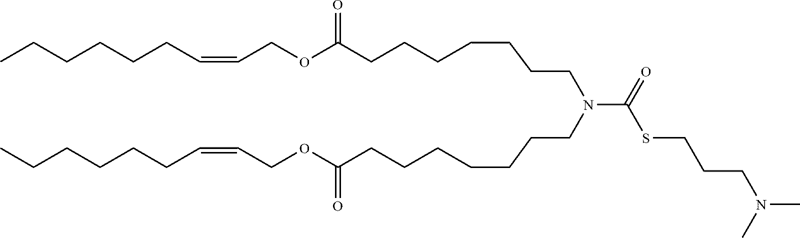 [SMILES: CCCCCC/C=C\COC(=O)CCCCCCCN(CCCCCCCC(=O)OC/C=C\CCCCCC)C(=O)SCCCN(C)C]

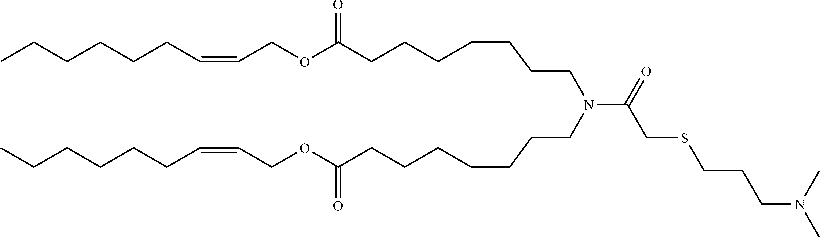 [SMILES: CCCCCC/C=C\COC(=O)CCCCCCCN(CCCCCCCC(=O)OC/C=C\CCCCCC)C(=O)CSCCCN(C)C]

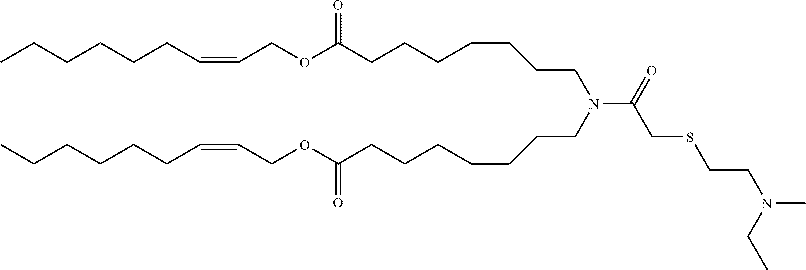 [SMILES: CCCCCC/C=C\COC(=O)CCCCCCCN(CCCCCCCC(=O)OC/C=C\CCCCCC)C(=O)CSCCN(C)CC]

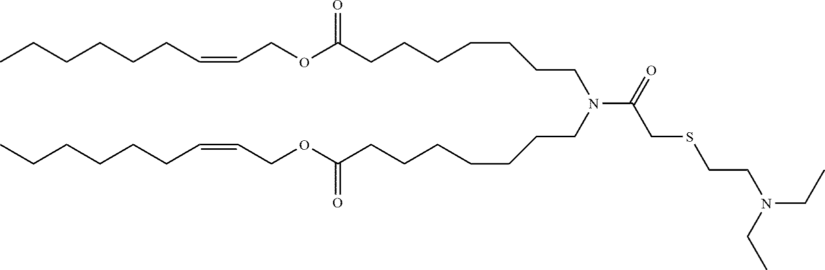 [SMILES: CCCCCC/C=C\COC(=O)CCCCCCCN(CCCCCCCC(=O)OC/C=C\CCCCCC)C(=O)CSCCN(CC)CC]

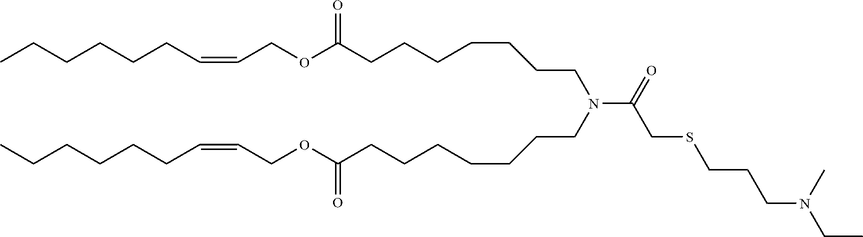 [SMILES: CCCCCC/C=C\COC(=O)CCCCCCCN(CCCCCCCC(=O)OC/C=C\CCCCCC)C(=O)CSCCCN(C)CC]

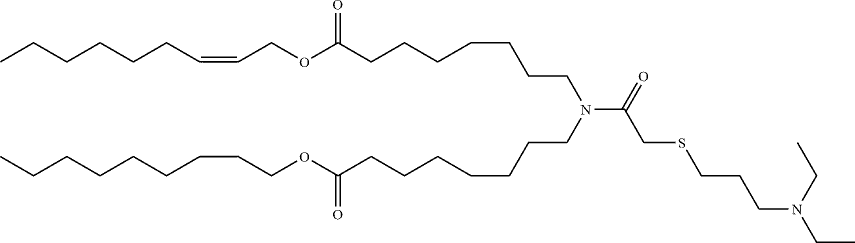 [SMILES: CCCCCC/C=C\COC(=O)CCCCCCCN(CCCCCCCC(=O)OCCCCCCCCC)C(=O)CSCCCN(CC)CC]

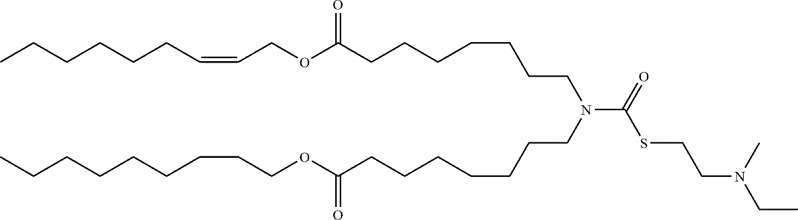 [SMILES: CCCCCC/C=C\COC(=O)CCCCCCCN(CCCCCCCC(=O)OCCCCCCCCC)C(=O)SCCN(C)CC]

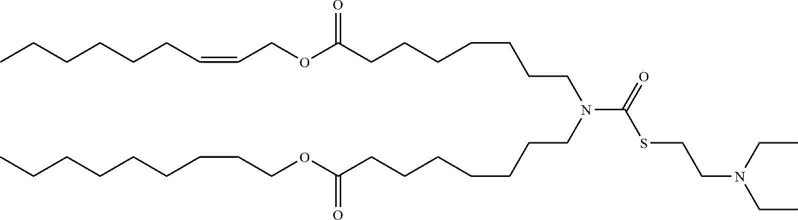 [SMILES: CCCCCC/C=C\COC(=O)CCCCCCCN(CCCCCCCC(=O)OCCCCCCCCC)C(=O)SCCN(CC)CC]

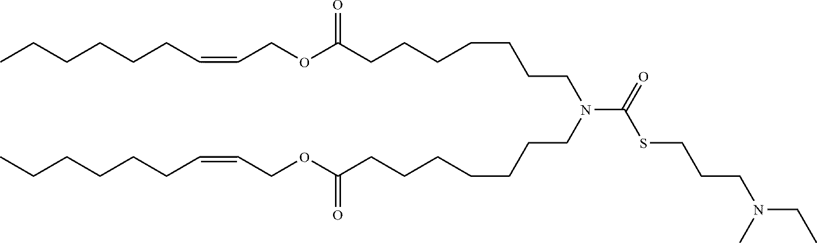 [SMILES: CCCCCC/C=C\COC(=O)CCCCCCCN(CCCCCCCC(=O)OC/C=C\CCCCCC)C(=O)SCCCN(C)CC]

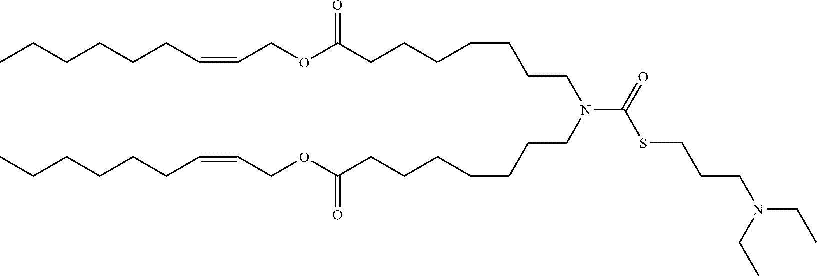 [SMILES: CCCCCC/C=C\COC(=O)CCCCCCCN(CCCCCCCC(=O)OC/C=C\CCCCCC)C(=O)SCCCN(CC)CC]

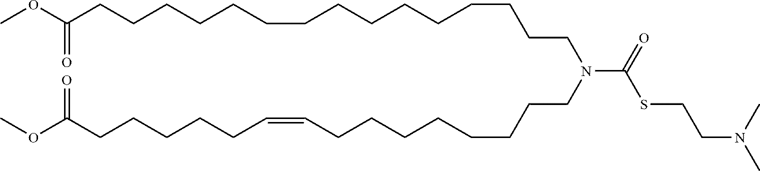

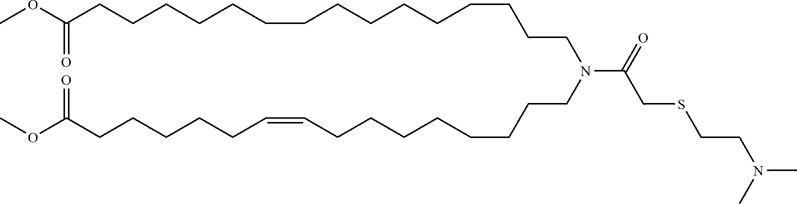

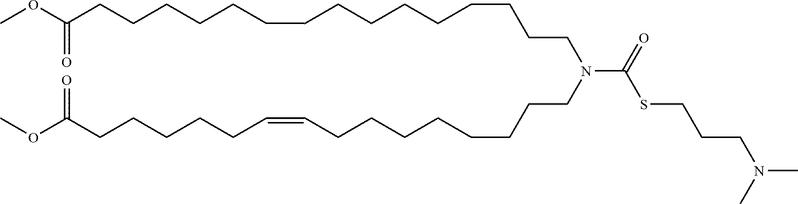

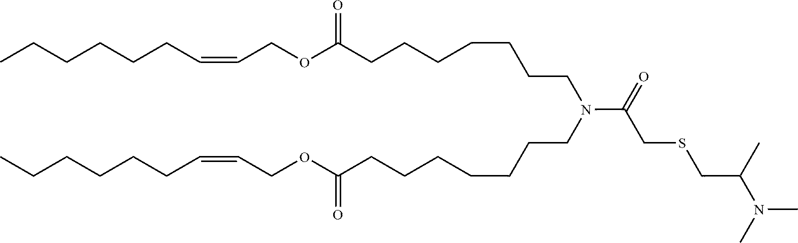 [SMILES: CCCCCC/C=C\COC(=O)CCCCCCCN(CCCCCCCC(=O)OC/C=C\CCCCCC)C(=O)CSCC(C)N(C)C]

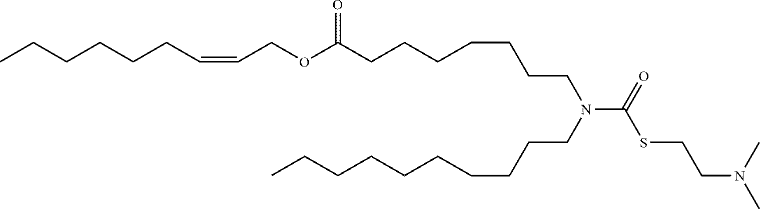 [SMILES: CCCCCC/C=C\COC(=O)CCCCCCCN(CCCCCCCCCC)C(=O)SCCN(C)C]

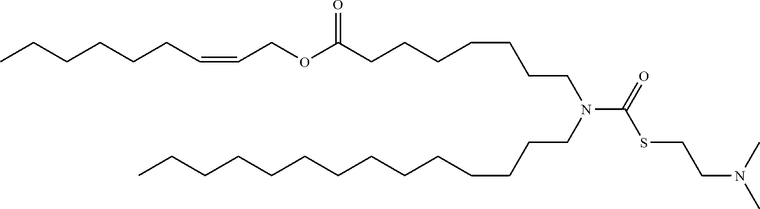 [SMILES: CCCCCC/C=C\COC(=O)CCCCCCCN(CCCCCCCCCCCCCC)C(=O)SCCN(C)C]

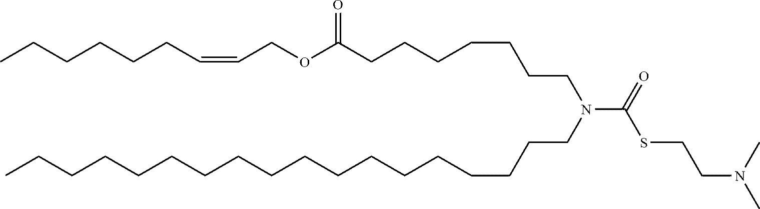 [SMILES: CCCCCC/C=C\COC(=O)CCCCCCCN(CCCCCCCCCCCCCCCCCC)C(=O)SCCN(C)C]

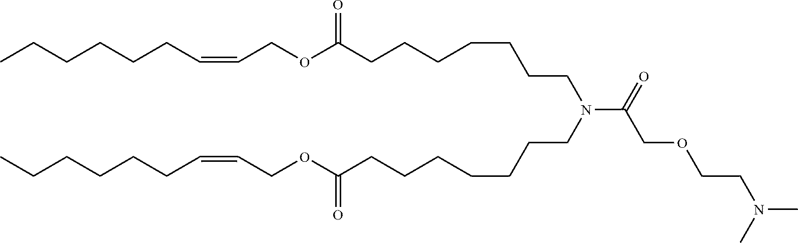 [SMILES: CCCCCC/C=C\COC(=O)CCCCCCCN(CCCCCCCC(=O)OC/C=C\CCCCCC)C(=O)COCCN(C)C]

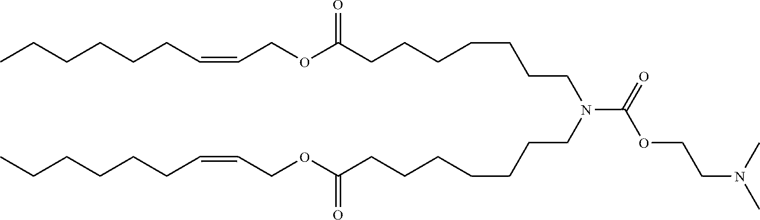 [SMILES: CCCCCC/C=C\COC(=O)CCCCCCCN(CCCCCCCC(=O)OC/C=C\CCCCCC)C(=O)OCCN(C)C]

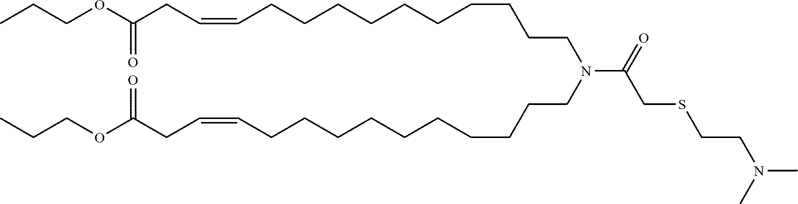 [SMILES: CCCOC(=O)C/C=C\CCCCCCCCCCN(CCCCCCCCCC/C=C\CC(=O)OCCC)C(=O)CSCCN(C)C]

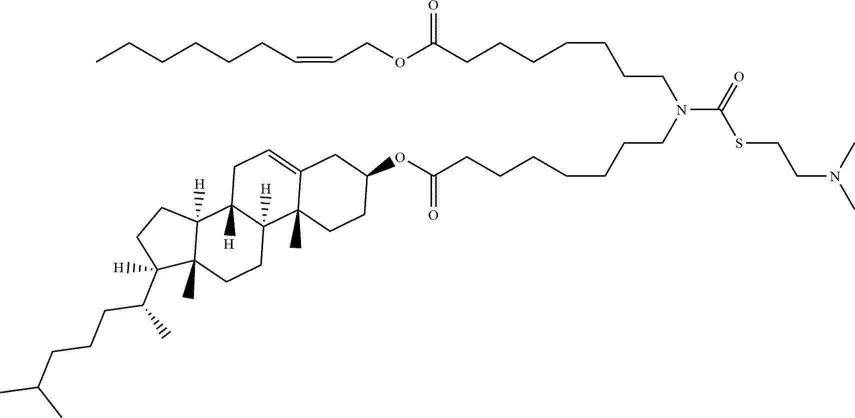

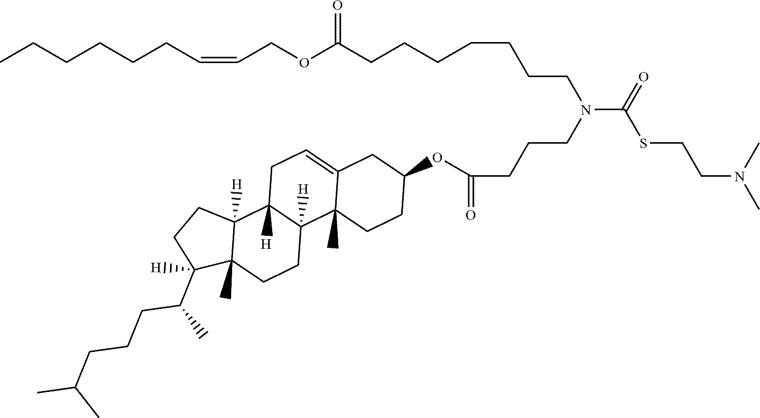

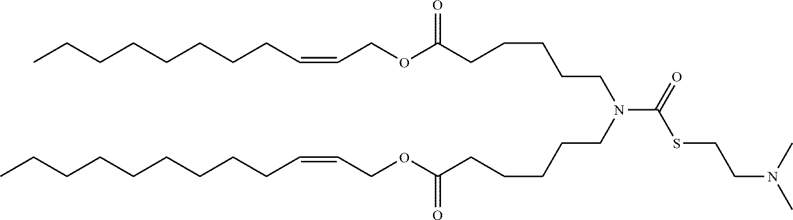 [SMILES: CCCCCCCC/C=C\COC(=O)CCCCCN(CCCCCC(=O)OC/C=C\CCCCCCCCC)C(=O)SCCN(C)C]

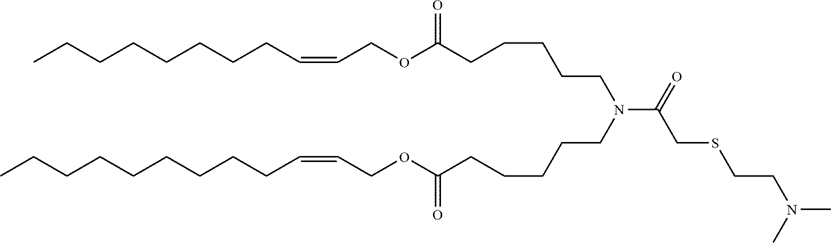 [SMILES: CCCCCCCC/C=C\COC(=O)CCCCCN(CCCCCC(=O)OC/C=C\CCCCCCCCC)C(=O)CSCCN(C)C]

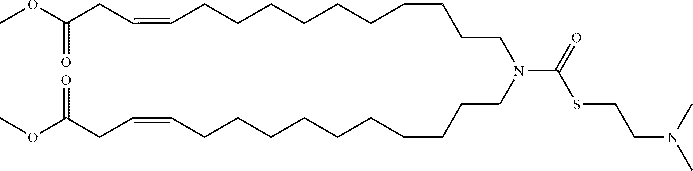

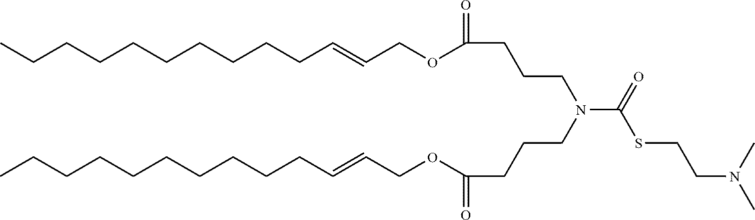 [SMILES: CCCCCCCCCC/C=C/COC(=O)CCCN(CCCC(=O)OC/C=C/CCCCCCCCCC)C(=O)SCCN(C)C]

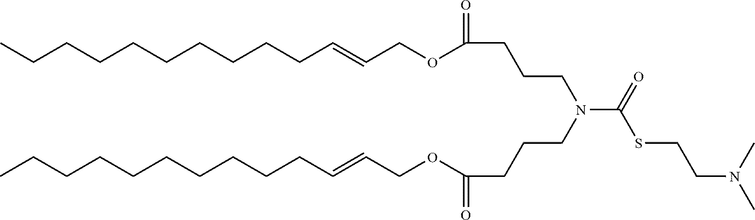 [SMILES: CCCCCCCCCC/C=C/COC(=O)CCCN(CCCC(=O)OC/C=C/CCCCCCCCCC)C(=O)SCCN(C)C]

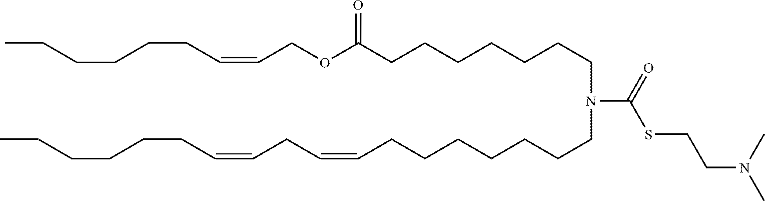 [SMILES: CCCCCC/C=C\C/C=C\CCCCCCCN(CCCCCCCC(=O)OC/C=C\CCCCCC)C(=O)SCCN(C)C]

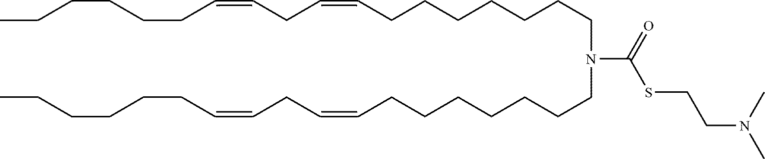 [SMILES: CCCCCC/C=C\C/C=C\CCCCCCCN(CCCCCCC/C=C\C/C=C\CCCCCC)C(=O)SCCN(C)C]

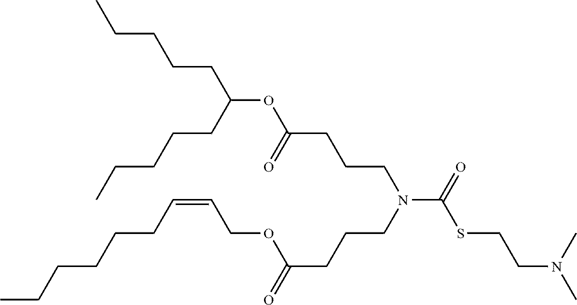 [SMILES: CCCCCC/C=C\COC(=O)CCCN(CCCC(=O)OC(CCCCC)CCCCC)C(=O)SCCN(C)C]

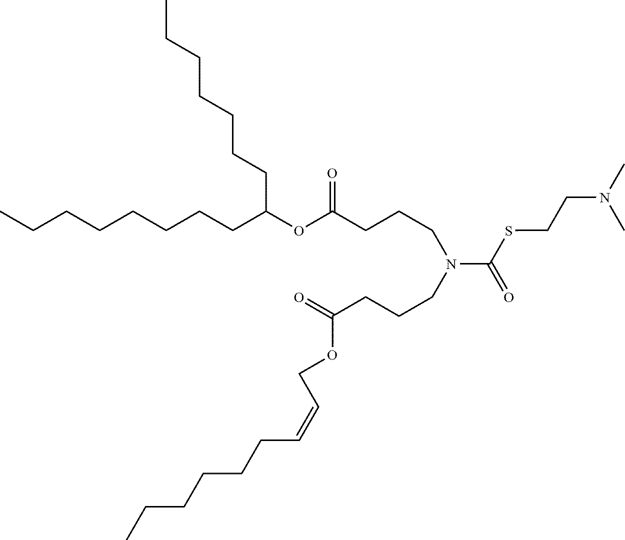 [SMILES: CCCCCC/C=C\COC(=O)CCCN(CCCC(=O)OC(CCCCCCC)CCCCCCCC)C(=O)SCCN(C)C]

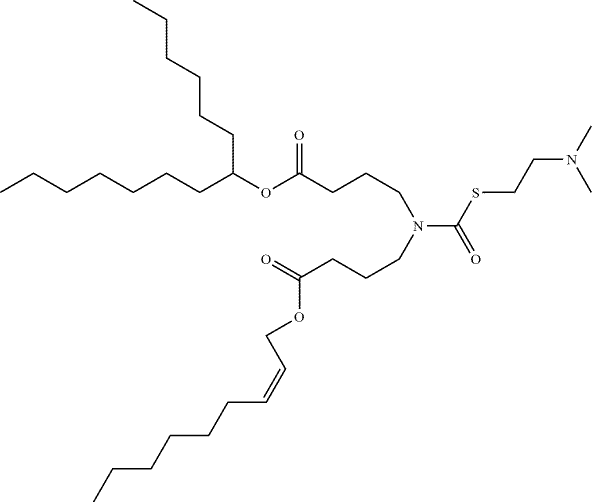 [SMILES: CCCCCC/C=C\COC(=O)CCCN(CCCC(=O)OC(CCCCCC)CCCCCCC)C(=O)SCCN(C)C]

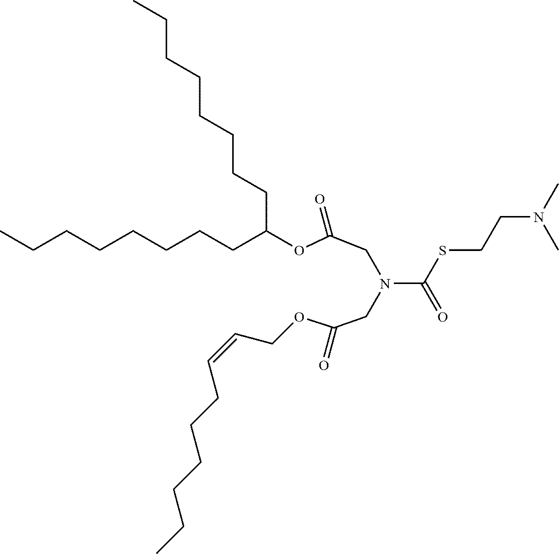 [SMILES: CCCCCC/C=C\COC(=O)CN(CC(=O)OC(CCCCCCCC)CCCCCCCC)C(=O)SCCN(C)C]

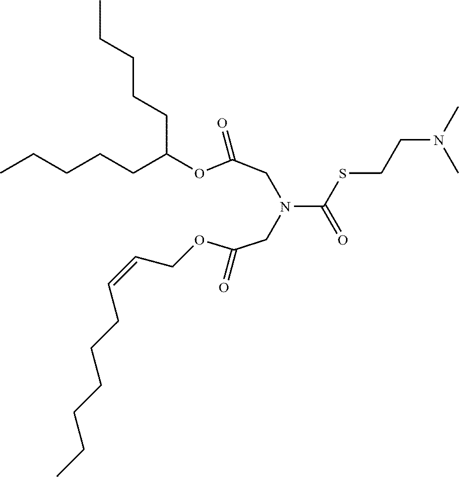 [SMILES: CCCCCC/C=C\COC(=O)CN(CC(=O)OC(CCCCC)CCCCC)C(=O)SCCN(C)C]

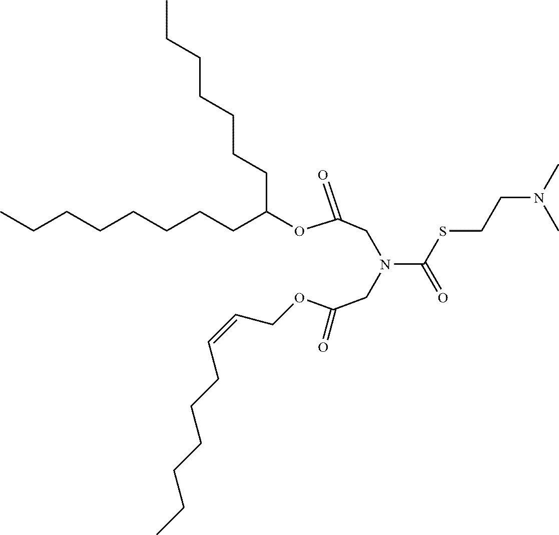 [SMILES: CCCCCC/C=C\COC(=O)CN(CC(=O)OC(CCCCCCC)CCCCCCCC)C(=O)SCCN(C)C]

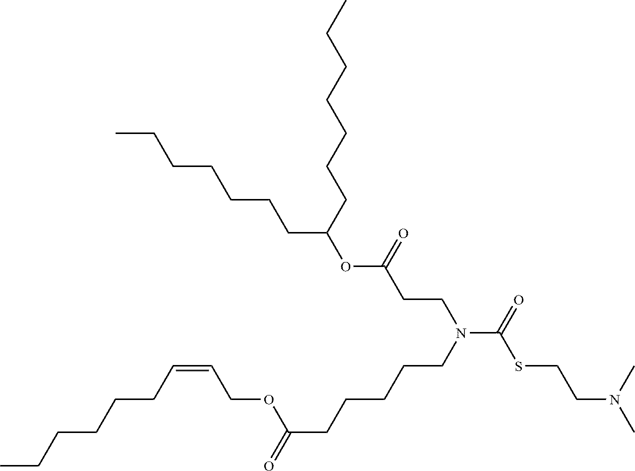 [SMILES: CCCCCC/C=C\COC(=O)CCCCCN(CCC(=O)OC(CCCCCCC)CCCCCCC)C(=O)SCCN(C)C]

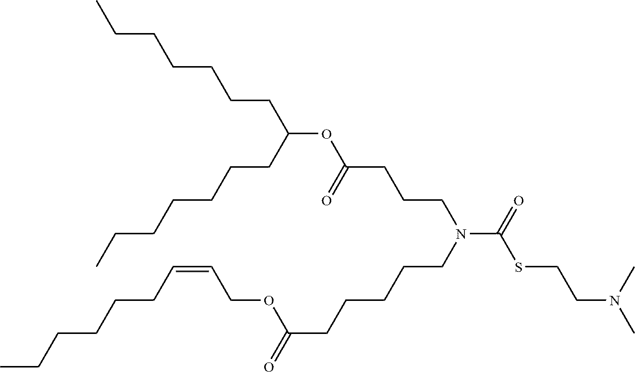 [SMILES: CCCCCC/C=C\COC(=O)CCCCCN(CCCC(=O)OC(CCCCCCC)CCCCCCC)C(=O)SCCN(C)C]

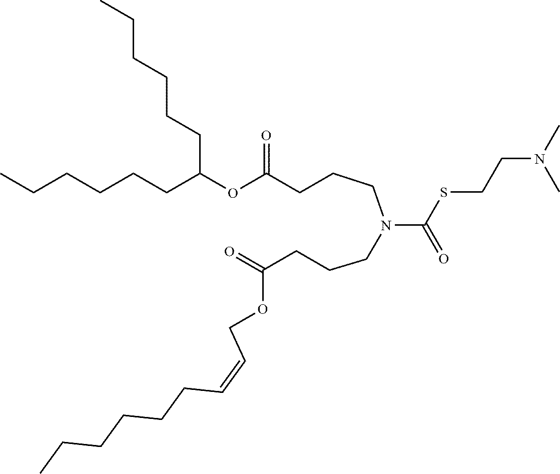 [SMILES: CCCCCC/C=C\COC(=O)CCCN(CCCC(=O)OC(CCCCCC)CCCCCC)C(=O)SCCN(C)C]

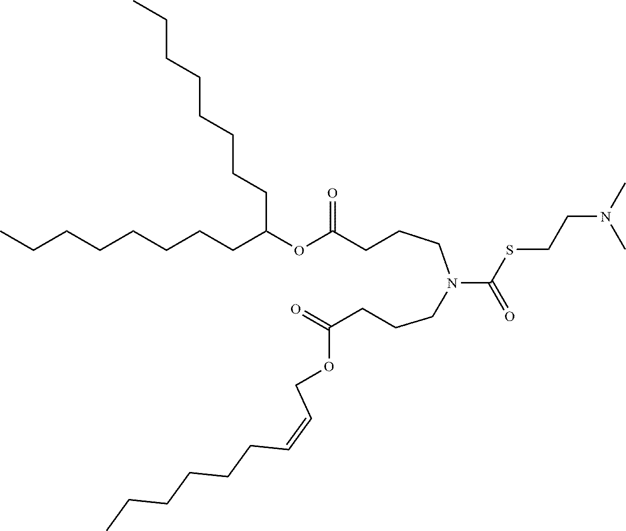 [SMILES: CCCCCC/C=C\COC(=O)CCCN(CCCC(=O)OC(CCCCCCCC)CCCCCCCC)C(=O)SCCN(C)C]

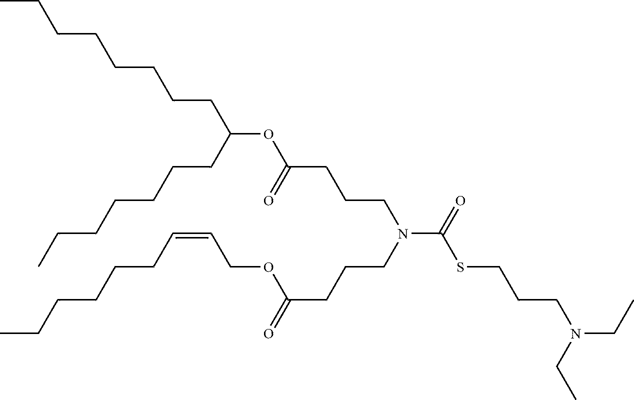 [SMILES: CCCCCC/C=C\COC(=O)CCCN(CCCC(=O)OC(CCCCCCC)CCCCCCCC)C(=O)SCCCN(CC)CC]

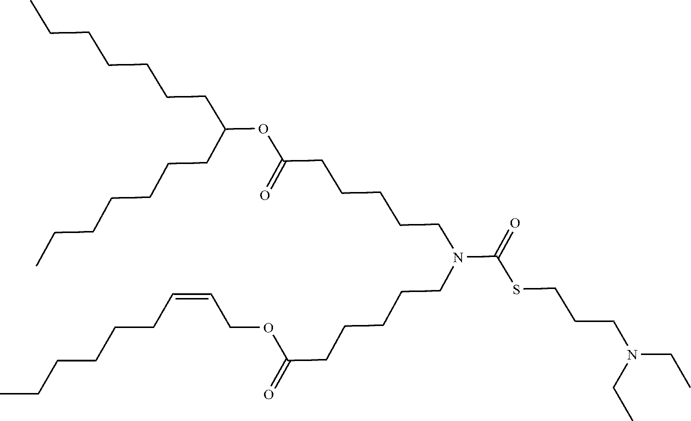 [SMILES: CCCCCC/C=C\COC(=O)CCCCCN(CCCCCC(=O)OC(CCCCCCC)CCCCCCC)C(=O)SCCCN(CC)CC]

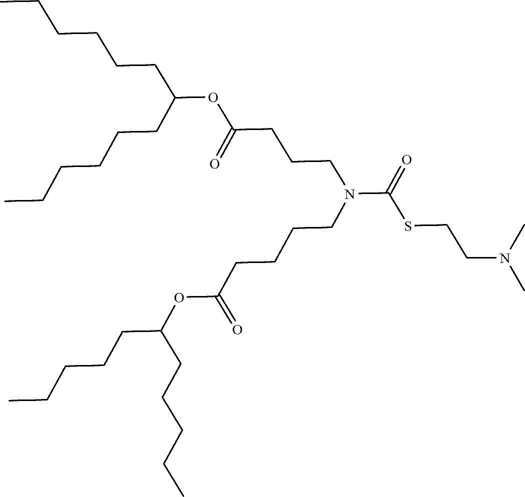 [SMILES: CCCCCCC(CCCCCC)OC(=O)CCCN(CCCCC(=O)OC(CCCCC)CCCCC)C(=O)SCCN(C)C]

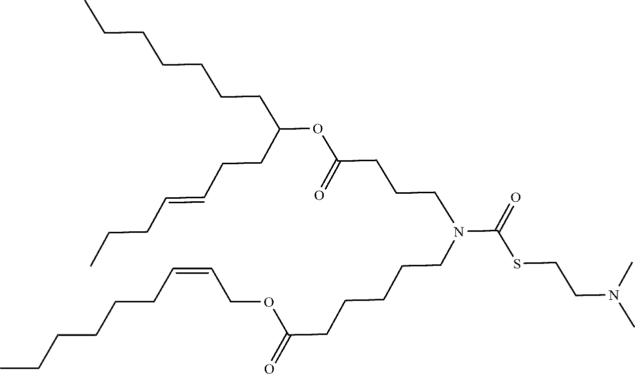 [SMILES: CCC/C=C/CCC(CCCCCCC)OC(=O)CCCN(CCCCCC(=O)OC/C=C\CCCCCC)C(=O)SCCN(C)C]

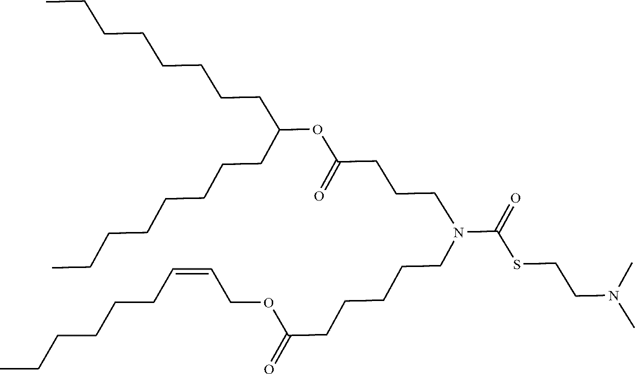 [SMILES: CCCCCC/C=C\COC(=O)CCCCCN(CCCC(=O)OC(CCCCCCCC)CCCCCCCC)C(=O)SCCN(C)C]

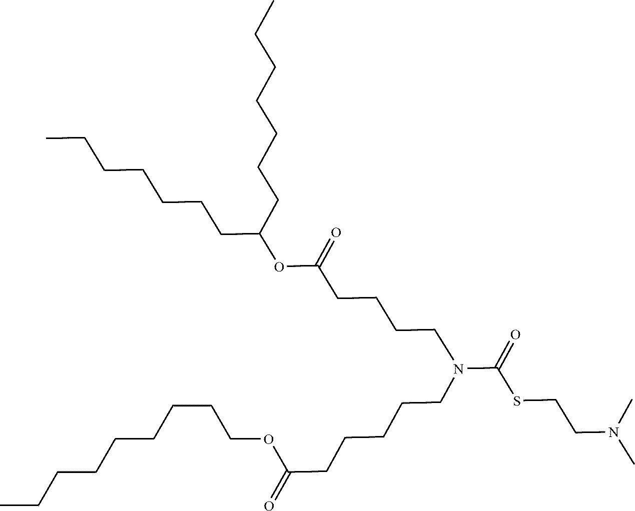 [SMILES: CCCCCCCCCOC(=O)CCCCCN(CCCCC(=O)OC(CCCCCCC)CCCCCCC)C(=O)SCCN(C)C]

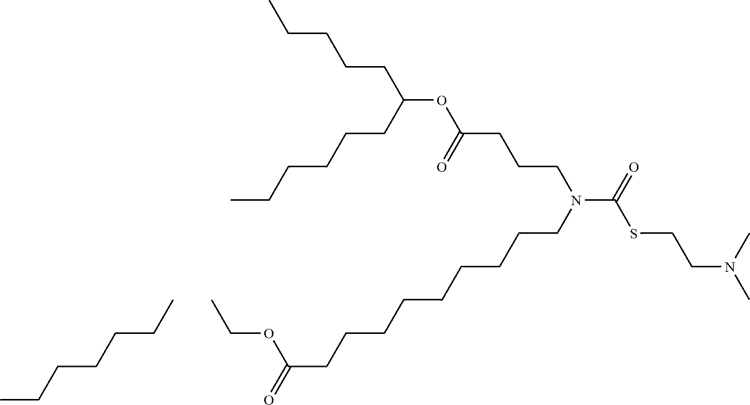

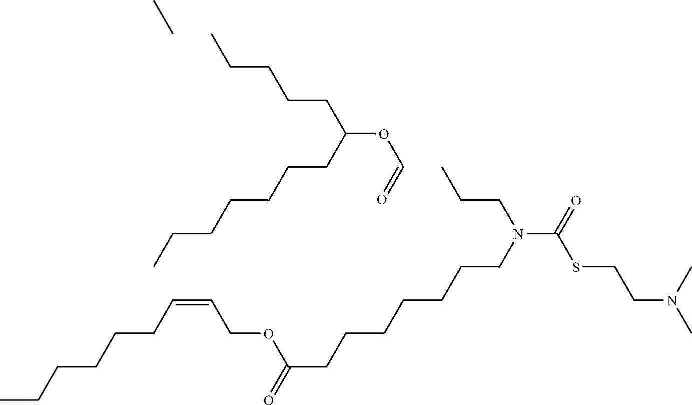

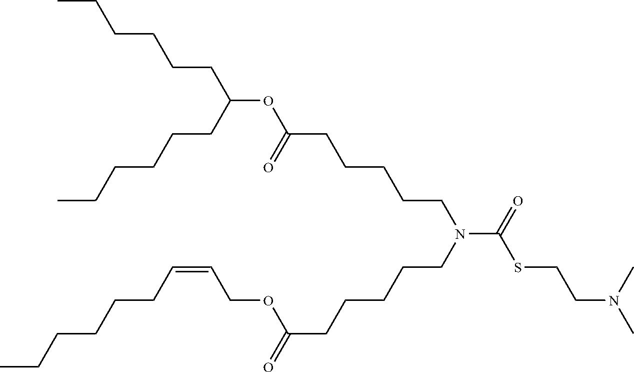 [SMILES: CCCCCC/C=C\COC(=O)CCCCCN(CCCCCC(=O)OC(CCCCCC)CCCCCC)C(=O)SCCN(C)C]

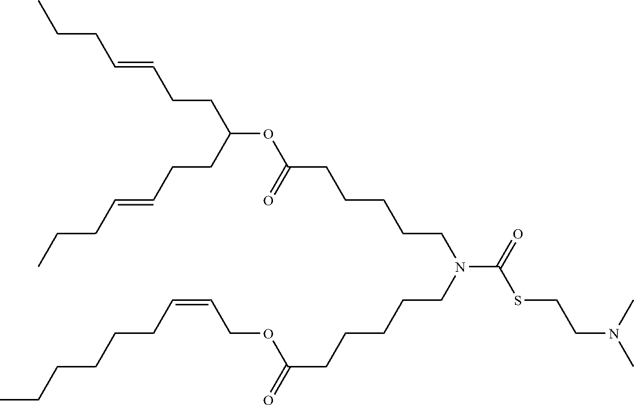 [SMILES: CCC/C=C/CCC(CC/C=C/CCC)OC(=O)CCCCCN(CCCCCC(=O)OC/C=C\CCCCCC)C(=O)SCCN(C)C]

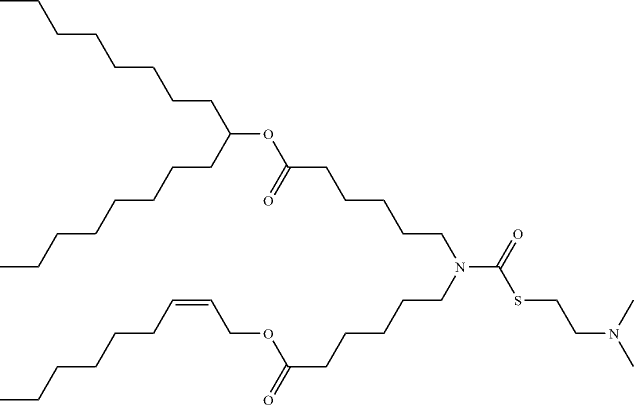 [SMILES: CCCCCC/C=C\COC(=O)CCCCCN(CCCCCC(=O)OC(CCCCCCCC)CCCCCCCC)C(=O)SCCN(C)C]

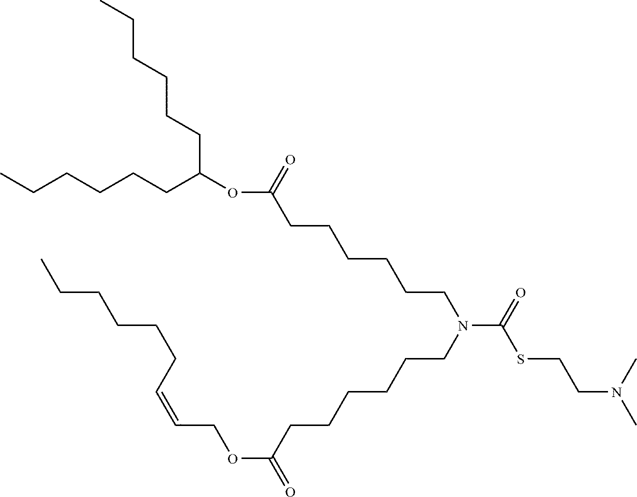 [SMILES: CCCCCC/C=C\COC(=O)CCCCCCN(CCCCCCC(=O)OC(CCCCCC)CCCCCC)C(=O)SCCN(C)C]

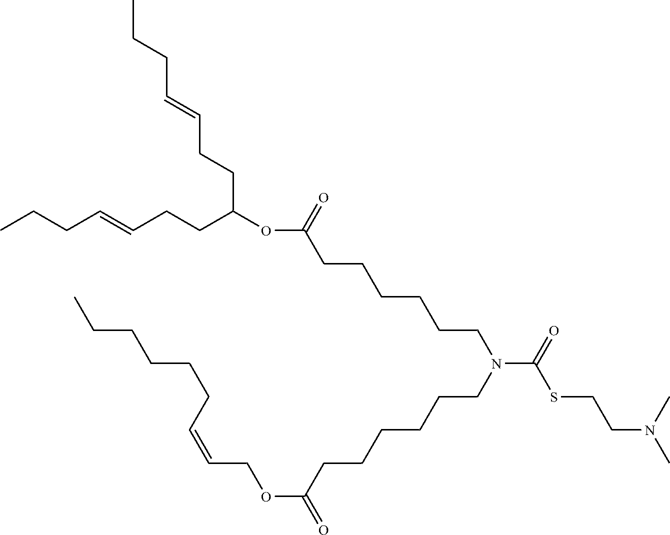 [SMILES: CCC/C=C/CCC(CC/C=C/CCC)OC(=O)CCCCCCN(CCCCCCC(=O)OC/C=C\CCCCCC)C(=O)SCCN(C)C]

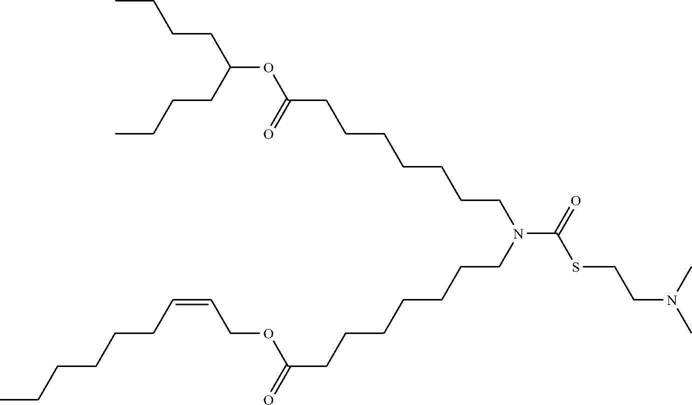 [SMILES: CCCCCC/C=C\COC(=O)CCCCCCCN(CCCCCCCC(=O)OC(CCCC)CCCC)C(=O)SCCN(C)C]

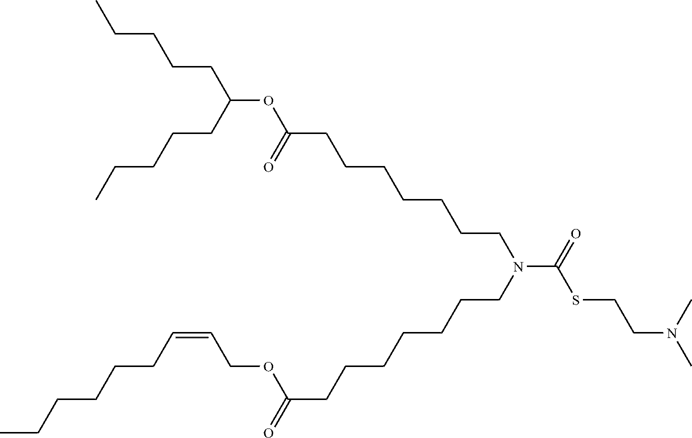 [SMILES: CCCCCC/C=C\COC(=O)CCCCCCCN(CCCCCCCC(=O)OC(CCCCC)CCCCC)C(=O)SCCN(C)C]

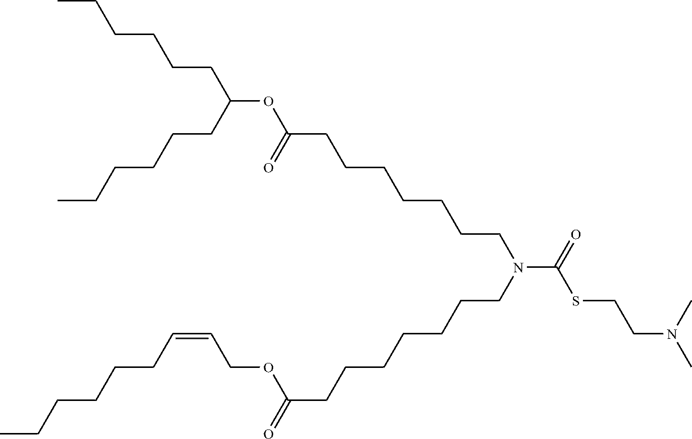 [SMILES: CCCCCC/C=C\COC(=O)CCCCCCCN(CCCCCCCC(=O)OC(CCCCCC)CCCCCC)C(=O)SCCN(C)C]

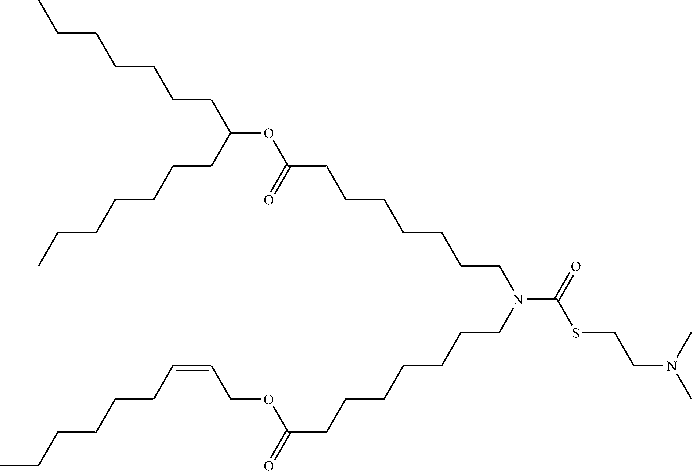 [SMILES: CCCCCC/C=C\COC(=O)CCCCCCCN(CCCCCCCC(=O)OC(CCCCCCC)CCCCCCC)C(=O)SCCN(C)C]

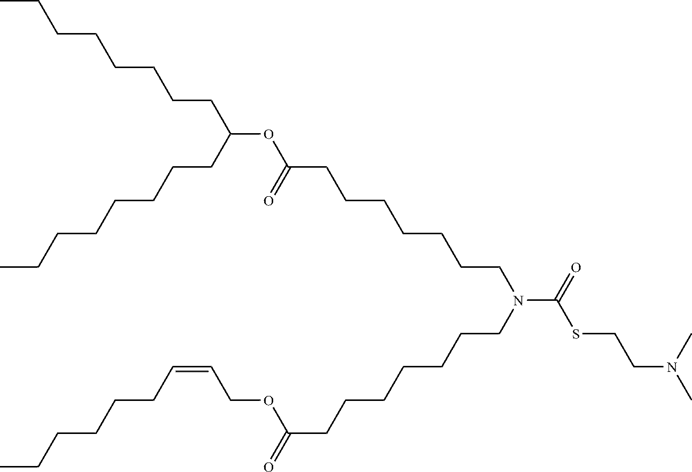 [SMILES: CCCCCC/C=C\COC(=O)CCCCCCCN(CCCCCCCC(=O)OC(CCCCCCCC)CCCCCCCC)C(=O)SCCN(C)C]

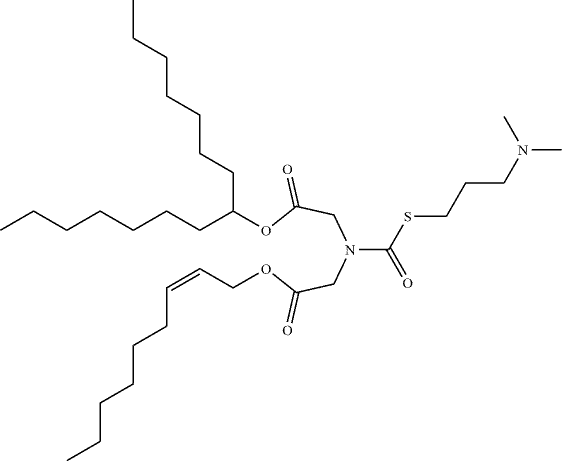 [SMILES: CCCCCC/C=C\COC(=O)CN(CC(=O)OC(CCCCCCC)CCCCCCC)C(=O)SCCCN(C)C]

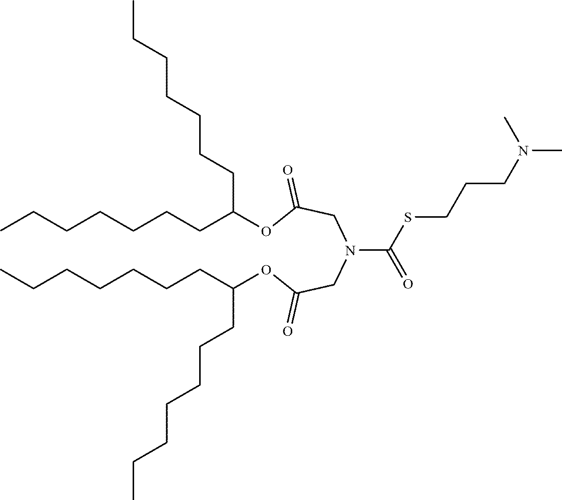 [SMILES: CCCCCCCC(CCCCCCC)OC(=O)CN(CC(=O)OC(CCCCCCC)CCCCCCC)C(=O)SCCCN(C)C]

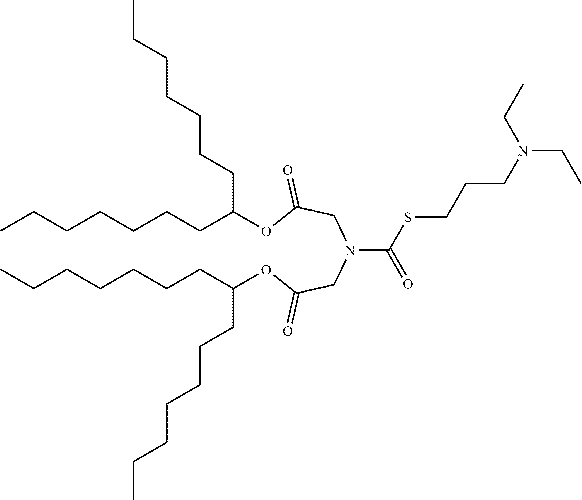 [SMILES: CCCCCCCC(CCCCCCC)OC(=O)CN(CC(=O)OC(CCCCCCC)CCCCCCC)C(=O)SCCCN(CC)CC]

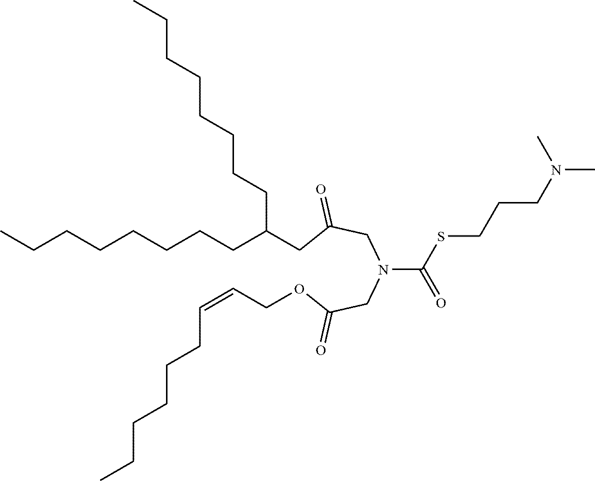 [SMILES: CCCCCC/C=C\COC(=O)CN(CC(=O)CC(CCCCCCCC)CCCCCCCC)C(=O)SCCCN(C)C]

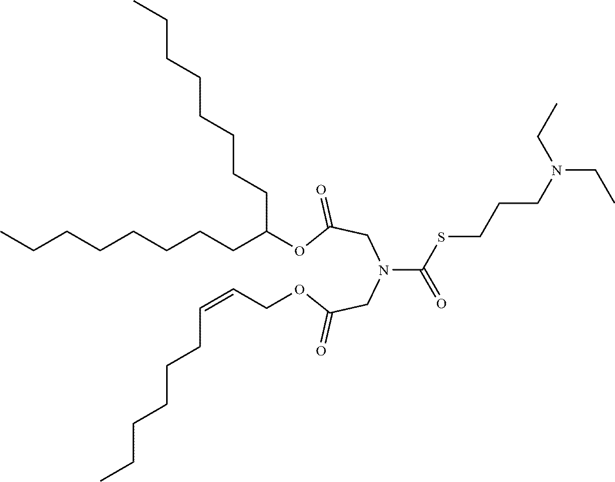 [SMILES: CCCCCC/C=C\COC(=O)CN(CC(=O)OC(CCCCCCCC)CCCCCCCC)C(=O)SCCCN(CC)CC]

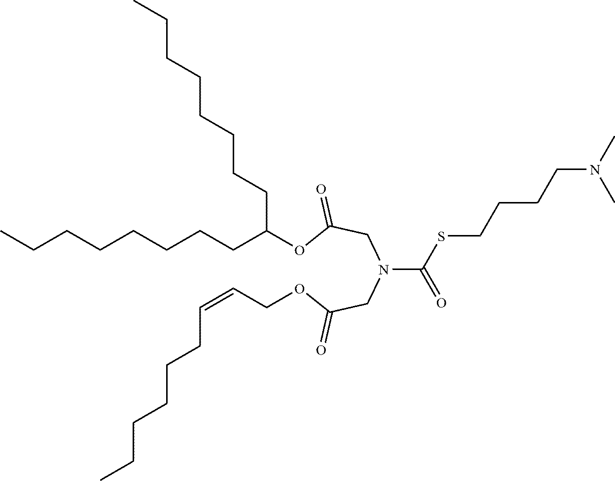 [SMILES: CCCCCC/C=C\COC(=O)CN(CC(=O)OC(CCCCCCCC)CCCCCCCC)C(=O)SCCCCN(C)C]

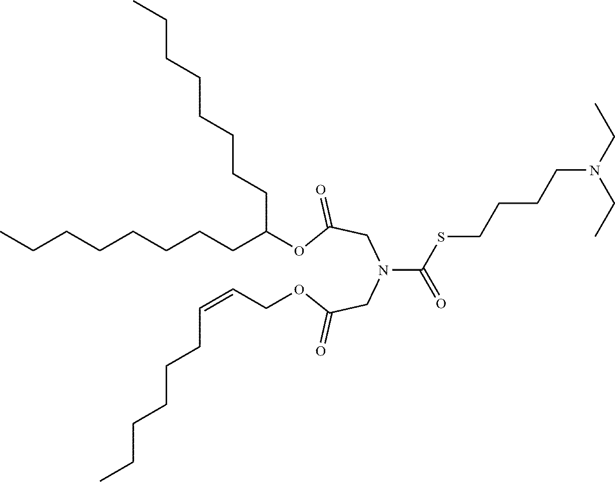 [SMILES: CCCCCC/C=C\COC(=O)CN(CC(=O)OC(CCCCCCCC)CCCCCCCC)C(=O)SCCCCN(CC)CC]

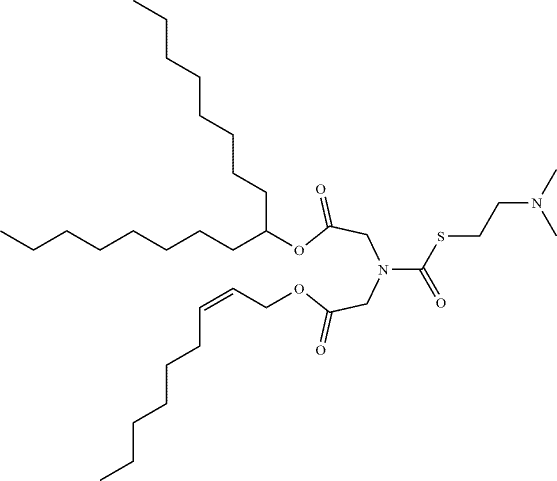 [SMILES: CCCCCC/C=C\COC(=O)CN(CC(=O)OC(CCCCCCCC)CCCCCCCC)C(=O)SCCN(C)C]

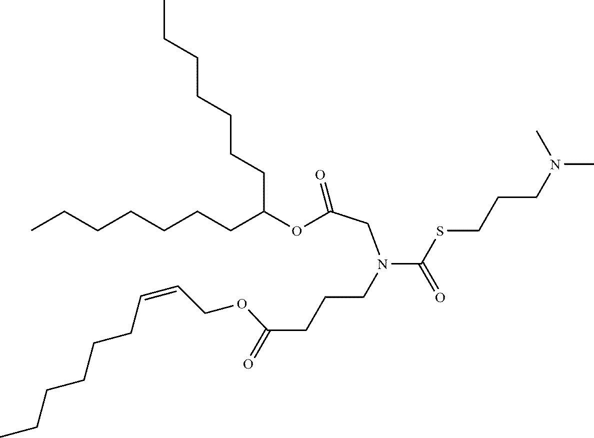 [SMILES: CCCCCC/C=C\COC(=O)CCCN(CC(=O)OC(CCCCCCC)CCCCCCC)C(=O)SCCCN(C)C]

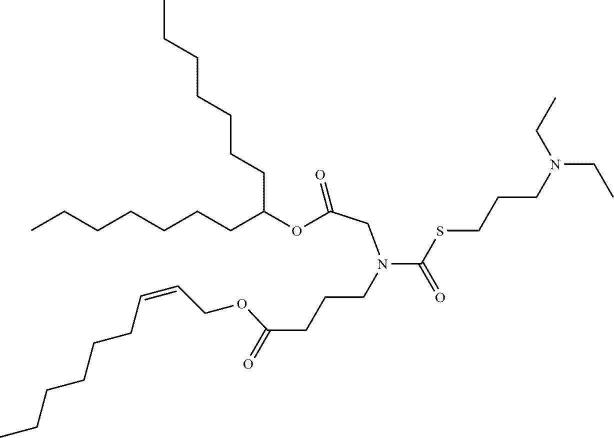 [SMILES: CCCCCC/C=C\COC(=O)CCCN(CC(=O)OC(CCCCCCC)CCCCCCC)C(=O)SCCCN(CC)CC]

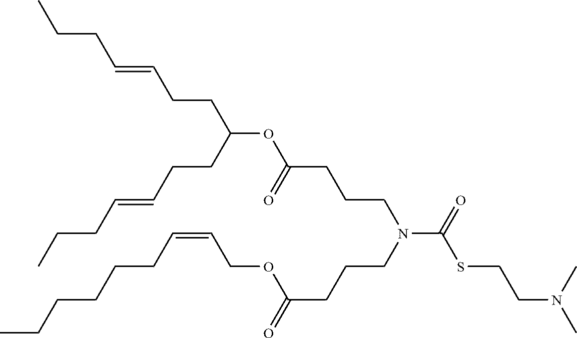 [SMILES: CCC/C=C/CCC(CC/C=C/CCC)OC(=O)CCCN(CCCC(=O)OC/C=C\CCCCCC)C(=O)SCCN(C)C]

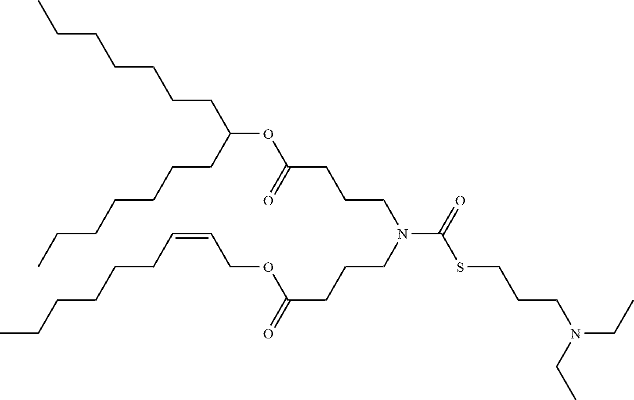 [SMILES: CCCCCC/C=C\COC(=O)CCCN(CCCC(=O)OC(CCCCCCC)CCCCCCC)C(=O)SCCCN(CC)CC]

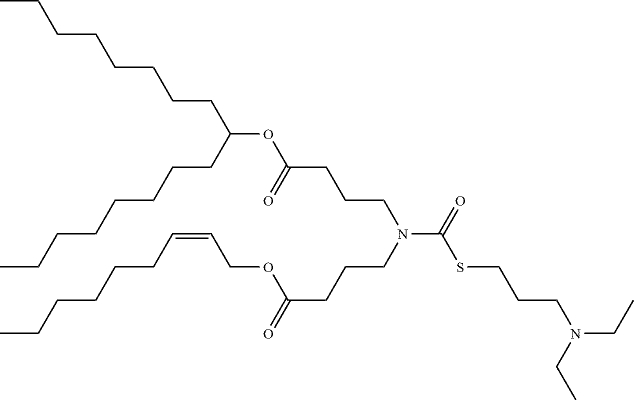 [SMILES: CCCCCC/C=C\COC(=O)CCCN(CCCC(=O)OC(CCCCCCCC)CCCCCCCC)C(=O)SCCCN(CC)CC]

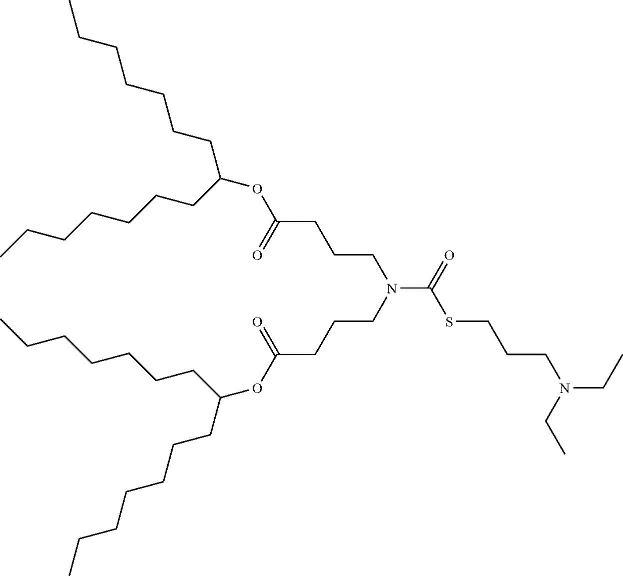 [SMILES: CCCCCCCC(CCCCCCC)OC(=O)CCCN(CCCC(=O)OC(CCCCCCC)CCCCCCC)C(=O)SCCCN(CC)CC]

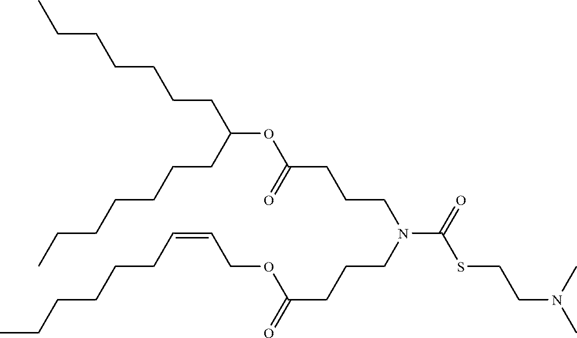 [SMILES: CCCCCC/C=C\COC(=O)CCCN(CCCC(=O)OC(CCCCCCC)CCCCCCC)C(=O)SCCN(C)C]

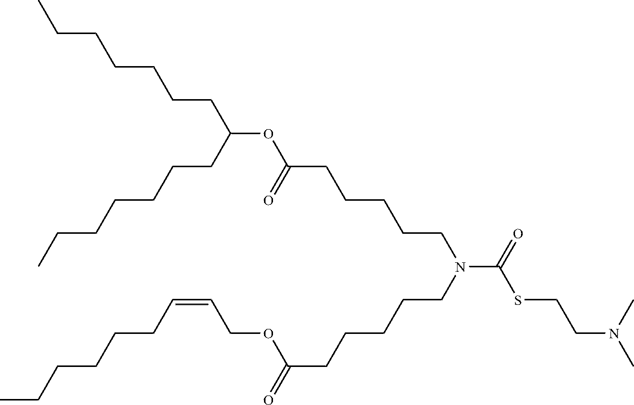 [SMILES: CCCCCC/C=C\COC(=O)CCCCCN(CCCCCC(=O)OC(CCCCCCC)CCCCCCC)C(=O)SCCN(C)C]

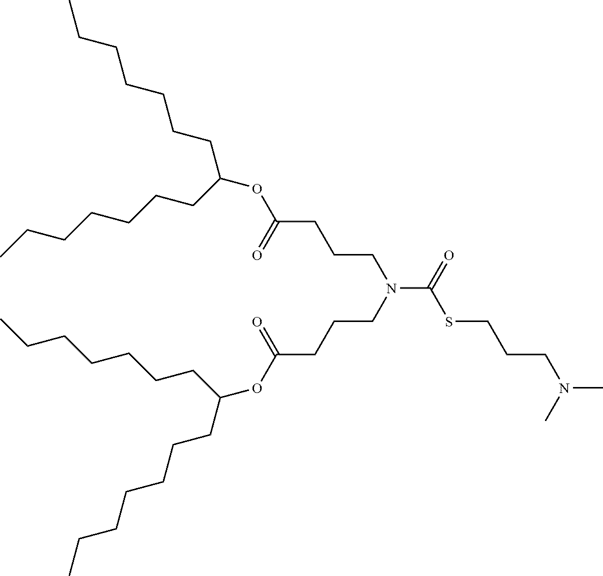 [SMILES: CCCCCCCC(CCCCCCC)OC(=O)CCCN(CCCC(=O)OC(CCCCCCC)CCCCCCC)C(=O)SCCCN(C)C]

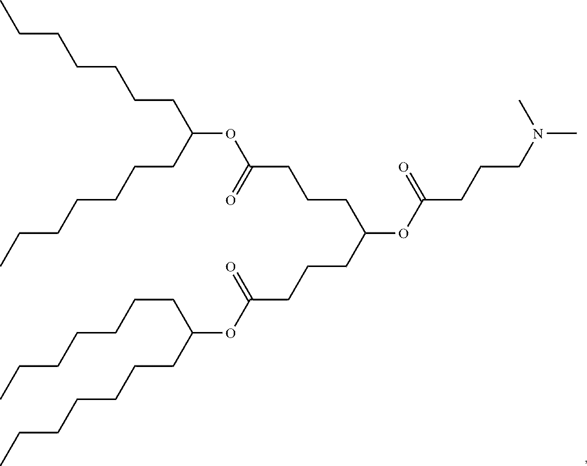

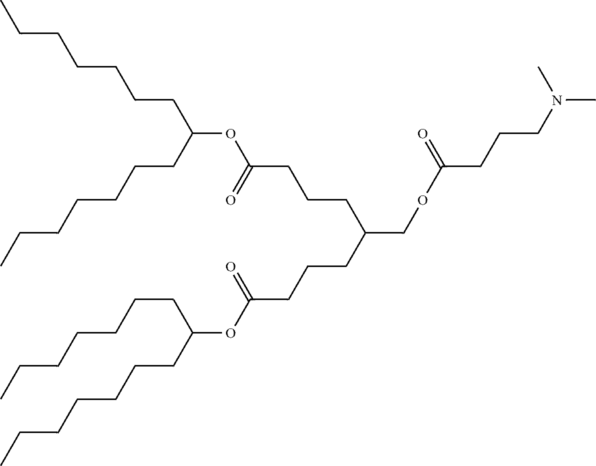 [SMILES: CCCCCCCC(CCCCCCC)OC(=O)CCCC(CCCC(=O)OC(CCCCCCC)CCCCCCC)COC(=O)CCCN(C)C]

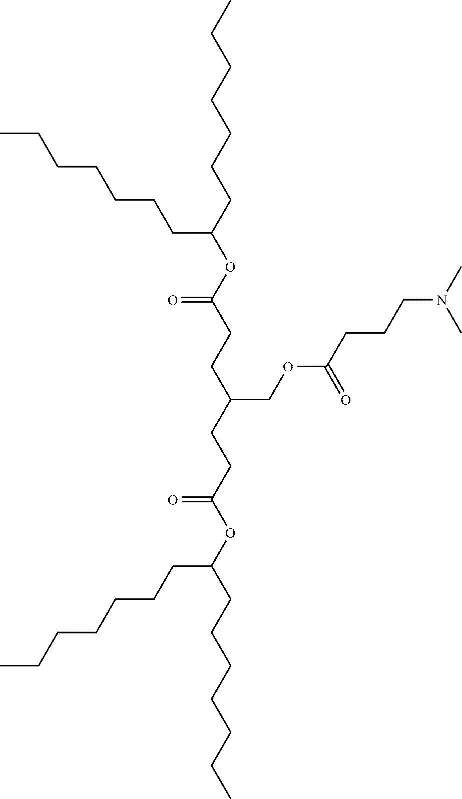 [SMILES: CCCCCCCC(CCCCCCC)OC(=O)CCC(CCC(=O)OC(CCCCCCC)CCCCCCC)COC(=O)CCCN(C)C]

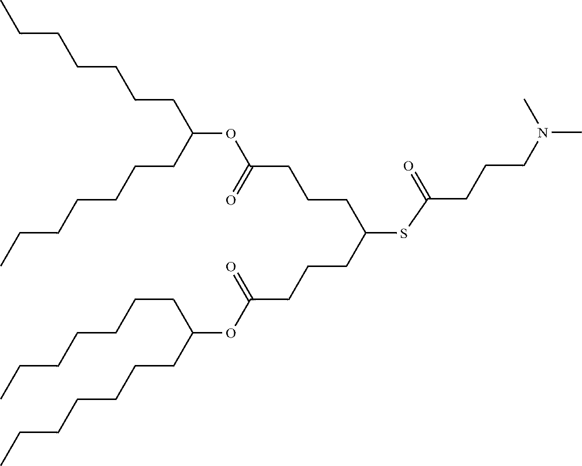 [SMILES: CCCCCCCC(CCCCCCC)OC(=O)CCCC(CCCC(=O)OC(CCCCCCC)CCCCCCC)SC(=O)CCCN(C)C]

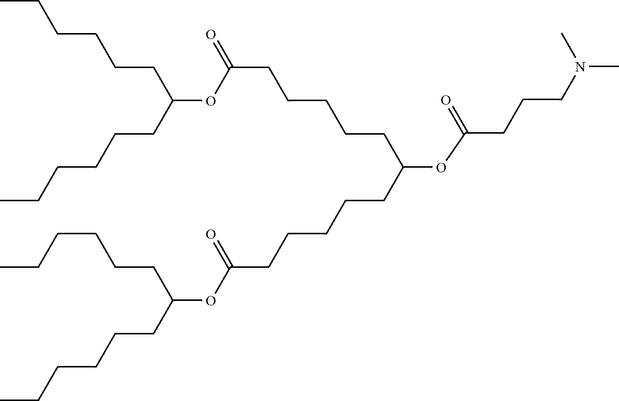 [SMILES: CCCCCCC(CCCCCC)OC(=O)CCCCCC(CCCCCC(=O)OC(CCCCCC)CCCCCC)OC(=O)CCCN(C)C]

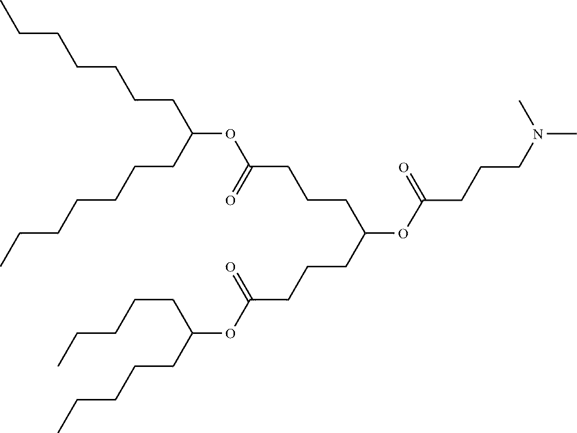 [SMILES: CCCCCCCC(CCCCCCC)OC(=O)CCCC(CCCC(=O)OC(CCCCC)CCCCC)OC(=O)CCCN(C)C]

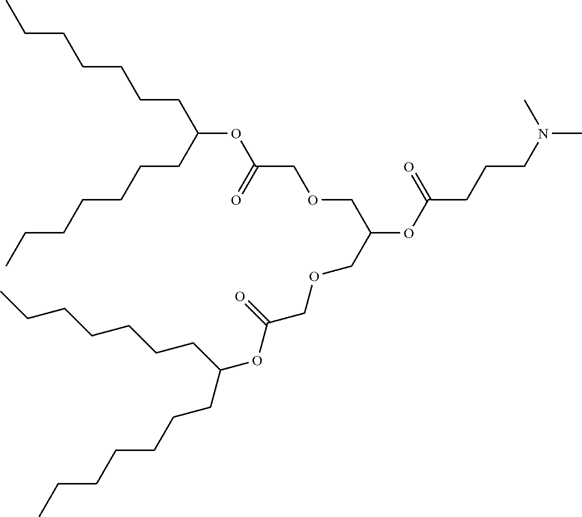 [SMILES: CCCCCCCC(CCCCCCC)OC(=O)COCC(COCC(=O)OC(CCCCCCC)CCCCCCC)OC(=O)CCCN(C)C]

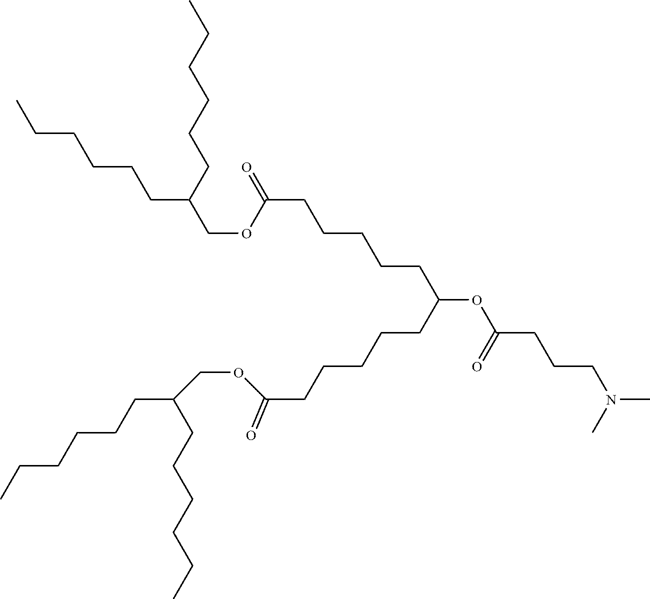 [SMILES: CCCCCCC(CCCCCC)COC(=O)CCCCCC(CCCCCC(=O)OCC(CCCCCC)CCCCCC)OC(=O)CCCN(C)C]

and

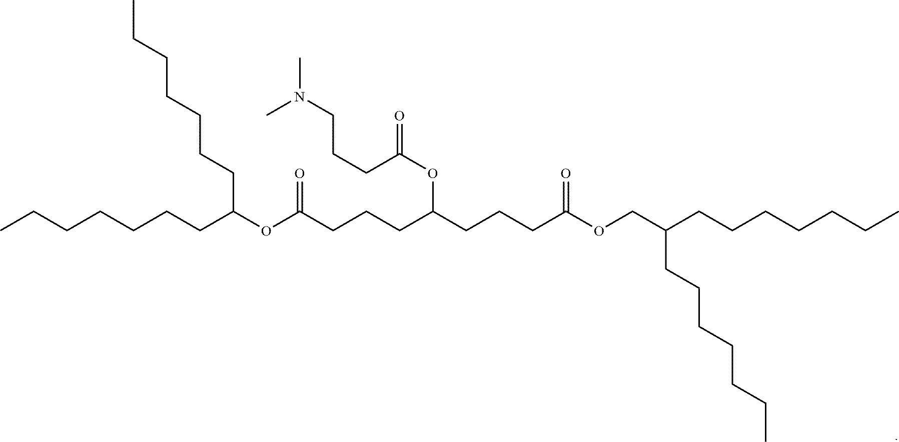

In one aspect, the ionizable cationic lipid of compositions provided herein has a structure of

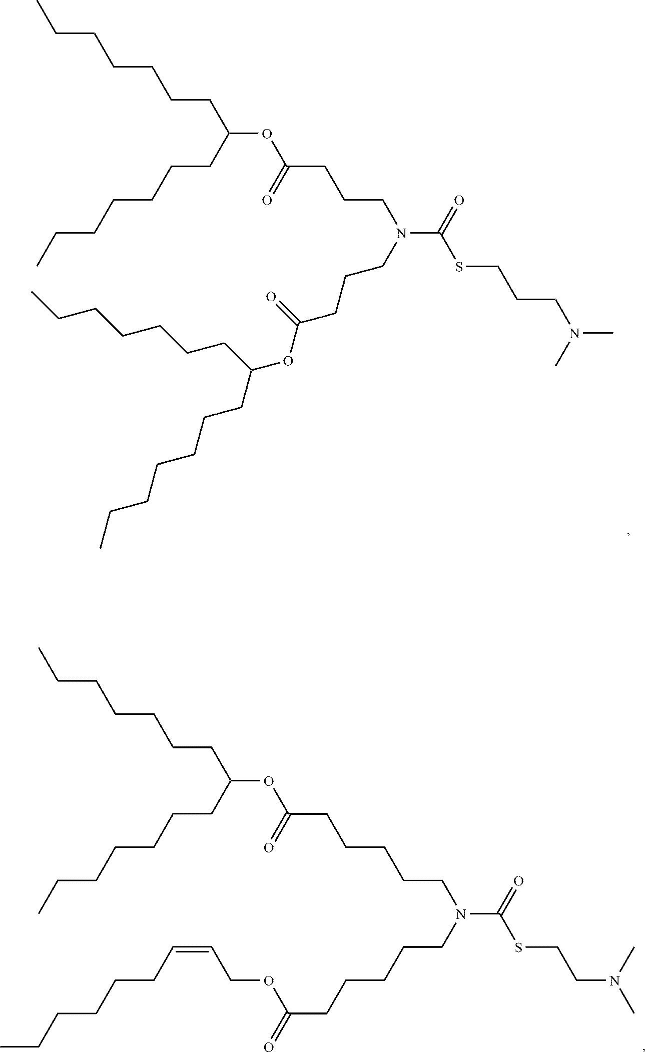

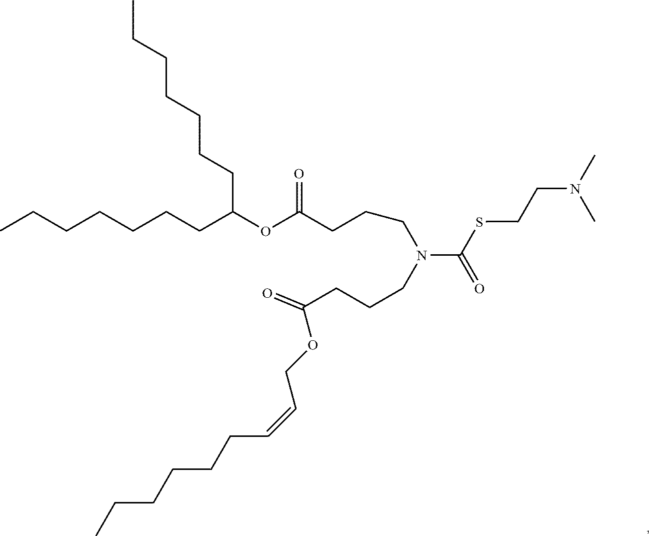

or a pharmaceutically acceptable salt thereof.

In another aspect, the ionizable cationic lipid of compositions provided herein has a structure of

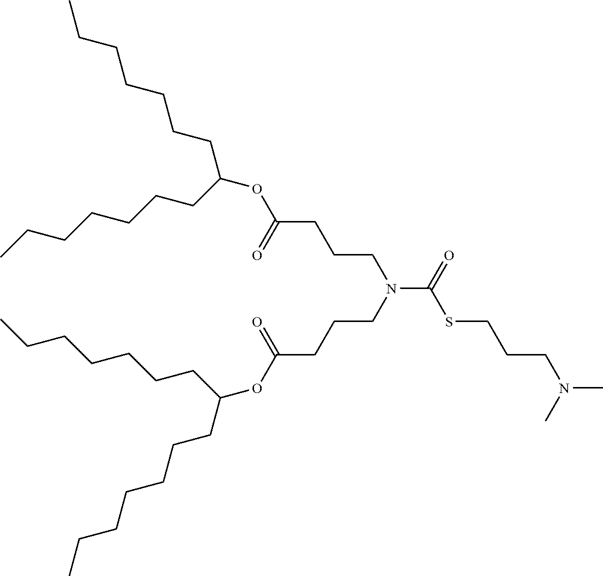 [SMILES: CCCCCCCC(CCCCCCC)OC(=O)CCCN(CCCC(=O)OC(CCCCCCC)CCCCCCC)C(=O)SCCCN(C)C]

or a pharmaceutically acceptable salt thereof.

In one aspect, the ionizable cationic lipid included in lipid formulations of pharmaceutical compositions provided herein has a structure of

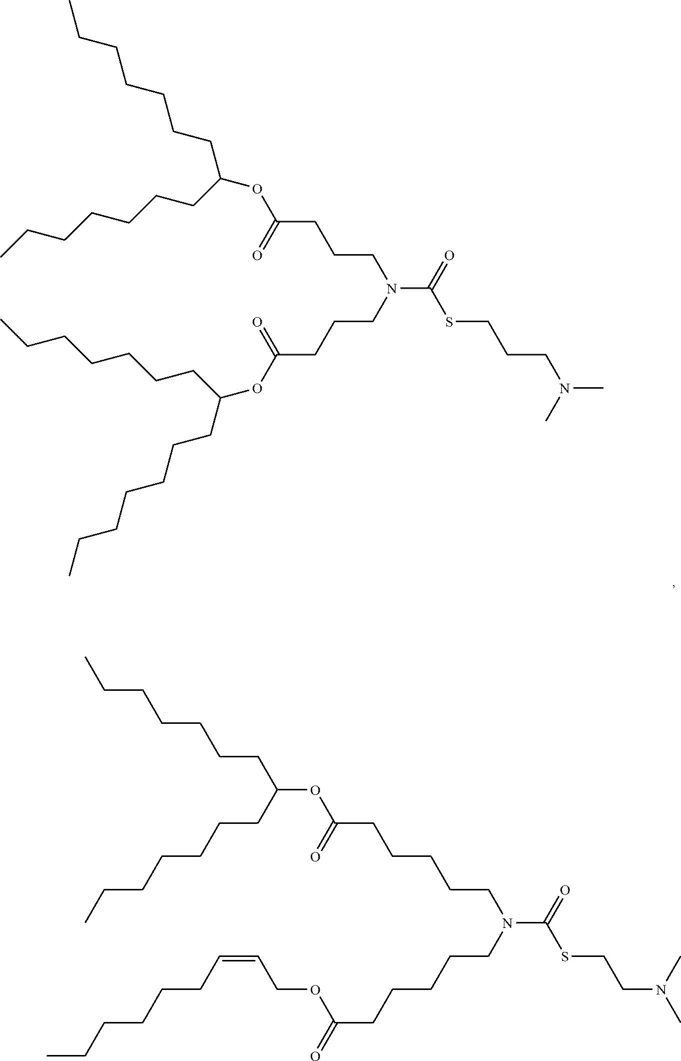

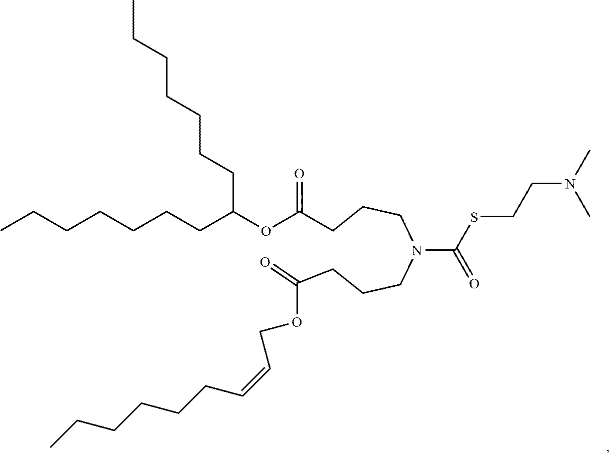

or a pharmaceutically acceptable salt thereof.

In another aspect, the ionizable cationic lipid included in lipid formulations of pharmaceutical compositions provided herein has a structure of

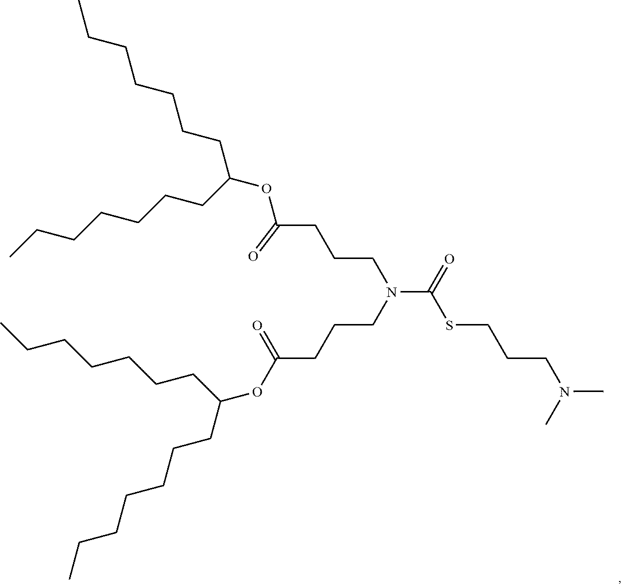

or a pharmaceutically acceptable salt thereof.

### Lipid Formulations/LNPs

Therapies based on the intracellular delivery of nucleic acids to target cells face both extracellular and intracellular barriers. Indeed, naked nucleic acid materials cannot be easily systemically administered due to their toxicity, low stability in serum, rapid renal clearance, reduced uptake by target cells, phagocyte uptake and their ability in activating the immune response, all features that preclude their clinical development. When exogenous nucleic acid material (e.g., mRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida MPharm Res. 1995 Jun; 12(6):825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane (for DNA), release at the cytoplasm (for RNA), and so on. Successful nucleic acid-based therapy thus depends upon the ability of the vector to deliver the nucleic acids to the target sites inside of the cells in order to obtain sufficient levels of a desired activity such as expression of a gene.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the Food and Drug Administration (FDA) and by the European Commission (EC). ALN-TTR02 is an siRNA formulation based upon the so-called Stable Nucleic Acid Lipid Particle (SNALP) transfecting technology. Despite the success of Patisiran, the delivery of nucleic acid therapeutics, including mRNA, via lipid formulations is still under ongoing development.

Some art-recognized lipid-formulated delivery vehicles for nucleic acid therapeutics include, according to various embodiments, polymer based carriers, such as polyethyleneimine (PEI), lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions. These lipid formulations can vary in their structure and composition, and as can be expected in a rapidly evolving field, several different terms have been used in the art to describe a single type of delivery vehicle. At the same time, the terms for lipid formulations have varied as to their intended meaning throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

### Liposomes

Conventional liposomes are vesicles that consist of at least one bilayer and an internal aqueous compartment. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). They generally present as spherical vesicles and can range in size from 20 nm to a few microns. Liposomal formulations can be prepared as a colloidal dispersion or they can be lyophilized to reduce stability risks and to improve the shelf-life for liposome-based drugs. Methods of preparing liposomal compositions are known in the art and would be within the skill of an ordinary artisan.

Liposomes that have only one bilayer are referred to as being unilamellar, and those having more than one bilayer are referred to as multilamellar. The most common types of liposomes are small unilamellar vesicles (SUV), large unilamellar vesicle (LUV), and multilamellar vesicles (MLV). In contrast to liposomes, lysosomes, micelles, and reversed micelles are composed of monolayers of lipids. Generally, a liposome is thought of as having a single interior compartment, however some formulations can be multivesicular liposomes (MVL), which consist of numerous discontinuous internal aqueous compartments separated by several nonconcentric lipid bilayers.

Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9:1833-1843). In their use as drug delivery vehicles, because a liposome has an aqueous solution core surrounded by a hydrophobic membrane, hydrophilic solutes dissolved in the core cannot readily pass through the bilayer, and hydrophobic compounds will associate with the bilayer. Thus, a liposome can be loaded with hydrophobic and/or hydrophilic molecules. When a liposome is used to carry a nucleic acid such as RNA, the nucleic acid will be contained within the liposomal compartment in an aqueous phase.

### Cationic Liposomes

Liposomes can be composed of cationic, anionic, and/or neutral lipids. As an important subclass of liposomes, cationic liposomes are liposomes that are made in whole or part from positively charged lipids, or more specifically a lipid that comprises both a cationic group and a lipophilic portion. In addition to the general characteristics profiled above for liposomes, the positively charged moieties of cationic lipids used in cationic liposomes provide several advantages and some unique structural features. For example, the lipophilic portion of the cationic lipid is hydrophobic and thus will direct itself away from the aqueous interior of the liposome and associate with other nonpolar and hydrophobic species. Conversely, the cationic moiety will associate with aqueous media and more importantly with polar molecules and species with which it can complex in the aqueous interior of the cationic liposome. For these reasons, cationic liposomes are increasingly being researched for use in gene therapy due to their favorability towards negatively charged nucleic acids via electrostatic interactions, resulting in complexes that offer biocompatibility, low toxicity, and the possibility of the large-scale production required for in vivo clinical applications. Cationic lipids suitable for use in cationic liposomes are listed herein below.

### Lipid Nanoparticles

In contrast to liposomes and cationic liposomes, lipid nanoparticles (LNP) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. While sources vary on what size qualifies a lipid particle as being a nanoparticle, there is some overlap in agreement that a lipid nanoparticle can have a diameter in the range of from 10 nm to 1000 nm. However, more commonly they are considered to be smaller than 120 nm or even 100 nm.

For lipid nanoparticle nucleic acid delivery systems, the lipid shell is formulated to include an ionizable cationic lipid which can complex to and associate with the negatively charged backbone of the nucleic acid core. Ionizable cationic lipids with apparent pKa values below about 7 have the benefit of providing a cationic lipid for complexing with the nucleic acid’s negatively charged backbone and loading into the lipid nanoparticle at pH values below the pKa of the ionizable lipid where it is positively charged. Then, at physiological pH values, the lipid nanoparticle can adopt a relatively neutral exterior allowing for a significant increase in the circulation half-lives of the particles following i.v. administration. In the context of nucleic acid delivery, lipid nanoparticles offer many advantages over other lipid-based nucleic acid delivery systems including high nucleic acid encapsulation efficiency, potent transfection, improved penetration into tissues to deliver therapeutics, and low levels of cytotoxicity and immunogenicity.

Prior to the development of lipid nanoparticle delivery systems for nucleic acids, cationic lipids were widely studied as synthetic materials for delivery of nucleic acid medicines. In these early efforts, after mixing together at physiological pH, nucleic acids were condensed by cationic lipids to form lipid-nucleic acid complexes known as lipoplexes. However, lipoplexes proved to be unstable and characterized by broad size distributions ranging from the submicron scale to a few microns. Lipoplexes, such as the Lipofectamine® reagent, have found considerable utility for in vitro transfection. However, these first-generation lipoplexes have not proven useful in vivo. The large particle size and positive charge (Imparted by the cationic lipid) result in rapid plasma clearance, hemolytic and other toxicities, as well as immune system activation.In some aspects, nucleic acid molecules provided herein and lipids or lipid formulations provided herein form a lipid nanoparticle (LNP).

In other aspects, nucleic acid molecules provided herein are incorporated into a lipid formulation (i.e., a lipid-based delivery vehicle).

In the context of the present disclosure, a lipid-based delivery vehicle typically serves to transport a desired RNA to a target cell or tissue. The lipid-based delivery vehicle can be any suitable lipid-based delivery vehicle known in the art. In some aspects, the lipid-based delivery vehicle is a liposome, a cationic liposome, or a lipid nanoparticle containing a self-replicating RNA or mRNA of the disclosure. In some aspects, the lipid-based delivery vehicle comprises a nanoparticle or a bilayer of lipid molecules and a self-replicating RNA or mRNA of the disclosure. In some aspects, the lipid bilayer further comprises a neutral lipid or a polymer. In some aspects, the lipid formulation comprises a liquid medium. In some aspects, the formulation further encapsulates a nucleic acid. In some aspects, the lipid formulation further comprises a nucleic acid and a neutral lipid or a polymer. In some aspects, the lipid formulation encapsulates the nucleic acid.

The description provides lipid formulations comprising one or more RNA molecules encapsulated within the lipid formulation. In some aspects, the lipid formulation comprises liposomes. In some aspects, the lipid formulation comprises cationic liposomes. In some aspects, the lipid formulation comprises lipid nanoparticles.

In some aspects, the self-replicating RNA or mRNA is fully encapsulated within the lipid portion of the lipid formulation such that the RNA in the lipid formulation is resistant in aqueous solution to nuclease degradation. In other aspects, the lipid formulations described herein are substantially non-toxic to animals such as humans and other mammals.

The lipid formulations of the disclosure also typically have a total lipid:RNA ratio (mass/mass ratio) of from about 1:1 to about 100:1, from about 1:1 to about 50:1, from about 2:1 to about 45:1, from about 3:1 to about 40:1, from about 5:1 to about 45:1, or from about 10:1 to about 40:1, or from about 15:1 to about 40:1, or from about 20:1 to about 40:1; or from about 25:1 to about 45:1; or from about 30:1 to about 45:1; or from about 32:1 to about 42:1; or from about 34:1 to about 42:1. In some aspects, the total lipid:RNA ratio (mass/mass ratio) is from about 30:1 to about 45:1. The ratio may be any value or subvalue within the recited ranges, including endpoints.

The lipid formulations of the present disclosure typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm, and are substantially non-toxic. The diameter may be any value or subvalue within the recited ranges, including endpoints. In addition, nucleic acids, when present in the lipid nanoparticles of the present disclosure, generally are resistant in aqueous solution to degradation with a nuclease.

In some embodiments, the lipid nanoparticle has a size of less than about 500 nm, less than about 400 nm, less than about 300 nm, less than about 200 nm, less than about 100 nm, or less than about 50 nm. In specific embodiments, the lipid nanoparticle has a size of about 55 nm to about 90 nm.

In some aspects, the lipid formulations comprise a self-replicating RNA or mRNA, a cationic lipid (e.g., one or more cationic lipids or salts thereof described herein), a phospholipid, and a conjugated lipid that inhibits aggregation of the particles (e.g., one or more PEG-lipid conjugates). The lipid formulations can also include cholesterol. In one aspect, the cationic lipid is an ionizable cationic lipid.

In the nucleic acid-lipid formulations, the RNA may be fully encapsulated within the lipid portion of the formulation, thereby protecting the nucleic acid from nuclease degradation. In some aspects, a lipid formulation comprising an RNA is fully encapsulated within the lipid portion of the lipid formulation, thereby protecting the nucleic acid from nuclease degradation. In certain aspects, the RNA in the lipid formulation is not substantially degraded after exposure of the particle to a nuclease at 37° C. for at least 20, 30, 45, or 60 minutes. In certain other aspects, the RNA in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In some aspects, the RNA is complexed with the lipid portion of the formulation. One of the benefits of the formulations of the present disclosure is that the nucleic acid-lipid compositions are substantially non-toxic to animals such as humans and other mammals.

In the context of nucleic acids, full encapsulation may be determined by performing a membrane-impermeable fluorescent dye exclusion assay, which uses a dye that has enhanced fluorescence when associated with nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E = (I0 - I)/I0, where/and I0 refers to the fluorescence intensities before and after the addition of detergent.

In some aspects, the present disclosure provides a nucleic acid-lipid composition comprising a plurality of nucleic acid-liposomes, nucleic acid-cationic liposomes, or nucleic acid-lipid nanoparticles. In some aspects, the nucleic acid-lipid composition comprises a plurality of RNA-liposomes. In some aspects, the nucleic acid-lipid composition comprises a plurality of RNA-cationic liposomes. In some aspects, the nucleic acid-lipid composition comprises a plurality of RNA-lipid nanoparticles.

In some aspects, the lipid formulations comprise RNA that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the RNA encapsulated therein. The amount may be any value or subvalue within the recited ranges, including endpoints. The RNA included in any RNA-lipid composition or RNA-lipid formulation provided herein can be a self-replicating RNA or an mRNA.

Depending on the intended use of the lipid formulation, the proportions of the components can be varied, and the delivery efficiency of a particular formulation can be measured using assays known in the art.

In some aspects, nucleic acid molecules provided herein are lipid formulated. The lipid formulation is preferably selected from, but not limited to, liposomes, cationic liposomes, and lipid nanoparticles. In one aspect, a lipid formulation is a cationic liposome or a lipid nanoparticle (LNP) comprising:
(a) an RNA of the present disclosure,
(b) a cationic lipid,
(c) an aggregation reducing agent (such as polyethylene glycol (PEG) lipid or PEG-modified lipid),
(d) optionally a non-cationic lipid (such as a neutral lipid), and
(e) optionally, a sterol.

In another aspect, the cationic lipid is an ionizable cationic lipid. Any ionizable cationic lipid can be included in lipid formulations, including exemplary cationic lipids provided herein.

In some aspects, compositions that include lipids and/or lipid formulations provided herein include an RNA molecule comprising (A) a sequence of SEQ ID NO: 1; (B) a sequence of SEQ ID NO:2; (C) a sequence of SEQ ID NO:3; or (D) a sequence of SEQ ID NO:4. In some aspects, compositions provided herein include an RNA molecule comprising a sequence of SEQ ID NO:40. In some aspects, compositions provided herein include an RNA molecule comprising a sequence of SEQ ID NO: 29, SEQ ID NO: 32, or SEQ ID NO:48. In some aspects, compositions provided herein include lipid nanoparticles (LNPs). In some aspects, compositions provided herein include lyophilized LNPs.

Provided herein, in some embodiments, are lipid nanoparticle compositions comprising a. a lipid formulation comprising i. about 45 mol% to about 55 mol% of an ionizable cationic lipid having the structure of ATX-126:

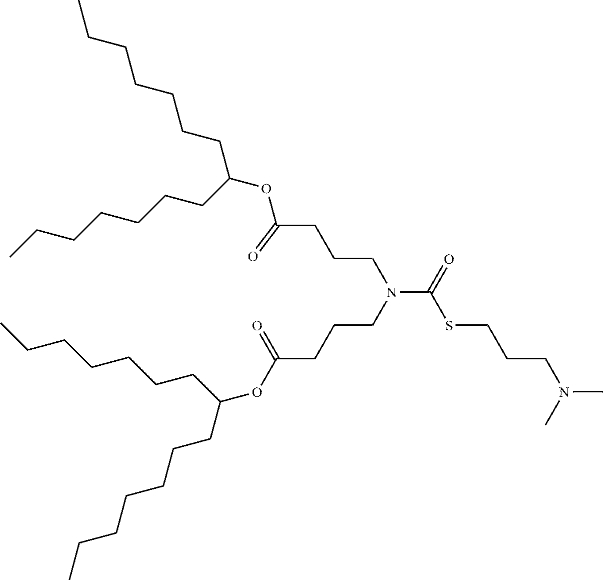 [SMILES: CCCCCCCC(CCCCCCC)OC(=O)CCCN(CCCC(=O)OC(CCCCCCC)CCCCCCC)C(=O)SCCCN(C)C]

ii. about 8 mol% to about 12 mol% DSPC; iii. about 35 mol% to about 42 mol% cholesterol; and iv. about 1.25 mol% to about 1.75 mol% PEG2000-DMG; and b. an RNA molecule having at least 80% identity to a sequence of SEQ ID NO: 1, SEQ ID NO:2, SEQ ID NO:3, or SEQ ID NO:4; wherein the lipid formulation encapsulates the RNA molecule and the lipid nanoparticle has a size of about 60 to about 90 nm. In some aspects, the RNA molecule included in lipid nanoparticle compositions provided herein has at least 80% identity to a sequence of SEQ ID NO:40. In some aspects, the RNA molecule included in lipid nanoparticle compositions provided herein has at least 80% identity to a sequence of SEQ ID NO: 29, SEQ ID NO: 32, or SEQ ID NO:48. In some aspects, lipid nanoparticle compositions provided herein are lyophilized. In some aspects, the RNA molecule included in lipid nanoparticle compositions provided herein has at least 80% identity to a sequence of SEQ ID NO:29. In some aspects, the RNA molecule included in lipid nanoparticle compositions provided herein has at least 80% identity to a sequence of SEQ ID NO: 32.

### Cationic Lipids

In one aspect, the lipid nanoparticle formulation comprises (i) at least one cationic lipid; (ii) a helper lipid; (iii) a sterol (e.g., cholesterol); and (iv) a PEG-lipid. In another aspect, the cationic lipid is an ionizable cationic lipid. In yet another aspect, the lipid nanoparticle formulation comprises (i) at least one cationic lipid; (ii) a helper lipid; (iii) a sterol (e.g., cholesterol); and (iv) a PEG-lipid, in a molar ratio of about 40-70% ionizable cationic lipid: about 2-15% helper lipid: about 20-45% sterol; about 0.5-5% PEG-lipid. In a further aspect, the cationic lipid is an ionizable cationic lipid.

In one aspect, the lipid nanoparticle formulation consists of (i) at least one cationic lipid; (ii) a helper lipid; (iii) a sterol (e.g., cholesterol); and (iv) a PEG-lipid. In another aspect, the cationic lipid is an ionizable cationic lipid. In yet another aspect, the lipid nanoparticle formulation consists of (i) at least one cationic lipid; (ii) a helper lipid; (iii) a sterol (e.g., cholesterol); and (iv) a PEG-lipid, in a molar ratio of about 40-70% ionizable cationic lipid: about 2-15% helper lipid: about 20-45% sterol; about 0.5-5% PEG-lipid. In a further aspect, the cationic lipid is an ionizable cationic lipid.

In the presently disclosed lipid formulations, the cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (y-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N- dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)-N,N -dimethyl-2,2-di( (9Z, 12Z)-octadeca-9, 12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-l-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,3 1-tetraen-19-yloxy)-N,N-dimethylpropan-l-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P-(N-(N′,N′-dimethylaminoethane)- carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Pat. Publication Nos. 2011/0256175, 2012/0128760, and 2012/0027803; U.S. Pat. Nos. 8,158,601; and Love et al., PNAS, 107(5), 1864-69, 2010, the contents of which are herein incorporated by reference.

The RNA-lipid formulations of the present disclosure can comprise a helper lipid, which can be referred to as a neutral helper lipid, non-cationic lipid, non-cationic helper lipid, anionic lipid, anionic helper lipid, or a neutral lipid. It has been found that lipid formulations, particularly cationic liposomes and lipid nanoparticles have increased cellular uptake if helper lipids are present in the formulation. (Curr. Drug Metab. 2014; 15(9):882-92). For example, some studies have indicated that neutral and zwitterionic lipids such as 1,2-dioleoylsn-glycero-3-phosphatidylcholine (DOPC), Di-Oleoyl-Phosphatidyl-Ethanoalamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 Jan; 9(1):105-20). In addition, the use of helper lipids can help to reduce any potential detrimental effects from using many prevalent cationic lipids such as toxicity and immunogenicity.

Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1 -carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl.

Additional examples of non-cationic lipids include sterols such as cholesterol and derivatives thereof. As a helper lipid, cholesterol increases the spacing of the charges of the lipid layer interfacing with the nucleic acid making the charge distribution match that of the nucleic acid more closely. (J. R. Soc. Interface. 2012 Mar 7; 9(68): 548-561). Non-limiting examples of cholesterol derivatives include polar analogues such as 5α-cholestanol, 5α-coprostanol, cholesteryl-(2′-hydroxy)-ethyl ether, cholesteryl-(4′- hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In some aspects, the cholesterol derivative is a polar analogue such as cholesteryl-(4′-hydroxy)-butyl ether.

In some aspects, the helper lipid present in the lipid formulation comprises or consists of a mixture of one or more phospholipids and cholesterol or a derivative thereof. In other aspects, the neutral lipid present in the lipid formulation comprises or consists of one or more phospholipids, e.g., a cholesterol-free lipid formulation. In yet other aspects, the neutral lipid present in the lipid formulation comprises or consists of cholesterol or a derivative thereof, e.g., a phospholipid-free lipid formulation.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

Other suitable cationic lipids include those having alternative fatty acid groups and other dialkylamino groups, including those, in which the alkyl substituents are different (e.g., N-ethyl- N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid.

In some embodiments, the lipid formulation comprises the cationic lipid with Formula I according to the patent application PCT/EP2017/064066. In this context, the disclosure of PCT/EP2017/064066 is also incorporated herein by reference.

In some embodiments, amino or cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. Of course, it will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

In some embodiments, the lipid formulation comprises an ionizable cationic lipid of Formula I:

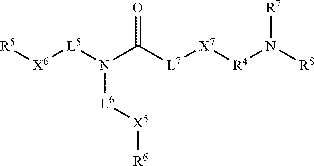

or a pharmaceutically acceptable salt or solvate thereof, wherein R5 and R6 are each independently selected from the group consisting of a linear or branched C1-C31 alkyl, C2-C31 alkenyl or C2-C31 alkynyl and cholesteryl; L5 and L6 are each independently selected from the group consisting of a linear C1-C20 alkyl and C2-C20 alkenyl; X5 is —C(O)O—, whereby —C(O)O—R6 is formed or —OC(O)— whereby —OC(O)—R6 is formed; X6 is —C(O)O—whereby —C(O)O—R5 is formed or —OC(O)— whereby —OC(O)—R5 is formed; X7 is S or O; L7 is absent or lower alkyl; R4 is a linear or branched C1-C6 alkyl; and R7 and R8 are each independently selected from the group consisting of a hydrogen and a linear or branched C1-C6 alkyl.

In some embodiments, X7 is S.

In some embodiments, X5 is —C(O)O—, whereby —C(O)O—R6 is formed and X6 is —C(O)O— whereby —C(O)O—R5 is formed.

In some embodiments, R7 and R8 are each independently selected from the group consisting of methyl, ethyl and isopropyl.

In some embodiments, L5 and L6 are each independently a C1-C10 alkyl. In some embodiments, L5 is C1-C3 alkyl, and L6 is C1-C5 alkyl. In some embodiments, L6 is C1-C2 alkyl. In some embodiments, L5 and L6 are each a linear C7 alkyl. In some embodiments, L5 and L6 are each a linear C9 alkyl.

In some embodiments, R5 and R6 are each independently an alkenyl. In some embodiments, R6 is alkenyl. In some embodiments, R6 is C2-C9 alkenyl. In some embodiments, the alkenyl comprises a single double bond. In some embodiments, R5 and R6 are each alkyl. In some embodiments, R5 is a branched alkyl. In some embodiments, R5 and R6 are each independently selected from the group consisting of a C9 alkyl, C9 alkenyl and C9 alkynyl. In some embodiments, R5 and R6 are each independently selected from the group consisting of a C11 alkyl, C11 alkenyl and C11 alkynyl. In some embodiments, R5 and R6 are each independently selected from the group consisting of a C7 alkyl, C7 alkenyl and C7 alkynyl. In some embodiments, R5 is —CH((CH2)pCH3)2 or —CH((CH2)pCH3)((CH2)p—1CH3), wherein p is 4-8. In some embodiments, p is 5 and L5 is a C1-C3 alkyl. In some embodiments, p is 6 and L5 is a C3 alkyl. In some embodiments, p is 7. In some embodiments, p is 8 and L5 is a C1-C3 alkyl. In some embodiments, R5 consists of —CH((CH2)pCH3)((CH2)p—1CH3), wherein p is 7 or 8.

In some embodiments, R4 is ethylene or propylene. In some embodiments, R4 is n-propylene or isobutylene.

In some embodiments, L7 is absent, R4 is ethylene, X7 is S and R7 and R8 are each methyl. In some embodiments, L7 is absent, R4 is n-propylene, X7 is S and R7 and R8 are each methyl. In some embodiments, L7 is absent, R4 is ethylene, X7 is S and R7 and R8 are each ethyl.

In some embodiments, X7 is S, X5 is —C(O)O—, whereby —C(O)O—R6 is formed, X6 is —C(O)O— whereby —C(O)O—R5 is formed, L5 and L6 are each independently a linear C3-C7 alkyl, L7 is absent, R5 is —CH((CH2)pCH3)2, and R6 is C7-C12 alkenyl. In some further embodiments, p is 6 and R6 is C9 alkenyl.

In embodiments, any one or more lipids recited herein may be expressly excluded.

In some aspects, the helper lipid comprises from about 2 mol% to about 20 mol%, from about 3 mol% to about 18 mol%, from about 4 mol% to about 16 mol%, about 5 mol% to about 14 mol%, from about 6 mol% to about 12 mol%, from about 5 mol% to about 10 mol%, from about 5 mol% to about 9 mol%, or about 2 mol%, about 3 mol%, about 4 mol%, about 5 mol%, about 6 mol%, about 7 mol%, about 8 mol%, about 9 mol%, about 10 mol%, about 11 mol%, or about 12 mol% (or any fraction thereof or the range therein) of the total lipid present in the lipid formulation.

The lipid portion, or the cholesterol or cholesterol derivative in the lipid formulation may comprise up to about 40 mol%, about 45 mol%, about 50 mol%, about 55 mol%, or about 60 mol% of the total lipid present in the lipid formulation. In some aspects, the cholesterol or cholesterol derivative comprises about 15 mol% to about 45 mol%, about 20 mol% to about 40 mol%, about 25 mol% to about 35 mol%, or about 28 mol% to about 35 mol%; or about 25 mol%, about 26 mol%, about 27 mol%, about 28 mol%, about 29 mol%, about 30 mol%, about 31 mol%, about 32 mol%, about 33 mol%, about 34 mol%, about 35 mol%, about 36 mol%, or about 37 mol% of the total lipid present in the lipid formulation.

In specific embodiments, the lipid portion of the lipid formulation is about 35 mol% to about 42 mol% cholesterol.

In some aspects, the phospholipid component in the mixture may comprise from about 2 mol% to about 20 mol%, from about 3 mol% to about 18 mol%, from about 4 mol % to about 16 mol %, about 5 mol % to about 14 mol %, from about 6 mol % to about 12 mol%, from about 5 mol% to about 10 mol%, from about 5 mol% to about 9 mol%, or about 2 mol%, about 3 mol%, about 4 mol%, about 5 mol%, about 6 mol%, about 7 mol%, about 8 mol%, about 9 mol%, about 10 mol%, about 11 mol%, or about 12 mol% (or any fraction thereof or the range therein) of the total lipid present in the lipid formulation.

In certain embodiments, the lipid portion of the lipid formulation comprises about, but is not necessarily limited to, 40 mol% to about 60 mol% of the ionizable cationic lipid, about 4 mol% to about 16 mol% DSPC, about 30 mol% to about 47 mol% cholesterol, and about 0.5 mol% to about 3 mol% PEG2000-DMG.

In certain embodiments, the lipid portion of the lipid formulation may comprise, but is not necessarily limited to, about 42 mol% to about 58 mol% of the ionizable cationic lipid, about 6 mol% to about 14 mol% DSPC, about 32 mol% to about 44 mol% cholesterol, and about 1 mol% to about 2 mol% PEG2000-DMG.

In certain embodiments, the lipid portion of the lipid formulation may comprise, but is not necessarily limited to, about 45 mol% to about 55 mol% of the ionizable cationic lipid, about 8 mol% to about 12 mol% DSPC, about 35 mol% to about 42 mol% cholesterol, and about 1.25 mol% to about 1.75 mol% PEG2000-DMG.

The percentage of helper lipid present in the lipid formulation is a target amount, and the actual amount of helper lipid present in the formulation may vary, for example, by ± 5 mol%.

A lipid formulation that includes a cationic lipid compound or ionizable cationic lipid compound may be on a molar basis about 30-70% cationic lipid compound, about 25-40 % cholesterol, about 2-15% helper lipid, and about 0.5-5% of a polyethylene glycol (PEG) lipid, wherein the percent is of the total lipid present in the formulation. In some aspects, the composition is about 40-65% cationic lipid compound, about 25- 35% cholesterol, about 3-9% helper lipid, and about 0.5-3% of a PEG-lipid, wherein the percent is of the total lipid present in the formulation.

The formulation may be a lipid particle formulation, for example containing 8-30% nucleic acid compound, 5-30% helper lipid, and 0-20% cholesterol; 4-25% cationic lipid, 4-25% helper lipid, 2- 25% cholesterol, 10- 35% cholesterol-PEG, and 5% cholesterol-amine; or 2-30% cationic lipid, 2-30% helper lipid, 1-15% cholesterol, 2-35% cholesterol-PEG, and 1-20% cholesterol-amine; or up to 90% cationic lipid and 2-10% helper lipids, or even 100% cationic lipid.

### Lipid Conjugates

The lipid formulations described herein may further comprise a lipid conjugate. The conjugated lipid is useful in that it prevents the aggregation of particles. Suitable conjugated lipids include, but are not limited to, PEG-lipid conjugates, cationic-polymer-lipid conjugates, and mixtures thereof. Furthermore, lipid delivery vehicles can be used for specific targeting by attaching ligands (e.g., antibodies, peptides, and carbohydrates) to its surface or to the terminal end of the attached PEG chains (Front Pharmacol. 2015 Dec 1; 6:286).

In some aspects, the lipid conjugate is a PEG-lipid. The inclusion of polyethylene glycol (PEG) in a lipid formulation as a coating or surface ligand, a technique referred to as PEGylation, helps to protect nanoparticles from the immune system and their escape from RES uptake (Nanomedicine (Lond). 2011 Jun; 6(4):715-28). PEGylation has been used to stabilize lipid formulations and their payloads through physical, chemical, and biological mechanisms. Detergent-like PEG lipids (e.g., PEG-DSPE) can enter the lipid formulation to form a hydrated layer and steric barrier on the surface. Based on the degree of PEGylation, the surface layer can be generally divided into two types, brush-like and mushroom-like layers. For PEG-DSPE-stabilized formulations, PEG will take on the mushroom conformation at a low degree of PEGylation (usually less than 5 mol%) and will shift to brush conformation as the content of PEG-DSPE is increased past a certain level (Journal of Nanomaterials. 2011;2011:12). PEGylation leads to a significant increase in the circulation half-life of lipid formulations (Annu. Rev. Biomed. Eng. 2011 Aug 15; 13():507-30; J. Control Release. 2010 Aug 3; 145(3):178-81).

Examples of PEG-lipids include, but are not limited to, PEG coupled to dialkyloxypropyls (PEG-DAA), PEG coupled to diacylglycerol (PEG-DAG), methoxypolyethyleneglycol (PEG-DMG or PEG2000-DMG), PEG coupled to phospholipids such as phosphatidylethanolamine (PEG-PE), PEG conjugated to ceramides, PEG conjugated to cholesterol or a derivative thereof, and mixtures thereof.

PEG is a linear, water-soluble polymer of ethylene PEG repeating units with two terminal hydroxyl groups. PEGs are classified by their molecular weights and include the following: monomethoxypolyethylene glycol (MePEG-OH), monomethoxypolyethylene glycol- succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), monomethoxypolyethylene glycol-imidazolyl-carbonyl (MePEG-IM), as well as such compounds containing a terminal hydroxyl group instead of a terminal methoxy group (e.g., HO-PEG-S, HO-PEG-S-NHS, HO-PEG-NH2).

The PEG moiety of the PEG-lipid conjugates described herein may comprise an average molecular weight ranging from about 550 daltons to about 10,000 daltons. In certain aspects, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons). In some aspects, the PEG moiety has an average molecular weight of about 2,000 daltons or about 750 daltons. The average molecular weight may be any value or subvalue within the recited ranges, including endpoints.

In certain aspects, the PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester-containing linker moieties and ester-containing linker moieties. In one aspect, the linker moiety is a non-ester-containing linker moiety. Exemplary non-ester-containing linker moieties include, but are not limited to, amido (—C(O)NH—), amino (—NR—), carbonyl (—C(O)—), carbamate (—NHC(O)O—), urea (—NHC(O)NH, disulfide (—S—S, ether (—O—, succinyl (—(O)CCH2CH2C(O)—), succinamidyl (—NHC(O)CH2CH2C(O)NH—), ether, as well as combinations thereof (such as a linker containing both a carbamate linker moiety and an amido linker moiety). In one aspect, a carbamate linker is used to couple the PEG to the lipid.

In some aspects, an ester-containing linker moiety is used to couple the PEG to the lipid. Exemplary ester-containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the lipid conjugate. Such phosphatidylethanolamines are commercially available or can be isolated or synthesized using conventional techniques known to those of skill in the art. Phosphatidylethanolamines containing saturated or unsaturated fatty acids with carbon chain lengths in the range of C₁₀ to C₂₀ are preferred. Phosphatidylethanolamines with mono- or di-unsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoylphosphatidylethanolamine (DOPE), and distearoyl-phosphatidylethanolamine (DSPE).

In some aspects, the PEG-DAA conjugate is a PEG-didecyloxypropyl (C10) conjugate, a PEG-dilauryloxypropyl (C12) conjugate, a PEG-dimyristyloxypropyl (C14) conjugate, a PEG-dipalmityloxypropyl (C16) conjugate, or a PEG-distearyloxypropyl (C18) conjugate. In some aspects, the PEG has an average molecular weight of about 750 or about 2,000 daltons. In some aspects, the terminal hydroxyl group of the PEG is substituted with a methyl group.

In addition to the foregoing, other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl, methacrylamide, polymethacrylamide, and polydimethylacrylamide, polylactic acid, polyglycolic acid, and derivatized celluloses such as hydroxymethylcellulose or hydroxyethylcellulose.

In some aspects, the lipid conjugate (e.g., PEG-lipid) comprises from about 0.1 mol% to about 2 mol%, from about 0.5 mol% to about 2 mol%, from about 1 mol% to about 2 mol%, from about 0.6 mol% to about 1.9 mol%, from about 0.7 mol% to about 1.8 mol%, from about 0.8 mol% to about 1.7 mol%, from about 0.9 mol% to about 1.6 mol%, from about 0.9 mol% to about 1.8 mol%, from about 1 mol% to about 1.8 mol%, from about 1 mol% to about 1.7 mol%, from about 1.2 mol% to about 1.8 mol%, from about 1.2 mol% to about 1.7 mol%, from about 1.3 mol% to about 1.6 mol%, or from about 1.4 mol% to about 1.6 mol% (or any fraction thereof or range therein) of the total lipid present in the lipid formulation. In other embodiments, the lipid conjugate (e.g., PEG-lipid) comprises about 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, or 5%, (or any fraction thereof or range therein) of the total lipid present in the lipid formulation. The amount may be any value or subvalue within the recited ranges, including endpoints.

The percentage of lipid conjugate (e.g., PEG-lipid) present in the lipid formulations of the disclosure is a target amount, and the actual amount of lipid conjugate present in the formulation may vary, for example, by ± 0.5 mol%. One of ordinary skill in the art will appreciate that the concentration of the lipid conjugate can be varied depending on the lipid conjugate employed and the rate at which the lipid formulation is to become fusogenic.

In some embodiments, the lipid formulation for any of the compositions described herein comprises a lipoplex, a liposome, a lipid nanoparticle, a polymer-based particle, an exosome, a lamellar body, a micelle, or an emulsion.

### Mechanism of Action for Cellular Uptake of Lipid Formulations

In some aspects, lipid formulations for the intracellular delivery of nucleic acids, particularly liposomes, cationic liposomes, and lipid nanoparticles, are designed for cellular uptake by penetrating target cells through exploitation of the target cells′ endocytic mechanisms where the contents of the lipid delivery vehicle are delivered to the cytosol of the target cell. (Nucleic Acid Therapeutics, 28(3): 146-157, 2018). Prior to endocytosis, functionalized ligands such as PEG-lipid at the surface of the lipid delivery vehicle are shed from the surface, which triggers internalization into the target cell. During endocytosis, some part of the plasma membrane of the cell surrounds the vector and engulfs it into a vesicle that then pinches off from the cell membrane, enters the cytosol and ultimately enters and moves through the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For RNA payloads, the cell’s own internal translation processes will then translate the RNA into the encoded protein. The encoded protein can further undergo postranslational processing, including transportation to a targeted organelle or location within the cell or excretion from the cell.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

### Lipid Formulation Manufacture

There are many different methods for the preparation of lipid formulations comprising a nucleic acid. (Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual assymetric centrifugation, ethanol injection, detergent dialysis, spontaneous vesicle formation by ethanol dilution, and encapsulation in preformed liposomes are briefly described herein.

### Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

### Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

### Reverse Phase Evaporation

The Reverse Phase Evaporation (REV) method also allows one to achieve LUVs loaded with nucleic acid. In this technique a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

### Microfluidic Preparation

The Microfluidic method, unlike other bulk techniques, gives the possibility of controlling the lipid hydration process. The method can be classified in continuous-flow microfluidic and droplet-based microfluidic, according to the way in which the flow is manipulated. In the microfluidic hydrodynamic focusing (MHF) method, which operates in a continuous flow mode, lipids are dissolved in isopropyl alcohol which is hydrodynamically focused in a microchannel cross junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

### Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

### Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

### Detergent Dialysis

The Detergent dialysis method can be used to encapsulate nucleic acids. Briefly lipid and plasmid are solubilized in a detergent solution of appropriate ionic strength, after removing the detergent by dialysis, a stabilized lipid formulation is formed. Unencapsulated nucleic acid is then removed by ion-exchange chromatography and empty vesicles by sucrose density gradient centrifugation. The technique is highly sensitive to the cationic lipid content and to the salt concentration of the dialysis buffer, and the method is also difficult to scale.

### Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

### Encapsulation in Preformed Liposomes

The entrapment of nucleic acids can also be obtained starting with preformed liposomes through two different methods: (1) A simple mixing of cationic liposomes with nucleic acids which gives electrostatic complexes called “lipoplexes”, where they can be successfully used to transfect cell cultures, but are characterized by their low encapsulation efficiency and poor performance in vivo; and (2) a liposomal destabilization, slowly adding absolute ethanol to a suspension of cationic vesicles up to a concentration of 40% v/v followed by the dropwise addition of nucleic acids achieving loaded vesicles; however, the two main steps characterizing the encapsulation process are too sensitive, and the particles have to be downsized.

### Excipients

The pharmaceutical compositions disclosed herein can be formulated using one or more excipients to: (1) increase stability; (2) increase cell transfection; (3) permit a sustained or delayed release (e.g., from a depot formulation of the polynucleotide, primary construct, or RNA); (4) alter the biodistribution (e.g., target the polynucleotide, primary construct, or RNA to specific tissues or cell types); (5) increase the translation of encoded protein in vivo; and/or (6) alter the release profile of encoded protein in vivo.

The pharmaceutical compositions described herein may be prepared by any method known or hereafter developed in the art of pharmacology. In general, such preparatory methods include the step of associating the active ingredient (i.e., nucleic acid) with an excipient and/or one or more other accessory ingredients. A pharmaceutical composition in accordance with the present disclosure may be prepared, packaged, and/or sold in bulk, as a single unit dose, and/or as a plurality of single unit doses.

Pharmaceutical compositions may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, liposomes, lipid nanoparticles, polymers, lipoplexes, core-shell nanoparticles, peptides, proteins, cells transfected with primary DNA construct, or RNA (e.g., for transplantation into a subject), hyaluronidase, nanoparticle mimics and combinations thereof.

Accordingly, the pharmaceutical compositions described herein can include one or more excipients, each in an amount that together increases the stability of the nucleic acid in the lipid formulation, increases cell transfection by the nucleic acid, increases the expression of the encoded protein, and/or alters the release profile of encoded proteins. Further, the RNA of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the embodiments of the present disclosure, except insofar as any conventional excipient medium may be incompatible with a substance or its derivatives, such as by producing any undesirable biological effect or otherwise interacting in a deleterious manner with any other component(s) of the pharmaceutical composition.

The pharmaceutical compositions of this disclosure may further contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the RNA-lipid formulation may be administered in a time release formulation, for example in a composition which includes a slow release polymer. The active agent can be prepared with carriers that will protect against rapid release, for example a controlled release vehicle such as a polymer, microencapsulated delivery system, or a bioadhesive gel. Prolonged delivery of the RNA, in various compositions of the disclosure can be brought about by including in the composition agents that delay absorption, for example, aluminum monostearate hydrogels and gelatin.

### Methods of Inducing Immune Responses

Provided herein, in some embodiments, are methods of inducing an immune response in a subj ect. Any type of immune response can be induced using the methods provided herein, including adaptive and innate immune responses. In one aspect, immune responses induced using the methods provided herein include an antibody response, a cellular immune response, or both an antibody response and a cellular immune response.

Methods of inducing an immune response provided herein include administering to a subject an effective amount of any RNA or DNA molecule, i.e., nucleic acid molecule, provided herein. In one aspect, methods of inducing an immune response include administering to a subject an effective amount of any composition comprising an RNA molecule and a lipid provided herein. In another aspect, methods of inducing an immune response include administering to a subject an effective amount of any pharmaceutical composition comprising an RNA molecule and a lipid formulation provided herein. In some aspects, RNA molecules, compositions, and pharmaceutical composition provided here are vaccines that can elicit a protective or a therapeutic immune response, for example.

As used herein, the term “subject” refers to any individual or patient on which the methods disclosed herein are performed. The term “subject” can be used interchangeably with the term “individual” or “patient.” The subject can be a human, although the subject may be an animal, as will be appreciated by those in the art. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject. As used herein, the term “effective amount” or “therapeutically effective amount” refers to that amount of an RNA molecule, composition, or pharmaceutical composition described herein that is sufficient to effect the intended application, including but not limited to inducing an immune response and/or disease treatment, as defined herein. The therapeutically effective amount may vary depending upon the intended application (e.g., inducing an immune response, treatment, application in vivo), or the subject or patient and disease condition being treated, e.g., the weight and age of the subject, the species, the severity of the disease condition, the manner of administration and the like, which can readily be determined by one of ordinary skill in the art. The term also applies to a dose that will induce a particular response in a target cell. The specific dose will vary depending on the particular RNA molecule, composition, or pharmaceutical composition chosen, the dosing regimen to be followed, whether it is administered in combination with other compounds, timing of administration, the tissue to which it is administered, and the physical delivery system in which it is carried.

Exemplary doses of nucleic molecules that can be administered include about 0.01 µg, about 0.02 µg, about 0.03 µg, about 0.04 µg, about 0.05 µg, about 0.06 µg, about 0.07 µg, about 0.08 µg, about 0.09 µg, about 0.1 µg, about 0.2 µg, about 0.3 µg, about 0.4 µg, about 0.5 µg, about 0.6 µg, about 0.7 µg, about 0.8 µg, about 0.9 µg, about 1.0 µg, about 1.5 µg, about 2.0 µg, about 2.5 µg, about 3.0 µg, about 3.5 µg, about 4.0 µg, about 4.5 µg, about 5.0 µg, about 5.5 µg, about 6.0 µg, about 6.5 µg, about 7.0 µg, about 7.5 µg, about 8.0 µg, about 8.5 µg, about 9.0 µg, about 9.5 µg, about 10 µg, about 11 µg, about 12 µg, about 13 µ, about 14 µg, about 15 µg, about 16 µg, about 17 µg, about 18 µg, about 19 µg, about 20 µg, about 21 µg, about 22 µg, about 23 µg, about 24 µg, about 25 µg, about 26 µg, about 27 µg, about 28 µg, about 29 µg, about 30 µg, about 35 µg, about 40 µg, about 45 µg, about 50 µg, about 55 µg, about 60 µg, about 65 µg, about 70 µg, about 75 µg, about 80 µg, about 85 µg, about 90 µg, about 95 µg, about 100 µg, about 125 µg, about 150 µg, about 175 µg, about 200 µg, about 250 µg, about 300 µg, about 350 µg, about 400 µg, about 450 µg, about 500 µg, about 600 µg, about 700 µg, about 800 µg, about 900 µg, about 1,000 µg, or more, and any number or range in between. In one aspect, the nucleic acid molecules are RNA molecules. In another aspect, the nucleic acid molecules are DNA molecules. Nucleic acid molecules can have a unit dosage comprising about 0.01 µg to about 1,000 µg or more nucleic acid in a single dose.

In some aspects, compositions provided herein that can be administered include about 0.01 µg, about 0.02 µg, about 0.03 µg, about 0.04 µg, about 0.05 µg, about 0.06 µg, about 0.07 µg, about 0.08 µg, about 0.09 µg, about 0.1 µg, about 0.2 µg, about 0.3 µg, about 0.4 µg, about 0.5 µg, about 0.6 µg, about 0.7 µg, about 0.8 µg, about 0.9 µg, about 1.0 µg, about 1.5 µg, about 2.0 µg, about 2.5 µg, about 3.0 µg, about 3.5 µg, about 4.0 µg, about 4.5 µg, about 5.0 µg, about 5.5 µg, about 6.0 µg, about 6.5 µg, about 7.0 µg, about 7.5 µg, about 8.0 µg, about 8.5 µg, about 9.0 µg, about 9.5 µg, about 10 µg, about 11 µg, about 12 µg, about 13 µg, about 14 µg, about 15 µg, about 16 µg, about 17 µg, about 18 µg, about 19 µg, about 20 µg, about 21 µg, about 22 µg, about 23 µg, about 24 µg, about 25 µg, about 26 µg, about 27 µg, about 28 µg, about 29 µg, about 30 µg, about 35 µg, about 40 µg, about 45 µg, about 50 µg, about 55 µg, about 60 µg, about 65 µg, about 70 µg, about 75 µg, about 80 µg, about 85 µg, about 90 µg, about 95 µg, about 100 µg, about 125 µg, about 150 µg, about 175 µg, about 200 µg, about 250 µg, about 300 µg, about 350 µg, about 400 µg, about 450 µg, about 500 µg, about 600 µg, about 700 µg, about 800 µg, about 900 µg, about 1,000 µg, or more, and any number or range in between, nucleic acid and lipid. In other aspects, pharmaceutical compositions provided herein that can be administered include about 0.01 µg, about 0.02 µg, about 0.03 µg, about 0.04 µg, about 0.05 µg, about 0.06 µg, about 0.07 µg, about 0.08 µg, about 0.09 µg, about 0.1 µg, about 0.2 µg, about 0.3 µg, about 0.4 µg, about 0.5 µg, about 0.6 µg, about 0.7 µg, about 0.8 µg, about 0.9 µg, about 1.0 µg, about 1.5 µg, about 2.0 µg, about 2.5 µg, about 3.0 µg, about 3.5 µg, about 4.0 µg, about 4.5 µg, about 5.0 µg, about 5.5 µg, about 6.0 µg, about 6.5 µg, about 7.0 µg, about 7.5 µg, about 8.0 µg, about 8.5 µg, about 9.0 µg, about 9.5 µg, about 10 µg, about 11 µg, about 12 µg, about 13 µg, about 14 µg, about 15 µg, about 16 µg, about 17 µg, about 18 µg, about 19 µg, about 20 µg, about 21 µg, about 22 µg, about 23 µg, about 24 µg, about 25 µg, about 26 µg, about 27 µg, about 28 µg, about 29 µg, about 30 µg, about 35 µg, about 40 µg, about 45 µg, about 50 µg, about 55 µg, about 60 µg, about 65 µg, about 70 µg, about 75 µg, about 80 µg, about 85 µg, about 90 µg, about 95 µg, about 100 µg, about 125 µg, about 150 µg, about 175 µg, about 200 µg, about 250 µg, about 300 µg, about 350 µg, about 400 µg, about 450 µg, about 500 µg, about 600 µg, about 700 µg, about 800 µg, about 900 µg, about 1,000 µg, or more, and any number or range in between, nucleic acid and lipid formulation.

In one aspect, compositions provided herein can have a unit dosage comprising about 0.01 µg to about 1,000 µg or more nucleic acid and lipid in a single dose. In another aspect, pharmaceutical compositions provided herein can have a unit dosage comprising about 0.01 µg to about 1,000 µg or more nucleic acid and lipid formulation in a single dose. A vaccine unit dosage can correspond to the unit dosage of nucleic acid molecules, compositions, or pharmaceutical compositions provided herein and that can be administered to a subject. In one aspect, vaccine compositions of the instant disclosure have a unit dosage comprising about 0.01 µg to about 1,000 µg or more nucleic acid and lipid formulation in a single dose. In another aspect, vaccine compositions of the instant disclosure have a unit dosage comprising about 0.01 µg to about 50 µg nucleic acid and lipid formulation in a single dose. In yet another aspect, vaccine compositions of the instant disclosure have a unit dosage comprising about 0.2 µg to about 20 µg nucleic acid and lipid formulation in a single dose.

A dosage form of the composition of this disclosure can be solid, which can be reconstituted in a liquid prior to administration. The solid can be administered as a powder. The solid can be in the form of a capsule, tablet, or gel. In some embodiments, the pharmaceutical composition comprises a nucleic acid lipid formulation that has been lyophilized. In some embodiments, the lyophilized composition may comprise one or more lyoprotectants, such as, including but not necessarily limited to, glucose, trehalose, sucrose, maltose, lactose, mannitol, inositol, hydroxypropyl-β-cyclodextrin, and/or polyethylene glycol. In some embodiments, the lyophilized composition comprises a poloxamer, potassium sorbate, sucrose, or any combination thereof. In specific embodiments, the poloxamer is poloxamer 188. In some embodiments, the lyophilized compositions described herein may comprise about 0.01 to about 1.0% w/w of a poloxamer. In some embodiments, the lyophilized compositions described herein may comprise about 1.0 to about 5.0% w/w of potassium sorbate. The percentages may be any value or subvalue within the recited ranges, including endpoints.

In some embodiments, the lyophilized composition may comprise about 0.01 to about 1.0 % w/w of the nucleic acid molecule. In some embodiments, the composition may comprise about 1.0 to about 5.0 % w/w lipids. In some embodiments, the composition may comprise about 0.5 to about 2.5 % w/w of TRIS buffer. In some embodiments, the composition may comprise about 0.75 to about 2.75 % w/w of NaCl. In some embodiments, the composition may comprise about 85 to about 95 % w/w of a sugar. The percentages may be any value or subvalue within the recited ranges, including endpoints.

In a preferred embodiment, the dosage form of the pharmaceutical compositions described herein can be a liquid suspension of RNA lipid nanoparticles described herein. In some embodiments, the RNA of RNA lipid nanoparticles is a self-replicating RNA. In some embodiments, the RNA of RNA lipid nanoparticles is an mRNA. In some embodiments, the liquid suspension is in a buffered solution. In some embodiments, the buffered solution comprises a buffer selected from the group consisting of HEPES, MOPS, TES, and TRIS. In some embodiments, the buffer has a pH of about 7.4. In some preferred embodiments, the buffer is HEPES. In some further embodiments, the buffered solution further comprises a cryoprotectant. In some embodiments, the cryoprotectant is selected from a sugar and glycerol or a combination of a sugar and glycerol. In some embodiments, the sugar is a dimeric sugar. In some embodiments, the sugar is sucrose. In some preferred embodiments, the buffer comprises HEPES, sucrose, and glycerol at a pH of 7.4. In certain embodiments, the composition comprises a HEPES, MOPS, TES, or TRIS buffer at a pH of about 7.0 to about 8.5. In some embodiments, the HEPES, MOPS, TES, or TRIS buffer may at a concentration ranging from 7 mg/ml to about 15 mg/ml. The pH or concentration may be any value or subvalue within the recited ranges, including endpoints.

In some embodiments, the suspension is frozen during storage and thawed prior to administration. In some embodiments, the suspension is frozen at a temperature below about 70° C. In some embodiments, the suspension is diluted with sterile water during intravenous administration. In some embodiments, intravenous administration comprises diluting the suspension with about 2 volumes to about 6 volumes of sterile water. In some embodiments, the suspension comprises about 0.1 mg to about 3.0 mg RNA/mL, about 15 mg/mL to about 25 mg/mL of an ionizable cationic lipid, about 0.5 mg/mL to about 2.5 mg/mL of a PEG-lipid, about 1.8 mg/mL to about 3.5 mg/mL of a helper lipid, about 4.5 mg/mL to about 7.5 mg/mL of a cholesterol, about 7 mg/mL to about 15 mg/mL of a buffer, about 2.0 mg/mL to about 4.0 mg/mL of NaCl, about 70 mg/mL to about 110 mg/mL of sucrose, and about 50 mg/mL to about 70 mg/mL of glycerol. In some embodiments, a lyophilized RNA-lipid nanoparticle formulation can be resuspended in a buffer as described herein.

In some embodiments, the compositions of the disclosure are administered to a subject such that a RNA concentration of at least about 0.05 mg/kg, at least about 0.1 mg/kg, at least about 0.5 mg/kg, at least about 1.0 mg/kg, at least about 2.0 mg/kg, at least about 3.0 mg/kg, at least about 4.0 mg/kg, at least about 5.0 mg/kg of body weight is administered in a single dose or as part of single treatment cycle. In some embodiments, the compositions of the disclosure are administered to a subject such that a total amount of at least about 0.1 mg, at least about 0.5 mg, at least about 1.0 mg, at least about 2.0 mg, at least about 3.0 mg, at least about 4.0 mg, at least about 5.0 mg, at least about 6.0 mg, at least about 7.0 mg, at least about 8.0 mg, at least about 9.0 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, or at least about 125 mg RNA is administered in one or more doses up to a maximum dose of about 300 mg, about 350 mg, about 400 mg, about 450 mg, or about 500 mg RNA.

Any route of administration can be included in methods provided herein. In some aspects, nucleic acid molecules, i.e., RNA or DNA molecules, compositions, and pharmaceutical compositions provided herein are administered intramuscularly, subcutaneously, intradermally, transdermally, intranasally, orally, sublingually, intravenously, intraperitoneally, topically, by aerosol, or by a pulmonary route, such as by inhalation or by nebulization, for example. In some embodiments, the pharmaceutical compositions described are administered systemically. Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal. In particular embodiments, the intramuscular administration is to a muscle selected from the group consisting of skeletal muscle, smooth muscle and cardiac muscle. In some embodiments, the pharmaceutical composition is administered intravenously.

Pharmaceutical compositions may be administered to any desired tissue. In some embodiments, the RNA delivered is expressed in a tissue different from the tissue in which the lipid formulation or pharmaceutical composition was administered. In preferred embodiments, RNA is delivered and expressed in the liver.

In other aspects, nucleic acid molecules, i.e., RNA or DNA molecules, compositions, and pharmaceutical compositions provided herein are administered intramuscularly.

In some aspects, the subject in which an immune response is induced is a healthy subject. As used herein, the term “healthy subject” refers to a subject not having a condition or disease, including an infectious disease or cancer, for example, or not having a condition or disease against which an immune response is induced. Accordingly, in some aspects, a nucleic acid molecule, composition, or pharmaceutical composition provided herein is administered prophylactically to prevent an infectious disease, for example. A nucleic acid molecule, composition, or pharmaceutical composition provided herein can also be administered therapeutically, i.e., to treat a condition or disease, such as an infection, after the onset of the condition or disease.

As used herein, the terms “treat,” “treatment,” “therapy,” “therapeutic,” and the like refer to obtaining a desired pharmacologic and/or physiologic effect, including, but not limited to, alleviating, delaying or slowing the progression, reducing the effects or symptoms, preventing onset, inhibiting, ameliorating the onset of a diseases or disorder, obtaining a beneficial or desired result with respect to a disease, disorder, or medical condition, such as a therapeutic benefit and/or a prophylactic benefit. “Treatment,” as used herein, includes any treatment of a disease in a mammal, particularly in a human, and includes: (a) preventing the disease from occurring in a subject, including a subject which is predisposed to the disease or at risk of acquiring the disease but has not yet been diagnosed as having it; (b) inhibiting the disease, i.e., arresting its development; and (c) relieving the disease, i.e., causing regression of the disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some aspects, for prophylactic benefit, treatment or compositions for treatment, including pharmaceutical compositions, are administered to a subject at risk of developing a particular disease, or to a subject reporting one or more of the physiological symptoms of a disease, even though a diagnosis of this disease may not have been made. The methods of the present disclosure may be used with any mammal or other animal. In some aspects, treatment results in a decrease or cessation of symptoms. A prophylactic effect includes delaying or eliminating the appearance of a disease or condition, delaying or eliminating the onset of symptoms of a disease or condition, slowing, halting, or reversing the progression of a disease or condition, or any combination thereof.

Nucleic acid molecules, i.e., RNA or DNA molecules, compositions, and pharmaceutical compositions provided herein can be administered once or multiple times. Accordingly, nucleic acid molecules, compositions, and pharmaceutical compositions provided herein can be administered one, two, three, four, five, six, seven, eight, nine, ten, or more times. Timing between two or more administrations can be one week, two weeks, three weeks, four weeks, five weeks, six weeks, seven weeks, eight weeks, nine weeks, weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or more weeks, and any number or range in between. In some aspects, timing between two or more administrations is one month, two months, three months, four months, five months, six months, seven months, eight months, nine months, ten months, 11 months, 12 months, 13 months, 14 months, 15 months, 16 months, 17 months, 18 months, 19 months, 20 months, 21 months, 22 months, 23 months, 24 months, or more months, and any number or range in between. In other aspects, timing between two or more administrations can be one year, two years, three years, four years, five years, six years, seven years, eight years, nine years, ten years, or more years, and any number or range in between, Timing between the first and any subsequent administration can be the same or different. In one aspect, nucleic acid molecules, compositions, or pharmaceutical compositions provided herein are administered once.

More than one nucleic acid molecule, composition, or pharmaceutical composition can be administered in the methods provided herein. In one aspect, two or more nucleic acid molecules, compositions, or pharmaceutical compositions provided herein are administered simultaneously. In another aspect, two or more nucleic acid molecules, compositions, or pharmaceutical compositions provided herein are administered sequentially. Simultaneous and sequential administrations can include any number and any combination of nucleic acid molecules, compositions, or pharmaceutical compositions provided herein. Multiple nucleic acid molecules, compositions, or pharmaceutical compositions that are administered together or sequentially can include transgenes encoding different antigenic proteins or fragments thereof. In this manner, immune responses against different antigenic targets can be induced. Two, three, four, five, six, seven, eight, nine, ten, or more nucleic acid molecules, compositions, or pharmaceutical compositions including transgenes encoding different antigenic proteins or fragments thereof can be administered simultaneously or sequentially. Any combination of nucleic acid molecules, compositions, and pharmaceutical compositions including any combination of transgenes can be administered simultaneously or sequentially. In some aspects, administration is simultaneous. In other aspects, administration is sequential. Timing between two or more administrations can be one week, two weeks, three weeks, four weeks, five weeks, six weeks, seven weeks, eight weeks, nine weeks, weeks, ten weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or more weeks, and any number or range in between. In some aspects, timing between two or more administrations is one month, two months, three months, four months, five months, six months, seven months, eight months, nine months, ten months, 11 months, 12 months, 13 months, 14 months, 15 months, months, 16 months, 17 months, 18 months, 19 months, 20 months, 21 months, 22 months, 23 months, 24 months, or more months, and any number or range in between. In other aspects, timing between two or more administrations can be one year, two years, three years, four years, five years, six years, seven years, eight years, nine years, ten years, or more years, and any number or range in between, Timing between the first and any subsequent administration can be the same or different. Nucleic acid molecules, compositions, and pharmaceutical compositions provided herein can be administered with any other vaccine or treatment.

Following administration of the composition to the subject, the protein product encoded by the RNA of the disclosure (e.g., an antigen) is detectable in the target tissues for at least about one to seven days or longer. The amount of protein product necessary to achieve a therapeutic effect will vary depending on antibody titer necessary to generate an immunity to pathogen or disease such as COVID-19 in the patient. For example, the protein product may be detectable in the target tissues at a concentration (e.g., a therapeutic concentration) of at least about 0.025-1.5 µg/ml (e.g., at least about 0.050 µg/ml, at least about 0.075 µg/ml, at least about 0.1 µg/ml, at least about 0.2 µg/ml, at least about 0.3 µg/ml, at least about 0.4 µg/ml, at least about 0.5 µg/ml, at least about 0.6 µg/ml, at least about 0.7 µg/ml, at least about 0.8 µg/ml, at least about 0.9 µg/ml, at least about 1.0 µg/ml, at least about 1.1 µg/ml, at least about 1.2 µg/ml, at least about 1.3 µg/ml, at least about 1.4 µg/ml, or at least about 1.5 µg/ml), for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 days or longer following administration of the composition to the subj ect.

In some embodiments, the composition described herein may be administered one time. In some embodiments, the composition described herein may be administered two times.

In some embodiments, the composition may be administered in the form of a booster dose, to a subject who was previously vaccinated against coronavirus.

In some embodiments, a pharmaceutical composition of the present disclosure is administered to a subject once per month. In some embodiments, a pharmaceutical composition of the present disclosure is administered to a subject twice per month. In some embodiments, a pharmaceutical composition of the present disclosure is administered to a subject three times per month. In some embodiments, a pharmaceutical composition of the present disclosure is administered to a subject four times per month.

Alternatively, the compositions of the present disclosure may be administered in a local rather than systemic manner, for example, via injection of the pharmaceutical composition directly into a targeted tissue, preferably in a depot or sustained release formulation. Local delivery can be affected in various ways, depending on the tissue to be targeted. For example, aerosols containing compositions of the present disclosure can be inhaled (for nasal, tracheal, or bronchial delivery); compositions of the present disclosure can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing compositions of the present disclosure complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

### Combinations

The RNA, such as a self-replicating RNA or mRNA provided herein, formulations thereof, or encoded proteins described herein may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. Preferably, the methods of treatment of the present disclosure encompass the delivery of pharmaceutical, prophylactic, diagnostic, or imaging compositions in combination with agents that may improve their bioavailability, reduce and/or modify their metabolism, inhibit their excretion, and/or modify their distribution within the body. As a non-limiting example, an RNA molecule of the disclosure may be used in combination with a pharmaceutical agent for immunizing or vaccinating a subject. In general, it is expected that agents utilized in combination with the presently disclosed RNA molecules and formulations thereof be utilized at levels that do not exceed the levels at which they are utilized individually. In some embodiments, the levels utilized in combination will be lower than those utilized individually. In one embodiment, the combinations, each or together may be administered according to the split dosing regimens as are known in the art.

### Ranges

Throughout this disclosure, various aspects can be presented in range format. It should be understood that any description in range format is merely for convenience and brevity and not meant to be limiting. Accordingly, the description of a range should be considered to have specifically disclosed all possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example 1, 2, 2.1, 2.2, 2.5, 3, 4, 4.75, 4.8, 4.85, 4.95, 5, 5.5, 5.75, 5.9, 5.00, and 6. This applies to a range of any breadth.

### EXAMPLE 1

This example describes SARS-CoV-2 RNA vaccine design and construction.

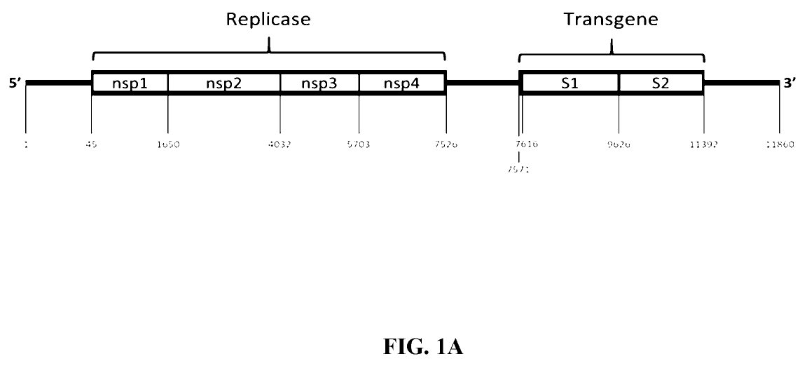

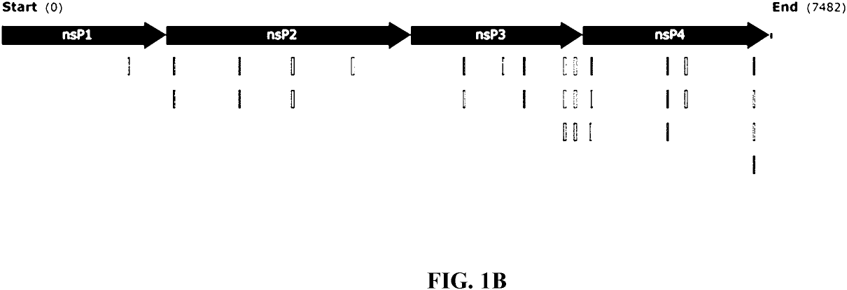

Self-replicating RNA vaccines that encode SARS-CoV-2 spike glycoprotein variants were designed and constructed. FIG. 1 shows a schematic of an exemplary self-replicating RNA (not to scale) of approximately 11,860 kb. Self-replicating RNA vaccines designed for the studies described herein are typically single-stranded molecules that include a 5′ cap, a 5′ untranslated region (UTR), an open reading frame encoding the replicase polyprotein derived from Venezuela Equine Encephalitis Virus (VEEV) that includes the nsP1, nsP2, nsP3, and nsP4 proteins, a transgene 5′ UTR located in the intergenic region that also includes a part of the subgenomic promoter sequence in the negative orientation, an open reading frame of a transgene encoding the primary structure of an antigenic protein, a 3′ UTR, and a poly A tail. The relative location of the open reading frames encoding the replicase polyprotein and a transgene, such as a SARS-CoV-2 spike glycoprotein, are shown (FIG. 1A). The SARS-CoV-2 spike glycoprotein is divided into two domains, S1 and S2. The ACE2 receptor binding domain is located within the S1 domain. The S2 domain includes an intracellular fusion domain, a transmembrane domain, and a cytoplasmic domain. Self-replicating RNA vaccines are generally made from naturally occurring unmodified RNA bases: adenine, guanine, cytosine, and uracil. The 5′ cap of self-replicating RNA vaccines designed as described herein typically has a Cap1 structure (CAP1, m7G(5′)pppA(2′-OMe)pU, with U in RNA denoted as T in DNA and vice versa).

To address the ongoing threat posed by emergence of variant stains of SARS-CoV-2, self-replicating RNA vaccines targeting the D614G and the South African (D614G, D80A, D215G, N501Y, K417N, E484K, A701V point mutations) variants and suitable for delivery using lipid nanoparticles (LNPs) were designed. In addition to these point mutations in the spike protein, sequences encoding the SARS-CoV-2 glycoprotein transgene included codon changes that result in prolines at positions 986 and 987 (K986P and V987P mutations), stabilizing the SARS-CoV-2 glycoprotein in a prefusion conformation and increasing immunogenicity of the S1 receptor binding domain (Baden, et al., 2021, N Engl J Med 384:403-416 & Polack, et al., 2020, N Engl J Med 383:2603-2615; Keech, et al., 2020, N Engl J Med, 383:2320-2332). Furin cleavage sites of SARS-CoV-2 glycoproteins were inactivated by including R682G, R683S, and R685S mutations, thereby changing the RRAR motif at the S1/S2 cleavage junction to GSAS (Wrapp, et al. 2020, Science, 367: 1260-1263). The RRAR motif can also be changed to RRAG or GRAR to inactivate furin cleavage. Transgene sequences encoding variant SARS-CoV-2 spike glycoproteins included in self-replicating RNA vaccines were as follows: SEQ ID NO: 10, encoding South Africa variant B.1.351 (Beta); SEQ ID NO: 11, encoding a SARS-CoV-2 spike glycoprotein having a D614G mutation (B. 1); SEQ ID NO: 12, encoding U.K. variant B.1.1.7 (Alpha); SEQ ID NO: 13, encoding Brazil variant P1 (Gamma).

Self-replicating RNA vaccines included codon-optimized nsP1, nsP2, nsP3, and nsP4 (i.e., replicase) and codon-optimized transgene sequences. Codon-optimized replicase and transgene sequences were included in self-replicating RNA vaccines to increase the amount and duration of XSARS-CoV-2 glycoprotein expression by increasing translation without changing the encoded amino acid sequences. For example, a sequence of SEQ ID NO:6 was obtained using an hCAI algorithm with an input sequence of SEQ ID NO:20 (nucleotides 463-7455), resulting in an intermediate sequence of SEQ ID NO: 185. Use of a luciferase open reading frame (ORF) resulted in a self-replicating RNA sequence of SEQ ID NO: 186, followed by deletion of T7 promoter and BspQ1 restriction enzyme site sequences. Table 6 summarizes steps and parameters of codon-optimization.

TABLE 6

Codon-optimization steps and parameters - nsP1-nsP4

| Name | Design _type | Codon table | Codon_changed | CAI | T_content | ORF2_number | ORF2 _mean _length | ORF3 _number | ORF_ mean_length | Restriction site matches |
|---|---|---|---|---|---|---|---|---|---|---|
| VEEV_hCAI_input sequence | single_step | Genome | 0 | 0.975804612249639 | 0.12998712998713 | 2 | 18.5 | 0 | 0 | 15 (BspQI=0\|B spQI=0\|Nco I=0\|XhoI=0\| AflII=0\|Bsa I=0\|BsaI=l 0\|BsrGI=5) |
| Full_CAI | single_step | Genome | 175 | 1 | 0.121693121693122 | 0 | 0 | 0 | 0 | 26 (BspQI=0\|B spQI=5\|Nco I=2\|XhoI=0\| AflII=0\|Bsa I=0\|BsaI=l 4\|BsrGI=5) |
| Full_CAI_step2_polymerase_motifs | single_step | Genome | 175 | 1 | 0.121693121693122 | 0 | 0 | 0 | 0 | 26 (BspQI=0\|B spQI=5\|Nco I=2\|XhoI=0\| AflII=0\|Bsa I=0\|BsaI=l 4\|BsrGI=5) |
| Full_CAI_step3b_tricodon_repeats | single_step | Genome | 183 | 0.998148600044491 | 0.122694122694123 | 0 | 0 | 0 | 0 | 26 (BspQI=0\|B spQI=5\|Nco I=2\|XhoI=0\| AflII=0\|Bsa I=0\|BsaI=l 4\|BsrGI=5) |
| Full_CAI_step4_homopolymers | single_step | Genome | 199 | 0.995397894405789 | 0.128414128414128 | 0 | 0 | 0 | 0 | 26 (BspQI=0\|B spQI=5\|Nco I=2\|XhoI=0\| AflII=0\|Bsa I=0\|BsaI=l 4\|BsrGI=5) |
| Full_CAI_step6_Restriction_sites | single_step | Genome | 212 | 0.99314463133206 | 0.12970112970113 | 0 | 0 | 0 | 0 | 0 |

The miRanda algorithm (Enright, A. J., John, B., Gaul, U. et al. MicroRNA targets in Drosophila. Genome Biol 5, R1 (2003).doi.org/10.1186/gb-2003-5-1-r1) was then used to identify putative microRNA (miRNA) binding sites in the VEEV non-structural protein coding region (FIG. 1B, Table 6). The sequences of skeletal muscle and dendritic cell miRNA binding sites corresponding to SEQ ID NOs.:54-184 were entered into miRanda to identify putative miRNA binding sites in a self-replicating RNA target sequence that included codon-optimized nsP1, nsP2, nsP3, and nsP4 (i.e., replicase) sequences and a luciferase transgene (SEQ ID NO:186). 15 putative miRNA binding sites representing targets for miRNAs in mouse and human dendritic cells and mouse and human skeletal muscle were identified (FIG. 1B, Table 6).

Exemplary miRNA binding sites in the VEEV nsP1, nsP2, nsP3, and nsP4 regions identified using miRanda are shown in Table 7. The relative positions of putative miRNA binding sites are provided, with nucleotide numbering of nsP1, nsP2, nsP3, and nsP4 serving as a reference.

TABLE 7

Putative miRNA binding sites in the VEEV non-structural protein coding region

| Non-structural protein sequence (nsP, bold) or miRNA target sequence | Position (nt)¹ | miRNA SEQ ID NO |
|---|---|---|
| nsP1 | 1..1605 | |
| hsa-miR-22-3p | 1224..1245 | SEQ ID NO: 101 |
| nsP2 | 1606..3987 | |
| mmu-miR-24-3p | 1670..1691* | SEQ ID NO:131 |
| hsa-miR-24-3p | 1670..1691* | SEQ ID NO:171 |
| mmu-miR-16-5p | 2301..2322^{#} | SEQ ID NO:114 |
| hsa-miR-16-5p | 2301..2322^{#} | SEQ ID NO:175 |
| mmu-miR-16-5p | 2823..2844^{^} | SEQ ID NO:114 |
| hsa-miR-16-5p | 2823..2844^{^} | SEQ ID NO:175 |
| hsa-miR-486-3p | 3406..3428 | SEQ ID NO:81 |
| nsP3 | 3988..5658 | |
| mmu-miR-423-3p | 4494..4514* | SEQ ID NO:72 |
| hsa-miR-423-3p | 4494..4514* | SEQ ID NO:83, SEQ ID NO: 142 |
| hsa-miR-486-5p | 4869..4891 | SEQ ID NO:80 |
| mmu-miR-24-3p | 5079..5100^{#} | SEQ ID NO:131 |
| hsa-miR-24-3p | 5079..5100^{#} | SEQ ID NO:171 |
| hsa-miR-10a-5p | 5470..5493^{^} | SEQ ID NO:102 |
| hsa-miR-10b-5p | 5470..5493^{^} | SEQ ID NO:103 |
| mmu-miR-10a-5p | 5470..5493^{^} | SEQ ID NO:58, SEQ ID NO:112 |
| hsa-miR-10a-5p | 5572..5595^{$} | SEQ ID NO:102 |
| hsa-miR-10b-5p | 5572..5595^{$} | SEQ ID NO:103 |
| mmu-miR-10a-5p | 5572..5595^{$} | SEQ ID NO:58, SEQ ID NO:112 |
| nsP4 | 5659..7479 | |
| mmu-miR-29a-3p | 5718..5739 | SEQ ID NO:113 |
| mmu-miR-423-3p | 5728..5753* | SEQ ID NO:72 |
| hsa-miR-423-3p | 5728..5753* | SEQ ID NO:83, SEQ ID NO: 142 |
| mmu-miR-103-3p | 6470..6491^{#} | SEQ ID NO:128 |
| hsa-miR-103a-3p | 6470..6491^{#} | SEQ ID NO:156 |
| hsa-miR-107 | 6470..6491^{#} | SEQ ID NO:157 |
| mmu-miR-16-5p | 6651..6672^{^} | SEQ ID NO:114 |
| hsa-miR-16-5p | 6651..6672^{^} | SEQ ID NO:175 |
| mmu-miR-10b-5p | 7313..7335^{$} | SEQ ID NO:59 |
| hsa-miR-10b-5p | 7313..7335^{$} | SEQ ID NO:103 |
| mmu-miR-10a-5p | 7318..7335^{$} | SEQ ID NO:58, SEQ ID NO:112 |
| hsa-miR-10a-5p | 7318..7335^{$} | SEQ ID NO:102 |

¹Symbols *, #, ^, and $ indicate identical nucleotide positions for miRNAs within each of the non-structural coding sequences encoding nsP1, nsP2, nsP3, and nsP4

Identified seed sequences of putative miRNA target sites were manually mutated in silico to synonymous codons to eliminate or reduce miRNA binding. Elimination of miRNA binding sites was confirmed using miRanda. Without being limited by theory, mutation of miRNA binding sites to eliminate or reduce miRNA binding based on predictions using miRanda should result in increased expression of sequences encoding VEEV non-structural proteins. Codon-optimized sequences encoding SARS-CoV-2 spike glycoprotein and its variants were introduced into self-replicating RNA backbones having codon-optimized nsP1-4 sequences and mutated miRNA binding sites.

Exemplary mutations of putative miRNA binding sites in the nsP1-nsP4 coding region of self-replicating RNAs are summarized in Table 8. Mutations made in 15 putative miRNA binding sites identified in the VEEV nsP1, nsP2, nsP3, and nsP4 regions are shown. The relative positions of putative miRNA binding sites are provided, with nucleotide numbering of nsP1, nsP2, nsP3, and nsP4 serving as a reference and point mutations shown below putative miRNAs and their positions in bold italics.

TABLE 8

Exemplary mutations of putative miRNA binding sites in the VEEV nsP1, nsP2, nsP3, and nsP4 regions

| nsP | miRNA Binding Site (Number) | miRNA | Position (nt)¹ |
|---|---|---|---|
| nsP1 | | | 1..1605 |
| | 1 | hsa-miR-22-3p | 1224..1245 |
| | | hsa-miR-22-3p | 1224..1245 |
| | | A to C | 1237 |
| | | G to A | 1239 |
| nsP2 | | | 1606..3987 |
| | 2 | mmu-miR-24-3p | 1670..1691 |
| | | hsa-miR-24-3p | 1670..1691 |
| | | G to C | 1686 |
| | 3 | mmu-miR-16-5p | 2301..2322 |
| | | hsa-miR-16-5p | 2301..2322 |
| | | G to C | 2319 |
| | 4 | mmu-miR-16-5p | 2823..2844 |
| | | hsa-miR-16-5p | 2823..2844 |
| | | G to C | 2838 |
| | 5 | hsa-miR-486-3p | 3406..3428 |
| | | C to A | 3426 |
| nsP3 | | | 3988..5658 |
| | 6 | mmu-miR-423-3p | 4494..4514 |
| | | hsa-miR-423-3p | 4494..4514 |
| | | C to G | 4509 |
| | 7 | hsa-miR-486-5p | 4869..4891 |
| | | A to C | 4888 |
| | 8 | mmu-miR-24-3p | 5079..5100 |
| | | hsa-miR-24-3p | 5079..5100 |
| | | C to G | 5091 |
| | | T toC | 5094 |
| | 9 | hsa-miR-10a-5p | 5470..5493 |
| | | hsa-miR-10b-5p | 5470..5493 |
| | | mmu-miR-10a-5p | 5470..5493 |
| | | A to C | 5488 |
| | 10 | hsa-miR-10a-5p | 5572..5595 |
| | | mmu-miR-10a-5p | 5572..5595 |
| | | hsa-miR-10b-5P | 5572..5595 |
| | | mmu-miR-10b-5P | 5572..5595 |
| | | A to C | 5590 |
| nsP4 | | | 5659..7479 |
| | 11 | mmu-miR-29a-3p | 5718..5739 |
| | | G to C | 5736 |
| | 12 | mmu-miR-423-3p | 5728..5753 |
| | | hsa-miR-423-3p | 5728..5753 |
| | | C to G | 5748 |
| | 13 | mmu-miR-103-3p | 6470..6491 |
| | | hsa-miR-103a-3p | 6470..6491 |
| | | hsa-miR-107 | 6470..6491 |
| | | G to C | 6489 |
| | 14 | mmu-miR-16-5p | 6651..6672 |
| | | hsa-miR-16-5p | 6651..6672 |
| | | G to C | 6666 |
| | 15 | mmu-miR-10b-5p | 7313..7335 |
| | | hsa-miR-10b-5p | 7313..7335 |
| | | mmu-miR-10a-5p | 7318..7335 |
| | | hsa-miR-10a-5p | 7318..7335 |
| | | A to C | 7330 |

¹Point mutations within miRNA binding sites are shown in bold italics

Exemplary features of self-replicating RNA vaccines are summarized in Table 9.

TABLE 9

Exemplary features of self-replicating RNA vaccines

| | | |
|---|---|---|
| Feature | Sequence and/or Domain | Effect |
| Codon Optimization | Replicon Spike Glycoprotein | Increased amount and duration of spike glycoprotein expression due to increased translation efficiency with no change to amino acid sequence |
| Prefusion Stabilization | Spike Glycoprotein K986P; V987P | Increased immunogenicity of S1 receptor binding domain |
| Inactivation of Furin Cleavage Site | Spike Glycoprotein R682G; R683S, R685S (codons for RRAR changed to GSAS) | Increased immunogenicity of spike glycoprotein |
| G Clade SARS-CoV-2 Variant (B.1) | Spike Glycoprotein D614G | Expands neutralizing antibody titers to G clade spike glycoprotein variant while maintaining immunogenicity to Wuhan spike glycoprotein |
| B.1.351 SARS-CoV-2 Variant | Spike Glycoprotein D614G, D80A, D215G, K417N, A701V, N501Y, E484K | Increased neutralizing antibody titers to B.1.351 South African spike glycoprotein variant |

Table 10 summarizes features of self-replicating RNA constructs encoding SARS-CoV-2 South Africa and D614G spike glycoprotein variants.

TABLE 10

Features of self-replicating RNA constructs encoding SARS-CoV-2 South Africa and D614G spike glycoprotein variants

| | | |
|---|---|---|
| RNA/Feature | Self-Replicating RNA Encoding SARS-CoV-2 D614G spike glycoprotein variant (also designated mRNA-2105 or ARCT-154) | Self-Replicating RNA Encoding SARS-CoV-2 South Africa spike glycoprotein variants (also designated South Africa mRNA-2106 or ARCT-165) |
| RNA Construct Replicon Spike | Codon optimization designed to minimize the number of uridines for both replicon and spike glycoprotein and at the same time select codons most frequently used by human cells* | Codon optimization designed to minimize the number of uridines for both replicon and spike glycoprotein and at the same time select codons most frequently used by human cells* |
| Additional modifications | microRNA target changed to reduce RNAtranscript turnover and to mitigate miRNA-mediated translation repression** | microRNA target changed to reduce RNAtranscript turnover and tomitigate miRNA-mediated translation repression ** |
| Spike glycoprotein conformation | Prefusion stabilized by the following changing codon sequences for amino acids at positions 986 and 987 to encode for prolines*** | Prefusion stabilized by the following changing codon sequences 986 and 987 to encode for prolines*** |
| Furin Cleavage Site | Codons for RRAR changed to GSAS to prevent furin proteolytic cleavage at S1/S2**** | Codons for RRAR changed to GSAS to prevent furin proteolytic cleavageat S1/S2**** |
| Point Mutations | D614G | D614G, D80A, D215G, N501Y, K417N, E484K, A701V |

*The codon optimization method reduces the number of uridines in the RNA transcript. Without being limited by theory, the purpose is to reduce innate immune activation and increase translation efficiency of open reading frames while maintaining a high level of antigen expression. These RNA sequence changes by the optimization method do not alter the amino acid sequence of the replicon or antigen upon translation of the RNA transcript.
**Change in sequence to eliminate a potential micro RNA target sequence (in mouse and human dendritic cells and skeletal muscle cells) may decrease the turnover rate of the transcript and/or reduce miRNA-mediated translation repression, thereby increasing antigen expression.
***The two proline substitutions at codons for amino acids at positions 986 and 987 in the spike glycoprotein result in the ACE2 receptor binding domain of the spike glycoprotein to be inthe “Up” or unburied state vs. “down” or buried state (Corbett et. al 2020 bioRxiv doi: doi.org/10.1101/2020.06.11.145920, Nature. 2020 Oct, 586(7830): 567-571; Sahin et. al. 2020 medRxiv doi: doi.org/10.1101/2020.12.09.20245175, Nature. 2021, 595, 572-577).
****Changing the RRAR sequence at the S1/S2 domain to GSAS prevents furin cleavage. Furin cleavage at the S1 and S2 domains results in only ionic, hydrophobic and Van der Waals radii association of the S1 domain with the S2 domain, i.e., non-covalent interaction. Inactivation of the cleavage site increases antibody neutralization titers (Kalnin, et. al. 2020 bioRxiv doi: doi.org/10.1101/2020.10.14.337535; npj Vaccines 6, 61 (2021)).

In addition to self-replicating RNA vaccines encoding SARS-CoV-2 South Africa and D614G spike glycoprotein variants (e.g., SEQ ID NO:1 and SEQ ID NO:2, respectively, for the full-length self-replicating RNA sequence, with U in RNA shown as T in DNA and vice versa), self-replicating RNA vaccines encoding SARS-CoV-2 UK B.1.1.7 and Brazil P.1 spike glycoprotein variants were designed (SEQ ID NO:3 and SEQ ID NO:4, respectively, for the full-length self-replicating RNA sequence, with U in RNA shown as T in DNA and vice versa). Sequences of construct features, such as 5′ UTR, 3′ UTR, and transgene sequences, are provided below in addition to full-length construct sequences.

Messenger RNA (mRNA) vaccines that encode an antigenic protein such as a SARS-CoV-2 spike glycoprotein or another viral glycoprotein were also designed. mRNA vaccines typically include a 5′ UTR, an open reading frame encoding an antigenic protein, a 3′ UTR, and a poly-A tail. Other sequence elements of mRNA vaccines generally include a Kozak sequence and translational enhancers located in untranslated regions, either the 5′ UTR, the 3′ UTR, or both.

mRNA vaccines encoding SARS-CoV-2 South Africa and D614G spike glycoprotein variants were designed and constructed (SEQ ID NO:29 and SEQ ID NO:32, respectively, for the full-length mRNA sequences, with U in RNA shown as T in DNA and vice versa). mRNA constructs included a 5′ TEV UTR (SEQ ID NO: 35) and a 3′ Xenopus beta-globin (Xbg) UTR (SEQ ID NO:36 with poly-A tail; SEQ ID NO:37 without poly-A tail).

Self-replicating RNA and mRNA vaccines encoding any SARS-CoV-2 spike glycoprotein variant, any SARS-CoV-2 spike glycoprotein having any mutation or any combination of mutations, or any other viral glycoprotein can be designed and constructed similar to the constructs described above. SARS-CoV-2 spike glycoprotein variants, SARS-CoV-2 spike glycoproteins having a mutation or a combination of mutations, or any other viral glycoproteins can be included in self-replicating RNA and mRNA vaccines having a backbone that includes any combination of the features described above. Exemplary SARS-CoV-2 spike glycoprotein variants and SARS-CoV-2 spike glycoprotein mutations that can be encoded are shown in Table 11. Additional SARS-CoV-2 spike glycoprotein variants can be found at, e.g., outbreak.info/situation-reports. Exemplary RNA molecules that encode a hemagglutinin (HA) of influenza virus were designed and prepared, including a self-replicating RNA having a sequence of SEQ ID NO:40 and an mRNA having a sequence of SEQ ID NO:48.

TABLE 11

Exemplary SARS-CoV-2 Spike Glyoproteins^{#}

| SARS-CoV-2 Glycoprotein WHO Label (Variant/First Identified) | Exemplary Mutation(s) |
|---|---|
| Variants of Concern or with Potential to Become Variant of Concern | |
| Alpha (B.1.1.7; UK) | 69del, 70del, 144del, (E484K*), (S494P*), N501Y, A570D, D614G, P681H, T716I, S982A, D1118H (K1191N*) |
| Beta (B.1.351; South Africa) | D80A, D215G, 241del, 242del, 243del, K417N, E484K, N501Y, D614G, A701V |
| Delta (B.1.617.2; India) | T19R, (V70F*), T95I, G142D, E156-, F157-, R158G, (A222V*), (W258L*), (K417N*), L452R, T478K, D614G, P681R, D950N |
| Gamma (P1; Brazil, Japan) | L18F, T20N, P26S, D138Y, R190S, K417T, E484K, N501Y, D614G, H655Y, T1027I |
| Lambda (C.37; Peru) | G75V, T76I, Δ246-252, L452Q, F490S, D614G, T859N |
| Variants of Interest | |
| Epsilon (B.1.427; United States -California) | L452R, D614G |
| Epsilon (B.1.429; United States - California) | S13I, W152C, L452R, D614G |
| Eta (B.1.525; United Kingdom/Nigeria) | A67V, 69del, 70del, 144del, E484K, D614G, Q677H, F888L |
| Iota (B. 1.526; United States - New York) | L5F, (D80G*), T95I, (Y144-*), (F157S*), D253G, (L452R*), (S477N*), E484K, D614G, A701V, (T859N*), (D950H*), (Q957R*) |
| Kappa (B.1.617.1; India) | (T95I), G142D, E154K, L452R, E484Q, D614G, P681R, Q1071H |
| N/A (B.1.617.3; India) | T19R, G142D, L452R, E484Q, D614G, P681R, D950N |
| Zeta (P.2; Brazil) | E484K, (F565L*), D614G, V1176F |
| Theta (P.3; Philippines) | E484K, N501Y, D614G, P681H, E1092K, H1101Y, V1176F |

^{#}cdc.gov/coronavirus/2019-ncov/variants/variant-info.html; Robertson, Sally (27 Jun. 2021). “Lambda lineage of SARS-CoV-2 has potential to become variant of concern.” news-medical.net; outbreak.info/situation-reports.
(*) = not detected in all sequences

### EXAMPLE 2

This example describes expression and potency of SARS-CoV-2 RNA vaccine constructs.

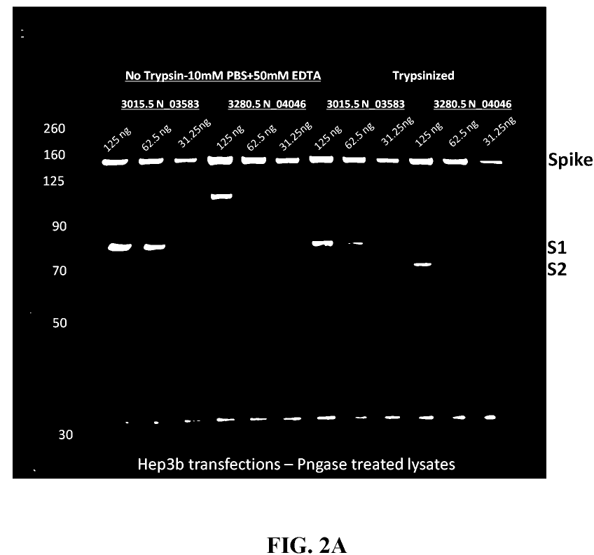

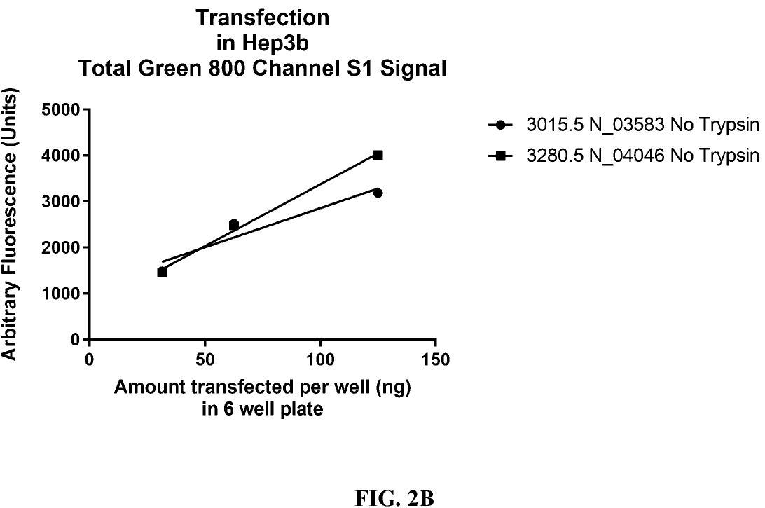

Initial experiments were performed to establish assay conditions to determine protein expression from SARS-CoV-2 RNA vaccine constructs. Hep3b cells were transfected with 125 ng, 62.5 ng, or 31.25 ng of self-replicating RNA encoding a SARS-CoV-2 Wuhan spike glycoprotein (mARM3015; SEQ ID NO:18) or a self-replicating RNA encoding a SARS-CoV-2 D614G spike glycoprotein variant (mARM3280; SEQ ID NO:2) (FIG. 2A). Throughout this disclosure, RNAs designated with a suffix of “.1” were synthesized in the presence of N¹-methylpseudouridine (N1MPU), resulting in 100% of the uridines being N1MPU, while RNAs designated with a suffix of “.5” did not include modified nucleotides, unless otherwise indicated. Cells were harvested by scraping into buffer that included 10 mM PBS and 50 mM EDTA or by trypsinization. Total protein was isolated and protein concentration determined by BCA assay performed in duplicate, with duplicates yielding comparable results. Proteins were separated by polyacrylamide gel electrophoresis and transferred to membranes at 45 V for 1.5 hours for Western blotting using an antibody detecting the SARS-CoV-2 spike glycoprotein.

Total protein was comparable for cells transfected with SARS-CoV-2 vaccine constructs encoding either a SARS-CoV-2 Wuhan spike glycoprotein or a SARS-CoV-2 D614G spike glycoprotein variant. Similar banding patterns were observed for the self-replicating RNA vaccine construct expressing the SARS-CoV-2 Wuhan spike glycoprotein for cells harvested with or without trypsinization, with bands corresponding to full-length spike and S1 and S2 domains (FIG. 2A, arrows). By contrast, bands corresponding to S1 and S2 were observed for protein extracts prepared from cells harvested by trypsinization that were not observed for protein extracts prepared from cells without trypsinization (FIG. 2A) for the SARS-CoV-2 D614G spike glycoprotein variant. Without being limited by theory, these results indicate that harvesting of cells by trypsinization may alter the banding pattern seen for the SARS-CoV-2 D614G spike glycoprotein variant, while trypsinization has no detectable effect on the SARS-CoV-2 Wuhan spike glycoprotein. Unlike the SARS-CoV-2 D614G spike glycoprotein variant expressed from the self-replicating RNA construct of SEQ ID NO:2, the SARS-CoV-2 Wuhan spike glycoprotein expressed from the self-replicating RNA construct of SEQ ID NO: 18 did not include the two proline modifications that stabilize the spike glycoprotein in a prefusion conformation and the inactivated furin cleavage site (described in Example 1, above). Without being limited by theory, these differences may contribute to trypsin sensitivity in addition to variant-specific point mutations.

FIG. 2B shows quantitation of SARS-CoV-2 spike protein expressed from the indicated construct based on S1 signal using protein extracts prepared from cells that were transfected as described above and harvested without trypsinization. Comparable levels of SARS-CoV-2 spike protein was seen for the constructs expressing the SARS-CoV-2 Wuhan glycoprotein or D614G spike glycoprotein variant.

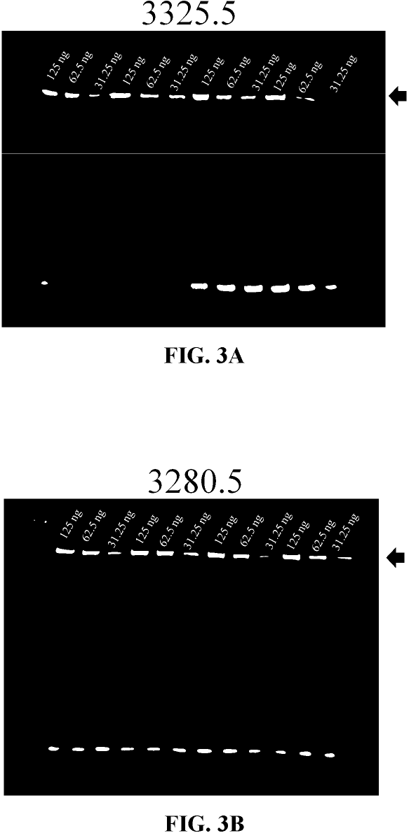

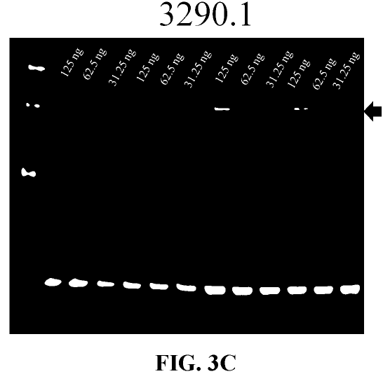

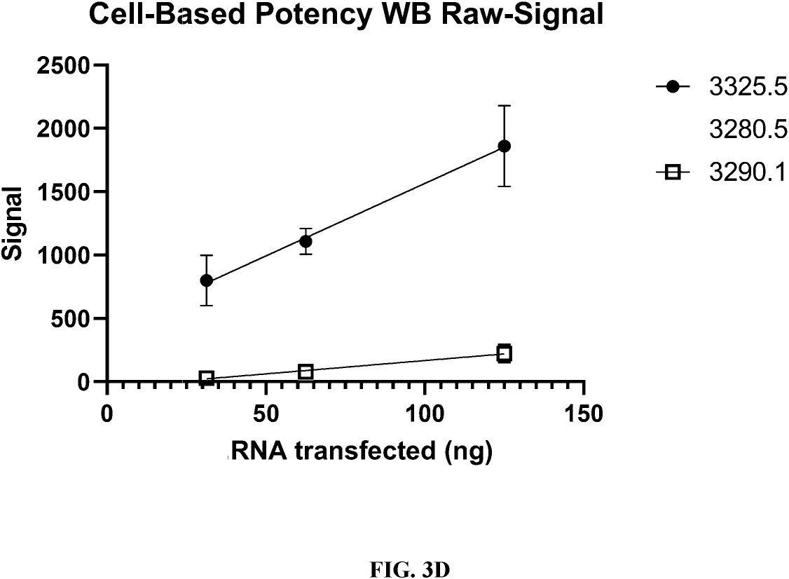

Potency of self-replicating RNA vaccine constructs encoding SARS-CoV-2 D614 (mARM3280; SEQ ID NO:2) or South Africa (mARM3326; SEQ ID NO:1) variant spike glycoproteins was studied next. An mRNA construct encoding the SARS-CoV-2 D614 variant spike glycoprotein (mARM3290; SEQ ID NO:32) was also included in these studies (FIGS. 3A-C). 700,000 Hep3B cells were plated in 6-well plates the day before transfection, followed by transfection with 31.3 ng, 62.5 ng, or 125 ng of self-replicating RNA or mRNA in quadruplicate. The day after transfection, cells were treated with EDTA and scraped, followed by sonication to lyse cells in the absence of trypsin. Lysates were treated with PNGase and S1 and S2 protein levels were determined by Western blot using an anti-S1 rabbit polyclonal antibody (Sino Biological, 40150-T62-COV2). Western blot results for the indicated constructs are shown in FIGS. 3A-C, with full-length spike glycoprotein indicated by the arrow. FIG. 3D shows quantitation of SARS-CoV-2 spike glycoprotein expression detected (y-axis) from the indicated construct as a function of amount of RNA transfected (x-axis). Data analysis of cell-based potency for four replicates is shown in Table 12, expressed as relative potency as compared to references representing previously characterized constructs.

TABLE 12

Analysis of Cell Based Potency

| Construct | Slope + SE | Goodness of Fit (R-Square) | Relative Potency of Samples vs References |
|---|---|---|---|
| 3325.5 | 11.44 ± 0.52 | 0.998 | 148% |
| 3280.5 | 10.89 ± 3.34 | 0.914 | 149% |
| 3290.1 | 2.1 ± 0.17 | 0.993 | 163% |

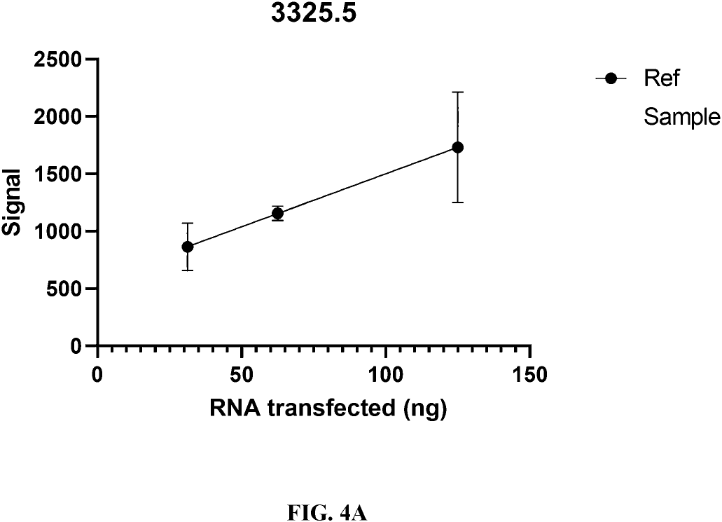

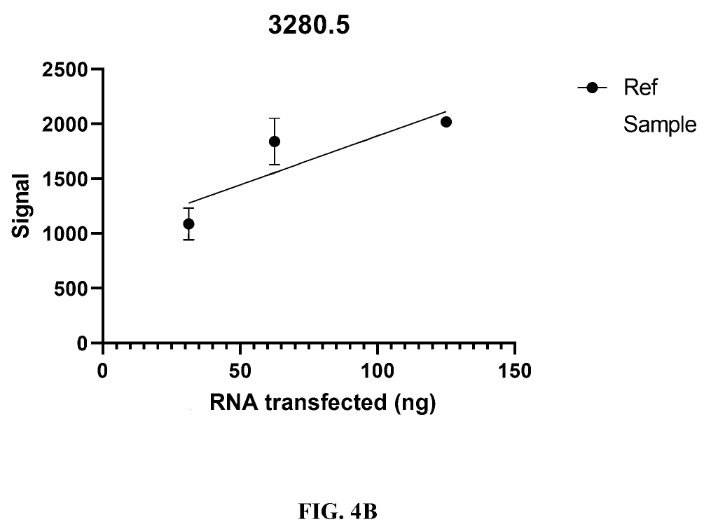

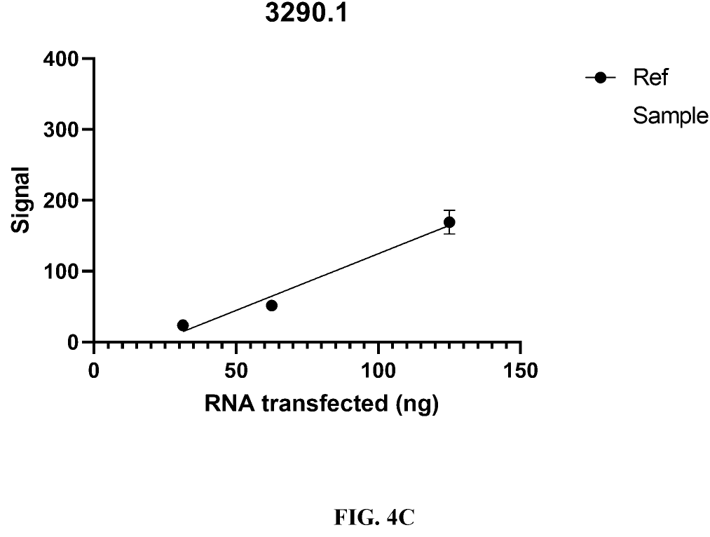

Results of analysis comparing signals obtained for references, i.e., internally characterized constructs, and samples (y-axis) as a function of RNA amount transfected (x-axis) for the indicated construct are shown in FIGS. 4A-C, with data shown in Tables 13-15.

TABLE 13

Comparison of Reference to Sample 3325.5

| Construct/Sample | Slope + SE (Mean) | Goodness of Fit (R-Square) | Relative Potency of Samples vs References (two each) |
|---|---|---|---|
| 3325.5 | 13.65 ± 1.09 | 0.993 | 148% |
| Reference | 9.23 ± 0.03 | 1 | N/A |

TABLE 14

Comparison of Reference to Sample 3380.5

| Construct/Sample | Slope + SE (Mean) | Goodness of Fit (R-Square) | Relative Potency of Samples vs References (two each) |
|---|---|---|---|
| 3380.5 | 13.03 ± 2.00 | 0.977 | 149% |
| Reference | 8.75 ± 4.67 | 0.778 | N/A |

TABLE 15

Comparison of Reference to Sample 3290.1

| Construct/Sample | Slope + SE (Mean) | Goodness of Fit (R-Square) | Relative Potency of Samples vs References (two each) |
|---|---|---|---|
| 3290.1 | 2.60 ± 0.09 | 0.999 | 163% |
| Reference | 1.59 ± 0.24 | 0.977 | N/A |

These results show efficient expression and potency for self-replicating RNA and mRNA constructs encoding SARS-CoV-2 spike glycoprotein variants.

### EXAMPLE 3

This example describes immunogenicity of RNA vaccines encoding SARS-CoV-2 spike glycoprotein variants in mice.

To determine immunogenicity of RNA constructs encoding SARS-CoV-2 spike glycoprotein variants, Balb/C female mice were administered the indicated RNA as shown in Table 16.

TABLE 16

Administration of RNA Vaccines to Mice

| RNA / SARS-CoV-2 glycoprotein | Description | Dose (µg) | Administration Route | Number of Mice | Boost |
|---|---|---|---|---|---|
| PBS | N/A | N/A | i.m.; rectus femoris (bilateral administration) | 5 | N/A |
| Self-replicating RNA / D614G | SEQ ID NO:2 (ARCT-154 / mARM3280.5); pre-fusion stabilized, furin cleavage site inactivated | 2 | i.m.; rectus femoris (bilateral administration) | 5 | No |
| Self-replicating RNA / Wuhan (wild-type) | SEQ ID NO: 18 (ARCT-021 / mARM3015.5) | 2 | i.m.; rectus femoris (bilateral administration) | 5 | No |
| Self-replicating RNA / South Africa variant (B.1.351; Beta) | SEQ ID NO:1 (ARCT-165 / mARM3325.5); pre-fusion stabilized, furin cleavage site inactivated | 2 | i.m.; rectus femoris (bilateral administration) | 5 | No |
| mRNA/D614G | SEQ ID NO:32 (ARCT-143 / mARM3290.1); pre-fusion stabilized, furin cleavage site inactivated | 2 | i.m.; rectus femoris (bilateral administration) | 5 | Day 28 |
| mRNA/D614G | SEQ ID NO:32 (ARCT-143 / mARM3290.1); pre-fusion stabilized, furin cleavage site inactivated | 15 | i.m.; rectus femoris (bilateral administration) | 5 | Day 28 |

Serum was obtained at day 0 (pre-bleed) and at days 14, 28, 42 and 56 after the first immunization. Serum was probed simultaneously for responses to four SARS-CoV-2 spike glycoprotein variants: SARS-CoV-2 spike (Wuhan, wild-type), SARS-CoV-2 spike (P.1, Brazil, Gamma), SARS-CoV-2 spike (B. 1.351, South Africa, Beta) and SARS-CoV-2 spike (B.1.1.7, UK, Alpha). V-PLEX SARS-CoV-2 Panel 5 IgG and ACE2 Kits from MSD (Cat# K15429U and K15432U) were used for measuring serum IgG antibody levels. For total IgG binding, serum was diluted 1:10,000 in kit Dilution 100 buffer (MSD, Cat# R50AA). A goat anti-mouse IgG antibody (MSD, Cat# R32AC) was used for signal detection. Results were reported as AU/ml using a human serum-based reference standard. For surrogate virus neutralization test (sVNT) assays, serum was diluted 1:200 in the kit Dilution 100 buffer (MSD, Cat# R50AA), and results were reported as ACE2-binding percent inhibition using the following formula: 1- (average sample signal/average Diluent 100 only signal) ×100. ACE2 Calibration Reagent (included in the MSD kit) was used a positive control, showing 100% inhibition.

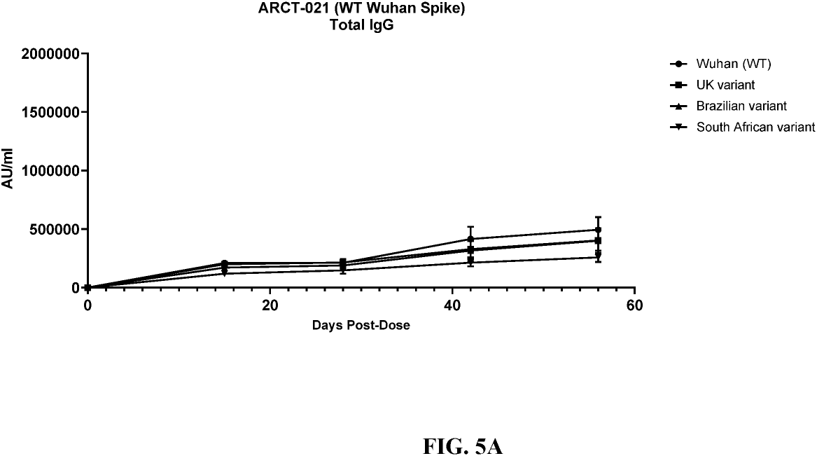

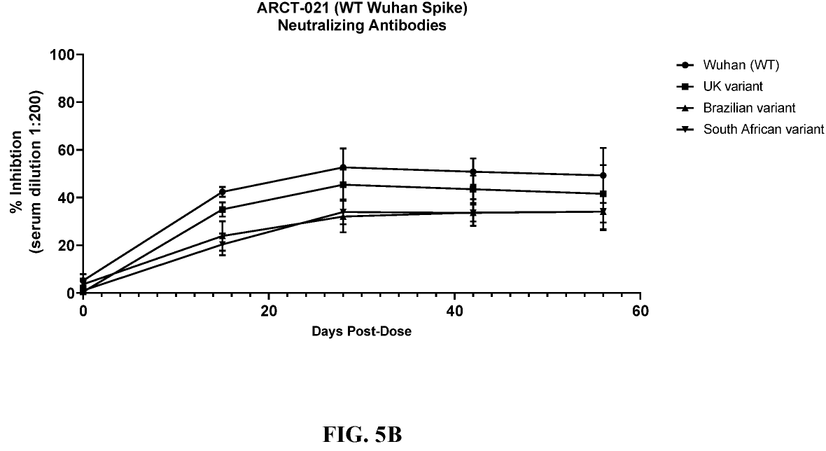

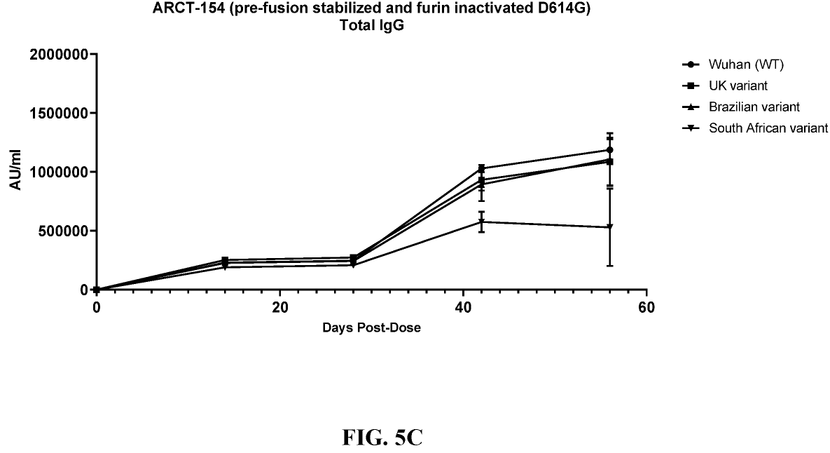

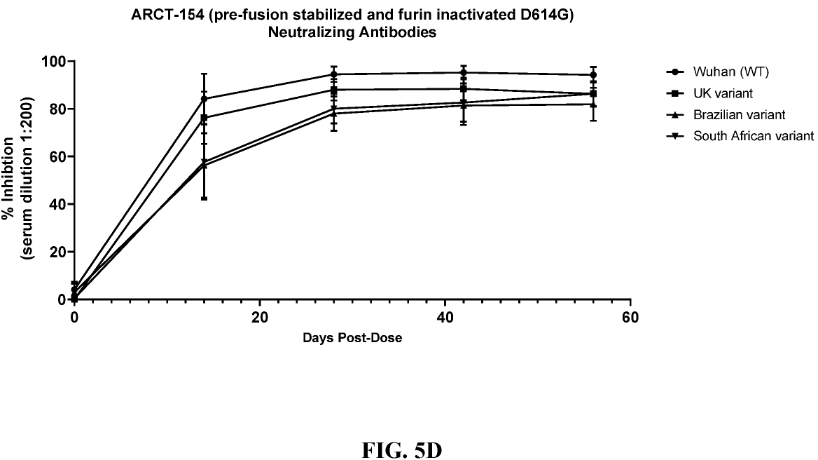

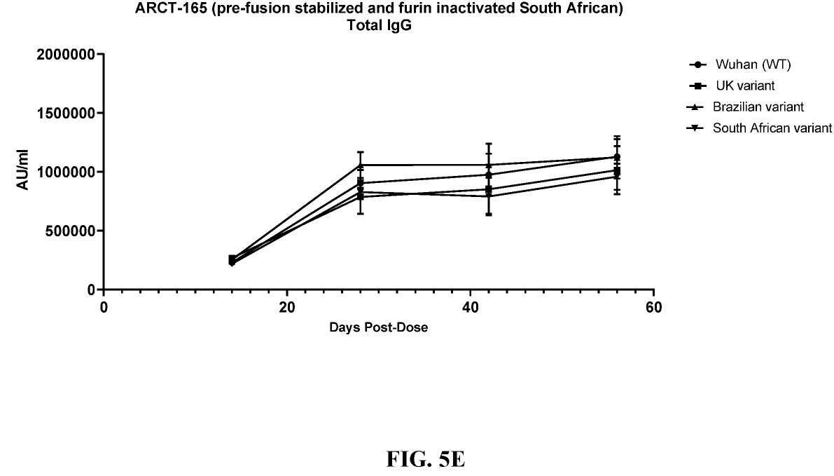

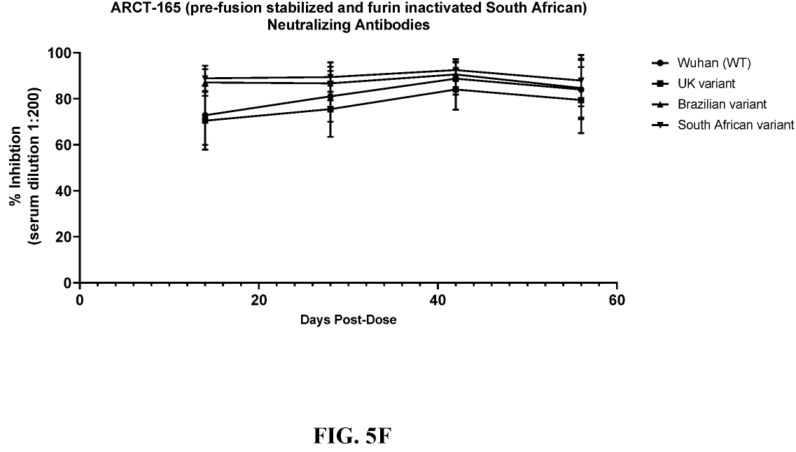

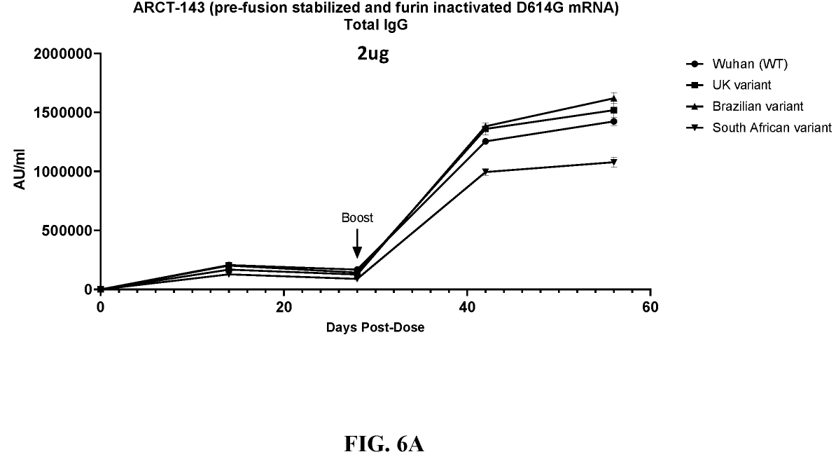

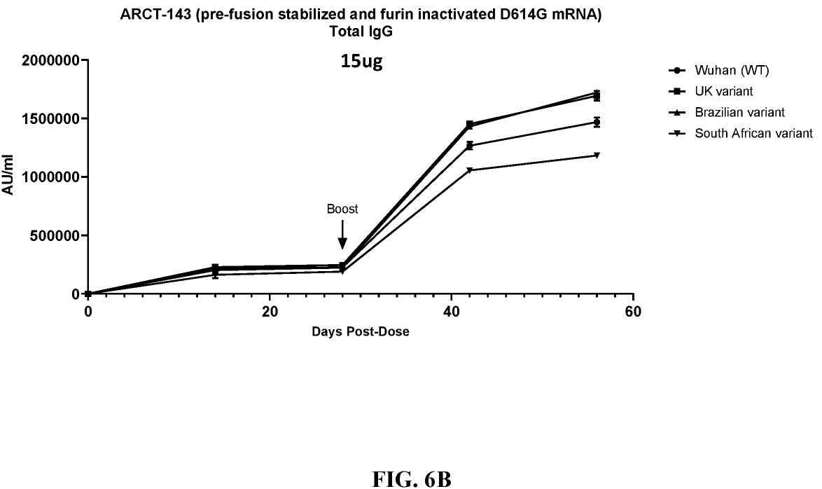

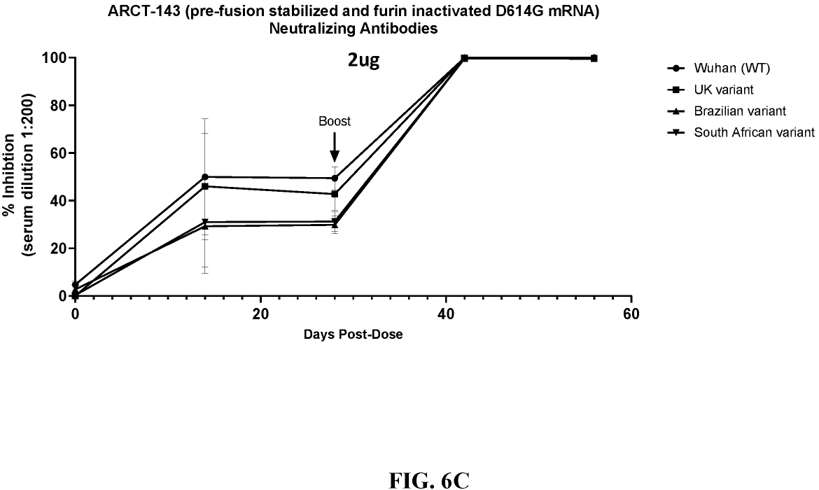

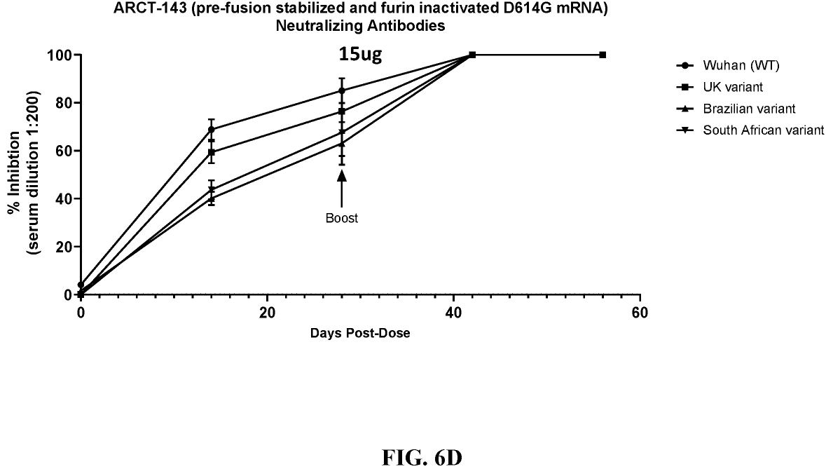

Results for total IgG and neutralizing antibodies upon immunization with lipid-formulated self-replicating RNA encoding a wild-type (Wuhan) SARS-CoV-2 spike glycoprotein (SEQ ID NO:18; ARCT-021 / mARM3015.5), a SARS-CoV-2 D614 variant spike glycoprotein (SEQ ID NO:2; ARCT-154 / mARM3280), or a SARS-CoV-2 South Africa variant glycoprotein (SEQ ID NO:1; ARCT-165 / mARM3325) are shown in FIGS. 5A-F. Results for total IgG and neutralizing antibodies upon immunization with 2 µg or 15 µg of lipid-formulated mRNA encoding a SARS-CoV-2 D614 variant spike glycoprotein (SEQ ID NO:32; ARCT-143 / mARM3290) are shown in FIGS. 6A-D.

Immunization of mice with lipid-formulated self-replicating RNA encoding a wild-type (Wuhan) SARS-CoV-2 spike glycoprotein (SEQ ID NO: 18; ARCT-021 / mARM3015.5; FIGS. 5A-B), a SARS-CoV-2 D614 variant spike glycoprotein (SEQ ID NO:2; ARCT-154 / mARM3280; FIGS. 5C-D), or a SARS-CoV-2 South Africa variant spike glycoprotein (SEQ ID NO: 1; ARCT-165 / mARM3325; FIGS. 5E-F) elicited a SARS-CoV-2 specific IgG and neutralizing antibody responses against both wild-type and variant SARS-CoV-2 spike glycoproteins, including Wuhan (wild-type), UK (B.1.1.7; Alpha), Brazil (P1; Gamma), and South Africa (B. 1.351; Beta) variants. Greater IgG and neutralizing antibody responses to both wild-type and variant SARS-CoV-2 spike glycoproteins were seen upon immunization with lipid-formulated self-replicating RNA encoding a SARS-CoV-2 D614 variant spike glycoprotein (SEQ ID NO:2; ARCT-154 / mARM3280; FIGS. 5C-D) or a SARS-CoV-2 South Africa variant spike glycoprotein (SEQ ID NO: 1; ARCT-165 / mARM3325; FIGS. 5E-F) as compared to immunization with lipid-formulated self-replicating RNA encoding a wild-type (Wuhan) SARS-CoV-2 spike glycoprotein (SEQ ID NO: 18; ARCT-021 / mARM3015.5; FIGS. 5A-B).

Immunization of mice with 2 µg or 15 µg of lipid-formulated mRNA encoding a SARS-CoV-2 D614 variant spike glycoprotein (SEQ ID NO: 32; ARCT-143 / mARM3290) that included a boost at day 28 also resulted in higher specific IgG levels against both wild-type and different variant SARS-CoV-2 spike glycoproteins as compared to immunization with self-replicating RNA encoding a wild-type (Wuhan) SARS-CoV-2 spike glycoprotein (SEQ ID NO:18; ARCT-021 / mARM3015.5; FIG. 5A; FIGS. 6A-B). Neutralizing antibody levels upon immunization with mRNA encoding SARS-CoV-2 D614G spike glycoprotein were likewise greater as compared to neutralizing antibody levels seen upon immunization with self-replicating RNA encoding wild-type (Wuhan) SARS-CoV-2 spike glycoprotein (FIG. 5B; FIGS. 6C-D).

These results show that immunization of mice with self-replicating RNA or mRNA encoding SARS-CoV-2 variant D614G or variant South Africa spike glycoproteins elicits effective humoral immune responses, including neutralizing antibodies that were effective against wild-type and numerous SARS-CoV-2 variant glycoproteins.

### EXAMPLE 4

This example describes immunogenicity of RNA vaccines encoding SARS-CoV-2 spike glycoprotein variants in non-human primates (NHPs).

To determine immunogenicity of RNA constructs encoding SARS-CoV-2 spike glycoprotein variants in NHPs, the indicated RNA constructs were administered as shown in Table 17.

TABLE 17

Administration of RNA Vaccines to NHPs

| RNA / SARS-CoV-2 glycoprotein | Description | Dose Level (µg) | Dose Volume (ml) | Dose Concentration (µg/ml) | Administration Days | Number of NHPs (Males) |
|---|---|---|---|---|---|---|
| Control (PBS) | N/A | N/A | 0.5 | N/A | 1 and 29 | 2 |
| Self-replicating RNA / Wuhan | SEQ ID NO: 18 (ARCT-021 / mARM3015.5) | 7.5 | 0.5 | 15 | 1 and 29 | 4 |
| Self-replicating RNA / D614G | SEQ ID NO:2 (ARCT-154 / mARM3280.5); pre-fusion stabilized, furin cleavage site inactivated | 7.5 | 0.5 | 15 | 1 and 29 | 4 |
| Self-replicating RNA / South Africa variant | SEQ ID NO:1 (ARCT-165 / mARM3325.5); pre-fusion stabilized, furin cleavage site inactivated | 7.5 | 0.5 | 15 | 1 and 29 | 4 |
| mRNA/D614G | SEQ ID NO:32 (ARCT-143 / mARM3290.1); pre-fusion stabilized, furin cleavage site inactivated | 30 | 0.5 | 60 | 1 and 29 | 4 |

Serum was obtained at day 0 (pre-bleed) and at days 15, 29, and 43 after the first immunization. Serum was probed simultaneously for responses to four SARS-CoV-2 spike glycoprotein variants: SARS-CoV-2 spike (Wuhan, wild-type), SARS-CoV-2 spike (P.1, Brazil, Gamma), SARS-CoV-2 spike (B.1.351, South Africa, Beta) and SARS-CoV-2 spike (B.1.1.7, UK, Alpha). V-PLEX SARS-CoV-2 Panel 5 IgG and ACE2 Kits from MSD (Cat# K15429U and K15432U) were used for measuring serum IgG antibody levels. For total IgG binding, serum was diluted 1:1,000 in kit Dilution 100 buffer (MSD, Cat# R50AA). A SULFO-TAG anti-human IgG antibody (included in MSD kit Cat# K15429U) was used for signal detection. Results were reported as AU/ml using a human serum-based reference standard. For sVNT assays, serum was diluted 1:100 or 1:200 in the kit Dilution 100 buffer (MSD, Cat# R50AA), and results were reported as ACE2-binding percent inhibition using the following formula: 1- (average sample signal/average Diluent 100 only signal) ×100. ACE2 Calibration Reagent (included in the MSD kit) was used a positive control, showing 100% inhibition.

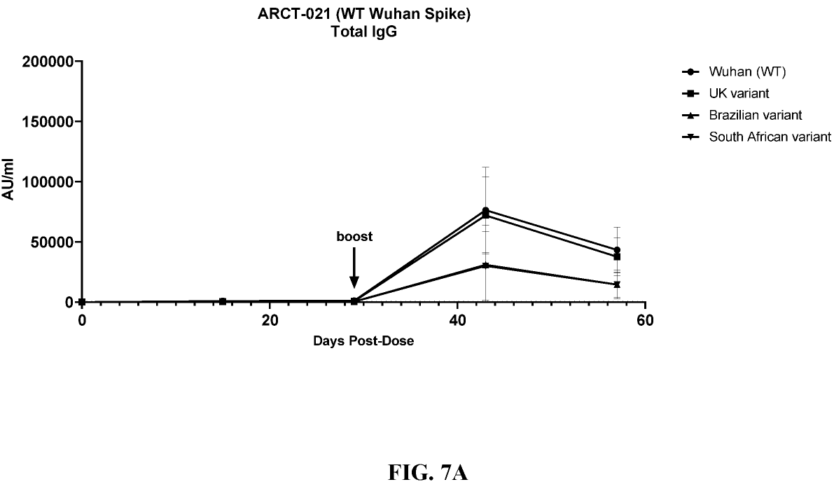

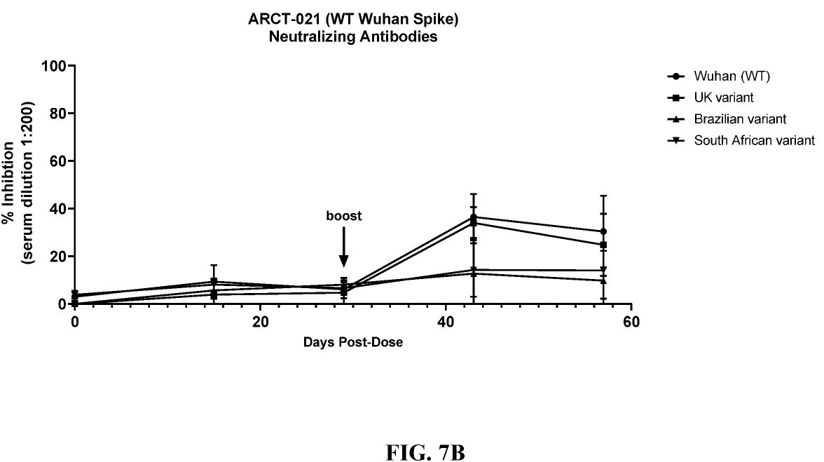

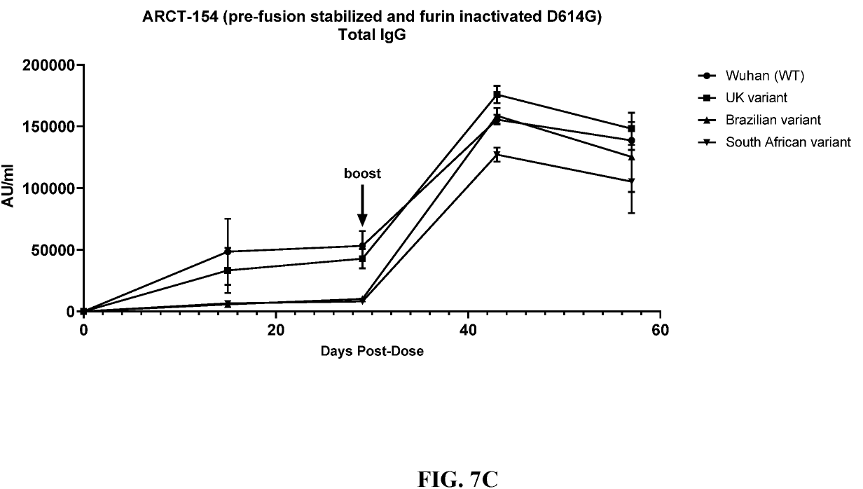

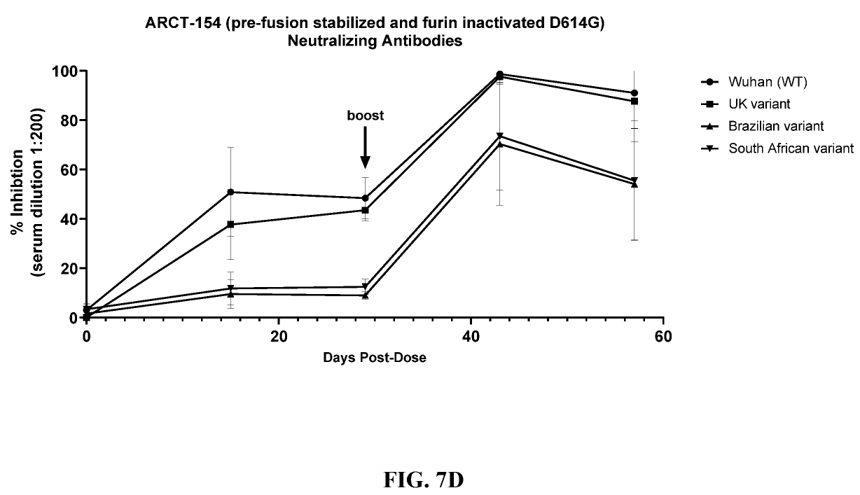

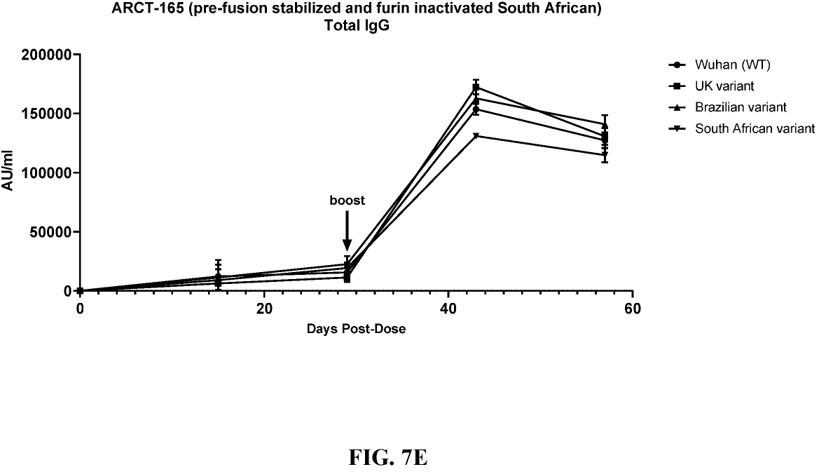

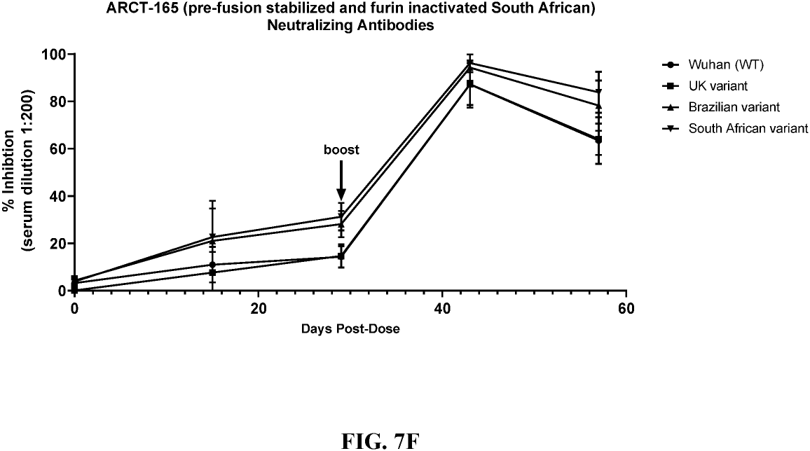

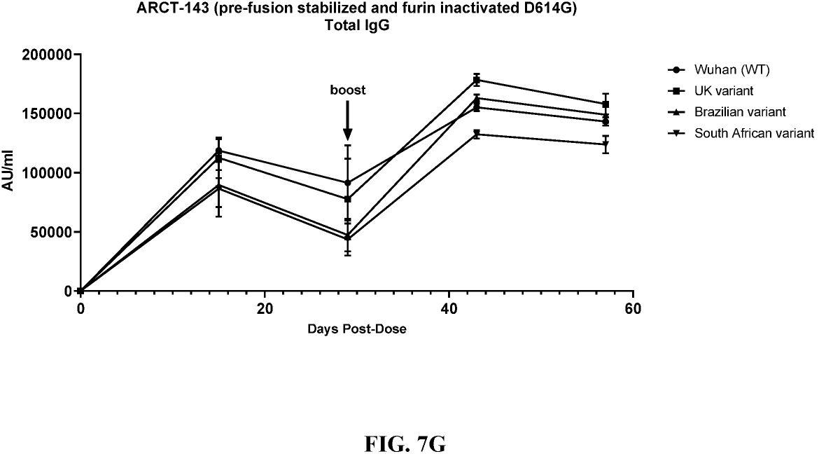

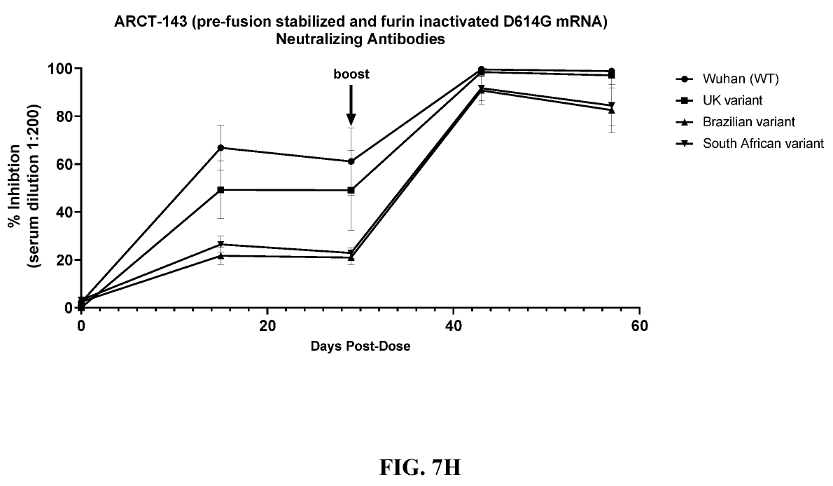

Results for total IgG and neutralizing antibodies upon immunization with lipid-formulated self-replicating RNA encoding a wild-type (Wuhan) SARS-CoV-2 spike glycoprotein (SEQ ID NO:18; ARCT-021 / mARM3015.5), a SARS-CoV-2 D614 variant spike glycoprotein (SEQ ID NO:2; ARCT-154 / mARM3280), or a SARS-CoV-2 South Africa variant spike glycoprotein (SEQ ID NO:1; ARCT-165 / mARM3325) are shown in FIGS. 7A-F. Results for total IgG and neutralizing antibodies upon immunization with lipid-formulated mRNA encoding a SARS-CoV-2 D614 variant spike glycoprotein (SEQ ID NO: 32; ARCT-143 / mARM3290) are shown in FIGS. 7G-H.

Immunization of NHPs with lipid-formulated self-replicating RNA encoding a wild-type (Wuhan) SARS-CoV-2 spike glycoprotein (SEQ ID NO:18; ARCT-021 / mARM3015.5; FIGS. 7A-B), a SARS-CoV-2 D614 variant spike glycoprotein (SEQ ID NO:2; ARCT-154 / mARM3280; FIGS. 7C-D), or a SARS-CoV-2 South Africa variant spike glycoprotein (SEQ ID NO:1; ARCT-165 / mARM3325; FIGS. 7E-F) elicited a SARS-CoV-2 specific IgG and neutralizing antibody responses against both wild-type and variant SARS-CoV-2 glycoproteins, including Wuhan (wild-type), UK (B. 1.1.7; Alpha), Brazil (P1; Gamma), and South Africa (B. 1.351; Beta) variants. Greater IgG and neutralizing antibody responses to both wild-type and variant SARS-CoV-2 glycoproteins were seen upon immunization with lipid-formulated self-replicating RNA encoding a SARS-CoV-2 D614 variant spike glycoprotein (SEQ ID NO:2; ARCT-154 / mARM3280; FIGS. 7C-D) or a SARS-CoV-2 South Africa variant glycoprotein (SEQ ID NO:1; ARCT-165 / mARM3325; FIGS. 7E-F) as compared to immunization with lipid-formulated self-replicating RNA encoding a wild-type (Wuhan) SARS-CoV-2 spike glycoprotein (SEQ ID NO:18; ARCT-021 / mARM3015.5; FIGS. 7A-B).

Immunization of NHPs with lipid-formulated mRNA encoding a SARS-CoV-2 D614 variant spike glycoprotein (SEQ ID NO: 32; ARCT-143 / mARM3290) also resulted in higher specific IgG levels against both wild-type and different SARS-CoV-2 variant spike glycoproteins as compared to immunization with self-replicating RNA encoding a wild-type (Wuhan) SARS-CoV-2 spike glycoprotein (SEQ ID NO:18; ARCT-021 / mARM3015.5 (FIG. 7A; FIG. 7G). Neutralizing antibody levels upon immunization with mRNA encoding SARS-CoV-2 D614G spike glycoprotein were likewise greater as compared to neutralizing antibody levels seen upon immunization with self-replicating RNA encoding wild-type (Wuhan) SARS-CoV-2 spike glycoprotein (FIG. 7B; FIG. 7H).

These results show that immunization of NHPs with self-replicating RNA or mRNA encoding SARS-CoV-2 variant D614G or variant South Africa spike glycoproteins elicits effective humoral immune responses, including neutralizing antibodies that were effective against wild-type and numerous SARS-CoV-2 variant glycoproteins.

### EXAMPLE 5

This example describes immunogenicity of influenza hemagglutinin (HA) expressed from self-replicating RNA or mRNA.

Self-replicating RNA and mRNA vaccine constructs were designed to encode the full-length hemagglutinin (HA) protein from influenza virus A/California/07/2009 (H1N1) (HA amino acid sequence: SEQ ID NOs: 47 and 53 for self-replicating RNA and mRNA, respectively; nucleic acid sequence: SEQ ID NOs: 46 and 52 for self-replicating RNA and mRNA, respectively). As described above for Example 1, the mRNA vaccine construct encoding HA included a tobacco etch virus (TEV) 5′ UTR (SEQ ID NO:49) and a Xenopus beta-globin (Xbg) 3′ UTR (SEQ ID NO:50 (without poly-A tail); SEQ ID NO:52 (with poly-A tail)). Both self-replicating RNA (SEQ ID NO:40; entire RNA mARM3124) and mRNA (SEQ ID NO:48; entire RNA sequence mARM3038) vaccine constructs were encapsulated in the same lipid nanoparticle (LNP) composition that included four lipid excipients (an ionizable cationic lipid, 1,2-distearoyl-sn-glycero-3-phosphocholine (DSPC), cholesterol, and PEG2000-DMG) dispersed in HEPES buffer (pH 8.0) containing sodium chloride and the cryoprotectants sucrose and glycerol. The N:P ratio of complexing lipid and RNA was approximately 9:1. The ionizable cationic lipid had the following structure:

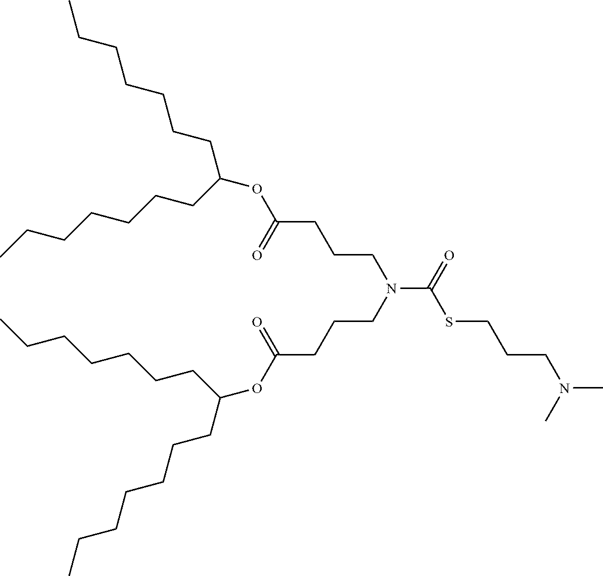 [SMILES: CCCCCCCC(CCCCCCC)OC(=O)CCCN(CCCC(=O)OC(CCCCCCC)CCCCCCC)C(=O)SCCCN(C)C]

Five female, 8-10 week old Balb/c mice were injected intramuscularly with 2 µg of mRNA or self-replicating RNA encoding HA. Mice were bled on days 14, 28, 42, and 56, followed by hemagglutination inhibition (HAI) assay using serially diluted sera. The reciprocal of the highest dilution of serum that caused inhibition of hemagglutination was considered the HAI titer, with a titer of 1 /40 being protective against influenza virus infection and four-fold higher titers than baseline indicating seroconversion.

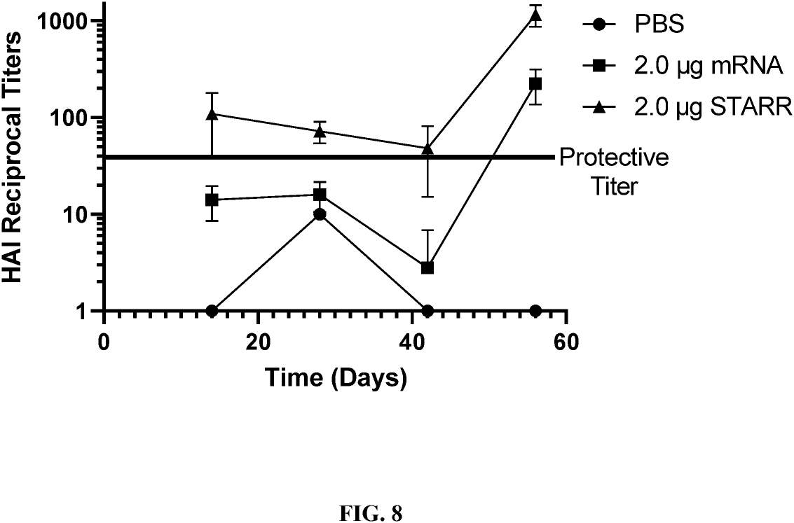

Results in FIG. 8 show that protective HAI titers were obtained with self-replicating RNA and mRNA encoding HA. HAI titers for the self-replicating RNA construct encoding HA were greater than HAI titers for the mRNA encoding HA at all time points. In addition, protective HAI titers were seen for the self-replicating RNA construct encoding HA beginning at day 14 that were maintained at least until day 56. mRNA encoding HA showed protective HAI titers at day 56.

These results show that both the self-replicating RNA and mRNA constructs encoding HA elicited protective HA antibody titers, with self-replicating RNA eliciting protective HAI titers earlier after immunization as compared to mRNA.

### EXAMPLE 6

### Lyophilization of Self-Replicating RNA-Lipid Nanoparticle Formulation Materials and Methods Generally

The processes performed in this example were conducted using lipid nanoparticle compositions that were manufactured according to well-known processes, for example, those described in U.S. App. No. 16/823,212, the contents of which are incorporated by reference for the specific purpose of teaching lipid nanoparticle manufacturing processes. The lipid nanoparticle compositions and the lyophilized products were characterized for several properties. The materials and methods for these characterization processes as well as a general method of manufacturing the lipid nanoparticle compositions that were used for lyophilization experiments are provided in this example.

### Lipid Nanoparticle Manufacture

Lipid nanoparticle formulations used in this example were manufactured by mixing lipids (ionizable cationic lipid (ATX-126): helper lipid: cholesterol: PEG-lipid) in ethanol with RNA dissolved in citrate buffer. The mixed material was instantaneously diluted with Phosphate Buffer. Ethanol was removed by dialysis against phosphate buffer using regenerated cellulose membrane (100 kD MWCO) or by tangential flow filtration (TFF) using modified polyethersulfone (mPES) hollow fiber membranes (100 kD MWCO). Once the ethanol was completely removed, the buffer was exchanged with HEPES (4-(2-hydroxyethyl)-1-piperazineethanesulfonic acid) buffer containing 10-300 (for example, 40-60) mM NaCl and 5-15% sucrose, pH 7.3. The formulation was concentrated followed by 0.2 µm filtration using PES filters. The RNA concentration in the formulation was then measured by RiboGreen fluorimetric assay, and the concentration was adjusted to a final desired concentration by diluting with HEPES buffer containing 10-100 (for example 40-60) mM NaCl, 0-15% sucrose, pH 7.2-8.5 containing glycerol. If not used immediately for further studies, the final formulation was then filtered through a 0.2 µm filter and filled into glass vials, stoppered, capped and placed at -70 ± 5° C. The lipid nanoparticles formulations were characterized for their pH and osmolality. Lipid content and RNA content were measured by high performance liquid chromatography (HPLC), and mRNA integrity by was measured by fragment analyzer. Dynamic Light Scattering (DLS)

The average particle size (z) and polydispersity index (PDI) of lipid nanoparticle formulations used in the Examples was measured by dynamic light scattering on a Malvern Zetasizer Nano ZS (United Kingdom).

### RiboGreen Assay

The encapsulation efficiency of the lipid nanoparticle formulations was characterized using the RiboGreen fluorometric assay. RiboGreen is a proprietary fluorescent dye (Molecular Probes/Invitrogen a division of Life Technologies, now part of Thermo Fisher Scientific of Eugene, Oregon, United States) that is used in the detection and quantification of nucleic acids, including both RNA and DNA. In its free form, RiboGreen exhibits little fluorescence and possesses a negligible absorbance signature. When bound to nucleic acids, the dye fluoresces with an intensity that is several orders of magnitude greater than the unbound form. The fluorescence can then be detected by a sensor (fluorimeter) and the nucleic acid can be quantified.

### Lyophilization Process

Self-Replicating RNAs (aka Replicon RNA) are typically larger than the average mRNA, and tests were designed to determine whether self-replicating RNA lipid nanoparticle formulations could be successfully lyophilized. The quality of lyophilized lipid nanoparticle formulations was assessed by analyzing the formulations post-lyophilization and comparing this to the lipid nanoparticle formulation prior to lyophilization as well as after a conventional freeze/thaw cycle (i.e., frozen at ~ -70° C. then allowed to thaw at room temperature).

The analysis of the lipid nanoparticle formulations included the analysis of particle size and polydispersity (PDI) and encapsulation efficiency (%Encap). The particle size post-lyophilization was compared to the particle size pre-lyophilization and the difference can be reported as a delta (δ). The various compositions tested were screened as to whether a threshold of properties was met including minimal particle size increase (δ < 10 nm), the maintenance of PDI (< 0.2), and maintenance of high encapsulation efficiency (> 85%).

The lipid nanoparticle formulations were prepared as described above, with self-replicating RNA (SEQ ID NO: 18). The resulting lipid nanoparticle formulation was then processed with a buffer exchange to form a prelyophilization suspension having a concentration of 0.05 to 2.0 mg/mL self-replicating RNA, 0.01 to 0.05 M potassium sorbate, 0.01 to 0.10 % w/v Poloxamer 188 (Kolliphor®), 14 to 18% w/v sucrose, 25 to 75 mM NaCl and 15 to 25 mM pH 8.0 Tris buffer. The prelyophilization formulation was then lyophilized in a Millrock Revo Freeze Dryer (Model No. RV85S4), using aliquots of 2.0 mL of suspension and the lyophilization cycle provided in Table 18 below.

TABLE 18

Lyophilization Cycle for Self-Replicating RNA-Lipid Nanoparticle Formulation
Freeze drying cycle

| Step | shelf temperature | step duration | chamber vacuum |
|---|---|---|---|
| | (°C, ±2° C.) | (h:min) | (mbar) |
| Initial Freezing | - 50 | 4:00 | atmosphere |
| Evacuation | -50 | 00:30 - 01:45 | from atmosph. pressure to 0.05 |
| Primary drying (ramp down) | - 50 → 0 | 63:00 | 0.05 |
| Secondary drying (ramp up) | 0 → +25 | 39:30 | 0.05 |
| Backfill with N₂and stoppering | 25 | 00:10 - 00:20 | 700 ± 50 |
| Aeration with air | 5 | 00:10 - 00:20 | atmosphere |

The lyophilized particles prepared following the methods described above were reconstituted in 2 mL of water and characterized using DLS and RiboGreen. The results provided in Table 19 below show that the lyophilized compositions were found to produce lyophilized lipid nanoparticle formulations with adequate size, polydispersity, and delta values (~5.3 nm) upon reconstitution.

TABLE 19

Self-Replicating RNA-Lipid Nanoparticle Characteristics Pre- and Post-LYO

| | Average Particle Size (nm) | PDI | encap (%) |
|---|---|---|---|
| Pre-LYO | 76.3 | 0.129 | 97 |
| Post-LYO | 81.6 | 0.152 | 93 |

Any self-replicating RNA and any mRNA can be prepared as a lyophilized formulation using the processes described above, including any self-replicating RNA and any mRNA delivering antigenic proteins provided herein. Furthermore, lyophilized formulations can be administered to induce immune responses to encoded antigenic proteins, such as SARS-CoV-2 spike glycoproteins and variants thereof.

### EXAMPLE 7

This example describes immunogenicity of liquid and lyophilized self-replicating RNA formulations.

Immunogenicity of self-replicating RNA (SEQ ID NO: 18) formulated as a lyophilized lipid nanoparticle (LYO-LNP) was tested in BALB/c mice in two separate preclinical studies and compared with the liquid (frozen) LNP formulation (Liquid-LNP). Each study included the use of a PBS dosing group as a negative control and a Liquid dosing group (Liquid-LNP) as a positive control. Both LYO-LNP and Liquid-LNP formulations were dosed at 0.2 and 2 µg. There were n=5 animals per dose group in each study. Test formulations were administered intramuscularly (IM) and serum was collected at various timepoints (Days 10, 19, 31 for the first study and Days 10, 20, 30 for the second study) post-immunization to measure the production of anti-SARS-CoV-2 spike protein IgG using a Luminex bead fluorescent assay.

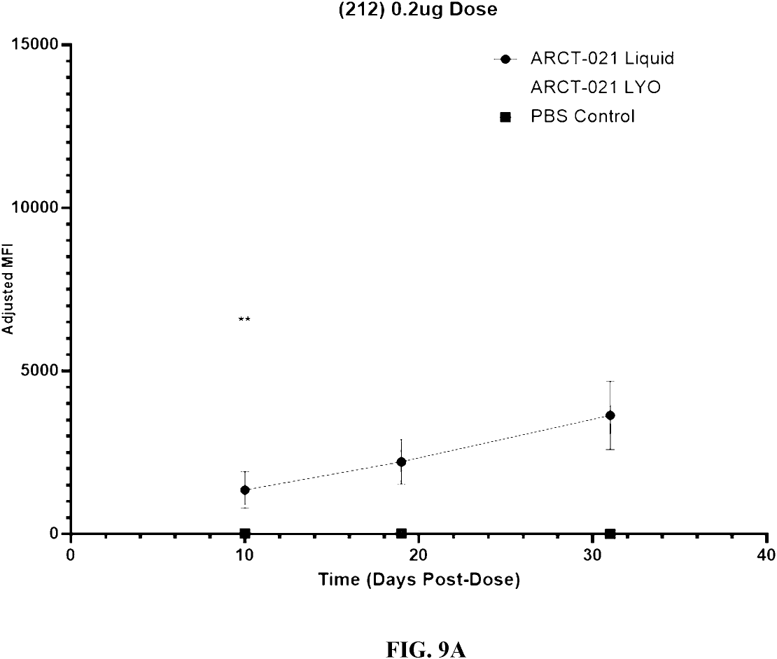

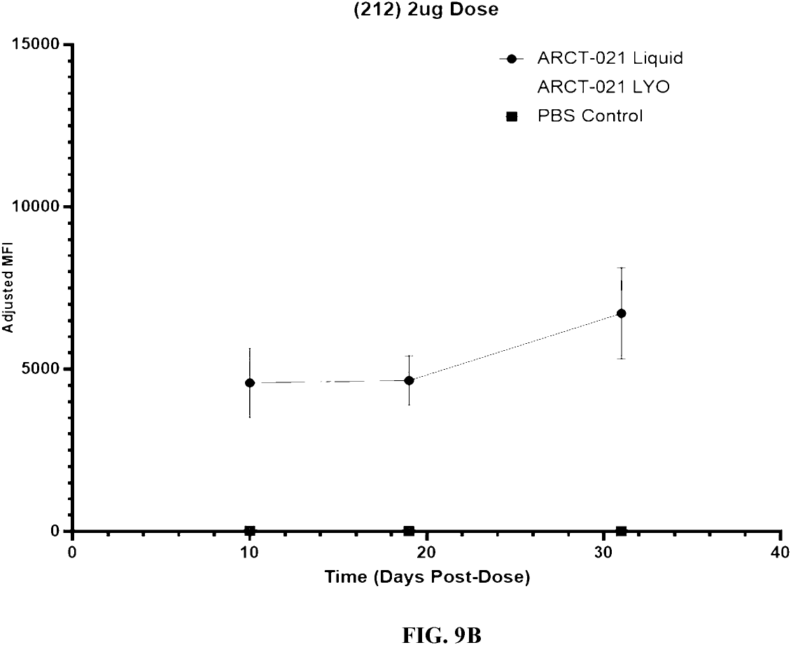

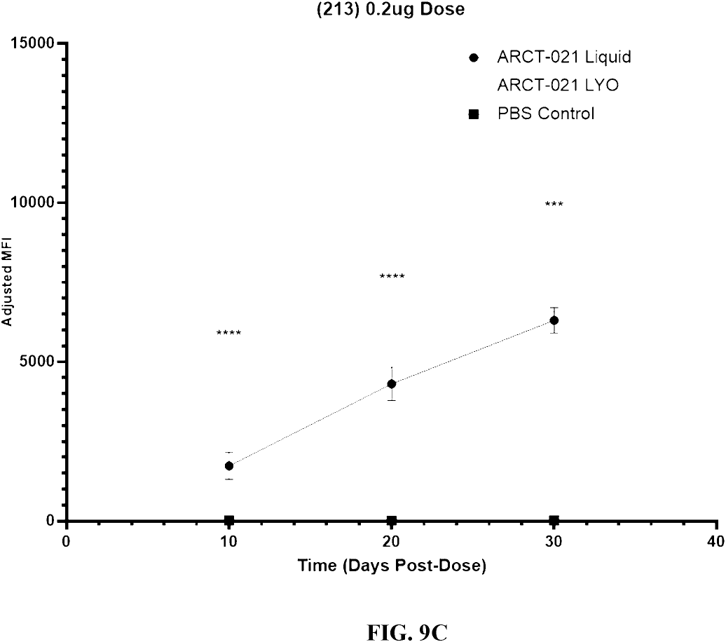

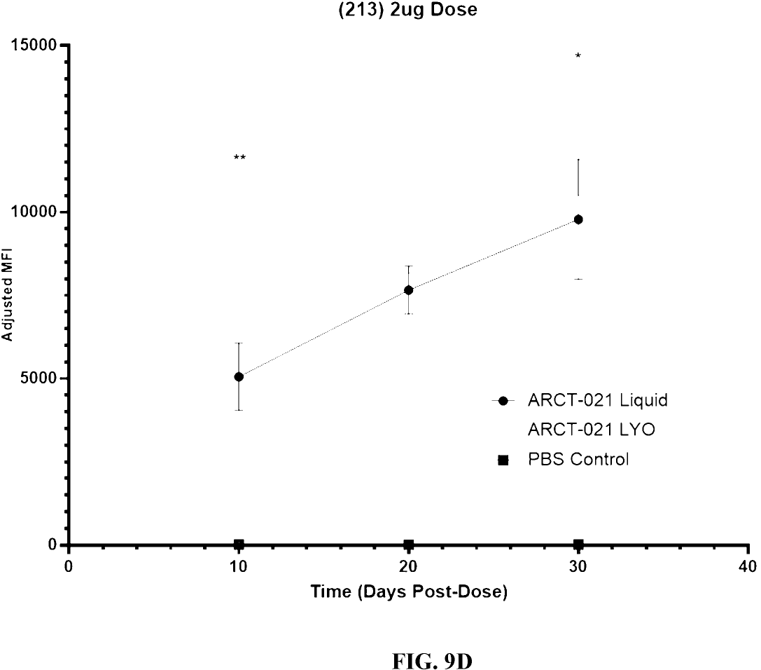

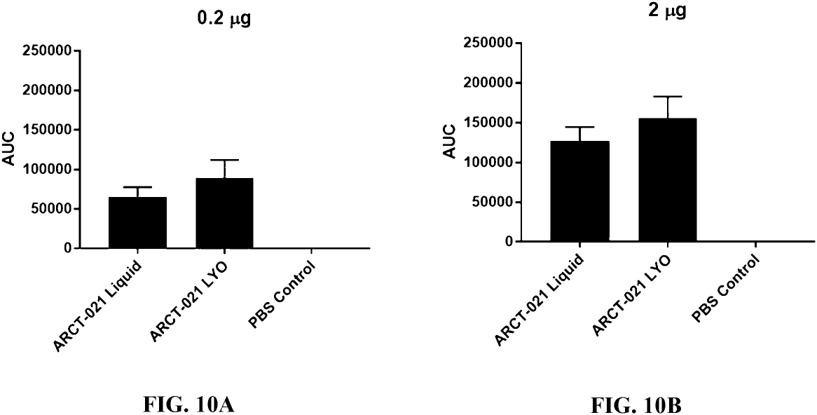

In both studies, anti-SARS-CoV-2 spike protein IgGs were detected in serum in a time- and dose-dependent manner for both Liquid-LNP and LYO-LNP formulations, whereas PBS injection did not elicit an immunogenic response (FIGS. 9A-9D). There was no statistical difference in immunogenicity seen between Liquid-LNP and LYO-LNP dose groups in the first study, whereas LYO-LNP produced statistically different and greater IgG than Liquid-LNP in the second study. Without being limited by theory, under-powering (n=5/group) of these two separate studies may have contributed to the statistical differences in immunogenicity results observed in the two studies. In combining the results of both studies, no statistically significant differences were observed between Liquid-LNP and LYO-LNP formulations at the 0.2 and 2 µg dose levels (FIGS. 10A, 10B). Taken together, the results of these studies demonstrate that the immunogenicity of the liquid and lyophilized formulations were comparable.

In summary, the liquid and lyophilized formulations of a self-replicating RNA vaccine (SEQ ID NO: 18) showed comparable immunogenicity. The vaccine can induce effective, adaptive humoral (neutralizing antibodies) and cellular (CD8+) immune responses targeting the SARS-CoV-2 spike (S) glycoprotein. The vaccine also elicited induction of anti-spike glycoprotein antibodies (IgG) levels that were higher than those seen for a conventional mRNA vaccine and induced production of IgG antibodies at a faster rate than a conventional mRNA vaccine. It continued to elicit increasing levels of IgG up to 50 days post vaccination whereas the conventional mRNA vaccine plateaued by day 10 post vaccination. It produced an RNA dose-dependent increase in CD8+ T lymphocytes and a balanced, Th1 dominant CD4+ T helper cell immune response with no skew towards a Th2 response.

### SEQUENCES

SEQ ID NO:1 - mARM3325 (South Africa B.1.351)

atgggcggcgcatgagagaagcccagaccaattacctacccaaaatggag
aaagttcacgttgacatcgaggaagacagcccattcctcagagctttgca
gcggagcttcccgcagtttgaggtagaagccaagcaggtcactgataatg
accatgctaatgccagagcgttttcgcatctggcttcaaaactgatcgaa
acggaggtggacccatccgacacgatccttgacattggaagtgcgcccgc
ccgcagaatGTATTCTAAGCACAAGTATCATTGTATCtgtccgatgagat
gtgcggaagatccggacagattgtataagtatgcaactaagctgaagaaa
aactgtaaggaaataactgataaggaattggacaagaaaatgaaggagct
ggccgccgtcatgagcgaccctgacctggaaactgagactatgtgcctcc
acgacgacgagtcgtgtcgctacgaagggcaagtcgctgtttaccaggat
gtatacgcCGTcGACGGCCCCACCAGCCTGTACCACCAGGCCAACAAGGG
CGTGAGGGTGGCCTACTGGATCGGCTTCGACACCACACCCTTCATGTTCA
AGAACCTGGCCGGCGCCTACCCCAGCTACAGCACCAACTGGGCCGACGAG
ACAGTGCTGACCGCCAGGAACATCGGCCTGTGCAGCAGCGACGTGATGGA
GAGGAGCCGGAGGGGCATGAGCATCCTGAGGAAGAAGTACCTGAAGCCCA
GCAACAACGTGCTGTTCAGCGTGGGCAGCACCATCTACCACGAGAAGAGG
GACCTGCTGAGGAGCTGGCACCTGCCCAGCGTGTTCCACCTGAGGGGCAA
GCAGAACTACACCTGCAGGTGCGAGACAATCGTGAGCTGCGACGGCTACG
TGGTGAAGAGGATCGCCATCAGCCCCGGCCTGTACGGCAAGCCCAGCGGC
TACGCCGCCACCATGCACAGGGAGGGCTTCCTGTGCTGCAAGGTGACCGA
CACCCTGAACGGCGAGAGGGTGAGCTTCCCCGTGTGCACCTACGTGCCCG
CCACCCTGTGCGACCAGATGACCGGCATCCTGGCCACCGACGTGAGCGCC
GACGACGCCCAGAAGCTGCTGGTGGGCCTGAACCAGAGGATCGTGGTGAA
CGGCAGGACCCAGAGGAACACCAACACCATGAAGAACTACCTGCTGCCCG
TGGTGGCCCAGGCCTTCGCCAGGTGGGCCAAGGAGTACAAGGAGGACCAG
GAGGACGAGAGGCCCCTGGGCCTGAGGGACcGaCAGCTGGTGATGGGCTG
CTGCTGGGCCTTCAGGCGGCACAAGATCACCAGCATCTACAAGAGGCCCG
ACACCCAGACCATCATCAAGGTGAACAGCGACTTCCACAGCTTCGTGCTG
CCCAGGATCGGCAGCAACACCCTGGAGATCGGCCTGAGGACCCGGATCAG
GAAGATGCTGGAGGAGCACAAGGAGCCCAGCCCTCTGATCACCGCCGAGG
ACGTGCAGGAGGCCAAGTGCGCCGCCGACGAGGCCAAGGAGGTGAGGGAG
GCCGAGGAGCTGAGGGCCGCCCTGCCTCCCCTGGCCGCCGACGTGGAGGA
GCCCACCCTGGAGGCCGACGTGGACCTGATGCTGCAGGAGGCCGGCGCCG
GCAGCGTGGAGACACCCAGGGGCCTGATCAAGGTGACCAGCTACGACGGC
GAGGACAAGATCGGCAGCTACGCCGTGCTcAGCCCTCAGGCCGTGCTGAA
GTCCGAGAAGCTGAGCTGCATCCACCCTCTGGCCGAGCAGGTGATCGTGA
TCACCCACAGCGGCAGGAAGGGCAGGTACGCCGTGGAGCCCTACCACGGC
AAGGTGGTGGTCCCCGAGGGCCACGCCATCCCCGTGCAGGACTTCCAGGC
CCTGAGCGAGAGCGCCACCATCGTGTATAACGAGAGGGAGTTCGTGAACA
GGTACCTGCACCACATCGCCACCCACGGCGGCGCCCTGAACACCGACGAG
GAGTACTACAAGACCGTGAAGCCCAGCGAGCACGACGGCGAGTACCTGTA
CGACATCGACAGGAAGCAGTGCGTGAAGAAGGAGCTGGTGACCGGCCTGG
GCCTGACCGGCGAGCTGGTGGACCCTCCCTTCCACGAGTTCGCCTACGAG
AGCCTGAGGACCAGGCCCGCCGCTCCCTACCAGGTGCCCACCATCGGCGT
GTACGGCGTGCCCGGCAGCGGCAAGAGCGGCATCATCAAGAGCGCCGTGA
CCAAGAAGGACCTGGTGGTGAGCGCCAAGAAGGAGAACTGCGCCGAGATC
ATCAGGGACGTGAAGAAGATGAAGGGCCTGGACGTGAACGCCAGGACCGT
GGACAGCGTGCTcCTGAACGGCTGCAAGCACCCCGTGGAGACACTGTATA
TCGACGAGGCCTTCGCCTGCCACGCCGGCACCCTGAGGGCCCTGATCGCC
ATCATCAGGCCCAAGAAGGCCGTGCTGTGCGGCGACCCCAAGCAGTGCGG
CTTCTTCAACATGATGTGCCTGAAGGTGCACTTCAACCACGAGATCTGCA
CCCAGGTGTTCACAAGAGCATCAGCAGGCGGTGCACCAAGAGCGTGACCA
GCGTGGTGAGCACCCTGTTCTACGACAAGAAGATGAGGACCACCAACCCC
AAGGAGACAAAGATCGTGATCGACACCACCGGCAGCACCAAGCCCAAGCA
GGACGACCTGATCCTGACCTGCTTCAGGGGCTGGGTGAAGCAGCTGCAGA
TCGACTACAAGGGCAACGAGATCATGACCGCCGCCGCTAGCCAGGGCCTG
ACCAGGAAGGGCGTGTACGCCGTGAGGTACAAGGTGAACGAGAATCCCCT
GTACGCCCCTACCAGCGAGCACGTGAACGTcCTGCTGACCAGGACCGAGG
ACAGGATCGTGTGGAAGACCCTGGCCGGCGACCCCTGGATCAAGACCCTG
ACCGCCAAGTACCCCGGCAACTTCACCGCCACCATCGAGGAGTGGCAGGC
CGAGCACGACGCCATCATGAGGCACATCCTGGAGAGGCCCGACCCCACCG
ACGTGTTCCAGAACAAGGCCAACGTGTGCTGGGCCAAGGCCCTGGTGCCC
GTGCTGAAGACCGCCGGCATCGACATGACCACCGAGCAGTGGAACACCGT
GGACTACTTCGAGACAGACAAGGCCCACAGCGCCGAGATCGTGCTGAACC
AGCTGTGCGTGAGGTTCTTCGGCCTGGACCTGGACAGCGGCCTGTTCAGC
GCCCCTACCGTGCCCCTGAGCATCAGGAACAACCACTGGGACAACAGCCC
CAGCCCCAACATGTACGGCCTGAACAAGGAGGTGGTGAGGCAGCTGAGCA
GGCGGTACCCTCAGCTGCCCAGGGCCGTGGCCACCGGCAGGGTGTACGAC
ATGAACACCGGCACCCTGAGGAACTACGACCCCAGGATCAACCTGGTGCC
CGTGAACAGGCGGCTGCCaCACGCCCTGGTGCTGCACCACAACGAGCACC
CTCAGAGCGACTTCAGCAGCTTCGTGAGCAAGCTGAAGGGCAGGACCGTG
CTGGTGGTGGGCGAGAAGCTGAGCGTGCCCGGCAAGATGGTGGACTGGCT
GAGCGACAGGCCCGAGGCCACCTTCCGGGCCAGGCTGGACCTGGGCATCC
CCGGCGACGTGCCCAAGTACGACATCATCTTCGTGAACGTGAGGACCCCT
TACAAGTACCACCACTACCAGCAGTGCGAGGACCACGCCATCAAGCTGAG
CATGCTGACCAAGAAGGCCTGCCTGCACCTGAACCCCGGCGGCACCTGCG
TGAGCATCGGCTACGGCTACGCCGACAGGGCCAGCGAGAGCATCATCGGC
GCCATCGCCAGGCTGTTCAAGTTCAGCAGGGTGTGCAAGCCCAAGAGCAG
CCTGGAGGAGACAGAGGTGCTGTTCGTGTTCATCGGCTACGACCGGAAGG
CCAGGACCCACAACCCCTACAAGCTGAGCAGCACCCTGACCAACATCTAC
ACCGGCAGCAGGCTGCACGAGGCCGGCTGCGCCCCTAGCTACCACGTGGT
GAGGGGCGACATCGCCACCGCCACCGAGGGCGTGATCATCAACGCCGCCA
ACAGCAAGGGCCAGCCCGGCGGCGGGGTGTGCGGCGCCCTGTATAAGAAG
TTCCCCGAGAGCTTCGACCTGCAGCCCATCGAGGTGGGCAAGGCCAGGCT
GGTGAAGGGCGCCGCCAAGCACATCATCCACGCCGTGGGCCCCAACTTCA
ACAAGGTGAGCGAGGTGGAGGGCGACAAGCAGCTGGCCGAGGCCTACGAG
AGCATCGCCAAGATCGTGAACGACAACAACTACAAGAGCGTGGCCATCCC
TCTGCTGAGCACCGGCATCTTCAGCGGCAACAAGGACAGGCTGACCCAGA
GCCTGAACCACCTGCTGACCGCCCTGGACACCACCGACGCCGACGTGGCC
ATCTACTGCAGGGACAAGAAGTGGGAGATGACCCTGAAGGAGGCCGTGGC
CAGGCGGGAGGCCGTGGAGGAGATCTGCATCAGCGACGACAGCAGCGTGA
CgGAGCCCGACGCCGAGCTGGTGAGGGTGCACCCCAAGAGCAGCCTGGCC
GGCAGGAAGGGCTACAGCACCAGCGACGGCAAGACCTTCAGCTACCTGGA
GGGCACCAAGTTCCACCAGGCCGCCAAGGACATCGCCGAGATCAACGCCA
TGTGGCCCGTGGCCACCGAGGCCAACGAGCAGGTGTGCATGTATATCCTG
GGCGAGAGCATGAGCAGCATCAGGAGCAAGTGCCCCGTGGAGGAGAGCGA
GGCCAGCACCCCTCCCAGCACCCTGCCCTGCCTGTGCATCCACGCCATGA
CCCCTGAGAGGGTGCAGCGGCTGAAGGCCAGCAGGCCCGAGCAGATCACC
GTGTGCAGCAGCTTCCCTCTGCCCAAGTACcGGATCACCGGCGTGCAGAA
GATCCAGTGCAGCCAGCCCATCCTGTTCAGCCCCAAGGTGCCCGCCTACA
TCCACCCCAGGAAGTACCTGGTGGAGACACCCCCCGTGGACGAGACACCC
GAGCCCAGCGCCGAGAACCAGAGCACCGAGGGCACCCCTGAGCAGCCTCC
CCTGATCACCGAGGACGAGACAAGGACCAGGACgCCcGAGCCCATCATCA
TTGAGGAGGAAGAGGAGGACAGCATCAGCCTGCTGAGCGACGGCCCCACC
CACCAGGTGCTGCAGGTGGAGGCCGACATCCACGGCCCTCCCAGCGTGAG
CAGCTCCAGCTGGAGCATCCCTCACGCCAGCGACTTCGACGTGGACAGCC
TGAGCATCCTGGACACCCTGGAGGGCGCCAGCGTGACCAGCGGCGCCACC
AGCGCCGAGACAAACAGCTACTTCGCCAAGAGCATGGAGTTCCTGGCCAG
GCCCGTGCCCGCCCCTAGGACCGTGTTCAGGAACCCTCCCCACCCCGCCC
CTAGGACCAGGACCCCTAGCCTGGCCCCTAGCAGGGCCTGCAGCAGGACC
AGCCTGGTGAGCACCCCTCCCGGCGTGAACcGGGTGATCACCAGGGAGGA
GCTGGAGGCCCTGACCCCTAGCAGGACCCCTAGCAGGAGCGTGAGCAGGA
CCAGCCTGGTGAGCAACCCTCCCGGCGTGAACcGGGTGATCACCAGGGAG
GAGTTCGAGGCCTTCGTGGCCCAGCAGCAAAGGCGGTTCGACGCCGGCGC
CTACATCTTCAGCAGCGACACCGGCCAGGGCCACCTGCAGCAGAAGTCCG
TGAGGCAGACCGTGCTGAGCGAGGTGGTcCTGGAGAGGACgGAGCTGGAG
ATCAGCTACGCCCCTAGGCTGGACCAGGAGAAGGAGGAGCTGCTGAGGAA
GAAGCTGCAGCTGAACCCCACCCCTGCCAACAGGAGCAGGTACCAGAGCA
GGAAGGTGGAGAACATGAAGGCCATCACCGCCAGGCGGATCCTGCAGGGC
CTGGGCCACTACCTGAAGGCCGAGGGCAAGGTGGAGTGCTACAGGACCCT
GCACCCCGTGCCCCTGTACTCCAGCTCCGTGAACAGGGCCTTCAGCAGCC
CCAAGGTGGCCGTGGAGGCCTGCAACGCCATGCTGAAGGAGAACTTCCCC
ACCGTGGCCAGCTACTGCATCATCCCCGAGTACGACGCCTACCTGGACAT
GGTGGACGGCGCCAGCTGCTGCCTGGACACCGCCAGCTTCTGCCCCGCCA
AGCTGAGGAGCTTCCCCAAGAAGCACAGCTACCTGGAGCCCACCATCAGG
AGCGCCGTGCCCAGCGCCATCCAGAACACCCTGCAGAACGTGCTGGCCGC
CGCTACCAAGAGGAACTGCAACGTGACCCAGATGAGGGAGCTGCCCGTGC
TGGACAGCGCCGCCTTCAACGTGGAGTGCTTCAAGAAGTACGCCTGCAAC
AACGAGTACTGGGAGACATTCAAGGAGAACCCCATCAGGCTGACCGAGGA
GAACGTGGTGAACTACATCACCAAGCTGAAGGGCCCCAAGGCCGCCGCTC
TGTTCGCCAAGACCCACAACCTGAACATGCTcCAGGACATCCCTATGGAC
AGGTTCGTGATGGACCTGAAGAGGGACGTGAAGGTGACCCCTGGCACCAA
GCACACCGAGGAGAGGCCCAAGGTGCAGGTGATCCAGGCCGCCGACCCTC
TGGCCACCGCCTACCTGTGCGGCATCCACAGGGAGCTGGTGAGGCGGCTG
AACGCCGTcCTGCTGCCCAACATCCACACCCTGTTCGACATGAGCGCCGA
GGACTTCGACGCCATCATCGCCGAGCACTTCCAGCCCGGCGACTGCGTGC
TGGAGACAGACATCGCCAGCTTCGACAAGAGCGAGGACGACGCTATGGCC
CTGACCGCCCTGATGATCCTGGAGGACCTGGGCGTGGACGCCGAGCTGCT
GACCCTGATCGAGGCCGCCTTCGGCGAGATCAGCAGCATCCACCTGCCCA
CCAAGACCAAGTTCAAGTTCGGCGCCATGATGAAGTCCGGCATGTTCCTG
ACCCTGTTCGTGAACACCGTGATCAACATCGTGATCGCCAGCAGGGTGCT
GCGGGAGAGGCTGACCGGCAGCCCCTGCGCCGCCTTCATCGGCGACGACA
ACATCGTGAAGGGCGTGAAGTCCGACAAGCTGATGGCCGACAGGTGCGCC
ACCTGGCTGAACATGGAGGTGAAGATCATCGACGCCGTGGTGGGCGAGAA
GGCCCCTTACTTCTGCGGCGGCTTCATCCTGTGCGACAGCGTGACCGGCA
CCGCCTGCAGGGTGGCCGACCCTCTGAAGAGGCTGTTCAAGCTGGGCAAG
CCCCTGGCCGCCGACGACGAGCACGACGACGATAGGCGGAGGGCCCTGCA
CGAGGAGAGCACCAGGTGGAACcGGGTGGGCATCCTGAGCGAGCTGTGCA
AGGCCGTGGAGAGCAGGTACGAGACAGTGGGCACCAGCATCATCGTGATG
GCCATGACCACCCTGGCCAGCAGCGTcAAGTCCTTCAGCTACCTGAGGGG
GGCCCCTATAACTCTCTACGGCTAACCTGAATGGACTACGACATAGTCTA
GTCCGCCAAGGCCGCCACcATGTTCGTGTTCCTGGTGCTGCTGCCCCTGG
TGTCTAGCCAGTGCGTGAACCTGACCACCAGGACCCAGCTGCCTCCCGCC
TACACCAACAGCTTCACCAGGGGCGTGTACTACCCCGACAAGGTGTTCAG
GAGCAGCGTGCTGCACAGCACCCAGGACCTGTTCCTGCCCTTCTTCAGCA
ACGTGACCTGGTTCCACGCCATCCACGTGAGCGGCACCAACGGCACCAAG
AGGTTCGcCAACCCCGTGCTGCCCTTCAACGACGGCGTGTACTTCGCCAG
CACCGAGAAGTCCAACATCATCAGGGGCTGGATCTTCGGCACCACCCTGG
ACAGCAAGACCCAGAGCCTGCTGATCGTGAACAACGCCACCAACGTGGTG
ATCAAGGTGTGCGAGTTCCAGTTCTGCAACGACCCCTTCCTGGGCGTGTA
CTACCACAAGAACAACAAGAGCTGGATGGAGAGCGAGTTCAGGGTGTACT
CCAGCGCCAACAACTGCACCTTCGAGTACGTGAGCCAGCCCTTCCTGATG
GACCTGGAGGGCAAGCAGGGCAACTTCAAGAACCTGAGGGAGTTCGTGTT
CAAGAACATCGACGGCTACTTCAAGATCTACAGCAAGCCACCCCTATCAA
CCTGGTGAGGGgCCTGCCCCAGGGCTTCAGCGCCCTGGAGCCCCTGGTGG
ACCTGCCCATCGGCATCAACATCACCAGGTTCCAGACCCTGCTGGCCCTG
CACAGGAGCTACCTGACCCCTGGCGACAGCAGCTCCGGCTGGACCGCCGG
CGCCGCCGCTTACTACGTGGGCTACCTGCAGCCCAGGACCTTCCTGCTGA
AGTACAACGAGAACGGCACCATCACCGACGCCGTGGACTGCGCCCTGGAC
CCTCTGAGCGAGACAAAGTGCACCCTGAAGTCCTTCACCGTGGAGAAGGG
CATCTACCAGACCAGCAACTTCAGGGTGCAGCCCACCGAGAGCATCGTGA
GGTTCCCCAACATCACCAACCTGTGCCCCTTCGGCGAGGTGTTCAACGCC
ACCAGGTTCGCCAGCGTGTACGCCTGGAACAGGAAGAGGATCAGCAACTG
CGTGGCCGACTACAGCGTGCTGTATAACAGCGCCAGCTTCAGCACCTTCA
AGTGCTACGGCGTGAGCCCCACCAAGCTGAACGACCTGTGCTTCACCAAC
GTGTACGCCGACAGCTTCGTGATCAGGGGCGACGAGGTGAGGCAGATCGC
CCCTGGCCAGACCGGCAAcATCGCCGACTACAACTACAAGCTGCCCGACG
ACTTCACCGGCTGCGTGATCGCCTGGAACAGCAACAACCTGGACAGCAAG
GTGGGCGGCAACTACAACTACCTGTACCGGCTGTTCAGAAAGAGCAACCT
GAAGCCCTTCGAGAGGGACATCAGCACCGAGATCTACCAGGCCGGCAGCA
CCCCTTGCAACGGCGTGaAGGGCTTCAACTGCTACTTCCCTCTGCAGAGC
TACGGCTTCCAGCCCACCtACGGCGTGGGCTACCAGCCCTACAGGGTGGT
GGTCCTGAGCTTCGAGCTGCTGCACGCCCCTGCCACCGTGTGCGGCCCCA
AGAAGTCCACCAACCTGGTGAAGAACAAGTGCGTGAACTTCAACTTCAAC
GGCCTGACCGGCACCGGCGTGCTGACCGAGAGCAACAAGAAGTTCCTGCC
CTTCCAGCAGTTCGGCAGGGACATCGCCGACACCACCGACGCCGTGAGGG
ACCCTCAGACCCTGGAGATCCTGGACATCACCCCTTGCAGCTTCGGCGGC
GTGAGCGTGATCACCCCTGGCACCAACACCAGCAACCAGGTGGCCGTGCT
GTACCAGggcGTGAACTGCACCGAGGTGCCCGTGGCCATCCACGCCGACC
AGCTGACCCCTACCTGGAGGGTGTACTCCACCGGCAGCAACGTGTTCCAG
ACCAGGGCCGGCTGCCTGATCGGCGCCGAGCACGTGAACAACAGCTACGA
GTGCGACATCCCCATCGGCGCCGGCATCTGCGCCAGCTACCAGACCCAGA
CCAACAGCCCCgGGaGcGCCAGcAGCGTGGCCAGCCAGAGCATCATCGCC
TACACCATGAGCCTGGGCGtgGAGAACAGCGTGGCCTACAGCAACAACAG
CATCGCCATCCCCACCAACTTCACCATCAGCGTGACCACCGAGATCCTGC
CCGTGAGCATGACCAAGACCAGCGTGGACTGCACCATGTATATCTGCGGC
GACAGCACCGAGTGCAGCAACCTGCTGCTCCAGTACGGCAGCTTCTGCAC
CCAGCTGAACAGGGCCCTGACCGGCATCGCCGTGGAGCAGGACAAGAACA
CCCAGGAGGTGTTCGCCCAGGTGAAGCAGATCTACAAGACCCCTCCCATC
AAGGACTTCGGCGGCTTCAACTTCAGCCAGATCCTGCCCGACCCCAGCAA
GCCCAGCAAGAGGAGCTTCATCGAGGACCTGCTGTTAACAAGGTGACCCT
GGCCGACGCCGGCTTCATCAAGCAGTACGGCGACTGCCTGGGCGACATCG
CCGCCAGGGACCTGATCTGCGCCCAGAAGTTCAACGGCCTGACCGTGCTG
CCTCCCCTGCTGACCGACGAGATGATCGCCCAGTACACCAGCGCCCTGCT
GGCCGGCACCATCACCAGCGGCTGGACCTTCGGCGCCGGCGCCGCCCTGC
AGATCCCCTTCGCCATGCAGATGGCCTACAGGTTCAACGGCATCGGCGTG
ACCCAGAACGTGCTGTACGAGAACCAGAAGCTGATCGCCAACCAGTTCAA
CAGCGCCATCGGCAAGATCCAGGACAGCCTGAGCAGCACCGCCAGCGCCC
TGGGCAAGCTGCAGGACGTGGTGAACCAGAACGCCCAGGCCCTGAACACC
CTGGTGAAGCAGCTGAGCAGCAACTTCGGCGCCATCAGCAGCGTGCTGAA
CGACATCCTGAGCAGGCTGGACccacccGAGGCCGAGGTGCAGATCGACA
GGCTGATCACCGGCAGGCTGCAGAGCCTGCAGACCTACGTGACCCAGCAG
CTGATCAGGGCCGCCGAGATCAGGGCCAGCGCCAACCTGGCCGCCACCAA
GATGAGCGAGTGCGTGCTGGGCCAGAGCAAGAGGGTGGACTTCTGCGGCA
AGGGCTACCACCTGATGAGCTTCCCTCAGAGCGCCCCTCACGGCGTGGTG
TTCCTGCACGTGACCTACGTGCCCGCCCAGGAGAAGAACTTCACCACAGC
CCCTGCCATCTGCCACGACGGCAAGGCCCACTTCCCCAGGGAGGGCGTGT
TCGTGAGCAACGGCACCCACTGGTTCGTGACCCAGAGGAACTTCTACGAG
CCCCAGATCATCACCACCGACAACACCTTCGTGAGCGGCAACTGCGACGT
GGTGATCGGCATCGTGAACAACACCGTGTACGACCCTCTGCAGCCCGAGC
TGGACAGCTTCAAGGAGGAGCTGGACAAGTACTTCAAGAACCACACCAGC
CCCGACGTGGACCTGGGCGACATCAGCGGCATCAACGCCAGCGTGGTGAA
CATCCAGAAGGAGATCGACAGGCTGAACGAGGTGGCCAAGAACCTGAACG
AGAGCCTGATCGACCTGCAGGAGCTGGGCAAGTACGAGCAGTACATCAAG
TGGCCCTGGTACATCTGGCTGGGCTTCATCGCCGGCCTGATCGCCATCGT
GATGGTGACCATCATGCTGTGCTGCATGACCAGCTGCTGCAGCTGCCTGA
AGGGCTGCTGCAGCTGCGGCAGCTGCTGCAAGTTCGACGAGGACGACAGC
GAGCCCGTGCTGAAGGGCGTGAAGCTGCACTACACCTaAacTCGAGTATG
TTACGTGCAAAGGTGATTGTCACCCCCCGAAAGACCATATTGTGACACAC
CCTCAGTATCACGCCCAAACATTTACAGCCGCGGTGTCAAAAACCGCGTG
GACGTGGTTAACATCCCTGCTGGGAGGATCAGCCGTAATTATTATAATTG
GCTTGGTGCTGGCTACTATTGTGGCCATGTACGTGCTGACCAACCAGAAA
CATAATTGAATACAGCAGCAATTGGCAAGCTGCTTACATAGAACTCGCGG
CGATTGGCATGCCGCCTTAAAATTTTTATTTTATTTTTTCTTTTCTTTTC
CGAATCGGATTTTGTTTTTAATATTTCAAAAAAAAAAAAAAAAAAAAAAA
AATctagAAAAAAAAAAAAAAAAAAAAAAAAAAAAAAAAAAAAAAAAAAA
AAAAAAAAAAAAAAAAAAAAAAAAAAAAAAAAAAAAAaaaaaaaaaaaaa
aaaaaaa

SEQ ID NO:2 - mARM3280 (D614G)

atgggcggcgcatgagagaagcccagaccaattacctacccaaaatggag
aaagttcacgttgacatcgaggaagacagcccattcctcagagctttgca
gcggagcttcccgcagtttgaggtagaagccaagcaggtcactgataatg
accatgctaatgccagagcgttttcgcatctggcttcaaaactgatcgaa
acggaggtggacccatccgacacgatccttgacattggaagtgcgcccgc
ccgcagaatGTATTCTAAGCACAAGTATCATTGTATCtgtccgatgagat
gtgcggaagatccggacagattgtataagtatgcaactaagctgaagaaa
aactgtaaggaaataactgataaggaattggacaagaaaatgaaggagct
ggccgccgtcatgagcgaccctgacctggaaactgagactatgtgcctcc
acgacgacgagtcgtgtcgctacgaagggcaagtcgctgtttaccaggat
gtatacgcCGTcGACGGCCCCACCAGCCTGTACCACCAGGCCAACAAGGG
CGTGAGGGTGGCCTACTGGATCGGCTTCGACACCACACCCTTCATGTTCA
AGAACCTGGCCGGCGCCTACCCCAGCTACAGCACCAACTGGGCCGACGAG
ACAGTGCTGACCGCCAGGAACATCGGCCTGTGCAGCAGCGACGTGATGGA
GAGGAGCCGGAGGGGCATGAGCATCCTGAGGAAGAAGTACCTGAAGCCCA
GCAACAACGTGCTGTTCAGCGTGGGCAGCACCATCTACCACGAGAAGAGG
GACCTGCTGAGGAGCTGGCACCTGCCCAGCGTGTTCCACCTGAGGGGCAA
GCAGAACTACACCTGCAGGTGCGAGACAATCGTGAGCTGCGACGGCTACG
TGGTGAAGAGGATCGCCATCAGCCCCGGCCTGTACGGCAAGCCCAGCGGC
TACGCCGCCACCATGCACAGGGAGGGCTTCCTGTGCTGCAAGGTGACCGA
CACCCTGAACGGCGAGAGGGTGAGCTTCCCCGTGTGCACCTACGTGCCCG
CCACCCTGTGCGACCAGATGACCGGCATCCTGGCCACCGACGTGAGCGCC
GACGACGCCCAGAAGCTGCTGGTGGGCCTGAACCAGAGGATCGTGGTGAA
CGGCAGGACCCAGAGGAACACCAACACCATGAAGAACTACCTGCTGCCCG
TGGTGGCCCAGGCCTTCGCCAGGTGGGCCAAGGAGTACAAGGAGGACCAG
GAGGACGAGAGGCCCCTGGGCCTGAGGGACcGaCAGCTGGTGATGGGCTG
CTGCTGGGCCTTCAGGCGGCACAAGATCACCAGCATCTACAAGAGGCCCG
ACACCCAGACCATCATCAAGGTGAACAGCGACTTCCACAGCTTCGTGCTG
CCCAGGATCGGCAGCAACACCCTGGAGATCGGCCTGAGGACCCGGATCAG
GAAGATGCTGGAGGAGCACAAGGAGCCCAGCCCTCTGATCACCGCCGAGG
ACGTGCAGGAGGCCAAGTGCGCCGCCGACGAGGCCAAGGAGGTGAGGGAG
GCCGAGGAGCTGAGGGCCGCCCTGCCTCCCCTGGCCGCCGACGTGGAGGA
GCCCACCCTGGAGGCCGACGTGGACCTGATGCTGCAGGAGGCCGGCGCCG
GCAGCGTGGAGACACCCAGGGGCCTGATCAAGGTGACCAGCTACGACGGC
GAGGACAAGATCGGCAGCTACGCCGTGCTcAGCCCTCAGGCCGTGCTGAA
GTCCGAGAAGCTGAGCTGCATCCACCCTCTGGCCGAGCAGGTGATCGTGA
TCACCCACAGCGGCAGGAAGGGCAGGTACGCCGTGGAGCCCTACCACGGC
AAGGTGGTGGTCCCCGAGGGCCACGCCATCCCCGTGCAGGACTTCCAGGC
CCTGAGCGAGAGCGCCACCATCGTGTATAACGAGAGGGAGTTCGTGAACA
GGTACCTGCACCACATCGCCACCCACGGCGGCGCCCTGAACACCGACGAG
GAGTACTACAAGACCGTGAAGCCCAGCGAGCACGACGGCGAGTACCTGTA
CGACATCGACAGGAAGCAGTGCGTGAAGAAGGAGCTGGTGACCGGCCTGG
GCCTGACCGGCGAGCTGGTGGACCCTCCCTTCCACGAGTTCGCCTACGAG
AGCCTGAGGACCAGGCCCGCCGCTCCCTACCAGGTGCCCACCATCGGCGT
GTACGGCGTGCCCGGCAGCGGCAAGAGCGGCATCATCAAGAGCGCCGTGA
CCAAGAAGGACCTGGTGGTGAGCGCCAAGAAGGAGAACTGCGCCGAGATC
ATCAGGGACGTGAAGAAGATGAAGGGCCTGGACGTGAACGCCAGGACCGT
GGACAGCGTGCTcCTGAACGGCTGCAAGCACCCCGTGGAGACACTGTATA
TCGACGAGGCCTTCGCCTGCCACGCCGGCACCCTGAGGGCCCTGATCGCC
ATCATCAGGCCCAAGAAGGCCGTGCTGTGCGGCGACCCCAAGCAGTGCGG
CTTCTTCAACATGATGTGCCTGAAGGTGCACTTCAACCACGAGATCTGCA
CCCAGGTGTTCCACAAGAGCATCAGCAGGCGGTGCACCAAGAGCGTGACC
AGCGTGGTGAGCACCCTGTTCTACGACAAGAAGATGAGGACCACCAACCC
CAAGGAGACAAAGATCGTGATCGACACCACCGGCAGCACCAAGCCCAAGC
AGGACGACCTGATCCTGACCTGCTTCAGGGGCTGGGTGAAGCAGCTGCAG
ATCGACTACAAGGGCAACGAGATCATGACCGCCGCCGCTAGCCAGGGCCT
GACCAGGAAGGGCGTGTACGCCGTGAGGTACAAGGTGAACGAGAATCCCC
TGTACGCCCCTACCAGCGAGCACGTGAACGTcCTGCTGACCAGGACCGAG
GACAGGATCGTGTGGAAGACCCTGGCCGGCGACCCCTGGATCAAGACCCT
GACCGCCAAGTACCCCGGCAACTTCACCGCCACCATCGAGGAGTGGCAGG
CCGAGCACGACGCCATCATGAGGCACATCCTGGAGAGGCCCGACCCCACC
GACGTGTTCCAGAACAAGGCCAACGTGTGCTGGGCCAAGGCCCTGGTGCC
CGTGCTGAAGACCGCCGGCATCGACATGACCACCGAGCAGTGGAACACCG
TGGACTACTTCGAGACAGACAAGGCCCACAGCGCCGAGATCGTGCTGAAC
CAGCTGTGCGTGAGGTTCTTCGGCCTGGACCTGGACAGCGGCCTGTTCAG
CGCCCCTACCGTGCCCCTGAGCATCAGGAACAACCACTGGGACAACAGCC
CCAGCCCCAACATGTACGGCCTGAACAAGGAGGTGGTGAGGCAGCTGAGC
AGGCGGTACCCTCAGCTGCCCAGGGCCGTGGCCACCGGCAGGGTGTACGA
CATGAACACCGGCACCCTGAGGAACTACGACCCCAGGATCAACCTGGTGC
CCGTGAACAGGCGGCTGCCaCACGCCCTGGTGCTGCACCACAACGAGCAC
CCTCAGAGCGACTTCAGCAGCTTCGTGAGCAAGCTGAAGGGCAGGACCGT
GCTGGTGGTGGGCGAGAAGCTGAGCGTGCCCGGCAAGATGGTGGACTGGC
TGAGCGACAGGCCCGAGGCCACCTTCCGGGCCAGGCTGGACCTGGGCATC
CCCGGCGACGTGCCCAAGTACGACATCATCTTCGTGAACGTGAGGACCCC
TTACAAGTACCACCACTACCAGCAGTGCGAGGACCACGCCATCAAGCTGA
GCATGCTGACCAAGAAGGCCTGCCTGCACCTGAACCCCGGCGGCACCTGC
GTGAGCATCGGCTACGGCTACGCCGACAGGGCCAGCGAGAGCATCATCGG
CGCCATCGCCAGGCTGTTCAAGTTCAGCAGGGTGTGCAAGCCCAAGAGCA
GCCTGGAGGAGACAGAGGTGCTGTTCGTGTTCATCGGCTACGACCGGAAG
GCCAGGACCCACAACCCCTACAAGCTGAGCAGCACCCTGACCAACATCTA
CACCGGCAGCAGGCTGCACGAGGCCGGCTGCGCCCCTAGCTACCACGTGG
TGAGGGGCGACATCGCCACCGCCACCGAGGGCGTGATCATCAACGCCGCC
AACAGCAAGGGCCAGCCCGGCGGCGGGGTGTGCGGCGCCCTGTATAAGAA
GTTCCCCGAGAGCTTCGACCTGCAGCCCATCGAGGTGGGCAAGGCCAGGC
TGGTGAAGGGCGCCGCCAAGCACATCATCCACGCCGTGGGCCCCAACTTC
AACAAGGTGAGCGAGGTGGAGGGCGACAAGCAGCTGGCCGAGGCCTACGA
GAGCATCGCCAAGATCGTGAACGACAACAACTACAAGAGCGTGGCCATCC
CTCTGCTGAGCACCGGCATCTTCAGCGGCAACAAGGACAGGCTGACCCAG
AGCCTGAACCACCTGCTGACCGCCCTGGACACCACCGACGCCGACGTGGC
CATCTACTGCAGGGACAAGAAGTGGGAGATGACCCTGAAGGAGGCCGTGG
CCAGGCGGGAGGCCGTGGAGGAGATCTGCATCAGCGACGACAGCAGCGTG
ACgGAGCCCGACGCCGAGCTGGTGAGGGTGCACCCCAAGAGCAGCCTGGC
CGGCAGGAAGGGCTACAGCACCAGCGACGGCAAGACCTTCAGCTACCTGG
AGGGCACCAAGTTCCACCAGGCCGCCAAGGACATCGCCGAGATCAACGCC
ATGTGGCCCGTGGCCACCGAGGCCAACGAGCAGGTGTGCATGTATATCCT
GGGCGAGAGCATGAGCAGCATCAGGAGCAAGTGCCCCGTGGAGGAGAGCG
AGGCCAGCACCCCTCCCAGCACCCTGCCCTGCCTGTGCATCCACGCCATG
ACCCCTGAGAGGGTGCAGCGGCTGAAGGCCAGCAGGCCCGAGCAGATCAC
CGTGTGCAGCAGCTTCCCTCTGCCCAAGTACcGGATCACCGGCGTGCAGA
AGATCCAGTGCAGCCAGCCCATCCTGTTCAGCCCCAAGGTGCCCGCCTAC
ATCCACCCCAGGAAGTACCTGGTGGAGACACCCCCCGTGGACGAGACACC
CGAGCCCAGCGCCGAGAACCAGAGCACCGAGGGCACCCCTGAGCAGCCTC
CCCTGATCACCGAGGACGAGACAAGGACCAGGACgCCcGAGCCCATCATC
ATTGAGGAGGAAGAGGAGGACAGCATCAGCCTGCTGAGCGACGGCCCCAC
CCACCAGGTGCTGCAGGTGGAGGCCGACATCCACGGCCCTCCCAGCGTGA
GCAGCTCCAGCTGGAGCATCCCTCACGCCAGCGACTTCGACGTGGACAGC
CTGAGCATCCTGGACACCCTGGAGGGCGCCAGCGTGACCAGCGGCGCCAC
CAGCGCCGAGACAAACAGCTACTTCGCCAAGAGCATGGAGTTCCTGGCCA
GGCCCGTGCCCGCCCCTAGGACCGTGTTCAGGAACCCTCCCCACCCCGCC
CCTAGGACCAGGACCCCTAGCCTGGCCCCAGCAGGGCCTGCAGCAGGACC
AGCCTGGTGAGCACCCCTCCCGGCGTGAACcGGGTGATCACCAGGGAGGA
GCTGGAGGCCCTGACCCCTAGCAGGACCCCTAGCAGGAGCGTGAGCAGGA
CCAGCCTGGTGAGCAACCCTCCCGGCGTGAACcGGGTGATCACCAGGGAG
GAGTTCGAGGCCTTCGTGGCCCAGCAGCAAAGGCGGTTCGACGCCGGCGC
CTACATCTTCAGCAGCGACACCGGCCAGGGCCACCTGCAGCAGAAGTCCG
TGAGGCAGACCGTGCTGAGCGAGGTGGTcCTGGAGAGGACgGAGCTGGAG
ATCAGCTACGCCCCTAGGCTGGACCAGGAGAAGGAGGAGCTGCTGAGGAA
GAAGCTGCAGCTGAACCCCACCCCTGCCAACAGGAGCAGGTACCAGAGCA
GGAAGGTGGAGAACATGAAGGCCATCACCGCCAGGCGGATCCTGCAGGGC
CTGGGCCACTACCTGAAGGCCGAGGGCAAGGTGGAGTGCTACAGGACCCT
GCACCCCGTGCCCCTGTACTCCAGCTCCGTGAACAGGGCCTTCAGCAGCC
CCAAGGTGGCCGTGGAGGCCTGCAACGCCATGCTGAAGGAGAACTTCCCC
ACCGTGGCCAGCTACTGCATCATCCCCGAGTACGACGCCTACCTGGACAT
GGTGGACGGCGCCAGCTGCTGCCTGGACACCGCCAGCTTCTGCCCCGCCA
AGCTGAGGAGCTTCCCCAAGAAGCACAGCTACCTGGAGCCCACCATCAGG
AGCGCCGTGCCCAGCGCCATCCAGAACACCCTGCAGAACGTGCTGGCCGC
CGCTACCAAGAGGAACTGCAACGTGACCCAGATGAGGGAGCTGCCCGTGC
TGGACAGCGCCGCCTTCAACGTGGAGTGCTTCAAGAAGTACGCCTGCAAC
AACGAGTACTGGGAGACATTCAAGGAGAACCCCATCAGGCTGACCGAGGA
GAACGTGGTGAACTACATCACCAAGCTGAAGGGCCCCAAGGCCGCCGCTC
TGTTCGCCAAGACCCACAACCTGAACATGCTcCAGGACATCCCTATGGAC
AGGTTCGTGATGGACCTGAAGAGGGACGTGAAGGTGACCCCTGGCACCAA
GCACACCGAGGAGAGGCCCAAGGTGCAGGTGATCCAGGCCGCCGACCCTC
TGGCCACCGCCTACCTGTGCGGCATCCACAGGGAGCTGGTGAGGCGGCTG
AACGCCGTcCTGCTGCCCAACATCCACACCCTGTTCGACATGAGCGCCGA
GGACTTCGACGCCATCATCGCCGAGCACTTCCAGCCCGGCGACTGCGTGC
TGGAGACAGACATCGCCAGCTTCGACAAGAGCGAGGACGACGCTATGGCC
CTGACCGCCCTGATGATCCTGGAGGACCTGGGCGTGGACGCCGAGCTGCT
GACCCTGATCGAGGCCGCCTTCGGCGAGATCAGCAGCATCCACCTGCCCA
CCAAGACCAAGTTCAAGTTCGGCGCCATGATGAAGTCCGGCATGTTCCTG
ACCCTGTTCGTGAACACCGTGATCAACATCGTGATCGCCAGCAGGGTGCT
GCGGGAGAGGCTGACCGGCAGCCCCTGCGCCGCCTTCATCGGCGACGACA
ACATCGTGAAGGGCGTGAAGTCCGACAAGCTGATGGCCGACAGGTGCGCC
ACCTGGCTGAACATGGAGGTGAAGATCATCGACGCCGTGGTGGGCGAGAA
GGCCCCTTACTTCTGCGGCGGCTTCATCCTGTGCGACAGCGTGACCGGCA
CCGCCTGCAGGGTGGCCGACCCTCTGAAGAGGCTGTTCAAGCTGGGCAAG
CCCCTGGCCGCCGACGACGAGCACGACGACGATAGGCGGAGGGCCCTGCA
CGAGGAGAGCACCAGGTGGAACcGGGTGGGCATCCTGAGCGAGCTGTGCA
AGGCCGTGGAGAGCAGGTACGAGACAGTGGGCACCAGCATCATCGTGATG
GCCATGACCACCCTGGCCAGCAGCGTcAAGTCCTTCAGCTACCTGAGGGG
GGCCCCTATAACTCTCTACGGCTAACCTGAATGGACTACGACATAGTCTA
GTCCGCCAAGGCCGCCACCATGTTCGTGTTCCTGGTGCTGCTGCCCCTGG
TGTCTAGCCAGTGCGTGAACCTGACCACCAGGACCCAGCTGCCTCCCGCC
TACACCAACAGCTTCACCAGGGGCGTGTACTACCCCGACAAGGTGTTCAG
GAGCAGCGTGCTGCACAGCACCCAGGACCTGTTCCTGCCCTTCTTCAGCA
ACGTGACCTGGTTCCACGCCATCCACGTGAGCGGCACCAACGGCACCAAG
AGGTTCGACAACCCCGTGCTGCCCTTCAACGACGGCGTGTACTTCGCCAG
CACCGAGAAGTCCAACATCATCAGGGGCTGGATCTTCGGCACCACCCTGG
ACAGCAAGACCCAGAGCCTGCTGATCGTGAACAACGCCACCAACGTGGTG
ATCAAGGTGTGCGAGTTCCAGTTCTGCAACGACCCCTTCCTGGGCGTGTA
CTACCACAAGAACAACAAGAGCTGGATGGAGAGCGAGTTCAGGGTGTACT
CCAGCGCCAACAACTGCACCTTCGAGTACGTGAGCCAGCCCTTCCTGATG
GACCTGGAGGGCAAGCAGGGCAACTTCAAGAACCTGAGGGAGTTCGTGTT
CAAGAACATCGACGGCTACTTCAAGATCTACAGCAAGCACACCCCTATCA
ACCTGGTGAGGGACCTGCCCCAGGGCTTCAGCGCCCTGGAGCCCCTGGTG
GACCTGCCCATCGGCATCAACATCACCAGGTTCCAGACCCTGCTGGCCCT
GCACAGGAGCTACCTGACCCCTGGCGACAGCAGCTCCGGCTGGACCGCCG
GCGCCGCCGCTTACTACGTGGGCTACCTGCAGCCCAGGACCTTCCTGCTG
AAGTACAACGAGAACGGCACCATCACCGACGCCGTGGACTGCGCCCTGGA
CCCTCTGAGCGAGACAAAGTGCACCCTGAAGTCCTTCACCGTGGAGAAGG
GCATCTACCAGACCAGCAACTTCAGGGTGCAGCCCACCGAGAGCATCGTG
AGGTTCCCCAACATCACCAACCTGTGCCCCTTCGGCGAGGTGTTCAACGC
CACCAGGTTCGCCAGCGTGTACGCCTGGAACAGGAAGAGGATCAGCAACT
GCGTGGCCGACTACAGCGTGCTGTATAACAGCGCCAGCTTCAGCACCTTC
AAGTGCTACGGCGTGAGCCCCACCAAGCTGAACGACCTGTGCTTCACCAA
CGTGTACGCCGACAGCTTCGTGATCAGGGGCGACGAGGTGAGGCAGATCG
CCCCTGGCCAGACCGGCAAGATCGCCGACTACAACTACAAGCTGCCCGAC
GACTTCACCGGCTGCGTGATCGCCTGGAACAGCAACAACCTGGACAGCAA
GGTGGGCGGCAACTACAACTACCTGTACCGGCTGTTCAGAAAGAGCAACC
TGAAGCCCTTCGAGAGGGACATCAGCACCGAGATCTACCAGGCCGGCAGC
ACCCCTTGCAACGGCGTGGAGGGCTTCAACTGCTACTTCCCTCTGCAGAG
CTACGGCTTCCAGCCCACCAACGGCGTGGGCTACCAGCCCTACAGGGTGG
TGGTCCTGAGCTTCGAGCTGCTGCACGCCCCTGCCACCGTGTGCGGCCCC
AAGAAGTCCACCAACCTGGTGAAGAACAAGTGCGTGAACTTCAACTTCAA
CGGCCTGACCGGCACCGGCGTGCTGACCGAGAGCAACAAGAAGTTCCTGC
CCTTCCAGCAGTTCGGCAGGGACATCGCCGACACCACCGACGCCGTGAGG
GACCCTCAGACCCTGGAGATCCTGGACATCACCCCTTGCAGCTTCGGCGG
CGTGAGCGTGATCACCCCTGGCACCAACACCAGCAACCAGGTGGCCGTGC
TGTACCAGggcGTGAACTGCACCGAGGTGCCCGTGGCCATCCACGCCGAC
CAGCTGACCCCTACCTGGAGGGTGTACTCCACCGGCAGCAACGTGTTCCA
GACCAGGGCCGGCTGCCTGATCGGCGCCGAGCACGTGAACAACAGCTACG
AGTGCGACATCCCCATCGGCGCCGGCATCTGCGCCAGCTACCAGACCCAG
ACCAACAGCCCCgGGaGcGCCAGcAGCGTGGCCAGCCAGAGCATCATCGC
CTACACCATGAGCCTGGGCGCCGAGAACAGCGTGGCCTACAGCAACAACA
GCATCGCCATCCCCACCAACTTCACCATCAGCGTGACCACCGAGATCCTG
CCCGTGAGCATGACCAAGACCAGCGTGGACTGCACCATGTATATCTGCGG
CGACAGCACCGAGTGCAGCAACCTGCTGCTCCAGTACGGCAGCTTCTGCA
CCCAGCTGAACAGGGCCCTGACCGGCATCGCCGTGGAGCAGGACAAGAAC
ACCCAGGAGGTGTTCGCCCAGGTGAAGCAGATCTACAAGACCCCTCCCAT
CAAGGACTTCGGCGGCTTCAACTTCAGCCAGATCCTGCCCGACCCCAGCA
AGCCCAGCAAGAGGAGCTTCATCGAGGACCTGCTGTTCAACAAGGTGACC
CTGGCCGACGCCGGCTTCATCAAGCAGTACGGCGACTGCCTGGGCGACAT
CGCCGCCAGGGACCTGATCTGCGCCCAGAAGTTCAACGGCCTGACCGTGC
TGCCTCCCCTGCTGACCGACGAGATGATCGCCCAGTACACCAGCGCCCTG
CTGGCCGGCACCATCACCAGCGGCTGGACCTTCGGCGCCGGCGCCGCCCT
GCAGATCCCCTTCGCCATGCAGATGGCCTACAGGTTCAACGGCATCGGCG
TGACCCAGAACGTGCTGTACGAGAACCAGAAGCTGATCGCCAACCAGTTC
AACAGCGCCATCGGCAAGATCCAGGACAGCCTGAGCAGCACCGCCAGCGC
CCTGGGCAAGCTGCAGGACGTGGTGAACCAGAACGCCCAGGCCCTGAACA
CCCTGGTGAAGCAGCTGAGCAGCAACTTCGGCGCCATCAGCAGCGTGCTG
AACGACATCCTGAGCAGGCTGGACccacccGAGGCCGAGGTGCAGATCGA
CAGGCTGATCACCGGCAGGCTGCAGAGCCTGCAGACCTACGTGACCCAGC
AGCTGATCAGGGCCGCCGAGATCAGGGCCAGCGCCAACCTGGCCGCCACC
AAGATGAGCGAGTGCGTGCTGGGCCAGAGCAAGAGGGTGGACTTCTGCGG
CAAGGGCTACCACCTGATGAGCTTCCCTCAGAGCGCCCCTCACGGCGTGG
TGTTCCTGCACGTGACCTACGTGCCCGCCCAGGAGAAGAACTTCACCACA
GCCCCTGCCATCTGCCACGACGGCAAGGCCCACTTCCCCAGGGAGGGCGT
GTTCGTGAGCAACGGCACCCACTGGTTCGTGACCCAGAGGAACTTCTACG
AGCCCCAGATCATCACCACCGACAACACCTTCGTGAGCGGCAACTGCGAC
GTGGTGATCGGCATCGTGAACAACACCGTGTACGACCCTCTGCAGCCCGA
GCTGGACAGCTTCAAGGAGGAGCTGGACAAGTACTTCAAGAACCACACCA
GCCCCGACGTGGACCTGGGCGACATCAGCGGCATCAACGCCAGCGTGGTG
AACATCCAGAAGGAGATCGACAGGCTGAACGAGGTGGCCAAGAACCTGAA
CGAGAGCCTGATCGACCTGCAGGAGCTGGGCAAGTACGAGCAGTACATCA
AGTGGCCCTGGTACATCTGGCTGGGCTTCATCGCCGGCCTGATCGCCATC
GTGATGGTGACCATCATGCTGTGCTGCATGACCAGCTGCTGCAGCTGCCT
GAAGGGCTGCTGCAGCTGCGGCAGCTGCTGCAAGTTCGACGAGGACGACA
GCGAGCCCGTGCTGAAGGGCGTGAAGCTGCACTACACCTaAaCTCGAGTA
TGTTACGTGCAAAGGTGATTGTCACCCCCCGAAAGACCATATTGTGACAC
ACCCTCAGTATCACGCCCAAACATTTACAGCCGCGGTGTCAAAAACCGCG
TGGACGTGGTTAACATCCCTGCTGGGAGGATCAGCCGTAATTATTATAAT
TGGCTTGGTGCTGGCTACTATTGTGGCCATGTACGTGCTGACCAACCAGA
AACATAATTGAATACAGCAGCAATTGGCAAGCTGCTTACATAGAACTCGC
GGCGATTGGCATGCCGCCTTAAAATTTTTATTTTATTTTTTCTTTTCTTT
TCCGAATCGGATTTTGTTTTTAATATTTCAAAAAAAAAAAAAAAAAAAAA
AAAATctagAAAAAAAAAAAAAAAAAAAAAAAAAAAAAAAAAAAAAAAAA
AAAAAAAAAAAAAAAAAAAAAAAAAAAAAAAAAAAAAAAaaaaaaaaaaa
aaaaaaaaa

SEQ ID NO:3 - mARM3333 (UK B.1.1.7)

atgggcggcgcatgagagaagcccagaccaattacctacccaaaatggag
aaagttcacgttgacatcgaggaagacagcccattcctcagagctttgca
gcggagcttcccgcagtttgaggtagaagccaagcaggtcactgataatg
accatgctaatgccagagcgttttcgcatctggcttcaaaactgatcgaa
acggaggtggacccatccgacacgatccttgacattggaagtgcgcccgc
ccgcagaatGTATTCTAAGCACAAGTATCATTGTATCtgtccgatgagat
gtgcggaagatccggacagattgtataagtatgcaactaagctgaagaaa
aactgtaaggaaataactgataaggaattggacaagaaaatgaaggagct
ggccgccgtcatgagcgaccctgacctggaaactgagactatgtgcctcc
acgacgacgagtcgtgtcgctacgaagggcaagtcgctgtttaccaggat
gtatacgcCGTcGACGGCCCCACCAGCCTGTACCACCAGGCCAACAAGGG
CGTGAGGGTGGCCTACTGGATCGGCTTCGACACCACACCCTTCATGTTCA
AGAACCTGGCCGGCGCCTACCCCAGCTACAGCACCAACTGGGCCGACGAG
ACAGTGCTGACCGCCAGGAACATCGGCCTGTGCAGCAGCGACGTGATGGA
GAGGAGCCGGAGGGGCATGAGCATCCTGAGGAAGAAGTACCTGAAGCCCA
GCAACAACGTGCTGTTCAGCGTGGGCAGCACCATCTACCACGAGAAGAGG
GACCTGCTGAGGAGCTGGCACCTGCCCAGCGTGTTCCACCTGAGGGGCAA
GCAGAACTACACCTGCAGGTGCGAGACAATCGTGAGCTGCGACGGCTACG
TGGTGAAGAGGATCGCCATCAGCCCCGGCCTGTACGGCAAGCCCAGCGGC
TACGCCGCCACCATGCACAGGGAGGGCTTCCTGTGCTGCAAGGTGACCGA
CACCCTGAACGGCGAGAGGGTGAGCTTCCCCGTGTGCACCTACGTGCCCG
CCACCCTGTGCGACCAGATGACCGGCATCCTGGCCACCGACGTGAGCGCC
GACGACGCCCAGAAGCTGCTGGTGGGCCTGAACCAGAGGATCGTGGTGAA
CGGCAGGACCCAGAGGAACACCAACACCATGAAGAACTACCTGCTGCCCG
TGGTGGCCCAGGCCTTCGCCAGGTGGGCCAAGGAGTACAAGGAGGACCAG
GAGGACGAGAGGCCCCTGGGCCTGAGGGACcGaCAGCTGGTGATGGGCTG
CTGCTGGGCCTTCAGGCGGCACAAGATCACCAGCATCTACAAGAGGCCCG
ACACCCAGACCATCATCAAGGTGAACAGCGACTTCCACAGCTTCGTGCTG
CCCAGGATCGGCAGCAACACCCTGGAGATCGGCCTGAGGACCCGGATCAG
GAAGATGCTGGAGGAGCACAAGGAGCCCAGCCCTCTGATCACCGCCGAGG
ACGTGCAGGAGGCCAAGTGCGCCGCCGACGAGGCCAAGGAGGTGAGGGAG
GCCGAGGAGCTGAGGGCCGCCCTGCCTCCCCTGGCCGCCGACGTGGAGGA
GCCCACCCTGGAGGCCGACGTGGACCTGATGCTGCAGGAGGCCGGCGCCG
GCAGCGTGGAGACACCCAGGGGCCTGATCAAGGTGACCAGCTACGACGGC
GAGGACAAGATCGGCAGCTACGCCGTGCTcAGCCCTCAGGCCGTGCTGAA
GTCCGAGAAGCTGAGCTGCATCCACCCTCTGGCCGAGCAGGTGATCGTGA
TCACCCACAGCGGCAGGAAGGGCAGGTACGCCGTGGAGCCCTACCACGGC
AAGGTGGTGGTCCCCGAGGGCCACGCCATCCCCGTGCAGGACTTCCAGGC
CCTGAGCGAGAGCGCCACCATCGTGTATAACGAGAGGGAGTTCGTGAACA
GGTACCTGCACCACATCGCCACCCACGGCGGCGCCCTGAACACCGACGAG
GAGTACTACAAGACCGTGAAGCCCAGCGAGCACGACGGCGAGTACCTGTA
CGACATCGACAGGAAGCAGTGCGTGAAGAAGGAGCTGGTGACCGGCCTGG
GCCTGACCGGCGAGCTGGTGGACCCTCCCTTCCACGAGTTCGCCTACGAG
AGCCTGAGGACCAGGCCCGCCGCTCCCTACCAGGTGCCCACCATCGGCGT
GTACGGCGTGCCCGGCAGCGGCAAGAGCGGCATCATCAAGAGCGCCGTGA
CCAAGAAGGACCTGGTGGTGAGCGCCAAGAAGGAGAACTGCGCCGAGATC
ATCAGGGACGTGAAGAAGATGAAGGGCCTGGACGTGAACGCCAGGACCGT
GGACAGCGTGCTcCTGAACGGCTGCAAGCACCCCGTGGAGACACTGTATA
TCGACGAGGCCTTCGCCTGCCACGCCGGCACCCTGAGGGCCCTGATCGCC
ATCATCAGGCCCAAGAAGGCCGTGCTGTGCGGCGACCCCAAGCAGTGCGG
CTTCTTCAACATGATGTGCCTGAAGGTGCACTTCAACCACGAGATCTGCA
CCCAGGTGTTCCACAAGAGCATCAGCAGGCGGTGCACCAAGAGCGTGACC
AGCGTGGTGAGCACCCTGTTCTACGACAAGAAGATGAGGACCACCAACCC
CAAGGAGACAAAGATCGTGATCGACACCACCGGCAGCACCAAGCCCAAGC
AGGACGACCTGATCCTGACCTGCTTCAGGGGCTGGGTGAAGCAGCTGCAG
ATCGACTACAAGGGCAACGAGATCATGACCGCCGCCGCTAGCCAGGGCCT
GACCAGGAAGGGCGTGTACGCCGTGAGGTACAAGGTGAACGAGAATCCCC
TGTACGCCCCTACCAGCGAGCACGTGAACGTcCTGCTGACCAGGACCGAG
GACAGGATCGTGTGGAAGACCCTGGCCGGCGACCCCTGGATCAAGACCCT
GACCGCCAAGTACCCCGGCAACTTCACCGCCACCATCGAGGAGTGGCAGG
CCGAGCACGACGCCATCATGAGGCACATCCTGGAGAGGCCCGACCCCACC
GACGTGTTCCAGAACAAGGCCAACGTGTGCTGGGCCAAGGCCCTGGTGCC
CGTGCTGAAGACCGCCGGCATCGACATGACCACCGAGCAGTGGAACACCG
TGGACTACTTCGAGACAGACAAGGCCCACAGCGCCGAGATCGTGCTGAAC
CAGCTGTGCGTGAGGTTCTTCGGCCTGGACCTGGACAGCGGCCTGTTCAG
CGCCCCTACCGTGCCCCTGAGCATCAGGAACAACCACTGGGACAACAGCC
CCAGCCCCAACATGTACGGCCTGAACAAGGAGGTGGTGAGGCAGCTGAGC
AGGCGGTACCCTCAGCTGCCCAGGGCCGTGGCCACCGGCAGGGTGTACGA
CATGAACACCGGCACCCTGAGGAACTACGACCCCAGGATCAACCTGGTGC
CCGTGAACAGGCGGCTGCCaCACGCCCTGGTGCTGCACCACAACGAGCAC
CCTCAGAGCGACTTCAGCAGCTTCGTGAGCAAGCTGAAGGGCAGGACCGT
GCTGGTGGTGGGCGAGAAGCTGAGCGTGCCCGGCAAGATGGTGGACTGGC
TGAGCGACAGGCCCGAGGCCACCTTCCGGGCCAGGCTGGACCTGGGCATC
CCCGGCGACGTGCCCAAGTACGACATCATCTTCGTGAACGTGAGGACCCC
TTACAAGTACCACCACTACCAGCAGTGCGAGGACCACGCCATCAAGCTGA
GCATGCTGACCAAGAAGGCCTGCCTGCACCTGAACCCCGGCGGCACCTGC
GTGAGCATCGGCTACGGCTACGCCGACAGGGCCAGCGAGAGCATCATCGG
CGCCATCGCCAGGCTGTTCAAGTTCAGCAGGGTGTGCAAGCCCAAGAGCA
GCCTGGAGGAGACAGAGGTGCTGTTCGTGTTCATCGGCTACGACCGGAAG
GCCAGGACCCACAACCCCTACAAGCTGAGCAGCACCCTGACCAACATCTA
CACCGGCAGCAGGCTGCACGAGGCCGGCTGCGCCCCTAGCTACCACGTGG
TGAGGGGCGACATCGCCACCGCCACCGAGGGCGTGATCATCAACGCCGCC
AACAGCAAGGGCCAGCCCGGCGGCGGGGTGTGCGGCGCCCTGTATAAGAA
GTTCCCCGAGAGCTTCGACCTGCAGCCCATCGAGGTGGGCAAGGCCAGGC
TGGTGAAGGGCGCCGCCAAGCACATCATCCACGCCGTGGGCCCCAACTTC
AACAAGGTGAGCGAGGTGGAGGGCGACAAGCAGCTGGCCGAGGCCTACGA
GAGCATCGCCAAGATCGTGAACGACAACAACTACAAGAGCGTGGCCATCC
CTCTGCTGAGCACCGGCATCTTCAGCGGCAACAAGGACAGGCTGACCCAG
AGCCTGAACCACCTGCTGACCGCCCTGGACACCACCGACGCCGACGTGGC
CATCTACTGCAGGGACAAGAAGTGGGAGATGACCCTGAAGGAGGCCGTGG
CCAGGCGGGAGGCCGTGGAGGAGATCTGCATCAGCGACGACAGCAGCGTG
ACgGAGCCCGACGCCGAGCTGGTGAGGGTGCACCCCAAGAGCAGCCTGGC
CGGCAGGAAGGGCTACAGCACCAGCGACGGCAAGACCTTCAGCTACCTGG
AGGGCACCAAGTTCCACCAGGCCGCCAAGGACATCGCCGAGATCAACGCC
ATGTGGCCCGTGGCCACCGAGGCCAACGAGCAGGTGTGCATGTATATCCT
GGGCGAGAGCATGAGCAGCATCAGGAGCAAGTGCCCCGTGGAGGAGAGCG
AGGCCAGCACCCCTCCCAGCACCCTGCCCTGCCTGTGCATCCACGCCATG
ACCCCTGAGAGGGTGCAGCGGCTGAAGGCCAGCAGGCCCGAGCAGATCAC
CGTGTGCAGCAGCTTCCCTCTGCCCAAGTACcGGATCACCGGCGTGCAGA
AGATCCAGTGCAGCCAGCCCATCCTGTTCAGCCCCAAGGTGCCCGCCTAC
ATCCACCCCAGGAAGTACCTGGTGGAGACACCCCCCGTGGACGAGACACC
CGAGCCCAGCGCCGAGAACCAGAGCACCGAGGGCACCCCTGAGCAGCCTC
CCCTGATCACCGAGGACGAGACAAGGACCAGGACgCCcGAGCCCATCATC
ATTGAGGAGGAAGAGGAGGACAGCATCAGCCTGCTGAGCGACGGCCCCAC
CCACCAGGTGCTGCAGGTGGAGGCCGACATCCACGGCCCTCCCAGCGTGA
GCAGCTCCAGCTGGAGCATCCCTCACGCCAGCGACTTCGACGTGGACAGC
CTGAGCATCCTGGACACCCTGGAGGGCGCCAGCGTGACCAGCGGCGCCAC
CAGCGCCGAGACAAACAGCTACTTCGCCAAGAGCATGGAGTTCCTGGCCA
GGCCCGTGCCCGCCCCTAGGACCGTGTTCAGGAACCCTCCCCACCCCGCC
CCTAGGACCAGGACCCCTAGCCTGGCCCCTAGCAGGGCCTGCAGCAGGAC
CAGCCTGGTGAGCACCCCTCCCGGCGTGAACcGGGTGATCACCAGGGAGG
AGCTGGAGGCCCTGACCCCTAGCAGGACCCCTAGCAGGAGCGTGAGCAGG
ACCAGCCTGGTGAGCAACCCTCCCGGCGTGAACcGGGTGATCACCAGGGA
GGAGTTCGAGGCCTTCGTGGCCCAGCAGCAAAGGCGGTTCGACGCCGGCG
CCTACATCTTCAGCAGCGACACCGGCCAGGGCCACCTGCAGCAGAAGTCC
GTGAGGCAGACCGTGCTGAGCGAGGTGGTcCTGGAGAGGACgGAGCTGGA
GATCAGCTACGCCCCTAGGCTGGACCAGGAGAAGGAGGAGCTGCTGAGGA
AGAAGCTGCAGCTGAACCCCACCCCTGCCAACAGGAGCAGGTACCAGAGC
AGGAAGGTGGAGAACATGAAGGCCATCACCGCCAGGCGGATCCTGCAGGG
CCTGGGCCACTACCTGAAGGCCGAGGGCAAGGTGGAGTGCTACAGGACCC
TGCACCCCGTGCCCCTGTACTCCAGCTCCGTGAACAGGGCCTTCAGCAGC
CCCAAGGTGGCCGTGGAGGCCTGCAACGCCATGCTGAAGGAGAACTTCCC
CACCGTGGCCAGCTACTGCATCATCCCCGAGTACGACGCCTACCTGGACA
TGGTGGACGGCGCCAGCTGCTGCCTGGACACCGCCAGCTTCTGCCCCGCC
AAGCTGAGGAGCTTCCCCAAGAAGCACAGCTACCTGGAGCCCACCATCAG
GAGCGCCGTGCCCAGCGCCATCCAGAACACCCTGCAGAACGTGCTGGCCG
CCGCTACCAAGAGGAACTGCAACGTGACCCAGATGAGGGAGCTGCCCGTG
CTGGACAGCGCCGCCTTCAACGTGGAGTGCTTCAAGAAGTACGCCTGCAA
CAACGAGTACTGGGAGACATTCAAGGAGAACCCCATCAGGCTGACCGAGG
AGAACGTGGTGAACTACATCACCAAGCTGAAGGGCCCCAAGGCCGCCGCT
CTGTTCGCCAAGACCCACAACCTGAACATGCTcCAGGACATCCCTATGGA
CAGGTTCGTGATGGACCTGAAGAGGGACGTGAAGGTGACCCCTGGCACCA
AGCACACCGAGGAGAGGCCCAAGGTGCAGGTGATCCAGGCCGCCGACCCT
CTGGCCACCGCCTACCTGTGCGGCATCCACAGGGAGCTGGTGAGGCGGCT
GAACGCCGTcCTGCTGCCCAACATCCACACCCTGTTCGACATGAGCGCCG
AGGACTTCGACGCCATCATCGCCGAGCACTTCCAGCCCGGCGACTGCGTG
CTGGAGACAGACATCGCCAGCTTCGACAAGAGCGAGGACGACGCTATGGC
CCTGACCGCCCTGATGATCCTGGAGGACCTGGGCGTGGACGCCGAGCTGC
TGACCCTGATCGAGGCCGCCTTCGGCGAGATCAGCAGCATCCACCTGCCC
ACCAAGACCAAGTTCAAGTTCGGCGCCATGATGAAGTCCGGCATGTTCCT
GACCCTGTTCGTGAACACCGTGATCAACATCGTGATCGCCAGCAGGGTGC
TGCGGGAGAGGCTGACCGGCAGCCCCTGCGCCGCCTTCATCGGCGACGAC
AACATCGTGAAGGGCGTGAAGTCCGACAAGCTGATGGCCGACAGGTGCGC
CACCTGGCTGAACATGGAGGTGAAGATCATCGACGCCGTGGTGGGCGAGA
AGGCCCCTTACTTCTGCGGCGGCTTCATCCTGTGCGACAGCGTGACCGGC
ACCGCCTGCAGGGTGGCCGACCCTCTGAAGAGGCTGTTCAAGCTGGGCAA
GCCCCTGGCCGCCGACGACGAGCACGACGACGATAGGCGGAGGGCCCTGC
ACGAGGAGAGCACCAGGTGGAACcGGGTGGGCATCCTGAGCGAGCTGTGC
AAGGCCGTGGAGAGCAGGTACGAGACAGTGGGCACCAGCATCATCGTGAT
GGCCATGACCACCCTGGCCAGCAGCGTcAAGTCCTTCAGCTACCTGAGGG
GGGCCCCTATAACTCTCTACGGCTAACCTGAATGGACTACGACATAGTCT
AGTCCGCCAAGGCCGCCACcATGTTCGTGTTCCTGGTGCTGCTGCCCCTG
GTGTCTAGCCAGTGCGTGAACCTGACCACCAGGACCCAGCTGCCTCCCGC
CTACACCAACAGCTTCACCAGGGGCGTGTACTACCCCGACAAGGTGTTCA
GGAGCAGCGTGCTGCACAGCACCCAGGACCTGTTCCTGCCCTTCTTCAGC
AACGTGACCTGGTTCCACGCCATCAGCGGCACCAACGGCACCAAGAGGTT
CGACAACCCCGTGCTGCCCTTCAACGACGGCGTGTACTTCGCCAGCACCG
AGAAGTCCAACATCATCAGGGGCTGGATCTTCGGCACCACCCTGGACAGC
AAGACCCAGAGCCTGCTGATCGTGAACAACGCCACCAACGTGGTGATCAA
GGTGTGCGAGTTCCAGTTCTGCAACGACCCCTTCCTGGGCGTGTACCACA
AGAACAACAAGAGCTGGATGGAGAGCGAGTTCAGGGTGTACTCCAGCGCC
AACAACTGCACCTTCGAGTACGTGAGCCAGCCCTTCCTGATGGACCTGGA
GGGCAAGCAGGGCAACTTCAAGAACCTGAGGGAGTTCGTGTTCAAGAACA
TCGACGGCTACTTCAAGATCTACAGCAAGCACACCCCTATCAACCTGGTG
AGGGACCTGCCCCAGGGCTTCAGCGCCCTGGAGCCCCTGGTGGACCTGCC
CATCGGCATCAACATCACCAGGTTCCAGACCCTGCTGGCCCTGCACAGGA
GCTACCTGACCCCTGGCGACAGCAGCTCCGGCTGGACCGCCGGCGCCGCC
GCTTACTACGTGGGCTACCTGCAGCCCAGGACCTTCCTGCTGAAGTACAA
CGAGAACGGCACCATCACCGACGCCGTGGACTGCGCCCTGGACCCTCTGA
GCGAGACAAAGTGCACCCTGAAGTCCTTCACCGTGGAGAAGGGCATCTAC
CAGACCAGCAACTTCAGGGTGCAGCCCACCGAGAGCATCGTGAGGTTCCC
CAACATCACCAACCTGTGCCCCTTCGGCGAGGTGTTCAACGCCACCAGGT
TCGCCAGCGTGTACGCCTGGAACAGGAAGAGGATCAGCAACTGCGTGGCC
GACTACAGCGTGCTGTATAACAGCGCCAGCTTCAGCACCTTCAAGTGCTA
CGGCGTGAGCCCCACCAAGCTGAACGACCTGTGCTTCACCAACGTGTACG
CCGACAGCTTCGTGATCAGGGGCGACGAGGTGAGGCAGATCGCCCCTGGC
CAGACCGGCAAGATCGCCGACTACAACTACAAGCTGCCCGACGACTTCAC
CGGCTGCGTGATCGCCTGGAACAGCAACAACCTGGACAGCAAGGTGGGCG
GCAACTACAACTACCTGTACCGGCTGTTCAGAAAGAGCAACCTGAAGCCC
TTCGAGAGGGACATCAGCACCGAGATCTACCAGGCCGGCAGCACCCCTTG
CAACGGCGTGGAGGGCTTCAACTGCTACTTCCCTCTGCAGAGCTACGGCT
TCCAGCCCACCtACGGCGTGGGCTACCAGCCCTACAGGGTGGTGGTCCTG
AGCTTCGAGCTGCTGCACGCCCCTGCCACCGTGTGCGGCCCCAAGAAGTC
CACCAACCTGGTGAAGAACAAGTGCGTGAACTTCAACTTCAACGGCCTGA
CCGGCACCGGCGTGCTGACCGAGAGCAACAAGAAGTTCCTGCCCTTCCAG
CAGTTCGGCAGGGACATCGaCGACACCACCGACGCCGTGAGGGACCCTCA
GACCCTGGAGATCCTGGACATCACCCCTTGCAGCTTCGGCGGCGTGAGCG
TGATCACCCCTGGCACCAACACCAGCAACCAGGTGGCCGTGCTGTACCAG
GgCGTGAACTGCACCGAGGTGCCCGTGGCCATCCACGCCGACCAGCTGAC
CCCTACCTGGAGGGTGTACTCCACCGGCAGCAACGTGTTCCAGACCAGGG
CCGGCTGCCTGATCGGCGCCGAGCACGTGAACAACAGCTACGAGTGCGAC
ATCCCCATCGGCGCCGGCATCTGCGCCAGCTACCAGACCCAGACCAACAG
CCaCgGGaGcGCCAGcAGCGTGGCCAGCCAGAGCATCATCGCCTACACCA
TGAGCCTGGGCGCCGAGAACAGCGTGGCCTACAGCAACAACAGCATCGCC
ATCCCCAtCAACTTCACCATCAGCGTGACCACCGAGATCCTGCCCGTGAG
CATGACCAAGACCAGCGTGGACTGCACCATGTATATCTGCGGCGACAGCA
CCGAGTGCAGCAACCTGCTGCTCCAGTACGGCAGCTTCTGCACCCAGCTG
AACAGGGCCCTGACCGGCATCGCCGTGGAGCAGGACAAGAACACCCAGGA
GGTGTTCGCCCAGGTGAAGCAGATCTACAAGACCCCTCCCATCAAGGACT
TCGGCGGCTTCAACTTCAGCCAGATCCTGCCCGACCCCAGCAAGCCCAGC
AAGAGGAGCTTCATCGAGGACCTGCTGTTCAACAAGGTGACCCTGGCCGA
CGCCGGCTTCATCAAGCAGTACGGCGACTGCCTGGGCGACATCGCCGCCA
GGGACCTGATCTGCGCCCAGAAGTTCAACGGCCTGACCGTGCTGCCTCCC
CTGCTGACCGACGAGATGATCGCCCAGTACACCAGCGCCCTGCTGGCCGG
CACCATCACCAGCGGCTGGACCTTCGGCGCCGGCGCCGCCCTGCAGATCC
CCTTCGCCATGCAGATGGCCTACAGGTTCAACGGCATCGGCGTGACCCAG
AACGTGCTGTACGAGAACCAGAAGCTGATCGCCAACCAGTTCAACAGCGC
CATCGGCAAGATCCAGGACAGCCTGAGCAGCACCGCCAGCGCCCTGGGCA
AGCTGCAGGACGTGGTGAACCAGAACGCCCAGGCCCTGAACACCCTGGTG
AAGCAGCTGAGCAGCAACTTCGGCGCCATCAGCAGCGTGCTGAACGACAT
CCTGgcCAGGCTGGACccacccGAGGCCGAGGTGCAGATCGACAGGCTGA
TCACCGGCAGGCTGCAGAGCCTGCAGACCTACGTGACCCAGCAGCTGATC
AGGGCCGCCGAGATCAGGGCCAGCGCCAACCTGGCCGCCACCAAGATGAG
CGAGTGCGTGCTGGGCCAGAGCAAGAGGGTGGACTTCTGCGGCAAGGGCT
ACCACCTGATGAGCTTCCCTCAGAGCGCCCCTCACGGCGTGGTGTTCCTG
CACGTGACCTACGTGCCCGCCCAGGAGAAGAACTTCACCACAGCCCCTGC
CATCTGCCACGACGGCAAGGCCCACTTCCCCAGGGAGGGCGTGTTCGTGA
GCAACGGCACCCACTGGTTCGTGACCCAGAGGAACTTCTACGAGCCCCAG
ATCATCACCACCcACAACACCTTCGTGAGCGGCAACTGCGACGTGGTGAT
CGGCATCGTGAACAACACCGTGTACGACCCTCTGCAGCCCGAGCTGGACA
GCTTCAAGGAGGAGCTGGACAAGTACTTCAAGAACCACACCAGCCCCGAC
GTGGACCTGGGCGACATCAGCGGCATCAACGCCAGCGTGGTGAACATCCA
GAAGGAGATCGACAGGCTGAACGAGGTGGCCAAGAACCTGAACGAGAGCC
TGATCGACCTGCAGGAGCTGGGCAAGTACGAGCAGTACATCAAGTGGCCC
TGGTACATCTGGCTGGGCTTCATCGCCGGCCTGATCGCCATCGTGATGGT
GACCATCATGCTGTGCTGCATGACCAGCTGCTGCAGCTGCCTGAAGGGCT
GCTGCAGCTGCGGCAGCTGCTGCAAGTTCGACGAGGACGACAGCGAGCCC
GTGCTGAAGGGCGTGAAGCTGCACTACACCTaAacTCGAGTATGTTACGT
GCAAAGGTGATTGTCACCCCCCGAAAGACCATATTGTGACACACCCTCAG
TATCACGCCCAAACATTTACAGCCGCGGTGTCAAAAACCGCGTGGACGTG
GTTAACATCCCTGCTGGGAGGATCAGCCGTAATTATTATAATTGGCTTGG
TGCTGGCTACTATTGTGGCCATGTACGTGCTGACCAACCAGAAACATAAT
TGAATACAGCAGCAATTGGCAAGCTGCTTACATAGAACTCGCGGCGATTG
GCATGCCGCCTTAAAATTTTTATTTTATTTTTTCTTTTCTTTTCCGAATC
GGATTTTGTTTTTAATATTTCAAAAAAAAAAAAAAAAAAAAAAAAATcta
gAAAAAAAAAAAAAAAAAAAAAAAAAAAAAAAAAAAAAAAAAAAAAAAAA
AAAAAAAAAAAAAAAAAAAAAAAAAAAAAAAaaaaaaaaaaaaaaaaaaa
a

SEQ ID NO:4 - mARM3346 (Brazil P.1)

atgggcggcgcatgagagaagcccagaccaattacctacccaaaatggag
aaagttcacgttgacatcgaggaagacagcccattcctcagagctttgca
gcggagcttcccgcagtttgaggtagaagccaagcaggtcactgataatg
accatgctaatgccagagcgttttcgcatctggcttcaaaactgatcgaa
acggaggtggacccatccgacacgatccttgacattggaagtgcgcccgc
ccgcagaatGTATTCTAAGCACAAGTATCATTGTATCtgtccgatgagat
gtgcggaagatccggacagattgtataagtatgcaactaagctgaagaaa
aactgtaaggaaataactgataaggaattggacaagaaaatgaaggagct
ggccgccgtcatgagcgaccctgacctggaaactgagactatgtgcctcc
acgacgacgagtcgtgtcgctacgaagggcaagtcgctgtttaccaggat
gtatacgcCGTcGACGGCCCCACCAGCCTGTACCACCAGGCCAACAAGGG
CGTGAGGGTGGCCTACTGGATCGGCTTCGACACCACACCCTTCATGTTCA
AGAACCTGGCCGGCGCCTACCCCAGCTACAGCACCAACTGGGCCGACGAG
ACAGTGCTGACCGCCAGGAACATCGGCCTGTGCAGCAGCGACGTGATGGA
GAGGAGCCGGAGGGGCATGAGCATCCTGAGGAAGAAGTACCTGAAGCCCA
GCAACAACGTGCTGTTCAGCGTGGGCAGCACCATCTACCACGAGAAGAGG
GACCTGCTGAGGAGCTGGCACCTGCCCAGCGTGTTCCACCTGAGGGGCAA
GCAGAACTACACCTGCAGGTGCGAGACAATCGTGAGCTGCGACGGCTACG
TGGTGAAGAGGATCGCCATCAGCCCCGGCCTGTACGGCAAGCCCAGCGGC
TACGCCGCCACCATGCACAGGGAGGGCTTCCTGTGCTGCAAGGTGACCGA
CACCCTGAACGGCGAGAGGGTGAGCTTCCCCGTGTGCACCTACGTGCCCG
CCACCCTGTGCGACCAGATGACCGGCATCCTGGCCACCGACGTGAGCGCC
GACGACGCCCAGAAGCTGCTGGTGGGCCTGAACCAGAGGATCGTGGTGAA
CGGCAGGACCCAGAGGAACACCAACACCATGAAGAACTACCTGCTGCCCG
TGGTGGCCCAGGCCTTCGCCAGGTGGGCCAAGGAGTACAAGGAGGACCAG
GAGGACGAGAGGCCCCTGGGCCTGAGGGACcGaCAGCTGGTGATGGGCTG
CTGCTGGGCCTTCAGGCGGCACAAGATCACCAGCATCTACAAGAGGCCCG
ACACCCAGACCATCATCAAGGTGAACAGCGACTTCCACAGCTTCGTGCTG
CCCAGGATCGGCAGCAACACCCTGGAGATCGGCCTGAGGACCCGGATCAG
GAAGATGCTGGAGGAGCACAAGGAGCCCAGCCCTCTGATCACCGCCGAGG
ACGTGCAGGAGGCCAAGTGCGCCGCCGACGAGGCCAAGGAGGTGAGGGAG
GCCGAGGAGCTGAGGGCCGCCCTGCCTCCCCTGGCCGCCGACGTGGAGGA
GCCCACCCTGGAGGCCGACGTGGACCTGATGCTGCAGGAGGCCGGCGCCG
GCAGCGTGGAGACACCCAGGGGCCTGATCAAGGTGACCAGCTACGACGGC
GAGGACAAGATCGGCAGCTACGCCGTGCTcAGCCCTCAGGCCGTGCTGAA
GTCCGAGAAGCTGAGCTGCATCCACCCTCTGGCCGAGCAGGTGATCGTGA
TCACCCACAGCGGCAGGAAGGGCAGGTACGCCGTGGAGCCCTACCACGGC
AAGGTGGTGGTCCCCGAGGGCCACGCCATCCCCGTGCAGGACTTCCAGGC
CCTGAGCGAGAGCGCCACCATCGTGTATAACGAGAGGGAGTTCGTGAACA
GGTACCTGCACCACATCGCCACCCACGGCGGCGCCCTGAACACCGACGAG
GAGTACTACAAGACCGTGAAGCCCAGCGAGCACGACGGCGAGTACCTGTA
CGACATCGACAGGAAGCAGTGCGTGAAGAAGGAGCTGGTGACCGGCCTGG
GCCTGACCGGCGAGCTGGTGGACCCTCCCTTCCACGAGTTCGCCTACGAG
AGCCTGAGGACCAGGCCCGCCGCTCCCTACCAGGTGCCCACCATCGGCGT
GTACGGCGTGCCCGGCAGCGGCAAGAGCGGCATCATCAAGAGCGCCGTGA
CCAAGAAGGACCTGGTGGTGAGCGCCAAGAAGGAGAACTGCGCCGAGATC
ATCAGGGACGTGAAGAAGATGAAGGGCCTGGACGTGAACGCCAGGACCGT
GGACAGCGTGCTcCTGAACGGCTGCAAGCACCCCGTGGAGACACTGTATA
TCGACGAGGCCTTCGCCTGCCACGCCGGCACCCTGAGGGCCCTGATCGCC
ATCATCAGGCCCAAGAAGGCCGTGCTGTGCGGCGACCCCAAGCAGTGCGG
CTTCTTCAACATGATGTGCCTGAAGGTGCACTTCAACCACGAGATCTGCA
CCCAGGTGTTCCACAAGAGCATCAGCAGGCGGTGCACCAAGAGCGTGACC
AGCGTGGTGAGCACCCTGTTCTACGACAAGAAGATGAGGACCACCAACCC
CAAGGAGACAAAGATCGTGATCGACACCACCGGCAGCACCAAGCCCAAGC
AGGACGACCTGATCCTGACCTGCTTCAGGGGCTGGGTGAAGCAGCTGCAG
ATCGACTACAAGGGCAACGAGATCATGACCGCCGCCGCTAGCCAGGGCCT
GACCAGGAAGGGCGTGTACGCCGTGAGGTACAAGGTGAACGAGAATCCCC
TGTACGCCCCTACCAGCGAGCACGTGAACGTcCTGCTGACCAGGACCGAG
GACAGGATCGTGTGGAAGACCCTGGCCGGCGACCCCTGGATCAAGACCCT
GACCGCCAAGTACCCCGGCAACTTCACCGCCACCATCGAGGAGTGGCAGG
CCGAGCACGACGCCATCATGAGGCACATCCTGGAGAGGCCCGACCCCACC
GACGTGTTCCAGAACAAGGCCAACGTGTGCTGGGCCAAGGCCCTGGTGCC
CGTGCTGAAGACCGCCGGCATCGACATGACCACCGAGCAGTGGAACACCG
TGGACTACTTCGAGACAGACAAGGCCCACAGCGCCGAGATCGTGCTGAAC
CAGCTGTGCGTGAGGTTCTTCGGCCTGGACCTGGACAGCGGCCTGTTCAG
CGCCCCTACCGTGCCCCTGAGCATCAGGAACAACCACTGGGACAACAGCC
CCAGCCCCAACATGTACGGCCTGAACAAGGAGGTGGTGAGGCAGCTGAGC
AGGCGGTACCCTCAGCTGCCCAGGGCCGTGGCCACCGGCAGGGTGTACGA
CATGAACACCGGCACCCTGAGGAACTACGACCCCAGGATCAACCTGGTGC
CCGTGAACAGGCGGCTGCCaCACGCCCTGGTGCTGCACCACAACGAGCAC
CCTCAGAGCGACTTCAGCAGCTTCGTGAGCAAGCTGAAGGGCAGGACCGT
GCTGGTGGTGGGCGAGAAGCTGAGCGTGCCCGGCAAGATGGTGGACTGGC
TGAGCGACAGGCCCGAGGCCACCTTCCGGGCCAGGCTGGACCTGGGCATC
CCCGGCGACGTGCCCAAGTACGACATCATCTTCGTGAACGTGAGGACCCC
TTACAAGTACCACCACTACCAGCAGTGCGAGGACCACGCCATCAAGCTGA
GCATGCTGACCAAGAAGGCCTGCCTGCACCTGAACCCCGGCGGCACCTGC
GTGAGCATCGGCTACGGCTACGCCGACAGGGCCAGCGAGAGCATCATCGG
CGCCATCGCCAGGCTGTTCAAGTTCAGCAGGGTGTGCAAGCCCAAGAGCA
GCCTGGAGGAGACAGAGGTGCTGTTCGTGTTCATCGGCTACGACCGGAAG
GCCAGGACCCACAACCCCTACAAGCTGAGCAGCACCCTGACCAACATCTA
CACCGGCAGCAGGCTGCACGAGGCCGGCTGCGCCCCTAGCTACCACGTGG
TGAGGGGCGACATCGCCACCGCCACCGAGGGCGTGATCATCAACGCCGCC
AACAGCAAGGGCCAGCCCGGCGGCGGGGTGTGCGGCGCCCTGTATAAGAA
GTTCCCCGAGAGCTTCGACCTGCAGCCCATCGAGGTGGGCAAGGCCAGGC
TGGTGAAGGGCGCCGCCAAGCACATCATCCACGCCGTGGGCCCCAACTTC
AACAAGGTGAGCGAGGTGGAGGGCGACAAGCAGCTGGCCGAGGCCTACGA
GAGCATCGCCAAGATCGTGAACGACAACAACTACAAGAGCGTGGCCATCC
CTCTGCTGAGCACCGGCATCTTCAGCGGCAACAAGGACAGGCTGACCCAG
AGCCTGAACCACCTGCTGACCGCCCTGGACACCACCGACGCCGACGTGGC
CATCTACTGCAGGGACAAGAAGTGGGAGATGACCCTGAAGGAGGCCGTGG
CCAGGCGGGAGGCCGTGGAGGAGATCTGCATCAGCGACGACAGCAGCGTG
ACgGAGCCCGACGCCGAGCTGGTGAGGGTGCACCCCAAGAGCAGCCTGGC
CGGCAGGAAGGGCTACAGCACCAGCGACGGCAAGACCTTCAGCTACCTGG
AGGGCACCAAGTTCCACCAGGCCGCCAAGGACATCGCCGAGATCAACGCC
ATGTGGCCCGTGGCCACCGAGGCCAACGAGCAGGTGTGCATGTATATCCT
GGGCGAGAGCATGAGCAGCATCAGGAGCAAGTGCCCCGTGGAGGAGAGCG
AGGCCAGCACCCCTCCCAGCACCCTGCCCTGCCTGTGCATCCACGCCATG
ACCCCTGAGAGGGTGCAGCGGCTGAAGGCCAGCAGGCCCGAGCAGATCAC
CGTGTGCAGCAGCTTCCCTCTGCCCAAGTACcGGATCACCGGCGTGCAGA
AGATCCAGTGCAGCCAGCCCATCCTGTTCAGCCCCAAGGTGCCCGCCTAC
ATCCACCCCAGGAAGTACCTGGTGGAGACACCCCCCGTGGACGAGACACC
CGAGCCCAGCGCCGAGAACCAGAGCACCGAGGGCACCCCTGAGCAGCCTC
CCCTGATCACCGAGGACGAGACAAGGACCAGGACgCCcGAGCCCATCATC
ATTGAGGAGGAAGAGGAGGACAGCATCAGCCTGCTGAGCGACGGCCCCAC
CCACCAGGTGCTGCAGGTGGAGGCCGACATCCACGGCCCTCCCAGCGTGA
GCAGCTCCAGCTGGAGCATCCCTCACGCCAGCGACTTCGACGTGGACAGC
CTGAGCATCCTGGACACCCTGGAGGGCGCCAGCGTGACCAGCGGCGCCAC
CAGCGCCGAGACAAACAGCTACTTCGCCAAGAGCATGGAGTTCCTGGCCA
GGCCCGTGCCCGCCCCTAGGACCGTGTTCAGGAACCCTCCCCACCCCGCC
CCTAGGACCAGGACCCCTAGCCTGGCCCCTAGCAGGGCCTGCAGCAGGAC
CAGCCTGGTGAGCACCCCTCCCGGCGTGAACcGGGTGATCACCAGGGAGG
AGCTGGAGGCCCTGACCCCTAGCAGGACCCCTAGCAGGAGCGTGAGCAGG
ACCAGCCTGGTGAGCAACCCTCCCGGCGTGAACcGGGTGATCACCAGGGA
GGAGTTCGAGGCCTTCGTGGCCCAGCAGCAAAGGCGGTTCGACGCCGGCG
CCTACATCTTCAGCAGCGACACCGGCCAGGGCCACCTGCAGCAGAAGTCC
GTGAGGCAGACCGTGCTGAGCGAGGTGGTcCTGGAGAGGACgGAGCTGGA
GATCAGCTACGCCCCTAGGCTGGACCAGGAGAAGGAGGAGCTGCTGAGGA
AGAAGCTGCAGCTGAACCCCACCCCTGCCAACAGGAGCAGGTACCAGAGC
AGGAAGGTGGAGAACATGAAGGCCATCACCGCCAGGCGGATCCTGCAGGG
CCTGGGCCACTACCTGAAGGCCGAGGGCAAGGTGGAGTGCTACAGGACCC
TGCACCCCGTGCCCCTGTACTCCAGCTCCGTGAACAGGGCCTTCAGCAGC
CCCAAGGTGGCCGTGGAGGCCTGCAACGCCATGCTGAAGGAGAACTTCCC
CACCGTGGCCAGCTACTGCATCATCCCCGAGTACGACGCCTACCTGGACA
TGGTGGACGGCGCCAGCTGCTGCCTGGACACCGCCAGCTTCTGCCCCGCC
AAGCTGAGGAGCTTCCCCAAGAAGCACAGCTACCTGGAGCCCACCATCAG
GAGCGCCGTGCCCAGCGCCATCCAGAACACCCTGCAGAACGTGCTGGCCG
CCGCTACCAAGAGGAACTGCAACGTGACCCAGATGAGGGAGCTGCCCGTG
CTGGACAGCGCCGCCTTCAACGTGGAGTGCTTCAAGAAGTACGCCTGCAA
CAACGAGTACTGGGAGACATTCAAGGAGAACCCCATCAGGCTGACCGAGG
AGAACGTGGTGAACTACATCACCAAGCTGAAGGGCCCCAAGGCCGCCGCT
CTGTTCGCCAAGACCCACAACCTGAACATGCTcCAGGACATCCCTATGGA
CAGGTTCGTGATGGACCTGAAGAGGGACGTGAAGGTGACCCCTGGCACCA
AGCACACCGAGGAGAGGCCCAAGGTGCAGGTGATCCAGGCCGCCGACCCT
CTGGCCACCGCCTACCTGTGCGGCATCCACAGGGAGCTGGTGAGGCGGCT
GAACGCCGTcCTGCTGCCCAACATCCACACCCTGTTCGACATGAGCGCCG
AGGACTTCGACGCCATCATCGCCGAGCACTTCCAGCCCGGCGACTGCGTG
CTGGAGACAGACATCGCCAGCTTCGACAAGAGCGAGGACGACGCTATGGC
CCTGACCGCCCTGATGATCCTGGAGGACCTGGGCGTGGACGCCGAGCTGC
TGACCCTGATCGAGGCCGCCTTCGGCGAGATCAGCAGCATCCACCTGCCC
ACCAAGACCAAGTTCAAGTTCGGCGCCATGATGAAGTCCGGCATGTTCCT
GACCCTGTTCGTGAACACCGTGATCAACATCGTGATCGCCAGCAGGGTGC
TGCGGGAGAGGCTGACCGGCAGCCCCTGCGCCGCCTTCATCGGCGACGAC
AACATCGTGAAGGGCGTGAAGTCCGACAAGCTGATGGCCGACAGGTGCGC
CACCTGGCTGAACATGGAGGTGAAGATCATCGACGCCGTGGTGGGCGAGA
AGGCCCCTTACTTCTGCGGCGGCTTCATCCTGTGCGACAGCGTGACCGGC
ACCGCCTGCAGGGTGGCCGACCCTCTGAAGAGGCTGTTCAAGCTGGGCAA
GCCCCTGGCCGCCGACGACGAGCACGACGACGATAGGCGGAGGGCCCTGC
ACGAGGAGAGCACCAGGTGGAACcGGGTGGGCATCCTGAGCGAGCTGTGC
AAGGCCGTGGAGAGCAGGTACGAGACAGTGGGCACCAGCATCATCGTGAT
GGCCATGACCACCCTGGCCAGCAGCGTcAAGTCCTTCAGCTACCTGAGGG
GGGCCCCTATAACTCTCTACGGCTAACCTGAATGGACTACGACATAGTCT
AGTCCGCCAAGGCCGCCACCATGTTCGTGTTCCTGGTGCTGCTGCCCCTG
GTGTCTAGCCAGTGCGTGAACtTcACCAaCAGGACCCAGCTGCCTagCGC
CTACACCAACAGCTTCACCAGGGGCGTGTACTACCCCGACAAGGTGTTCA
GGAGCAGCGTGCTGCACAGCACCCAGGACCTGTTCCTGCCCTTCTTCAGC
AACGTGACCTGGTTCCACGCCATCCACGTGAGCGGCACCAACGGCACCAA
GAGGTTCGACAACCCCGTGCTGCCCTTCAACGACGGCGTGTACTTCGCCA
GCACCGAGAAGTCCAACATCATCAGGGGCTGGATCTTCGGCACCACCCTG
GACAGCAAGACCCAGAGCCTGCTGATCGTGAACAACGCCACCAACGTGGT
GATCAAGGTGTGCGAGTTCCAGTTCTGCAACtACCCCTTCCTGGGCGTGT
ACTACCACAAGAACAACAAGAGCTGGATGGAGAGCGAGTTCAGGGTGTAC
TCCAGCGCCAACAACTGCACCTTCGAGTACGTGAGCCAGCCCTTCCTGAT
GGACCTGGAGGGCAAGCAGGGCAACTTCAAGAACCTGAGcGAGTTCGTGT
TCAAGAACATCGACGGCTACTTCAAGATCTACAGCAAGCACACCCCTATC
AACCTGGTGAGGGACCTGCCCCAGGGCTTCAGCGCCCTGGAGCCCCTGGT
GGACCTGCCCATCGGCATCAACATCACCAGGTTCCAGACCCTGCTGGCCC
TGCACAGGAGCTACCTGACCCCTGGCGACAGCAGCTCCGGCTGGACCGCC
GGCGCCGCCGCTTACTACGTGGGCTACCTGCAGCCCAGGACCTTCCTGCT
GAAGTACAACGAGAACGGCACCATCACCGACGCCGTGGACTGCGCCCTGG
ACCCTCTGAGCGAGACAAAGTGCACCCTGAAGTCCTTCACCGTGGAGAAG
GGCATCTACCAGACCAGCAACTTCAGGGTGCAGCCCACCGAGAGCATCGT
GAGGTTCCCCAACATCACCAACCTGTGCCCCTTCGGCGAGGTGTTCAACG
CCACCAGGTTCGCCAGCGTGTACGCCTGGAACAGGAAGAGGATCAGCAAC
TGCGTGGCCGACTACAGCGTGCTGTATAACAGCGCCAGCTTCAGCACCTT
CAAGTGCTACGGCGTGAGCCCCACCAAGCTGAACGACCTGTGCTTCACCA
ACGTGTACGCCGACAGCTTCGTGATCAGGGGCGACGAGGTGAGGCAGATC
GCCCCTGGCCAGACCGGCAccATCGCCGACTACAACTACAAGCTGCCCGA
CGACTTCACCGGCTGCGTGATCGCCTGGAACAGCAACAACCTGGACAGCA
AGGTGGGCGGCAACTACAACTACCTGTACCGGCTGTTCAGAAAGAGCAAC
CTGAAGCCCTTCGAGAGGGACATCAGCACCGAGATCTACCAGGCCGGCAG
CACCCCTTGCAACGGCGTGaAGGGCTTCAACTGCTACTTCCCTCTGCAGA
GCTACGGCTTCCAGCCCACCtACGGCGTGGGCTACCAGCCCTACAGGGTG
GTGGTCCTGAGCTTCGAGCTGCTGCACGCCCCTGCCACCGTGTGCGGCCC
CAAGAAGTCCACCAACCTGGTGAAGAACAAGTGCGTGAACTTCAACTTCA
ACGGCCTGACCGGCACCGGCGTGCTGACCGAGAGCAACAAGAAGTTCCTG
CCCTTCCAGCAGTTCGGCAGGGACATCGCCGACACCACCGACGCCGTGAG
GGACCCTCAGACCCTGGAGATCCTGGACATCACCCCTTGCAGCTTCGGCG
GCGTGAGCGTGATCACCCCTGGCACCAACACCAGCAACCAGGTGGCCGTG
CTGTACCAGggcGTGAACTGCACCGAGGTGCCCGTGGCCATCCACGCCGA
CCAGCTGACCCCTACCTGGAGGGTGTACTCCACCGGCAGCAACGTGTTCC
AGACCAGGGCCGGCTGCCTGATCGGCGCCGAGtACGTGAACAACAGCTAC
GAGTGCGACATCCCCATCGGCGCCGGCATCTGCGCCAGCTACCAGACCCA
GACCAACAGCCCCgGGaGcGCCAGcAGCGTGGCCAGCCAGAGCATCATCG
CCTACACCATGAGCCTGGGCGCCGAGAACAGCGTGGCCTACAGCAACAAC
AGCATCGCCATCCCCACCAACTTCACCATCAGCGTGACCACCGAGATCCT
GCCCGTGAGCATGACCAAGACCAGCGTGGACTGCACCATGTATATCTGCG
GCGACAGCACCGAGTGCAGCAACCTGCTGCTCCAGTACGGCAGCTTCTGC
ACCCAGCTGAACAGGGCCCTGACCGGCATCGCCGTGGAGCAGGACAAGAA
CACCCAGGAGGTGTTCGCCCAGGTGAAGCAGATCTACAAGACCCCTCCCA
TCAAGGACTTCGGCGGCTTCAACTTCAGCCAGATCCTGCCCGACCCCAGC
AAGCCCAGCAAGAGGAGCTTCATCGAGGACCTGCTGTTCAACAAGGTGAC
CCTGGCCGACGCCGGCTTCATCAAGCAGTACGGCGACTGCCTGGGCGACA
TCGCCGCCAGGGACCTGATCTGCGCCCAGAAGTTCAACGGCCTGACCGTG
CTGCCTCCCCTGCTGACCGACGAGATGATCGCCCAGTACACCAGCGCCCT
GCTGGCCGGCACCATCACCAGCGGCTGGACCTTCGGCGCCGGCGCCGCCC
TGCAGATCCCCTTCGCCATGCAGATGGCCTACAGGTTCAACGGCATCGGC
GTGACCCAGAACGTGCTGTACGAGAACCAGAAGCTGATCGCCAACCAGTT
CAACAGCGCCATCGGCAAGATCCAGGACAGCCTGAGCAGCACCGCCAGCG
CCCTGGGCAAGCTGCAGGACGTGGTGAACCAGAACGCCCAGGCCCTGAAC
ACCCTGGTGAAGCAGCTGAGCAGCAACTTCGGCGCCATCAGCAGCGTGCT
GAACGACATCCTGAGCAGGCTGGACccacccGAGGCCGAGGTGCAGATCG
ACAGGCTGATCACCGGCAGGCTGCAGAGCCTGCAGACCTACGTGACCCAG
CAGCTGATCAGGGCCGCCGAGATCAGGGCCAGCGCCAACCTGGCCGCCAt
CAAGATGAGCGAGTGCGTGCTGGGCCAGAGCAAGAGGGTGGACTTCTGCG
GCAAGGGCTACCACCTGATGAGCTTCCCTCAGAGCGCCCCTCACGGCGTG
GTGTTCCTGCACGTGACCTACGTGCCCGCCCAGGAGAAGAACTTCACCAC
AGCCCCTGCCATCTGCCACGACGGCAAGGCCCACTTCCCCAGGGAGGGCG
TGTTCGTGAGCAACGGCACCCACTGGTTCGTGACCCAGAGGAACTTCTAC
GAGCCCCAGATCATCACCACCGACAACACCTTCGTGAGCGGCAACTGCGA
CGTGGTGATCGGCATCGTGAACAACACCGTGTACGACCCTCTGCAGCCCG
AGCTGGACAGCTTCAAGGAGGAGCTGGACAAGTACTTCAAGAACCACACC
AGCCCCGACGTGGACCTGGGCGACATCAGCGGCATCAACGCCAGCGTGGT
GAACATCCAGAAGGAGATCGACAGGCTGAACGAGGTGGCCAAGAACCTGA
ACGAGAGCCTGATCGACCTGCAGGAGCTGGGCAAGTACGAGCAGTACATC
AAGTGGCCCTGGTACATCTGGCTGGGCTTCATCGCCGGCCTGATCGCCAT
CGTGATGGTGACCATCATGCTGTGCTGCATGACCAGCTGCTGCAGCTGCC
TGAAGGGCTGCTGCAGCTGCGGCAGCTGCTGCAAGTTCGACGAGGACGAC
AGCGAGCCCGTGCTGAAGGGCGTGAAGCTGCACTACACCTaAaCTCGAGT
ATGTTACGTGCAAAGGTGATTGTCACCCCCCGAAAGACCATATTGTGACA
CACCCTCAGTATCACGCCCAAACATTTACAGCCGCGGTGTCAAAAACCGC
GTGGACGTGGTTAACATCCCTGCTGGGAGGATCAGCCGTAATTATTATAA
TTGGCTTGGTGCTGGCTACTATTGTGGCCATGTACGTGCTGACCAACCAG
AAACATAATTGAATACAGCAGCAATTGGCAAGCTGCTTACATAGAACTCG
CGGCGATTGGCATGCCGCCTTAAAATTTTTATTTTATTTTTTCTTTTCTT
TTCCGAATCGGATTTTGTTTTTAATATTTCAAAAAAAAAAAAAAAAAAAA
AAAAATctagAAAAAAAAAAAAAAAAAAAAAAAAAAAAAAAAAAAAAAAA
AAAAAAAAAAAAAAAAAAAAAAAAAAAAAAAAAAAAAAAAaaaaaaaaaa
aaaaaaaaaa

SEQ ID NO:5 - 5′ UTR (of SEQ ID NOs: 1-4)

ATGGGCGGCGCATGAGAGAAGCCCAGACCAATTACCTACCCAAA

SEQ ID NO:6 - nsP1-nsP4 (of SEQ ID NOs:1-4)

ATGGAGAAAGTTCACGTTGACATCGAGGAAGACAGCCCATTCCTCAGAGC
TTTGCAGCGGAGCTTCCCGCAGTTTGAGGTAGAAGCCAAGCAGGTCACTG
ATAATGACCATGCTAATGCCAGAGCGTTTTCGCATCTGGCTTCAAAACTG
ATCGAAACGGAGGTGGACCCATCCGACACGATCCTTGACATTGGAAGTGC
GCCCGCCCGCAGAATGTATTCTAAGCACAAGTATCATTGTATCTGTCCGA
TGAGATGTGCGGAAGATCCGGACAGATTGTATAAGTATGCAACTAAGCTG
AAGAAAAACTGTAAGGAAATAACTGATAAGGAATTGGACAAGAAAATGAA
GGAGCTGGCCGCCGTCATGAGCGACCCTGACCTGGAAACTGAGACTATGT
GCCTCCACGACGACGAGTCGTGTCGCTACGAAGGGCAAGTCGCTGTTTAC
CAGGATGTATACGCCGTCGACGGCCCCACCAGCCTGTACCACCAGGCCAA
CAAGGGCGTGAGGGTGGCCTACTGGATCGGCTTCGACACCACACCCTTCA
TGTTCAAGAACCTGGCCGGCGCCTACCCCAGCTACAGCACCAACTGGGCC
GACGAGACAGTGCTGACCGCCAGGAACATCGGCCTGTGCAGCAGCGACGT
GATGGAGAGGAGCCGGAGGGGCATGAGCATCCTGAGGAAGAAGTACCTGA
AGCCCAGCAACAACGTGCTGTTCAGCGTGGGCAGCACCATCTACCACGAG
AAGAGGGACCTGCTGAGGAGCTGGCACCTGCCCAGCGTGTTCCACCTGAG
GGGCAAGCAGAACTACACCTGCAGGTGCGAGACAATCGTGAGCTGCGACG
GCTACGTGGTGAAGAGGATCGCCATCAGCCCCGGCCTGTACGGCAAGCCC
AGCGGCTACGCCGCCACCATGCACAGGGAGGGCTTCCTGTGCTGCAAGGT
GACCGACACCCTGAACGGCGAGAGGGTGAGCTTCCCCGTGTGCACCTACG
TGCCCGCCACCCTGTGCGACCAGATGACCGGCATCCTGGCCACCGACGTG
AGCGCCGACGACGCCCAGAAGCTGCTGGTGGGCCTGAACCAGAGGATCGT
GGTGAACGGCAGGACCCAGAGGAACACCAACACCATGAAGAACTACCTGC
TGCCCGTGGTGGCCCAGGCCTTCGCCAGGTGGGCCAAGGAGTACAAGGAG
GACCAGGAGGACGAGAGGCCCCTGGGCCTGAGGGACCGACAGCTGGTGAT
GGGCTGCTGCTGGGCCTTCAGGCGGCACAAGATCACCAGCATCTACAAGA
GGCCCGACACCCAGACCATCATCAAGGTGAACAGCGACTTCCACAGCTTC
GTGCTGCCCAGGATCGGCAGCAACACCCTGGAGATCGGCCTGAGGACCCG
GATCAGGAAGATGCTGGAGGAGCACAAGGAGCCCAGCCCTCTGATCACCG
CCGAGGACGTGCAGGAGGCCAAGTGCGCCGCCGACGAGGCCAAGGAGGTG
AGGGAGGCCGAGGAGCTGAGGGCCGCCCTGCCTCCCCTGGCCGCCGACGT
GGAGGAGCCCACCCTGGAGGCCGACGTGGACCTGATGCTGCAGGAGGCCG
GCGCCGGCAGCGTGGAGACACCCAGGGGCCTGATCAAGGTGACCAGCTAC
GACGGCGAGGACAAGATCGGCAGCTACGCCGTGCTCAGCCCTCAGGCCGT
GCTGAAGTCCGAGAAGCTGAGCTGCATCCACCCTCTGGCCGAGCAGGTGA
TCGTGATCACCCACAGCGGCAGGAAGGGCAGGTACGCCGTGGAGCCCTAC
CACGGCAAGGTGGTGGTCCCCGAGGGCCACGCCATCCCCGTGCAGGACTT
CCAGGCCCTGAGCGAGAGCGCCACCATCGTGTATAACGAGAGGGAGTTCG
TGAACAGGTACCTGCACCACATCGCCACCCACGGCGGCGCCCTGAACACC
GACGAGGAGTACTACAAGACCGTGAAGCCCAGCGAGCACGACGGCGAGTA
CCTGTACGACATCGACAGGAAGCAGTGCGTGAAGAAGGAGCTGGTGACCG
GCCTGGGCCTGACCGGCGAGCTGGTGGACCCTCCCTTCCACGAGTTCGCC
TACGAGAGCCTGAGGACCAGGCCCGCCGCTCCCTACCAGGTGCCCACCAT
CGGCGTGTACGGCGTGCCCGGCAGCGGCAAGAGCGGCATCATCAAGAGCG
CCGTGACCAAGAAGGACCTGGTGGTGAGCGCCAAGAAGGAGAACTGCGCC
GAGATCATCAGGGACGTGAAGAAGATGAAGGGCCTGGACGTGAACGCCAG
GACCGTGGACAGCGTGCTCCTGAACGGCTGCAAGCACCCCGTGGAGACAC
TGTATATCGACGAGGCCTTCGCCTGCCACGCCGGCACCCTGAGGGCCCTG
ATCGCCATCATCAGGCCCAAGAAGGCCGTGCTGTGCGGCGACCCCAAGCA
GTGCGGCTTCTTCAACATGATGTGCCTGAAGGTGCACTTCAACCACGAGA
TCTGCACCCAGGTGTTCCACAAGAGCATCAGCAGGCGGTGCACCAAGAGC
GTGACCAGCGTGGTGAGCACCCTGTTCTACGACAAGAAGATGAGGACCAC
CAACCCCAAGGAGACAAAGATCGTGATCGACACCACCGGCAGCACCAAGC
CCAAGCAGGACGACCTGATCCTGACCTGCTTCAGGGGCTGGGTGAAGCAG
CTGCAGATCGACTACAAGGGCAACGAGATCATGACCGCCGCCGCTAGCCA
GGGCCTGACCAGGAAGGGCGTGTACGCCGTGAGGTACAAGGTGAACGAGA
ATCCCCTGTACGCCCCTACCAGCGAGCACGTGAACGTCCTGCTGACCAGG
ACCGAGGACAGGATCGTGTGGAAGACCCTGGCCGGCGACCCCTGGATCAA
GACCCTGACCGCCAAGTACCCCGGCAACTTCACCGCCACCATCGAGGAGT
GGCAGGCCGAGCACGACGCCATCATGAGGCACATCCTGGAGAGGCCCGAC
CCCACCGACGTGTTCCAGAACAAGGCCAACGTGTGCTGGGCCAAGGCCCT
GGTGCCCGTGCTGAAGACCGCCGGCATCGACATGACCACCGAGCAGTGGA
ACACCGTGGACTACTTCGAGACAGACAAGGCCCACAGCGCCGAGATCGTG
CTGAACCAGCTGTGCGTGAGGTTCTTCGGCCTGGACCTGGACAGCGGCCT
GTTCAGCGCCCCTACCGTGCCCCTGAGCATCAGGAACAACCACTGGGACA
ACAGCCCCAGCCCCAACATGTACGGCCTGAACAAGGAGGTGGTGAGGCAG
CTGAGCAGGCGGTACCCTCAGCTGCCCAGGGCCGTGGCCACCGGCAGGGT
GTACGACATGAACACCGGCACCCTGAGGAACTACGACCCCAGGATCAACC
TGGTGCCCGTGAACAGGCGGCTGCCACACGCCCTGGTGCTGCACCACAAC
GAGCACCCTCAGAGCGACTTCAGCAGCTTCGTGAGCAAGCTGAAGGGCAG
GACCGTGCTGGTGGTGGGCGAGAAGCTGAGCGTGCCCGGCAAGATGGTGG
ACTGGCTGAGCGACAGGCCCGAGGCCACCTTCCGGGCCAGGCTGGACCTG
GGCATCCCCGGCGACGTGCCCAAGTACGACATCATCTTCGTGAACGTGAG
GACCCCTTACAAGTACCACCACTACCAGCAGTGCGAGGACCACGCCATCA
AGCTGAGCATGCTGACCAAGAAGGCCTGCCTGCACCTGAACCCCGGCGGC
ACCTGCGTGAGCATCGGCTACGGCTACGCCGACAGGGCCAGCGAGAGCAT
CATCGGCGCCATCGCCAGGCTGTTCAAGTTCAGCAGGGTGTGCAAGCCCA
AGAGCAGCCTGGAGGAGACAGAGGTGCTGTTCGTGTTCATCGGCTACGAC
CGGAAGGCCAGGACCCACAACCCCTACAAGCTGAGCAGCACCCTGACCAA
CATCTACACCGGCAGCAGGCTGCACGAGGCCGGCTGCGCCCCTAGCTACC
ACGTGGTGAGGGGCGACATCGCCACCGCCACCGAGGGCGTGATCATCAAC
GCCGCCAACAGCAAGGGCCAGCCCGGCGGCGGGGTGTGCGGCGCCCTGTA
TAAGAAGTTCCCCGAGAGCTTCGACCTGCAGCCCATCGAGGTGGGCAAGG
CCAGGCTGGTGAAGGGCGCCGCCAAGCACATCATCCACGCCGTGGGCCCC
AACTTCAACAAGGTGAGCGAGGTGGAGGGCGACAAGCAGCTGGCCGAGGC
CTACGAGAGCATCGCCAAGATCGTGAACGACAACAACTACAAGAGCGTGG
CCATCCCTCTGCTGAGCACCGGCATCTTCAGCGGCAACAAGGACAGGCTG
ACCCAGAGCCTGAACCACCTGCTGACCGCCCTGGACACCACCGACGCCGA
CGTGGCCATCTACTGCAGGGACAAGAAGTGGGAGATGACCCTGAAGGAGG
CCGTGGCCAGGCGGGAGGCCGTGGAGGAGATCTGCATCAGCGACGACAGC
AGCGTGACGGAGCCCGACGCCGAGCTGGTGAGGGTGCACCCCAAGAGCAG
CCTGGCCGGCAGGAAGGGCTACAGCACCAGCGACGGCAAGACCTTCAGCT
ACCTGGAGGGCACCAAGTTCCACCAGGCCGCCAAGGACATCGCCGAGATC
AACGCCATGTGGCCCGTGGCCACCGAGGCCAACGAGCAGGTGTGCATGTA
TATCCTGGGCGAGAGCATGAGCAGCATCAGGAGCAAGTGCCCCGTGGAGG
AGAGCGAGGCCAGCACCCCTCCCAGCACCCTGCCCTGCCTGTGCATCCAC
GCCATGACCCCTGAGAGGGTGCAGCGGCTGAAGGCCAGCAGGCCCGAGCA
GATCACCGTGTGCAGCAGCTTCCCTCTGCCCAAGTACCGGATCACCGGCG
TGCAGAAGATCCAGTGCAGCCAGCCCATCCTGTTCAGCCCCAAGGTGCCC
GCCTACATCCACCCCAGGAAGTACCTGGTGGAGACACCCCCCGTGGACGA
GACACCCGAGCCCAGCGCCGAGAACCAGAGCACCGAGGGCACCCCTGAGC
AGCCTCCCCTGATCACCGAGGACGAGACAAGGACCAGGACGCCCGAGCCC
ATCATCATTGAGGAGGAAGAGGAGGACAGCATCAGCCTGCTGAGCGACGG
CCCCACCCACCAGGTGCTGCAGGTGGAGGCCGACATCCACGGCCCTCCCA
GCGTGAGCAGCTCCAGCTGGAGCATCCCTCACGCCAGCGACTTCGACGTG
GACAGCCTGAGCATCCTGGACACCCTGGAGGGCGCCAGCGTGACCAGCGG
CGCCACCAGCGCCGAGACAAACAGCTACTTCGCCAAGAGCATGGAGTTCC
TGGCCAGGCCCGTGCCCGCCCCTAGGACCGTGTTCAGGAACCCTCCCCAC
CCCGCCCCTAGGACCAGGACCCCTAGCCTGGCCCCTAGCAGGGCCTGCAG
CAGGACCAGCCTGGTGAGCACCCCTCCCGGCGTGAACCGGGTGATCACCA
GGGAGGAGCTGGAGGCCCTGACCCCTAGCAGGACCCCTAGCAGGAGCGTG
AGCAGGACCAGCCTGGTGAGCAACCCTCCCGGCGTGAACCGGGTGATCAC
CAGGGAGGAGTTCGAGGCCTTCGTGGCCCAGCAGCAAAGGCGGTTCGACG
CCGGCGCCTACATCTTCAGCAGCGACACCGGCCAGGGCCACCTGCAGCAG
AAGTCCGTGAGGCAGACCGTGCTGAGCGAGGTGGTCCTGGAGAGGACGGA
GCTGGAGATCAGCTACGCCCCTAGGCTGGACCAGGAGAAGGAGGAGCTGC
TGAGGAAGAAGCTGCAGCTGAACCCCACCCCTGCCAACAGGAGCAGGTAC
CAGAGCAGGAAGGTGGAGAACATGAAGGCCATCACCGCCAGGCGGATCCT
GCAGGGCCTGGGCCACTACCTGAAGGCCGAGGGCAAGGTGGAGTGCTACA
GGACCCTGCACCCCGTGCCCCTGTACTCCAGCTCCGTGAACAGGGCCTTC
AGCAGCCCCAAGGTGGCCGTGGAGGCCTGCAACGCCATGCTGAAGGAGAA
CTTCCCCACCGTGGCCAGCTACTGCATCATCCCCGAGTACGACGCCTACC
TGGACATGGTGGACGGCGCCAGCTGCTGCCTGGACACCGCCAGCTTCTGC
CCCGCCAAGCTGAGGAGCTTCCCCAAGAAGCACAGCTACCTGGAGCCCAC
CATCAGGAGCGCCGTGCCCAGCGCCATCCAGAACACCCTGCAGAACGTGC
TGGCCGCCGCTACCAAGAGGAACTGCAACGTGACCCAGATGAGGGAGCTG
CCCGTGCTGGACAGCGCCGCCTTCAACGTGGAGTGCTTCAAGAAGTACGC
CTGCAACAACGAGTACTGGGAGACATTCAAGGAGAACCCCATCAGGCTGA
CCGAGGAGAACGTGGTGAACTACATCACCAAGCTGAAGGGCCCCAAGGCC
GCCGCTCTGTTCGCCAAGACCCACAACCTGAACATGCTCCAGGACATCCC
TATGGACAGGTTCGTGATGGACCTGAAGAGGGACGTGAAGGTGACCCCTG
GCACCAAGCACACCGAGGAGAGGCCCAAGGTGCAGGTGATCCAGGCCGCC
GACCCTCTGGCCACCGCCTACCTGTGCGGCATCCACAGGGAGCTGGTGAG
GCGGCTGAACGCCGTCCTGCTGCCCAACATCCACACCCTGTTCGACATGA
GCGCCGAGGACTTCGACGCCATCATCGCCGAGCACTTCCAGCCCGGCGAC
TGCGTGCTGGAGACAGACATCGCCAGCTTCGACAAGAGCGAGGACGACGC
TATGGCCCTGACCGCCCTGATGATCCTGGAGGACCTGGGCGTGGACGCCG
AGCTGCTGACCCTGATCGAGGCCGCCTTCGGCGAGATCAGCAGCATCCAC
CTGCCCACCAAGACCAAGTTCAAGTTCGGCGCCATGATGAAGTCCGGCAT
GTTCCTGACCCTGTTCGTGAACACCGTGATCAACATCGTGATCGCCAGCA
GGGTGCTGCGGGAGAGGCTGACCGGCAGCCCCTGCGCCGCCTTCATCGGC
GACGACAACATCGTGAAGGGCGTGAAGTCCGACAAGCTGATGGCCGACAG
GTGCGCCACCTGGCTGAACATGGAGGTGAAGATCATCGACGCCGTGGTGG
GCGAGAAGGCCCCTTACTTCTGCGGCGGCTTCATCCTGTGCGACAGCGTG
ACCGGCACCGCCTGCAGGGTGGCCGACCCTCTGAAGAGGCTGTTCAAGCT
GGGCAAGCCCCTGGCCGCCGACGACGAGCACGACGACGATAGGCGGAGGG
CCCTGCACGAGGAGAGCACCAGGTGGAACCGGGTGGGCATCCTGAGCGAG
CTGTGCAAGGCCGTGGAGAGCAGGTACGAGACAGTGGGCACCAGCATCAT
CGTGATGGCCATGACCACCCTGGCCAGCAGCGTCAAGTCCTTCAGCTACC
TGAGGGGGGCCCCTATAACTCTCTACGGCTAA

SEQ ID NO:7 - Intergenic region (of SEQ ID NOs:1-4)

CCTGAATGGACTACGACATAGTCTAGTCCGCCAAGGCCGCCACC

SEQ ID NO:8 - 3′ UTR (of SEQ ID NOs: 1-4), with poly-A

ACTCGAGTATGTTACGTGCAAAGGTGATTGTCACCCCCCGAAAGACCATA
TTGTGACACACCCTCAGTATCACGCCCAAACATTTACAGCCGCGGTGTCA
AAAACCGCGTGGACGTGGTTAACATCCCTGCTGGGAGGATCAGCCGTAAT
TATTATAATTGGCTTGGTGCTGGCTACTATTGTGGCCATGTACGTGCTGA
CCAACCAGAAACATAATTGAATACAGCAGCAATTGGCAAGCTGCTTACAT
AGAACTCGCGGCGATTGGCATGCCGCCTTAAAATTTTTATTTTATTTTTT
CTTTTCTTTTCCGAATCGGATTTTGTTTTTAATATTTCAAAAAAAAAAAA
AAAAAAAAAAAAATCTAGAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAA

SEQ ID NO:9 - 3′ UTR (of SEQ ID NOs:1-4), without poly-A

ACTCGAGTATGTTACGTGCAAAGGTGATTGTCACCCCCCGAAAGACCATA
TTGTGACACACCCTCAGTATCACGCCCAAACATTTACAGCCGCGGTGTCA
AAAACCGCGTGGACGTGGTTAACATCCCTGCTGGGAGGATCAGCCGTAAT
TATTATAATTGGCTTGGTGCTGGCTACTATTGTGGCCATGTACGTGCTGA
CCAACCAGAAACATAATTGAATACAGCAGCAATTGGCAAGCTGCTTACAT
AGAACTCGCGGCGATTGGCATGCCGCCTTAAAATTTTTATTTTATTTTTT
CTTTTCTTTTCCGAATCGGATTTTGTTTTTAATATTTC

SEQ ID NO:10 - Transgene (nucleic acid sequence; mARM3325/SEQ ID NO:1)

ATGTTCGTGTTCCTGGTGCTGCTGCCCCTGGTGTCTAGCCAGTGCGTGAA
CCTGACCACCAGGACCCAGCTGCCTCCCGCCTACACCAACAGCTTCACCA
GGGGCGTGTACTACCCCGACAAGGTGTTCAGGAGCAGCGTGCTGCACAGC
ACCCAGGACCTGTTCCTGCCCTTCTTCAGCAACGTGACCTGGTTCCACGC
CATCCACGTGAGCGGCACCAACGGCACCAAGAGGTTCGcCAACCCCGTGC
TGCCCTTCAACGACGGCGTGTACTTCGCCAGCACCGAGAAGTCCAACATC
ATCAGGGGCTGGATCTTCGGCACCACCCTGGACAGCAAGACCCAGAGCCT
GCTGATCGTGAACAACGCCACCAACGTGGTGATCAAGGTGTGCGAGTTCC
AGTTCTGCAACGACCCCTTCCTGGGCGTGTACTACCACAAGAACAACAAG
AGCTGGATGGAGAGCGAGTTCAGGGTGTACTCCAGCGCCAACAACTGCAC
CTTCGAGTACGTGAGCCAGCCCTTCCTGATGGACCTGGAGGGCAAGCAGG
GCAACTTCAAGAACCTGAGGGAGTTCGTGTTCAAGAACATCGACGGCTAC
TTCAAGATCTACAGCAAGCACACCCCTATCAACCTGGTGAGGGgCCTGCC
CCAGGGCTTCAGCGCCCTGGAGCCCCTGGTGGACCTGCCCATCGGCATCA
ACATCACCAGGTTCCAGACCCTGCTGGCCCTGCACAGGAGCTACCTGACC
CCTGGCGACAGCAGCTCCGGCTGGACCGCCGGCGCCGCCGCTTACTACGT
GGGCTACCTGCAGCCCAGGACCTTCCTGCTGAAGTACAACGAGAACGGCA
CCATCACCGACGCCGTGGACTGCGCCCTGGACCCTCTGAGCGAGACAAAG
TGCACCCTGAAGTCCTTCACCGTGGAGAAGGGCATCTACCAGACCAGCAA
CTTCAGGGTGCAGCCCACCGAGAGCATCGTGAGGTTCCCCAACATCACCA
ACCTGTGCCCCTTCGGCGAGGTGTTCAACGCCACCAGGTTCGCCAGCGTG
TACGCCTGGAACAGGAAGAGGATCAGCAACTGCGTGGCCGACTACAGCGT
GCTGTATAACAGCGCCAGCTTCAGCACCTTCAAGTGCTACGGCGTGAGCC
CCACCAAGCTGAACGACCTGTGCTTCACCAACGTGTACGCCGACAGCTTC
GTGATCAGGGGCGACGAGGTGAGGCAGATCGCCCCTGGCCAGACCGGCAA
cATCGCCGACTACAACTACAAGCTGCCCGACGACTTCACCGGCTGCGTGA
TCGCCTGGAACAGCAACAACCTGGACAGCAAGGTGGGCGGCAACTACAAC
TACCTGTACCGGCTGTTCAGAAAGAGCAACCTGAAGCCCTTCGAGAGGGA
CATCAGCACCGAGATCTACCAGGCCGGCAGCACCCCTTGCAACGGCGTGa
AGGGCTTCAACTGCTACTTCCCTCTGCAGAGCTACGGCTTCCAGCCCACC
tACGGCGTGGGCTACCAGCCCTACAGGGTGGTGGTCCTGAGCTTCGAGCT
GCTGCACGCCCCTGCCACCGTGTGCGGCCCCAAGAAGTCCACCAACCTGG
TGAAGAACAAGTGCGTGAACTTCAACTTCAACGGCCTGACCGGCACCGGC
GTGCTGACCGAGAGCAACAAGAAGTTCCTGCCCTTCCAGCAGTTCGGCAG
GGACATCGCCGACACCACCGACGCCGTGAGGGACCCTCAGACCCTGGAGA
TCCTGGACATCACCCCTTGCAGCTTCGGCGGCGTGAGCGTGATCACCCCT
GGCACCAACACCAGCAACCAGGTGGCCGTGCTGTACCAGggcGTGAACTG
CACCGAGGTGCCCGTGGCCATCCACGCCGACCAGCTGACCCCTACCTGGA
GGGTGTACTCCACCGGCAGCAACGTGTTCCAGACCAGGGCCGGCTGCCTG
ATCGGCGCCGAGCACGTGAACAACAGCTACGAGTGCGACATCCCCATCGG
CGCCGGCATCTGCGCCAGCTACCAGACCCAGACCAACAGCCCCgGGaGcG
CCAGcAGCGTGGCCAGCCAGAGCATCATCGCCTACACCATGAGCCTGGGC
GtgGAGAACAGCGTGGCCTACAGCAACAACAGCATCGCCATCCCCACCAA
CTTCACCATCAGCGTGACCACCGAGATCCTGCCCGTGAGCATGACCAAGA
CCAGCGTGGACTGCACCATGTATATCTGCGGCGACAGCACCGAGTGCAGC
AACCTGCTGCTCCAGTACGGCAGCTTCTGCACCCAGCTGAACAGGGCCCT
GACCGGCATCGCCGTGGAGCAGGACAAGAACACCCAGGAGGTGTTCGCCC
AGGTGAAGCAGATCTACAAGACCCCTCCCATCAAGGACTTCGGCGGCTTC
AACTTCAGCCAGATCCTGCCCGACCCCAGCAAGCCCAGCAAGAGGAGCTT
CATCGAGGACCTGCTGTTCAACAAGGTGACCCTGGCCGACGCCGGCTTCA
TCAAGCAGTACGGCGACTGCCTGGGCGACATCGCCGCCAGGGACCTGATC
TGCGCCCAGAAGTTCAACGGCCTGACCGTGCTGCCTCCCCTGCTGACCGA
CGAGATGATCGCCCAGTACACCAGCGCCCTGCTGGCCGGCACCATCACCA
GCGGCTGGACCTTCGGCGCCGGCGCCGCCCTGCAGATCCCCTTCGCCATG
CAGATGGCCTACAGGTTCAACGGCATCGGCGTGACCCAGAACGTGCTGTA
CGAGAACCAGAAGCTGATCGCCAACCAGTTCAACAGCGCCATCGGCAAGA
TCCAGGACAGCCTGAGCAGCACCGCCAGCGCCCTGGGCAAGCTGCAGGAC
GTGGTGAACCAGAACGCCCAGGCCCTGAACACCCTGGTGAAGCAGCTGAG
CAGCAACTTCGGCGCCATCAGCAGCGTGCTGAACGACATCCTGAGCAGGC
TGGACccacccGAGGCCGAGGTGCAGATCGACAGGCTGATCACCGGCAGG
CTGCAGAGCCTGCAGACCTACGTGACCCAGCAGCTGATCAGGGCCGCCGA
GATCAGGGCCAGCGCCAACCTGGCCGCCACCAAGATGAGCGAGTGCGTGC
TGGGCCAGAGCAAGAGGGTGGACTTCTGCGGCAAGGGCTACCACCTGATG
AGCTTCCCTCAGAGCGCCCCTCACGGCGTGGTGTTCCTGCACGTGACCTA
CGTGCCCGCCCAGGAGAAGAACTTCACCACAGCCCCTGCCATCTGCCACG
ACGGCAAGGCCCACTTCCCCAGGGAGGGCGTGTTCGTGAGCAACGGCACC
CACTGGTTCGTGACCCAGAGGAACTTCTACGAGCCCCAGATCATCACCAC
CGACAACACCTTCGTGAGCGGCAACTGCGACGTGGTGATCGGCATCGTGA
ACAACACCGTGTACGACCCTCTGCAGCCCGAGCTGGACAGCTTCAAGGAG
GAGCTGGACAAGTACTTCAAGAACCACACCAGCCCCGACGTGGACCTGGG
CGACATCAGCGGCATCAACGCCAGCGTGGTGAACATCCAGAAGGAGATCG
ACAGGCTGAACGAGGTGGCCAAGAACCTGAACGAGAGCCTGATCGACCTG
CAGGAGCTGGGCAAGTACGAGCAGTACATCAAGTGGCCCTGGTACATCTG
GCTGGGCTTCATCGCCGGCCTGATCGCCATCGTGATGGTGACCATCATGC
TGTGCTGCATGACCAGCTGCTGCAGCTGCCTGAAGGGCTGCTGCAGCTGC
GGCAGCTGCTGCAAGTTCGACGAGGACGACAGCGAGCCCGTGCTGAAGGG
CGTGAAGCTGCACTACACCTaA

SEQ ID NO:11 - Transgene (nucleic acid sequence; mARM3280/SEQ ID NO:2)

ATGTTCGTGTTCCTGGTGCTGCTGCCCCTGGTGTCTAGCCAGTGCGTGAA
CCTGACCACCAGGACCCAGCTGCCTCCCGCCTACACCAACAGCTTCACCA
GGGGCGTGTACTACCCCGACAAGGTGTTCAGGAGCAGCGTGCTGCACAGC
ACCCAGGACCTGTTCCTGCCCTTCTTCAGCAACGTGACCTGGTTCCACGC
CATCCACGTGAGCGGCACCAACGGCACCAAGAGGTTCGACAACCCCGTGC
TGCCCTTCAACGACGGCGTGTACTTCGCCAGCACCGAGAAGTCCAACATC
ATCAGGGGCTGGATCTTCGGCACCACCCTGGACAGCAAGACCCAGAGCCT
GCTGATCGTGAACAACGCCACCAACGTGGTGATCAAGGTGTGCGAGTTCC
AGTTCTGCAACGACCCCTTCCTGGGCGTGTACTACCACAAGAACAACAAG
AGCTGGATGGAGAGCGAGTTCAGGGTGTACTCCAGCGCCAACAACTGCAC
CTTCGAGTACGTGAGCCAGCCCTTCCTGATGGACCTGGAGGGCAAGCAGG
GCAACTTCAAGAACCTGAGGGAGTTCGTGTTCAAGAACATCGACGGCTAC
TTCAAGATCTACAGCAAGCACACCCCTATCAACCTGGTGAGGGACCTGCC
CCAGGGCTTCAGCGCCCTGGAGCCCCTGGTGGACCTGCCCATCGGCATCA
ACATCACCAGGTTCCAGACCCTGCTGGCCCTGCACAGGAGCTACCTGACC
CCTGGCGACAGCAGCTCCGGCTGGACCGCCGGCGCCGCCGCTTACTACGT
GGGCTACCTGCAGCCCAGGACCTTCCTGCTGAAGTACAACGAGAACGGCA
CCATCACCGACGCCGTGGACTGCGCCCTGGACCCTCTGAGCGAGACAAAG
TGCACCCTGAAGTCCTTCACCGTGGAGAAGGGCATCTACCAGACCAGCAA
CTTCAGGGTGCAGCCCACCGAGAGCATCGTGAGGTTCCCCAACATCACCA
ACCTGTGCCCCTTCGGCGAGGTGTTCAACGCCACCAGGTTCGCCAGCGTG
TACGCCTGGAACAGGAAGAGGATCAGCAACTGCGTGGCCGACTACAGCGT
GCTGTATAACAGCGCCAGCTTCAGCACCTTCAAGTGCTACGGCGTGAGCC
CCACCAAGCTGAACGACCTGTGCTTCACCAACGTGTACGCCGACAGCTTC
GTGATCAGGGGCGACGAGGTGAGGCAGATCGCCCCTGGCCAGACCGGCAA
GATCGCCGACTACAACTACAAGCTGCCCGACGACTTCACCGGCTGCGTGA
TCGCCTGGAACAGCAACAACCTGGACAGCAAGGTGGGCGGCAACTACAAC
TACCTGTACCGGCTGTTCAGAAAGAGCAACCTGAAGCCCTTCGAGAGGGA
CATCAGCACCGAGATCTACCAGGCCGGCAGCACCCCTTGCAACGGCGTGG
AGGGCTTCAACTGCTACTTCCCTCTGCAGAGCTACGGCTTCCAGCCCACC
AACGGCGTGGGCTACCAGCCCTACAGGGTGGTGGTCCTGAGCTTCGAGCT
GCTGCACGCCCCTGCCACCGTGTGCGGCCCCAAGAAGTCCACCAACCTGG
TGAAGAACAAGTGCGTGAACTTCAACTTCAACGGCCTGACCGGCACCGGC
GTGCTGACCGAGAGCAACAAGAAGTTCCTGCCCTTCCAGCAGTTCGGCAG
GGACATCGCCGACACCACCGACGCCGTGAGGGACCCTCAGACCCTGGAGA
TCCTGGACATCACCCCTTGCAGCTTCGGCGGCGTGAGCGTGATCACCCCT
GGCACCAACACCAGCAACCAGGTGGCCGTGCTGTACCAGggcGTGAACTG
CACCGAGGTGCCCGTGGCCATCCACGCCGACCAGCTGACCCCTACCTGGA
GGGTGTACTCCACCGGCAGCAACGTGTTCCAGACCAGGGCCGGCTGCCTG
ATCGGCGCCGAGCACGTGAACAACAGCTACGAGTGCGACATCCCCATCGG
CGCCGGCATCTGCGCCAGCTACCAGACCCAGACCAACAGCCCCgGGaGcG
CCAGcAGCGTGGCCAGCCAGAGCATCATCGCCTACACCATGAGCCTGGGC
GCCGAGAACAGCGTGGCCTACAGCAACAACAGCATCGCCATCCCCACCAA
CTTCACCATCAGCGTGACCACCGAGATCCTGCCCGTGAGCATGACCAAGA
CCAGCGTGGACTGCACCATGTATATCTGCGGCGACAGCACCGAGTGCAGC
AACCTGCTGCTCCAGTACGGCAGCTTCTGCACCCAGCTGAACAGGGCCCT
GACCGGCATCGCCGTGGAGCAGGACAAGAACACCCAGGAGGTGTTCGCCC
AGGTGAAGCAGATCTACAAGACCCCTCCCATCAAGGACTTCGGCGGCTTC
AACTTCAGCCAGATCCTGCCCGACCCCAGCAAGCCCAGCAAGAGGAGCTT
CATCGAGGACCTGCTGTTCAACAAGGTGACCCTGGCCGACGCCGGCTTCA
TCAAGCAGTACGGCGACTGCCTGGGCGACATCGCCGCCAGGGACCTGATC
TGCGCCCAGAAGTTCAACGGCCTGACCGTGCTGCCTCCCCTGCTGACCGA
CGAGATGATCGCCCAGTACACCAGCGCCCTGCTGGCCGGCACCATCACCA
GCGGCTGGACCTTCGGCGCCGGCGCCGCCCTGCAGATCCCCTTCGCCATG
CAGATGGCCTACAGGTTCAACGGCATCGGCGTGACCCAGAACGTGCTGTA
CGAGAACCAGAAGCTGATCGCCAACCAGTTCAACAGCGCCATCGGCAAGA
TCCAGGACAGCCTGAGCAGCACCGCCAGCGCCCTGGGCAAGCTGCAGGAC
GTGGTGAACCAGAACGCCCAGGCCCTGAACACCCTGGTGAAGCAGCTGAG
CAGCAACTTCGGCGCCATCAGCAGCGTGCTGAACGACATCCTGAGCAGGC
TGGACccacccGAGGCCGAGGTGCAGATCGACAGGCTGATCACCGGCAGG
CTGCAGAGCCTGCAGACCTACGTGACCCAGCAGCTGATCAGGGCCGCCGA
GATCAGGGCCAGCGCCAACCTGGCCGCCACCAAGATGAGCGAGTGCGTGC
TGGGCCAGAGCAAGAGGGTGGACTTCTGCGGCAAGGGCTACCACCTGATG
AGCTTCCCTCAGAGCGCCCCTCACGGCGTGGTGTTCCTGCACGTGACCTA
CGTGCCCGCCCAGGAGAAGAACTTCACCACAGCCCCTGCCATCTGCCACG
ACGGCAAGGCCCACTTCCCCAGGGAGGGCGTGTTCGTGAGCAACGGCACC
CACTGGTTCGTGACCCAGAGGAACTTCTACGAGCCCCAGATCATCACCAC
CGACAACACCTTCGTGAGCGGCAACTGCGACGTGGTGATCGGCATCGTGA
ACAACACCGTGTACGACCCTCTGCAGCCCGAGCTGGACAGCTTCAAGGAG
GAGCTGGACAAGTACTTCAAGAACCACACCAGCCCCGACGTGGACCTGGG
CGACATCAGCGGCATCAACGCCAGCGTGGTGAACATCCAGAAGGAGATCG
ACAGGCTGAACGAGGTGGCCAAGAACCTGAACGAGAGCCTGATCGACCTG
CAGGAGCTGGGCAAGTACGAGCAGTACATCAAGTGGCCCTGGTACATCTG
GCTGGGCTTCATCGCCGGCCTGATCGCCATCGTGATGGTGACCATCATGC
TGTGCTGCATGACCAGCTGCTGCAGCTGCCTGAAGGGCTGCTGCAGCTGC
GGCAGCTGCTGCAAGTTCGACGAGGACGACAGCGAGCCCGTGCTGAAGGG
CGTGAAGCTGCACTACACCTaA

SEQ ID NO:12 - Transgene (nucleic acid sequence; mARM3333/SEQ ID NO:3)

ATGTTCGTGTTCCTGGTGCTGCTGCCCCTGGTGTCTAGCCAGTGCGTGAA
CCTGACCACCAGGACCCAGCTGCCTCCCGCCTACACCAACAGCTTCACCA
GGGGCGTGTACTACCCCGACAAGGTGTTCAGGAGCAGCGTGCTGCACAGC
ACCCAGGACCTGTTCCTGCCCTTCTTCAGCAACGTGACCTGGTTCCACGC
CATCAGCGGCACCAACGGCACCAAGAGGTTCGACAACCCCGTGCTGCCCT
TCAACGACGGCGTGTACTTCGCCAGCACCGAGAAGTCCAACATCATCAGG
GGCTGGATCTTCGGCACCACCCTGGACAGCAAGACCCAGAGCCTGCTGAT
CGTGAACAACGCCACCAACGTGGTGATCAAGGTGTGCGAGTTCCAGTTCT
GCAACGACCCCTTCCTGGGCGTGTACCACAAGAACAACAAGAGCTGGATG
GAGAGCGAGTTCAGGGTGTACTCCAGCGCCAACAACTGCACCTTCGAGTA
CGTGAGCCAGCCCTTCCTGATGGACCTGGAGGGCAAGCAGGGCAACTTCA
AGAACCTGAGGGAGTTCGTGTTCAAGAACATCGACGGCTACTTCAAGATC
TACAGCAAGCACACCCCTATCAACCTGGTGAGGGACCTGCCCCAGGGCTT
CAGCGCCCTGGAGCCCCTGGTGGACCTGCCCATCGGCATCAACATCACCA
GGTTCCAGACCCTGCTGGCCCTGCACAGGAGCTACCTGACCCCTGGCGAC
AGCAGCTCCGGCTGGACCGCCGGCGCCGCCGCTTACTACGTGGGCTACCT
GCAGCCCAGGACCTTCCTGCTGAAGTACAACGAGAACGGCACCATCACCG
ACGCCGTGGACTGCGCCCTGGACCCTCTGAGCGAGACAAAGTGCACCCTG
AAGTCCTTCACCGTGGAGAAGGGCATCTACCAGACCAGCAACTTCAGGGT
GCAGCCCACCGAGAGCATCGTGAGGTTCCCCAACATCACCAACCTGTGCC
CCTTCGGCGAGGTGTTCAACGCCACCAGGTTCGCCAGCGTGTACGCCTGG
AACAGGAAGAGGATCAGCAACTGCGTGGCCGACTACAGCGTGCTGTATAA
CAGCGCCAGCTTCAGCACCTTCAAGTGCTACGGCGTGAGCCCCACCAAGC
TGAACGACCTGTGCTTCACCAACGTGTACGCCGACAGCTTCGTGATCAGG
GGCGACGAGGTGAGGCAGATCGCCCCTGGCCAGACCGGCAAGATCGCCGA
CTACAACTACAAGCTGCCCGACGACTTCACCGGCTGCGTGATCGCCTGGA
ACAGCAACAACCTGGACAGCAAGGTGGGCGGCAACTACAACTACCTGTAC
CGGCTGTTCAGAAAGAGCAACCTGAAGCCCTTCGAGAGGGACATCAGCAC
CGAGATCTACCAGGCCGGCAGCACCCCTTGCAACGGCGTGGAGGGCTTCA
ACTGCTACTTCCCTCTGCAGAGCTACGGCTTCCAGCCCACCtACGGCGTG
GGCTACCAGCCCTACAGGGTGGTGGTCCTGAGCTTCGAGCTGCTGCACGC
CCCTGCCACCGTGTGCGGCCCCAAGAAGTCCACCAACCTGGTGAAGAACA
AGTGCGTGAACTTCAACTTCAACGGCCTGACCGGCACCGGCGTGCTGACC
GAGAGCAACAAGAAGTTCCTGCCCTTCCAGCAGTTCGGCAGGGACATCGa
CGACACCACCGACGCCGTGAGGGACCCTCAGACCCTGGAGATCCTGGACA
TCACCCCTTGCAGCTTCGGCGGCGTGAGCGTGATCACCCCTGGCACCAAC
ACCAGCAACCAGGTGGCCGTGCTGTACCAGGgCGTGAACTGCACCGAGGT
GCCCGTGGCCATCCACGCCGACCAGCTGACCCCTACCTGGAGGGTGTACT
CCACCGGCAGCAACGTGTTCCAGACCAGGGCCGGCTGCCTGATCGGCGCC
GAGCACGTGAACAACAGCTACGAGTGCGACATCCCCATCGGCGCCGGCAT
CTGCGCCAGCTACCAGACCCAGACCAACAGCCaCgGGaGcGCCAGcAGCG
TGGCCAGCCAGAGCATCATCGCCTACACCATGAGCCTGGGCGCCGAGAAC
AGCGTGGCCTACAGCAACAACAGCATCGCCATCCCCAtCAACTTCACCAT
CAGCGTGACCACCGAGATCCTGCCCGTGAGCATGACCAAGACCAGCGTGG
ACTGCACCATGTATATCTGCGGCGACAGCACCGAGTGCAGCAACCTGCTG
CTCCAGTACGGCAGCTTCTGCACCCAGCTGAACAGGGCCCTGACCGGCAT
CGCCGTGGAGCAGGACAAGAACACCCAGGAGGTGTTCGCCCAGGTGAAGC
AGATCTACAAGACCCCTCCCATCAAGGACTTCGGCGGCTTCAACTTCAGC
CAGATCCTGCCCGACCCCAGCAAGCCCAGCAAGAGGAGCTTCATCGAGGA
CCTGCTGTTCAACAAGGTGACCCTGGCCGACGCCGGCTTCATCAAGCAGT
ACGGCGACTGCCTGGGCGACATCGCCGCCAGGGACCTGATCTGCGCCCAG
AAGTTCAACGGCCTGACCGTGCTGCCTCCCCTGCTGACCGACGAGATGAT
CGCCCAGTACACCAGCGCCCTGCTGGCCGGCACCATCACCAGCGGCTGGA
CCTTCGGCGCCGGCGCCGCCCTGCAGATCCCCTTCGCCATGCAGATGGCC
TACAGGTTCAACGGCATCGGCGTGACCCAGAACGTGCTGTACGAGAACCA
GAAGCTGATCGCCAACCAGTTCAACAGCGCCATCGGCAAGATCCAGGACA
GCCTGAGCAGCACCGCCAGCGCCCTGGGCAAGCTGCAGGACGTGGTGAAC
CAGAACGCCCAGGCCCTGAACACCCTGGTGAAGCAGCTGAGCAGCAACTT
CGGCGCCATCAGCAGCGTGCTGAACGACATCCTGgcCAGGCTGGACccac
ccGAGGCCGAGGTGCAGATCGACAGGCTGATCACCGGCAGGCTGCAGAGC
CTGCAGACCTACGTGACCCAGCAGCTGATCAGGGCCGCCGAGATCAGGGC
CAGCGCCAACCTGGCCGCCACCAAGATGAGCGAGTGCGTGCTGGGCCAGA
GCAAGAGGGTGGACTTCTGCGGCAAGGGCTACCACCTGATGAGCTTCCCT
CAGAGCGCCCCTCACGGCGTGGTGTTCCTGCACGTGACCTACGTGCCCGC
CCAGGAGAAGAACTTCACCACAGCCCCTGCCATCTGCCACGACGGCAAGG
CCCACTTCCCCAGGGAGGGCGTGTTCGTGAGCAACGGCACCCACTGGTTC
GTGACCCAGAGGAACTTCTACGAGCCCCAGATCATCACCACCcACAACAC
CTTCGTGAGCGGCAACTGCGACGTGGTGATCGGCATCGTGAACAACACCG
TGTACGACCCTCTGCAGCCCGAGCTGGACAGCTTCAAGGAGGAGCTGGAC
AAGTACTTCAAGAACCACACCAGCCCCGACGTGGACCTGGGCGACATCAG
CGGCATCAACGCCAGCGTGGTGAACATCCAGAAGGAGATCGACAGGCTGA
ACGAGGTGGCCAAGAACCTGAACGAGAGCCTGATCGACCTGCAGGAGCTG
GGCAAGTACGAGCAGTACATCAAGTGGCCCTGGTACATCTGGCTGGGCTT
CATCGCCGGCCTGATCGCCATCGTGATGGTGACCATCATGCTGTGCTGCA
TGACCAGCTGCTGCAGCTGCCTGAAGGGCTGCTGCAGCTGCGGCAGCTGC
TGCAAGTTCGACGAGGACGACAGCGAGCCCGTGCTGAAGGGCGTGAAGCT
GCACTACACCTaA

SEQ ID NO:13 - Transgene (nucleic acid sequence; mARM3346/SEQ ID NO:4)

ATGTTCGTGTTCCTGGTGCTGCTGCCCCTGGTGTCTAGCCAGTGCGTGAA
CtTcACCAaCAGGACCCAGCTGCCTagCGCCTACACCAACAGCTTCACCA
GGGGCGTGTACTACCCCGACAAGGTGTTCAGGAGCAGCGTGCTGCACAGC
ACCCAGGACCTGTTCCTGCCCTTCTTCAGCAACGTGACCTGGTTCCACGC
CATCCACGTGAGCGGCACCAACGGCACCAAGAGGTTCGACAACCCCGTGC
TGCCCTTCAACGACGGCGTGTACTTCGCCAGCACCGAGAAGTCCAACATC
ATCAGGGGCTGGATCTTCGGCACCACCCTGGACAGCAAGACCCAGAGCCT
GCTGATCGTGAACAACGCCACCAACGTGGTGATCAAGGTGTGCGAGTTCC
AGTTCTGCAACtACCCCTTCCTGGGCGTGTACTACCACAAGAACAACAAG
AGCTGGATGGAGAGCGAGTTCAGGGTGTACTCCAGCGCCAACAACTGCAC
CTTCGAGTACGTGAGCCAGCCCTTCCTGATGGACCTGGAGGGCAAGCAGG
GCAACTTCAAGAACCTGAGcGAGTTCGTGTTCAAGAACATCGACGGCTAC
TTCAAGATCTACAGCAAGCACACCCCTATCAACCTGGTGAGGGACCTGCC
CCAGGGCTTCAGCGCCCTGGAGCCCCTGGTGGACCTGCCCATCGGCATCA
ACATCACCAGGTTCCAGACCCTGCTGGCCCTGCACAGGAGCTACCTGACC
CCTGGCGACAGCAGCTCCGGCTGGACCGCCGGCGCCGCCGCTTACTACGT
GGGCTACCTGCAGCCCAGGACCTTCCTGCTGAAGTACAACGAGAACGGCA
CCATCACCGACGCCGTGGACTGCGCCCTGGACCCTCTGAGCGAGACAAAG
TGCACCCTGAAGTCCTTCACCGTGGAGAAGGGCATCTACCAGACCAGCAA
CTTCAGGGTGCAGCCCACCGAGAGCATCGTGAGGTTCCCCAACATCACCA
ACCTGTGCCCCTTCGGCGAGGTGTTCAACGCCACCAGGTTCGCCAGCGTG
TACGCCTGGAACAGGAAGAGGATCAGCAACTGCGTGGCCGACTACAGCGT
GCTGTATAACAGCGCCAGCTTCAGCACCTTCAAGTGCTACGGCGTGAGCC
CCACCAAGCTGAACGACCTGTGCTTCACCAACGTGTACGCCGACAGCTTC
GTGATCAGGGGCGACGAGGTGAGGCAGATCGCCCCTGGCCAGACCGGCAc
cATCGCCGACTACAACTACAAGCTGCCCGACGACTTCACCGGCTGCGTGA
TCGCCTGGAACAGCAACAACCTGGACAGCAAGGTGGGCGGCAACTACAAC
TACCTGTACCGGCTGTTCAGAAAGAGCAACCTGAAGCCCTTCGAGAGGGA
CATCAGCACCGAGATCTACCAGGCCGGCAGCACCCCTTGCAACGGCGTGa
AGGGCTTCAACTGCTACTTCCCTCTGCAGAGCTACGGCTTCCAGCCCACC
tACGGCGTGGGCTACCAGCCCTACAGGGTGGTGGTCCTGAGCTTCGAGCT
GCTGCACGCCCCTGCCACCGTGTGCGGCCCCAAGAAGTCCACCAACCTGG
TGAAGAACAAGTGCGTGAACTTCAACTTCAACGGCCTGACCGGCACCGGC
GTGCTGACCGAGAGCAACAAGAAGTTCCTGCCCTTCCAGCAGTTCGGCAG
GGACATCGCCGACACCACCGACGCCGTGAGGGACCCTCAGACCCTGGAGA
TCCTGGACATCACCCCTTGCAGCTTCGGCGGCGTGAGCGTGATCACCCCT
GGCACCAACACCAGCAACCAGGTGGCCGTGCTGTACCAGggcGTGAACTG
CACCGAGGTGCCCGTGGCCATCCACGCCGACCAGCTGACCCCTACCTGGA
GGGTGTACTCCACCGGCAGCAACGTGTTCCAGACCAGGGCCGGCTGCCTG
ATCGGCGCCGAGtACGTGAACAACAGCTACGAGTGCGACATCCCCATCGG
CGCCGGCATCTGCGCCAGCTACCAGACCCAGACCAACAGCCCCgGGaGcG
CCAGcAGCGTGGCCAGCCAGAGCATCATCGCCTACACCATGAGCCTGGGC
GCCGAGAACAGCGTGGCCTACAGCAACAACAGCATCGCCATCCCCACCAA
CTTCACCATCAGCGTGACCACCGAGATCCTGCCCGTGAGCATGACCAAGA
CCAGCGTGGACTGCACCATGTATATCTGCGGCGACAGCACCGAGTGCAGC
AACCTGCTGCTCCAGTACGGCAGCTTCTGCACCCAGCTGAACAGGGCCCT
GACCGGCATCGCCGTGGAGCAGGACAAGAACACCCAGGAGGTGTTCGCCC
AGGTGAAGCAGATCTACAAGACCCCTCCCATCAAGGACTTCGGCGGCTTC
AACTTCAGCCAGATCCTGCCCGACCCCAGCAAGCCCAGCAAGAGGAGCTT
CATCGAGGACCTGCTGTTCAACAAGGTGACCCTGGCCGACGCCGGCTTCA
TCAAGCAGTACGGCGACTGCCTGGGCGACATCGCCGCCAGGGACCTGATC
TGCGCCCAGAAGTTCAACGGCCTGACCGTGCTGCCTCCCCTGCTGACCGA
CGAGATGATCGCCCAGTACACCAGCGCCCTGCTGGCCGGCACCATCACCA
GCGGCTGGACCTTCGGCGCCGGCGCCGCCCTGCAGATCCCCTTCGCCATG
CAGATGGCCTACAGGTTCAACGGCATCGGCGTGACCCAGAACGTGCTGTA
CGAGAACCAGAAGCTGATCGCCAACCAGTTCAACAGCGCCATCGGCAAGA
TCCAGGACAGCCTGAGCAGCACCGCCAGCGCCCTGGGCAAGCTGCAGGAC
GTGGTGAACCAGAACGCCCAGGCCCTGAACACCCTGGTGAAGCAGCTGAG
CAGCAACTTCGGCGCCATCAGCAGCGTGCTGAACGACATCCTGAGCAGGC
TGGACccacccGAGGCCGAGGTGCAGATCGACAGGCTGATCACCGGCAGG
CTGCAGAGCCTGCAGACCTACGTGACCCAGCAGCTGATCAGGGCCGCCGA
GATCAGGGCCAGCGCCAACCTGGCCGCCAtCAAGATGAGCGAGTGCGTGC
TGGGCCAGAGCAAGAGGGTGGACTTCTGCGGCAAGGGCTACCACCTGATG
AGCTTCCCTCAGAGCGCCCCTCACGGCGTGGTGTTCCTGCACGTGACCTA
CGTGCCCGCCCAGGAGAAGAACTTCACCACAGCCCCTGCCATCTGCCACG
ACGGCAAGGCCCACTTCCCCAGGGAGGGCGTGTTCGTGAGCAACGGCACC
CACTGGTTCGTGACCCAGAGGAACTTCTACGAGCCCCAGATCATCACCAC
CGACAACACCTTCGTGAGCGGCAACTGCGACGTGGTGATCGGCATCGTGA
ACAACACCGTGTACGACCCTCTGCAGCCCGAGCTGGACAGCTTCAAGGAG
GAGCTGGACAAGTACTTCAAGAACCACACCAGCCCCGACGTGGACCTGGG
CGACATCAGCGGCATCAACGCCAGCGTGGTGAACATCCAGAAGGAGATCG
ACAGGCTGAACGAGGTGGCCAAGAACCTGAACGAGAGCCTGATCGACCTG
CAGGAGCTGGGCAAGTACGAGCAGTACATCAAGTGGCCCTGGTACATCTG
GCTGGGCTTCATCGCCGGCCTGATCGCCATCGTGATGGTGACCATCATGC
TGTGCTGCATGACCAGCTGCTGCAGCTGCCTGAAGGGCTGCTGCAGCTGC
GGCAGCTGCTGCAAGTTCGACGAGGACGACAGCGAGCCCGTGCTGAAGGG
CGTGAAGCTGCACTACACCTaA

SEQ ID NO:14 - Transgene (amino acid sequence; mARM3325/SEQ ID NO:1)

MFVFLVLLPLVSSQCVNLTTRTQLPPAYTNSFTRGVYYPDKVFRSSVLHS
TQDLFLPFFSNVTWFHAIHVSGTNGTKRFANPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRGLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGNIADYNYKLPDDFTGCVIAWNSNNLDSKVGGNYN
YLYRLFRKSNLKPFERDISTEIYQAGSTPCNGVKGFNCYFPLQSYGFQPT
YGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQGVNCTEVPVAIHADQLTPTWRVYSTGSNVFQTRAGCL
IGAEHVNNSYECDIPIGAGICASYQTQTNSPGSASSVASQSIIAYTMSLG
VENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT*

SEQ ID NO:15 - Transgene (amino acid sequence; mARM3280/SEQ ID NO:2)

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQGVNCTEVPVAIHADQLTPTWRVYSTGSNVFQTRAGCL
IGAEHVNNSYECDIPIGAGICASYQTQTNSPGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT*

SEQ ID NO:16 - Transgene amino acid sequence; mARM3333/SEQ ID NO:3)

MFVFLVLLPLVSSQCVNLTTRTQLPPAYTNSFTRGVYYPDKVFRSSVLHS
TQDLFLPFFSNVTWFHAISGTNGTKRFDNPVLPFNDGVYFASTEKSNIIR
GWIFGTTLDSKTQSLLIVNNATNVVIKVCEFQFCNDPFLGVYHKNNKSWM
ESEFRVYSSANNCTFEYVSQPFLMDLEGKQGNFKNLREFVFKNIDGYFKI
YSKHTPINLVRDLPQGFSALEPLVDLPIGINITRFQTLLALHRSYLTPGD
SSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAW
NRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLY
RLFRKSNLKPFERDISTEIYQAGSTPCNGVEGFNCYFPLQSYGFQPTYGV
GYQPYRVVVLSFELLHAPATVCGPKKSTNLVKNKCVNFNFNGLTGTGVLT
ESNKKFLPFQQFGRDIDDTTDAVRDPQTLEILDITPCSFGGVSVITPGTN
TSNQVAVLYQGVNCTEVPVAIHADQLTPTWRVYSTGSNVFQTRAGCLIGA
EHVNNSYECDIPIGAGICASYQTQTNSHGSASSVASQSIIAYTMSLGAEN
SVAYSNNSIAIPINFTISVTTEILPVSMTKTSVDCTMYICGDSTECSNLL
LQYGSFCTQLNRALTGIAVEQDKNTQEVFAQVKQIYKTPPIKDFGGFNFS
QILPDPSKPSKRSFIEDLLFNKVTLADAGFIKQYGDCLGDIAARDLICAQ
KFNGLTVLPPLLTDEMIAQYTSALLAGTITSGWTFGAGAALQIPFAMQMA
YRFNGIGVTQNVLYENQKLIANQFNSAIGKIQDSLSSTASALGKLQDVVN
QNAQALNTLVKQLSSNFGAISSVLNDILARLDPPEAEVQIDRLITGRLQS
LQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWF
VTQRNFYEPQIITTHNTFVSGNCDVVIGIVNNTVYDPLQPELDSFKEELD
KYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSC
CKFDEDDSEPVLKGVKLHYT*

SEQ ID NO:17 - Transgene (amino acid sequence; mARM3346/SEQ ID NO:4)

MFVFLVLLPLVSSQCVNFTNRTQLPSAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNYPFLGVYYHKNNK
SWMESEFRVYSSANNCTFEYVSQPFLMDLEGKQGNFKNLSEFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGTIADYNYKLPDDFTGCVIAWNSNNLDSKVGGNYN
YLYRLFRKSNLKPFERDISTEIYQAGSTPCNGVKGFNCYFPLQSYGFQPT
YGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQGVNCTEVPVAIHADQLTPTWRVYSTGSNVFQTRAGCL
IGAEYVNNSYECDIPIGAGICASYQTQTNSPGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAAIKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT*

SEQ ID NO: 18 - mARM3015 (Wuhan; aka ARCT-021)

ATGGGCGGCGCATGAGAGAAGCCCAGACCAATTACCTACCCAAAATGGAG
AAAGTTCACGTTGACATCGAGGAAGACAGCCCATTCCTCAGAGCTTTGCA
GCGGAGCTTCCCGCAGTTTGAGGTAGAAGCCAAGCAGGTCACTGATAATG
ACCATGCTAATGCCAGAGCGTTTTCGCATCTGGCTTCAAAACTGATCGAA
ACGGAGGTGGACCCATCCGACACGATCCTTGACATTGGAAGTGCGCCCGC
CCGCAGAATGTATTCTAAGCACAAGTATCATTGTATCTGTCCGATGAGAT
GTGCGGAAGATCCGGACAGATTGTATAAGTATGCAACTAAGCTGAAGAAA
AACTGTAAGGAAATAACTGATAAGGAATTGGACAAGAAAATGAAGGAGCT
GGCCGCCGTCATGAGCGACCCTGACCTGGAAACTGAGACTATGTGCCTCC
ACGACGACGAGTCGTGTCGCTACGAAGGGCAAGTCGCTGTTTACCAGGAT
GTATACGCCGTCGACGGCCCCACCAGCCTGTACCACCAGGCCAACAAGGG
CGTGAGGGTGGCCTACTGGATCGGCTTCGACACCACACCCTTCATGTTCA
AGAACCTGGCCGGCGCCTACCCCAGCTACAGCACCAACTGGGCCGACGAG
ACCGTGCTGACCGCCAGGAACATCGGCCTGTGCAGCAGCGACGTGATGGA
GAGGAGCCGGAGAGGCATGAGCATCCTGAGGAAGAAATACCTGAAGCCCA
GCAACAACGTGCTGTTCAGCGTGGGCAGCACCATCTACCACGAGAAGAGG
GACCTGCTCAGGAGCTGGCACCTGCCCAGCGTGTTCCACCTGAGGGGCAA
GCAGAACTACACCTGCAGGTGCGAGACCATCGTGAGCTGCGACGGCTACG
TGGTGAAGAGGATCGCCATCAGCCCCGGCCTGTACGGCAAGCCCAGCGGC
TACGCCGCTACAATGCACAGGGAGGGCTTCCTGTGCTGCAAGGTGACCGA
CACCCTGAACGGCGAGAGGGTGAGCTTCCCCGTGTGCACCTACGTGCCCG
CCACCCTGTGCGACCAGATGACCGGCATCCTGGCCACCGACGTGAGCGCC
GACGACGCCCAGAAGCTGCTCGTGGGCCTGAACCAGAGGATCGTGGTCAA
CGGCAGGACCCAGAGGAACACCAACACAATGAAGAACTACCTGCTGCCCG
TGGTGGCCCAGGCTTTCGCCAGGTGGGCCAAGGAGTACAAGGAGGACCAG
GAAGACGAGAGGCCCCTGGGCCTGAGGGACAGGCAGCTGGTGATGGGCTG
CTGCTGGGCCTTCAGGCGGCACAAGATCACCAGCATCTACAAGAGGCCCG
ACACCCAGACCATCATCAAGGTGAACAGCGACTTCCACAGCTTCGTGCTG
CCCAGGATCGGCAGCAACACCCTGGAGATCGGCCTGAGGACCCGGATCAG
GAAGATGCTGGAGGAACACAAGGAGCCCAGCCCACTGATCACCGCCGAGG
ACGTGCAGGAGGCCAAGTGCGCTGCCGACGAGGCCAAGGAGGTGAGGGAG
GCCGAGGAACTGAGGGCCGCCCTGCCACCCCTGGCTGCCGACGTGGAGGA
ACCCACCCTGGAAGCCGACGTGGACCTGATGCTGCAGGAGGCCGGCGCCG
GAAGCGTGGAGACACCCAGGGGCCTGATCAAGGTGACCAGCTACGACGGC
GAGGACAAGATCGGCAGCTACGCCGTGCTGAGCCCACAGGCCGTGCTGAA
GTCCGAGAAGCTGAGCTGCATCCACCCACTGGCCGAGCAGGTGATCGTGA
TCACCCACAGCGGCAGGAAGGGCAGGTACGCCGTGGAGCCCTACCACGGC
AAGGTGGTCGTGCCCGAGGGCCACGCCATCCCCGTGCAGGACTTCCAGGC
CCTGAGCGAGAGCGCCACCATCGTGTACAACGAGAGGGAGTTCGTGAACA
GGTACCTGCACCATATCGCCACCCACGGCGGAGCCCTGAACACCGACGAG
GAATACTACAAGACCGTGAAGCCCAGCGAGCACGACGGCGAGTACCTGTA
CGACATCGACAGGAAGCAGTGCGTGAAGAAAGAGCTGGTGACCGGCCTGG
GACTGACCGGCGAGCTGGTGGACCCACCCTTCCACGAGTTCGCCTACGAG
AGCCTGAGGACCAGACCCGCCGCTCCCTACCAGGTGCCCACCATCGGCGT
GTACGGCGTGCCCGGCAGCGGAAAGAGCGGCATCATCAAGAGCGCCGTGA
CCAAGAAAGACCTGGTGGTCAGCGCCAAGAAAGAGAACTGCGCCGAGATC
ATCAGGGACGTGAAGAAGATGAAAGGCCTGGACGTGAACGCGCGCACCGT
GGACAGCGTGCTGCTGAACGGCTGCAAGCACCCCGTGGAGACCCTGTACA
TCGACGAGGCCTTCGCTTGCCACGCCGGCACCCTGAGGGCCCTGATCGCC
ATCATCAGGCCCAAGAAAGCCGTGCTGTGCGGCGACCCCAAGCAGTGCGG
CTTCTTCAACATGATGTGCCTGAAGGTGCACTTCAACCACGAGATCTGCA
CCCAGGTGTTCCACAAGAGCATCAGCAGGCGGTGCACCAAGAGCGTGACC
AGCGTCGTGAGCACCCTGTTCTACGACAAGAAAATGAGGACCACCAACCC
CAAGGAGACCAAAATCGTGATCGACACCACAGGCAGCACCAAGCCCAAGC
AGGACGACCTGATCCTGACCTGCTTCAGGGGCTGGGTGAAGCAGCTGCAG
ATCGACTACAAGGGCAACGAGATCATGACCGCCGCTGCCAGCCAGGGCCT
GACCAGGAAGGGCGTGTACGCCGTGAGGTACAAGGTGAACGAGAACCCAC
TGTACGCTCCCACCAGCGAGCACGTGAACGTGCTGCTGACCAGGACCGAG
GACAGGATCGTGTGGAAGACCCTGGCCGGCGACCCCTGGATCAAGACCCT
GACCGCCAAGTACCCCGGCAACTTCACCGCCACCATCGAAGAGTGGCAGG
CCGAGCACGACGCCATCATGAGGCACATCCTGGAGAGGCCCGACCCCACC
GACGTGTTCCAGAACAAGGCCAACGTGTGCTGGGCCAAGGCCCTGGTGCC
CGTGCTGAAGACCGCCGGCATCGACATGACCACAGAGCAGTGGAACACCG
TGGACTACTTCGAGACCGACAAGGCCCACAGCGCCGAGATCGTGCTGAAC
CAGCTGTGCGTGAGGTTCTTCGGCCTGGACCTGGACAGCGGCCTGTTCAG
CGCCCCCACCGTGCCACTGAGCATCAGGAACAACCACTGGGACAACAGCC
CCAGCCCAAACATGTACGGCCTGAACAAGGAGGTGGTCAGGCAGCTGAGC
AGGCGGTACCCACAGCTGCCCAGGGCCGTGGCCACCGGCAGGGTGTACGA
CATGAACACCGGCACCCTGAGGAACTACGACCCCAGGATCAACCTGGTGC
CCGTGAACAGGCGGCTGCCCCACGCCCTGGTGCTGCACCACAACGAGCAC
CCACAGAGCGACTTCAGCTCCTTCGTGAGCAAGCTGAAAGGCAGGACCGT
GCTGGTCGTGGGCGAGAAGCTGAGCGTGCCCGGCAAGATGGTGGACTGGC
TGAGCGACAGGCCCGAGGCCACCTTCCGGGCCAGGCTGGACCTCGGCATC
CCCGGCGACGTGCCCAAGTACGACATCATCTTCGTGAACGTCAGGACCCC
ATACAAGTACCACCATTACCAGCAGTGCGAGGACCACGCCATCAAGCTGA
GCATGCTGACCAAGAAGGCCTGCCTGCACCTGAACCCCGGAGGCACCTGC
GTGAGCATCGGCTACGGCTACGCCGACAGGGCCAGCGAGAGCATCATTGG
CGCCATCGCCAGGCTGTTCAAGTTCAGCAGGGTGTGCAAACCCAAGAGCA
GCCTGGAGGAAACCGAGGTGCTGTTCGTGTTCATCGGCTACGACCGGAAG
GCCAGGACCCACAACCCCTACAAGCTGAGCAGCACCCTGACAAACATCTA
CACCGGCAGCAGGCTGCACGAGGCCGGCTGCGCCCCCAGCTACCACGTGG
TCAGGGGCGATATCGCCACCGCCACCGAGGGCGTGATCATCAACGCTGCC
AACAGCAAGGGCCAGCCCGGAGGCGGAGTGTGCGGCGCCCTGTACAAGAA
GTTCCCCGAGAGCTTCGACCTGCAGCCCATCGAGGTGGGCAAGGCCAGGC
TGGTGAAGGGCGCCGCTAAGCACATCATCCACGCCGTGGGCCCCAACTTC
AACAAGGTGAGCGAGGTGGAAGGCGACAAGCAGCTGGCCGAAGCCTACGA
GAGCATCGCCAAGATCGTGAACGACAATAACTACAAGAGCGTGGCCATCC
CACTGCTCAGCACCGGCATCTTCAGCGGCAACAAGGACAGGCTGACCCAG
AGCCTGAACCACCTGCTCACCGCCCTGGACACCACCGATGCCGACGTGGC
CATCTACTGCAGGGACAAGAAGTGGGAGATGACCCTGAAGGAGGCCGTGG
CCAGGCGGGAGGCCGTGGAAGAGATCTGCATCAGCGACGACTCCAGCGTG
ACCGAGCCCGACGCCGAGCTGGTGAGGGTGCACCCCAAGAGCTCCCTGGC
CGGCAGGAAGGGCTACAGCACCAGCGACGGCAAGACCTTCAGCTACCTGG
AGGGCACCAAGTTCCACCAGGCCGCTAAGGACATCGCCGAGATCAACGCT
ATGTGGCCCGTGGCCACCGAGGCCAACGAGCAGGTGTGCATGTACATCCT
GGGCGAGAGCATGTCCAGCATCAGGAGCAAGTGCCCCGTGGAGGAAAGCG
AGGCCAGCACACCACCCAGCACCCTGCCCTGCCTGTGCATCCACGCTATG
ACACCCGAGAGGGTGCAGCGGCTGAAGGCCAGCAGGCCCGAGCAGATCAC
CGTGTGCAGCTCCTTCCCACTGCCCAAGTACAGGATCACCGGCGTGCAGA
AGATCCAGTGCAGCCAGCCCATCCTGTTCAGCCCAAAGGTGCCCGCCTAC
ATCCACCCCAGGAAGTACCTGGTGGAGACCCCACCCGTGGACGAGACACC
CGAGCCAAGCGCCGAGAACCAGAGCACCGAGGGCACACCCGAGCAGCCAC
CCCTGATCACCGAGGACGAGACAAGGACCCGGACCCCAGAGCCCATCATT
ATCGAGGAAGAGGAAGAGGACAGCATCAGCCTGCTGAGCGACGGCCCCAC
CCACCAGGTGCTGCAGGTGGAGGCCGACATCCACGGCCCACCCAGCGTGT
CCAGCTCCAGCTGGAGCATCCCACACGCCAGCGACTTCGACGTGGACAGC
CTGAGCATCCTGGACACCCTGGAGGGCGCCAGCGTGACCTCCGGCGCCAC
CAGCGCCGAGACCAACAGCTACTTCGCCAAGAGCATGGAGTTCCTGGCCA
GGCCCGTGCCAGCTCCCAGGACCGTGTTCAGGAACCCACCCCACCCAGCT
CCCAGGACCAGGACCCCAAGCCTGGCTCCCAGCAGGGCCTGCAGCAGGAC
CAGCCTGGTGAGCACCCCACCCGGCGTGAACAGGGTGATCACCAGGGAGG
AACTGGAGGCCCTGACACCCAGCAGGACCCCCAGCAGGTCCGTGAGCAGG
ACTAGTCTGGTGTCCAACCCACCCGGCGTGAACAGGGTGATCACCAGGGA
GGAATTCGAGGCCTTCGTGGCCCAGCAACAGAGACGGTTCGACGCCGGCG
CCTACATCTTCAGCAGCGACACCGGCCAGGGACACCTGCAGCAAAAGAGC
GTGAGGCAGACCGTGCTGAGCGAGGTGGTGCTGGAGAGGACCGAGCTGGA
AATCAGCTACGCCCCCAGGCTGGACCAGGAGAAGGAGGAACTGCTCAGGA
AGAAACTGCAGCTGAACCCCACCCCAGCCAACAGGAGCAGGTACCAGAGC
AGGAAGGTGGAGAACATGAAGGCCATCACCGCCAGGCGGATCCTGCAGGG
CCTGGGACACTACCTGAAGGCCGAGGGCAAGGTGGAGTGCTACAGGACCC
TGCACCCCGTGCCACTGTACAGCTCCAGCGTGAACAGGGCCTTCTCCAGC
CCCAAGGTGGCCGTGGAGGCCTGCAACGCTATGCTGAAGGAGAACTTCCC
CACCGTGGCCAGCTACTGCATCATCCCCGAGTACGACGCCTACCTGGACA
TGGTGGACGGCGCCAGCTGCTGCCTGGACACCGCCAGCTTCTGCCCCGCC
AAGCTGAGGAGCTTCCCCAAGAAACACAGCTACCTGGAGCCCACCATCAG
GAGCGCCGTGCCCAGCGCCATCCAGAACACCCTGCAGAACGTGCTGGCCG
CTGCCACCAAGAGGAACTGCAACGTGACCCAGATGAGGGAGCTGCCCGTG
CTGGACAGCGCTGCCTTCAACGTGGAGTGCTTCAAGAAATACGCCTGCAA
CAACGAGTACTGGGAGACCTTCAAGGAGAACCCCATCAGGCTGACCGAAG
AGAACGTGGTGAACTACATCACCAAGCTGAAGGGCCCCAAGGCCGCTGCC
CTGTTCGCTAAGACCCACAACCTGAACATGCTGCAGGACATCCCAATGGA
CAGGTTCGTGATGGACCTGAAGAGGGACGTGAAGGTGACACCCGGCACCA
AGCACACCGAGGAGAGGCCCAAGGTGCAGGTGATCCAGGCCGCTGACCCA
CTGGCCACCGCCTACCTGTGCGGCATCCACAGGGAGCTGGTGAGGCGGCT
GAACGCCGTGCTGCTGCCCAACATCCACACCCTGTTCGACATGAGCGCCG
AGGACTTCGACGCCATCATCGCCGAGCACTTCCAGCCCGGCGACTGCGTG
CTGGAGACCGACATCGCCAGCTTCGACAAGAGCGAGGATGACGCTATGGC
CCTGACCGCTCTGATGATCCTGGAGGACCTGGGCGTGGACGCCGAGCTGC
TCACCCTGATCGAGGCTGCCTTCGGCGAGATCAGCTCCATCCACCTGCCC
ACCAAGACCAAGTTCAAGTTCGGCGCTATGATGAAAAGCGGAATGTTCCT
GACCCTGTTCGTGAACACCGTGATCAACATTGTGATCGCCAGCAGGGTGC
TGCGGGAGAGGCTGACCGGCAGCCCCTGCGCTGCCTTCATCGGCGACGAC
AACATCGTGAAGGGCGTGAAAAGCGACAAGCTGATGGCCGACAGGTGCGC
CACCTGGCTGAACATGGAGGTGAAGATCATCGACGCCGTGGTGGGCGAGA
AGGCCCCCTACTTCTGCGGCGGATTCATCCTGTGCGACAGCGTGACCGGC
ACCGCCTGCAGGGTGGCCGACCCCCTGAAGAGGCTGTTCAAGCTGGGCAA
GCCACTGGCCGCTGACGATGAGCACGACGATGACAGGCGGAGGGCCCTGC
ACGAGGAAAGCACCAGGTGGAACAGGGTGGGCATCCTGAGCGAGCTGTGC
AAGGCCGTGGAGAGCAGGTACGAGACCGTGGGCACCAGCATCATCGTGAT
GGCTATGACCACACTGGCCAGCTCCGTCAAGAGCTTCTCCTACCTGAGGG
GGGCCCCTATAACTCTCTACGGCTAACCTGAATGGACTACGACATAGTCT
AGTCCGCCAAGGCCGCCACCATGTTCGTCTTCCTGGTCCTGCTGCCTCTG
GTCTCCTCACAGTGCGTCAATCTGACAACTCGGACTCAGCTGCCACCTGC
TTATACTAATAGCTTCACCAGAGGCGTGTACTATCCTGACAAGGTGTTTA
GAAGCTCCGTGCTGCACTCTACACAGGATCTGTTTCTGCCATTCTTTAGC
AACGTGACCTGGTTCCACGCCATCCACGTGAGCGGCACCAATGGCACAAA
GCGGTTCGACAATCCCGTGCTGCCTTTTAACGATGGCGTGTACTTCGCCT
CTACCGAGAAGTCCAACATCATCAGAGGCTGGATCTTTGGCACCACACTG
GACTCCAAGACACAGTCTCTGCTGATCGTGAACAATGCCACCAACGTGGT
CATCAAGGTGTGCGAGTTCCAGTTTTGTAATGATCCCTTCCTGGGCGTGT
ACTATCACAAGAACAATAAGAGCTGGATGGAGTCCGAGTTTAGAGTGTAT
TCTAGCGCCAACAACTGCACATTTGAGTACGTGAGCCAGCCTTTCCTGAT
GGACCTGGAGGGCAAGCAGGGCAATTTCAAGAACCTGAGGGAGTTCGTGT
TTAAGAATATCGACGGCTACTTCAAAATCTACTCTAAGCACACCCCCATC
AACCTGGTGCGCGACCTGCCTCAGGGCTTCAGCGCCCTGGAGCCCCTGGT
GGATCTGCCTATCGGCATCAACATCACCCGGTTTCAGACACTGCTGGCCC
TGCACAGAAGCTACCTGACACCCGGCGACTCCTCTAGCGGATGGACCGCC
GGCGCTGCCGCCTACTATGTGGGCTACCTCCAGCCCCGGACCTTCCTGCT
GAAGTACAACGAGAATGGCACCATCACAGACGCAGTGGATTGCGCCCTGG
ACCCCCTGAGCGAGACAAAGTGTACACTGAAGTCCTTTACCGTGGAGAAG
GGCATCTATCAGACATCCAATTTCAGGGTGCAGCCAACCGAGTCTATCGT
GCGCTTTCCTAATATCACAAACCTGTGCCCATTTGGCGAGGTGTTCAACG
CAACCCGCTTCGCCAGCGTGTACGCCTGGAATAGGAAGCGGATCAGCAAC
TGCGTGGCCGACTATAGCGTGCTGTACAACTCCGCCTCTTTCAGCACCTT
TAAGTGCTATGGCGTGTCCCCCACAAAGCTGAATGACCTGTGCTTTACCA
ACGTCTACGCCGATTCTTTCGTGATCAGGGGCGACGAGGTGCGCCAGATC
GCCCCCGGCCAGACAGGCAAGATCGCAGACTACAATTATAAGCTGCCAGA
CGATTTCACCGGCTGCGTGATCGCCTGGAACAGCAACAATCTGGATTCCA
AAGTGGGCGGCAACTACAATTATCTGTACCGGCTGTTTAGAAAGAGCAAT
CTGAAGCCCTTCGAGAGGGACATCTCTACAGAAATCTACCAGGCCGGCAG
CACCCCTTGCAATGGCGTGGAGGGCTTTAACTGTTATTTCCCACTCCAGT
CCTACGGCTTCCAGCCCACAAACGGCGTGGGCTATCAGCCTTACCGCGTG
GTGGTGCTGAGCTTTGAGCTGCTGCACGCCCCAGCAACAGTGTGCGGCCC
CAAGAAGTCCACCAATCTGGTGAAGAACAAGTGCGTGAACTTCAACTTCA
ACGGCCTGACCGGCACAGGCGTGCTGACCGAGTCCAACAAGAAGTTCCTG
CCATTTCAGCAGTTCGGCAGGGACATCGCAGATACCACAGACGCCGTGCG
CGACCCACAGACCCTGGAGATCCTGGACATCACACCCTGCTCTTTCGGCG
GCGTGAGCGTGATCACACCCGGCACCAATACAAGCAACCAGGTGGCCGTG
CTGTATCAGGACGTGAATTGTACCGAGGTGCCCGTGGCTATCCACGCCGA
TCAGCTGACCCCAACATGGCGGGTGTACAGCACCGGCTCCAACGTCTTCC
AGACAAGAGCCGGATGCCTGATCGGAGCAGAGCACGTGAACAATTCCTAT
GAGTGCGACATCCCAATCGGCGCCGGCATCTGTGCCTCTTACCAGACCCA
GACAAACTCTCCCAGACGGGCCCGGAGCGTGGCCTCCCAGTCTATCATCG
CCTATACCATGTCCCTGGGCGCCGAGAACAGCGTGGCCTACTCTAACAAT
AGCATCGCCATCCCAACCAACTTCACAATCTCTGTGACCACAGAGATCCT
GCCCGTGTCCATGACCAAGACATCTGTGGACTGCACAATGTATATCTGTG
GCGATTCTACCGAGTGCAGCAACCTGCTGCTCCAGTACGGCAGCTTTTGT
ACCCAGCTGAATAGAGCCCTGACAGGCATCGCCGTGGAGCAGGATAAGAA
CACACAGGAGGTGTTCGCCCAGGTGAAGCAAATCTACAAGACCCCCCCTA
TCAAGGACTTTGGCGGCTTCAATTTTTCCCAGATCCTGCCTGATCCATCC
AAGCCTTCTAAGCGGAGCTTTATCGAGGACCTGCTGTTCAACAAGGTGAC
CCTGGCCGATGCCGGCTTCATCAAGCAGTATGGCGATTGCCTGGGCGACA
TCGCAGCCAGGGACCTGATCTGCGCCCAGAAGTTTAATGGCCTGACCGTG
CTGCCACCCCTGCTGACAGATGAGATGATCGCACAGTACACAAGCGCCCT
GCTGGCCGGCACCATCACATCCGGATGGACCTTCGGCGCAGGAGCCGCCC
TCCAGATCCCCTTTGCCATGCAGATGGCCTATAGGTTCAACGGCATCGGC
GTGACCCAGAATGTGCTGTACGAGAACCAGAAGCTGATCGCCAATCAGTT
TAACTCCGCCATCGGCAAGATCCAGGACAGCCTGTCCTCTACAGCCAGCG
CCCTGGGCAAGCTCCAGGATGTGGTGAATCAGAACGCCCAGGCCCTGAAT
ACCCTGGTGAAGCAGCTGAGCAGCAACTTCGGCGCCATCTCTAGCGTGCT
GAATGACATCCTGAGCCGGCTGGACAAGGTGGAGGCAGAGGTGCAGATCG
ACCGGCTGATCACCGGCCGGCTCCAGAGCCTCCAGACCTATGTGACACAG
CAGCTGATCAGGGCCGCCGAGATCAGGGCCAGCGCCAATCTGGCAGCAAC
CAAGATGTCCGAGTGCGTGCTGGGCCAGTCTAAGAGAGTGGACTTTTGTG
GCAAGGGCTATCACCTGATGTCCTTCCCTCAGTCTGCCCCACACGGCGTG
GTGTTTCTGCACGTGACCTACGTGCCCGCCCAGGAGAAGAACTTCACCAC
AGCCCCTGCCATCTGCCACGATGGCAAGGCCCACTTTCCAAGGGAGGGCG
TGTTCGTGTCCAACGGCACCCACTGGTTTGTGACACAGCGCAATTTCTAC
GAGCCCCAGATCATCACCACAGACAACACCTTCGTGAGCGGCAACTGTGA
CGTGGTCATCGGCATCGTGAACAATACCGTGTATGATCCACTCCAGCCCG
AGCTGGACAGCTTTAAGGAGGAGCTGGATAAGTATTTCAAGAATCACACC
TCCCCTGACGTGGATCTGGGCGACATCAGCGGCATCAATGCCTCCGTGGT
GAACATCCAGAAGGAGATCGACCGCCTGAACGAGGTGGCTAAGAATCTGA
ACGAGAGCCTGATCGACCTCCAGGAGCTGGGCAAGTATGAGCAGTACATC
AAGTGGCCCTGGTACATCTGGCTGGGCTTCATCGCCGGCCTGATCGCCAT
CGTGATGGTGACCATCATGCTGTGCTGTATGACATCCTGCTGTTCTTGCC
TGAAGGGCTGCTGTAGCTGTGGCTCCTGCTGTAAGTTTGACGAGGATGAC
TCTGAACCTGTGCTGAAGGGCGTGAAGCTGCATTACACCTAAACTCGAGT
ATGTTACGTGCAAAGGTGATTGTCACCCCCCGAAAGACCATATTGTGACA
CACCCTCAGTATCACGCCCAAACATTTACAGCCGCGGTGTCAAAAACCGC
GTGGACGTGGTTAACATCCCTGCTGGGAGGATCAGCCGTAATTATTATAA
TTGGCTTGGTGCTGGCTACTATTGTGGCCATGTACGTGCTGACCAACCAG
AAACATAATTGAATACAGCAGCAATTGGCAAGCTGCTTACATAGAACTCG
CGGCGATTGGCATGCCGCCTTAAAATTTTTATTTTATTTTTTCTTTTCTT
TTCCGAATCGGATTTTGTTTTTAATATTTCAAAAAAAAAAAAAAAAAAAA
AAAAATCTAGAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAA

SEQ ID NO:19 - 5′ UTR (of mARM3015/SEQ ID NO:18)

ATGGGCGGCGCATGAGAGAAGCCCAGACCAATTACCTACCCAAA

SEQ ID NO:20 - nsP1-4 (of mARM3015/SEQ ID NO:18)

ATGGAGAAAGTTCACGTTGACATCGAGGAAGACAGCCCATTCCTCAGAGC
TTTGCAGCGGAGCTTCCCGCAGTTTGAGGTAGAAGCCAAGCAGGTCACTG
ATAATGACCATGCTAATGCCAGAGCGTTTTCGCATCTGGCTTCAAAACTG
ATCGAAACGGAGGTGGACCCATCCGACACGATCCTTGACATTGGAAGTGC
GCCCGCCCGCAGAATGTATTCTAAGCACAAGTATCATTGTATCTGTCCGA
TGAGATGTGCGGAAGATCCGGACAGATTGTATAAGTATGCAACTAAGCTG
AAGAAAAACTGTAAGGAAATAACTGATAAGGAATTGGACAAGAAAATGAA
GGAGCTGGCCGCCGTCATGAGCGACCCTGACCTGGAAACTGAGACTATGT
GCCTCCACGACGACGAGTCGTGTCGCTACGAAGGGCAAGTCGCTGTTTAC
CAGGATGTATACGCCGTCGACGGCCCCACCAGCCTGTACCACCAGGCCAA
CAAGGGCGTGAGGGTGGCCTACTGGATCGGCTTCGACACCACACCCTTCA
TGTTCAAGAACCTGGCCGGCGCCTACCCCAGCTACAGCACCAACTGGGCC
GACGAGACCGTGCTGACCGCCAGGAACATCGGCCTGTGCAGCAGCGACGT
GATGGAGAGGAGCCGGAGAGGCATGAGCATCCTGAGGAAGAAATACCTGA
AGCCCAGCAACAACGTGCTGTTCAGCGTGGGCAGCACCATCTACCACGAG
AAGAGGGACCTGCTCAGGAGCTGGCACCTGCCCAGCGTGTTCCACCTGAG
GGGCAAGCAGAACTACACCTGCAGGTGCGAGACCATCGTGAGCTGCGACG
GCTACGTGGTGAAGAGGATCGCCATCAGCCCCGGCCTGTACGGCAAGCCC
AGCGGCTACGCCGCTACAATGCACAGGGAGGGCTTCCTGTGCTGCAAGGT
GACCGACACCCTGAACGGCGAGAGGGTGAGCTTCCCCGTGTGCACCTACG
TGCCCGCCACCCTGTGCGACCAGATGACCGGCATCCTGGCCACCGACGTG
AGCGCCGACGACGCCCAGAAGCTGCTCGTGGGCCTGAACCAGAGGATCGT
GGTCAACGGCAGGACCCAGAGGAACACCAACACAATGAAGAACTACCTGC
TGCCCGTGGTGGCCCAGGCTTTCGCCAGGTGGGCCAAGGAGTACAAGGAG
GACCAGGAAGACGAGAGGCCCCTGGGCCTGAGGGACAGGCAGCTGGTGAT
GGGCTGCTGCTGGGCCTTCAGGCGGCACAAGATCACCAGCATCTACAAGA
GGCCCGACACCCAGACCATCATCAAGGTGAACAGCGACTTCCACAGCTTC
GTGCTGCCCAGGATCGGCAGCAACACCCTGGAGATCGGCCTGAGGACCCG
GATCAGGAAGATGCTGGAGGAACACAAGGAGCCCAGCCCACTGATCACCG
CCGAGGACGTGCAGGAGGCCAAGTGCGCTGCCGACGAGGCCAAGGAGGTG
AGGGAGGCCGAGGAACTGAGGGCCGCCCTGCCACCCCTGGCTGCCGACGT
GGAGGAACCCACCCTGGAAGCCGACGTGGACCTGATGCTGCAGGAGGCCG
GCGCCGGAAGCGTGGAGACACCCAGGGGCCTGATCAAGGTGACCAGCTAC
GACGGCGAGGACAAGATCGGCAGCTACGCCGTGCTGAGCCCACAGGCCGT
GCTGAAGTCCGAGAAGCTGAGCTGCATCCACCCACTGGCCGAGCAGGTGA
TCGTGATCACCCACAGCGGCAGGAAGGGCAGGTACGCCGTGGAGCCCTAC
CACGGCAAGGTGGTCGTGCCCGAGGGCCACGCCATCCCCGTGCAGGACTT
CCAGGCCCTGAGCGAGAGCGCCACCATCGTGTACAACGAGAGGGAGTTCG
TGAACAGGTACCTGCACCATATCGCCACCCACGGCGGAGCCCTGAACACC
GACGAGGAATACTACAAGACCGTGAAGCCCAGCGAGCACGACGGCGAGTA
CCTGTACGACATCGACAGGAAGCAGTGCGTGAAGAAAGAGCTGGTGACCG
GCCTGGGACTGACCGGCGAGCTGGTGGACCCACCCTTCCACGAGTTCGCC
TACGAGAGCCTGAGGACCAGACCCGCCGCTCCCTACCAGGTGCCCACCAT
CGGCGTGTACGGCGTGCCCGGCAGCGGAAAGAGCGGCATCATCAAGAGCG
CCGTGACCAAGAAAGACCTGGTGGTCAGCGCCAAGAAAGAGAACTGCGCC
GAGATCATCAGGGACGTGAAGAAGATGAAAGGCCTGGACGTGAACGCGCG
CACCGTGGACAGCGTGCTGCTGAACGGCTGCAAGCACCCCGTGGAGACCC
TGTACATCGACGAGGCCTTCGCTTGCCACGCCGGCACCCTGAGGGCCCTG
ATCGCCATCATCAGGCCCAAGAAAGCCGTGCTGTGCGGCGACCCCAAGCA
GTGCGGCTTCTTCAACATGATGTGCCTGAAGGTGCACTTCAACCACGAGA
TCTGCACCCAGGTGTTCCACAAGAGCATCAGCAGGCGGTGCACCAAGAGC
GTGACCAGCGTCGTGAGCACCCTGTTCTACGACAAGAAAATGAGGACCAC
CAACCCCAAGGAGACCAAAATCGTGATCGACACCACAGGCAGCACCAAGC
CCAAGCAGGACGACCTGATCCTGACCTGCTTCAGGGGCTGGGTGAAGCAG
CTGCAGATCGACTACAAGGGCAACGAGATCATGACCGCCGCTGCCAGCCA
GGGCCTGACCAGGAAGGGCGTGTACGCCGTGAGGTACAAGGTGAACGAGA
ACCCACTGTACGCTCCCACCAGCGAGCACGTGAACGTGCTGCTGACCAGG
ACCGAGGACAGGATCGTGTGGAAGACCCTGGCCGGCGACCCCTGGATCAA
GACCCTGACCGCCAAGTACCCCGGCAACTTCACCGCCACCATCGAAGAGT
GGCAGGCCGAGCACGACGCCATCATGAGGCACATCCTGGAGAGGCCCGAC
CCCACCGACGTGTTCCAGAACAAGGCCAACGTGTGCTGGGCCAAGGCCCT
GGTGCCCGTGCTGAAGACCGCCGGCATCGACATGACCACAGAGCAGTGGA
ACACCGTGGACTACTTCGAGACCGACAAGGCCCACAGCGCCGAGATCGTG
CTGAACCAGCTGTGCGTGAGGTTCTTCGGCCTGGACCTGGACAGCGGCCT
GTTCAGCGCCCCCACCGTGCCACTGAGCATCAGGAACAACCACTGGGACA
ACAGCCCCAGCCCAAACATGTACGGCCTGAACAAGGAGGTGGTCAGGCAG
CTGAGCAGGCGGTACCCACAGCTGCCCAGGGCCGTGGCCACCGGCAGGGT
GTACGACATGAACACCGGCACCCTGAGGAACTACGACCCCAGGATCAACC
TGGTGCCCGTGAACAGGCGGCTGCCCCACGCCCTGGTGCTGCACCACAAC
GAGCACCCACAGAGCGACTTCAGCTCCTTCGTGAGCAAGCTGAAAGGCAG
GACCGTGCTGGTCGTGGGCGAGAAGCTGAGCGTGCCCGGCAAGATGGTGG
ACTGGCTGAGCGACAGGCCCGAGGCCACCTTCCGGGCCAGGCTGGACCTC
GGCATCCCCGGCGACGTGCCCAAGTACGACATCATCTTCGTGAACGTCAG
GACCCCATACAAGTACCACCATTACCAGCAGTGCGAGGACCACGCCATCA
AGCTGAGCATGCTGACCAAGAAGGCCTGCCTGCACCTGAACCCCGGAGGC
ACCTGCGTGAGCATCGGCTACGGCTACGCCGACAGGGCCAGCGAGAGCAT
CATTGGCGCCATCGCCAGGCTGTTCAAGTTCAGCAGGGTGTGCAAACCCA
AGAGCAGCCTGGAGGAAACCGAGGTGCTGTTCGTGTTCATCGGCTACGAC
CGGAAGGCCAGGACCCACAACCCCTACAAGCTGAGCAGCACCCTGACAAA
CATCTACACCGGCAGCAGGCTGCACGAGGCCGGCTGCGCCCCCAGCTACC
ACGTGGTCAGGGGCGATATCGCCACCGCCACCGAGGGCGTGATCATCAAC
GCTGCCAACAGCAAGGGCCAGCCCGGAGGCGGAGTGTGCGGCGCCCTGTA
CAAGAAGTTCCCCGAGAGCTTCGACCTGCAGCCCATCGAGGTGGGCAAGG
CCAGGCTGGTGAAGGGCGCCGCTAAGCACATCATCCACGCCGTGGGCCCC
AACTTCAACAAGGTGAGCGAGGTGGAAGGCGACAAGCAGCTGGCCGAAGC
CTACGAGAGCATCGCCAAGATCGTGAACGACAATAACTACAAGAGCGTGG
CCATCCCACTGCTCAGCACCGGCATCTTCAGCGGCAACAAGGACAGGCTG
ACCCAGAGCCTGAACCACCTGCTCACCGCCCTGGACACCACCGATGCCGA
CGTGGCCATCTACTGCAGGGACAAGAAGTGGGAGATGACCCTGAAGGAGG
CCGTGGCCAGGCGGGAGGCCGTGGAAGAGATCTGCATCAGCGACGACTCC
AGCGTGACCGAGCCCGACGCCGAGCTGGTGAGGGTGCACCCCAAGAGCTC
CCTGGCCGGCAGGAAGGGCTACAGCACCAGCGACGGCAAGACCTTCAGCT
ACCTGGAGGGCACCAAGTTCCACCAGGCCGCTAAGGACATCGCCGAGATC
AACGCTATGTGGCCCGTGGCCACCGAGGCCAACGAGCAGGTGTGCATGTA
CATCCTGGGCGAGAGCATGTCCAGCATCAGGAGCAAGTGCCCCGTGGAGG
AAAGCGAGGCCAGCACACCACCCAGCACCCTGCCCTGCCTGTGCATCCAC
GCTATGACACCCGAGAGGGTGCAGCGGCTGAAGGCCAGCAGGCCCGAGCA
GATCACCGTGTGCAGCTCCTTCCCACTGCCCAAGTACAGGATCACCGGCG
TGCAGAAGATCCAGTGCAGCCAGCCCATCCTGTTCAGCCCAAAGGTGCCC
GCCTACATCCACCCCAGGAAGTACCTGGTGGAGACCCCACCCGTGGACGA
GACACCCGAGCCAAGCGCCGAGAACCAGAGCACCGAGGGCACACCCGAGC
AGCCACCCCTGATCACCGAGGACGAGACAAGGACCCGGACCCCAGAGCCC
ATCATTATCGAGGAAGAGGAAGAGGACAGCATCAGCCTGCTGAGCGACGG
CCCCACCCACCAGGTGCTGCAGGTGGAGGCCGACATCCACGGCCCACCCA
GCGTGTCCAGCTCCAGCTGGAGCATCCCACACGCCAGCGACTTCGACGTG
GACAGCCTGAGCATCCTGGACACCCTGGAGGGCGCCAGCGTGACCTCCGG
CGCCACCAGCGCCGAGACCAACAGCTACTTCGCCAAGAGCATGGAGTTCC
TGGCCAGGCCCGTGCCAGCTCCCAGGACCGTGTTCAGGAACCCACCCCAC
CCAGCTCCCAGGACCAGGACCCCAAGCCTGGCTCCCAGCAGGGCCTGCAG
CAGGACCAGCCTGGTGAGCACCCCACCCGGCGTGAACAGGGTGATCACCA
GGGAGGAACTGGAGGCCCTGACACCCAGCAGGACCCCCAGCAGGTCCGTG
AGCAGGACTAGTCTGGTGTCCAACCCACCCGGCGTGAACAGGGTGATCAC
CAGGGAGGAATTCGAGGCCTTCGTGGCCCAGCAACAGAGACGGTTCGACG
CCGGCGCCTACATCTTCAGCAGCGACACCGGCCAGGGACACCTGCAGCAA
AAGAGCGTGAGGCAGACCGTGCTGAGCGAGGTGGTGCTGGAGAGGACCGA
GCTGGAAATCAGCTACGCCCCCAGGCTGGACCAGGAGAAGGAGGAACTGC
TCAGGAAGAAACTGCAGCTGAACCCCACCCCAGCCAACAGGAGCAGGTAC
CAGAGCAGGAAGGTGGAGAACATGAAGGCCATCACCGCCAGGCGGATCCT
GCAGGGCCTGGGACACTACCTGAAGGCCGAGGGCAAGGTGGAGTGCTACA
GGACCCTGCACCCCGTGCCACTGTACAGCTCCAGCGTGAACAGGGCCTTC
TCCAGCCCCAAGGTGGCCGTGGAGGCCTGCAACGCTATGCTGAAGGAGAA
CTTCCCCACCGTGGCCAGCTACTGCATCATCCCCGAGTACGACGCCTACC
TGGACATGGTGGACGGCGCCAGCTGCTGCCTGGACACCGCCAGCTTCTGC
CCCGCCAAGCTGAGGAGCTTCCCCAAGAAACACAGCTACCTGGAGCCCAC
CATCAGGAGCGCCGTGCCCAGCGCCATCCAGAACACCCTGCAGAACGTGC
TGGCCGCTGCCACCAAGAGGAACTGCAACGTGACCCAGATGAGGGAGCTG
CCCGTGCTGGACAGCGCTGCCTTCAACGTGGAGTGCTTCAAGAAATACGC
CTGCAACAACGAGTACTGGGAGACCTTCAAGGAGAACCCCATCAGGCTGA
CCGAAGAGAACGTGGTGAACTACATCACCAAGCTGAAGGGCCCCAAGGCC
GCTGCCCTGTTCGCTAAGACCCACAACCTGAACATGCTGCAGGACATCCC
AATGGACAGGTTCGTGATGGACCTGAAGAGGGACGTGAAGGTGACACCCG
GCACCAAGCACACCGAGGAGAGGCCCAAGGTGCAGGTGATCCAGGCCGCT
GACCCACTGGCCACCGCCTACCTGTGCGGCATCCACAGGGAGCTGGTGAG
GCGGCTGAACGCCGTGCTGCTGCCCAACATCCACACCCTGTTCGACATGA
GCGCCGAGGACTTCGACGCCATCATCGCCGAGCACTTCCAGCCCGGCGAC
TGCGTGCTGGAGACCGACATCGCCAGCTTCGACAAGAGCGAGGATGACGC
TATGGCCCTGACCGCTCTGATGATCCTGGAGGACCTGGGCGTGGACGCCG
AGCTGCTCACCCTGATCGAGGCTGCCTTCGGCGAGATCAGCTCCATCCAC
CTGCCCACCAAGACCAAGTTCAAGTTCGGCGCTATGATGAAAAGCGGAAT
GTTCCTGACCCTGTTCGTGAACACCGTGATCAACATTGTGATCGCCAGCA
GGGTGCTGCGGGAGAGGCTGACCGGCAGCCCCTGCGCTGCCTTCATCGGC
GACGACAACATCGTGAAGGGCGTGAAAAGCGACAAGCTGATGGCCGACAG
GTGCGCCACCTGGCTGAACATGGAGGTGAAGATCATCGACGCCGTGGTGG
GCGAGAAGGCCCCCTACTTCTGCGGCGGATTCATCCTGTGCGACAGCGTG
ACCGGCACCGCCTGCAGGGTGGCCGACCCCCTGAAGAGGCTGTTCAAGCT
GGGCAAGCCACTGGCCGCTGACGATGAGCACGACGATGACAGGCGGAGGG
CCCTGCACGAGGAAAGCACCAGGTGGAACAGGGTGGGCATCCTGAGCGAG
CTGTGCAAGGCCGTGGAGAGCAGGTACGAGACCGTGGGCACCAGCATCAT
CGTGATGGCTATGACCACACTGGCCAGCTCCGTCAAGAGCTTCTCCTACC
TGAGGGGGGCCCCTATAACTCTCTACGGCTAA

SEQ ID NO:21 - Intergenic region (of SEQ ID NO:18)

CCTGAATGGACTACGACATAGTCTAGTCCGCCAAGGCCGCCACC

SEQ ID NO:22 - 3′ UTR (of SEQ NO:18), with poly-A

ACTCGAGTATGTTACGTGCAAAGGTGATTGTCACCCCCCGAAAGACCATA
TTGTGACACACCCTCAGTATCACGCCCAAACATTTACAGCCGCGGTGTCA
AAAACCGCGTGGACGTGGTTAACATCCCTGCTGGGAGGATCAGCCGTAAT
TATTATAATTGGCTTGGTGCTGGCTACTATTGTGGCCATGTACGTGCTGA
CCAACCAGAAACATAATTGAATACAGCAGCAATTGGCAAGCTGCTTACAT
AGAACTCGCGGCGATTGGCATGCCGCCTTAAAATTTTTATTTTATTTTTT
CTTTTCTTTTCCGAATCGGATTTTGTTTTTAATATTTCAAAAAAAAAAAA
AAAAAAAAAAAAATCTAGAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAA

SEQ ID NO:23 - 3′ UTR (of SEQ NO:18), without poly-A

ACTCGAGTATGTTACGTGCAAAGGTGATTGTCACCCCCCGAAAGACCATA
TTGTGACACACCCTCAGTATCACGCCCAAACATTTACAGCCGCGGTGTCA
AAAACCGCGTGGACGTGGTTAACATCCCTGCTGGGAGGATCAGCCGTAAT
TATTATAATTGGCTTGGTGCTGGCTACTATTGTGGCCATGTACGTGCTGA
CCAACCAGAAACATAATTGAATACAGCAGCAATTGGCAAGCTGCTTACAT
AGAACTCGCGGCGATTGGCATGCCGCCTTAAAATTTTTATTTTATTTTTT
CTTTTCTTTTCCGAATCGGATTTTGTTTTTAATATTTC

SEQ ID NO:24 - Transgene (nucleic acid sequence; mARM3015/SEQ ID NO:18; codon-optimized)

ATGTTCGTCTTCCTGGTCCTGCTGCCTCTGGTCTCCTCACAGTGCGTCAA
TCTGACAACTCGGACTCAGCTGCCACCTGCTTATACTAATAGCTTCACCA
GAGGCGTGTACTATCCTGACAAGGTGTTTAGAAGCTCCGTGCTGCACTCT
ACACAGGATCTGTTTCTGCCATTCTTTAGCAACGTGACCTGGTTCCACGC
CATCCACGTGAGCGGCACCAATGGCACAAAGCGGTTCGACAATCCCGTGC
TGCCTTTTAACGATGGCGTGTACTTCGCCTCTACCGAGAAGTCCAACATC
ATCAGAGGCTGGATCTTTGGCACCACACTGGACTCCAAGACACAGTCTCT
GCTGATCGTGAACAATGCCACCAACGTGGTCATCAAGGTGTGCGAGTTCC
AGTTTTGTAATGATCCCTTCCTGGGCGTGTACTATCACAAGAACAATAAG
AGCTGGATGGAGTCCGAGTTTAGAGTGTATTCTAGCGCCAACAACTGCAC
ATTTGAGTACGTGAGCCAGCCTTTCCTGATGGACCTGGAGGGCAAGCAGG
GCAATTTCAAGAACCTGAGGGAGTTCGTGTTTAAGAATATCGACGGCTAC
TTCAAAATCTACTCTAAGCACACCCCCATCAACCTGGTGCGCGACCTGCC
TCAGGGCTTCAGCGCCCTGGAGCCCCTGGTGGATCTGCCTATCGGCATCA
ACATCACCCGGTTTCAGACACTGCTGGCCCTGCACAGAAGCTACCTGACA
CCCGGCGACTCCTCTAGCGGATGGACCGCCGGCGCTGCCGCCTACTATGT
GGGCTACCTCCAGCCCCGGACCTTCCTGCTGAAGTACAACGAGAATGGCA
CCATCACAGACGCAGTGGATTGCGCCCTGGACCCCCTGAGCGAGACAAAG
TGTACACTGAAGTCCTTTACCGTGGAGAAGGGCATCTATCAGACATCCAA
TTTCAGGGTGCAGCCAACCGAGTCTATCGTGCGCTTTCCTAATATCACAA
ACCTGTGCCCATTTGGCGAGGTGTTCAACGCAACCCGCTTCGCCAGCGTG
TACGCCTGGAATAGGAAGCGGATCAGCAACTGCGTGGCCGACTATAGCGT
GCTGTACAACTCCGCCTCTTTCAGCACCTTTAAGTGCTATGGCGTGTCCC
CCACAAAGCTGAATGACCTGTGCTTTACCAACGTCTACGCCGATTCTTTC
GTGATCAGGGGCGACGAGGTGCGCCAGATCGCCCCCGGCCAGACAGGCAA
GATCGCAGACTACAATTATAAGCTGCCAGACGATTTCACCGGCTGCGTGA
TCGCCTGGAACAGCAACAATCTGGATTCCAAAGTGGGCGGCAACTACAAT
TATCTGTACCGGCTGTTTAGAAAGAGCAATCTGAAGCCCTTCGAGAGGGA
CATCTCTACAGAAATCTACCAGGCCGGCAGCACCCCTTGCAATGGCGTGG
AGGGCTTTAACTGTTATTTCCCACTCCAGTCCTACGGCTTCCAGCCCACA
AACGGCGTGGGCTATCAGCCTTACCGCGTGGTGGTGCTGAGCTTTGAGCT
GCTGCACGCCCCAGCAACAGTGTGCGGCCCCAAGAAGTCCACCAATCTGG
TGAAGAACAAGTGCGTGAACTTCAACTTCAACGGCCTGACCGGCACAGGC
GTGCTGACCGAGTCCAACAAGAAGTTCCTGCCATTTCAGCAGTTCGGCAG
GGACATCGCAGATACCACAGACGCCGTGCGCGACCCACAGACCCTGGAGA
TCCTGGACATCACACCCTGCTCTTTCGGCGGCGTGAGCGTGATCACACCC
GGCACCAATACAAGCAACCAGGTGGCCGTGCTGTATCAGGACGTGAATTG
TACCGAGGTGCCCGTGGCTATCCACGCCGATCAGCTGACCCCAACATGGC
GGGTGTACAGCACCGGCTCCAACGTCTTCCAGACAAGAGCCGGATGCCTG
ATCGGAGCAGAGCACGTGAACAATTCCTATGAGTGCGACATCCCAATCGG
CGCCGGCATCTGTGCCTCTTACCAGACCCAGACAAACTCTCCCAGACGGG
CCCGGAGCGTGGCCTCCCAGTCTATCATCGCCTATACCATGTCCCTGGGC
GCCGAGAACAGCGTGGCCTACTCTAACAATAGCATCGCCATCCCAACCAA
CTTCACAATCTCTGTGACCACAGAGATCCTGCCCGTGTCCATGACCAAGA
CATCTGTGGACTGCACAATGTATATCTGTGGCGATTCTACCGAGTGCAGC
AACCTGCTGCTCCAGTACGGCAGCTTTTGTACCCAGCTGAATAGAGCCCT
GACAGGCATCGCCGTGGAGCAGGATAAGAACACACAGGAGGTGTTCGCCC
AGGTGAAGCAAATCTACAAGACCCCCCCTATCAAGGACTTTGGCGGCTTC
AATTTTTCCCAGATCCTGCCTGATCCATCCAAGCCTTCTAAGCGGAGCTT
TATCGAGGACCTGCTGTTCAACAAGGTGACCCTGGCCGATGCCGGCTTCA
TCAAGCAGTATGGCGATTGCCTGGGCGACATCGCAGCCAGGGACCTGATC
TGCGCCCAGAAGTTTAATGGCCTGACCGTGCTGCCACCCCTGCTGACAGA
TGAGATGATCGCACAGTACACAAGCGCCCTGCTGGCCGGCACCATCACAT
CCGGATGGACCTTCGGCGCAGGAGCCGCCCTCCAGATCCCCTTTGCCATG
CAGATGGCCTATAGGTTCAACGGCATCGGCGTGACCCAGAATGTGCTGTA
CGAGAACCAGAAGCTGATCGCCAATCAGTTTAACTCCGCCATCGGCAAGA
TCCAGGACAGCCTGTCCTCTACAGCCAGCGCCCTGGGCAAGCTCCAGGAT
GTGGTGAATCAGAACGCCCAGGCCCTGAATACCCTGGTGAAGCAGCTGAG
CAGCAACTTCGGCGCCATCTCTAGCGTGCTGAATGACATCCTGAGCCGGC
TGGACAAGGTGGAGGCAGAGGTGCAGATCGACCGGCTGATCACCGGCCGG
CTCCAGAGCCTCCAGACCTATGTGACACAGCAGCTGATCAGGGCCGCCGA
GATCAGGGCCAGCGCCAATCTGGCAGCAACCAAGATGTCCGAGTGCGTGC
TGGGCCAGTCTAAGAGAGTGGACTTTTGTGGCAAGGGCTATCACCTGATG
TCCTTCCCTCAGTCTGCCCCACACGGCGTGGTGTTTCTGCACGTGACCTA
CGTGCCCGCCCAGGAGAAGAACTTCACCACAGCCCCTGCCATCTGCCACG
ATGGCAAGGCCCACTTTCCAAGGGAGGGCGTGTTCGTGTCCAACGGCACC
CACTGGTTTGTGACACAGCGCAATTTCTACGAGCCCCAGATCATCACCAC
AGACAACACCTTCGTGAGCGGCAACTGTGACGTGGTCATCGGCATCGTGA
ACAATACCGTGTATGATCCACTCCAGCCCGAGCTGGACAGCTTTAAGGAG
GAGCTGGATAAGTATTTCAAGAATCACACCTCCCCTGACGTGGATCTGGG
CGACATCAGCGGCATCAATGCCTCCGTGGTGAACATCCAGAAGGAGATCG
ACCGCCTGAACGAGGTGGCTAAGAATCTGAACGAGAGCCTGATCGACCTC
CAGGAGCTGGGCAAGTATGAGCAGTACATCAAGTGGCCCTGGTACATCTG
GCTGGGCTTCATCGCCGGCCTGATCGCCATCGTGATGGTGACCATCATGC
TGTGCTGTATGACATCCTGCTGTTCTTGCCTGAAGGGCTGCTGTAGCTGT
GGCTCCTGCTGTAAGTTTGACGAGGATGACTCTGAACCTGTGCTGAAGGG
CGTGAAGCTGCATTACACCTAA

SEQ ID NO:25 - Transgene (nucleic acid sequence; mARM3015/SEQ ID NO:18; not codon-optimized)

ATGTTTGTTTTTCTTGTTTTATTGCCACTAGTCTCTAGTCAGTGTGTTAA
TCTTACAACCAGAACTCAATTACCCCCTGCATACACTAATTCTTTCACAC
GTGGTGTTTATTACCCTGACAAAGTTTTCAGATCCTCAGTTTTACATTCA
ACTCAGGACTTGTTCTTACCTTTCTTTTCCAATGTTACTTGGTTCCATGC
TATACATGTCTCTGGGACCAATGGTACTAAGAGGTTTGATAACCCTGTCC
TACCATTTAATGATGGTGTTTATTTTGCTTCCACTGAGAAGTCTAACATA
ATAAGAGGCTGGATTTTTGGTACTACTTTAGATTCGAAGACCCAGTCCCT
ACTTATTGTTAATAACGCTACTAATGTTGTTATTAAAGTCTGTGAATTTC
AATTTTGTAATGATCCATTTTTGGGTGTTTATTACCACAAAAACAACAAA
AGTTGGATGGAAAGTGAGTTCAGAGTTTATTCTAGTGCGAATAATTGCAC
TTTTGAATATGTCTCTCAGCCTTTTCTTATGGACCTTGAAGGAAAACAGG
GTAATTTCAAAAATCTTAGGGAATTTGTGTTTAAGAATATTGATGGTTAT
TTTAAAATATATTCTAAGCACACGCCTATTAATTTAGTGCGTGATCTCCC
TCAGGGTTTTTCGGCTTTAGAACCATTGGTAGATTTGCCAATAGGTATTA
ACATCACTAGGTTTCAAACTTTACTTGCTTTACATAGAAGTTATTTGACT
CCTGGTGATTCTTCTTCAGGTTGGACAGCTGGTGCTGCAGCTTATTATGT
GGGTTATCTTCAACCTAGGACTTTTCTATTAAAATATAATGAAAATGGAA
CCATTACAGATGCTGTAGACTGTGCACTTGACCCTCTCTCAGAAACAAAG
TGTACGTTGAAATCCTTCACTGTAGAAAAAGGAATCTATCAAACTTCTAA
CTTTAGAGTCCAACCAACAGAATCTATTGTTAGATTTCCTAATATTACAA
ACTTGTGCCCTTTTGGTGAAGTTTTTAACGCCACCAGATTTGCATCTGTT
TATGCTTGGAACAGGAAGAGAATCAGCAACTGTGTTGCTGATTATTCTGT
CCTATATAATTCCGCATCATTTTCCACTTTTAAGTGTTATGGAGTGTCTC
CTACTAAATTAAATGATCTCTGCTTTACTAATGTCTATGCAGATTCATTT
GTAATTAGAGGTGATGAAGTCAGACAAATCGCTCCAGGGCAAACTGGAAA
GATTGCTGATTATAATTATAAATTACCAGATGATTTTACAGGCTGCGTTA
TAGCTTGGAATTCTAACAATCTTGATTCTAAGGTTGGTGGTAATTATAAT
TACCTGTATAGATTGTTTAGGAAGTCTAATCTCAAACCTTTTGAGAGAGA
TATTTCAACTGAAATCTATCAGGCCGGTAGCACACCTTGTAATGGTGTTG
AAGGTTTTAATTGTTACTTTCCTTTACAATCATATGGTTTCCAACCCACT
AATGGTGTTGGTTACCAACCATACAGAGTAGTAGTACTTTCTTTTGAACT
TCTACATGCACCAGCAACTGTTTGTGGACCTAAAAAGTCTACTAATTTGG
TTAAAAACAAATGTGTCAATTTCAACTTCAATGGTTTAACAGGCACAGGT
GTTCTTACTGAGTCTAACAAAAAGTTTCTGCCTTTCCAACAATTTGGCAG
AGACATTGCTGACACTACTGATGCTGTCCGTGATCCACAGACACTTGAGA
TTCTTGACATTACACCATGTTCTTTTGGTGGTGTCAGTGTTATAACACCA
GGAACAAATACTTCTAACCAGGTTGCTGTTCTTTATCAGGATGTTAACTG
CACAGAAGTCCCTGTTGCTATTCATGCAGATCAACTTACTCCTACTTGGC
GTGTTTATTCTACAGGTTCTAATGTTTTTCAAACACGTGCAGGCTGTTTA
ATAGGGGCTGAACATGTCAACAACTCATATGAGTGTGACATACCCATTGG
TGCAGGTATATGCGCTAGTTATCAGACTCAGACTAATTCTCCTCGGCGGG
CACGTAGTGTAGCTAGTCAATCCATCATTGCCTACACTATGTCACTTGGT
GCAGAAAATTCAGTTGCTTACTCTAATAACTCTATTGCCATACCCACAAA
TTTTACTATTAGTGTTACCACAGAAATTCTACCAGTGTCTATGACCAAGA
CATCAGTAGATTGTACAATGTACATTTGTGGTGATTCAACTGAATGCAGC
AATCTTTTGTTGCAATATGGCAGTTTTTGTACACAATTAAACCGTGCTTT
AACTGGAATAGCTGTTGAACAAGACAAAAACACCCAAGAAGTTTTTGCAC
AAGTCAAACAAATTTACAAAACACCACCAATTAAAGATTTTGGTGGTTTT
AATTTTTCACAAATATTACCAGATCCATCAAAACCAAGCAAGAGGTCATT
TATTGAAGATCTACTTTTCAACAAAGTGACACTTGCAGATGCTGGCTTCA
TCAAACAATATGGTGATTGCCTTGGTGATATTGCTGCTAGAGACCTCATT
TGTGCACAAAAGTTTAACGGCCTTACTGTTTTGCCACCTTTGCTCACAGA
TGAAATGATTGCTCAATACACTTCTGCACTGTTAGCGGGTACAATCACTT
CTGGTTGGACCTTTGGTGCAGGTGCTGCATTACAAATACCATTTGCTATG
CAAATGGCTTATAGGTTTAATGGTATTGGAGTTACACAGAATGTTCTCTA
TGAGAACCAAAAATTGATTGCCAACCAATTTAATAGTGCTATTGGCAAAA
TTCAAGACTCACTTTCTTCCACAGCAAGTGCACTTGGAAAACTTCAAGAT
GTGGTCAACCAAAATGCACAAGCTTTAAACACGCTTGTTAAACAACTTAG
CTCCAATTTTGGTGCAATTTCAAGTGTTTTAAATGATATCCTTTCACGTC
TTGACAAAGTTGAGGCTGAAGTGCAAATTGATAGGTTGATCACAGGCAGA
CTTCAAAGTTTGCAGACATATGTGACTCAACAATTAATTAGAGCTGCAGA
AATCAGAGCTTCTGCTAATCTTGCTGCTACTAAAATGTCAGAGTGTGTAC
TTGGACAATCAAAAAGAGTTGATTTTTGTGGAAAGGGCTATCATCTTATG
TCCTTCCCTCAGTCAGCACCTCATGGTGTAGTCTTCTTGCATGTGACTTA
TGTCCCTGCACAAGAAAAGAACTTCACAACTGCTCCTGCCATTTGTCATG
ATGGAAAAGCACACTTTCCTCGTGAAGGTGTCTTTGTTTCAAATGGCACA
CACTGGTTTGTAACACAAAGGAATTTTTATGAACCACAAATCATTACTAC
AGACAACACATTTGTGTCTGGTAACTGTGATGTTGTAATAGGAATTGTCA
ACAACACAGTTTATGATCCTTTGCAACCTGAATTAGACTCATTCAAGGAG
GAGTTAGATAAATATTTTAAGAATCATACATCACCAGATGTTGATTTAGG
TGACATCTCTGGCATTAATGCTTCAGTTGTAAACATTCAAAAAGAAATTG
ACCGCCTCAATGAGGTTGCCAAGAATTTAAATGAATCTCTCATCGATCTC
CAAGAACTTGGAAAGTATGAGCAGTATATAAAATGGCCATGGTACATTTG
GCTAGGTTTTATAGCTGGCTTGATTGCCATAGTAATGGTGACAATTATGC
TTTGCTGTATGACCAGTTGCTGTAGTTGTCTCAAGGGCTGTTGTTCTTGT
GGATCCTGCTGCAAATTTGATGAAGACGACTCTGAGCCAGTGCTCAAAGG
AGTCAAATTACATTACACATAA

SEQ ID NO:26 - Transgene (amino acid sequence; mARM3015/SEQ ID NO:18)

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

SEQ ID NO:27 - Replicon sequence including SEQ ID NO:19, SEQ ID NO:20, SEQ ID NO:21, and SEQ ID NO:22 (with poly-A)

ATGGGCGGCGCATGAGAGAAGCCCAGACCAATTACCTACCCAAAATGGAG
AAAGTTCACGTTGACATCGAGGAAGACAGCCCATTCCTCAGAGCTTTGCA
GCGGAGCTTCCCGCAGTTTGAGGTAGAAGCCAAGCAGGTCACTGATAATG
ACCATGCTAATGCCAGAGCGTTTTCGCATCTGGCTTCAAAACTGATCGAA
ACGGAGGTGGACCCATCCGACACGATCCTTGACATTGGAAGTGCGCCCGC
CCGCAGAATGTATTCTAAGCACAAGTATCATTGTATCTGTCCGATGAGAT
GTGCGGAAGATCCGGACAGATTGTATAAGTATGCAACTAAGCTGAAGAAA
AACTGTAAGGAAATAACTGATAAGGAATTGGACAAGAAAATGAAGGAGCT
GGCCGCCGTCATGAGCGACCCTGACCTGGAAACTGAGACTATGTGCCTCC
ACGACGACGAGTCGTGTCGCTACGAAGGGCAAGTCGCTGTTTACCAGGAT
GTATACGCCGTCGACGGCCCCACCAGCCTGTACCACCAGGCCAACAAGGG
CGTGAGGGTGGCCTACTGGATCGGCTTCGACACCACACCCTTCATGTTCA
AGAACCTGGCCGGCGCCTACCCCAGCTACAGCACCAACTGGGCCGACGAG
ACCGTGCTGACCGCCAGGAACATCGGCCTGTGCAGCAGCGACGTGATGGA
GAGGAGCCGGAGAGGCATGAGCATCCTGAGGAAGAAATACCTGAAGCCCA
GCAACAACGTGCTGTTCAGCGTGGGCAGCACCATCTACCACGAGAAGAGG
GACCTGCTCAGGAGCTGGCACCTGCCCAGCGTGTTCCACCTGAGGGGCAA
GCAGAACTACACCTGCAGGTGCGAGACCATCGTGAGCTGCGACGGCTACG
TGGTGAAGAGGATCGCCATCAGCCCCGGCCTGTACGGCAAGCCCAGCGGC
TACGCCGCTACAATGCACAGGGAGGGCTTCCTGTGCTGCAAGGTGACCGA
CACCCTGAACGGCGAGAGGGTGAGCTTCCCCGTGTGCACCTACGTGCCCG
CCACCCTGTGCGACCAGATGACCGGCATCCTGGCCACCGACGTGAGCGCC
GACGACGCCCAGAAGCTGCTCGTGGGCCTGAACCAGAGGATCGTGGTCAA
CGGCAGGACCCAGAGGAACACCAACACAATGAAGAACTACCTGCTGCCCG
TGGTGGCCCAGGCTTTCGCCAGGTGGGCCAAGGAGTACAAGGAGGACCAG
GAAGACGAGAGGCCCCTGGGCCTGAGGGACAGGCAGCTGGTGATGGGCTG
CTGCTGGGCCTTCAGGCGGCACAAGATCACCAGCATCTACAAGAGGCCCG
ACACCCAGACCATCATCAAGGTGAACAGCGACTTCCACAGCTTCGTGCTG
CCCAGGATCGGCAGCAACACCCTGGAGATCGGCCTGAGGACCCGGATCAG
GAAGATGCTGGAGGAACACAAGGAGCCCAGCCCACTGATCACCGCCGAGG
ACGTGCAGGAGGCCAAGTGCGCTGCCGACGAGGCCAAGGAGGTGAGGGAG
GCCGAGGAACTGAGGGCCGCCCTGCCACCCCTGGCTGCCGACGTGGAGGA
ACCCACCCTGGAAGCCGACGTGGACCTGATGCTGCAGGAGGCCGGCGCCG
GAAGCGTGGAGACACCCAGGGGCCTGATCAAGGTGACCAGCTACGACGGC
GAGGACAAGATCGGCAGCTACGCCGTGCTGAGCCCACAGGCCGTGCTGAA
GTCCGAGAAGCTGAGCTGCATCCACCCACTGGCCGAGCAGGTGATCGTGA
TCACCCACAGCGGCAGGAAGGGCAGGTACGCCGTGGAGCCCTACCACGGC
AAGGTGGTCGTGCCCGAGGGCCACGCCATCCCCGTGCAGGACTTCCAGGC
CCTGAGCGAGAGCGCCACCATCGTGTACAACGAGAGGGAGTTCGTGAACA
GGTACCTGCACCATATCGCCACCCACGGCGGAGCCCTGAACACCGACGAG
GAATACTACAAGACCGTGAAGCCCAGCGAGCACGACGGCGAGTACCTGTA
CGACATCGACAGGAAGCAGTGCGTGAAGAAAGAGCTGGTGACCGGCCTGG
GACTGACCGGCGAGCTGGTGGACCCACCCTTCCACGAGTTCGCCTACGAG
AGCCTGAGGACCAGACCCGCCGCTCCCTACCAGGTGCCCACCATCGGCGT
GTACGGCGTGCCCGGCAGCGGAAAGAGCGGCATCATCAAGAGCGCCGTGA
CCAAGAAAGACCTGGTGGTCAGCGCCAAGAAAGAGAACTGCGCCGAGATC
ATCAGGGACGTGAAGAAGATGAAAGGCCTGGACGTGAACGCGCGCACCGT
GGACAGCGTGCTGCTGAACGGCTGCAAGCACCCCGTGGAGACCCTGTACA
TCGACGAGGCCTTCGCTTGCCACGCCGGCACCCTGAGGGCCCTGATCGCC
ATCATCAGGCCCAAGAAAGCCGTGCTGTGCGGCGACCCCAAGCAGTGCGG
CTTCTTCAACATGATGTGCCTGAAGGTGCACTTCAACCACGAGATCTGCA
CCCAGGTGTTCCACAAGAGCATCAGCAGGCGGTGCACCAAGAGCGTGACC
AGCGTCGTGAGCACCCTGTTCTACGACAAGAAAATGAGGACCACCAACCC
CAAGGAGACCAAAATCGTGATCGACACCACAGGCAGCACCAAGCCCAAGC
AGGACGACCTGATCCTGACCTGCTTCAGGGGCTGGGTGAAGCAGCTGCAG
ATCGACTACAAGGGCAACGAGATCATGACCGCCGCTGCCAGCCAGGGCCT
GACCAGGAAGGGCGTGTACGCCGTGAGGTACAAGGTGAACGAGAACCCAC
TGTACGCTCCCACCAGCGAGCACGTGAACGTGCTGCTGACCAGGACCGAG
GACAGGATCGTGTGGAAGACCCTGGCCGGCGACCCCTGGATCAAGACCCT
GACCGCCAAGTACCCCGGCAACTTCACCGCCACCATCGAAGAGTGGCAGG
CCGAGCACGACGCCATCATGAGGCACATCCTGGAGAGGCCCGACCCCACC
GACGTGTTCCAGAACAAGGCCAACGTGTGCTGGGCCAAGGCCCTGGTGCC
CGTGCTGAAGACCGCCGGCATCGACATGACCACAGAGCAGTGGAACACCG
TGGACTACTTCGAGACCGACAAGGCCCACAGCGCCGAGATCGTGCTGAAC
CAGCTGTGCGTGAGGTTCTTCGGCCTGGACCTGGACAGCGGCCTGTTCAG
CGCCCCCACCGTGCCACTGAGCATCAGGAACAACCACTGGGACAACAGCC
CCAGCCCAAACATGTACGGCCTGAACAAGGAGGTGGTCAGGCAGCTGAGC
AGGCGGTACCCACAGCTGCCCAGGGCCGTGGCCACCGGCAGGGTGTACGA
CATGAACACCGGCACCCTGAGGAACTACGACCCCAGGATCAACCTGGTGC
CCGTGAACAGGCGGCTGCCCCACGCCCTGGTGCTGCACCACAACGAGCAC
CCACAGAGCGACTTCAGCTCCTTCGTGAGCAAGCTGAAAGGCAGGACCGT
GCTGGTCGTGGGCGAGAAGCTGAGCGTGCCCGGCAAGATGGTGGACTGGC
TGAGCGACAGGCCCGAGGCCACCTTCCGGGCCAGGCTGGACCTCGGCATC
CCCGGCGACGTGCCCAAGTACGACATCATCTTCGTGAACGTCAGGACCCC
ATACAAGTACCACCATTACCAGCAGTGCGAGGACCACGCCATCAAGCTGA
GCATGCTGACCAAGAAGGCCTGCCTGCACCTGAACCCCGGAGGCACCTGC
GTGAGCATCGGCTACGGCTACGCCGACAGGGCCAGCGAGAGCATCATTGG
CGCCATCGCCAGGCTGTTCAAGTTCAGCAGGGTGTGCAAACCCAAGAGCA
GCCTGGAGGAAACCGAGGTGCTGTTCGTGTTCATCGGCTACGACCGGAAG
GCCAGGACCCACAACCCCTACAAGCTGAGCAGCACCCTGACAAACATCTA
CACCGGCAGCAGGCTGCACGAGGCCGGCTGCGCCCCCAGCTACCACGTGG
TCAGGGGCGATATCGCCACCGCCACCGAGGGCGTGATCATCAACGCTGCC
AACAGCAAGGGCCAGCCCGGAGGCGGAGTGTGCGGCGCCCTGTACAAGAA
GTTCCCCGAGAGCTTCGACCTGCAGCCCATCGAGGTGGGCAAGGCCAGGC
TGGTGAAGGGCGCCGCTAAGCACATCATCCACGCCGTGGGCCCCAACTTC
AACAAGGTGAGCGAGGTGGAAGGCGACAAGCAGCTGGCCGAAGCCTACGA
GAGCATCGCCAAGATCGTGAACGACAATAACTACAAGAGCGTGGCCATCC
CACTGCTCAGCACCGGCATCTTCAGCGGCAACAAGGACAGGCTGACCCAG
AGCCTGAACCACCTGCTCACCGCCCTGGACACCACCGATGCCGACGTGGC
CATCTACTGCAGGGACAAGAAGTGGGAGATGACCCTGAAGGAGGCCGTGG
CCAGGCGGGAGGCCGTGGAAGAGATCTGCATCAGCGACGACTCCAGCGTG
ACCGAGCCCGACGCCGAGCTGGTGAGGGTGCACCCCAAGAGCTCCCTGGC
CGGCAGGAAGGGCTACAGCACCAGCGACGGCAAGACCTTCAGCTACCTGG
AGGGCACCAAGTTCCACCAGGCCGCTAAGGACATCGCCGAGATCAACGCT
ATGTGGCCCGTGGCCACCGAGGCCAACGAGCAGGTGTGCATGTACATCCT
GGGCGAGAGCATGTCCAGCATCAGGAGCAAGTGCCCCGTGGAGGAAAGCG
AGGCCAGCACACCACCCAGCACCCTGCCCTGCCTGTGCATCCACGCTATG
ACACCCGAGAGGGTGCAGCGGCTGAAGGCCAGCAGGCCCGAGCAGATCAC
CGTGTGCAGCTCCTTCCCACTGCCCAAGTACAGGATCACCGGCGTGCAGA
AGATCCAGTGCAGCCAGCCCATCCTGTTCAGCCCAAAGGTGCCCGCCTAC
ATCCACCCCAGGAAGTACCTGGTGGAGACCCCACCCGTGGACGAGACACC
CGAGCCAAGCGCCGAGAACCAGAGCACCGAGGGCACACCCGAGCAGCCAC
CCCTGATCACCGAGGACGAGACAAGGACCCGGACCCCAGAGCCCATCATT
ATCGAGGAAGAGGAAGAGGACAGCATCAGCCTGCTGAGCGACGGCCCCAC
CCACCAGGTGCTGCAGGTGGAGGCCGACATCCACGGCCCACCCAGCGTGT
CCAGCTCCAGCTGGAGCATCCCACACGCCAGCGACTTCGACGTGGACAGC
CTGAGCATCCTGGACACCCTGGAGGGCGCCAGCGTGACCTCCGGCGCCAC
CAGCGCCGAGACCAACAGCTACTTCGCCAAGAGCATGGAGTTCCTGGCCA
GGCCCGTGCCAGCTCCCAGGACCGTGTTCAGGAACCCACCCCACCCAGCT
CCCAGGACCAGGACCCCAAGCCTGGCTCCCAGCAGGGCCTGCAGCAGGAC
CAGCCTGGTGAGCACCCCACCCGGCGTGAACAGGGTGATCACCAGGGAGG
AACTGGAGGCCCTGACACCCAGCAGGACCCCCAGCAGGTCCGTGAGCAGG
ACTAGTCTGGTGTCCAACCCACCCGGCGTGAACAGGGTGATCACCAGGGA
GGAATTCGAGGCCTTCGTGGCCCAGCAACAGAGACGGTTCGACGCCGGCG
CCTACATCTTCAGCAGCGACACCGGCCAGGGACACCTGCAGCAAAAGAGC
GTGAGGCAGACCGTGCTGAGCGAGGTGGTGCTGGAGAGGACCGAGCTGGA
AATCAGCTACGCCCCCAGGCTGGACCAGGAGAAGGAGGAACTGCTCAGGA
AGAAACTGCAGCTGAACCCCACCCCAGCCAACAGGAGCAGGTACCAGAGC
AGGAAGGTGGAGAACATGAAGGCCATCACCGCCAGGCGGATCCTGCAGGG
CCTGGGACACTACCTGAAGGCCGAGGGCAAGGTGGAGTGCTACAGGACCC
TGCACCCCGTGCCACTGTACAGCTCCAGCGTGAACAGGGCCTTCTCCAGC
CCCAAGGTGGCCGTGGAGGCCTGCAACGCTATGCTGAAGGAGAACTTCCC
CACCGTGGCCAGCTACTGCATCATCCCCGAGTACGACGCCTACCTGGACA
TGGTGGACGGCGCCAGCTGCTGCCTGGACACCGCCAGCTTCTGCCCCGCC
AAGCTGAGGAGCTTCCCCAAGAAACACAGCTACCTGGAGCCCACCATCAG
GAGCGCCGTGCCCAGCGCCATCCAGAACACCCTGCAGAACGTGCTGGCCG
CTGCCACCAAGAGGAACTGCAACGTGACCCAGATGAGGGAGCTGCCCGTG
CTGGACAGCGCTGCCTTCAACGTGGAGTGCTTCAAGAAATACGCCTGCAA
CAACGAGTACTGGGAGACCTTCAAGGAGAACCCCATCAGGCTGACCGAAG
AGAACGTGGTGAACTACATCACCAAGCTGAAGGGCCCCAAGGCCGCTGCC
CTGTTCGCTAAGACCCACAACCTGAACATGCTGCAGGACATCCCAATGGA
CAGGTTCGTGATGGACCTGAAGAGGGACGTGAAGGTGACACCCGGCACCA
AGCACACCGAGGAGAGGCCCAAGGTGCAGGTGATCCAGGCCGCTGACCCA
CTGGCCACCGCCTACCTGTGCGGCATCCACAGGGAGCTGGTGAGGCGGCT
GAACGCCGTGCTGCTGCCCAACATCCACACCCTGTTCGACATGAGCGCCG
AGGACTTCGACGCCATCATCGCCGAGCACTTCCAGCCCGGCGACTGCGTG
CTGGAGACCGACATCGCCAGCTTCGACAAGAGCGAGGATGACGCTATGGC
CCTGACCGCTCTGATGATCCTGGAGGACCTGGGCGTGGACGCCGAGCTGC
TCACCCTGATCGAGGCTGCCTTCGGCGAGATCAGCTCCATCCACCTGCCC
ACCAAGACCAAGTTCAAGTTCGGCGCTATGATGAAAAGCGGAATGTTCCT
GACCCTGTTCGTGAACACCGTGATCAACATTGTGATCGCCAGCAGGGTGC
TGCGGGAGAGGCTGACCGGCAGCCCCTGCGCTGCCTTCATCGGCGACGAC
AACATCGTGAAGGGCGTGAAAAGCGACAAGCTGATGGCCGACAGGTGCGC
CACCTGGCTGAACATGGAGGTGAAGATCATCGACGCCGTGGTGGGCGAGA
AGGCCCCCTACTTCTGCGGCGGATTCATCCTGTGCGACAGCGTGACCGGC
ACCGCCTGCAGGGTGGCCGACCCCCTGAAGAGGCTGTTCAAGCTGGGCAA
GCCACTGGCCGCTGACGATGAGCACGACGATGACAGGCGGAGGGCCCTGC
ACGAGGAAAGCACCAGGTGGAACAGGGTGGGCATCCTGAGCGAGCTGTGC
AAGGCCGTGGAGAGCAGGTACGAGACCGTGGGCACCAGCATCATCGTGAT
GGCTATGACCACACTGGCCAGCTCCGTCAAGAGCTTCTCCTACCTGAGGG
GGGCCCCTATAACTCTCTACGGCTAACCTGAATGGACTACGACATAGTCT
AGTCCGCCAAGGCCGCCACCACTCGAGTATGTTACGTGCAAAGGTGATTG
TCACCCCCCGAAAGACCATATTGTGACACACCCTCAGTATCACGCCCAAA
CATTTACAGCCGCGGTGTCAAAAACCGCGTGGACGTGGTTAACATCCCTG
CTGGGAGGATCAGCCGTAATTATTATAATTGGCTTGGTGCTGGCTACTAT
TGTGGCCATGTACGTGCTGACCAACCAGAAACATAATTGAATACAGCAGC
AATTGGCAAGCTGCTTACATAGAACTCGCGGCGATTGGCATGCCGCCTTA
AAATTTTTATTTTATTTTTTCTTTTCTTTTCCGAATCGGATTTTGTTTTT
AATATTTCAAAAAAAAAAAAAAAAAAAAAAAAATCTAGAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAA

SEQ ID NO:28 - Replicon sequence including SEQ ID NO:19, SEQ ID NO:20, SEQ ID NO:21, and SEQ ID NO:23 (without poly-A)

ATGGGCGGCGCATGAGAGAAGCCCAGACCAATTACCTACCCAAAATGGAG
AAAGTTCACGTTGACATCGAGGAAGACAGCCCATTCCTCAGAGCTTTGCA
GCGGAGCTTCCCGCAGTTTGAGGTAGAAGCCAAGCAGGTCACTGATAATG
ACCATGCTAATGCCAGAGCGTTTTCGCATCTGGCTTCAAAACTGATCGAA
ACGGAGGTGGACCCATCCGACACGATCCTTGACATTGGAAGTGCGCCCGC
CCGCAGAATGTATTCTAAGCACAAGTATCATTGTATCTGTCCGATGAGAT
GTGCGGAAGATCCGGACAGATTGTATAAGTATGCAACTAAGCTGAAGAAA
AACTGTAAGGAAATAACTGATAAGGAATTGGACAAGAAAATGAAGGAGCT
GGCCGCCGTCATGAGCGACCCTGACCTGGAAACTGAGACTATGTGCCTCC
ACGACGACGAGTCGTGTCGCTACGAAGGGCAAGTCGCTGTTTACCAGGAT
GTATACGCCGTCGACGGCCCCACCAGCCTGTACCACCAGGCCAACAAGGG
CGTGAGGGTGGCCTACTGGATCGGCTTCGACACCACACCCTTCATGTTCA
AGAACCTGGCCGGCGCCTACCCCAGCTACAGCACCAACTGGGCCGACGAG
ACCGTGCTGACCGCCAGGAACATCGGCCTGTGCAGCAGCGACGTGATGGA
GAGGAGCCGGAGAGGCATGAGCATCCTGAGGAAGAAATACCTGAAGCCCA
GCAACAACGTGCTGTTCAGCGTGGGCAGCACCATCTACCACGAGAAGAGG
GACCTGCTCAGGAGCTGGCACCTGCCCAGCGTGTTCCACCTGAGGGGCAA
GCAGAACTACACCTGCAGGTGCGAGACCATCGTGAGCTGCGACGGCTACG
TGGTGAAGAGGATCGCCATCAGCCCCGGCCTGTACGGCAAGCCCAGCGGC
TACGCCGCTACAATGCACAGGGAGGGCTTCCTGTGCTGCAAGGTGACCGA
CACCCTGAACGGCGAGAGGGTGAGCTTCCCCGTGTGCACCTACGTGCCCG
CCACCCTGTGCGACCAGATGACCGGCATCCTGGCCACCGACGTGAGCGCC
GACGACGCCCAGAAGCTGCTCGTGGGCCTGAACCAGAGGATCGTGGTCAA
CGGCAGGACCCAGAGGAACACCAACACAATGAAGAACTACCTGCTGCCCG
TGGTGGCCCAGGCTTTCGCCAGGTGGGCCAAGGAGTACAAGGAGGACCAG
GAAGACGAGAGGCCCCTGGGCCTGAGGGACAGGCAGCTGGTGATGGGCTG
CTGCTGGGCCTTCAGGCGGCACAAGATCACCAGCATCTACAAGAGGCCCG
ACACCCAGACCATCATCAAGGTGAACAGCGACTTCCACAGCTTCGTGCTG
CCCAGGATCGGCAGCAACACCCTGGAGATCGGCCTGAGGACCCGGATCAG
GAAGATGCTGGAGGAACACAAGGAGCCCAGCCCACTGATCACCGCCGAGG
ACGTGCAGGAGGCCAAGTGCGCTGCCGACGAGGCCAAGGAGGTGAGGGAG
GCCGAGGAACTGAGGGCCGCCCTGCCACCCCTGGCTGCCGACGTGGAGGA
ACCCACCCTGGAAGCCGACGTGGACCTGATGCTGCAGGAGGCCGGCGCCG
GAAGCGTGGAGACACCCAGGGGCCTGATCAAGGTGACCAGCTACGACGGC
GAGGACAAGATCGGCAGCTACGCCGTGCTGAGCCCACAGGCCGTGCTGAA
GTCCGAGAAGCTGAGCTGCATCCACCCACTGGCCGAGCAGGTGATCGTGA
TCACCCACAGCGGCAGGAAGGGCAGGTACGCCGTGGAGCCCTACCACGGC
AAGGTGGTCGTGCCCGAGGGCCACGCCATCCCCGTGCAGGACTTCCAGGC
CCTGAGCGAGAGCGCCACCATCGTGTACAACGAGAGGGAGTTCGTGAACA
GGTACCTGCACCATATCGCCACCCACGGCGGAGCCCTGAACACCGACGAG
GAATACTACAAGACCGTGAAGCCCAGCGAGCACGACGGCGAGTACCTGTA
CGACATCGACAGGAAGCAGTGCGTGAAGAAAGAGCTGGTGACCGGCCTGG
GACTGACCGGCGAGCTGGTGGACCCACCCTTCCACGAGTTCGCCTACGAG
AGCCTGAGGACCAGACCCGCCGCTCCCTACCAGGTGCCCACCATCGGCGT
GTACGGCGTGCCCGGCAGCGGAAAGAGCGGCATCATCAAGAGCGCCGTGA
CCAAGAAAGACCTGGTGGTCAGCGCCAAGAAAGAGAACTGCGCCGAGATC
ATCAGGGACGTGAAGAAGATGAAAGGCCTGGACGTGAACGCGCGCACCGT
GGACAGCGTGCTGCTGAACGGCTGCAAGCACCCCGTGGAGACCCTGTACA
TCGACGAGGCCTTCGCTTGCCACGCCGGCACCCTGAGGGCCCTGATCGCC
ATCATCAGGCCCAAGAAAGCCGTGCTGTGCGGCGACCCCAAGCAGTGCGG
CTTCTTCAACATGATGTGCCTGAAGGTGCACTTCAACCACGAGATCTGCA
CCCAGGTGTTCCACAAGAGCATCAGCAGGCGGTGCACCAAGAGCGTGACC
AGCGTCGTGAGCACCCTGTTCTACGACAAGAAAATGAGGACCACCAACCC
CAAGGAGACCAAAATCGTGATCGACACCACAGGCAGCACCAAGCCCAAGC
AGGACGACCTGATCCTGACCTGCTTCAGGGGCTGGGTGAAGCAGCTGCAG
ATCGACTACAAGGGCAACGAGATCATGACCGCCGCTGCCAGCCAGGGCCT
GACCAGGAAGGGCGTGTACGCCGTGAGGTACAAGGTGAACGAGAACCCAC
TGTACGCTCCCACCAGCGAGCACGTGAACGTGCTGCTGACCAGGACCGAG
GACAGGATCGTGTGGAAGACCCTGGCCGGCGACCCCTGGATCAAGACCCT
GACCGCCAAGTACCCCGGCAACTTCACCGCCACCATCGAAGAGTGGCAGG
CCGAGCACGACGCCATCATGAGGCACATCCTGGAGAGGCCCGACCCCACC
GACGTGTTCCAGAACAAGGCCAACGTGTGCTGGGCCAAGGCCCTGGTGCC
CGTGCTGAAGACCGCCGGCATCGACATGACCACAGAGCAGTGGAACACCG
TGGACTACTTCGAGACCGACAAGGCCCACAGCGCCGAGATCGTGCTGAAC
CAGCTGTGCGTGAGGTTCTTCGGCCTGGACCTGGACAGCGGCCTGTTCAG
CGCCCCCACCGTGCCACTGAGCATCAGGAACAACCACTGGGACAACAGCC
CCAGCCCAAACATGTACGGCCTGAACAAGGAGGTGGTCAGGCAGCTGAGC
AGGCGGTACCCACAGCTGCCCAGGGCCGTGGCCACCGGCAGGGTGTACGA
CATGAACACCGGCACCCTGAGGAACTACGACCCCAGGATCAACCTGGTGC
CCGTGAACAGGCGGCTGCCCCACGCCCTGGTGCTGCACCACAACGAGCAC
CCACAGAGCGACTTCAGCTCCTTCGTGAGCAAGCTGAAAGGCAGGACCGT
GCTGGTCGTGGGCGAGAAGCTGAGCGTGCCCGGCAAGATGGTGGACTGGC
TGAGCGACAGGCCCGAGGCCACCTTCCGGGCCAGGCTGGACCTCGGCATC
CCCGGCGACGTGCCCAAGTACGACATCATCTTCGTGAACGTCAGGACCCC
ATACAAGTACCACCATTACCAGCAGTGCGAGGACCACGCCATCAAGCTGA
GCATGCTGACCAAGAAGGCCTGCCTGCACCTGAACCCCGGAGGCACCTGC
GTGAGCATCGGCTACGGCTACGCCGACAGGGCCAGCGAGAGCATCATTGG
CGCCATCGCCAGGCTGTTCAAGTTCAGCAGGGTGTGCAAACCCAAGAGCA
GCCTGGAGGAAACCGAGGTGCTGTTCGTGTTCATCGGCTACGACCGGAAG
GCCAGGACCCACAACCCCTACAAGCTGAGCAGCACCCTGACAAACATCTA
CACCGGCAGCAGGCTGCACGAGGCCGGCTGCGCCCCCAGCTACCACGTGG
TCAGGGGCGATATCGCCACCGCCACCGAGGGCGTGATCATCAACGCTGCC
AACAGCAAGGGCCAGCCCGGAGGCGGAGTGTGCGGCGCCCTGTACAAGAA
GTTCCCCGAGAGCTTCGACCTGCAGCCCATCGAGGTGGGCAAGGCCAGGC
TGGTGAAGGGCGCCGCTAAGCACATCATCCACGCCGTGGGCCCCAACTTC
AACAAGGTGAGCGAGGTGGAAGGCGACAAGCAGCTGGCCGAAGCCTACGA
GAGCATCGCCAAGATCGTGAACGACAATAACTACAAGAGCGTGGCCATCC
CACTGCTCAGCACCGGCATCTTCAGCGGCAACAAGGACAGGCTGACCCAG
AGCCTGAACCACCTGCTCACCGCCCTGGACACCACCGATGCCGACGTGGC
CATCTACTGCAGGGACAAGAAGTGGGAGATGACCCTGAAGGAGGCCGTGG
CCAGGCGGGAGGCCGTGGAAGAGATCTGCATCAGCGACGACTCCAGCGTG
ACCGAGCCCGACGCCGAGCTGGTGAGGGTGCACCCCAAGAGCTCCCTGGC
CGGCAGGAAGGGCTACAGCACCAGCGACGGCAAGACCTTCAGCTACCTGG
AGGGCACCAAGTTCCACCAGGCCGCTAAGGACATCGCCGAGATCAACGCT
ATGTGGCCCGTGGCCACCGAGGCCAACGAGCAGGTGTGCATGTACATCCT
GGGCGAGAGCATGTCCAGCATCAGGAGCAAGTGCCCCGTGGAGGAAAGCG
AGGCCAGCACACCACCCAGCACCCTGCCCTGCCTGTGCATCCACGCTATG
ACACCCGAGAGGGTGCAGCGGCTGAAGGCCAGCAGGCCCGAGCAGATCAC
CGTGTGCAGCTCCTTCCCACTGCCCAAGTACAGGATCACCGGCGTGCAGA
AGATCCAGTGCAGCCAGCCCATCCTGTTCAGCCCAAAGGTGCCCGCCTAC
ATCCACCCCAGGAAGTACCTGGTGGAGACCCCACCCGTGGACGAGACACC
CGAGCCAAGCGCCGAGAACCAGAGCACCGAGGGCACACCCGAGCAGCCAC
CCCTGATCACCGAGGACGAGACAAGGACCCGGACCCCAGAGCCCATCATT
ATCGAGGAAGAGGAAGAGGACAGCATCAGCCTGCTGAGCGACGGCCCCAC
CCACCAGGTGCTGCAGGTGGAGGCCGACATCCACGGCCCACCCAGCGTGT
CCAGCTCCAGCTGGAGCATCCCACACGCCAGCGACTTCGACGTGGACAGC
CTGAGCATCCTGGACACCCTGGAGGGCGCCAGCGTGACCTCCGGCGCCAC
CAGCGCCGAGACCAACAGCTACTTCGCCAAGAGCATGGAGTTCCTGGCCA
GGCCCGTGCCAGCTCCCAGGACCGTGTTCAGGAACCCACCCCACCCAGCT
CCCAGGACCAGGACCCCAAGCCTGGCTCCCAGCAGGGCCTGCAGCAGGAC
CAGCCTGGTGAGCACCCCACCCGGCGTGAACAGGGTGATCACCAGGGAGG
AACTGGAGGCCCTGACACCCAGCAGGACCCCCAGCAGGTCCGTGAGCAGG
ACTAGTCTGGTGTCCAACCCACCCGGCGTGAACAGGGTGATCACCAGGGA
GGAATTCGAGGCCTTCGTGGCCCAGCAACAGAGACGGTTCGACGCCGGCG
CCTACATCTTCAGCAGCGACACCGGCCAGGGACACCTGCAGCAAAAGAGC
GTGAGGCAGACCGTGCTGAGCGAGGTGGTGCTGGAGAGGACCGAGCTGGA
AATCAGCTACGCCCCCAGGCTGGACCAGGAGAAGGAGGAACTGCTCAGGA
AGAAACTGCAGCTGAACCCCACCCCAGCCAACAGGAGCAGGTACCAGAGC
AGGAAGGTGGAGAACATGAAGGCCATCACCGCCAGGCGGATCCTGCAGGG
CCTGGGACACTACCTGAAGGCCGAGGGCAAGGTGGAGTGCTACAGGACCC
TGCACCCCGTGCCACTGTACAGCTCCAGCGTGAACAGGGCCTTCTCCAGC
CCCAAGGTGGCCGTGGAGGCCTGCAACGCTATGCTGAAGGAGAACTTCCC
CACCGTGGCCAGCTACTGCATCATCCCCGAGTACGACGCCTACCTGGACA
TGGTGGACGGCGCCAGCTGCTGCCTGGACACCGCCAGCTTCTGCCCCGCC
AAGCTGAGGAGCTTCCCCAAGAAACACAGCTACCTGGAGCCCACCATCAG
GAGCGCCGTGCCCAGCGCCATCCAGAACACCCTGCAGAACGTGCTGGCCG
CTGCCACCAAGAGGAACTGCAACGTGACCCAGATGAGGGAGCTGCCCGTG
CTGGACAGCGCTGCCTTCAACGTGGAGTGCTTCAAGAAATACGCCTGCAA
CAACGAGTACTGGGAGACCTTCAAGGAGAACCCCATCAGGCTGACCGAAG
AGAACGTGGTGAACTACATCACCAAGCTGAAGGGCCCCAAGGCCGCTGCC
CTGTTCGCTAAGACCCACAACCTGAACATGCTGCAGGACATCCCAATGGA
CAGGTTCGTGATGGACCTGAAGAGGGACGTGAAGGTGACACCCGGCACCA
AGCACACCGAGGAGAGGCCCAAGGTGCAGGTGATCCAGGCCGCTGACCCA
CTGGCCACCGCCTACCTGTGCGGCATCCACAGGGAGCTGGTGAGGCGGCT
GAACGCCGTGCTGCTGCCCAACATCCACACCCTGTTCGACATGAGCGCCG
AGGACTTCGACGCCATCATCGCCGAGCACTTCCAGCCCGGCGACTGCGTG
CTGGAGACCGACATCGCCAGCTTCGACAAGAGCGAGGATGACGCTATGGC
CCTGACCGCTCTGATGATCCTGGAGGACCTGGGCGTGGACGCCGAGCTGC
TCACCCTGATCGAGGCTGCCTTCGGCGAGATCAGCTCCATCCACCTGCCC
ACCAAGACCAAGTTCAAGTTCGGCGCTATGATGAAAAGCGGAATGTTCCT
GACCCTGTTCGTGAACACCGTGATCAACATTGTGATCGCCAGCAGGGTGC
TGCGGGAGAGGCTGACCGGCAGCCCCTGCGCTGCCTTCATCGGCGACGAC
AACATCGTGAAGGGCGTGAAAAGCGACAAGCTGATGGCCGACAGGTGCGC
CACCTGGCTGAACATGGAGGTGAAGATCATCGACGCCGTGGTGGGCGAGA
AGGCCCCCTACTTCTGCGGCGGATTCATCCTGTGCGACAGCGTGACCGGC
ACCGCCTGCAGGGTGGCCGACCCCCTGAAGAGGCTGTTCAAGCTGGGCAA
GCCACTGGCCGCTGACGATGAGCACGACGATGACAGGCGGAGGGCCCTGC
ACGAGGAAAGCACCAGGTGGAACAGGGTGGGCATCCTGAGCGAGCTGTGC
AAGGCCGTGGAGAGCAGGTACGAGACCGTGGGCACCAGCATCATCGTGAT
GGCTATGACCACACTGGCCAGCTCCGTCAAGAGCTTCTCCTACCTGAGGG
GGGCCCCTATAACTCTCTACGGCTAACCTGAATGGACTACGACATAGTCT
AGTCCGCCAAGGCCGCCACCACTCGAGTATGTTACGTGCAAAGGTGATTG
TCACCCCCCGAAAGACCATATTGTGACACACCCTCAGTATCACGCCCAAA
CATTTACAGCCGCGGTGTCAAAAACCGCGTGGACGTGGTTAACATCCCTG
CTGGGAGGATCAGCCGTAATTATTATAATTGGCTTGGTGCTGGCTACTAT
TGTGGCCATGTACGTGCTGACCAACCAGAAACATAATTGAATACAGCAGC
AATTGGCAAGCTGCTTACATAGAACTCGCGGCGATTGGCATGCCGCCTTA
AAATTTTTATTTTATTTTTTCTTTTCTTTTCCGAATCGGATTTTGTTTTT
AATATTTCAAAAAAAAAAAAAAAAAAAAAAAAATCTAGAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAA

SEQ ID NO:29 - mARM3326 (mRNA South Africa B.1.351)

aggaaacttaagtcaacacaacatatacaaaacaaacgaatctcaagcaa
tcaagcattctacttctattgcagcaatttaaatcatttcttttaaagca
aaagcaattttctgaaaattttcaccatttacgaacgatagccaccATGT
TCGTGTTCCTGGTGCTGCTGCCCCTGGTGTCTAGCCAGTGCGTGAACCTG
ACCACCAGGACCCAGCTGCCTCCCGCCTACACCAACAGCTTCACCAGGGG
CGTGTACTACCCCGACAAGGTGTTCAGGAGCAGCGTGCTGCACAGCACCC
AGGACCTGTTCCTGCCCTTCTTCAGCAACGTGACCTGGTTCCACGCCATC
CACGTGAGCGGCACCAACGGCACCAAGAGGTTCGcCAACCCCGTGCTGCC
CTTCAACGACGGCGTGTACTTCGCCAGCACCGAGAAGTCCAACATCATCA
GGGGCTGGATCTTCGGCACCACCCTGGACAGCAAGACCCAGAGCCTGCTG
ATCGTGAACAACGCCACCAACGTGGTGATCAAGGTGTGCGAGTTCCAGTT
CTGCAACGACCCCTTCCTGGGCGTGTACTACCACAAGAACAACAAGAGCT
GGATGGAGAGCGAGTTCAGGGTGTACTCCAGCGCCAACAACTGCACCTTC
GAGTACGTGAGCCAGCCCTTCCTGATGGACCTGGAGGGCAAGCAGGGCAA
CTTCAAGAACCTGAGGGAGTTCGTGTTCAAGAACATCGACGGCTACTTCA
AGATCTACAGCAAGCACACCCCTATCAACCTGGTGAGGGgCCTGCCCCAG
GGCTTCAGCGCCCTGGAGCCCCTGGTGGACCTGCCCATCGGCATCAACAT
CACCAGGTTCCAGACCCTGCTGGCCCTGCACAGGAGCTACCTGACCCCTG
GCGACAGCAGCTCCGGCTGGACCGCCGGCGCCGCCGCTTACTACGTGGGC
TACCTGCAGCCCAGGACCTTCCTGCTGAAGTACAACGAGAACGGCACCAT
CACCGACGCCGTGGACTGCGCCCTGGACCCTCTGAGCGAGACAAAGTGCA
CCCTGAAGTCCTTCACCGTGGAGAAGGGCATCTACCAGACCAGCAACTTC
AGGGTGCAGCCCACCGAGAGCATCGTGAGGTTCCCCAACATCACCAACCT
GTGCCCCTTCGGCGAGGTGTTCAACGCCACCAGGTTCGCCAGCGTGTACG
CCTGGAACAGGAAGAGGATCAGCAACTGCGTGGCCGACTACAGCGTGCTG
TATAACAGCGCCAGCTTCAGCACCTTCAAGTGCTACGGCGTGAGCCCCAC
CAAGCTGAACGACCTGTGCTTCACCAACGTGTACGCCGACAGCTTCGTGA
TCAGGGGCGACGAGGTGAGGCAGATCGCCCCTGGCCAGACCGGCAAcATC
GCCGACTACAACTACAAGCTGCCCGACGACTTCACCGGCTGCGTGATCGC
CTGGAACAGCAACAACCTGGACAGCAAGGTGGGCGGCAACTACAACTACC
TGTACCGGCTGTTCAGAAAGAGCAACCTGAAGCCCTTCGAGAGGGACATC
AGCACCGAGATCTACCAGGCCGGCAGCACCCCTTGCAACGGCGTGaAGGG
CTTCAACTGCTACTTCCCTCTGCAGAGCTACGGCTTCCAGCCCACCtACG
GCGTGGGCTACCAGCCCTACAGGGTGGTGGTCCTGAGCTTCGAGCTGCTG
CACGCCCCTGCCACCGTGTGCGGCCCCAAGAAGTCCACCAACCTGGTGAA
GAACAAGTGCGTGAACTTCAACTTCAACGGCCTGACCGGCACCGGCGTGC
TGACCGAGAGCAACAAGAAGTTCCTGCCCTTCCAGCAGTTCGGCAGGGAC
ATCGCCGACACCACCGACGCCGTGAGGGACCCTCAGACCCTGGAGATCCT
GGACATCACCCCTTGCAGCTTCGGCGGCGTGAGCGTGATCACCCCTGGCA
CCAACACCAGCAACCAGGTGGCCGTGCTGTACCAGggcGTGAACTGCACC
GAGGTGCCCGTGGCCATCCACGCCGACCAGCTGACCCCTACCTGGAGGGT
GTACTCCACCGGCAGCAACGTGTTCCAGACCAGGGCCGGCTGCCTGATCG
GCGCCGAGCACGTGAACAACAGCTACGAGTGCGACATCCCCATCGGCGCC
GGCATCTGCGCCAGCTACCAGACCCAGACCAACAGCCCCgGGaGcGCCAG
cAGCGTGGCCAGCCAGAGCATCATCGCCTACACCATGAGCCTGGGCGtgG
AGAACAGCGTGGCCTACAGCAACAACAGCATCGCCATCCCCACCAACTTC
ACCATCAGCGTGACCACCGAGATCCTGCCCGTGAGCATGACCAAGACCAG
CGTGGACTGCACCATGTATATCTGCGGCGACAGCACCGAGTGCAGCAACC
TGCTGCTCCAGTACGGCAGCTTCTGCACCCAGCTGAACAGGGCCCTGACC
GGCATCGCCGTGGAGCAGGACAAGAACACCCAGGAGGTGTTCGCCCAGGT
GAAGCAGATCTACAAGACCCCTCCCATCAAGGACTTCGGCGGCTTCAACT
TCAGCCAGATCCTGCCCGACCCCAGCAAGCCCAGCAAGAGGAGCTTCATC
GAGGACCTGCTGTTCAACAAGGTGACCCTGGCCGACGCCGGCTTCATCAA
GCAGTACGGCGACTGCCTGGGCGACATCGCCGCCAGGGACCTGATCTGCG
CCCAGAAGTTCAACGGCCTGACCGTGCTGCCTCCCCTGCTGACCGACGAG
ATGATCGCCCAGTACACCAGCGCCCTGCTGGCCGGCACCATCACCAGCGG
CTGGACCTTCGGCGCCGGCGCCGCCCTGCAGATCCCCTTCGCCATGCAGA
TGGCCTACAGGTTCAACGGCATCGGCGTGACCCAGAACGTGCTGTACGAG
AACCAGAAGCTGATCGCCAACCAGTTCAACAGCGCCATCGGCAAGATCCA
GGACAGCCTGAGCAGCACCGCCAGCGCCCTGGGCAAGCTGCAGGACGTGG
TGAACCAGAACGCCCAGGCCCTGAACACCCTGGTGAAGCAGCTGAGCAGC
AACTTCGGCGCCATCAGCAGCGTGCTGAACGACATCCTGAGCAGGCTGGA
CccacccGAGGCCGAGGTGCAGATCGACAGGCTGATCACCGGCAGGCTGC
AGAGCCTGCAGACCTACGTGACCCAGCAGCTGATCAGGGCCGCCGAGATC
AGGGCCAGCGCCAACCTGGCCGCCACCAAGATGAGCGAGTGCGTGCTGGG
CCAGAGCAAGAGGGTGGACTTCTGCGGCAAGGGCTACCACCTGATGAGCT
TCCCTCAGAGCGCCCCTCACGGCGTGGTGTTCCTGCACGTGACCTACGTG
CCCGCCCAGGAGAAGAACTTCACCACAGCCCCTGCCATCTGCCACGACGG
CAAGGCCCACTTCCCCAGGGAGGGCGTGTTCGTGAGCAACGGCACCCACT
GGTTCGTGACCCAGAGGAACTTCTACGAGCCCCAGATCATCACCACCGAC
AACACCTTCGTGAGCGGCAACTGCGACGTGGTGATCGGCATCGTGAACAA
CACCGTGTACGACCCTCTGCAGCCCGAGCTGGACAGCTTCAAGGAGGAGC
TGGACAAGTACTTCAAGAACCACACCAGCCCCGACGTGGACCTGGGCGAC
ATCAGCGGCATCAACGCCAGCGTGGTGAACATCCAGAAGGAGATCGACAG
GCTGAACGAGGTGGCCAAGAACCTGAACGAGAGCCTGATCGACCTGCAGG
AGCTGGGCAAGTACGAGCAGTACATCAAGTGGCCCTGGTACATCTGGCTG
GGCTTCATCGCCGGCCTGATCGCCATCGTGATGGTGACCATCATGCTGTG
CTGCATGACCAGCTGCTGCAGCTGCCTGAAGGGCTGCTGCAGCTGCGGCA
GCTGCTGCAAGTTCGACGAGGACGACAGCGAGCCCGTGCTGAAGGGCGTG
AAGCTGCACTACACCTaAactcgagctagtgactgactaggatctggtta
ccactaaaccagcctcaagaacacccgaatggagtctctaagctacataa
taccaacttacacttacaaaatgttgtcccccaaaatgtagccattcgta
tctgctcctaataaaaagaaagtttcttcacattctagAAAAAAAAAAAA
AAAAAAAAAAAAAAAAAAAAAAAAAAAAAAAAAAAAAAAAAAAAAAAAAA
AAAAAAAAAAAAAAAAAAaaaaaaaaaaaaaaaaaaaa

SEQ ID NO:30 - Transgene (nucleic acid sequence; mARM3326/SEQ ID NO:29)

ATGTTCGTGTTCCTGGTGCTGCTGCCCCTGGTGTCTAGCCAGTGCGTGAA
CCTGACCACCAGGACCCAGCTGCCTCCCGCCTACACCAACAGCTTCACCA
GGGGCGTGTACTACCCCGACAAGGTGTTCAGGAGCAGCGTGCTGCACAGC
ACCCAGGACCTGTTCCTGCCCTTCTTCAGCAACGTGACCTGGTTCCACGC
CATCCACGTGAGCGGCACCAACGGCACCAAGAGGTTCGcCAACCCCGTGC
TGCCCTTCAACGACGGCGTGTACTTCGCCAGCACCGAGAAGTCCAACATC
ATCAGGGGCTGGATCTTCGGCACCACCCTGGACAGCAAGACCCAGAGCCT
GCTGATCGTGAACAACGCCACCAACGTGGTGATCAAGGTGTGCGAGTTCC
AGTTCTGCAACGACCCCTTCCTGGGCGTGTACTACCACAAGAACAACAAG
AGCTGGATGGAGAGCGAGTTCAGGGTGTACTCCAGCGCCAACAACTGCAC
CTTCGAGTACGTGAGCCAGCCCTTCCTGATGGACCTGGAGGGCAAGCAGG
GCAACTTCAAGAACCTGAGGGAGTTCGTGTTCAAGAACATCGACGGCTAC
TTCAAGATCTACAGCAAGCACACCCCTATCAACCTGGTGAGGGgCCTGCC
CCAGGGCTTCAGCGCCCTGGAGCCCCTGGTGGACCTGCCCATCGGCATCA
ACATCACCAGGTTCCAGACCCTGCTGGCCCTGCACAGGAGCTACCTGACC
CCTGGCGACAGCAGCTCCGGCTGGACCGCCGGCGCCGCCGCTTACTACGT
GGGCTACCTGCAGCCCAGGACCTTCCTGCTGAAGTACAACGAGAACGGCA
CCATCACCGACGCCGTGGACTGCGCCCTGGACCCTCTGAGCGAGACAAAG
TGCACCCTGAAGTCCTTCACCGTGGAGAAGGGCATCTACCAGACCAGCAA
CTTCAGGGTGCAGCCCACCGAGAGCATCGTGAGGTTCCCCAACATCACCA
ACCTGTGCCCCTTCGGCGAGGTGTTCAACGCCACCAGGTTCGCCAGCGTG
TACGCCTGGAACAGGAAGAGGATCAGCAACTGCGTGGCCGACTACAGCGT
GCTGTATAACAGCGCCAGCTTCAGCACCTTCAAGTGCTACGGCGTGAGCC
CCACCAAGCTGAACGACCTGTGCTTCACCAACGTGTACGCCGACAGCTTC
GTGATCAGGGGCGACGAGGTGAGGCAGATCGCCCCTGGCCAGACCGGCAA
cATCGCCGACTACAACTACAAGCTGCCCGACGACTTCACCGGCTGCGTGA
TCGCCTGGAACAGCAACAACCTGGACAGCAAGGTGGGCGGCAACTACAAC
TACCTGTACCGGCTGTTCAGAAAGAGCAACCTGAAGCCCTTCGAGAGGGA
CATCAGCACCGAGATCTACCAGGCCGGCAGCACCCCTTGCAACGGCGTGa
AGGGCTTCAACTGCTACTTCCCTCTGCAGAGCTACGGCTTCCAGCCCACC
tACGGCGTGGGCTACCAGCCCTACAGGGTGGTGGTCCTGAGCTTCGAGCT
GCTGCACGCCCCTGCCACCGTGTGCGGCCCCAAGAAGTCCACCAACCTGG
TGAAGAACAAGTGCGTGAACTTCAACTTCAACGGCCTGACCGGCACCGGC
GTGCTGACCGAGAGCAACAAGAAGTTCCTGCCCTTCCAGCAGTTCGGCAG
GGACATCGCCGACACCACCGACGCCGTGAGGGACCCTCAGACCCTGGAGA
TCCTGGACATCACCCCTTGCAGCTTCGGCGGCGTGAGCGTGATCACCCCT
GGCACCAACACCAGCAACCAGGTGGCCGTGCTGTACCAGggcGTGAACTG
CACCGAGGTGCCCGTGGCCATCCACGCCGACCAGCTGACCCCTACCTGGA
GGGTGTACTCCACCGGCAGCAACGTGTTCCAGACCAGGGCCGGCTGCCTG
ATCGGCGCCGAGCACGTGAACAACAGCTACGAGTGCGACATCCCCATCGG
CGCCGGCATCTGCGCCAGCTACCAGACCCAGACCAACAGCCCCgGGaGcG
CCAGcAGCGTGGCCAGCCAGAGCATCATCGCCTACACCATGAGCCTGGGC
GtgGAGAACAGCGTGGCCTACAGCAACAACAGCATCGCCATCCCCACCAA
CTTCACCATCAGCGTGACCACCGAGATCCTGCCCGTGAGCATGACCAAGA
CCAGCGTGGACTGCACCATGTATATCTGCGGCGACAGCACCGAGTGCAGC
AACCTGCTGCTCCAGTACGGCAGCTTCTGCACCCAGCTGAACAGGGCCCT
GACCGGCATCGCCGTGGAGCAGGACAAGAACACCCAGGAGGTGTTCGCCC
AGGTGAAGCAGATCTACAAGACCCCTCCCATCAAGGACTTCGGCGGCTTC
AACTTCAGCCAGATCCTGCCCGACCCCAGCAAGCCCAGCAAGAGGAGCTT
CATCGAGGACCTGCTGTTCAACAAGGTGACCCTGGCCGACGCCGGCTTCA
TCAAGCAGTACGGCGACTGCCTGGGCGACATCGCCGCCAGGGACCTGATC
TGCGCCCAGAAGTTCAACGGCCTGACCGTGCTGCCTCCCCTGCTGACCGA
CGAGATGATCGCCCAGTACACCAGCGCCCTGCTGGCCGGCACCATCACCA
GCGGCTGGACCTTCGGCGCCGGCGCCGCCCTGCAGATCCCCTTCGCCATG
CAGATGGCCTACAGGTTCAACGGCATCGGCGTGACCCAGAACGTGCTGTA
CGAGAACCAGAAGCTGATCGCCAACCAGTTCAACAGCGCCATCGGCAAGA
TCCAGGACAGCCTGAGCAGCACCGCCAGCGCCCTGGGCAAGCTGCAGGAC
GTGGTGAACCAGAACGCCCAGGCCCTGAACACCCTGGTGAAGCAGCTGAG
CAGCAACTTCGGCGCCATCAGCAGCGTGCTGAACGACATCCTGAGCAGGC
TGGACccacccGAGGCCGAGGTGCAGATCGACAGGCTGATCACCGGCAGG
CTGCAGAGCCTGCAGACCTACGTGACCCAGCAGCTGATCAGGGCCGCCGA
GATCAGGGCCAGCGCCAACCTGGCCGCCACCAAGATGAGCGAGTGCGTGC
TGGGCCAGAGCAAGAGGGTGGACTTCTGCGGCAAGGGCTACCACCTGATG
AGCTTCCCTCAGAGCGCCCCTCACGGCGTGGTGTTCCTGCACGTGACCTA
CGTGCCCGCCCAGGAGAAGAACTTCACCACAGCCCCTGCCATCTGCCACG
ACGGCAAGGCCCACTTCCCCAGGGAGGGCGTGTTCGTGAGCAACGGCACC
CACTGGTTCGTGACCCAGAGGAACTTCTACGAGCCCCAGATCATCACCAC
CGACAACACCTTCGTGAGCGGCAACTGCGACGTGGTGATCGGCATCGTGA
ACAACACCGTGTACGACCCTCTGCAGCCCGAGCTGGACAGCTTCAAGGAG
GAGCTGGACAAGTACTTCAAGAACCACACCAGCCCCGACGTGGACCTGGG
CGACATCAGCGGCATCAACGCCAGCGTGGTGAACATCCAGAAGGAGATCG
ACAGGCTGAACGAGGTGGCCAAGAACCTGAACGAGAGCCTGATCGACCTG
CAGGAGCTGGGCAAGTACGAGCAGTACATCAAGTGGCCCTGGTACATCTG
GCTGGGCTTCATCGCCGGCCTGATCGCCATCGTGATGGTGACCATCATGC
TGTGCTGCATGACCAGCTGCTGCAGCTGCCTGAAGGGCTGCTGCAGCTGC
GGCAGCTGCTGCAAGTTCGACGAGGACGACAGCGAGCCCGTGCTGAAGGG
CGTGAAGCTGCACTACACCTaA

SEQ ID NO:31 - Transgene (amino acid sequence; mARM3326/SEQ ID NO:29)

MFVFLVLLPLVSSQCVNLTTRTQLPPAYTNSFTRGVYYPDKVFRSSVLHS
TQDLFLPFFSNVTWFHAIHVSGTNGTKRFANPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRGLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGNIADYNYKLPDDFTGCVIAWNSNNLDSKVGGNYN
YLYRLFRKSNLKPFERDISTEIYQAGSTPCNGVKGFNCYFPLQSYGFQPT
YGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQGVNCTEVPVAIHADQLTPTWRVYSTGSNVFQTRAGCL
IGAEHVNNSYECDIPIGAGICASYQTQTNSPGSASSVASQSIIAYTMSLG
VENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT*

SEQ ID NO:32 - mARM3290 (mRNA, D614G)

aggaaacttaagtcaacacaacatatacaaaacaaacgaatctcaagcaa
tcaagcattctacttctattgcagcaatttaaatcatttcttttaaagca
aaagcaattttctgaaaattttcaccatttacgaacgatagccaccATGT
TCGTGTTCCTGGTGCTGCTGCCCCTGGTGTCTAGCCAGTGCGTGAACCTG
ACCACCAGGACCCAGCTGCCTCCCGCCTACACCAACAGCTTCACCAGGGG
CGTGTACTACCCCGACAAGGTGTTCAGGAGCAGCGTGCTGCACAGCACCC
AGGACCTGTTCCTGCCCTTCTTCAGCAACGTGACCTGGTTCCACGCCATC
CACGTGAGCGGCACCAACGGCACCAAGAGGTTCGACAACCCCGTGCTGCC
CTTCAACGACGGCGTGTACTTCGCCAGCACCGAGAAGTCCAACATCATCA
GGGGCTGGATCTTCGGCACCACCCTGGACAGCAAGACCCAGAGCCTGCTG
ATCGTGAACAACGCCACCAACGTGGTGATCAAGGTGTGCGAGTTCCAGTT
CTGCAACGACCCCTTCCTGGGCGTGTACTACCACAAGAACAACAAGAGCT
GGATGGAGAGCGAGTTCAGGGTGTACTCCAGCGCCAACAACTGCACCTTC
GAGTACGTGAGCCAGCCCTTCCTGATGGACCTGGAGGGCAAGCAGGGCAA
CTTCAAGAACCTGAGGGAGTTCGTGTTCAAGAACATCGACGGCTACTTCA
AGATCTACAGCAAGCACACCCCTATCAACCTGGTGAGGGACCTGCCCCAG
GGCTTCAGCGCCCTGGAGCCCCTGGTGGACCTGCCCATCGGCATCAACAT
CACCAGGTTCCAGACCCTGCTGGCCCTGCACAGGAGCTACCTGACCCCTG
GCGACAGCAGCTCCGGCTGGACCGCCGGCGCCGCCGCTTACTACGTGGGC
TACCTGCAGCCCAGGACCTTCCTGCTGAAGTACAACGAGAACGGCACCAT
CACCGACGCCGTGGACTGCGCCCTGGACCCTCTGAGCGAGACAAAGTGCA
CCCTGAAGTCCTTCACCGTGGAGAAGGGCATCTACCAGACCAGCAACTTC
AGGGTGCAGCCCACCGAGAGCATCGTGAGGTTCCCCAACATCACCAACCT
GTGCCCCTTCGGCGAGGTGTTCAACGCCACCAGGTTCGCCAGCGTGTACG
CCTGGAACAGGAAGAGGATCAGCAACTGCGTGGCCGACTACAGCGTGCTG
TATAACAGCGCCAGCTTCAGCACCTTCAAGTGCTACGGCGTGAGCCCCAC
CAAGCTGAACGACCTGTGCTTCACCAACGTGTACGCCGACAGCTTCGTGA
TCAGGGGCGACGAGGTGAGGCAGATCGCCCCTGGCCAGACCGGCAAGATC
GCCGACTACAACTACAAGCTGCCCGACGACTTCACCGGCTGCGTGATCGC
CTGGAACAGCAACAACCTGGACAGCAAGGTGGGCGGCAACTACAACTACC
TGTACCGGCTGTTCAGAAAGAGCAACCTGAAGCCCTTCGAGAGGGACATC
AGCACCGAGATCTACCAGGCCGGCAGCACCCCTTGCAACGGCGTGGAGGG
CTTCAACTGCTACTTCCCTCTGCAGAGCTACGGCTTCCAGCCCACCAACG
GCGTGGGCTACCAGCCCTACAGGGTGGTGGTCCTGAGCTTCGAGCTGCTG
CACGCCCCTGCCACCGTGTGCGGCCCCAAGAAGTCCACCAACCTGGTGAA
GAACAAGTGCGTGAACTTCAACTTCAACGGCCTGACCGGCACCGGCGTGC
TGACCGAGAGCAACAAGAAGTTCCTGCCCTTCCAGCAGTTCGGCAGGGAC
ATCGCCGACACCACCGACGCCGTGAGGGACCCTCAGACCCTGGAGATCCT
GGACATCACCCCTTGCAGCTTCGGCGGCGTGAGCGTGATCACCCCTGGCA
CCAACACCAGCAACCAGGTGGCCGTGCTGTACCAGggcGTGAACTGCACC
GAGGTGCCCGTGGCCATCCACGCCGACCAGCTGACCCCTACCTGGAGGGT
GTACTCCACCGGCAGCAACGTGTTCCAGACCAGGGCCGGCTGCCTGATCG
GCGCCGAGCACGTGAACAACAGCTACGAGTGCGACATCCCCATCGGCGCC
GGCATCTGCGCCAGCTACCAGACCCAGACCAACAGCCCCgGGaGcGCCAG
cAGCGTGGCCAGCCAGAGCATCATCGCCTACACCATGAGCCTGGGCGCCG
AGAACAGCGTGGCCTACAGCAACAACAGCATCGCCATCCCCACCAACTTC
ACCATCAGCGTGACCACCGAGATCCTGCCCGTGAGCATGACCAAGACCAG
CGTGGACTGCACCATGTATATCTGCGGCGACAGCACCGAGTGCAGCAACC
TGCTGCTCCAGTACGGCAGCTTCTGCACCCAGCTGAACAGGGCCCTGACC
GGCATCGCCGTGGAGCAGGACAAGAACACCCAGGAGGTGTTCGCCCAGGT
GAAGCAGATCTACAAGACCCCTCCCATCAAGGACTTCGGCGGCTTCAACT
TCAGCCAGATCCTGCCCGACCCCAGCAAGCCCAGCAAGAGGAGCTTCATC
GAGGACCTGCTGTTCAACAAGGTGACCCTGGCCGACGCCGGCTTCATCAA
GCAGTACGGCGACTGCCTGGGCGACATCGCCGCCAGGGACCTGATCTGCG
CCCAGAAGTTCAACGGCCTGACCGTGCTGCCTCCCCTGCTGACCGACGAG
ATGATCGCCCAGTACACCAGCGCCCTGCTGGCCGGCACCATCACCAGCGG
CTGGACCTTCGGCGCCGGCGCCGCCCTGCAGATCCCCTTCGCCATGCAGA
TGGCCTACAGGTTCAACGGCATCGGCGTGACCCAGAACGTGCTGTACGAG
AACCAGAAGCTGATCGCCAACCAGTTCAACAGCGCCATCGGCAAGATCCA
GGACAGCCTGAGCAGCACCGCCAGCGCCCTGGGCAAGCTGCAGGACGTGG
TGAACCAGAACGCCCAGGCCCTGAACACCCTGGTGAAGCAGCTGAGCAGC
AACTTCGGCGCCATCAGCAGCGTGCTGAACGACATCCTGAGCAGGCTGGA
CccacccGAGGCCGAGGTGCAGATCGACAGGCTGATCACCGGCAGGCTGC
AGAGCCTGCAGACCTACGTGACCCAGCAGCTGATCAGGGCCGCCGAGATC
AGGGCCAGCGCCAACCTGGCCGCCACCAAGATGAGCGAGTGCGTGCTGGG
CCAGAGCAAGAGGGTGGACTTCTGCGGCAAGGGCTACCACCTGATGAGCT
TCCCTCAGAGCGCCCCTCACGGCGTGGTGTTCCTGCACGTGACCTACGTG
CCCGCCCAGGAGAAGAACTTCACCACAGCCCCTGCCATCTGCCACGACGG
CAAGGCCCACTTCCCCAGGGAGGGCGTGTTCGTGAGCAACGGCACCCACT
GGTTCGTGACCCAGAGGAACTTCTACGAGCCCCAGATCATCACCACCGAC
AACACCTTCGTGAGCGGCAACTGCGACGTGGTGATCGGCATCGTGAACAA
CACCGTGTACGACCCTCTGCAGCCCGAGCTGGACAGCTTCAAGGAGGAGC
TGGACAAGTACTTCAAGAACCACACCAGCCCCGACGTGGACCTGGGCGAC
ATCAGCGGCATCAACGCCAGCGTGGTGAACATCCAGAAGGAGATCGACAG
GCTGAACGAGGTGGCCAAGAACCTGAACGAGAGCCTGATCGACCTGCAGG
AGCTGGGCAAGTACGAGCAGTACATCAAGTGGCCCTGGTACATCTGGCTG
GGCTTCATCGCCGGCCTGATCGCCATCGTGATGGTGACCATCATGCTGTG
CTGCATGACCAGCTGCTGCAGCTGCCTGAAGGGCTGCTGCAGCTGCGGCA
GCTGCTGCAAGTTCGACGAGGACGACAGCGAGCCCGTGCTGAAGGGCGTG
AAGCTGCACTACACCTaAactcgagctagtgactgactaggatctggtta
ccactaaaccagcctcaagaacacccgaatggagtctctaagctacataa
taccaacttacacttacaaaatgttgtcccccaaaatgtagccattcgta
tctgctcctaataaaaagaaagtttcttcacattctagAAAAAAAAAAAA
AAAAAAAAAAAAAAAAAAAAAAAAAAAAAAAAAAAAAAAAAAAAAAAAAA
AAAAAAAAAAAAAAAAAAaaaaaaaaaaaaaaaaaaaa

SEQ ID NO:33 - Transgene (nucleic acid sequence; mARM3290/SEQ ID NO:32)

ATGTTCGTGTTCCTGGTGCTGCTGCCCCTGGTGTCTAGCCAGTGCGTGAA
CCTGACCACCAGGACCCAGCTGCCTCCCGCCTACACCAACAGCTTCACCA
GGGGCGTGTACTACCCCGACAAGGTGTTCAGGAGCAGCGTGCTGCACAGC
ACCCAGGACCTGTTCCTGCCCTTCTTCAGCAACGTGACCTGGTTCCACGC
CATCCACGTGAGCGGCACCAACGGCACCAAGAGGTTCGACAACCCCGTGC
TGCCCTTCAACGACGGCGTGTACTTCGCCAGCACCGAGAAGTCCAACATC
ATCAGGGGCTGGATCTTCGGCACCACCCTGGACAGCAAGACCCAGAGCCT
GCTGATCGTGAACAACGCCACCAACGTGGTGATCAAGGTGTGCGAGTTCC
AGTTCTGCAACGACCCCTTCCTGGGCGTGTACTACCACAAGAACAACAAG
AGCTGGATGGAGAGCGAGTTCAGGGTGTACTCCAGCGCCAACAACTGCAC
CTTCGAGTACGTGAGCCAGCCCTTCCTGATGGACCTGGAGGGCAAGCAGG
GCAACTTCAAGAACCTGAGGGAGTTCGTGTTCAAGAACATCGACGGCTAC
TTCAAGATCTACAGCAAGCACACCCCTATCAACCTGGTGAGGGACCTGCC
CCAGGGCTTCAGCGCCCTGGAGCCCCTGGTGGACCTGCCCATCGGCATCA
ACATCACCAGGTTCCAGACCCTGCTGGCCCTGCACAGGAGCTACCTGACC
CCTGGCGACAGCAGCTCCGGCTGGACCGCCGGCGCCGCCGCTTACTACGT
GGGCTACCTGCAGCCCAGGACCTTCCTGCTGAAGTACAACGAGAACGGCA
CCATCACCGACGCCGTGGACTGCGCCCTGGACCCTCTGAGCGAGACAAAG
TGCACCCTGAAGTCCTTCACCGTGGAGAAGGGCATCTACCAGACCAGCAA
CTTCAGGGTGCAGCCCACCGAGAGCATCGTGAGGTTCCCCAACATCACCA
ACCTGTGCCCCTTCGGCGAGGTGTTCAACGCCACCAGGTTCGCCAGCGTG
TACGCCTGGAACAGGAAGAGGATCAGCAACTGCGTGGCCGACTACAGCGT
GCTGTATAACAGCGCCAGCTTCAGCACCTTCAAGTGCTACGGCGTGAGCC
CCACCAAGCTGAACGACCTGTGCTTCACCAACGTGTACGCCGACAGCTTC
GTGATCAGGGGCGACGAGGTGAGGCAGATCGCCCCTGGCCAGACCGGCAA
GATCGCCGACTACAACTACAAGCTGCCCGACGACTTCACCGGCTGCGTGA
TCGCCTGGAACAGCAACAACCTGGACAGCAAGGTGGGCGGCAACTACAAC
TACCTGTACCGGCTGTTCAGAAAGAGCAACCTGAAGCCCTTCGAGAGGGA
CATCAGCACCGAGATCTACCAGGCCGGCAGCACCCCTTGCAACGGCGTGG
AGGGCTTCAACTGCTACTTCCCTCTGCAGAGCTACGGCTTCCAGCCCACC
AACGGCGTGGGCTACCAGCCCTACAGGGTGGTGGTCCTGAGCTTCGAGCT
GCTGCACGCCCCTGCCACCGTGTGCGGCCCCAAGAAGTCCACCAACCTGG
TGAAGAACAAGTGCGTGAACTTCAACTTCAACGGCCTGACCGGCACCGGC
GTGCTGACCGAGAGCAACAAGAAGTTCCTGCCCTTCCAGCAGTTCGGCAG
GGACATCGCCGACACCACCGACGCCGTGAGGGACCCTCAGACCCTGGAGA
TCCTGGACATCACCCCTTGCAGCTTCGGCGGCGTGAGCGTGATCACCCCT
GGCACCAACACCAGCAACCAGGTGGCCGTGCTGTACCAGggcGTGAACTG
CACCGAGGTGCCCGTGGCCATCCACGCCGACCAGCTGACCCCTACCTGGA
GGGTGTACTCCACCGGCAGCAACGTGTTCCAGACCAGGGCCGGCTGCCTG
ATCGGCGCCGAGCACGTGAACAACAGCTACGAGTGCGACATCCCCATCGG
CGCCGGCATCTGCGCCAGCTACCAGACCCAGACCAACAGCCCCgGGaGcG
CCAGcAGCGTGGCCAGCCAGAGCATCATCGCCTACACCATGAGCCTGGGC
GCCGAGAACAGCGTGGCCTACAGCAACAACAGCATCGCCATCCCCACCAA
CTTCACCATCAGCGTGACCACCGAGATCCTGCCCGTGAGCATGACCAAGA
CCAGCGTGGACTGCACCATGTATATCTGCGGCGACAGCACCGAGTGCAGC
AACCTGCTGCTCCAGTACGGCAGCTTCTGCACCCAGCTGAACAGGGCCCT
GACCGGCATCGCCGTGGAGCAGGACAAGAACACCCAGGAGGTGTTCGCCC
AGGTGAAGCAGATCTACAAGACCCCTCCCATCAAGGACTTCGGCGGCTTC
AACTTCAGCCAGATCCTGCCCGACCCCAGCAAGCCCAGCAAGAGGAGCTT
CATCGAGGACCTGCTGTTCAACAAGGTGACCCTGGCCGACGCCGGCTTCA
TCAAGCAGTACGGCGACTGCCTGGGCGACATCGCCGCCAGGGACCTGATC
TGCGCCCAGAAGTTCAACGGCCTGACCGTGCTGCCTCCCCTGCTGACCGA
CGAGATGATCGCCCAGTACACCAGCGCCCTGCTGGCCGGCACCATCACCA
GCGGCTGGACCTTCGGCGCCGGCGCCGCCCTGCAGATCCCCTTCGCCATG
CAGATGGCCTACAGGTTCAACGGCATCGGCGTGACCCAGAACGTGCTGTA
CGAGAACCAGAAGCTGATCGCCAACCAGTTCAACAGCGCCATCGGCAAGA
TCCAGGACAGCCTGAGCAGCACCGCCAGCGCCCTGGGCAAGCTGCAGGAC
GTGGTGAACCAGAACGCCCAGGCCCTGAACACCCTGGTGAAGCAGCTGAG
CAGCAACTTCGGCGCCATCAGCAGCGTGCTGAACGACATCCTGAGCAGGC
TGGACccacccGAGGCCGAGGTGCAGATCGACAGGCTGATCACCGGCAGG
CTGCAGAGCCTGCAGACCTACGTGACCCAGCAGCTGATCAGGGCCGCCGA
GATCAGGGCCAGCGCCAACCTGGCCGCCACCAAGATGAGCGAGTGCGTGC
TGGGCCAGAGCAAGAGGGTGGACTTCTGCGGCAAGGGCTACCACCTGATG
AGCTTCCCTCAGAGCGCCCCTCACGGCGTGGTGTTCCTGCACGTGACCTA
CGTGCCCGCCCAGGAGAAGAACTTCACCACAGCCCCTGCCATCTGCCACG
ACGGCAAGGCCCACTTCCCCAGGGAGGGCGTGTTCGTGAGCAACGGCACC
CACTGGTTCGTGACCCAGAGGAACTTCTACGAGCCCCAGATCATCACCAC
CGACAACACCTTCGTGAGCGGCAACTGCGACGTGGTGATCGGCATCGTGA
ACAACACCGTGTACGACCCTCTGCAGCCCGAGCTGGACAGCTTCAAGGAG
GAGCTGGACAAGTACTTCAAGAACCACACCAGCCCCGACGTGGACCTGGG
CGACATCAGCGGCATCAACGCCAGCGTGGTGAACATCCAGAAGGAGATCG
ACAGGCTGAACGAGGTGGCCAAGAACCTGAACGAGAGCCTGATCGACCTG
CAGGAGCTGGGCAAGTACGAGCAGTACATCAAGTGGCCCTGGTACATCTG
GCTGGGCTTCATCGCCGGCCTGATCGCCATCGTGATGGTGACCATCATGC
TGTGCTGCATGACCAGCTGCTGCAGCTGCCTGAAGGGCTGCTGCAGCTGC
GGCAGCTGCTGCAAGTTCGACGAGGACGACAGCGAGCCCGTGCTGAAGGG
CGTGAAGCTGCACTACACCTaA

SEQ ID NO:34 - Transgene (amino acid sequence; mARM3290/SEQ ID NO:32)

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQGVNCTEVPVAIHADQLTPTWRVYSTGSNVFQTRAGCL
IGAEHVNNSYECDIPIGAGICASYQTQTNSPGSASSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT*

SEQ ID NO:35 - 5′ UTR (TEV)

AGGAAACTTAAGTCAACACAACATATACAAAACAAACGAATCTCAAGCAA
TCAAGCATTCTACTTCTATTGCAGCAATTTAAATCATTTCTTTTAAAGCA
AAAGCAATTTTCTGAAAATTTTCACCATTTACGAACGATAGCCACC

SEQ ID NO:36 - 3′ UTR (Xbg), with poly-A

ACTCGAGCTAGTGACTGACTAGGATCTGGTTACCACTAAACCAGCCTCAA
GAACACCCGAATGGAGTCTCTAAGCTACATAATACCAACTTACACTTACA
AAATGTTGTCCCCCAAAATGTAGCCATTCGTATCTGCTCCTAATAAAAAG
AAAGTTTCTTCACATTCTAGAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAA

SEQ ID NO:37 - 3′ UTR (Xbg), without poly-A

ACTCGAGCTAGTGACTGACTAGGATCTGGTTACCACTAAACCAGCCTCAA
GAACACCCGAATGGAGTCTCTAAGCTACATAATACCAACTTACACTTACA
AAATGTTGTCCCCCAAAATGTAGCCATTCGTATCTGCTCCTAATAAAAAG
AAAGTTTCTTCACATTCTAG

SEQ ID NO:38 - 5′ UTR (alternative VEEV-derived sequence)

GATGGGCGGCGCATGAGAGAAGCCCAGACCAATTACCTACCCAAA

SEQ ID NO:39 - 5′ UTR (alternative VEEV-derived sequence)

GATAGGCGGCGCATGAGAGAAGCCCAGACCAATTACCTACCCAAA

SEQ ID NO:40 - mARM3124

atgggcggcgcatgagagaagcccagaccaattacctacccaaaatggag
aaagttcacgttgacatcgaggaagacagcccattcctcagagctttgca
gcggagcttcccgcagtttgaggtagaagccaagcaggtcactgataatg
accatgctaatgccagagcgttttcgcatctggcttcaaaactgatcgaa
acggaggtggacccatccgacacgatccttgacattggaagtgcgcccgc
ccgcagaatGTATTCTAAGCACAAGTATCATTGTATCtgtccgatgagat
gtgcggaagatccggacagattgtataagtatgcaactaagctgaagaaa
aactgtaaggaaataactgataaggaattggacaagaaaatgaaggagct
ggccgccgtcatgagcgaccctgacctggaaactgagactatgtgcctcc
acgacgacgagtcgtgtcgctacgaagggcaagtcgctgtttaccaggat
gtatacgcCGTcGACGGCCCCACCAGCCTGTACCACCAGGCCAACAAGGG
CGTGAGGGTGGCCTACTGGATCGGCTTCGACACCACACCCTTCATGTTCA
AGAACCTGGCCGGCGCCTACCCCAGCTACAGCACCAACTGGGCCGACGAG
ACAGTGCTGACCGCCAGGAACATCGGCCTGTGCAGCAGCGACGTGATGGA
GAGGAGCCGGAGGGGCATGAGCATCCTGAGGAAGAAGTACCTGAAGCCCA
GCAACAACGTGCTGTTCAGCGTGGGCAGCACCATCTACCACGAGAAGAGG
GACCTGCTGAGGAGCTGGCACCTGCCCAGCGTGTTCCACCTGAGGGGCAA
GCAGAACTACACCTGCAGGTGCGAGACAATCGTGAGCTGCGACGGCTACG
TGGTGAAGAGGATCGCCATCAGCCCCGGCCTGTACGGCAAGCCCAGCGGC
TACGCCGCCACCATGCACAGGGAGGGCTTCCTGTGCTGCAAGGTGACCGA
CACCCTGAACGGCGAGAGGGTGAGCTTCCCCGTGTGCACCTACGTGCCCG
CCACCCTGTGCGACCAGATGACCGGCATCCTGGCCACCGACGTGAGCGCC
GACGACGCCCAGAAGCTGCTGGTGGGCCTGAACCAGAGGATCGTGGTGAA
CGGCAGGACCCAGAGGAACACCAACACCATGAAGAACTACCTGCTGCCCG
TGGTGGCCCAGGCCTTCGCCAGGTGGGCCAAGGAGTACAAGGAGGACCAG
GAGGACGAGAGGCCCCTGGGCCTGAGGGACcGaCAGCTGGTGATGGGCTG
CTGCTGGGCCTTCAGGCGGCACAAGATCACCAGCATCTACAAGAGGCCCG
ACACCCAGACCATCATCAAGGTGAACAGCGACTTCCACAGCTTCGTGCTG
CCCAGGATCGGCAGCAACACCCTGGAGATCGGCCTGAGGACCCGGATCAG
GAAGATGCTGGAGGAGCACAAGGAGCCCAGCCCTCTGATCACCGCCGAGG
ACGTGCAGGAGGCCAAGTGCGCCGCCGACGAGGCCAAGGAGGTGAGGGAG
GCCGAGGAGCTGAGGGCCGCCCTGCCTCCCCTGGCCGCCGACGTGGAGGA
GCCCACCCTGGAGGCCGACGTGGACCTGATGCTGCAGGAGGCCGGCGCCG
GCAGCGTGGAGACACCCAGGGGCCTGATCAAGGTGACCAGCTACGACGGC
GAGGACAAGATCGGCAGCTACGCCGTGCTcAGCCCTCAGGCCGTGCTGAA
GTCCGAGAAGCTGAGCTGCATCCACCCTCTGGCCGAGCAGGTGATCGTGA
TCACCCACAGCGGCAGGAAGGGCAGGTACGCCGTGGAGCCCTACCACGGC
AAGGTGGTGGTCCCCGAGGGCCACGCCATCCCCGTGCAGGACTTCCAGGC
CCTGAGCGAGAGCGCCACCATCGTGTATAACGAGAGGGAGTTCGTGAACA
GGTACCTGCACCACATCGCCACCCACGGCGGCGCCCTGAACACCGACGAG
GAGTACTACAAGACCGTGAAGCCCAGCGAGCACGACGGCGAGTACCTGTA
CGACATCGACAGGAAGCAGTGCGTGAAGAAGGAGCTGGTGACCGGCCTGG
GCCTGACCGGCGAGCTGGTGGACCCTCCCTTCCACGAGTTCGCCTACGAG
AGCCTGAGGACCAGGCCCGCCGCTCCCTACCAGGTGCCCACCATCGGCGT
GTACGGCGTGCCCGGCAGCGGCAAGAGCGGCATCATCAAGAGCGCCGTGA
CCAAGAAGGACCTGGTGGTGAGCGCCAAGAAGGAGAACTGCGCCGAGATC
ATCAGGGACGTGAAGAAGATGAAGGGCCTGGACGTGAACGCCAGGACCGT
GGACAGCGTGCTcCTGAACGGCTGCAAGCACCCCGTGGAGACACTGTATA
TCGACGAGGCCTTCGCCTGCCACGCCGGCACCCTGAGGGCCCTGATCGCC
ATCATCAGGCCCAAGAAGGCCGTGCTGTGCGGCGACCCCAAGCAGTGCGG
CTTCTTCAACATGATGTGCCTGAAGGTGCACTTCAACCACGAGATCTGCA
CCCAGGTGTTCCACAAGAGCATCAGCAGGCGGTGCACCAAGAGCGTGACC
AGCGTGGTGAGCACCCTGTTCTACGACAAGAAGATGAGGACCACCAACCC
CAAGGAGACAAAGATCGTGATCGACACCACCGGCAGCACCAAGCCCAAGC
AGGACGACCTGATCCTGACCTGCTTCAGGGGCTGGGTGAAGCAGCTGCAG
ATCGACTACAAGGGCAACGAGATCATGACCGCCGCCGCTAGCCAGGGCCT
GACCAGGAAGGGCGTGTACGCCGTGAGGTACAAGGTGAACGAGAATCCCC
TGTACGCCCCTACCAGCGAGCACGTGAACGTcCTGCTGACCAGGACCGAG
GACAGGATCGTGTGGAAGACCCTGGCCGGCGACCCCTGGATCAAGACCCT
GACCGCCAAGTACCCCGGCAACTTCACCGCCACCATCGAGGAGTGGCAGG
CCGAGCACGACGCCATCATGAGGCACATCCTGGAGAGGCCCGACCCCACC
GACGTGTTCCAGAACAAGGCCAACGTGTGCTGGGCCAAGGCCCTGGTGCC
CGTGCTGAAGACCGCCGGCATCGACATGACCACCGAGCAGTGGAACACCG
TGGACTACTTCGAGACAGACAAGGCCCACAGCGCCGAGATCGTGCTGAAC
CAGCTGTGCGTGAGGTTCTTCGGCCTGGACCTGGACAGCGGCCTGTTCAG
CGCCCCTACCGTGCCCCTGAGCATCAGGAACAACCACTGGGACAACAGCC
CCAGCCCCAACATGTACGGCCTGAACAAGGAGGTGGTGAGGCAGCTGAGC
AGGCGGTACCCTCAGCTGCCCAGGGCCGTGGCCACCGGCAGGGTGTACGA
CATGAACACCGGCACCCTGAGGAACTACGACCCCAGGATCAACCTGGTGC
CCGTGAACAGGCGGCTGCCaCACGCCCTGGTGCTGCACCACAACGAGCAC
CCTCAGAGCGACTTCAGCAGCTTCGTGAGCAAGCTGAAGGGCAGGACCGT
GCTGGTGGTGGGCGAGAAGCTGAGCGTGCCCGGCAAGATGGTGGACTGGC
TGAGCGACAGGCCCGAGGCCACCTTCCGGGCCAGGCTGGACCTGGGCATC
CCCGGCGACGTGCCCAAGTACGACATCATCTTCGTGAACGTGAGGACCCC
TTACAAGTACCACCACTACCAGCAGTGCGAGGACCACGCCATCAAGCTGA
GCATGCTGACCAAGAAGGCCTGCCTGCACCTGAACCCCGGCGGCACCTGC
GTGAGCATCGGCTACGGCTACGCCGACAGGGCCAGCGAGAGCATCATCGG
CGCCATCGCCAGGCTGTTCAAGTTCAGCAGGGTGTGCAAGCCCAAGAGCA
GCCTGGAGGAGACAGAGGTGCTGTTCGTGTTCATCGGCTACGACCGGAAG
GCCAGGACCCACAACCCCTACAAGCTGAGCAGCACCCTGACCAACATCTA
CACCGGCAGCAGGCTGCACGAGGCCGGCTGCGCCCCTAGCTACCACGTGG
TGAGGGGCGACATCGCCACCGCCACCGAGGGCGTGATCATCAACGCCGCC
AACAGCAAGGGCCAGCCCGGCGGCGGGGTGTGCGGCGCCCTGTATAAGAA
GTTCCCCGAGAGCTTCGACCTGCAGCCCATCGAGGTGGGCAAGGCCAGGC
TGGTGAAGGGCGCCGCCAAGCACATCATCCACGCCGTGGGCCCCAACTTC
AACAAGGTGAGCGAGGTGGAGGGCGACAAGCAGCTGGCCGAGGCCTACGA
GAGCATCGCCAAGATCGTGAACGACAACAACTACAAGAGCGTGGCCATCC
CTCTGCTGAGCACCGGCATCTTCAGCGGCAACAAGGACAGGCTGACCCAG
AGCCTGAACCACCTGCTGACCGCCCTGGACACCACCGACGCCGACGTGGC
CATCTACTGCAGGGACAAGAAGTGGGAGATGACCCTGAAGGAGGCCGTGG
CCAGGCGGGAGGCCGTGGAGGAGATCTGCATCAGCGACGACAGCAGCGTG
ACgGAGCCCGACGCCGAGCTGGTGAGGGTGCACCCCAAGAGCAGCCTGGC
CGGCAGGAAGGGCTACAGCACCAGCGACGGCAAGACCTTCAGCTACCTGG
AGGGCACCAAGTTCCACCAGGCCGCCAAGGACATCGCCGAGATCAACGCC
ATGTGGCCCGTGGCCACCGAGGCCAACGAGCAGGTGTGCATGTATATCCT
GGGCGAGAGCATGAGCAGCATCAGGAGCAAGTGCCCCGTGGAGGAGAGCG
AGGCCAGCACCCCTCCCAGCACCCTGCCCTGCCTGTGCATCCACGCCATG
ACCCCTGAGAGGGTGCAGCGGCTGAAGGCCAGCAGGCCCGAGCAGATCAC
CGTGTGCAGCAGCTTCCCTCTGCCCAAGTACcGGATCACCGGCGTGCAGA
AGATCCAGTGCAGCCAGCCCATCCTGTTCAGCCCCAAGGTGCCCGCCTAC
ATCCACCCCAGGAAGTACCTGGTGGAGACACCCCCCGTGGACGAGACACC
CGAGCCCAGCGCCGAGAACCAGAGCACCGAGGGCACCCCTGAGCAGCCTC
CCCTGATCACCGAGGACGAGACAAGGACCAGGACgCCcGAGCCCATCATC
ATTGAGGAGGAAGAGGAGGACAGCATCAGCCTGCTGAGCGACGGCCCCAC
CCACCAGGTGCTGCAGGTGGAGGCCGACATCCACGGCCCTCCCAGCGTGA
GCAGCTCCAGCTGGAGCATCCCTCACGCCAGCGACTTCGACGTGGACAGC
CTGAGCATCCTGGACACCCTGGAGGGCGCCAGCGTGACCAGCGGCGCCAC
CAGCGCCGAGACAAACAGCTACTTCGCCAAGAGCATGGAGTTCCTGGCCA
GGCCCGTGCCCGCCCCTAGGACCGTGTTCAGGAACCCTCCCCACCCCGCC
CCTAGGACCAGGACCCCTAGCCTGGCCCCTAGCAGGGCCTGCAGCAGGAC
CAGCCTGGTGAGCACCCCTCCCGGCGTGAACcGGGTGATCACCAGGGAGG
AGCTGGAGGCCCTGACCCCTAGCAGGACCCCTAGCAGGAGCGTGAGCAGG
ACCAGCCTGGTGAGCAACCCTCCCGGCGTGAACcGGGTGATCACCAGGGA
GGAGTTCGAGGCCTTCGTGGCCCAGCAGCAAAGGCGGTTCGACGCCGGCG
CCTACATCTTCAGCAGCGACACCGGCCAGGGCCACCTGCAGCAGAAGTCC
GTGAGGCAGACCGTGCTGAGCGAGGTGGTcCTGGAGAGGACgGAGCTGGA
GATCAGCTACGCCCCTAGGCTGGACCAGGAGAAGGAGGAGCTGCTGAGGA
AGAAGCTGCAGCTGAACCCCACCCCTGCCAACAGGAGCAGGTACCAGAGC
AGGAAGGTGGAGAACATGAAGGCCATCACCGCCAGGCGGATCCTGCAGGG
CCTGGGCCACTACCTGAAGGCCGAGGGCAAGGTGGAGTGCTACAGGACCC
TGCACCCCGTGCCCCTGTACTCCAGCTCCGTGAACAGGGCCTTCAGCAGC
CCCAAGGTGGCCGTGGAGGCCTGCAACGCCATGCTGAAGGAGAACTTCCC
CACCGTGGCCAGCTACTGCATCATCCCCGAGTACGACGCCTACCTGGACA
TGGTGGACGGCGCCAGCTGCTGCCTGGACACCGCCAGCTTCTGCCCCGCC
AAGCTGAGGAGCTTCCCCAAGAAGCACAGCTACCTGGAGCCCACCATCAG
GAGCGCCGTGCCCAGCGCCATCCAGAACACCCTGCAGAACGTGCTGGCCG
CCGCTACCAAGAGGAACTGCAACGTGACCCAGATGAGGGAGCTGCCCGTG
CTGGACAGCGCCGCCTTCAACGTGGAGTGCTTCAAGAAGTACGCCTGCAA
CAACGAGTACTGGGAGACATTCAAGGAGAACCCCATCAGGCTGACCGAGG
AGAACGTGGTGAACTACATCACCAAGCTGAAGGGCCCCAAGGCCGCCGCT
CTGTTCGCCAAGACCCACAACCTGAACATGCTcCAGGACATCCCTATGGA
CAGGTTCGTGATGGACCTGAAGAGGGACGTGAAGGTGACCCCTGGCACCA
AGCACACCGAGGAGAGGCCCAAGGTGCAGGTGATCCAGGCCGCCGACCCT
CTGGCCACCGCCTACCTGTGCGGCATCCACAGGGAGCTGGTGAGGCGGCT
GAACGCCGTcCTGCTGCCCAACATCCACACCCTGTTCGACATGAGCGCCG
AGGACTTCGACGCCATCATCGCCGAGCACTTCCAGCCCGGCGACTGCGTG
CTGGAGACAGACATCGCCAGCTTCGACAAGAGCGAGGACGACGCTATGGC
CCTGACCGCCCTGATGATCCTGGAGGACCTGGGCGTGGACGCCGAGCTGC
TGACCCTGATCGAGGCCGCCTTCGGCGAGATCAGCAGCATCCACCTGCCC
ACCAAGACCAAGTTCAAGTTCGGCGCCATGATGAAGTCCGGCATGTTCCT
GACCCTGTTCGTGAACACCGTGATCAACATCGTGATCGCCAGCAGGGTGC
TGCGGGAGAGGCTGACCGGCAGCCCCTGCGCCGCCTTCATCGGCGACGAC
AACATCGTGAAGGGCGTGAAGTCCGACAAGCTGATGGCCGACAGGTGCGC
CACCTGGCTGAACATGGAGGTGAAGATCATCGACGCCGTGGTGGGCGAGA
AGGCCCCTTACTTCTGCGGCGGCTTCATCCTGTGCGACAGCGTGACCGGC
ACCGCCTGCAGGGTGGCCGACCCTCTGAAGAGGCTGTTCAAGCTGGGCAA
GCCCCTGGCCGCCGACGACGAGCACGACGACGATAGGCGGAGGGCCCTGC
ACGAGGAGAGCACCAGGTGGAACcGGGTGGGCATCCTGAGCGAGCTGTGC
AAGGCCGTGGAGAGCAGGTACGAGACAGTGGGCACCAGCATCATCGTGAT
GGCCATGACCACCCTGGCCAGCAGCGTcAAGTCCTTCAGCTACCTGAGGG
GGGCCCCTATAACTCTCTACGGCTAACCTGAATGGACTACGACATAGTCT
AGTCCGCCAAGGCCGCCACCATGAAGGCTATCCTGGTGGTGCTGCTCTAC
ACCTTTGCCACAGCCAATGCTGACACCCTGTGTATTGGCTACCATGCCAA
CAACAGCACAGACACAGTGGACACAGTGTTGGAGAAGAATGTGACAGTGA
CCCACTCTGTGAACCTGTTGGAGGACAAACACAATGGCAAACTGTGTAAA
CTGAGGGGAGTGGCTCCACTGCACCTGGGCAAGTGTAACATTGCTGGCTG
GATTCTGGGCAACCCTGAGTGTGAGTCCCTGAGCACAGCCTCCTCCTGGT
CCTACATTGTGGAGACACCATCCTCTGACAATGGCACTTGTTACCCTGGA
GACTTCATTGACTATGAGGAACTGAGGGAACAACTTTCCTCTGTGTCCTC
CTTTGAGAGGTTTGAGATTTTTCCAAAGACCTCCTCCTGGCCAAACCATG
ACAGCAACAAGGGAGTGACAGCAGCCTGTCCACATGCTGGAGCCAAGTCC
TTCTACAAGAACCTGATTTGGCTGGTGAAGAAGGGCAACTCCTACCCAAA
ACTGAGCAAGTCCTACATCAATGACAAGGGCAAGGAGGTGCTGGTGCTGT
GGGGCATCCACCACCCAAGCACCTCTGCTGACCAACAGTCCCTCTACCAG
AATGCTGACGCCTATGTGTTTGTGGGCTCCAGCAGATACAGCAAGAAGTT
CAAGCCTGAGATTGCCATCAGACCAAAGGTGAGGGATcagGAGGGCAGGA
TGAACTACTACTGGACCCTGGTGGAACCTGGAGACAAGATTACCTTTGAG
GCTACAGGCAACCTGGTGGTGCCAAGATATGCCTTTGCTATGGAGAGGAA
TGCTGGCTCTGGCATCATCATCTCTGACACACCTGTCCATGACTGTAACA
CCACTTGTCAGACACCAAAGGGAGCCATCAACACCTCCCTGCCATTCCAG
AACATCCACCCAATCACCATTGGCAAGTGTCCAAAATATGTcAAGAGCAC
CAAACTGAGACTGGCTACAGGACTGAGGAACATCCCAAGCATCCAGAGCA
GGGGACTGTTTGGAGCCATTGCTGGCTTCATTGAGGGAGGCTGGACAGGG
ATGGTGGATGGCTGGTATGGCTACCACCACCAGAATGAACAGGGCTCTGG
CTATGCTGCTGACCTGAAAAGCACCCAGAATGCCATTGATGAGATTACCA
ACAAGGTGAACTCTGTGATTGAGAAGATGAACACCCAGTTCACAGCAGTG
GGCAAGGAGTTCAACCACTTGGAGAAGAGGATTGAGAACCTGAACAAGAA
GGTGGATGATGGCTTCCTGGACATCTGGACCTACAATGCTGAACTGCTGG
TGCTGTTGGAGAATGAGAGGACCCTGGACTACCATGACAGCAATGTGAAG
AACCTCTATGAGAAGGTGAGGAGCCAACTTAAAAACAATGCCAAGGAGAT
TGGCAATGGCTGTTTTGAGTTCTACCACAAGTGTGACAACACTTGTATGG
AGTCTGTGAAGAATGGCACCTATGACTACCCAAAATACTCTGAGGAGGCT
AAACTGAACAGGGAGGAGATTGATGGAGTGAAATTGGAGAGCACCAGGAT
TTACCAGATCCTGGCCATCTACAGCACCGTGGCCAGCAGCCTGGTGCTGG
TGGTGAGCCTGGGCGCCATCAGCTTCTGGATGTGCAGCAACGGCAGCTTG
CAGTGCAGGATCTGCATCTAAaCTCGAGTATGTTACGTGCAAAGGTGATT
GTCACCCCCCGAAAGACCATATTGTGACACACCCTCAGTATCACGCCCAA
ACATTTACAGCCGCGGTGTCAAAAACCGCGTGGACGTGGTTAACATCCCT
GCTGGGAGGATCAGCCGTAATTATTATAATTGGCTTGGTGCTGGCTACTA
TTGTGGCCATGTACGTGCTGACCAACCAGAAACATAATTGAATACAGCAG
CAATTGGCAAGCTGCTTACATAGAACTCGCGGCGATTGGCATGCCGCCTT
AAAATTTTTATTTTATTTTTTCTTTTCTTTTCCGAATCGGATTTTGTTTT
TAATATTTCAAAAAAAAAAAAAAAAAAAAAAAAATctagAAAAAAAAAAA
AAAAAAAAAAAAAAAAAAAAAAAAAAAAAAAAAAAAAAAAAAAAAAAAAA
AAAAAAAAAAAAAAAAAAAaaaaaaaaaaaaaaaaaaaa

SEQ ID NO:41 - 5′ UTR (of mARM3124/SEQ ID NO:40)

ATGGGCGGCGCATGAGAGAAGCCCAGACCAATTACCTACCCAAA

SEQ ID NO:42 - nsP1-nsP4 (of mARM3124/SEQ ID NO:40)

atggagaaagttcacgttgacatcgaggaagacagcccattcctcagagc
tttgcagcggagcttcccgcagtttgaggtagaagccaagcaggtcactg
ataatgaccatgctaatgccagagcgttttcgcatctggcttcaaaactg
atcgaaacggaggtggacccatccgacacgatccttgacattggaagtgc
gcccgcccgcagaatGTATTCTAAGCACAAGTATCATTGTATCtgtccga
tgagatgtgcggaagatccggacagattgtataagtatgcaactaagctg
aagaaaaactgtaaggaaataactgataaggaattggacaagaaaatgaa
ggagctggccgccgtcatgagcgaccctgacctggaaactgagactatgt
gcctccacgacgacgagtcgtgtcgctacgaagggcaagtcgctgtttac
caggatgtatacgcCGTcGACGGCCCCACCAGCCTGTACCACCAGGCCAA
CAAGGGCGTGAGGGTGGCCTACTGGATCGGCTTCGACACCACACCCTTCA
TGTTCAAGAACCTGGCCGGCGCCTACCCCAGCTACAGCACCAACTGGGCC
GACGAGACAGTGCTGACCGCCAGGAACATCGGCCTGTGCAGCAGCGACGT
GATGGAGAGGAGCCGGAGGGGCATGAGCATCCTGAGGAAGAAGTACCTGA
AGCCCAGCAACAACGTGCTGTTCAGCGTGGGCAGCACCATCTACCACGAG
AAGAGGGACCTGCTGAGGAGCTGGCACCTGCCCAGCGTGTTCCACCTGAG
GGGCAAGCAGAACTACACCTGCAGGTGCGAGACAATCGTGAGCTGCGACG
GCTACGTGGTGAAGAGGATCGCCATCAGCCCCGGCCTGTACGGCAAGCCC
AGCGGCTACGCCGCCACCATGCACAGGGAGGGCTTCCTGTGCTGCAAGGT
GACCGACACCCTGAACGGCGAGAGGGTGAGCTTCCCCGTGTGCACCTACG
TGCCCGCCACCCTGTGCGACCAGATGACCGGCATCCTGGCCACCGACGTG
AGCGCCGACGACGCCCAGAAGCTGCTGGTGGGCCTGAACCAGAGGATCGT
GGTGAACGGCAGGACCCAGAGGAACACCAACACCATGAAGAACTACCTGC
TGCCCGTGGTGGCCCAGGCCTTCGCCAGGTGGGCCAAGGAGTACAAGGAG
GACCAGGAGGACGAGAGGCCCCTGGGCCTGAGGGACcGaCAGCTGGTGAT
GGGCTGCTGCTGGGCCTTCAGGCGGCACAAGATCACCAGCATCTACAAGA
GGCCCGACACCCAGACCATCATCAAGGTGAACAGCGACTTCCACAGCTTC
GTGCTGCCCAGGATCGGCAGCAACACCCTGGAGATCGGCCTGAGGACCCG
GATCAGGAAGATGCTGGAGGAGCACAAGGAGCCCAGCCCTCTGATCACCG
CCGAGGACGTGCAGGAGGCCAAGTGCGCCGCCGACGAGGCCAAGGAGGTG
AGGGAGGCCGAGGAGCTGAGGGCCGCCCTGCCTCCCCTGGCCGCCGACGT
GGAGGAGCCCACCCTGGAGGCCGACGTGGACCTGATGCTGCAGGAGGCCG
GCGCCGGCAGCGTGGAGACACCCAGGGGCCTGATCAAGGTGACCAGCTAC
GACGGCGAGGACAAGATCGGCAGCTACGCCGTGCTcAGCCCTCAGGCCGT
GCTGAAGTCCGAGAAGCTGAGCTGCATCCACCCTCTGGCCGAGCAGGTGA
TCGTGATCACCCACAGCGGCAGGAAGGGCAGGTACGCCGTGGAGCCCTAC
CACGGCAAGGTGGTGGTCCCCGAGGGCCACGCCATCCCCGTGCAGGACTT
CCAGGCCCTGAGCGAGAGCGCCACCATCGTGTATAACGAGAGGGAGTTCG
TGAACAGGTACCTGCACCACATCGCCACCCACGGCGGCGCCCTGAACACC
GACGAGGAGTACTACAAGACCGTGAAGCCCAGCGAGCACGACGGCGAGTA
CCTGTACGACATCGACAGGAAGCAGTGCGTGAAGAAGGAGCTGGTGACCG
GCCTGGGCCTGACCGGCGAGCTGGTGGACCCTCCCTTCCACGAGTTCGCC
TACGAGAGCCTGAGGACCAGGCCCGCCGCTCCCTACCAGGTGCCCACCAT
CGGCGTGTACGGCGTGCCCGGCAGCGGCAAGAGCGGCATCATCAAGAGCG
CCGTGACCAAGAAGGACCTGGTGGTGAGCGCCAAGAAGGAGAACTGCGCC
GAGATCATCAGGGACGTGAAGAAGATGAAGGGCCTGGACGTGAACGCCAG
GACCGTGGACAGCGTGCTcCTGAACGGCTGCAAGCACCCCGTGGAGACAC
TGTATATCGACGAGGCCTTCGCCTGCCACGCCGGCACCCTGAGGGCCCTG
ATCGCCATCATCAGGCCCAAGAAGGCCGTGCTGTGCGGCGACCCCAAGCA
GTGCGGCTTCTTCAACATGATGTGCCTGAAGGTGCACTTCAACCACGAGA
TCTGCACCCAGGTGTTCCACAAGAGCATCAGCAGGCGGTGCACCAAGAGC
GTGACCAGCGTGGTGAGCACCCTGTTCTACGACAAGAAGATGAGGACCAC
CAACCCCAAGGAGACAAAGATCGTGATCGACACCACCGGCAGCACCAAGC
CCAAGCAGGACGACCTGATCCTGACCTGCTTCAGGGGCTGGGTGAAGCAG
CTGCAGATCGACTACAAGGGCAACGAGATCATGACCGCCGCCGCTAGCCA
GGGCCTGACCAGGAAGGGCGTGTACGCCGTGAGGTACAAGGTGAACGAGA
ATCCCCTGTACGCCCCTACCAGCGAGCACGTGAACGTcCTGCTGACCAGG
ACCGAGGACAGGATCGTGTGGAAGACCCTGGCCGGCGACCCCTGGATCAA
GACCCTGACCGCCAAGTACCCCGGCAACTTCACCGCCACCATCGAGGAGT
GGCAGGCCGAGCACGACGCCATCATGAGGCACATCCTGGAGAGGCCCGAC
CCCACCGACGTGTTCCAGAACAAGGCCAACGTGTGCTGGGCCAAGGCCCT
GGTGCCCGTGCTGAAGACCGCCGGCATCGACATGACCACCGAGCAGTGGA
ACACCGTGGACTACTTCGAGACAGACAAGGCCCACAGCGCCGAGATCGTG
CTGAACCAGCTGTGCGTGAGGTTCTTCGGCCTGGACCTGGACAGCGGCCT
GTTCAGCGCCCCTACCGTGCCCCTGAGCATCAGGAACAACCACTGGGACA
ACAGCCCCAGCCCCAACATGTACGGCCTGAACAAGGAGGTGGTGAGGCAG
CTGAGCAGGCGGTACCCTCAGCTGCCCAGGGCCGTGGCCACCGGCAGGGT
GTACGACATGAACACCGGCACCCTGAGGAACTACGACCCCAGGATCAACC
TGGTGCCCGTGAACAGGCGGCTGCCaCACGCCCTGGTGCTGCACCACAAC
GAGCACCCTCAGAGCGACTTCAGCAGCTTCGTGAGCAAGCTGAAGGGCAG
GACCGTGCTGGTGGTGGGCGAGAAGCTGAGCGTGCCCGGCAAGATGGTGG
ACTGGCTGAGCGACAGGCCCGAGGCCACCTTCCGGGCCAGGCTGGACCTG
GGCATCCCCGGCGACGTGCCCAAGTACGACATCATCTTCGTGAACGTGAG
GACCCCTTACAAGTACCACCACTACCAGCAGTGCGAGGACCACGCCATCA
AGCTGAGCATGCTGACCAAGAAGGCCTGCCTGCACCTGAACCCCGGCGGC
ACCTGCGTGAGCATCGGCTACGGCTACGCCGACAGGGCCAGCGAGAGCAT
CATCGGCGCCATCGCCAGGCTGTTCAAGTTCAGCAGGGTGTGCAAGCCCA
AGAGCAGCCTGGAGGAGACAGAGGTGCTGTTCGTGTTCATCGGCTACGAC
CGGAAGGCCAGGACCCACAACCCCTACAAGCTGAGCAGCACCCTGACCAA
CATCTACACCGGCAGCAGGCTGCACGAGGCCGGCTGCGCCCCTAGCTACC
ACGTGGTGAGGGGCGACATCGCCACCGCCACCGAGGGCGTGATCATCAAC
GCCGCCAACAGCAAGGGCCAGCCCGGCGGCGGGGTGTGCGGCGCCCTGTA
TAAGAAGTTCCCCGAGAGCTTCGACCTGCAGCCCATCGAGGTGGGCAAGG
CCAGGCTGGTGAAGGGCGCCGCCAAGCACATCATCCACGCCGTGGGCCCC
AACTTCAACAAGGTGAGCGAGGTGGAGGGCGACAAGCAGCTGGCCGAGGC
CTACGAGAGCATCGCCAAGATCGTGAACGACAACAACTACAAGAGCGTGG
CCATCCCTCTGCTGAGCACCGGCATCTTCAGCGGCAACAAGGACAGGCTG
ACCCAGAGCCTGAACCACCTGCTGACCGCCCTGGACACCACCGACGCCGA
CGTGGCCATCTACTGCAGGGACAAGAAGTGGGAGATGACCCTGAAGGAGG
CCGTGGCCAGGCGGGAGGCCGTGGAGGAGATCTGCATCAGCGACGACAGC
AGCGTGACgGAGCCCGACGCCGAGCTGGTGAGGGTGCACCCCAAGAGCAG
CCTGGCCGGCAGGAAGGGCTACAGCACCAGCGACGGCAAGACCTTCAGCT
ACCTGGAGGGCACCAAGTTCCACCAGGCCGCCAAGGACATCGCCGAGATC
AACGCCATGTGGCCCGTGGCCACCGAGGCCAACGAGCAGGTGTGCATGTA
TATCCTGGGCGAGAGCATGAGCAGCATCAGGAGCAAGTGCCCCGTGGAGG
AGAGCGAGGCCAGCACCCCTCCCAGCACCCTGCCCTGCCTGTGCATCCAC
GCCATGACCCCTGAGAGGGTGCAGCGGCTGAAGGCCAGCAGGCCCGAGCA
GATCACCGTGTGCAGCAGCTTCCCTCTGCCCAAGTACcGGATCACCGGCG
TGCAGAAGATCCAGTGCAGCCAGCCCATCCTGTTCAGCCCCAAGGTGCCC
GCCTACATCCACCCCAGGAAGTACCTGGTGGAGACACCCCCCGTGGACGA
GACACCCGAGCCCAGCGCCGAGAACCAGAGCACCGAGGGCACCCCTGAGC
AGCCTCCCCTGATCACCGAGGACGAGACAAGGACCAGGACgCCcGAGCCC
ATCATCATTGAGGAGGAAGAGGAGGACAGCATCAGCCTGCTGAGCGACGG
CCCCACCCACCAGGTGCTGCAGGTGGAGGCCGACATCCACGGCCCTCCCA
GCGTGAGCAGCTCCAGCTGGAGCATCCCTCACGCCAGCGACTTCGACGTG
GACAGCCTGAGCATCCTGGACACCCTGGAGGGCGCCAGCGTGACCAGCGG
CGCCACCAGCGCCGAGACAAACAGCTACTTCGCCAAGAGCATGGAGTTCC
TGGCCAGGCCCGTGCCCGCCCCTAGGACCGTGTTCAGGAACCCTCCCCAC
CCCGCCCCTAGGACCAGGACCCCTAGCCTGGCCCCTAGCAGGGCCTGCAG
CAGGACCAGCCTGGTGAGCACCCCTCCCGGCGTGAACcGGGTGATCACCA
GGGAGGAGCTGGAGGCCCTGACCCCTAGCAGGACCCCTAGCAGGAGCGTG
AGCAGGACCAGCCTGGTGAGCAACCCTCCCGGCGTGAACcGGGTGATCAC
CAGGGAGGAGTTCGAGGCCTTCGTGGCCCAGCAGCAAAGGCGGTTCGACG
CCGGCGCCTACATCTTCAGCAGCGACACCGGCCAGGGCCACCTGCAGCAG
AAGTCCGTGAGGCAGACCGTGCTGAGCGAGGTGGTcCTGGAGAGGACgGA
GCTGGAGATCAGCTACGCCCCTAGGCTGGACCAGGAGAAGGAGGAGCTGC
TGAGGAAGAAGCTGCAGCTGAACCCCACCCCTGCCAACAGGAGCAGGTAC
CAGAGCAGGAAGGTGGAGAACATGAAGGCCATCACCGCCAGGCGGATCCT
GCAGGGCCTGGGCCACTACCTGAAGGCCGAGGGCAAGGTGGAGTGCTACA
GGACCCTGCACCCCGTGCCCCTGTACTCCAGCTCCGTGAACAGGGCCTTC
AGCAGCCCCAAGGTGGCCGTGGAGGCCTGCAACGCCATGCTGAAGGAGAA
CTTCCCCACCGTGGCCAGCTACTGCATCATCCCCGAGTACGACGCCTACC
TGGACATGGTGGACGGCGCCAGCTGCTGCCTGGACACCGCCAGCTTCTGC
CCCGCCAAGCTGAGGAGCTTCCCCAAGAAGCACAGCTACCTGGAGCCCAC
CATCAGGAGCGCCGTGCCCAGCGCCATCCAGAACACCCTGCAGAACGTGC
TGGCCGCCGCTACCAAGAGGAACTGCAACGTGACCCAGATGAGGGAGCTG
CCCGTGCTGGACAGCGCCGCCTTCAACGTGGAGTGCTTCAAGAAGTACGC
CTGCAACAACGAGTACTGGGAGACATTCAAGGAGAACCCCATCAGGCTGA
CCGAGGAGAACGTGGTGAACTACATCACCAAGCTGAAGGGCCCCAAGGCC
GCCGCTCTGTTCGCCAAGACCCACAACCTGAACATGCTcCAGGACATCCC
TATGGACAGGTTCGTGATGGACCTGAAGAGGGACGTGAAGGTGACCCCTG
GCACCAAGCACACCGAGGAGAGGCCCAAGGTGCAGGTGATCCAGGCCGCC
GACCCTCTGGCCACCGCCTACCTGTGCGGCATCCACAGGGAGCTGGTGAG
GCGGCTGAACGCCGTcCTGCTGCCCAACATCCACACCCTGTTCGACATGA
GCGCCGAGGACTTCGACGCCATCATCGCCGAGCACTTCCAGCCCGGCGAC
TGCGTGCTGGAGACAGACATCGCCAGCTTCGACAAGAGCGAGGACGACGC
TATGGCCCTGACCGCCCTGATGATCCTGGAGGACCTGGGCGTGGACGCCG
AGCTGCTGACCCTGATCGAGGCCGCCTTCGGCGAGATCAGCAGCATCCAC
CTGCCCACCAAGACCAAGTTCAAGTTCGGCGCCATGATGAAGTCCGGCAT
GTTCCTGACCCTGTTCGTGAACACCGTGATCAACATCGTGATCGCCAGCA
GGGTGCTGCGGGAGAGGCTGACCGGCAGCCCCTGCGCCGCCTTCATCGGC
GACGACAACATCGTGAAGGGCGTGAAGTCCGACAAGCTGATGGCCGACAG
GTGCGCCACCTGGCTGAACATGGAGGTGAAGATCATCGACGCCGTGGTGG
GCGAGAAGGCCCCTTACTTCTGCGGCGGCTTCATCCTGTGCGACAGCGTG
ACCGGCACCGCCTGCAGGGTGGCCGACCCTCTGAAGAGGCTGTTCAAGCT
GGGCAAGCCCCTGGCCGCCGACGACGAGCACGACGACGATAGGCGGAGGG
CCCTGCACGAGGAGAGCACCAGGTGGAACcGGGTGGGCATCCTGAGCGAG
CTGTGCAAGGCCGTGGAGAGCAGGTACGAGACAGTGGGCACCAGCATCAT
CGTGATGGCCATGACCACCCTGGCCAGCAGCGTcAAGTCCTTCAGCTACC
TGAGGGGGGCCCCTATAACTCTCTACGGCTAA

SEQ ID NO:43 - Intergenic region (of mARM3124/SEQ ID NO:40)

CCTGAATGGACTACGACATAGTCTAGTCCGCCAAGGCCGCCACC

SEQ ID NO:44 - 3′ UTR (of mARM3124/SEQ ID NO:40), with poly-A

aCTCGAGTATGTTACGTGCAAAGGTGATTGTCACCCCCCGAAAGACCATA
TTGTGACACACCCTCAGTATCACGCCCAAACATTTACAGCCGCGGTGTCA
AAAACCGCGTGGACGTGGTTAACATCCCTGCTGGGAGGATCAGCCGTAAT
TATTATAATTGGCTTGGTGCTGGCTACTATTGTGGCCATGTACGTGCTGA
CCAACCAGAAACATAATTGAATACAGCAGCAATTGGCAAGCTGCTTACAT
AGAACTCGCGGCGATTGGCATGCCGCCTTAAAATTTTTATTTTATTTTTT
CTTTTCTTTTCCGAATCGGATTTTGTTTTTAATATTTCAAAAAAAAAAAA
AAAAAAAAAAAAATctagAAAAAAAAAAAAAAAAAAAAAAAAAAAAAAAA
AAAAAAAAAAAAAAAAAAAAAAAAAAAAAAAAAAAAAAAAAAAAAAAAaa
aaaaaaaaaaaaaaaaaa

SEQ ID NO:45 - 3′ UTR (of mARM3124/SEQ ID NO:40), without poly-A

aCTCGAGTATGTTACGTGCAAAGGTGATTGTCACCCCCCGAAAGACCATA
TTGTGACACACCCTCAGTATCACGCCCAAACATTTACAGCCGCGGTGTCA
AAAACCGCGTGGACGTGGTTAACATCCCTGCTGGGAGGATCAGCCGTAAT
TATTATAATTGGCTTGGTGCTGGCTACTATTGTGGCCATGTACGTGCTGA
CCAACCAGAAACATAATTGAATACAGCAGCAATTGGCAAGCTGCTTACAT
AGAACTCGCGGCGATTGGCATGCCGCCTTAAAATTTTTATTTTATTTTTT
CTTTTCTTTTCCGAATCGGATTTTGTTTTTAATATTTCAAAAAAAAAAAA
AAAAAAAAAAAAATctag

SEQ ID NO:46 - Transgene (nucleic acid sequence; mARM3124/SEQ ID NO:40)

ATGAAGGCTATCCTGGTGGTGCTGCTCTACACCTTTGCCACAGCCAATGC
TGACACCCTGTGTATTGGCTACCATGCCAACAACAGCACAGACACAGTGG
ACACAGTGTTGGAGAAGAATGTGACAGTGACCCACTCTGTGAACCTGTTG
GAGGACAAACACAATGGCAAACTGTGTAAACTGAGGGGAGTGGCTCCACT
GCACCTGGGCAAGTGTAACATTGCTGGCTGGATTCTGGGCAACCCTGAGT
GTGAGTCCCTGAGCACAGCCTCCTCCTGGTCCTACATTGTGGAGACACCA
TCCTCTGACAATGGCACTTGTTACCCTGGAGACTTCATTGACTATGAGGA
ACTGAGGGAACAACTTTCCTCTGTGTCCTCCTTTGAGAGGTTTGAGATTT
TTCCAAAGACCTCCTCCTGGCCAAACCATGACAGCAACAAGGGAGTGACA
GCAGCCTGTCCACATGCTGGAGCCAAGTCCTTCTACAAGAACCTGATTTG
GCTGGTGAAGAAGGGCAACTCCTACCCAAAACTGAGCAAGTCCTACATCA
ATGACAAGGGCAAGGAGGTGCTGGTGCTGTGGGGCATCCACCACCCAAGC
ACCTCTGCTGACCAACAGTCCCTCTACCAGAATGCTGACGCCTATGTGTT
TGTGGGCTCCAGCAGATACAGCAAGAAGTTCAAGCCTGAGATTGCCATCA
GACCAAAGGTGAGGGATcagGAGGGCAGGATGAACTACTACTGGACCCTG
GTGGAACCTGGAGACAAGATTACCTTTGAGGCTACAGGCAACCTGGTGGT
GCCAAGATATGCCTTTGCTATGGAGAGGAATGCTGGCTCTGGCATCATCA
TCTCTGACACACCTGTCCATGACTGTAACACCACTTGTCAGACACCAAAG
GGAGCCATCAACACCTCCCTGCCATTCCAGAACATCCACCCAATCACCAT
TGGCAAGTGTCCAAAATATGTcAAGAGCACCAAACTGAGACTGGCTACAG
GACTGAGGAACATCCCAAGCATCCAGAGCAGGGGACTGTTTGGAGCCATT
GCTGGCTTCATTGAGGGAGGCTGGACAGGGATGGTGGATGGCTGGTATGG
CTACCACCACCAGAATGAACAGGGCTCTGGCTATGCTGCTGACCTGAAAA
GCACCCAGAATGCCATTGATGAGATTACCAACAAGGTGAACTCTGTGATT
GAGAAGATGAACACCCAGTTCACAGCAGTGGGCAAGGAGTTCAACCACTT
GGAGAAGAGGATTGAGAACCTGAACAAGAAGGTGGATGATGGCTTCCTGG
ACATCTGGACCTACAATGCTGAACTGCTGGTGCTGTTGGAGAATGAGAGG
ACCCTGGACTACCATGACAGCAATGTGAAGAACCTCTATGAGAAGGTGAG
GAGCCAACTTAAAAACAATGCCAAGGAGATTGGCAATGGCTGTTTTGAGT
TCTACCACAAGTGTGACAACACTTGTATGGAGTCTGTGAAGAATGGCACC
TATGACTACCCAAAATACTCTGAGGAGGCTAAACTGAACAGGGAGGAGAT
TGATGGAGTGAAATTGGAGAGCACCAGGATTTACCAGATCCTGGCCATCT
ACAGCACCGTGGCCAGCAGCCTGGTGCTGGTGGTGAGCCTGGGCGCCATC
AGCTTCTGGATGTGCAGCAACGGCAGCTTGCAGTGCAGGATCTGCATCTA
A

SEQ ID NO:47 - Transgene (amino acid acid sequence; mARM3124/SEQ ID NO:40)

MKAILVVLLYTFATANADTLCIGYHANNSTDTVDTVLEKNVTVTHSVNLL
EDKHNGKLCKLRGVAPLHLGKCNIAGWILGNPECESLSTASSWSYIVETP
SSDNGTCYPGDFIDYEELREQLSSVSSFERFEIFPKTSSWPNHDSNKGVT
AACPHAGAKSFYKNLIWLVKKGNSYPKLSKSYINDKGKEVLVLWGIHHPS
TSADQQSLYQNADAYVFVGSSRYSKKFKPEIAIRPKVRDQEGRMNYYWTL
VEPGDKITFEATGNLVVPRYAFAMERNAGSGIIISDTPVHDCNTTCQTPK
GAINTSLPFQNIHPITIGKCPKYVKSTKLRLATGLRNIPSIQSRGLFGAI
AGFIEGGWTGMVDGWYGYHHQNEQGSGYAADLKSTQNAIDEITNKVNSVI
EKMNTQFTAVGKEFNHLEKRIENLNKKVDDGFLDIWTYNAELLVLLENER
TLDYHDSNVKNLYEKVRSQLKNNAKEIGNGCFEFYHKCDNTCMESVKNGT
YDYPKYSEEAKLNREEIDGVKLESTRIYQILAIYSTVASSLVLVVSLGAI
SFWMCSNGSLQCRICI

SEQ ID NO:48 - mARM3038 (mRNA HA (A/California/07/2009))

AGGAAACUUAAGUCAACACAACAUAUACAAAACAAACGAAUCUCAAGCAA
UCAAGCAUUCUACUUCUAUUGCAGCAAUUUAAAUCAUUUCUUUUAAAGCA
AAAGCAAUUUUCUGAAAAUUUUCACCAUUUACGAACGAUAGCCACCAUGA
AGGCUAUCCUGGUGGUGCUGCUCUACACCUUUGCCACAGCCAAUGCUGAC
ACCCUGUGUAUUGGCUACCAUGCCAACAACAGCACAGACACAGUGGACAC
AGUGUUGGAGAAGAAUGUGACAGUGACCCACUCUGUGAACCUGUUGGAGG
ACAAACACAAUGGCAAACUGUGUAAACUGAGGGGAGUGGCUCCACUGCAC
CUGGGCAAGUGUAACAUUGCUGGCUGGAUUCUGGGCAACCCUGAGUGUGA
GUCCCUGAGCACAGCCUCCUCCUGGUCCUACAUUGUGGAGACACCAUCCU
CUGACAAUGGCACUUGUUACCCUGGAGACUUCAUUGACUAUGAGGAACUG
AGGGAACAACUUUCCUCUGUGUCCUCCUUUGAGAGGUUUGAGAUUUUUCC
AAAGACCUCCUCCUGGCCAAACCAUGACAGCAACAAGGGAGUGACAGCAG
CCUGUCCACAUGCUGGAGCCAAGUCCUUCUACAAGAACCUGAUUUGGCUG
GUGAAGAAGGGCAACUCCUACCCAAAACUGAGCAAGUCCUACAUCAAUGA
CAAGGGCAAGGAGGUGCUGGUGCUGUGGGGCAUCCACCACCCAAGCACCU
CUGCUGACCAACAGUCCCUCUACCAGAAUGCUGACGCCUAUGUGUUUGUG
GGCUCCAGCAGAUACAGCAAGAAGUUCAAGCCUGAGAUUGCCAUCAGACC
AAAGGUGAGGGAUCAGGAGGGCAGGAUGAACUACUACUGGACCCUGGUGG
AACCUGGAGACAAGAUUACCUUUGAGGCUACAGGCAACCUGGUGGUGCCA
AGAUAUGCCUUUGCUAUGGAGAGGAAUGCUGGCUCUGGCAUCAUCAUCUC
UGACACACCUGUCCAUGACUGUAACACCACUUGUCAGACACCAAAGGGAG
CCAUCAACACCUCCCUGCCAUUCCAGAACAUCCACCCAAUCACCAUUGGC
AAGUGUCCAAAAUAUGUCAAGAGCACCAAACUGAGACUGGCUACAGGACU
GAGGAACAUCCCAAGCAUCCAGAGCAGGGGACUGUUUGGAGCCAUUGCUG
GCUUCAUUGAGGGAGGCUGGACAGGGAUGGUGGAUGGCUGGUAUGGCUAC
CACCACCAGAAUGAACAGGGCUCUGGCUAUGCUGCUGACCUGAAAAGCAC
CCAGAAUGCCAUUGAUGAGAUUACCAACAAGGUGAACUCUGUGAUUGAGA
AGAUGAACACCCAGUUCACAGCAGUGGGCAAGGAGUUCAACCACUUGGAG
AAGAGGAUUGAGAACCUGAACAAGAAGGUGGAUGAUGGCUUCCUGGACAU
CUGGACCUACAAUGCUGAACUGCUGGUGCUGUUGGAGAAUGAGAGGACCC
UGGACUACCAUGACAGCAAUGUGAAGAACCUCUAUGAGAAGGUGAGGAGC
CAACUUAAAAACAAUGCCAAGGAGAUUGGCAAUGGCUGUUUUGAGUUCUA
CCACAAGUGUGACAACACUUGUAUGGAGUCUGUGAAGAAUGGCACCUAUG
ACUACCCAAAAUACUCUGAGGAGGCUAAACUGAACAGGGAGGAGAUUGAU
GGAGUGAAAUUGGAGAGCACCAGGAUUUACCAGAUCCUGGCCAUCUACAG
CACCGUGGCCAGCAGCCUGGUGCUGGUGGUGAGCCUGGGCGCCAUCAGCU
UCUGGAUGUGCAGCAACGGCAGCUUGCAGUGCAGGAUCUGCAUCUAAACU
CGAGCUAGUGACUGACUAGGAUCUGGUUACCACUAAACCAGCCUCAAGAA
CACCCGAAUGGAGUCUCUAAGCUACAUAAUACCAACUUACACUUACAAAA
UGUUGUCCCCCAAAAUGUAGCCAUUCGUAUCUGCUCCUAAUAAAAAGAAA
GUUUCUUCACAUUCUAGAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAA

SEQ ID NO:49 - 5′ UTR (of mARM3038/SEQ ID NO:48)

AGGAAACTTAAGTCAACACAACATATACAAAACAAACGAATCTCAAGCAA
TCAAGCATTCTACTTCTATTGCAGCAATTTAAATCATTTCTTTTAAAGCA
AAAGCAATTTTCTGAAAATTTTCACCATTTACGAACGATAGCCACC

SEQ ID NO:50 - 3′ UTR (of mARM3038/SEQ ID NO:48), with poly A

ACTCGAGCTAGTGACTGACTAGGATCTGGTTACCACTAAACCAGCCTCAA
GAACACCCGAATGGAGTCTCTAAGCTACATAATACCAACTTACACTTACA
AAATGTTGTCCCCCAAAATGTAGCCATTCGTATCTGCTCCTAATAAAAAG
AAAGTTTCTTCACATTCTAGAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAA

SEQ ID NO:51 - 3′ UTR (of mARM3038/SEQ ID NO:48), without poly A

ACTCGAGCTAGTGACTGACTAGGATCTGGTTACCACTAAACCAGCCTCAA
GAACACCCGAATGGAGTCTCTAAGCTACATAATACCAACTTACACTTACA
AAATGTTGTCCCCCAAAATGTAGCCATTCGTATCTGCTCCTAATAAAAAG
AAAGTTTCTTCACATTCTAG

SEQ ID NO:52 - Transgene (nucleic acid sequence; mARM3038/SEQ ID NO:48)

AUGAAGGCUAUCCUGGUGGUGCUGCUCUACACCUUUGCCACAGCCAAUGC
UGACACCCUGUGUAUUGGCUACCAUGCCAACAACAGCACAGACACAGUGG
ACACAGUGUUGGAGAAGAAUGUGACAGUGACCCACUCUGUGAACCUGUUG
GAGGACAAACACAAUGGCAAACUGUGUAAACUGAGGGGAGUGGCUCCACU
GCACCUGGGCAAGUGUAACAUUGCUGGCUGGAUUCUGGGCAACCCUGAGU
GUGAGUCCCUGAGCACAGCCUCCUCCUGGUCCUACAUUGUGGAGACACCA
UCCUCUGACAAUGGCACUUGUUACCCUGGAGACUUCAUUGACUAUGAGGA
ACUGAGGGAACAACUUUCCUCUGUGUCCUCCUUUGAGAGGUUUGAGAUUU
UUCCAAAGACCUCCUCCUGGCCAAACCAUGACAGCAACAAGGGAGUGACA
GCAGCCUGUCCACAUGCUGGAGCCAAGUCCUUCUACAAGAACCUGAUUUG
GCUGGUGAAGAAGGGCAACUCCUACCCAAAACUGAGCAAGUCCUACAUCA
AUGACAAGGGCAAGGAGGUGCUGGUGCUGUGGGGCAUCCACCACCCAAGC
ACCUCUGCUGACCAACAGUCCCUCUACCAGAAUGCUGACGCCUAUGUGUU
UGUGGGCUCCAGCAGAUACAGCAAGAAGUUCAAGCCUGAGAUUGCCAUCA
GACCAAAGGUGAGGGAUcagGAGGGCAGGAUGAACUACUACUGGACCCUG
GUGGAACCUGGAGACAAGAUUACCUUUGAGGCUACAGGCAACCUGGUGGU
GCCAAGAUAUGCCUUUGCUAUGGAGAGGAAUGCUGGCUCUGGCAUCAUCA
UCUCUGACACACCUGUCCAUGACUGUAACACCACUUGUCAGACACCAAAG
GGAGCCAUCAACACCUCCCUGCCAUUCCAGAACAUCCACCCAAUCACCAU
UGGCAAGUGUCCAAAAUAUGUcAAGAGCACCAAACUGAGACUGGCUACAG
GACUGAGGAACAUCCCAAGCAUCCAGAGCAGGGGACUGUUUGGAGCCAUU
GCUGGCUUCAUUGAGGGAGGCUGGACAGGGAUGGUGGAUGGCUGGUAUGG
CUACCACCACCAGAAUGAACAGGGCUCUGGCUAUGCUGCUGACCUGAAAA
GCACCCAGAAUGCCAUUGAUGAGAUUACCAACAAGGUGAACUCUGUGAUU
GAGAAGAUGAACACCCAGUUCACAGCAGUGGGCAAGGAGUUCAACCACUU
GGAGAAGAGGAUUGAGAACCUGAACAAGAAGGUGGAUGAUGGCUUCCUGG
ACAUCUGGACCUACAAUGCUGAACUGCUGGUGCUGUUGGAGAAUGAGAGG
ACCCUGGACUACCAUGACAGCAAUGUGAAGAACCUCUAUGAGAAGGUGAG
GAGCCAACUUAAAAACAAUGCCAAGGAGAUUGGCAAUGGCUGUUUUGAGU
UCUACCACAAGUGUGACAACACUUGUAUGGAGUCUGUGAAGAAUGGCACC
UAUGACUACCCAAAAUACUCUGAGGAGGCUAAACUGAACAGGGAGGAGAU
UGAUGGAGUGAAAUUGGAGAGCACCAGGAUUUACCAGAUCCUGGCCAUCU
ACAGCACCGUGGCCAGCAGCCUGGUGCUGGUGGUGAGCCUGGGCGCCAUC
AGCUUCUGGAUGUGCAGCAACGGCAGCUUGCAGUGCAGGAUCUGCAUCUA
A

SEQ ID NO:53 - Transgene (amino acid acid sequence; mARM3038/SEQ ID NO:48)

MKAILVVLLYTFATANADTLCIGYHANNSTDTVDTVLEKNVTVTHSVNLL
EDKHNGKLCKLRGVAPLHLGKCNIAGWILGNPECESLSTASSWSYIVETP
SSDNGTCYPGDFIDYEELREQLSSVSSFERFEIFPKTSSWPNHDSNKGVT
AACPHAGAKSFYKNLIWLVKKGNSYPKLSKSYINDKGKEVLVLWGIHHPS
TSADQQSLYQNADAYVFVGSSRYSKKFKPEIAIRPKVRDQEGRMNYYWTL
VEPGDKITFEATGNLVVPRYAFAMERNAGSGIIISDTPVHDCNTTCQTPK
GAINTSLPFQNIHPITIGKCPKYVKSTKLRLATGLRNIPSIQSRGLFGAI
AGFIEGGWTGMVDGWYGYHHQNEQGSGYAADLKSTQNAIDEITNKVNSVI
EKMNTQFTAVGKEFNHLEKRIENLNKKVDDGFLDIWTYNAELLVLLENER
TLDYHDSNVKNLYEKVRSQLKNNAKEIGNGCFEFYHKCDNTCMESVKNGT
YDYPKYSEEAKLNREEIDGVKLESTRIYQILAIYSTVASSLVLVVSLGAI
SFWMCSNGSLQCRICI*

SEQ ID NO:54 - mmu-miR-451a

AAACCGUUACCAUUACUGAGUU

SEQ ID NO:55 - mmu-miR-191-5p

CAACGGAAUCCCAAAAGCAGCUG

SEQ ID NO:56 - mmu-miR-181a-5p

AACAUUCAACGCUGUCGGUGAGU

SEQ ID NO:57 - mmu-miR-99b-5p

CACCCGUAGAACCGACCUUGCG

SEQ ID NO:58 - mmu-miR-10a-5p

UACCCUGUAGAUCCGAAUUUGUG

SEQ ID NO:59 - mmu-miR-10b-5p

UACCCUGUAGAACCGAAUUUGUG

SEQ ID NO:60 - mmu-miR-193b-3p

AACUGGCCCACAAAGUCCCGCU

SEQ ID NO:61 - mmu-miR-22-3p

AAGCUGCCAGUUGAAGAACUGU

SEQ ID NO:62 - mmu-miR-126a-5p

CAUUAUUACUUUUGGUACGCG

SEQ ID NO:63 - mmu-miR-92a-3p

UAUUGCACUUGUCCCGGCCUG

SEQ ID NO:64 - mmu-miR-125a-5p

UCCCUGAGACCCUUUAACCUGUGA

SEQ ID NO:65 - mmu-miR-378a-3p

ACUGGACUUGGAGUCAGAAGG

SEQ ID NO:66 - mmu-miR-143-3p

UGAGAUGAAGCACUGUAGCUC

SEQ ID NO:67 - mmu-let-7a-5p

UGAGGUAGUAGGUUGUAUAGUU

SEQ ID NO:68 - mmu-let-7b-5p

UGAGGUAGUAGGUUGUGUGGUU

SEQ ID NO:69 - mmu-let-7c-5p

UGAGGUAGUAGGUUGUAUGGUU

SEQ ID NO:70 - mmu-let-7f-5p

UGAGGUAGUAGAUUGUAUAGUU

SEQ ID NO:71 - mmu-miR-126b-3p

CGCGUACCAAAAGUAAUAAUGUG

SEQ ID NO:72 - mmu-miR-423-3p

AGCUCGGUCUGAGGCCCCUCAGU

SEQ ID NO:73 - mmu-miR-30a-5p

UGUAAACAUCCUCGACUGGAAG

SEQ ID NO:74 - mmu-miR-30d-5p

UGUAAACAUCCCCGACUGGAAG

SEQ ID NO:75 - mmu-miR-30e-5p

UGUAAACAUCCUUGACUGGAAG

SEQ ID NO:76 - mmu-miR-26a-5p

UUCAAGUAAUCCAGGAUAGGCU

SEQ ID NO:77 - mmu-miR-27b-3p

UUCACAGUGGCUAAGUUCUGC

SEQ ID NO:78 - mmu-miR-133a-3p.1

UUGGUCCCCUUCAACCAGCUG

SEQ ID NO:79 - mmu-miR-133a-3p.2

UUUGGUCCCCUUCAACCAGCUG

SEQ ID NO:80 - hsa-miR-486-5p

UCCUGUACUGAGCUGCCCCGAG

SEQ ID NO:81 - hsa-miR-486-3p

CGGGGCAGCUCAGUACAGGAU

SEQ ID NO:82 - hsa-miR-451a

AAACCGUUACCAUUACUGAGUU

SEQ ID NO:83 - hsa-miR-423-3p

AGCUCGGUCUGAGGCCCCUCAGU

SEQ ID NO:84 - hsa-miR-378a-3p

ACUGGACUUGGAGUCAGAAGGC

SEQ ID NO:85 - hsa-miR-193b-3p

AACUGGCCCUCAAAGUCCCGCU

SEQ ID NO:86 - hsa-miR-191-5p

CAACGGAAUCCCAAAAGCAGCUG

SEQ ID NO:87 - hsa-miR-181a-5p

AACAUUCAACGCUGUCGGUGAGU

SEQ ID NO:88 - hsa-miR-143-3p

UGAGAUGAAGCACUGUAGCUC

SEQ ID NO:89 - hsa-miR-133a-3p.2

UUUGGUCCCCUUCAACCAGCUG

SEQ ID NO:90 - hsa-miR-133a-3p.1

UUGGUCCCCUUCAACCAGCUG

SEQ ID NO:91 - hsa-miR-125a-5p

UCCCUGAGACCCUUUAACCUGUGA

SEQ ID NO:92 - hsa-miR-101-3p.2

GUACAGUACUGUGAUAACUGA

SEQ ID NO:93 - hsa-miR-101-3p.1

UACAGUACUGUGAUAACUGAA

SEQ ID NO:94 - hsa-miR-99b-5p

CACCCGUAGAACCGACCUUGCG

SEQ ID NO:95 - hsa-miR-30a-5p

UGUAAACAUCCUCGACUGGAAG

SEQ ID NO:96 - hsa-miR-30d-5p

UGUAAACAUCCCCGACUGGAAG

SEQ ID NO:97 - hsa-miR-30e-5p

UGUAAACAUCCUUGACUGGAAG

SEQ ID NO:98 - hsa-miR-27b-3p

UUCACAGUGGCUAAGUUCUGC

SEQ ID NO:99 - hsa-miR-26a-5p

UUCAAGUAAUCCAGGAUAGGCU

SEQ ID NO:100 - hsa-miR-92a-3p

UAUUGCACUUGUCCCGGCCUGU

SEQ ID NO:101 - hsa-miR-22-3p

AAGCUGCCAGUUGAAGAACUGU

SEQ ID NO:102- hsa-miR-10a-5p

UACCCUGUAGAUCCGAAUUUGUG

SEQ ID NO:103- hsa-miR-10b-5p

UACCCUGUAGAACCGAAUUUGUG

SEQ ID NO:104 - hsa-let-7a-5p

UGAGGUAGUAGGUUGUAUAGUU

SEQ ID NO:105 - hsa-let-7b-5p

UGAGGUAGUAGGUUGUGUGGUU

SEQ ID NO:106 - hsa-let-7c-5p

UGAGGUAGUAGGUUGUAUGGUU

SEQ ID NO:107 - hsa-let-7f-5p

UGAGGUAGUAGAUUGUAUAGUU

SEQ ID NO:108 - mmu-miR-191-5p

CAACGGAAUCCCAAAAGCAGCUG

SEQ ID NO:109 - mmu-miR-181a-5p

AACAUUCAACGCUGUCGGUGAGU

SEQ ID NO:110 - mmu-miR-181b-5p

AACAUUCAUUGCUGUCGGUGGGU

SEQ ID NO:111 - mmu-miR-99b-5p

CACCCGUAGAACCGACCUUGCG

SEQ ID NO:112 - mmu-miR-10a-5p

UACCCUGUAGAUCCGAAUUUGUG

SEQ ID NO:113 - mmu-miR-29a-3p

UAGCACCAUCUGAAAUCGGUUA

SEQ ID NO:114- mmu-miR-16-5p

UAGCAGCACGUAAAUAUUGGCG

SEQ ID NO:115 - mmu-miR-22-3p

AAGCUGCCAGUUGAAGAACUGU

SEQ ID NO:116 - mmu-miR-21a-5p

UAGCUUAUCAGACUGAUGUUGA

SEQ ID NO:117 - mmu-miR-142a-5p

CAUAAAGUAGAAAGCACUACU

SEQ ID NO:118 - mmu-miR-25-3p

CAUUGCACUUGUCUCGGUCUGA

SEQ ID NO:119 - mmu-miR-92a-3p

UAUUGCACUUGUCCCGGCCUG

SEQ ID NO:120 - mmu-miR-148a-3p

UCAGUGCACUACAGAACUUUGU

SEQ ID NO:121 - mmu-miR-378a-3p

ACUGGACUUGGAGUCAGAAGG

SEQ ID NO:122 - mmu-miR-146b-5p

UGAGAACUGAAUUCCAUAGGCU

SEQ ID NO:123 - mmu-miR-27b-5p

AGAGCUUAGCUGAUUGGUGAAC

SEQ ID NO:124 - mmu-let-7a-5p

UGAGGUAGUAGGUUGUAUAGUU

SEQ ID NO:125 - mmu-let-7f-5p

UGAGGUAGUAGAUUGUAUAGUU

SEQ ID NO:126 - mmu-let-7g-5p

UGAGGUAGUAGUUUGUACAGUU

SEQ ID NO:127 - mmu-let-7i-5p

UGAGGUAGUAGUUUGUGCUGUU

SEQ ID NO:128 - mmu-miR-103-3p

AGCAGCAUUGUACAGGGCUAUGA

SEQ ID NO:129 - mmu-miR-221-3p

AGCUACAUUGUCUGCUGGGUUUC

SEQ ID NO:130 - mmu-miR-222-3p

AGCUACAUCUGGCUACUGGGU

SEQ ID NO:131- mmu-miR-24-3p

UGGCUCAGUUCAGCAGGAACAG

SEQ ID NO:132 - mmu-miR-27a-5p

AGGGCUUAGCUGCUUGUGAGCA

SEQ ID NO:133 - mmu-miR-30d-5p

UGUAAACAUCCCCGACUGGAAG

SEQ ID NO:134 - mmu-miR-223-3p

UGUCAGUUUGUCAAAUACCCCA

SEQ ID NO:135 - mmu-miR-223-5p

CGUGUAUUUGACAAGCUGAGUUG

SEQ ID NO:136 - mmu-miR-155-5p

UUAAUGCUAAUUGUGAUAGGGGU

SEQ ID NO:137 - mmu-miR-26a-5p

UUCAAGUAAUCCAGGAUAGGCU

SEQ ID NO:138 - mmu-miR-26b-5p

UUCAAGUAAUUCAGGAUAGGU

SEQ ID NO:139 - mmu-miR-27a-3p

UUCACAGUGGCUAAGUUCCGC

SEQ ID NO:140 - mmu-miR-27b-3p

UUCACAGUGGCUAAGUUCUGC

SEQ ID NO:141 - hsa-miR-423-5p

UGAGGGGCAGAGAGCGAGACUUU

SEQ ID NO:142 - hsa-miR-423-3p

AGCUCGGUCUGAGGCCCCUCAGU

SEQ ID NO:143 - hsa-miR-378a-3p

ACUGGACUUGGAGUCAGAAGGC

SEQ ID NO:144 - hsa-miR-342-3p

UCUCACACAGAAAUCGCACCCGU

SEQ ID NO:145 - hsa-miR-223-5p

CGUGUAUUUGACAAGCUGAGUU

SEQ ID NO:146 - hsa-miR-223-3p

UGUCAGUUUGUCAAAUACCCCA

SEQ ID NO:147 - hsa-miR-191-5p

CAACGGAAUCCCAAAAGCAGCUG

SEQ ID NO:148 - hsa-miR-186-5p

CAAAGAAUUCUCCUUUUGGGCU

SEQ ID NO:149 - hsa-miR-181a-5p

AACAUUCAACGCUGUCGGUGAGU

SEQ ID NO:150 - hsa-miR-146b-5p

UGAGAACUGAAUUCCAUAGGCU

SEQ ID NO:151 - hsa-miR-142-5p

CAUAAAGUAGAAAGCACUACU

SEQ ID NO:152 - hsa-miR-142-3p.2

GUAGUGUUUCCUACUUUAUGGA

SEQ ID NO:153 - hsa-miR-142-3p.1

UGUAGUGUUUCCUACUUUAUGGA

SEQ ID NO:154 - hsa-miR-140-3p.2

UACCACAGGGUAGAACCACGG

SEQ ID NO:155 - hsa-miR-140-3p.1

ACCACAGGGUAGAACCACGGAC

SEQ ID NO:156 - hsa-miR-103a-3p

AGCAGCAUUGUACAGGGCUAUGA

SEQ ID NO:157 - hsa-miR-107

AGCAGCAUUGUACAGGGCUAUCA

SEQ ID NO:158 - hsa-miR-30a-5p

UGUAAACAUCCUCGACUGGAAG

SEQ ID NO:159 - hsa-miR-30c-5p

UGUAAACAUCCUACACUCUCAGC

SEQ ID NO:160 - hsa-miR-30d-5p

UGUAAACAUCCCCGACUGGAAG

SEQ ID NO:161 - hsa-miR-30e-5p

UGUAAACAUCCUUGACUGGAAG

SEQ ID NO:162 - hsa-miR-28-3p

CACUAGAUUGUGAGCUCCUGGA

SEQ ID NO:163 - hsa-miR-27b-5p

AGAGCUUAGCUGAUUGGUGAAC

SEQ ID NO:164 - hsa-miR-27a-5p

AGGGCUUAGCUGCUUGUGAGCA

SEQ ID NO:165 - hsa-miR-27a-3p

UUCACAGUGGCUAAGUUCCGC

SEQ ID NO:166 - hsa-miR-27b-3p

UUCACAGUGGCUAAGUUCUGC

SEQ ID NO:167 - hsa-miR-26a-5p

UUCAAGUAAUCCAGGAUAGGCU

SEQ ID NO:168 - hsa-miR-26b-5p

UUCAAGUAAUUCAGGAUAGGU

SEQ ID NO:169 - hsa-miR-25-3p

CAUUGCACUUGUCUCGGUCUGA

SEQ ID NO:170 - hsa-miR-92a-3p

UAUUGCACUUGUCCCGGCCUGU

SEQ ID NO:171- hsa-miR-24-3p

UGGCUCAGUUCAGCAGGAACAG

SEQ ID NO:172 - hsa-miR-22-3p

AAGCUGCCAGUUGAAGAACUGU

SEQ ID NO:173 - hsa-miR-21-5p

UAGCUUAUCAGACUGAUGUUGA

SEQ ID NO:174 - hsa-miR-21-3p

CAACACCAGUCGAUGGGCUGU

SEQ ID NO:175- hsa-miR-16-5p

UAGCAGCACGUAAAUAUUGGCG

SEQ ID NO:176 - hsa-let-7a-5p

UGAGGUAGUAGGUUGUAUAGUU

SEQ ID NO:177 - hsa-let-7b-5p

UGAGGUAGUAGGUUGUGUGGUU

SEQ ID NO:178 - hsa-let-7c-5p

UGAGGUAGUAGGUUGUAUGGUU

SEQ ID NO:179 - hsa-let-7d-5p

AGAGGUAGUAGGUUGCAUAGUU

SEQ ID NO:180 - hsa-let-7e-5p

UGAGGUAGGAGGUUGUAUAGUU

SEQ ID NO:181 - hsa-let-7f-5p

UGAGGUAGUAGAUUGUAUAGUU

SEQ ID NO:182 - hsa-let-7g-5p

UGAGGUAGUAGUUUGUACAGUU

SEQ ID NO:183 - hsa-let-7i-5p

UGAGGUAGUAGUUUGUGCUGUU

SEQ ID NO:184 - hsa-miR-98-5p

UGAGGUAGUAAGUUGUAUUGUU

SEQ ID NO:185 - Codon optimized region of nsP1-4 (nucleotide 463 to nucleotide 7455)

GCCGTGGACGGCCCCACCAGCCTGTACCACCAGGCCAACAAGGGCGTGAG
GGTGGCCTACTGGATCGGCTTCGACACCACACCCTTCATGTTCAAGAACC
TGGCCGGCGCCTACCCCAGCTACAGCACCAACTGGGCCGACGAGACAGTG
CTGACCGCCAGGAACATCGGCCTGTGCAGCAGCGACGTGATGGAGAGGAG
CCGGAGGGGCATGAGCATCCTGAGGAAGAAGTACCTGAAGCCCAGCAACA
ACGTGCTGTTCAGCGTGGGCAGCACCATCTACCACGAGAAGAGGGACCTG
CTGAGGAGCTGGCACCTGCCCAGCGTGTTCCACCTGAGGGGCAAGCAGAA
CTACACCTGCAGGTGCGAGACAATCGTGAGCTGCGACGGCTACGTGGTGA
AGAGGATCGCCATCAGCCCCGGCCTGTACGGCAAGCCCAGCGGCTACGCC
GCCACCATGCACAGGGAGGGCTTCCTGTGCTGCAAGGTGACCGACACCCT
GAACGGCGAGAGGGTGAGCTTCCCCGTGTGCACCTACGTGCCCGCCACCC
TGTGCGACCAGATGACCGGCATCCTGGCCACCGACGTGAGCGCCGACGAC
GCCCAGAAGCTGCTGGTGGGCCTGAACCAGAGGATCGTGGTGAACGGCAG
GACCCAGAGGAACACCAACACCATGAAGAACTACCTGCTGCCCGTGGTGG
CCCAGGCCTTCGCCAGGTGGGCCAAGGAGTACAAGGAGGACCAGGAGGAC
GAGAGGCCCCTGGGCCTGAGGGACAGGCAGCTGGTGATGGGCTGCTGCTG
GGCCTTCAGGCGGCACAAGATCACCAGCATCTACAAGAGGCCCGACACCC
AGACCATCATCAAGGTGAACAGCGACTTCCACAGCTTCGTGCTGCCCAGG
ATCGGCAGCAACACCCTGGAGATCGGCCTGAGGACCCGGATCAGGAAGAT
GCTGGAGGAGCACAAGGAGCCCAGCCCTCTGATCACCGCCGAGGACGTGC
AGGAGGCCAAGTGCGCCGCCGACGAGGCCAAGGAGGTGAGGGAGGCCGAG
GAGCTGAGGGCCGCCCTGCCTCCCCTGGCCGCCGACGTGGAGGAGCCCAC
CCTGGAGGCCGACGTGGACCTGATGCTGCAGGAGGCCGGCGCCGGCAGCG
TGGAGACACCCAGGGGCCTGATCAAGGTGACCAGCTACGACGGCGAGGAC
AAGATCGGCAGCTACGCCGTGCTGAGCCCTCAGGCCGTGCTGAAGTCCGA
GAAGCTGAGCTGCATCCACCCTCTGGCCGAGCAGGTGATCGTGATCACCC
ACAGCGGCAGGAAGGGCAGGTACGCCGTGGAGCCCTACCACGGCAAGGTG
GTGGTCCCCGAGGGCCACGCCATCCCCGTGCAGGACTTCCAGGCCCTGAG
CGAGAGCGCCACCATCGTGTATAACGAGAGGGAGTTCGTGAACAGGTACC
TGCACCACATCGCCACCCACGGCGGCGCCCTGAACACCGACGAGGAGTAC
TACAAGACCGTGAAGCCCAGCGAGCACGACGGCGAGTACCTGTACGACAT
CGACAGGAAGCAGTGCGTGAAGAAGGAGCTGGTGACCGGCCTGGGCCTGA
CCGGCGAGCTGGTGGACCCTCCCTTCCACGAGTTCGCCTACGAGAGCCTG
AGGACCAGGCCCGCCGCTCCCTACCAGGTGCCCACCATCGGCGTGTACGG
CGTGCCCGGCAGCGGCAAGAGCGGCATCATCAAGAGCGCCGTGACCAAGA
AGGACCTGGTGGTGAGCGCCAAGAAGGAGAACTGCGCCGAGATCATCAGG
GACGTGAAGAAGATGAAGGGCCTGGACGTGAACGCCAGGACCGTGGACAG
CGTGCTGCTGAACGGCTGCAAGCACCCCGTGGAGACACTGTATATCGACG
AGGCCTTCGCCTGCCACGCCGGCACCCTGAGGGCCCTGATCGCCATCATC
AGGCCCAAGAAGGCCGTGCTGTGCGGCGACCCCAAGCAGTGCGGCTTCTT
CAACATGATGTGCCTGAAGGTGCACTTCAACCACGAGATCTGCACCCAGG
TGTTCCACAAGAGCATCAGCAGGCGGTGCACCAAGAGCGTGACCAGCGTG
GTGAGCACCCTGTTCTACGACAAGAAGATGAGGACCACCAACCCCAAGGA
GACAAAGATCGTGATCGACACCACCGGCAGCACCAAGCCCAAGCAGGACG
ACCTGATCCTGACCTGCTTCAGGGGCTGGGTGAAGCAGCTGCAGATCGAC
TACAAGGGCAACGAGATCATGACCGCCGCCGCTAGCCAGGGCCTGACCAG
GAAGGGCGTGTACGCCGTGAGGTACAAGGTGAACGAGAATCCCCTGTACG
CCCCTACCAGCGAGCACGTGAACGTGCTGCTGACCAGGACCGAGGACAGG
ATCGTGTGGAAGACCCTGGCCGGCGACCCCTGGATCAAGACCCTGACCGC
CAAGTACCCCGGCAACTTCACCGCCACCATCGAGGAGTGGCAGGCCGAGC
ACGACGCCATCATGAGGCACATCCTGGAGAGGCCCGACCCCACCGACGTG
TTCCAGAACAAGGCCAACGTGTGCTGGGCCAAGGCCCTGGTGCCCGTGCT
GAAGACCGCCGGCATCGACATGACCACCGAGCAGTGGAACACCGTGGACT
ACTTCGAGACAGACAAGGCCCACAGCGCCGAGATCGTGCTGAACCAGCTG
TGCGTGAGGTTCTTCGGCCTGGACCTGGACAGCGGCCTGTTCAGCGCCCC
TACCGTGCCCCTGAGCATCAGGAACAACCACTGGGACAACAGCCCCAGCC
CCAACATGTACGGCCTGAACAAGGAGGTGGTGAGGCAGCTGAGCAGGCGG
TACCCTCAGCTGCCCAGGGCCGTGGCCACCGGCAGGGTGTACGACATGAA
CACCGGCACCCTGAGGAACTACGACCCCAGGATCAACCTGGTGCCCGTGA
ACAGGCGGCTGCCCCACGCCCTGGTGCTGCACCACAACGAGCACCCTCAG
AGCGACTTCAGCAGCTTCGTGAGCAAGCTGAAGGGCAGGACCGTGCTGGT
GGTGGGCGAGAAGCTGAGCGTGCCCGGCAAGATGGTGGACTGGCTGAGCG
ACAGGCCCGAGGCCACCTTCCGGGCCAGGCTGGACCTGGGCATCCCCGGC
GACGTGCCCAAGTACGACATCATCTTCGTGAACGTGAGGACCCCTTACAA
GTACCACCACTACCAGCAGTGCGAGGACCACGCCATCAAGCTGAGCATGC
TGACCAAGAAGGCCTGCCTGCACCTGAACCCCGGCGGCACCTGCGTGAGC
ATCGGCTACGGCTACGCCGACAGGGCCAGCGAGAGCATCATCGGCGCCAT
CGCCAGGCTGTTCAAGTTCAGCAGGGTGTGCAAGCCCAAGAGCAGCCTGG
AGGAGACAGAGGTGCTGTTCGTGTTCATCGGCTACGACCGGAAGGCCAGG
ACCCACAACCCCTACAAGCTGAGCAGCACCCTGACCAACATCTACACCGG
CAGCAGGCTGCACGAGGCCGGCTGCGCCCCTAGCTACCACGTGGTGAGGG
GCGACATCGCCACCGCCACCGAGGGCGTGATCATCAACGCCGCCAACAGC
AAGGGCCAGCCCGGCGGCGGGGTGTGCGGCGCCCTGTATAAGAAGTTCCC
CGAGAGCTTCGACCTGCAGCCCATCGAGGTGGGCAAGGCCAGGCTGGTGA
AGGGCGCCGCCAAGCACATCATCCACGCCGTGGGCCCCAACTTCAACAAG
GTGAGCGAGGTGGAGGGCGACAAGCAGCTGGCCGAGGCCTACGAGAGCAT
CGCCAAGATCGTGAACGACAACAACTACAAGAGCGTGGCCATCCCTCTGC
TGAGCACCGGCATCTTCAGCGGCAACAAGGACAGGCTGACCCAGAGCCTG
AACCACCTGCTGACCGCCCTGGACACCACCGACGCCGACGTGGCCATCTA
CTGCAGGGACAAGAAGTGGGAGATGACCCTGAAGGAGGCCGTGGCCAGGC
GGGAGGCCGTGGAGGAGATCTGCATCAGCGACGACAGCAGCGTGACCGAG
CCCGACGCCGAGCTGGTGAGGGTGCACCCCAAGAGCAGCCTGGCCGGCAG
GAAGGGCTACAGCACCAGCGACGGCAAGACCTTCAGCTACCTGGAGGGCA
CCAAGTTCCACCAGGCCGCCAAGGACATCGCCGAGATCAACGCCATGTGG
CCCGTGGCCACCGAGGCCAACGAGCAGGTGTGCATGTATATCCTGGGCGA
GAGCATGAGCAGCATCAGGAGCAAGTGCCCCGTGGAGGAGAGCGAGGCCA
GCACCCCTCCCAGCACCCTGCCCTGCCTGTGCATCCACGCCATGACCCCT
GAGAGGGTGCAGCGGCTGAAGGCCAGCAGGCCCGAGCAGATCACCGTGTG
CAGCAGCTTCCCTCTGCCCAAGTACAGGATCACCGGCGTGCAGAAGATCC
AGTGCAGCCAGCCCATCCTGTTCAGCCCCAAGGTGCCCGCCTACATCCAC
CCCAGGAAGTACCTGGTGGAGACACCCCCCGTGGACGAGACACCCGAGCC
CAGCGCCGAGAACCAGAGCACCGAGGGCACCCCTGAGCAGCCTCCCCTGA
TCACCGAGGACGAGACAAGGACCAGGACCCCTGAGCCCATCATCATTGAG
GAGGAAGAGGAGGACAGCATCAGCCTGCTGAGCGACGGCCCCACCCACCA
GGTGCTGCAGGTGGAGGCCGACATCCACGGCCCTCCCAGCGTGAGCAGCT
CCAGCTGGAGCATCCCTCACGCCAGCGACTTCGACGTGGACAGCCTGAGC
ATCCTGGACACCCTGGAGGGCGCCAGCGTGACCAGCGGCGCCACCAGCGC
CGAGACAAACAGCTACTTCGCCAAGAGCATGGAGTTCCTGGCCAGGCCCG
TGCCCGCCCCTAGGACCGTGTTCAGGAACCCTCCCCACCCCGCCCCTAGG
ACCAGGACCCCTAGCCTGGCCCCTAGCAGGGCCTGCAGCAGGACCAGCCT
GGTGAGCACCCCTCCCGGCGTGAACAGGGTGATCACCAGGGAGGAGCTGG
AGGCCCTGACCCCTAGCAGGACCCCTAGCAGGAGCGTGAGCAGGACCAGC
CTGGTGAGCAACCCTCCCGGCGTGAACAGGGTGATCACCAGGGAGGAGTT
CGAGGCCTTCGTGGCCCAGCAGCAAAGGCGGTTCGACGCCGGCGCCTACA
TCTTCAGCAGCGACACCGGCCAGGGCCACCTGCAGCAGAAGTCCGTGAGG
CAGACCGTGCTGAGCGAGGTGGTGCTGGAGAGGACCGAGCTGGAGATCAG
CTACGCCCCTAGGCTGGACCAGGAGAAGGAGGAGCTGCTGAGGAAGAAGC
TGCAGCTGAACCCCACCCCTGCCAACAGGAGCAGGTACCAGAGCAGGAAG
GTGGAGAACATGAAGGCCATCACCGCCAGGCGGATCCTGCAGGGCCTGGG
CCACTACCTGAAGGCCGAGGGCAAGGTGGAGTGCTACAGGACCCTGCACC
CCGTGCCCCTGTACTCCAGCTCCGTGAACAGGGCCTTCAGCAGCCCCAAG
GTGGCCGTGGAGGCCTGCAACGCCATGCTGAAGGAGAACTTCCCCACCGT
GGCCAGCTACTGCATCATCCCCGAGTACGACGCCTACCTGGACATGGTGG
ACGGCGCCAGCTGCTGCCTGGACACCGCCAGCTTCTGCCCCGCCAAGCTG
AGGAGCTTCCCCAAGAAGCACAGCTACCTGGAGCCCACCATCAGGAGCGC
CGTGCCCAGCGCCATCCAGAACACCCTGCAGAACGTGCTGGCCGCCGCTA
CCAAGAGGAACTGCAACGTGACCCAGATGAGGGAGCTGCCCGTGCTGGAC
AGCGCCGCCTTCAACGTGGAGTGCTTCAAGAAGTACGCCTGCAACAACGA
GTACTGGGAGACATTCAAGGAGAACCCCATCAGGCTGACCGAGGAGAACG
TGGTGAACTACATCACCAAGCTGAAGGGCCCCAAGGCCGCCGCTCTGTTC
GCCAAGACCCACAACCTGAACATGCTGCAGGACATCCCTATGGACAGGTT
CGTGATGGACCTGAAGAGGGACGTGAAGGTGACCCCTGGCACCAAGCACA
CCGAGGAGAGGCCCAAGGTGCAGGTGATCCAGGCCGCCGACCCTCTGGCC
ACCGCCTACCTGTGCGGCATCCACAGGGAGCTGGTGAGGCGGCTGAACGC
CGTGCTGCTGCCCAACATCCACACCCTGTTCGACATGAGCGCCGAGGACT
TCGACGCCATCATCGCCGAGCACTTCCAGCCCGGCGACTGCGTGCTGGAG
ACAGACATCGCCAGCTTCGACAAGAGCGAGGACGACGCTATGGCCCTGAC
CGCCCTGATGATCCTGGAGGACCTGGGCGTGGACGCCGAGCTGCTGACCC
TGATCGAGGCCGCCTTCGGCGAGATCAGCAGCATCCACCTGCCCACCAAG
ACCAAGTTCAAGTTCGGCGCCATGATGAAGTCCGGCATGTTCCTGACCCT
GTTCGTGAACACCGTGATCAACATCGTGATCGCCAGCAGGGTGCTGCGGG
AGAGGCTGACCGGCAGCCCCTGCGCCGCCTTCATCGGCGACGACAACATC
GTGAAGGGCGTGAAGTCCGACAAGCTGATGGCCGACAGGTGCGCCACCTG
GCTGAACATGGAGGTGAAGATCATCGACGCCGTGGTGGGCGAGAAGGCCC
CTTACTTCTGCGGCGGCTTCATCCTGTGCGACAGCGTGACCGGCACCGCC
TGCAGGGTGGCCGACCCTCTGAAGAGGCTGTTCAAGCTGGGCAAGCCCCT
GGCCGCCGACGACGAGCACGACGACGATAGGCGGAGGGCCCTGCACGAGG
AGAGCACCAGGTGGAACAGGGTGGGCATCCTGAGCGAGCTGTGCAAGGCC
GTGGAGAGCAGGTACGAGACAGTGGGCACCAGCATCATCGTGATGGCCAT
GACCACCCTGGCCAGCAGCGTGAAGTCCTTCAGCTACCTGAGG

SEQ ID NO:186 - Self-replicating RNA with codon-optimized nsP1-4 and Luciferase Transgene

atgggcggcgcatgagagaagcccagaccaattacctacccaaaatggag
aaagttcacgttgacatcgaggaagacagcccattcctcagagctttgca
gcggagcttcccgcagtttgaggtagaagccaagcaggtcactgataatg
accatgctaatgccagagcgttttcgcatctggcttcaaaactgatcgaa
acggaggtggacccatccgacacgatccttgacattggaagtgcgcccgc
ccgcagaatgtattctaagcacaagtatcattgtatctgtccgatgagat
gtgcggaagatccggacagattgtataagtatgcaactaagctgaagaaa
aactgtaaggaaataactgataaggaattggacaagaaaatgaaggagct
ggccgccgtcatgagcgaccctgacctggaaactgagactatgtgcctcc
acgacgacgagtcgtgtcgctacgaagggcaagtcgctgtttaccaggat
gtatacGCCGTGGACGGCCCCACCAGCCTGTACCACCAGGCCAACAAGGG
CGTGAGGGTGGCCTACTGGATCGGCTTCGACACCACACCCTTCATGTTCA
AGAACCTGGCCGGCGCCTACCCCAGCTACAGCACCAACTGGGCCGACGAG
ACAGTGCTGACCGCCAGGAACATCGGCCTGTGCAGCAGCGACGTGATGGA
GAGGAGCCGGAGGGGCATGAGCATCCTGAGGAAGAAGTACCTGAAGCCCA
GCAACAACGTGCTGTTCAGCGTGGGCAGCACCATCTACCACGAGAAGAGG
GACCTGCTGAGGAGCTGGCACCTGCCCAGCGTGTTCCACCTGAGGGGCAA
GCAGAACTACACCTGCAGGTGCGAGACAATCGTGAGCTGCGACGGCTACG
TGGTGAAGAGGATCGCCATCAGCCCCGGCCTGTACGGCAAGCCCAGCGGC
TACGCCGCCACCATGCACAGGGAGGGCTTCCTGTGCTGCAAGGTGACCGA
CACCCTGAACGGCGAGAGGGTGAGCTTCCCCGTGTGCACCTACGTGCCCG
CCACCCTGTGCGACCAGATGACCGGCATCCTGGCCACCGACGTGAGCGCC
GACGACGCCCAGAAGCTGCTGGTGGGCCTGAACCAGAGGATCGTGGTGAA
CGGCAGGACCCAGAGGAACACCAACACCATGAAGAACTACCTGCTGCCCG
TGGTGGCCCAGGCCTTCGCCAGGTGGGCCAAGGAGTACAAGGAGGACCAG
GAGGACGAGAGGCCCCTGGGCCTGAGGGACAGGCAGCTGGTGATGGGCTG
CTGCTGGGCCTTCAGGCGGCACAAGATCACCAGCATCTACAAGAGGCCCG
ACACCCAGACCATCATCAAGGTGAACAGCGACTTCCACAGCTTCGTGCTG
CCCAGGATCGGCAGCAACACCCTGGAGATCGGCCTGAGGACCCGGATCAG
GAAGATGCTGGAGGAGCACAAGGAGCCCAGCCCTCTGATCACCGCCGAGG
ACGTGCAGGAGGCCAAGTGCGCCGCCGACGAGGCCAAGGAGGTGAGGGAG
GCCGAGGAGCTGAGGGCCGCCCTGCCTCCCCTGGCCGCCGACGTGGAGGA
GCCCACCCTGGAGGCCGACGTGGACCTGATGCTGCAGGAGGCCGGCGCCG
GCAGCGTGGAGACACCCAGGGGCCTGATCAAGGTGACCAGCTACGACGGC
GAGGACAAGATCGGCAGCTACGCCGTGCTGAGCCCTCAGGCCGTGCTGAA
GTCCGAGAAGCTGAGCTGCATCCACCCTCTGGCCGAGCAGGTGATCGTGA
TCACCCACAGCGGCAGGAAGGGCAGGTACGCCGTGGAGCCCTACCACGGC
AAGGTGGTGGTCCCCGAGGGCCACGCCATCCCCGTGCAGGACTTCCAGGC
CCTGAGCGAGAGCGCCACCATCGTGTATAACGAGAGGGAGTTCGTGAACA
GGTACCTGCACCACATCGCCACCCACGGCGGCGCCCTGAACACCGACGAG
GAGTACTACAAGACCGTGAAGCCCAGCGAGCACGACGGCGAGTACCTGTA
CGACATCGACAGGAAGCAGTGCGTGAAGAAGGAGCTGGTGACCGGCCTGG
GCCTGACCGGCGAGCTGGTGGACCCTCCCTTCCACGAGTTCGCCTACGAG
AGCCTGAGGACCAGGCCCGCCGCTCCCTACCAGGTGCCCACCATCGGCGT
GTACGGCGTGCCCGGCAGCGGCAAGAGCGGCATCATCAAGAGCGCCGTGA
CCAAGAAGGACCTGGTGGTGAGCGCCAAGAAGGAGAACTGCGCCGAGATC
ATCAGGGACGTGAAGAAGATGAAGGGCCTGGACGTGAACGCCAGGACCGT
GGACAGCGTGCTGCTGAACGGCTGCAAGCACCCCGTGGAGACACTGTATA
TCGACGAGGCCTTCGCCTGCCACGCCGGCACCCTGAGGGCCCTGATCGCC
ATCATCAGGCCCAAGAAGGCCGTGCTGTGCGGCGACCCCAAGCAGTGCGG
CTTCTTCAACATGATGTGCCTGAAGGTGCACTTCAACCACGAGATCTGCA
CCCAGGTGTTCCACAAGAGCATCAGCAGGCGGTGCACCAAGAGCGTGACC
AGCGTGGTGAGCACCCTGTTCTACGACAAGAAGATGAGGACCACCAACCC
CAAGGAGACAAAGATCGTGATCGACACCACCGGCAGCACCAAGCCCAAGC
AGGACGACCTGATCCTGACCTGCTTCAGGGGCTGGGTGAAGCAGCTGCAG
ATCGACTACAAGGGCAACGAGATCATGACCGCCGCCGCTAGCCAGGGCCT
GACCAGGAAGGGCGTGTACGCCGTGAGGTACAAGGTGAACGAGAATCCCC
TGTACGCCCCTACCAGCGAGCACGTGAACGTGCTGCTGACCAGGACCGAG
GACAGGATCGTGTGGAAGACCCTGGCCGGCGACCCCTGGATCAAGACCCT
GACCGCCAAGTACCCCGGCAACTTCACCGCCACCATCGAGGAGTGGCAGG
CCGAGCACGACGCCATCATGAGGCACATCCTGGAGAGGCCCGACCCCACC
GACGTGTTCCAGAACAAGGCCAACGTGTGCTGGGCCAAGGCCCTGGTGCC
CGTGCTGAAGACCGCCGGCATCGACATGACCACCGAGCAGTGGAACACCG
TGGACTACTTCGAGACAGACAAGGCCCACAGCGCCGAGATCGTGCTGAAC
CAGCTGTGCGTGAGGTTCTTCGGCCTGGACCTGGACAGCGGCCTGTTCAG
CGCCCCTACCGTGCCCCTGAGCATCAGGAACAACCACTGGGACAACAGCC
CCAGCCCCAACATGTACGGCCTGAACAAGGAGGTGGTGAGGCAGCTGAGC
AGGCGGTACCCTCAGCTGCCCAGGGCCGTGGCCACCGGCAGGGTGTACGA
CATGAACACCGGCACCCTGAGGAACTACGACCCCAGGATCAACCTGGTGC
CCGTGAACAGGCGGCTGCCCCACGCCCTGGTGCTGCACCACAACGAGCAC
CCTCAGAGCGACTTCAGCAGCTTCGTGAGCAAGCTGAAGGGCAGGACCGT
GCTGGTGGTGGGCGAGAAGCTGAGCGTGCCCGGCAAGATGGTGGACTGGC
TGAGCGACAGGCCCGAGGCCACCTTCCGGGCCAGGCTGGACCTGGGCATC
CCCGGCGACGTGCCCAAGTACGACATCATCTTCGTGAACGTGAGGACCCC
TTACAAGTACCACCACTACCAGCAGTGCGAGGACCACGCCATCAAGCTGA
GCATGCTGACCAAGAAGGCCTGCCTGCACCTGAACCCCGGCGGCACCTGC
GTGAGCATCGGCTACGGCTACGCCGACAGGGCCAGCGAGAGCATCATCGG
CGCCATCGCCAGGCTGTTCAAGTTCAGCAGGGTGTGCAAGCCCAAGAGCA
GCCTGGAGGAGACAGAGGTGCTGTTCGTGTTCATCGGCTACGACCGGAAG
GCCAGGACCCACAACCCCTACAAGCTGAGCAGCACCCTGACCAACATCTA
CACCGGCAGCAGGCTGCACGAGGCCGGCTGCGCCCCTAGCTACCACGTGG
TGAGGGGCGACATCGCCACCGCCACCGAGGGCGTGATCATCAACGCCGCC
AACAGCAAGGGCCAGCCCGGCGGCGGGGTGTGCGGCGCCCTGTATAAGAA
GTTCCCCGAGAGCTTCGACCTGCAGCCCATCGAGGTGGGCAAGGCCAGGC
TGGTGAAGGGCGCCGCCAAGCACATCATCCACGCCGTGGGCCCCAACTTC
AACAAGGTGAGCGAGGTGGAGGGCGACAAGCAGCTGGCCGAGGCCTACGA
GAGCATCGCCAAGATCGTGAACGACAACAACTACAAGAGCGTGGCCATCC
CTCTGCTGAGCACCGGCATCTTCAGCGGCAACAAGGACAGGCTGACCCAG
AGCCTGAACCACCTGCTGACCGCCCTGGACACCACCGACGCCGACGTGGC
CATCTACTGCAGGGACAAGAAGTGGGAGATGACCCTGAAGGAGGCCGTGG
CCAGGCGGGAGGCCGTGGAGGAGATCTGCATCAGCGACGACAGCAGCGTG
ACCGAGCCCGACGCCGAGCTGGTGAGGGTGCACCCCAAGAGCAGCCTGGC
CGGCAGGAAGGGCTACAGCACCAGCGACGGCAAGACCTTCAGCTACCTGG
AGGGCACCAAGTTCCACCAGGCCGCCAAGGACATCGCCGAGATCAACGCC
ATGTGGCCCGTGGCCACCGAGGCCAACGAGCAGGTGTGCATGTATATCCT
GGGCGAGAGCATGAGCAGCATCAGGAGCAAGTGCCCCGTGGAGGAGAGCG
AGGCCAGCACCCCTCCCAGCACCCTGCCCTGCCTGTGCATCCACGCCATG
ACCCCTGAGAGGGTGCAGCGGCTGAAGGCCAGCAGGCCCGAGCAGATCAC
CGTGTGCAGCAGCTTCCCTCTGCCCAAGTACAGGATCACCGGCGTGCAGA
AGATCCAGTGCAGCCAGCCCATCCTGTTCAGCCCCAAGGTGCCCGCCTAC
ATCCACCCCAGGAAGTACCTGGTGGAGACACCCCCCGTGGACGAGACACC
CGAGCCCAGCGCCGAGAACCAGAGCACCGAGGGCACCCCTGAGCAGCCTC
CCCTGATCACCGAGGACGAGACAAGGACCAGGACCCCTGAGCCCATCATC
ATTGAGGAGGAAGAGGAGGACAGCATCAGCCTGCTGAGCGACGGCCCCAC
CCACCAGGTGCTGCAGGTGGAGGCCGACATCCACGGCCCTCCCAGCGTGA
GCAGCTCCAGCTGGAGCATCCCTCACGCCAGCGACTTCGACGTGGACAGC
CTGAGCATCCTGGACACCCTGGAGGGCGCCAGCGTGACCAGCGGCGCCAC
CAGCGCCGAGACAAACAGCTACTTCGCCAAGAGCATGGAGTTCCTGGCCA
GGCCCGTGCCCGCCCCTAGGACCGTGTTCAGGAACCCTCCCCACCCCGCC
CCTAGGACCAGGACCCCTAGCCTGGCCCCTAGCAGGGCCTGCAGCAGGAC
CAGCCTGGTGAGCACCCCTCCCGGCGTGAACAGGGTGATCACCAGGGAGG
AGCTGGAGGCCCTGACCCCTAGCAGGACCCCTAGCAGGAGCGTGAGCAGG
ACCAGCCTGGTGAGCAACCCTCCCGGCGTGAACAGGGTGATCACCAGGGA
GGAGTTCGAGGCCTTCGTGGCCCAGCAGCAAAGGCGGTTCGACGCCGGCG
CCTACATCTTCAGCAGCGACACCGGCCAGGGCCACCTGCAGCAGAAGTCC
GTGAGGCAGACCGTGCTGAGCGAGGTGGTGCTGGAGAGGACCGAGCTGGA
GATCAGCTACGCCCCTAGGCTGGACCAGGAGAAGGAGGAGCTGCTGAGGA
AGAAGCTGCAGCTGAACCCCACCCCTGCCAACAGGAGCAGGTACCAGAGC
AGGAAGGTGGAGAACATGAAGGCCATCACCGCCAGGCGGATCCTGCAGGG
CCTGGGCCACTACCTGAAGGCCGAGGGCAAGGTGGAGTGCTACAGGACCC
TGCACCCCGTGCCCCTGTACTCCAGCTCCGTGAACAGGGCCTTCAGCAGC
CCCAAGGTGGCCGTGGAGGCCTGCAACGCCATGCTGAAGGAGAACTTCCC
CACCGTGGCCAGCTACTGCATCATCCCCGAGTACGACGCCTACCTGGACA
TGGTGGACGGCGCCAGCTGCTGCCTGGACACCGCCAGCTTCTGCCCCGCC
AAGCTGAGGAGCTTCCCCAAGAAGCACAGCTACCTGGAGCCCACCATCAG
GAGCGCCGTGCCCAGCGCCATCCAGAACACCCTGCAGAACGTGCTGGCCG
CCGCTACCAAGAGGAACTGCAACGTGACCCAGATGAGGGAGCTGCCCGTG
CTGGACAGCGCCGCCTTCAACGTGGAGTGCTTCAAGAAGTACGCCTGCAA
CAACGAGTACTGGGAGACATTCAAGGAGAACCCCATCAGGCTGACCGAGG
AGAACGTGGTGAACTACATCACCAAGCTGAAGGGCCCCAAGGCCGCCGCT
CTGTTCGCCAAGACCCACAACCTGAACATGCTGCAGGACATCCCTATGGA
CAGGTTCGTGATGGACCTGAAGAGGGACGTGAAGGTGACCCCTGGCACCA
AGCACACCGAGGAGAGGCCCAAGGTGCAGGTGATCCAGGCCGCCGACCCT
CTGGCCACCGCCTACCTGTGCGGCATCCACAGGGAGCTGGTGAGGCGGCT
GAACGCCGTGCTGCTGCCCAACATCCACACCCTGTTCGACATGAGCGCCG
AGGACTTCGACGCCATCATCGCCGAGCACTTCCAGCCCGGCGACTGCGTG
CTGGAGACAGACATCGCCAGCTTCGACAAGAGCGAGGACGACGCTATGGC
CCTGACCGCCCTGATGATCCTGGAGGACCTGGGCGTGGACGCCGAGCTGC
TGACCCTGATCGAGGCCGCCTTCGGCGAGATCAGCAGCATCCACCTGCCC
ACCAAGACCAAGTTCAAGTTCGGCGCCATGATGAAGTCCGGCATGTTCCT
GACCCTGTTCGTGAACACCGTGATCAACATCGTGATCGCCAGCAGGGTGC
TGCGGGAGAGGCTGACCGGCAGCCCCTGCGCCGCCTTCATCGGCGACGAC
AACATCGTGAAGGGCGTGAAGTCCGACAAGCTGATGGCCGACAGGTGCGC
CACCTGGCTGAACATGGAGGTGAAGATCATCGACGCCGTGGTGGGCGAGA
AGGCCCCTTACTTCTGCGGCGGCTTCATCCTGTGCGACAGCGTGACCGGC
ACCGCCTGCAGGGTGGCCGACCCTCTGAAGAGGCTGTTCAAGCTGGGCAA
GCCCCTGGCCGCCGACGACGAGCACGACGACGATAGGCGGAGGGCCCTGC
ACGAGGAGAGCACCAGGTGGAACAGGGTGGGCATCCTGAGCGAGCTGTGC
AAGGCCGTGGAGAGCAGGTACGAGACAGTGGGCACCAGCATCATCGTGAT
GGCCATGACCACCCTGGCCAGCAGCGTGAAGTCCTTCAGCTACCTGAGGG
GGGCCCCTATAACTCTCTACGGCTAACCTGAATGGACTACGACATAGTCT
AGTCCGCCAAGGCCGCCACCATGGAAGATGCCAAAAACATTAAGAAGGGC
CCAGCGCCATTCTACCCACTCGAAGACGGGACCGCCGGCGAGCAGCTGCA
CAAAGCCATGAAGCGCTACGCCCTGGTGCCCGGCACCATCGCCTTTACCG
ACGCACATATCGAGGTGGACATTACCTACGCCGAGTACTTCGAGATGAGC
GTTCGGCTGGCAGAAGCTATGAAGCGCTATGGGCTGAATACAAACCATCG
GATCGTGGTGTGCAGCGAGAATAGCTTGCAGTTCTTCATGCCCGTGTTGG
GTGCCCTGTTCATCGGTGTGGCTGTGGCCCCAGCTAACGACATCTACAAC
GAGCGCGAGCTGCTGAACAGCATGGGCATCAGCCAGCCCACCGTCGTATT
CGTGAGCAAGAAAGGGCTGCAAAAGATCCTCAACGTGCAAAAGAAGCTAC
CGATCATACAAAAGATCATCATCATGGATAGCAAGACCGACTACCAGGGC
TTCCAAAGCATGTACACCTTCGTGACTTCCCATTTGCCACCCGGCTTCAA
CGAGTACGACTTCGTGCCCGAGAGCTTCGACCGGGACAAAACCATCGCCC
TGATCATGAACAGTAGTGGCAGTACCGGATTGCCCAAGGGCGTAGCCCTA
CCGCACCGCACCGCTTGTGTCCGATTCAGTCATGCCCGCGACCCCATCTT
CGGCAACCAGATCATCCCCGACACCGCTATCCTCAGCGTGGTGCCATTTC
ACCACGGCTTCGGCATGTTCACCACGCTGGGCTACTTGATCTGCGGCTTT
CGGGTCGTGCTCATGTACCGCTTCGAGGAGGAGCTATTCTTGCGCAGCTT
GCAAGACTATAAGATTCAATCTGCCCTGCTGGTGCCCACACTATTTAGCT
TCTTCGCTAAGAGCACTCTCATCGACAAGTACGACCTAAGCAACTTGCAC
GAGATCGCCAGCGGCGGGGCGCCGCTCAGCAAGGAGGTAGGTGAGGCCGT
GGCCAAACGCTTCCACCTACCAGGCATCCGACAGGGCTACGGCCTGACAG
AAACAACCAGCGCCATTCTGATCACCCCCGAAGGGGACGACAAGCCTGGC
GCAGTAGGCAAGGTGGTGCCCTTCTTCGAGGCTAAGGTGGTGGACTTGGA
CACCGGTAAGACACTGGGTGTGAACCAGCGCGGCGAGCTGTGCGTCCGTG
GCCCCATGATCATGAGCGGCTACGTTAACAACCCCGAGGCTACAAACGCT
CTCATCGACAAGGACGGCTGGCTGCACAGCGGCGACATCGCCTACTGGGA
CGAGGACGAGCACTTCTTCATCGTGGACCGGCTGAAGTCCCTGATCAAAT
ACAAGGGCTACCAGGTAGCCCCAGCCGAACTGGAGAGCATCCTGCTGCAA
CACCCCAACATCTTCGACGCCGGGGTCGCCGGCCTGCCCGACGACGATGC
CGGCGAGCTGCCCGCCGCAGTCGTCGTGCTGGAACACGGTAAAACCATGA
CCGAGAAGGAGATCGTGGACTATGTGGCCAGCCAGGTTACAACCGCCAAG
AAGCTGCGCGGTGGTGTTGTGTTCGTGGACGAGGTGCCTAAAGGACTGAC
CGGCAAGTTGGACGCCCGCAAGATCCGCGAGATTCTCATTAAGGCCAAGA
AGGGCGGCAAGATCGCCGTGTAACTCGAGTATGTTACGTGCAAAGGTGAT
TGTCACCCCCCGAAAGACCATATTGTGACACACCCTCAGTATCACGCCCA
AACATTTACAGCCGCGGTGTCAAAAACCGCGTGGACGTGGTTAACATCCC
TGCTGGGAGGATCAGCCGTAATTATTATAATTGGCTTGGTGCTGGCTACT
ATTGTGGCCATGTACGTGCTGACCAACCAGAAACATAATTGAATACAGCA
GCAATTGGCAAGCTGCTTACATAGAACTCGCGGCGATTGGCATGCCGCCT
TAAAATTTTTATTTTATTTTTTCTTTTCTTTTCCGAATCGGATTTTGTTT
TTAATATTTCAAAAAAAAAAAAAAAAAAAAAAAAATctagAAAAAAAAAA
AAAAAAAAAAAAAAAAAAAAAAAAAAAAAAAAAAAAAAAAAAAAAAAAAA
AAAAAAAAAAAAAAAAAAAAaaaaaaaaaaaaaaaaaaaa

SEQ ID NO:187 - nsP1-4 amino acid sequence (encoded by SEQ ID NO:6 and SEQ ID NO:42)

MEKVHVDIEEDSPFLRALQRSFPQFEVEAKQVTDNDHANARAFSHLASKL
IETEVDPSDTILDIGSAPARRMYSKHKYHCICPMRCAEDPDRLYKYATKL
KKNCKEITDKELDKKMKELAAVMSDPDLETETMCLHDDESCRYEGQVAVY
QDVYAVDGPTSLYHQANKGVRVAYWIGFDTTPFMFKNLAGAYPSYSTNWA
DETVLTARNIGLCSSDVMERSRRGMSILRKKYLKPSNNVLFSVGSTIYHE
KRDLLRSWHLPSVFHLRGKQNYTCRCETIVSCDGYVVKRIAISPGLYGKP
SGYAATMHREGFLCCKVTDTLNGERVSFPVCTYVPATLCDQMTGILATDV
SADDAQKLLVGLNQRIVVNGRTQRNTNTMKNYLLPVVAQAFARWAKEYKE
DQEDERPLGLRDRQLVMGCCWAFRRHKITSIYKRPDTQTIIKVNSDFHSF
VLPRIGSNTLEIGLRTRIRKMLEEHKEPSPLITAEDVQEAKCAADEAKEV
REAEELRAALPPLAADVEEPTLEADVDLMLQEAGAGSVETPRGLIKVTSY
DGEDKIGSYAVLSPQAVLKSEKLSCIHPLAEQVIVITHSGRKGRYAVEPY
HGKVVVPEGHAIPVQDFQALSESATIVYNEREFVNRYLHHIATHGGALNT
DEEYYKTVKPSEHDGEYLYDIDRKQCVKKELVTGLGLTGELVDPPFHEFA
YESLRTRPAAPYQVPTIGVYGVPGSGKSGIIKSAVTKKDLVVSAKKENCA
EIIRDVKKMKGLDVNARTVDSVLLNGCKHPVETLYIDEAFACHAGTLRAL
IAIIRPKKAVLCGDPKQCGFFNMMCLKVHFNHEICTQVFHKSISRRCTKS
VTSVVSTLFYDKKMRTTNPKETKIVIDTTGSTKPKQDDLILTCFRGWVKQ
LQIDYKGNEIMTAAASQGLTRKGVYAVRYKVNENPLYAPTSEHVNVLLTR
TEDRIVWKTLAGDPWIKTLTAKYPGNFTATIEEWQAEHDAIMRHILERPD
PTDVFQNKANVCWAKALVPVLKTAGIDMTTEQWNTVDYFETDKAHSAEIV
LNQLCVRFFGLDLDSGLFSAPTVPLSIRNNHWDNSPSPNMYGLNKEVVRQ
LSRRYPQLPRAVATGRVYDMNTGTLRNYDPRINLVPVNRRLPHALVLHHN
EHPQSDFSSFVSKLKGRTVLVVGEKLSVPGKMVDWLSDRPEATFRARLDL
GIPGDVPKYDIIFVNVRTPYKYHHYQQCEDHAIKLSMLTKKACLHLNPGG
TCVSIGYGYADRASESIIGAIARLFKFSRVCKPKSSLEETEVLFVFIGYD
RKARTHNPYKLSSTLTNIYTGSRLHEAGCAPSYHVVRGDIATATEGVIIN
AANSKGQPGGGVCGALYKKFPESFDLQPIEVGKARLVKGAAKHIIHAVGP
NFNKVSEVEGDKQLAEAYESIAKIVNDNNYKSVAIPLLSTGIFSGNKDRL
TQSLNHLLTALDTTDADVAIYCRDKKWEMTLKEAVARREAVEEICISDDS
SVTEPDAELVRVHPKSSLAGRKGYSTSDGKTFSYLEGTKFHQAAKDIAEI
NAMWPVATEANEQVCMYILGESMSSIRSKCPVEESEASTPPSTLPCLCIH
AMTPERVQRLKASRPEQITVCSSFPLPKYRITGVQKIQCSQPILFSPKVP
AYIHPRKYLVETPPVDETPEPSAENQSTEGTPEQPPLITEDETRTRTPEP
IIIEEEEEDSISLLSDGPTHQVLQVEADIHGPPSVSSSSWSIPHASDFDV
DSLSILDTLEGASVTSGATSAETNSYFAKSMEFLARPVPAPRTVFRNPPH
PAPRTRTPSLAPSRACSRTSLVSTPPGVNRVITREELEALTPSRTPSRSV
SRTSLVSNPPGVNRVITREEFEAFVAQQQRRFDAGAYIFSSDTGQGHLQQ
KSVRQTVLSEVVLERTELEISYAPRLDQEKEELLRKKLQLNPTPANRSRY
QSRKVENMKAITARRILQGLGHYLKAEGKVECYRTLHPVPLYSSSVNRAF
SSPKVAVEACNAMLKENFPTVASYCIIPEYDAYLDMVDGASCCLDTASFC
PAKLRSFPKKHSYLEPTIRSAVPSAIQNTLQNVLAAATKRNCNVTQMREL
PVLDSAAFNVECFKKYACNNEYWETFKENPIRLTEENVVNYITKLKGPKA
AALFAKTHNLNMLQDIPMDRFVMDLKRDVKVTPGTKHTEERPKVQVIQAA
DPLATAYLCGIHRELVRRLNAVLLPNIHTLFDMSAEDFDAIIAEHFQPGD
CVLETDIASFDKSEDDAMALTALMILEDLGVDAELLTLIEAAFGEISSIH
LPTKTKFKFGAMMKSGMFLTLFVNTVINIVIASRVLRERLTGSPCAAFIG
DDNIVKGVKSDKLMADRCATWLNMEVKIIDAVVGEKAPYFCGGFILCDSV
TGTACRVADPLKRLFKLGKPLAADDEHDDDRRRALHEESTRWNRVGILSE
LCKAVESRYETVGTSIIVMAMTTLASSVKSFSYLRGAPITLYG

SEQ ID NO:188 - nsP1-4 amino acid sequence (encoded by SEQ ID NO:20)

MEKVHVDIEEDSPFLRALQRSFPQFEVEAKQVTDNDHANARAFSHLASKL
IETEVDPSDTILDIGSAPARRMYSKHKYHCICPMRCAEDPDRLYKYATKL
KKNCKEITDKELDKKMKELAAVMSDPDLETETMCLHDDESCRYEGQVAVY
QDVYAVDGPTSLYHQANKGVRVAYWIGFDTTPFMFKNLAGAYPSYSTNWA
DETVLTARNIGLCSSDVMERSRRGMSILRKKYLKPSNNVLFSVGSTIYHE
KRDLLRSWHLPSVFHLRGKQNYTCRCETIVSCDGYVVKRIAISPGLYGKP
SGYAATMHREGFLCCKVTDTLNGERVSFPVCTYVPATLCDQMTGILATDV
SADDAQKLLVGLNQRIVVNGRTQRNTNTMKNYLLPVVAQAFARWAKEYKE
DQEDERPLGLRDRQLVMGCCWAFRRHKITSIYKRPDTQTIIKVNSDFHSF
VLPRIGSNTLEIGLRTRIRKMLEEHKEPSPLITAEDVQEAKCAADEAKEV
REAEELRAALPPLAADVEEPTLEADVDLMLQEAGAGSVETPRGLIKVTSY
DGEDKIGSYAVLSPQAVLKSEKLSCIHPLAEQVIVITHSGRKGRYAVEPY
HGKVVVPEGHAIPVQDFQALSESATIVYNEREFVNRYLHHIATHGGALNT
DEEYYKTVKPSEHDGEYLYDIDRKQCVKKELVTGLGLTGELVDPPFHEFA
YESLRTRPAAPYQVPTIGVYGVPGSGKSGIIKSAVTKKDLVVSAKKENCA
EIIRDVKKMKGLDVNARTVDSVLLNGCKHPVETLYIDEAFACHAGTLRAL
IAIIRPKKAVLCGDPKQCGFFNMMCLKVHFNHEICTQVFHKSISRRCTKS
VTSVVSTLFYDKKMRTTNPKETKIVIDTTGSTKPKQDDLILTCFRGWVKQ
LQIDYKGNEIMTAAASQGLTRKGVYAVRYKVNENPLYAPTSEHVNVLLTR
TEDRIVWKTLAGDPWIKTLTAKYPGNFTATIEEWQAEHDAIMRHILERPD
PTDVFQNKANVCWAKALVPVLKTAGIDMTTEQWNTVDYFETDKAHSAEIV
LNQLCVRFFGLDLDSGLFSAPTVPLSIRNNHWDNSPSPNMYGLNKEVVRQ
LSRRYPQLPRAVATGRVYDMNTGTLRNYDPRINLVPVNRRLPHALVLHHN
EHPQSDFSSFVSKLKGRTVLVVGEKLSVPGKMVDWLSDRPEATFRARLDL
GIPGDVPKYDIIFVNVRTPYKYHHYQQCEDHAIKLSMLTKKACLHLNPGG
TCVSIGYGYADRASESIIGAIARLFKFSRVCKPKSSLEETEVLFVFIGYD
RKARTHNPYKLSSTLTNIYTGSRLHEAGCAPSYHVVRGDIATATEGVIIN
AANSKGQPGGGVCGALYKKFPESFDLQPIEVGKARLVKGAAKHIIHAVGP
NFNKVSEVEGDKQLAEAYESIAKIVNDNNYKSVAIPLLSTGIFSGNKDRL
TQSLNHLLTALDTTDADVAIYCRDKKWEMTLKEAVARREAVEEICISDDS
SVTEPDAELVRVHPKSSLAGRKGYSTSDGKTFSYLEGTKFHQAAKDIAEI
NAMWPVATEANEQVCMYILGESMSSIRSKCPVEESEASTPPSTLPCLCIH
AMTPERVQRLKASRPEQITVCSSFPLPKYRITGVQKIQCSQPILFSPKVP
AYIHPRKYLVETPPVDETPEPSAENQSTEGTPEQPPLITEDETRTRTPEP
IIIEEEEEDSISLLSDGPTHQVLQVEADIHGPPSVSSSSWSIPHASDFDV
DSLSILDTLEGASVTSGATSAETNSYFAKSMEFLARPVPAPRTVFRNPPH
PAPRTRTPSLAPSRACSRTSLVSTPPGVNRVITREELEALTPSRTPSRSV
SRTSLVSNPPGVNRVITREEFEAFVAQQQRRFDAGAYIFSSDTGQGHLQQ
KSVRQTVLSEVVLERTELEISYAPRLDQEKEELLRKKLQLNPTPANRSRY
QSRKVENMKAITARRILQGLGHYLKAEGKVECYRTLHPVPLYSSSVNRAF
SSPKVAVEACNAMLKENFPTVASYCIIPEYDAYLDMVDGASCCLDTASFC
PAKLRSFPKKHSYLEPTIRSAVPSAIQNTLQNVLAAATKRNCNVTQMREL
PVLDSAAFNVECFKKYACNNEYWETFKENPIRLTEENVVNYITKLKGPKA
AALFAKTHNLNMLQDIPMDRFVMDLKRDVKVTPGTKHTEERPKVQVIQAA
DPLATAYLCGIHRELVRRLNAVLLPNIHTLFDMSAEDFDAIIAEHFQPGD
CVLETDIASFDKSEDDAMALTALMILEDLGVDAELLTLIEAAFGEISSIH
LPTKTKFKFGAMMKSGMFLTLFVNTVINIVIASRVLRERLTGSPCAAFIG
DDNIVKGVKSDKLMADRCATWLNMEVKIIDAVVGEKAPYFCGGFILCDSV
TGTACRVADPLKRLFKLGKPLAADDEHDDDRRRALHEESTRWNRVGILSE
LCKAVESRYETVGTSIIVMAMTTLASSVKSFSYLRGAPITLYG*

SEQ ID NO:189 - TEV (5′ UTR)

UCAACACAACAUAUACAAAACAAACGAAUCUCAAGCAAUCAAGCAUUCUA
CUUCUAUUGCAGCAAUUUAAAUCAUUUCUUUUAAAGCAAAAGCAAUUUUC
UGAAAAUUUUCACCAUUUACGAACGAUAG

SEQ ID NO:190 - AT1G58420 (5′ UTR)

AUUAUUACAUCAAAACAAAAAGCCGCCA

SEQ ID NO: 191 - ARC5-2 (5′ UTR)

CUUAAGGGGGCGCUGCCUACGGAGGUGGCAGCCAUCUCCUUCUCGGCAUC
AAGCUUACCAUGGUGCCCCAGGCCCUGCUCUUGGUCCCGCUGCUGGUGUU
CCCCCUCUGCUUCGGCAAGUUCCCCAUCUACACCAUCCCCGACAAGCUGG
GGCCGUGGAGCCCCAUCGACAUCCACCACCUGUCCUGCCCCAACAACCUC
GUGGUCGAGGACGAGGGCUGCACCAACCUGAGCGGGUUCUCCUAC

SEQ ID NO:192 - HCV (5′ UTR)

UGAGUGUCGU ACAGCCUCCA GGCCCCCCCC UCCCGGGAGA GCCAUAGUGG
UCUGCGGAACCGGUGAGUAC ACCGGAAUUG CCGGGAAGAC UGGGUCCUUU
CUUGGAUAAA CCCACUCUAUGCCCGGCCAU UUGGGCGUGC CCCCGCAAGA
CUGCUAGCCG AGUAGUGUUG GGUUGCG

SEQ ID NO:193 - HUMAN ALBUMIN (5′ UTR)

AAUUAUUGGUUAAAGAAGUAUAUUAGUGCUAAUUUCCCUCCGUUUGUCCU
AGCUUUUCUCUUCUGUCAACCCCACACGCCUUUGGCACA

SEQ ID NO:194 - EMCV (5′ UTR)

CUCCCUCCCC CCCCCCUAAC GUUACUGGCC GAAGCCGCUU GGAAUAAGGC
CGGUGUGCGU UUGUCUAUAU GUUAUUUUCC ACCAUAUUGC CGUCUUUUGG
CAAUGUGAGG GCCCGGAAAC CUGGCCCUGU CUUCUUGACG AGCAUUCCUA
GGGGUCUUUC CCCUCUCGCC AAAGGAAUGC AAGGUCUGUU GAAUGUCGUG
AAGGAAGCAG UUCCUCUGGA AGCUUCUUGA AGACAAACAA CGUCUGUAGC
GACCCUUUGC AGGCAGCGGA ACCCCCCACC UGGCGACAGG UGCCUCUGCG
GCCAAAAGCC ACGUGUAUAA GAUACACCUG CAAAGGCGGC ACAACCCCAG
UGCCACGUUG UGAGUUGGAU AGUUGUGGAA AGAGUCAAAU GGCUCUCCUC
AAGCGUAUUC AACAAGGGGC UGAAGGAUGC CCAGAAGGUA CCCCAUUGUA
UGGGAUCUGA UCUGGGGCCU CGGUGCACAU GCUUUACGUG UGUUUAGUCG
AGGUUAAAAA ACGUCUAGGC CCCCCGAACC ACGGGGACGU GGUUUUCCUU
UGAAAAACAC GAUGAUAAU

SEQ ID NO:195 - AT1G67090 (5′ UTR)

CACAAAGAGUAAAGAAGAACA

SEQ ID NO:196 - AT1G35720 (5′ UTR)

AACACUAAAAGUAGAAGAAAA

SEQ ID NO:197 - AT5G45900 (5′ UTR)

CUCAGAAAGAUAAGAUCAGCC

SEQ ID NO:198 - AT5G61250 (5′ UTR)

AACCAAUCGAAAGAAACCAAA

SEQ ID NO:199 - AT5G46430 (5′ UTR)

CUCUAAUCACCAGGAGUAAAA

SEQ ID NO:200 - AT5G47110 (5′ UTR)

GAGAGAGAUCUUAACAAAAAA

SEQ ID NO:201 - AT1G03110 (5′ UTR)

UGUGUAACAACAACAACAACA

SEQ ID NO:202 - AT3G12380 (5′ UTR)

CCGCAGUAGGAAGAGAAAGCC

SEQ ID NO:203 - AT5G45910 (5′ UTR)

AAAAAAAAAAGAAAUCAUAAA

SEQ ID NO:204 - AT1G07260 (5′ UTR)

GAGAGAAGAAAGAAGAAGACG

SEQ ID NO:205 - AT3G55500 (5′ UTR)

CAAUUAAAAAUACUUACCAAA

SEQ ID NO:206 - AT3G46230 (5′ UTR)

GCAAACAGAGUAAGCGAAACG

SEQ ID NO:207 - AT2G36170 (5′ UTR)

GCGAAGAAGACGAACGCAAAG

SEQ ID NO:208 - AT1G10660 (5′ UTR)

UUAGGACUGUAUUGACUGGCC

SEQ ID NO:209 - AT4G14340 (5′ UTR)

AUCAUCGGAAUUCGGAAAAAG

SEQ ID NO:210 - AT1G49310 (5′ UTR)

AAAACAAAAGUUAAAGCAGAC

SEQ ID NO:211 - AT4G14360 (5′ UTR)

UUUAUCUCAAAUAAGAAGGCA

SEQ ID NO:212 - AT1G28520 (5′ UTR)

GGUGGGGAGGUGAGAUUUCUU

SEQ ID NO:213 - AT1G20160 (5′ UTR)

UGAUUAGGAAACUACAAAGCC

SEQ ID NO:214 - AT5G37370 (5′ UTR)

CAUUUUUCAAUUUCAUAAAAC

SEQ ID NO:215 - AT4G11320 (5′ UTR)

UUACUUUUAAGCCCAACAAAA

SEQ ID NO:216 - AT5G40850 (5′ UTR)

GGCGUGUGUGUGUGUUGUUGA

SEQ ID NO:217 - AT1G06150 (5′ UTR)

GUGGUGAAGGGGAAGGUUUAG

SEQ ID NO:218 - AT2G26080 (5′ UTR)

UUGUUUUUUUUUGGUUUGGUU

SEQ ID NO:219 - XBG (3′ UTR)

CUAGUGACUGACUAGGAUCUGGUUACCACUAAACCAGCCUCAAGAACACC
CGAAUGGAGUCUCUAAGCUACAUAAUACCAACUUACACUUACAAAAUGUU
GUCCCCCAAAAUGUAGCCAUUCGUAUCUGCUCCUAAUAAAAAGAAAGUUU
CUUCACAU

SEQ ID NO:220 - HUMAN HAPTOGLOBIN (3′ UTR)

UGCAAGGCUGGCCGGAAGCCCUUGCCUGAAAGCAAGAUUUCAGCCUGGAA
GAGGGCAAAGUGGACGGGAGUGGACAGGAGUGGAUGCGAUAAGAUGUGGU
UUGAAGCUGAUGGGUGCCAGCCCUGCAUUGCUGAGUCAAUCAAUAAAGAG
CUUUCUUUUGACCCAU

SEQ ID NO:221 - HUMAN APOLIPOPROTEIN E (3′ UTR)

ACGCCGAAGCCUGCAGCCAUGCGACCCCACGCCACCCCGUGCCUCCUGCC
UCCGCGCAGCCUGCAGCGGGAGACCCUGUCCCCGCCCCAGCCGUCCUCCU
GGGGUGGACCCUAGUUUAAUAAAGAUUCACCAAGUUUCACGCA

SEQ ID NO:222 - HCV (3′ UTR)

UAGAGCGGCAAACCCUAGCUACACUCCAUAGCUAGUUUCUUUUUUUUUUG
UUUUUUUUUUUUUUUUUUUUUUUUUUUUUUUUUUUUUUUCCUUUCUUUUC
CUUCUUUUUUUCCUCUUUUCUUGGUGGCUCCAUCUUAGCCCUAGUCACGG
CUAGCUGUGAAAGGUCCGUGAGCCGCAUGACUGCAGAGAGUGCCGUAACU
GGUCUCUCUGCAGAUCAUGU

SEQ ID NO:223 - MOUSE ALBUMIN (3′ UTR)

ACACAUCACAACCACAACCUUCUCAGGCUACCCUGAGAAAAAAAGACAUG
AAGACUCAGGACUCAUCUUUUCUGUUGGUGUAAAAUCAACACCCUAAGGA
ACACAAAUUUCUUUAAACAUUUGACUUCUUGUCUCUGUGCUGCAAUUAAU
AAAAAAUGGAAAGAAUCUAC

SEQ ID NO:224 - HUMAN ALPHA GLOBIN (3′ UTR)

GCUGGAGCCUCGGUAGCCGUUCCUCCUGCCCGCUGGGCCUCCCAACGGGC
CCUCCUCCCCUCCUUGCACCGGCCCUUCCUGGUCUUUGAAUAAAGUCUGA
GUGGGCAGCA

SEQ ID NO:225 - EMCV (3′ UTR)

UAGUGCAGUCAC UGGCACAACG CGUUGCCCGG UAAGCCAAUC
GGGUAUACAC GGUCGUCAUACUGCAGACAG GGUUCUUCUA CUUUGCAAGA
UAGUCUAGAG UAGUAAAAUAAAUAGUAUAAG

SEQ ID NO:226 - HSP70-P2 (5′ UTR Enhancer)

GUCAGCUUUCAAACUCUUUGUUUCUUGUUUGUUGAUUGAGAAUA

SEQ ID NO:227 - HSP70-M1 (5′ UTR Enhancer)

CUCUCGCCUGAGAAAAAAAAUCCACGAACCAAUUUCUCAGCAACCAGCAG
CACG

SEQ ID NO:228 - HSP72-M2 (5′ UTR Enhancer)

ACCUGUGAGGGUUCGAAGGAAGUAGCAGUGUUUUUUGUUCCUAGAGGAAG
AG

SEQ ID NO:229 - HSP17.9 (5′ UTR Enhancer)

ACACAGAAACAUUCGCAAAAACAAAAUCCCAGUAUCAAAAUUCUUCUCUU
UUUUUCAUAUUUCGCAAAGAC

SEQ ID NO:230 - HSP70-P1 (5′ UTR Enhancer)

CAGAAAAAUUUGCUACAUUGUUUCACAAACUUCAAAUAUUAUUCAUUUAU
UU

SEQ ID NO:231 - Kozak Sequence

GCCACC

SEQ ID NO:232 - Kozak Sequence (Partial)

GCCA

SEQ ID NO:233 - SYNECHOCYSTIS sp. PCC6803 POTASSIUM CHANNEL (SynK) (5′ UTR)

AACUUAAAAAAAAAAAUCAAA

SEQ ID NO:234 - SYNTHETIC SEQUENCE (5′ UTR)

UCAAGCUUUUGGACCCUCGUACAGAAGCUAAUACGACUCACUAUAGGGAA
AUAAGAGAGAAAAGAAGAGUAAGAAGAAAUAUAAGAGCCACC

SEQ ID NO:235 - MOUSE BETA GLOBIN (5′ UTR)

CACAUUUGCUUCUGACAUAGUUGUGUUGACUCACAACCCCAGAAACAGAC
AUC

SEQ ID NO:236 - HUMAN BETA GLOBIN (5′ UTR)

ACAUUUGCUUCUGACACAACUGUGUUCACUAGCAACCUCAAACAGACACC

SEQ ID NO:237 - MOUSE ALBUMIN (5′ UTR)

UGCACACAGAUCACCUUUCCUAUCAACCCCACUAGCCUCUGGCAAA

SEQ ID NO:238 - HUMAN ALPHA GLOBIN (5′ UTR)

CAUAAACCCUGGCGCGCUCGCGGGCCGGCACUCUUCUGGUCCCCACAGAC
UCAGAGAGAACCCACC

SEQ ID NO:239 - HUMAN HAPTOGLOBIN (5′ UTR)

AUAAAAAGACCAGCAGAUGCCCCACAGCACUGCUCUUCCAGAGGCAAGAC
CAACCAAG

SEQ ID NO:240 - HUMAN TRANSTHYRETIN (5′ UTR)

AGACAAGGUUCAUAUUUGUAUGGGUUACUUAUUCUCUCUUUGUUGACUAA
GUCAAUAAUCAGAAUCAGCAGGUUUGCAGUCAGAUUGGCAGGGAUAAGCA
GCCUAGCUCAGGAGAAGUGAGUAUAAAAGCCCCAGGCUGGGAGCAGCCAU
CACAGAAGUCCACUCAUUCUUGGCAGG

SEQ ID NO:241 - HUMAN COMPLEMENT C3 (5′ UTR)

AGAUAAAAAGCCAGCUCCAGCAGGCGCUGCUCACUCCUCCCCAUCCUCUC
CCUCUGUCCCUCUGUCCCUCUGACCCUGCACUGUCCCAGCACC

SEQ ID NO:242 - HUMAN COMPLEMENT C5 (5′ UTR)

UAUAUCCGUGGUUUCCUGCUACCUCCAACC

SEQ ID NO:243 - HUMAN ALPHA-1-ANTITRYPSIN (5′ UTR)

GGCACCACCACUGACCUGGGACAGUGAAUCGACA

SEQ ID NO:244 - HUMAN ALPHA-1-ANTICHYMOTRYPSIN (5′ UTR)

AUUCAUGAAAAUCCACUACUCCAGACAGACGGCUUUGGAAUCCACCAGCU
ACAUCCAGCUCCCUGAGGCAGAGUUGAGA

SEQ ID NO:245 - HUMAN INTERLEUKIN 6 (5′ UTR)

AAUAUUAGAGUCUCAACCCCCAAUAAAUAUAGGACUGGAGAUGUCUGAGG
CUCAUUCUGCCCUCGAGCCCACCGGGAACGAAAGAGAAGCUCUAUCUCCC
CUCCAGGAGCCCAGCU

SEQ ID NO:246 - HUMAN FIBRINOGEN ALPHA CHAIN (5′ UTR)

AGGAUGGGAACUAGGAGUGGCAGCAAUCCUUUCUUUCAGCUGGAGUGCUC
CUCAGGAGCCAGCCCCACCCUUAGAAAAG

SEQ ID NO:247 - HUMAN APOLIPOPROTEIN E (5′ UTR)

AGGGGGAGCCCUAUAAUUGGACAAGUCUGGGAUCCUUGAGUCCUACUCAG
CCCCAGCGGAGGUGAAGGACGUCCUUCCCCAGGAGCCGACUGGCCAAUCA
CAGGCAGGAAG

SEQ ID NO:248 - ALANINE AMINOTRANSFERASE 1 (5′ UTR)

AGACGGGUGGGGCGGGGCCCAACUGUCCCCAGCUCCUUCAGCCCUUUCUG
UCCCUCCCAGUGAGGCCAGCUGCGGUGAAGAGGGUGCUCUCUUGCCUGGA
GUUCCCUCUGCUACGGCUGCCCCCUCCCAGCCCUGGCCCACUAAGCCAGA
CCCAGCUGUCGCCAUUCCCACUUCUGGUCCUGCCACCUCCUGAGCUGCCU
UCCCGCCUGGUCUGGGUAGAGUC

SEQ ID NO:249 - HHV (5′ UTR)

CAGAUCGCCUGGAGACGCCAUCCACGCUGUUUUGACCUCCAUAGAAGACA
CCGGGACCGAUCCAGCCUCCGCGGCCGGGAACGGUGCAUUGGAACGCGGA
UUCCCCGUGCCAAGAGUGACUCACCGUCCUUGACACG

SEQ ID NO:250 - ARC5-1 (5′ UTR)

GGGAGAAAGCUUACCAUGGUGCCCCAGGCCCUGCUCUUGGUCCCGCUGCU
GGUUUCCCCCUCUGCUUCGGCAAGUUCCCCAUCUACACCAUCCCCGACAA
GCUGGGGCCGUGGAGCCCCAUCGACAUCCACCACCUGUCCUGCCCCAACA
ACCUCGUGGUCGAGGACGAGGGCUGCACCAACCUGAGCGGGUUCUCCUAC

SEQ ID NO:251 - ARC5-2 (5′ UTR)

GGGGCGCUGCCUACGGAGGUGGCAGCCAUCUCCUUCUCGGCAUCAAGCUU
ACCAUGGUGCCCCAGGCCCUGCUCUUGGUCCCGCUGCUGGUGUUCCCCCU
CUGCUUCGGCAAGUUCCCCAUCUACACCAUCCCCGACAAGCUGGGGCCGU
GGAGCCCCAUCGACAUCCACCACCUGUCCUGCCCCAACAACCUCGUGGUC
GAGGACGAGGGCUGCACCAACCUGAGCGGGUUCUCCUAC

SEQ ID NO:252 - MOUSE GROWTH HORMONE (5′ UTR)

GAAUAAAUGUAUAGGGGGAAAGGCAGGAGCCUUGGGGUCGAGGAAAACAG
GUAGGGUAUAAAAAGGGCACGCAAGGGACCAAGUCCAGCAUCCUAGAGUC
CAGAUUCCAAACUGCUCAGAGUCCUGUGGACAGAUCACUGCUUGGCA

SEQ ID NO:253 - MOUSE HEMOGLOBIN ALPHA (5′ UTR)

GACACUUCUGAUUCUGACAGACUCAGGAAGAAACC

SEQ ID NO:254 - MOUSE HAPTOGLOBIN (5′ UTR)

UGCAAACACAGAAAUGGAGGAGGAGGGGAAGGAGGAGGAGGAGGAGAAGG
AGGAGGAGGUGGUGGUGGUGGUGGUGGGAUAAAACCCCUGAGGCAUAAAG
GGCUCGGCCGGAGUCAGCACAGCCCAGCCCUUCCAGAGAGAGGCAAGAGA
GGUCCACG

SEQ ID NO:255 - MOUSE TRANSTHYRETIN (5′ UTR)

CUAAUCUCCCUAGGCAAGGUUCAUAUUUGUGUAGGUUACUUAUUCUCCUU
UUGUUGACUAAGUCAAUAAUCAGAAUCAGCAGGUUUGGAGUCAGCUUGGC
AGGGAUCAGCAGCCUGGGUUGGAAGGAGGGGGUAUAAAAGCCCCUUCACC
AGGAGAAGCCGUCACACAGAUCCACAAGCUCCUGACAGG

SEQ ID NO:256 - MOUSE ANTITHROMBIN (5′ UTR)

AUAGGUAAUUUUAGAAAUAGAUCUGAUUUGUAUCUGAGACAUUUUAGUGA
AGUGGUGAGAUAUAAGACAUAAUCAGAAGACAUAUCUACCUGAAGACUUU
AAGGGGAGAGCUCCCUCCCCCACCUGGCCUCUGGACCUCUCAGAUUUAGG
GGAAAGAACCAGUUUUCGGAGUGAUCGUCUCAGUCAGCACCAUCUCUGUA
GGAGCAUCGGCC

SEQ ID NO:257 - MOUSE COMPLEMENT C3 (5′ UTR)

AGAGAGGAGAGCCAUAUAAAGAGCCAGCGGCUACAGCCCCAGCUCGCCUC
UGCCCACCCCUGCCCCUUACCCCUUCAUUCCUUCCACCUUUUUCCUUCAC
U

SEQ ID NO:258 - MOUSE COMPLEMENT C5 (5′ UTR)

UUUAAAAGGAAAGUGGUUACAGGGAGGCCAUGCCCAUGGGUUU

SEQ ID NO:259 - MOUSE HEPCIDIN (5′ UTR)

AGUCCUUAGACUGCACAGCAGAACAGAAGGCAUG

SEQ ID NO:260 - MOUSE ALPHA-1-ANTITRYPSIN (5′ UTR)

CCCCCAUAUCCCCCUUGGCUCCCAUUGCUUAAAUACAGACUAGGACAGGG
CUCUGUCUCCUCAGCCUCGGUCACCACCCAGCUCUGGGACAGCAAGCUGA
AA

SEQ ID NO:261 - MOUSE FIBRINOGEN ALPHA CHAIN (5′ UTR)

AGUCAGUCCUCCUUCGCUUCAGCUCCAGUUCUCCUCAUGAGCCAUCCCUA
AACGCAGACACC

SEQ ID NO:262 - APOLIPOPROTEIN E (5′ UTR)

UUUCCUCUGCCCUGCUGUGAAGGGGGAGAGAACAACCCGCCUCGUGACAG
GGGGCUGGCACAGCCCGCCCUAGCCCUGAGGAGGGGGCGGGACAGGGGGA
GUCCUAUAAUUGGACCGGUCUGGGAUCCGAUCCCCUGCUCAGACCCUGGA
GGCUAAGGACUUGUUUCGGAAGGAGCUGACUGGCCAAUCACAAUUGCGAA
G

SEQ ID NO:263 - ALANINE AMINOTRANSFERASE (5′ UTR)

GGCCGGCCACCGGGUUUGGGAGCAGCCCAGGCUCACCUUAACCGGAGCGG
UGCGGACGGUCCCGCGGCGACAGGGCUAAUCUCGGCAGGUUCGCG

SEQ ID NO:264 - CYTOCHROME P450, FAMILY 1(CYP1A2) (5′ UTR)

GUCCUGGACUGACUCCCACAACUCUGCCAGUCUCCAGCCCCUGCCCUUCA
GUGGUACAG

SEQ ID NO:265 - PLASMINOGEN (5′ UTR)

UUUAAGUCAACACCAGGAACUAGGACACAGUUGUCCAGGUGCUGUUGGCC
AGUCCCAAC

SEQ ID NO:266 - MOUSE MAJOR URINARY PROTEIN 3 (MUP3) (5′ UTR)

AAGGAGCUGGGGAGUGGAGUGUAGGCACUAUAACCUGAAAGACGUGGUCC
UGACAGGAGGACAAUUCUAUUCCCUACCAAA

SEQ ID NO:267 - MOUSE FVII (5′ UTR)

ACCAGCCAGAAGCCACAGUCUCAUC

SEQ ID NO:268 -ZHNF-1ALPHA (5′ UTR)

AAACAGAGCAGGCAGGGGCCCUGAUUCACUGGCCGCUGGGGCCAGGGUUG
GGGGCUGGGGGUGCCCACAGAGCUUGACUAGUGGGAUUUGGGGGGGCAGU
GGGUGCAGCGAGCCCGGUCCGUUGACUGCCAGCCUGCCGGCAGGUAGACA
CCGGCCGUGGGUGGGGGAGGCGGCUAGCUCAGUGGCCUUGGGCCGCGUGG
CCUGGUGGCAGCGGAGCC

SEQ ID NO:269 - MOUSE ALPHA-FETOPROTEIN (5′ UTR)

GGACUUCAGCAGGACUGCUCGAAACAUCCCACUUCCAGCACUGCCUGCGG
UGAAGGAACCAGCAGCC

SEQ ID NO:270 - MOUSE FIBRONECTIN (5′ UTR)

AGGGCCUCGUGGGGGGCGGGAAGGUACUGUCCCAUAUAAGCCUCUGCUCU
UGGGGCUCAACCGCUCGCACCCGCUGCGCUGCACAGGGGGAGAAAAGGAG
CCCAGGGUGUGAGCCGGACAACUUCUGGUCCUCUCCUUCCAUCUCCUUAC
CGGCGUCCCCACCUCAGGACUUUUCCCGCAGGCUGCGAGGGGACCCACAG
UUCGUGGCCACUUGCCUCCUGGGGAGGGCGACUCUCCUCCCAUCCACUCA
AG

SEQ ID NO:271 - MOUSE RETINOL BINDING PROTEIN 4, PLASMA (RBP4) (5′ UTR)

GGGGGAAAAAAAAACAGCCAAAAUAUGCCAAAAAGCUUCUCACAACAGCU
CCUCAGUAGAAGCAGGGGCCACUUGGGAAAGCCAGGGCCUGGACGCUAAU
GUUCCAGGCUACAUCAUAGGUCCCUUUUCGCUCAGUGAGGCCACCAUCAC
CACACCAUGGCCACGUAGGCCUCCAGCCAGGGCAACAGGACCUGGAGGCC
ACCCAAGACUGCAGCUGGCUGCCGCUGGGUCCCCGGGCCAGCUCUUGGCC
CCG

SEQ ID NO:272 - MOUSE PHOSPHOLIPID TRANSFER PROTEIN (PLTP) (5′ UTR)

GAACCGCGGCGAGGAGGGGGGUCGGAGGCCCAGACUUAUAAAGGCUGCUG
GACCCGCGCUACCCGCCAGACCCCGCCGCCCGGAUCCCCCGCGCUGCCUG
UCGCCCCACGUGACCACACUACUAAGCUUGGUCGCC

SEQ ID NO:273 - MOUSE ALANINE-GLYOXYLATE AMINOTRANSFERASE (AGXT) (5′ UTR)

AGGGACUCAUCAACCAGGCCUGGCCUCUGAGUUCAACGCAGAGCUAGCUG
GGAAAUGUUCCGGAUGUUGGCCAAGGCCAGUGUGACGCUGGGCUCCAGAG
CGGCAGGUUGGGUCCGGACC

SEQ ID NO:274 - ALDEHYDE DEHYDROGENASE 1 FAMILY, MEMBER L1 (ALDH1L1) (5′ UTR)

GCUGCCCCUGUGCUGACUGCUGACAGCUGACUGACGCUCGCAGCUAGCAG
GUACUUCUGGGUUGCUAGCCCAGAGCCCUGGGCCGGUGACCCUGUUUUCC
CUACUUCCCGUCUUUGACCUUGGGUGCCUUCCAACCUUCUGUUGCC

SEQ ID NO:275 - FUMARYLACETOACETATE HYDROLASE (FAH) (5′ UTR)

GGGUGCUAAAAGAAUCACUAGGGUGGGGAGGCGGUCCCAGUGGGGCGGGU
AGGGGUGUGUGCCAGGUGGUACCGGGUAUUGGCUGGAGGAAGGGCAGCCC
GGGGUUCGGGGCGGUCCCUGAAUCUAAAGGCCCUCGGCUAGUCUGAUCCU
UGCCCUAAGCAUAGUCCCGUUAGCCAACCCCCUACCCGCCGUGGGCUCUG
CUGCCCGGUGCUCGUCAGC

SEQ ID NO:276 - FRUCTOSE BISPHOSPHATASE 1 (FBP1) (5′ UTR)

AGGAGGACCUUGGCCAGCGGGCAGAAUGGCAGUUGGUAGAGGAAGGGAGC
AAGGGGGUGUUUCCUGGGACAGGGGGGCGGAGACCUGGAGACUAUAGGCU
CCCCCAGGACUCAAGUUCAUUGAGUUUCUGCAGACACUGAACGGCUUUCA
GUCUUCCCGCUGUGACUAUCACCUGUGGGCUCCACCUGCCUGCACCUUUA
GUCAGCACCUUUAGCCAGCACCUGCGCCAGACCCCAGCA

SEQ ID NO:277 - MOUSE GLYCINE N-METHYLTRANSFERASE (GNMT) (5′ UTR)

AGGCGCCGGUCAGG

SEQ ID NO:278 - MOUSE 4-HYDROXYPHENYLPYRUVIC ACID DIOXYGENASE (HPD) (5′ UTR)

ACCAUCAACC

SEQ ID NO:279 - HUMAN ANTITHROMBIN (5′ UTR)

UCUGCCCCACCCUGUCCUCUGGAACCUCUGCGAGAUUUAGAGGAAAGAAC
CAGUUUUCAGGCGGAUUGCCUCAGAUCACACUAUCUCCACUUGCCCAGCC
CUGUGGAAGAUUAGCGGCC

SEQ ID NO:280 - MOUSE BETA GLOBIN (3′ UTR)

ACCCCCUUUCCUGCUCUUGCCUGUGAACAAUGGUUAAUUGUUCCCAAGAG
AGCAUCUGUCAGUUGUUGGCAAAAUGAUAAAGACAUUUGAAAAUCUGUCU
UCUGACAAAUAAAAAGCAUUUAUUUCACUGCAAUGAUGUUUU

SEQ ID NO:281 - HUMAN BETA GLOBIN (3′ UTR)

GCUCGCUUUCUUGCUGUCCAAUUUCUAUUAAAGGUUCCUUUGUUCCCUAA
GUCCAACUACUAAACUGGGGGAUAUUAUGAAGGGCCUUGAGCAUCUGGAU
UCUGCCUAAUAAAAAACAUUUAUUUUCAUUGCAA

SEQ ID NO:282 - HUMAN GROWTH FACTOR (3′ UTR)

UGGCAUCCCUGUGACCCCUCCCCAGUGCCUCUCCUGGCCCUGGAAGUUGC
CACUCCAGUGCCCACCAGCCUUGUCCUAAUAAAAUUAAGUUGCAUCAUUU
UGUCUG

SEQ ID NO:283 - HUMAN ANTITHROMBIN (3′ UTR)

AAUGUUCUUAUUCUUUGCACCUCUUCCUAUUUUUGGUUUGUGAACAGAAG
UAAAAAUAAAUACAAACUACUUCCAUCUCA

SEQ ID NO:284 - HUMAN COMPLEMENT C3 (3′ UTR)

CCACACCCCCAUUCCCCCACUCCAGAUAAAGCUUCAGUUAUAUCUCACGU
GUCUGGAGUUCUUUGCCAAGAGGGAGAGGCUGAAAUCCCCAGCCGCCUCA
CCUGCAGCUCAGCUCCAUCCUACUUGAAACCUCACCUGUUCCCACCGCAU
UUUCUCCUGGCGUUCGCCUGCUAGUGUG

SEQ ID NO:285 - HUMAN HEPCIDIN (3′ UTR)

AACCUACCUGCCCUGCCCCCGUCCCCUCCCUUCCUUAUUUAUUCCUGCUG
CCCCAGAACAUAGGUCUUGGAAUAAAAUGGCUGGUUCUUUUGUUUUCCAA
A

SEQ ID NO:286 - HUMAN FIBRINOGEN ALPHA CHAIN (3′ UTR)

ACUAAGUUAAAUAUUUCUGCACAGUGUUCCCAUGGCCCCUUGCAUUUCCU
UCUUAACUCUCUGUUACACGUCAUUGAAACUACACUUUUUUGGUCUGUUU
UUGUGCUAGACUGUAAGUUCCUUGGGGGCAGGGCCUUUGUCUGUCUCAUC
UCUGUAUUCCCAAAUGCCUAACAGUACAGAGCCAUGACUCAAUAAAUACA
UGUUAAAUGGAUGAAUGAAUUCCUCUGAAACUCU

SEQ ID NO:287 - ALANINE AMINOTRANSFERASE 1 (3′ UTR)

GCACCCCAGCUGGGGCCAGGCUGGGUCGCCCUGGACUGUGUGCUCAGGAG
CCCUGGGAGGCUCUGGAGCCCACUGUACUUGCUCUUGAUGCCUGGCGGGG
UGGGGUGGGGGGGGUGCUGGGCCCCUGCCUCUCUGCAGGUCCCUAAUAAA
GCUGUGUGGCAGUCUGACUCC

SEQ ID NO:288 - MALAT (3′ UTR)

GAUUCGUCAGUAGGGUUGUAAAGGUUUUUCUUUUCCUGAGAAAACAACCU
UUUGUUUUCUCAGGUUUUGCUUUUUGGCCUUUCCCUAGCUUUAAAAAAAA
AAAAGCAAAA

SEQ ID NO:289 - ARC3-1 (3′ UTR)

GGACUAGUUAUAAGACUGACUAGCCCGAUGGGCCUCCCAACGGGCCCUCC
UCCCCUCCUUGCACCGAGAUUAAU

SEQ ID NO:290 - ARC3-2 (3′ UTR)

GGACUAGUGCAUCACAUUUAAAAGCAUCUCAGCCUACCAUGAGAAUAAGA
GAAAGAAAAUGAAGAUCAAUAGCUUAUUCAUCUCUUUUUCUUUUUCGUUG
GUGUAAAGCCAACACCCUGUCUAAAAAACAUAAAUUUCUUUAAUCAUUUU
GCCUCUUUUCUCUGUGCUUCAAUUAAUAAAAAAUGGAAAGAACCUAGAUC
U

SEQ ID NO:291 - MOUSE GROWTH HORMONE (3′ UTR)

CCACUCACCAGUGUCUCUGCUGCACUCUCCUGUGCCUCCCUGCCCCCUGG
CAACUGCCACCCCUGCGCUUUGUCCUAAUAAAAUUAAGAUGCAUCAUAUC
ACCCG

SEQ ID NO:292 - MOUSE HEMOGLOBIN ALPHA (3′ UTR)

GCUGCCUUCUGCGGGGCUUGCCUUCUGGCCAUGCCCUUCUUCUCUCCCUU
GCACCUGUACCUCUUGGUCUUUGAAUAAAGCCUGAGUAGGAAGAAAAAAA
AAAAA

SEQ ID NO:293 - MOUSE HAPTOGLOBIN (3′ UTR)

UUCAGGGCUCACUAGAAGGCUGCACAUGGCAGGGCAGGCUGGGAGCCAUG
GAAGAGGGGGAAGUGGAAGGGUUGGGCUAUACUCUGAUGGGUUCUAGCCC
UGCACUGCUCAGUCAACAAUAAAAAAAUGUGCUUUGGACCCAUAAAAAAA
AAAAAAAAAAAAA

SEQ ID NO:294 - MOUSE TRANSTHYRETIN (3′ UTR)

GAGACUCAGCCCAGGAGGACCAGGAUCUUGCCAAAGCAGUAGCAUCCCAU
UUGUACCAAAACAGUGUUCUUGCUCUAUAAACCGUGUUAGCAGCUCAGGA
AGAUGCCGUGAAGCAUUCUUAUUAAACCACCUGCUAUUUCAUUCAAACUG
UGUUUCUUUUUUAUUUCCUCAUUUUUCUCCCCUGCUCCUAAAACCCAAAA
UCUUCUAAAGAAUUCUAGAAGGUAUGCGAUCAAACUUUUUAAAGAAAGAA
AAUACUUUUUGACUCAUGGUUUAAAGGCAUCCUUUCCAUCUUGGGGAGGU
CAUGGGUGCUCCUGGCAACUUGCUUGAGGAAGAUAGGUCAGAAAGCAGAG
UGGACCAACCGUUCAAUGUUUUACAAGCAAAACAUACACUAAGCAUGGUC
UGUAGCUAUUAAAAGCACACAAUCUGAAGGGCUGUAGAUGCACAGUAGUG
UUUUCCCAGAGCAUGUUCAAAAGCCCUGGGUUCAAUCACAAUACUGAAAA
GUAGGCCAAAAAACAUUCUGAAAAUGAAAUAUUUGGGUUUUUUUUUAUAA
CCUUUAGUGACUAAAUAAAGACAAAUCUAAGAGACUAAAAAAAAAAAAAA
AAAA

SEQ ID NO:295 - MOUSE ANTITHROMBIN (3′ UTR)

AAUAUUCUUAAUCUUUGCACCUUUUCCUACUUUGGUGUUUGUGAAUAGAA
GUAAAAAUAAAUACGACUGCCACCUCACGAGAAUGGACUUUUCCACUUGA
AGACGAGAGACUGGAGUACAGAUGCUACACCACUUUUGGGCAAGUGAAGG
GGGAGCAGCCAGCCACGGUGGCACAAACCUAUAUCCUGGUGCUUUUGAAG
GUAGAAGCAGGGCGGUCAGGAGUUAAGGCCAGUUGAGGCUGGGCUGCAGA
GUGAAAGACCAUGUCUCAAGAUGGUCUUUCUCCUCCCCAAAGUAGAAAAG
AAAACCAUAAAAACAAGAGGUAAAUAUAUUACUAUUUCAUCUUAGAGGAU
AGCAGGCAUCUUGAAAGGGUAGAGGGACCUUAAAUUCUCAUUAUUGCCCC
CAUACUACAAACUAAAAAACAAACCCGAAUCAAUCUCCCAUAAAGACAGA
GAUUCAAAUAAGAGUAUUAAACGUUUUAUUUCUCAAACCACUCACAUGCA
UAAUGUUCUUAUACACAGUGUCAAAAUAAAGAGAAAUGCAUUUUUAUACA
AAAAAAAAAAA

SEQ ID NO:296 - MOUSE COMPLEMENT C3 (3′ UTR)

CUACAGCCCAGCCCUCUAAUAAAGCUUCAGUUGUAUUUCACCCAUC

SEQ ID NO:297 - MOUSE COMPLEMENT C5 (3′ UTR)

AAAGUUCUGCUGCACGAAGAUUCCUCCUGCGGCGGGGGGAUUGCUCCUCC
UCUGGCUUGGAAACCUAGCCUAGAAUCAGAUACACUUUCUUUAGAGUAAA
GCACAAGCUGAUGAGUUACGACUUUGUGAAAUGGAUAGCCUUGAGGGGAG
GCGAAAACAGGUCCCCCAAGGCUAUCAGAUGUCAGUGCCAAUAGACUGAA
ACAAGUCUGUAAAGUUAGCAGUCAGGGGUGUUGGUUGGGGCCGGAAGAAG
AGACCCACUGAAACUGUAGCCCCUUAUCAAAACAUAUCCUUGCUUGAAAG
AAAAAUACCAAGGACAGAAAAUGCCAUAAAAUCUUGACUUUGCACUC

SEQ ID NO:298 - MOUSE HEPCIDIN (3′ UTR)

CCUAGAGCCACAUCCUGACCUCUCUACACCCCUGCAGCCCCUCAACCCCA
UUAUUUAUUCCUGCCCUCCCCACCAAUGACCUUGAAAUAAAGACGAUUUU
AUUUUCAAAAAAAAAAAAAAAAAA

SEQ ID NO:299 - MOUSE ALPHA-1-ANTITRYPSIN (3′ UTR)

CCACCCUAAAAUGUCAUCCUUCCUUCUGAAUUGGGUUCCUUCCAUUAAAC
ACAGGCUGGCCUGGCUCGUGCCUGAUGCUACAGCAAGUCCUUGACUCUGU
GGGUUGUGUGUGUGUGUGUGUGUGUGUGUGUGUGUGUGUGUGUGUGUGUG
UGUGUGUGUGUGUGUGUGUCUGUGUGUGUGUGUGUCUUUAUGCCCUGAGU
UUGUUGUGGACUUGAGAUCAUAGUAUGUCUUGAUAUCUCCUCCAGCCAUG
CAAAUAGGUUGUGGGUAGAGGACUGUGGCUGAGACCACAGACUCUGGUCC
AAGAACCAUCUGCUCUAAAAAAAAUAAAUCUGUCAUCUCUGGAAAAUAAA
GAGGACAUGCUCAAUGACUCAGGGUCCAGC

SEQ ID NO:300 - MOUSE FIBRINOGEN ALPHA CHAIN (3′ UTR)

CUGAAGGGUUAGAAAGUGGGGGCUCUGUUUUCUUUGCUCGGUUAUCCGAG
AAGAAAGACAAAACGGAAGAUGAAGGUGUCACGGAUCUUGUGAACUUUUU
AAAACUUUCAAGGUGCUAUUCCAUUGUUCUUUGUACUGUAGCUAAAUGUA
ACUGAGAUGAGUUACUGCUUUGAAAAAAUAAAGUUUUACAUUUUUUCCAC
CCUUUAAAAAAAAAAAA

SEQ ID NO:301 - APOLIPOPROTEIN E (3′ UTR)

GUAUCCUUCUCCUGUCCUGCAACAACAUCCAUAUCCAGCCAGGUGGCCCU
GUCUCAAGCACCUCUCUGGCCCUCUGGUGGCCCUUGCUUAAUAAAGAUUC
UCCGAGCACAUUCUGAGUCUCUGUGAGUGAUUCAAAAAAAA

SEQ ID NO:302 - ALANINE AMINOTRANSFERASE (3′ UTR)

GGACGC CUCAGGCACC GGAGCCAGAC CCUCCCAAGA CCACCCAGGC
CUUCCUCAAG GACUCUGCCU CAGACCUCAG ACAGGCCACC AACGCUGUUC
AUCUUCAUUU CCCCAAGGAG ACUUCUUUCU UUGUGCCUUG AUGUUUGAGA
GUUCUUCGAG CAAACAGUGG UUUUGCAAUG UCUCACAGGC CCUGUUUUUG
UUUUUGUUUU UGUUUUGUUU UGUUUUGUUC UUUUUUUAAA UGCAACCAAA
GUAGAGUCAA CCUGCUCGGC AGAUGUACUU GGAUUCUCUG AAUCGCUAUU
CUGUUUGGAG AGUUCCUUUG GGUCUUAAGC AGCCAGAGUA CAUGGAAAUG
AGAUUAUGUC AGAUCUGGAG AAACAAGCAG GUGUUGGGAA AUAUGUGACU
UGACAUGAUA AGGGCUGGGA AUCCAGAAAU CAAUAGUGAG AUCCAUGAAA
UCAAACCCUG ACCAGUGUGA AAAUGUAGCC UUUUGGACAG UAAGCCUGCA
AGUCUAGUGA GAACUCAGAG AAAGCUGACC AUUCUGGUCU GAAGAUAGGC
AGCGCAUCAC AGGCAAGAAU AUCGAAGUCA GUAGUAGGAC AGGGGUCACA
UCAGAUACCA GCUCAAAUUG CACUAGCUAU CUAGAACAGU UUUCUCCAGG
UUUGCCUGAG CCUUGAUGCA UACCAUCGCC CUCUGCUGGU CGCAGCAGAG
AUAAGCAAGG GCUGAAAAUG GAGGCAAUCC UUUCCCAAGG CCCUGAAAGU
UGUUUUUCAU GGUUUCAAAC UGAAUUUGGC UCAUUUGUAA CUAACUGAUC
ACGGUGCCUG GUUACACUGG CUGCCAAGAA GGAGCGCAUG CAAUCUGAUU
CAGUGCUCUC UUCACAUCAG UUUCCUGCCU CCCUCCCUCA UCUGCGGACA
GCAUCCUAUC UCAUCAGGCU UCCCUGUGUG UCACAAAGUA GCAGCCACCA
AGCAAAUAUA UUCCUUGAAU UAGCACACCU GGGUGGGCCA UGUGCGCACC
AAGGAAACAG GUGCUAUAGG GAGCGCCAGG CCAGGCUUGU CUCUUAACUG
UCUCGUUCUU CAGUGAGAGU GGGAAAGCUG UCCGGAGCUC CCGCGCAGGA
GCCUGGGUAC CCACGCAGCG AGUCAAGGGA GUUUUCGGAG CCAGAGAGAG
AAAGAUGUGA AGGCUGUGGA GUAAGGCUGA AACCAGCCUC CUGCCCUAUA
GUCCCACACU GCAGGGGGUG CGACUUUAAA ACAGAACUUC AAGUUGUUAA
CACUCACAAG CAUUGCAUUA CUGUGAAGGA AGUAGCCGCA UCCAUAACAG
GAUGUGAUGG UCUACAGCUU UUCCUUUAAA AGCUGAAAAG GUACCAUGUG
UGCUCGCUAG GCAUAUAAUC CAGAUAUGCU CCAGAGUUCU GAGAUUCUUC
CAUGAAAGGU UAACUAGAAG CUAGAAUAUU UUUUUAUAUU UUUGUAACAA
UUGGCUUUUU UCAUGGGGGG AGGGGAGUAG AGGGUUAGUA UUUAUAGUCC
UAACAAGUCC AAAAAUUUUU AUAAGUGUCU UCAGAUUAUA AAUAACCCUC
CAAAUUUUGC AAUGUUUACA UGUUUUUUUU UUAAGAUGAC AAAUAUGCUU
GAUUUGCUUU UUAAAUAAAA GUUUAGCUGU UCUAAGAGAU UAACUUCAAG
UAGGAUGGCU GGUUAUGAUA GUUUGGAUUU UCUACAGGUU CUGUUGCCAU
GCCUUUUGGG UUUCAGCAUC ACUCGAGUCG CAGCAUGUGG GUGGGGCUGU
GGAAACCUGG CCAGGCUGGA CCUGGUCAGC CACACCUCAG AGACAUUGUU
UCCAUUUGGA UGUGAGCAGG CGCAGGCCUG CAUGCUCUUU CCUACUUAGC
AUCAUCAGUU CUUCCGCCUC CUUAGCAUGG UUCUUUGUAA CAGCCAUGCU
GGGAAGCUCU GAACAAUAAA AUACUUCCAG AGUGGU

SEQ ID NO:303 - CYTOCHROME P450, FAMILY 1(CYP1A2) (3′ UTR)

AGAUUGUCGAGGCAUCGGUGGGGCCGUCACCCUUGUUUCUUUUCCUUUUU
UAAAAAAAAAAAAAAAACAGCUUUUUUUUUUUUGAGAGAUACAAUUCUUU
CCCCAUUUAAUUCAUCUCCAAGCAAUUUUACAAUAGUGUCUAUCAUGUUC
ACCCCAUAACCCAUACUCAUUAGGACUUAUGAUUUAAGAUUCCUCCUACC
CUGUCUUGCUUGCCGCACCUCAUGCUAAUCUAGUUUUUGACUCAAUAGAU
UUGCCUACUCUGGCUGUCUCAUAUAAAUCGAAUGAAUUAUG

SEQ ID NO:304 - PLASMINOGEN (3′ UTR)

CUAGGUGGAAGGCCGAGCAAAACCUCUGCUUACUAAAGCUUACUGAAUAU
GGGGAGAGGGCUUAGGGUGUUUGGAAAAACUGACAGUAAUCAAACUGGGA
CACUACACUGAACCACAGCUUCCUGUCGCCCCUCAGCCCCUCCCCUUUUU
UUGUAUUAUUGUGGGUAAAAUUUUCCUGUCUGUGGACUUCUGGAUUUUGU
GACAAUAGACCAUCACUGCUGUGACCUUUGUUGAAAAUAAACUCGAUACU
UACUUUG

SEQ ID NO:305 - MOUSE MAJOR URINARY PROTEIN 3 (MUP3) (3′ UTR)

AGAAUGGCCUGAGC CUCCAGUGUU GAGUGGAGAC UUUUCACCAG
GACUCCAGCA UCAUCCCUUC CUAUCCAUAC AGACUCCCAU GCCAAGGUCU
GUGAUCUGCU CUCCACCUGU CUCACAGAGA AGUGCAAUCC CGUUCUCUCC
AGCAUGUUAC CUAGGAUAAC UCAUCAAGAA UCAAAGACUU UCUUUAAAUU
UCUCUUUGCC AACACAUGGA AAUUCUCCAU UGAUUUCUUU CCUGUCCUGU
UCAAUAAAUG AUUACACUUG CACUUAAAAA AAAAAAAA

SEQ ID NO:306 - MOUSE FVII (3′ UTR)

CUCCUUGGAUAGCC CAACCCGUCC CAAGAAGGAA GCUACGGCCU
GUGAAGCUGU UCUAUGGACU UUCCUGCUAU UCUUGUGUAA GGGAAGAGAA
UGAGAUAAAG AGAGAGUGAA GAAAGCAGAG GGGGAGGUAA AUGAGAGAGG
CUGGGAAAGG GGAAACAGAA AGCAGGGCCG GGGGAAGAGU CUAAGUUAGA
GACUCACAAA GAAACUCAAG AGGGGCUGGG CAGUGCAGUC ACAGUCAGGC
AGCUGAGGGG CAGGGUGUCC CUGAGGGAGG CGAGGCUCAG GCCUUGCUCC
CGUCUCCCCG UAGCUGCCUC CUGUCUGCAU GCAUUCGGUC UGCAGUACUA
CACAGUAGGU AUGCACAUGA GCACGUAGGA CACGUGAAUG UGCCGCAUGC
AUGUGCGUGC CUGUGUGUCC AUCAUUGGCA CUGUUGCUCA CUUGUGCUUC
CUGUGAGCAC CCUGUCUUGG UUUCAAUUAA AUGAGAAACA UGGUCAAAAA
AAAAAAAAAA AAAAA

SEQ ID NO:307 - HNF-1ALPHA (3′ UTR)

CCGUGGUGACUGCCU CCCAGGAGCU GGGUCCCCAG GGCCUGCACU
GCCUGCAUAG GGGGUGAGGA GGGCCGCAGC CACACUGCCU GGAGGAUAUC
UGAGCCUGCC AUGCCACCUG ACACAGGCUG CUGGCCUUCC CAGAAGUCUA
CGCAUUCAUU GACACUGCUG CUCCUCCAUC AUCAGGAAGG GAUGGCUCUG
AGGUGUCUCA GCCUGACAAG CGAGCCUCGA GGAGCUGGAG GACGGCCCAA
UCUGGGCAGU AUUGUGGACC ACCAUCCCUG CUGUUUAGAA UAGGAAAUUU
AAUGCUUGGG ACAGGAGUGG GGAAGCUCGU GGUGCCCGCA CCCCCCCAGU
CAGAGCCUGC AGGCCUUCAA GGAUCUGUGC UGAGCUCUGA GGCCCUAGAU
CAACACAGCU GCCUGCUGCC UCCUGCACCU CCCCAGGCCA UUCCACCCUG
CACCAGAGAC CCACGUGCCU GUUUGAGGAU UACCCUCCCC ACCACGGGGA
UUUCCUACCC AGCUGUUCUG CUAGGCUCGG GAGCUGAGGG GAAGCCACUC
GGGGCUCUCC UAGGCUUUCC CCUACCAAGC CAUCCCUUCU CCCAGCCCCA
GGACUGCACU UGCAGGCCAU CUGUUCCCUU GGAUGUGUCU UCUGAUGCCA
GCCUGGCAAC UUGCAUCCAC UAGAAAGGCC AUUUCAGGGC UCGGGUUGUC
AUCCCUGUUC CUUAGGACCU GCAACUCAUG CCAAGACCAC ACCAUGGACA
AUCCACUCCU CUGCCUGUAG GCCCCUGACA ACUUCCUUCC UGCUAUGAGG
GAGACCUGCA GAACUCAGAA GUCAAGGCCU GGGCAGUGUC UAGUGGAGAG
GGUACCAAGA CCAGCAGAGA GAAGCCACCU AAGUGGCCUG GGGGCUAGCA
GCCAUUCUGA GAAAUCCUGG GUCCCGAGCA GCCCAGGGAA ACACAGCACA
CAUGACUGUC UCCUCGGGCC UACUGCAGGG AACCUGGCCU UCAGCCAGCU
CCUUUGUCAU CCUGGACUGU AGCCUACGGC CAACCAUAAG UGAGCCUGUA
UGUUUAUUUA ACUUUUAGUA AAGUCAGUAA AAAGCAAAAA AAAAAAAAAA
AAA

SEQ ID NO:308 - MOUSE ALPHA-FETOPROTEIN (3′ UTR)

ACAUCUCCAGAAGGA AGAGUGGACA AAAAAAUGUG UUGACUCUUU
GGUGUGAGCC UUUUGGCUUA ACUGUAACUG CUAGUACUUU AACCACAUGG
UGAAGAUGUC CAUGUGAGAU UUCUAUACCU UAGGAAUAAA AACUUUUCAA
CUAUUUCUCU UCUCCUAGUC UGCUUUUUUU UUAUUAAAAA AUACUUUUUU
CCAUUU

SEQ ID NO:309 - MOUSE FIBRONECTIN (3′ UTR)

UCUUUCCAGCCCCA CCCUACAAGU GUCUCUCUAC CAAGGUCAAU
CCACACCCCA GUGAUGUUAG CAGACCCUCC AUCUUUGAGU GGUCCUUUCA
CCCUUAAGCC UUUUGCUCUG GAGCCAUGUU CUCAGCUUCA GCACAAUUUA
CAGCUUCUCC AAGCAUCGCC CCGUGGGAUG UUUUGAGACU UCUCUCCUCA
AUGGUGACAG UUGGUCACCC UGUUCUGCUU CAGGGUUUCA GUACUGCUCA
GUGUUGUUUA AGAGAAUCAA AAGUUCUUAU GGUUUGGUCU GGGAUCAAUA
GGGAAACACA GGUAGCCAAC UAGGAGGAAA UGUACUGAAU GCUAGUACCC
AAGACCUUGA GCAGGAAAGU CACCCAGACA CCUCUGCUUU CUUUUGCCAU
CUGACCUGCA GCACUGUCAG GACAUGGCCU GUGGCUGUGU GUUCAAACAC
CCCUCCCACA GGACUCACUU UGUCCCAACA AUUCAGAUUG CCUAGAAAUA
CCUUUCUCUU ACCUGUUUGU UAUUUAUCAA UUUUUCCCAG UAUUUUUAUA
CGGAAAAAAU UGUAUUGAAG ACACUUUGUA UGCAGUUGAU AAGAGGAAUU
CAGUAUAAUU AUGGUUGGUG AUUAUUUUUA UAAGCACAUG CCAACGCUUU
ACUACUGUGG AAAGACAAGU GUUUUAAUAA AAAGAUUUAC AUUCCAUGAU
GUGGACGUCA UUUCUUUUUU UUUUUAACAU CAUGUGUUUG GAGAG

SEQ ID NO:310 - MOUSE RETINOL BINDING PROTEIN 4, PLASMA (RBP4) (3′ UTR)

CAACGUCUAGGAUGUGAAG UUUGAAGAUU UCUGAUUAGC UUUCAUCCGG
UCUUCAUCUC UAUUUAUCUU AGAAGUUUAG UUUCCCCCAC CUCCCCUACC
UUCUCUAGGU GGACAUUAAA CCAUCGUCCA AAGUACAUGA GAGUCACUGA
CUCUGUUCAC ACAACUGUAU GUCUUACUGA AGGUCCCUGA AAGAUGUUUG
AGGCUUGGGA UUCCAAACUU GGUUUAUUAA ACAUAUAGUC ACCAUCUUCC
UAU

SEQ ID NO:311 - MOUSE PHOSPHOLIPID TRANSFER PROTEIN (PLTP) (3′ UTR)

GCCCAUCACCCC ACCUGGGUGG CUGGCAUUCA GGAACCUAAC UGAAGUCUUC
UCUGCACCCC CUGCCAACCC CUUCCCAUCU ACAGUGUUAG UGGUCCCGGU
GCCACAGAGA AGAGCCCAGU UGGAAGCUAU ACCCGAUUUA AUUCCAGAAU
UAGUCAACCA UCAAUUAGAA UCCAUCCACC CCCCUC

SEQ ID NO:312 - MOUSE ALANINE-GLYOXYLATE AMINOTRANSFERASE (AGXT) (3′ UTR)

GCAUCCUCUCA CCAGACUAUG CCCUCCUGGA GGGGCUGGGA AUAUAGCAAG
AACGAAAAGA CUGUGCAAGG CCUAGAGCCA GCAAAGAUGC UGAUGUAGCC
AGGCCAUGCC GGAAGGAGCA GGGUGAAGCU UCCCCUCUCC CUACAAAUGG
AACCUUGUGG AAACAGGAUG CUAAACACCU UCUGAUGGAG CUGUUGCCUG
CAGGCCACUG GUCUUUGGGA AUUUUCAAUA AAGUGCUUGC GAGGAAUCUC
CUA

SEQ ID NO:313 - ALDEHYDE DEHYDROGENASE 1 FAMILY, MEMBER L1 (ALDH1L1) (3′ UTR)

AGCCAAGACUGUGAU ACUUCUCCUG UACCCUGUUG ACCUCAGGGA
GUGCUGACCC UGUCUGGUGA CUUAGCACCC UCCUGUCCCC AGCACUGCUC
CUUUCAGCUG CUGGAGCUCU UGGCCUGGAC CCCUGCUGGU GACAGGACAC
CCUCUGAACA AUCAGAAGUG GCUCCAAGUG GAGUGAGCAG UCAUGUCCCC
CAUGAAUAAA AAUUGUGAGC AGAGGUCGCC UACAAAAAAA AAAAAAAA

SEQ ID NO:314 - FUMARYLACETOACETATE HYDROLASE (FAH) (3′ UTR)

AGCUCCGGAAG UCACAAGACA CACCCUUGCC UUAUGAGGAU CAUGCUACCA
CUGCAUCAGU CAGGAAUGAA UAAAGCUACU UUGAUUGUGG GAAAUGCCAC
AGAAAAAAAA AAAAAAA

SEQ ID NO:315 - FRUCTOSE BISPHOSPHATASE 1 (FBP1) (3′ UTR)

AGGCCAGCCUUGCC CCUGCCCCAG AGCAGAGCUC AAGUGACGCU
ACUCCAUUCU GCAUGUUGUA CAUUCCUAGA AACAAACCUA ACAGCGUGGA
UAGUUUCACA GCUUAAUGCU UUGCAAUGCC CAAGGUCACU UCAUCCUCAU
GCUAUAAUGC CACUGUAUCA GGUAAUAUAU AUUUUGAGUA GGUGAAGGAG
AAAUAAACAC AUCUUUCCUU UAUAAAUUA

SEQ ID NO:316 - MOUSE GLYCINE N-METHYLTRANSFERASE (GNMT) (3′ UTR)

GUUUCUCCGGCUCC CAGAAGCCCA UGCUCAGGCA AUGGCCCCUA
CCCUAAGACC AUCCCCUAAU GCAGAUAUUG CAUUUGGGUG CAGAUGUGGG
GGUCGGGCAA ACGGAGUAAA CAAUACAGUC UGCAUUCUCC AAAAAAAAAA
AA

SEQ ID NO:317 - MOUSE 4-HYDROXYPHENYLPYRUVIC ACID DIOXYGENASE (HPD) (3′ UTR)

GCCCCCAUCCACACAUGG ACCACGCAAA GUGCUGGACA CAUCAGUCAU
CUCCAACUGG CUGAAAGGCU GAACCUCAGG GCUCCACCCA CGUCAUGGCC
ACGCCCCCUC UAUUACAAGA GUCCGCCUUG CCUGAGUCCU CCCUGCUGAG
UAAAGCUACC CUCCCAGGUC CAAAAAAAAA AAAAAAAAAA AAAAAAAAAA
AAAAAAAAAA AAAAAAAA

SEQ ID NO:318 - LINKER

GGGS

SEQ ID NO:319 - LINKER

GPGP

SEQ ID NO:320 - LINKER

GGGSGGGS

| | |
|---|---|
| SEQ ID NO | Description¹ |
| SEQ ID NO:1 | mARM3325 (South Africa B.1.351; aka ARCT-165) |
| SEQ ID NO:2 | mARM3280 (D614G; aka ARCT-154) |
| SEQ ID NO:3 | mARM3333 (UK B.1.1.7) |
| SEQ ID NO:4 | mARM3346 (Brazil P. 1) |
| SEQ ID NO:5 | 5′ UTR (of SEQ ID NOs: 1 -4) |
| SEQ ID NO:6 | nsP1-nsP4 (of SEQ ID NOs: 1-4) |
| SEQ ID NO:7 | Intergenic region (of SEQ ID NOs: 1-4) |
| SEQ ID NO:8 | 3′ UTR (of SEQ ID NOs 1-4), with poly-A |
| SEQ ID NO:9 | 3′ UTR (of SEQ ID NOs: 1-4), without poly-A |
| SEQ ID NO: 10 | Transgene (nucleic acid sequence; mARM3325/SEQ ID NO: 1) |
| SEQ ID NO: 11 | Transgene (nucleic acid sequence; mARM3280/SEQ ID NO: 2) |
| SEQ ID NO: 12 | Transgene (nucleic acid sequence; mARM3333/SEQ ID NO: 3) |
| SEQ ID NO: 13 | Transgene (nucleic acid sequence; mARM3346/SEQ ID NO: 4) |
| SEQ ID NO: 14 | Transgene (amino acid sequence; mARM3325/SEQ ID NO: 1) |
| SEQ ID NO: 15 | Transgene (amino acid sequence; mARM3280/SEQ ID NO: 2) |
| SEQ ID NO: 16 | Transgene (amino acid sequence; mARM3333/SEQ ID NO: 3) |
| SEQ ID NO: 17 | Transgene (mARM3346/amino acid sequence; SEQ ID NO: 4) |
| SEQ ID NO: 18 | mARM3015 (Wuhan; aka ARCT-021) |
| SEQ ID NO: 19 | 5′ UTR (of mARM3015/SEQ ID NO:18) |
| SEQ ID NO:20 | nsP1-4 (of mARM3015/SEQ ID NO: 18) |
| SEQ ID NO:21 | Intergenic region (of SEQ ID NO: 18) |
| SEQ ID NO:22 | 3′ UTR (of mARM3015/SEQ NO: 18), with poly-A |
| SEQ ID NO:23 | 3′ UTR (of mARM3015/SEQ NO: 18), without poly-A |
| SEQ ID NO:24 | Transgene (nucleic acid sequence; mARM3015/SEQ ID NO: 18; codon-optimized) |
| SEQ ID NO:25 | Transgene (nucleic acid sequence; mARM3015/SEQ ID NO: 18; not codon-optimized) |
| SEQ ID NO:26 | Transgene (amino acid sequence; mARM3015/SEQ ID NO: 18) |
| SEQ ID NO:27 | Replicon sequence comprising SEQ ID NO: 19, SEQ ID NO:20, SEQ ID NO:21, and SEQ ID NO:22 (with poly-A) |
| SEQ ID NO:28 | Replicon sequence comprising SEQ ID NO: 19, SEQ ID NO:20, SEQ ID NO:21, and SEQ ID NO:23 (without poly-A) |
| SEQ ID NO:29 | mARM3326 (mRNA, South Africa B.1.351) |
| SEQ ID NO:30 | Transgene (nucleic acid sequence; mARM3326/SEQ ID NO:29) |
| SEQ ID NO:31 | Transgene (amino acid sequence; mARM3326/SEQ ID NO:29) |
| SEQ ID NO:32 | mARM3290 (mRNA, D614G; aka ARCT-143) |
| SEQ ID NO:33 | Transgene (nucleic acid sequence; mARM3290/SEQ ID NO: 32) |
| SEQ ID NO:34 | Transgene (amino acid sequence; mARM3290/SEQ ID NO: 32) |
| SEQ ID NO:35 | 5′ UTR (TEV) |
| SEQ ID NO:36 | 3′ UTR (Xbg), with poly-A |
| SEQ ID NO:37 | 3′ UTR (Xbg), without poly-A |
| SEQ ID NO:38 | 5′ UTR (alternative VEEV-derived sequence) |
| SEQ ID NO:39 | 5′ UTR (alternative VEEV-derived sequence) |
| SEQ ID NO:40 | mARM3124 (self-replicating RNA HA (A/Californial07/2009)) |
| SEQ ID NO:41 | 5′ UTR (of mARM3124/SEQ ID N0:40) |
| SEQ ID NO:42 | nsP1-nsP4 (of mARM3124/SEQ ID NO:40) |
| SEQ ID NO:43 | Intergenic region (of mARM3124/SEQ ID NO:40) |
| SEQ ID NO:44 | 3′ UTR (of mARM3124/SEQ ID NO: 40), with poly-A |
| SEQ ID NO:45 | 3′ UTR (of mARM3124/SEQ ID NO:40), without poly-A |
| SEQ ID NO:46 | Transgene (nucleic acid sequence; mARM3124/SEQ ID NO: 40) |
| SEQ ID NO:47 | Transgene (amino acid acid sequence; mARM3124/SEQ ID NO:40) |
| SEQ ID NO:48 | mARM3038 (mRNA HA (A/Californial07/2009)) |
| SEQ ID NO:49 | 5′ UTR (of mARM3038/SEQ ID NO:48) |
| SEQ ID NO:50 | 3′ UTR (of mARM3038/SEQ ID NO:48), with poly A |
| SEQ ID NO:51 | 3′ UTR (of mARM3038/SEQ ID NO:48), without poly A |
| SEQ ID NO:52 | Transgene (nucleic acid sequence; mARM3038/SEQ ID NO: 48) |
| SEQ ID NO:53 | Transgene (amino acid acid sequence; mARM3038/SEQ ID NO:48) |
| SEQ ID NO:54 | mmu-miR-451a (muscle) |
| SEQ ID NO:55 | mmu-miR-191-5p (muscle) |
| SEQ ID NO:56 | mmu-miR-181a-5p (muscle) |
| SEQ ID NO:57 | mmu-miR-99b-5p (muscle) |
| SEQ ID NO:58 | mmu-miR-10a-5p (muscle) |
| SEQ ID NO:59 | mmu-miR-10b-5p (muscle) |
| SEQ ID NO:60 | mmu-miR-193b-3p (muscle) |
| SEQ ID NO:61 | mmu-miR-22-3p (muscle) |
| SEQ ID NO:62 | mmu-miR-126a-5 (muscle)p |
| SEQ ID NO:63 | mmu-miR-92a-3p (muscle) |
| SEQ ID NO:64 | mmu-miR-125a-5p (muscle) |
| SEQ ID NO:65 | mmu-miR-378a-3p (muscle) |
| SEQ ID NO:66 | mmu-miR-143-3p (muscle) |
| SEQ ID NO:67 | mmu-let-7a-5p (muscle) |
| SEQ ID NO:68 | mmu-let-7b-5p (muscle) |
| SEQ ID NO:69 | mmu-let-7c-5p (muscle) |
| SEQ ID NO:70 | mmu-let-7f-5p (muscle) |
| SEQ ID NO:71 | mmu-miR-126b-3p (muscle) |
| SEQ ID NO:72 | mmu-miR-423-3p (muscle) |
| SEQ ID NO:73 | mmu-miR-30a-5p (muscle) |
| SEQ ID NO:74 | mmu-miR-30d-5p (muscle) |
| SEQ ID NO:75 | mmu-miR-30e-5p (muscle) |
| SEQ ID NO:76 | mmu-miR-26a-5p (muscle) |
| SEQ ID NO:77 | mmu-miR-27b-3p (muscle) |
| SEQ ID NO:78 | mmu-miR-133a-3p.1 (muscle) |
| SEQ ID NO:79 | mmu-miR-133a-3p.2 (muscle) |
| SEQ ID NO:80 | hsa-miR-486-5p (muscle) |
| SEQ ID NO:81 | hsa-miR-486-3p (muscle) |
| SEQ ID NO:82 | hsa-miR-451a (muscle) |
| SEQ ID NO:83 | hsa-miR-423-3p (muscle) |
| SEQ ID NO:84 | hsa-miR-378a-3p (muscle) |
| SEQ ID NO:85 | hsa-miR-193b-3p (muscle) |
| SEQ ID NO:86 | hsa-miR-191-5p (muscle) |
| SEQ ID NO:87 | hsa-miR-181a-5p (muscle) |
| SEQ ID NO:88 | hsa-miR-143-3p (muscle) |
| SEQ ID NO:89 | hsa-miR-133a-3p.2 (muscle) |
| SEQ ID NO:90 | hsa-miR-133a-3p.1 (muscle) |
| SEQ ID NO:91 | hsa-miR-125a-5p (muscle) |
| SEQ ID NO:92 | hsa-miR-101-3p.2 (muscle) |
| SEQ ID NO:93 | hsa-miR-101-3p.1 (muscle) |
| SEQ ID NO:94 | hsa-miR-99b-5p (muscle) |
| SEQ ID NO:95 | hsa-miR-30a-5p (muscle) |
| SEQ ID NO:96 | hsa-miR-30d-5p (muscle) |
| SEQ ID NO:97 | hsa-miR-30e-5p (muscle) |
| SEQ ID NO:98 | hsa-miR-27b-3p (muscle) |
| SEQ ID NO:99 | hsa-miR-26a-5p (muscle) |
| SEQ ID NO:100 | hsa-miR-92a-3p (muscle) |
| SEQ ID NO:101 | hsa-miR-22-3p (muscle) |
| SEQ ID NO:102 | hsa-miR-10a-5p (muscle) |
| SEQ ID NO:103 | hsa-miR-10b-5p (muscle) |
| SEQ ID NO:104 | hsa-let-7a-5p (muscle) |
| SEQ ID NO:105 | hsa-let-7b-5p (muscle) |
| SEQ ID NO:106 | hsa-let-7c-5p (muscle) |
| SEQ ID NO:107 | hsa-let-7f-5p (muscle) |
| SEQ ID NO:108 | mmu-miR-191-5p (dendritic cells) |
| SEQ ID NO:109 | mmu-miR-181a-5p (dendritic cells) |
| SEQ ID NO:110 | mmu-miR-181b-5p (dendritic cells) |
| SEQ ID NO:111 | mmu-miR-99b-5p (dendritic cells) |
| SEQ ID NO:112 | mmu-miR-10a-5p (dendritic cells) |
| SEQ ID NO:113 | mmu-miR-29a-3p (dendritic cells) |
| SEQ ID NO:114 | mmu-miR-16-5p (dendritic cells) |
| SEQ ID NO:115 | mmu-miR-22-3p (dendritic cells) |
| SEQ ID NO:116 | mmu-miR-21a-5p (dendritic cells) |
| SEQ ID NO:117 | mmu-miR-142a-5p (dendritic cells) |
| SEQ ID NO:118 | mmu-miR-25-3p (dendritic cells) |
| SEQ ID NO:119 | mmu-miR-92a-3p (dendritic cells) |
| SEQ ID NO:120 | mmu-miR-148a-3p (dendritic cells) |
| SEQ ID NO:121 | mmu-miR-378a-3p (dendritic cells) |
| SEQ ID NO:122 | mmu-miR-146b-5p (dendritic cells) |
| SEQ ID NO:123 | mmu-miR-27b-5p (dendritic cells) |
| SEQ ID NO:124 | mmu-let-7a-5p (dendritic cells) |
| SEQ ID NO:125 | mmu-let-7f-5p (dendritic cells) |
| SEQ ID NO:126 | mmu-let-7g-5p (dendritic cells) |
| SEQ ID NO:127 | mmu-let-7i-5p (dendritic cells) |
| SEQ ID NO:128 | mmu-miR-103-3p (dendritic cells) |
| SEQ ID NO:129 | mmu-miR-221-3p (dendritic cells) |
| SEQ ID NO:130 | mmu-miR-222-3p (dendritic cells) |
| SEQ ID NO:131 | mmu-miR-24-3p (dendritic cells) |
| SEQ ID NO:132 | mmu-miR-27a-5p (dendritic cells) |
| SEQ ID NO:133 | mmu-miR-30d-5p (dendritic cells) |
| SEQ ID NO:134 | mmu-miR-223-3p (dendritic cells) |
| SEQ ID NO:135 | mmu-miR-223-5p (dendritic cells) |
| SEQ ID NO:136 | mmu-miR-155-5p (dendritic cells) |
| SEQ ID NO:137 | mmu-miR-26a-5p(dendritic cells) |
| SEQ ID NO:138 | mmu-miR-26b-5p (dendritic cells) |
| SEQ ID NO:139 | mmu-miR-27a-3p (dendritic cells) |
| SEQ ID NO:140 | mmu-miR-27b-3p (dendritic cells) |
| SEQ ID NO:141 | hsa-miR-423-5p (dendritic cells) |
| SEQ ID NO:142 | hsa-miR-423-3p (dendritic cells) |
| SEQ ID NO:143 | hsa-miR-378a-3p (dendritic cells) |
| SEQ ID NO:144 | hsa-miR-342-3p (dendritic cells) |
| SEQ ID NO:145 | hsa-miR-223-5p (dendritic cells) |
| SEQ ID NO:146 | hsa-miR-223-3p (dendritic cells) |
| SEQ ID NO:147 | hsa-miR-191-5p (dendritic cells) |
| SEQ ID NO:148 | hsa-miR-186-5p (dendritic cells) |
| SEQ ID NO:149 | hsa-miR-181a-5p (dendritic cells) |
| SEQ ID NO:150 | hsa-miR-146b-5p (dendritic cells) |
| SEQ ID NO:151 | hsa-miR-142-5p (dendritic cells) |
| SEQ ID NO:152 | hsa-miR-142-3p.2 (dendritic cells) |
| SEQ ID NO:153 | hsa-miR-142-3p.1 (dendritic cells) |
| SEQ ID NO:154 | hsa-miR-140-3p.2 (dendritic cells) |
| SEQ ID NO:155 | hsa-miR-140-3p.1 (dendritic cells) |
| SEQ ID NO:156 | hsa-miR-103a-3p (dendritic cells) |
| SEQ ID NO:157 | hsa-miR-107 (dendritic cells) |
| SEQ ID NO:158 | hsa-miR-30a-5p (dendritic cells) |
| SEQ ID NO:159 | hsa-miR-30c-5p (dendritic cells) |
| SEQ ID NO:160 | hsa-miR-30d-5p (dendritic cells) |
| SEQ ID NO:161 | hsa-miR-30e-5p (dendritic cells) |
| SEQ ID NO:162 | hsa-miR-28-3p (dendritic cells) |
| SEQ ID NO:163 | hsa-miR-27b-5p (dendritic cells) |
| SEQ ID NO:164 | hsa-miR-27a-5p (dendritic cells) |
| SEQ ID NO:165 | hsa-miR-27a-3p (dendritic cells) |
| SEQ ID NO:166 | hsa-miR-27b-3p (dendritic cells) |
| SEQ ID NO:167 | hsa-miR-26a-5p (dendritic cells) |
| SEQ ID NO: 168 | hsa-miR-26b-5p (dendritic cells) |
| SEQ ID NO:169 | hsa-miR-25-3p (dendritic cells) |
| SEQ ID NO:170 | hsa-miR-92a-3p (dendritic cells) |
| SEQ ID NO:171 | hsa-miR-24-3p(dendritic cells) |
| SEQ ID NO:172 | hsa-miR-22-3p (dendritic cells) |
| SEQ ID NO:173 | hsa-miR-21-5p (dendritic cells) |
| SEQ ID NO:174 | hsa-miR-21-3p (dendritic cells) |
| SEQ ID NO:175 | hsa-miR-16-5p (dendritic cells) |
| SEQ ID NO:176 | hsa-let-7a-5p (dendritic cells) |
| SEQ ID NO:177 | hsa-let-7b-5p (dendritic cells) |
| SEQ ID NO:178 | hsa-let-7c-5p (dendritic cells) |
| SEQ ID NO:179 | hsa-let-7d-5p (dendritic cells) |
| SEQ ID NO:180 | hsa-let-7e-5p (dendritic cells) |
| SEQ ID NO:181 | hsa-let-7f-5p (dendritic cells) |
| SEQ ID NO:182 | hsa-let-7g-5p (dendritic cells) |
| SEQ ID NO:183 | hsa-let-7i-5p (dendritic cells) |
| SEQ ID NO:184 | hsa-miR-98-5p (dendritic cells) |
| SEQ ID NO:185 | Codon optimized region of nsP1-4 (nucleotide 463 to nucleotide 7455) |
| SEQ ID NO:186 | Self-replicating RNA with codon-optimized nsP1-4 and Luciferase Transgene |
| SEQ ID NO:187 | nsP1-4 amino acid sequence (encoded by SEQ ID NO: 6 and SEQ ID NO: 42) |
| SEQ ID NO:188 | nsP1-4 amino acid sequence (encoded by SEQ ID NO: 20) |
| SEQ ID NO:189 | TEV (5′ UTR) |
| SEQ ID NO:190 | AT1G58420 (5′ UTR) |
| SEQ ID NO:191 | ARC5-2 (5′ UTR) |
| SEQ ID NO:192 | HCV (5′ UTR) |
| SEQ ID NO:193 | HUMAN ALBUMIN (5′ UTR) |
| SEQ ID NO:194 | EMCV (5′ UTR) |
| SEQ ID NO:195 | AT1G67090 (5′ UTR) |
| SEQ ID NO:196 | AT1G35720 (5′ UTR) |
| SEQ ID NO:197 | AT5G45900 (5′ UTR) |
| SEQ ID NO:198 | AT5G61250 (5′ UTR) |
| SEQ ID NO: 199 | AT5G46430 (5′ UTR) |
| SEQ ID NO:200 | AT5 G47110 (5′ UTR) |
| SEQ ID NO:201 | AT1G03110 (5′ UTR) |
| SEQ ID NO:202 | AT3G12380 (5′ UTR) |
| SEQ ID NO:203 | AT5G45910 (5′ UTR) |
| SEQ ID NO:204 | AT1G07260 (5′ UTR) |
| SEQ ID NO:205 | AT3G55500 (5′ UTR) |
| SEQ ID NO:206 | AT3G46230 (5′ UTR) |
| SEQ ID NO:207 | AT2G36170 (5′ UTR) |
| SEQ ID NO:208 | AT1G10660 (5′ UTR) |
| SEQ ID NO:209 | AT4G14340 (5′ UTR) |
| SEQ ID NO:210 | AT1G49310 (5′ UTR) |
| SEQ ID NO:211 | AT4G14360 (5′ UTR) |
| SEQ ID NO:212 | AT1G28520 (5′ UTR) |
| SEQ ID NO:213 | AT1G20160 (5′ UTR) |
| SEQ ID NO:214 | AT5G37370 (5′ UTR) |
| SEQ ID NO:215 | AT4G11320 (5′ UTR) |
| SEQ ID NO:216 | AT5G40850 (5′ UTR) |
| SEQ ID NO:217 | AT1G06150 (5′ UTR) |
| SEQ ID NO:218 | AT2G26080 (5′ UTR) |
| SEQ ID NO:219 | XBG (3′UTR) |
| SEQ ID NO:220 | HUMAN HAPTOGLOBIN (3′UTR) |
| SEQ ID NO:221 | HUMAN APOLIPOPROTEIN E (3′UTR) |
| SEQ ID NO:222 | HCV (3′UTR) |
| SEQ ID NO:223 | MOUSE ALBUMIN (3′UTR) |
| SEQ ID NO:224 | HUMAN ALPHA GLOBIN (3′UTR) |
| SEQ ID NO:225 | EMCV (3′UTR) |
| SEQ ID NO:226 | HSP70-P2 (5′ UTR Enhancer) |
| SEQ ID NO:227 | HSP70-M1 (5′ UTR Enhancer) |
| SEQ ID NO:228 | HSP72-M2 (5′ UTR Enhancer) |
| SEQ ID NO:229 | HSP17.9 (5′ UTR Enhancer) |
| SEQ ID NO:230 | HSP70-P1 (5′ UTR Enhancer) |
| SEQ ID NO:231 | Kozak Sequence |
| SEQ ID NO:232 | Kozak Sequence (Partial) |
| SEQ ID NO:233 | SYNECHOCYSTIS sp. PCC6803 POTASSIUM CHANNEL (SynK) (5′ UTR) |
| SEQ ID NO:234 | SYNTHETIC SEQUENCE (5′ UTR) |
| SEQ ID NO:235 | MOUSE BETA GLOBIN (5′ UTR) |
| SEQ ID NO:236 | HUMAN BETA GLOBIN (5′ UTR) |
| SEQ ID NO:237 | MOUSE ALBUMIN (5′ UTR) |
| SEQ ID NO:238 | HUMAN ALPHA GLOBIN (5′ UTR) |
| SEQ ID NO:239 | HUMAN HAPTOGLOBIN (5′ UTR) |
| SEQ ID NO:240 | HUMAN TRANSTHYRETIN (5′ UTR) |
| SEQ ID NO:241 | HUMAN COMPLEMENT C3 (5′ UTR) |
| SEQ ID NO:242 | HUMAN COMPLEMENT C5 (5′ UTR) |
| SEQ ID NO:243 | HUMAN ALPHA-1-ANTITRYPSIN (5′ UTR) |
| SEQ ID NO:244 | HUMAN ALPHA-1-ANTICHYMOTRYPSIN (5′ UTR) |
| SEQ ID NO:245 | HUMAN INTERLEUKIN 6 (5′ UTR) |
| SEQ ID NO:246 | HUMAN FIBRINOGEN ALPHA CHAIN (5′ UTR) |
| SEQ ID NO:247 | HUMAN APOLIPOPROTEIN E (5′ UTR) |
| SEQ ID NO:248 | ALANINE AMINOTRANSFERASE 1 (5′ UTR) |
| SEQ ID NO:249 | HHV (5′ UTR) |
| SEQ ID NO:250 | ARC5-1 (5′ UTR) |
| SEQ ID NO:251 | ARC5-2 (5′ UTR) |
| SEQ ID NO:252 | MOUSE GROWTH HORMONE (5′ UTR) |
| SEQ ID NO:253 | MOUSE HEMOGLOBIN ALPHA (5′ UTR) |
| SEQ ID NO:254 | MOUSE HAPTOGLOBIN (5′ UTR) |
| SEQ ID NO:255 | MOUSE TRANSTHYRETIN (5′ UTR) |
| SEQ ID NO:256 | MOUSE ANTITHROMBIN (5′ UTR) |
| SEQ ID NO:257 | MOUSE COMPLEMENT C3 (5′ UTR) |
| SEQ ID NO:258 | MOUSE COMPLEMENT C5 (5′ UTR) |
| SEQ ID NO:259 | MOUSE HEPCIDIN (5′ UTR) |
| SEQ ID NO:260 | MOUSE ALPHA-1-ANTITRYPSIN (5′ UTR) |
| SEQ ID NO:261 | MOUSE FIBRINOGEN ALPHA CHAIN (5′ UTR) |
| SEQ ID NO:262 | APOLIPOPROTEIN E (5′ UTR) |
| SEQ ID NO:263 | ALANINE AMINOTRANSFERASE (5′ UTR) |
| SEQ ID NO:264 | CYTOCHROME P450, FAMILY 1(CYP1A2) (5′ UTR) |
| SEQ ID NO:265 | PLASMINOGEN (5′ UTR) |
| SEQ ID NO:266 | MOUSE MAJOR URINARY PROTEIN 3 (MUP3) (5′ UTR) |
| SEQ ID NO:267 | MOUSE FVII (5′ UTR) |
| SEQ ID NO:268 | HNF-1 ALPHA (5′ UTR) |
| SEQ ID NO:269 | MOUSE ALPHA-FETOPROTEIN (5′ UTR) |
| SEQ ID NO:270 | MOUSE FIBRONECTIN (5′ UTR) |
| SEQ ID NO:271 | MOUSE RETINOL BINDING PROTEIN 4, PLASMA (RBP4) (5′ UTR) |
| SEQ ID NO:272 | MOUSE PHOSPHOLIPID TRANSFER PROTEIN (PLTP) (5′ UTR) |
| SEQ ID NO:273 | MOUSE ALANINE-GLYOXYLATE AMINOTRANSFERASE (AGXT) (5′ UTR) |
| SEQ ID NO:274 | ALDEHYDE DEHYDROGENASE 1 FAMILY, MEMBER L1 (ALDH1L1) (5′ UTR) |
| SEQ ID NO:275 | FUMARYLACETOACETATE HYDROLASE (FAH) (5′ UTR) |
| SEQ ID NO:276 | FRUCTOSE BISPHOSPHATASE 1 (FBP1) (5′ UTR) |
| SEQ ID NO:277 | MOUSE GLYCINE N-METHYLTRANSFERASE (GNMT) (5′ UTR) |
| SEQ ID NO:278 | MOUSE 4-HYDROXYPHENYLPYRUVIC ACID DIOXYGENASE (HPD) (5′ UTR) |
| SEQ ID NO:279 | HUMAN ANTITHROMBIN (5′ UTR) |
| SEQ ID NO:280 | MOUSE BETA GLOBIN (3′ UTR) |
| SEQ ID NO:281 | HUMAN BETA GLOBIN (3′ UTR) |
| SEQ ID NO:282 | HUMAN GROWTH FACTOR (3′ UTR) |
| SEQ ID NO:283 | HUMAN ANTITHROMBIN (3′ UTR) |
| SEQ ID NO:284 | HUMAN COMPLEMENT C3 (3′ UTR) |
| SEQ ID NO:285 | HUMAN HEPCIDIN (3′ UTR) |
| SEQ ID NO:286 | HUMAN FIBRINOGEN ALPHA CHAIN (3′ UTR) |
| SEQ ID NO:287 | ALANINE AMINOTRANSFERASE 1 (3′ UTR) |
| SEQ ID NO:288 | MALAT (3′ UTR) |
| SEQ ID NO:289 | ARC3-1 (3′ UTR) |
| SEQ ID NO:290 | ARC3-2 (3′ UTR) |
| SEQ ID NO:291 | MOUSE GROWTH HORMONE (3′ UTR) |
| SEQ ID NO:292 | MOUSE HEMOGLOBIN ALPHA (3′ UTR) |
| SEQ ID NO:293 | MOUSE HAPTOGLOBIN (3′ UTR) |
| SEQ ID NO:294 | MOUSE TRANSTHYRETIN (3′ UTR) |
| SEQ ID NO:295 | MOUSE ANTITHROMBIN (3′ UTR) |
| SEQ ID NO:296 | MOUSE COMPLEMENT C3 (3′ UTR) |
| SEQ ID NO:297 | MOUSE COMPLEMENT C5 (3′ UTR) |
| SEQ ID NO:298 | MOUSE HEPCIDIN (3′ UTR) |
| SEQ ID NO:299 | MOUSE ALPHA-1-ANTITRYPSIN (3′ UTR) |
| SEQ ID NO:300 | MOUSE FIBRINOGEN ALPHA CHAIN (3′ UTR) |
| SEQ ID NO:301 | APOLIPOPROTEIN E (3′ UTR) |
| SEQ ID NO:302 | ALANINE AMINOTRANSFERASE (3′ UTR) |
| SEQ ID NO:303 | CYTOCHROME P450, FAMILY 1(CYP1A2) (3′ UTR) |
| SEQ ID NO:304 | PLASMINOGEN (3′ UTR) |
| SEQ ID NO:305 | MOUSE MAJOR URINARY PROTEIN 3 (MUP3) (3′ UTR) |
| SEQ ID NO:306 | MOUSE FVII (3′ UTR) |
| SEQ ID NO:307 | HNF-1ALPHA (3′ UTR) |
| SEQ ID NO:308 | MOUSE ALPHA-FETOPROTEIN (3′ UTR) |
| SEQ ID NO:309 | MOUSE FIBRONECTIN (3′ UTR) |
| SEQ ID NO:310 | MOUSE RETINOL BINDING PROTEIN 4, PLASMA (RBP4) (3′ UTR) |
| SEQ ID NO:311 | MOUSE PHOSPHOLIPID TRANSFER PROTEIN (PLTP) (3′ UTR) |
| SEQ ID NO:312 | MOUSE ALANINE-GLYOXYLATE AMINOTRANSFERASE (AGXT) (3′ UTR) |
| SEQ ID NO:313 | ALDEHYDE DEHYDROGENASE 1 FAMILY, MEMBER L1 (ALDH1L1) (3′ UTR) |
| SEQ ID NO:314 | FUMARYLACETOACETATE HYDROLASE (FAH) (3′ UTR) |
| SEQ ID NO:315 | FRUCTOSE BISPHOSPHATASE 1 (FBP1) (3′ UTR) |
| SEQ ID NO:316 | MOUSE GLYCINE N-METHYLTRANSFERASE (GNMT) (3′ UTR) |
| SEQ ID NO:317 | MOUSE 4-HYDROXYPHENYLPYRUVIC ACID DIOXYGENASE (HPD) (3′ UTR) |
| SEQ ID NO:318 | Linker (Amino acid) |
| SEQ ID NO:319 | Linker (Amino acid) |
| SEQ ID NO:320 | Linker (Amino acid) |

¹hsa - homo sapiens; mmu - mus musulus; descriptions of constructs sequences occur in are provided as non-limiting examples

Unless defined otherwise, all technical and scientific terms used herein have the same meaning as is commonly understood by one of skill in the art to which this invention belongs.

Any and all references and citations to other documents, such as patents, patent applications, patent publications, journals, books, papers, web contents, that have been made throughout this disclosure are hereby incorporated herein in their entirety for all purposes.

Although the invention has been described with reference to the above examples, it will be understood that modifications and variations are encompassed within the spirit and scope of the invention. Accordingly, the invention is limited only by the following claims.

1. An RNA molecule comprising:
(a) a first polynucleotide encoding one or more viral replication proteins, wherein one or more miRNA binding sites in the first polynucleotide have been modified as compared to a reference polynucleotide; and
(b) a second polynucleotide comprising a first transgene encoding a first antigenic protein or a fragment thereof.

2. The RNA molecule of claim 1, wherein modification of the one or more miRNA binding sites reduces or eliminates miRNA binding.

3. The RNA molecule of claim 1, wherein two, three, four, five, six, seven, eight, nine, ten, 11, 12, 13, 14, or 15 miRNA binding sites in the first polynucleotide have been modified.

4. The RNA molecule of claim 1, wherein the one or more miRNA binding sites are selected from regions that bind a miRNA having a sequence of SEQ ID NOs:58, 59, 72, 80, 81, 83, 101, 102, 103, 112, 113, 114, 128, 131, 142, 156, 157, 171, 175, and any combination thereof.

5. The RNA molecule of claim 1, wherein the one or more viral replication proteins are alphavirus proteins.

6. (canceled)

7. The RNA molecule of claim 1, wherein the first polynucleotide encodes a polyprotein comprising an alphavirus nsP1 protein, an alphavirus nsP2 protein, an alphavirus nsP3 protein, and an alphavirus nsP4 protein .

8. (canceled)

9. The RNA molecule of claim 1, wherein the first polynucleotide comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to a sequence of SEQ ID NO:6.

10. (canceled)

11. The RNA molecule of claim 1, wherein the first polynucleotide encodes a polyprotein comprising a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to a sequence of SEQ ID NO:187.

12. The RNA molecule of claim 1, further comprising
(i) a 5′ untranslated region (UTR); or
(ii) a 5′ UTR comprising an alphavirus 5′ UTR sequence; or
(iii) a 5′ UTR comprising a sequence of SEQ ID NO:5; or
(iv) a 3′ UTR; or
(v) a 3′ UTR comprising an alphavirus 3′ UTR sequence; or
(vi) a 3′ UTR comprising a sequence of SEQ ID NO:9; or
(vii) a 3′ UTR comprising a poly-A sequence; or
(viii) any combination thereof.

13. (canceled)

14. (canceled)

15. (canceled)

16. (canceled)

17. (canceled)

18. (canceled)

19. (canceled)

20. (canceled)

21. (canceled)

22. (canceled)

23. The RNA molecule of claim 1, wherein the first antigenic protein is a viral protein, a bacterial protein, a fungal protein, a protozoan protein, or a parasite protein.

24. The RNA molecule of claim 23, wherein the viral protein is a coronavirus protein, an orthomyxovirus protein, a paramyxovirus protein, a picornavirus protein, a flavivirus protein, a filovirus protein, a rhabdovirus protein, a togavirus protein, an arterivirus protein, a bunyavirus protein, an arenavirus protein, a reovirus protein, a bornavirus protein, a retrovirus protein, an adenovirus protein, a herpesvirus protein, a polyomavirus protein, a papillomavirus protein, a poxvirus protein, or a hepadnavirus protein.

25. (canceled)

26. The RNA molecule of claim 1, wherein the first antigenic protein is a SARS-CoV-2 spike glycoprotein or an influenza virus glycoprotein.

27. The RNA molecule of claim 26, wherein the SSARS-CoV-2 spike glycoprotein comprises an amino acid sequence having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to a sequence of SEQ ID NO:14, SEQ ID NO:15, SEQ ID NO:16, or SEQ ID NO:17.

28. The RNA molecule of claim 1, wherein the first transgene of the second polynucleotide comprises a sequence having at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to a sequence of SEQ ID NO:10, SEQ ID NO:11, SEQ ID NO:12, or SEQ ID NO:13.

29. The RNA molecule of claim 1, wherein the first transgene is expressed from a first subgenomic promoter.

30. (canceled)

31. (canceled)

32. (canceled)

33. (canceled)

34. (canceled)

35. The RNA molecule of claim 1, wherein the first polynucleotide is located 5′ of the second polynucleotide.

36. The RNA molecule of claim 35, further comprising an intergenic region located between the first polynucleotide and the second polynucleotide.

37. The RNA molecule of claim 36, wherein the intergenic region comprises a sequence having at least 85% identity to a sequence of SEQ ID NO:7.

38. The RNA molecule of claim 1, wherein the RNA molecule is a self-replicating RNA molecule.

39. The RNA molecule of claim 38, wherein the RNA molecule comprises a sequence having at least 80%, at least 85%, at least 90%, at least 91%, at least 92%, at least 93%, at least 94%, at least 95%, at least 96%, at least 97%, at least 97.5%, at least 98%, at least 98.5%, at least 99%, at least 99.5%, at least 99.6%, at least 99.7%, at least 99.8%, at least 99.9%, or 100% identity to
(i) a sequence of SEQ ID NO:1; or
(ii) a sequence of SEQ ID NO:2; or
(iii) a sequence of SEQ ID NO:3; or
(iv) a sequence of SEQ ID NO:4; or
(v) a sequence comprising a sequence of SEQ ID NO:5, SEQ ID NO:6, SEQ ID NO:10, and SEQ ID NO:9; or
(vi) a sequence comprising a sequence of SEQ ID NO:5, SEQ ID NO:6, SEQ ID NO: 11, and SEQ ID NO:9; or
(vii) a sequence comprising a sequence of SEQ ID NO:5, SEQ ID NO:6, SEQ ID NO: 12, and SEQ ID NO:9; or
(viii) a sequence comprising a sequence of SEQ ID NO:5, SEQ ID NO:6, SEQ ID NO: 13, and SEQ ID NO:9, wherein T is substituted with U.

40. The RNA molecule of claim 38, wherein the RNA molecule further comprises a 5′ cap.

41. The RNA molecule of claim 40, wherein the 5′ cap has a Cap 1 structure, a Cap 1 (^{m6}A) structure, a Cap 2 structure, or a Cap 0 structure.

42. A DNA molecule encoding the RNA molecule of claim 1.

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. A composition comprising the RNA molecule of claim 1 and a lipid formulation selected from a lipoplex, a liposome, a lipid nanoparticle, a polymer-based carrier, an exosome, a lamellar body, a micelle, and an emulsion.

87. (canceled)

88. (canceled)

89. (canceled)

90. (canceled)

91. The composition of claim 86, wherein
(i) the lipid formulation is a lipid nanoparticle having a size of less than about 200 nm; or
(ii) the lipid formulation encapsulates the nucleic acid molecule or is complexed to the nucleic acid molecule; or
(iii) the composition has a total lipid:nucleic acid molecule weight ratio of about 50:1 to about 10:1; or
(iv) any combination thereof.

92. (canceled)

93. (canceled)

94. (canceled)

95. (canceled)

96. (canceled)

97. (canceled)

98. The composition of claim 86, wherein the lipid formulation comprises an ionizable cationic lipid.

99. The composition of claim 98, wherein the ionizable cationic lipid has a structure of Formula I: 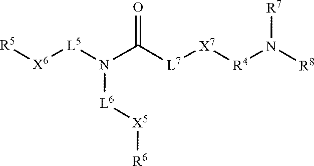
or a pharmaceutically acceptable salt or solvate thereof, wherein R⁵ and R⁶ are each independently selected from the group consisting of a linear or branched C₁₋C₃₁ alkyl, C₂₋ C₃₁ alkenyl or C₂₋C₃₁ alkynyl and cholesteryl; L⁵ and L⁶ are each independently selected from the group consisting of a linear C₁₋C₂₀ alkyl and C₂-C₂₀ alkenyl; X⁵ is —C(O)O—, whereby —C(O)O—R⁶ is formed or —OC(O)— whereby —OC(O)—R⁶ is formed; X⁶ is —C(O)O—whereby —C(O)O—R⁵ is formed or —OC(O)— whereby —OC(O)—R⁵ is formed; X⁷ is S or O; L⁷ is absent or lower alkyl; R⁴ is a linear or branched C₁₋C₆ alkyl; and R⁷ and R⁸ are each independently selected from the group consisting of a hydrogen and a linear or branched C₁₋C₆ alkyl.

100. (canceled)

101. The composition of claim 98, wherein the ionizable cationic lipid is ATX-126:

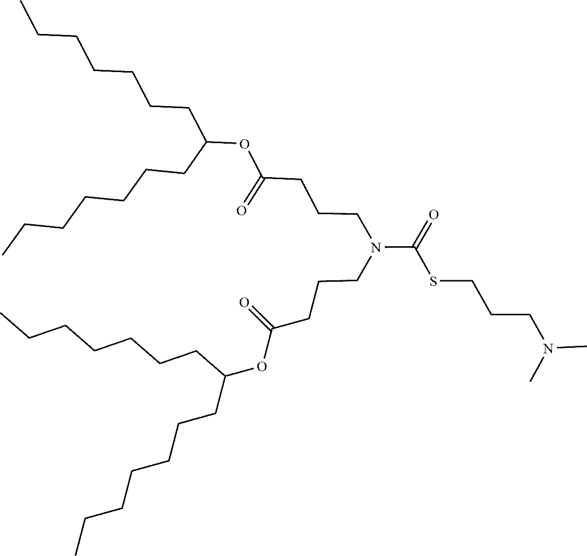 [SMILES: CCCCCCCC(CCCCCCC)OC(=O)CCCN(CCCC(=O)OC(CCCCCCC)CCCCCCC)C(=O)SCCCN(C)C]

102. (canceled)

103. (canceled)

104. The composition of claim 98, wherein the lipid formulation further comprises
(i) a helper lipid;
(ii) a helper lipid that is a phospholipid;
(iii) a polyethylene glycol (PEG)-lipid conjugate;
(iv) cholesterol; or
(v) any combination thereof.

105. (canceled)

106. The composition of claim 104, wherein
(A) the helper lipid is selected from dioleoylphosphatidyl ethanolamine (DOPE), dimyristoylphosphatidyl choline (DMPC), distearoylphosphatidyl choline (DSPC), dimyristoylphosphatidyl glycerol (DMPG), dipalmitoyl phosphatidylcholine (DPPC), and phosphatidylcholine (PC); or (B) the PEG-lipid conjugate is PEG-DMG; or
(C) both (A) and (B).

107. (canceled)

108. (canceled)

109. (canceled)

110. (canceled)

111. (canceled)

112. The composition of claim 86, wherein
(i) the lipid portion of the lipid formulation comprises about 40 mol% to about 60 mol% of the an ionizable cationic lipid, about 4 mol% to about 16 mol% DSPC, about 30 mol% to about 47 mol% cholesterol, and about 0.5 mol% to about 3 mol% PEG2000-DMG; or
(ii) the composition is a lyophilized composition; or
(iii) both (i) and (ii).

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. The composition of claim 86, wherein the RNA molecule comprises
(A) a sequence of SEQ ID NO:1; or
(B) a sequence of SEQ ID NO:2; or
(C) a sequence of SEQ ID NO:3; or
(D) a sequence of SEQ ID NO:4; or
(E) a sequence comprising a sequence of SEQ ID NO:5, SEQ ID NO:6, SEQ ID NO:10, and SEQ ID NO:9; or
(F) a sequence comprising a sequence of SEQ ID NO:5, SEQ ID NO:6, SEQ ID NO:11, and SEQ ID NO:9; or
(G) a sequence comprising a sequence of SEQ ID NO:5, SEQ ID NO:6, SEQ ID NO:12, and SEQ ID NO:9; or;
(H) a sequence comprising a sequence of SEQ ID NO:5, SEQ ID NO:6, SEQ ID NO:13, and SEQ ID NO:9,
wherein T is substituted with U.

140. (canceled)

141. (canceled)

142. (canceled)

143. A method of administering the composition of claim 86 to a subject in need thereof, wherein the composition is lyophilized and is reconstituted prior to administration.

144. A method of preventing or ameliorating COVID-19, comprising administering the composition of claim 86 to a subject in need thereof.

145. (canceled)

146. (canceled)

147. A method of administering a booster dose to a vaccinated subject, comprising administering the composition of claim 86 to a subject who was previously vaccinated against coronavirus.

148. The method of claim 147, wherein the composition is administered at a dosage of about 0.01 µg to about 1,000 µg of nucleic acid.

149. (canceled)

150. A method of inducing an immune response against a coronavirus in a subject comprising:
administering to the subject an effective amount of an RNA molecule of claim 1.

151. (canceled)

152. A method of inducing an immune response in a subject comprising:
administering to the subject an effective amount of a composition of claim 86.

153. (canceled)

154. (canceled)

155. (canceled)

156. (canceled)

157. (canceled)

158. (canceled)

159. (canceled)

160. (canceled)

161. (canceled)

162. (canceled)

163. (canceled)

164. (canceled)

165. (canceled)

166. (canceled)

167. (canceled)

168. (canceled)

169. (canceled)

170. (canceled)

171. (canceled)

172. (canceled)

173. (canceled)

174. (canceled)

175. (canceled)

176. (canceled)

177. (canceled)

178. (canceled)

179. (canceled)

180. (canceled)

181. (canceled)

182. (canceled)

183. (canceled)

184. (canceled)

185. (canceled)

186. (canceled)

187. (canceled)

188. (canceled)


SEQUENCE LISTING (320 sequences)

SEQ ID NO: 1 - DNA, 11860 bp, synthetic construct
  source 1..11860: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggccgccaccatgttcgtgttcctggtgctgctgcccctggtgtctagccagtgcgtgaacctgaccaccaggacccagctgcctcccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgccaaccccgtgctgcccttcaacgacggcgtgtacttcgccagcaccgagaagtccaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtactccagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccctatcaacctggtgaggggcctgccccagggcttcagcgccctggagcccctggtggacctgcccatcggcatcaacatcaccaggttccagaccctgctggccctgcacaggagctacctgacccctggcgacagcagctccggctggaccgccggcgccgccgcttactacgtgggctacctgcagcccaggaccttcctgctgaagtacaacgagaacggcaccatcaccgacgccgtggactgcgccctggaccctctgagcgagacaaagtgcaccctgaagtccttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtataacagcgccagcttcagcaccttcaagtgctacggcgtgagccccaccaagctgaacgacctgtgcttcaccaacgtgtacgccgacagcttcgtgatcaggggcgacgaggtgaggcagatcgcccctggccagaccggcaacatcgccgactacaactacaagctgcccgacgacttcaccggctgcgtgatcgcctggaacagcaacaacctggacagcaaggtgggcggcaactacaactacctgtaccggctgttcagaaagagcaacctgaagcccttcgagagggacatcagcaccgagatctaccaggccggcagcaccccttgcaacggcgtgaagggcttcaactgctacttccctctgcagagctacggcttccagcccacctacggcgtgggctaccagccctacagggtggtggtcctgagcttcgagctgctgcacgcccctgccaccgtgtgcggccccaagaagtccaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggaccctcagaccctggagatcctggacatcaccccttgcagcttcggcggcgtgagcgtgatcacccctggcaccaacaccagcaaccaggtggccgtgctgtaccagggcgtgaactgcaccgaggtgcccgtggccatccacgccgaccagctgacccctacctggagggtgtactccaccggcagcaacgtgttccagaccagggccggctgcctgatcggcgccgagcacgtgaacaacagctacgagtgcgacatccccatcggcgccggcatctgcgccagctaccagacccagaccaacagccccgggagcgccagcagcgtggccagccagagcatcatcgcctacaccatgagcctgggcgtggagaacagcgtggcctacagcaacaacagcatcgccatccccaccaacttcaccatcagcgtgaccaccgagatcctgcccgtgagcatgaccaagaccagcgtggactgcaccatgtatatctgcggcgacagcaccgagtgcagcaacctgctgctccagtacggcagcttctgcacccagctgaacagggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgcctcccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccacccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttccctcagagcgcccctcacggcgtggtgttcctgcacgtgacctacgtgcccgcccaggagaagaacttcaccacagcccctgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccctctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 11860 bp, synthetic construct
  source 1..11860: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggccgccaccatgttcgtgttcctggtgctgctgcccctggtgtctagccagtgcgtgaacctgaccaccaggacccagctgcctcccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagtccaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtactccagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccctatcaacctggtgagggacctgccccagggcttcagcgccctggagcccctggtggacctgcccatcggcatcaacatcaccaggttccagaccctgctggccctgcacaggagctacctgacccctggcgacagcagctccggctggaccgccggcgccgccgcttactacgtgggctacctgcagcccaggaccttcctgctgaagtacaacgagaacggcaccatcaccgacgccgtggactgcgccctggaccctctgagcgagacaaagtgcaccctgaagtccttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtataacagcgccagcttcagcaccttcaagtgctacggcgtgagccccaccaagctgaacgacctgtgcttcaccaacgtgtacgccgacagcttcgtgatcaggggcgacgaggtgaggcagatcgcccctggccagaccggcaagatcgccgactacaactacaagctgcccgacgacttcaccggctgcgtgatcgcctggaacagcaacaacctggacagcaaggtgggcggcaactacaactacctgtaccggctgttcagaaagagcaacctgaagcccttcgagagggacatcagcaccgagatctaccaggccggcagcaccccttgcaacggcgtggagggcttcaactgctacttccctctgcagagctacggcttccagcccaccaacggcgtgggctaccagccctacagggtggtggtcctgagcttcgagctgctgcacgcccctgccaccgtgtgcggccccaagaagtccaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggaccctcagaccctggagatcctggacatcaccccttgcagcttcggcggcgtgagcgtgatcacccctggcaccaacaccagcaaccaggtggccgtgctgtaccagggcgtgaactgcaccgaggtgcccgtggccatccacgccgaccagctgacccctacctggagggtgtactccaccggcagcaacgtgttccagaccagggccggctgcctgatcggcgccgagcacgtgaacaacagctacgagtgcgacatccccatcggcgccggcatctgcgccagctaccagacccagaccaacagccccgggagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtatatctgcggcgacagcaccgagtgcagcaacctgctgctccagtacggcagcttctgcacccagctgaacagggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgcctcccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccacccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttccctcagagcgcccctcacggcgtggtgttcctgcacgtgacctacgtgcccgcccaggagaagaacttcaccacagcccctgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccctctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - DNA, 11851 bp, synthetic construct
  source 1..11851: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggccgccaccatgttcgtgttcctggtgctgctgcccctggtgtctagccagtgcgtgaacctgaccaccaggacccagctgcctcccgcctacaccaacagcttcaccaggggcgtgtactaccccgacaaggtgttcaggagcagcgtgctgcacagcacccaggacctgttcctgcccttcttcagcaacgtgacctggttccacgccatcagcggcaccaacggcaccaagaggttcgacaaccccgtgctgcccttcaacgacggcgtgtacttcgccagcaccgagaagtccaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtaccacaagaacaacaagagctggatggagagcgagttcagggtgtactccagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccctatcaacctggtgagggacctgccccagggcttcagcgccctggagcccctggtggacctgcccatcggcatcaacatcaccaggttccagaccctgctggccctgcacaggagctacctgacccctggcgacagcagctccggctggaccgccggcgccgccgcttactacgtgggctacctgcagcccaggaccttcctgctgaagtacaacgagaacggcaccatcaccgacgccgtggactgcgccctggaccctctgagcgagacaaagtgcaccctgaagtccttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtataacagcgccagcttcagcaccttcaagtgctacggcgtgagccccaccaagctgaacgacctgtgcttcaccaacgtgtacgccgacagcttcgtgatcaggggcgacgaggtgaggcagatcgcccctggccagaccggcaagatcgccgactacaactacaagctgcccgacgacttcaccggctgcgtgatcgcctggaacagcaacaacctggacagcaaggtgggcggcaactacaactacctgtaccggctgttcagaaagagcaacctgaagcccttcgagagggacatcagcaccgagatctaccaggccggcagcaccccttgcaacggcgtggagggcttcaactgctacttccctctgcagagctacggcttccagcccacctacggcgtgggctaccagccctacagggtggtggtcctgagcttcgagctgctgcacgcccctgccaccgtgtgcggccccaagaagtccaccaacctggtgaagaacaagtgcgtgaacttcaacttcaacggcctgaccggcaccggcgtgctgaccgagagcaacaagaagttcctgcccttccagcagttcggcagggacatcgacgacaccaccgacgccgtgagggaccctcagaccctggagatcctggacatcaccccttgcagcttcggcggcgtgagcgtgatcacccctggcaccaacaccagcaaccaggtggccgtgctgtaccagggcgtgaactgcaccgaggtgcccgtggccatccacgccgaccagctgacccctacctggagggtgtactccaccggcagcaacgtgttccagaccagggccggctgcctgatcggcgccgagcacgtgaacaacagctacgagtgcgacatccccatcggcgccggcatctgcgccagctaccagacccagaccaacagccacgggagcgccagcagcgtggccagccagagcatcatcgcctacaccatgagcctgggcgccgagaacagcgtggcctacagcaacaacagcatcgccatccccatcaacttcaccatcagcgtgaccaccgagatcctgcccgtgagcatgaccaagaccagcgtggactgcaccatgtatatctgcggcgacagcaccgagtgcagcaacctgctgctccagtacggcagcttctgcacccagctgaacagggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgcctcccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctggccaggctggacccacccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttccctcagagcgcccctcacggcgtggtgttcctgcacgtgacctacgtgcccgcccaggagaagaacttcaccacagcccctgccatctgccacgacggcaaggcccacttccccagggagggcgtgttcgtgagcaacggcacccactggttcgtgacccagaggaacttctacgagccccagatcatcaccacccacaacaccttcgtgagcggcaactgcgacgtggtgatcggcatcgtgaacaacaccgtgtacgaccctctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - DNA, 11860 bp, synthetic construct
  source 1..11860: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggccgccaccatgttcgtgttcctggtgctgctgcccctggtgtctagccagtgcgtgaacttcaccaacaggacccagctgcctagcgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagtccaacatcatcaggggctggatcttcggcaccaccctggacagcaagacccagagcctgctgatcgtgaacaacgccaccaacgtggtgatcaaggtgtgcgagttccagttctgcaactaccccttcctgggcgtgtactaccacaagaacaacaagagctggatggagagcgagttcagggtgtactccagcgccaacaactgcaccttcgagtacgtgagccagcccttcctgatggacctggagggcaagcagggcaacttcaagaacctgagcgagttcgtgttcaagaacatcgacggctacttcaagatctacagcaagcacacccctatcaacctggtgagggacctgccccagggcttcagcgccctggagcccctggtggacctgcccatcggcatcaacatcaccaggttccagaccctgctggccctgcacaggagctacctgacccctggcgacagcagctccggctggaccgccggcgccgccgcttactacgtgggctacctgcagcccaggaccttcctgctgaagtacaacgagaacggcaccatcaccgacgccgtggactgcgccctggaccctctgagcgagacaaagtgcaccctgaagtccttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtataacagcgccagcttcagcaccttcaagtgctacggcgtgagccccaccaagctgaacgacctgtgcttcaccaacgtgtacgccgacagcttcgtgatcaggggcgacgaggtgaggcagatcgcccctggccagaccggcaccatcgccgactacaactacaagctgcccgacgacttcaccggctgcgtgatcgcctggaacagcaacaacctggacagcaaggtgggcggcaactacaactacctgtaccggctgttcagaaagagcaacctgaagcccttcgagagggacatcagcaccgagatctaccaggccggcagcaccccttgcaacggcgtgaagggcttcaactgctacttccctctgcagagctacggcttccagcccacctacggcgtgggctaccagccctacagggtggtggtcctgagcttcgagctgctgcacgcccctgccaccgtgtgcggccccaagaagtccaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggaccctcagaccctggagatcctggacatcaccccttgcagcttcggcggcgtgagcgtgatcacccctggcaccaacaccagcaaccaggtggccgtgctgtaccagggcgtgaactgcaccgaggtgcccgtggccatccacgccgaccagctgacccctacctggagggtgtactccaccggcagcaacgtgttccagaccagggccggctgcctgatcggcgccgagtacgtgaacaacagctacgagtgcgacatccccatcggcgccggcatctgcgccagctaccagacccagaccaacagccccgggagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtatatctgcggcgacagcaccgagtgcagcaacctgctgctccagtacggcagcttctgcacccagctgaacagggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgcctcccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccacccgaggccgaggtgcagatcgacaggctgatcaccggcaggctgcagagcctgcagacctacgtgacccagcagctgatcagggccgccgagatcagggccagcgccaacctggccgccatcaagatgagcgagtgcgtgctgggccagagcaagagggtggacttctgcggcaagggctaccacctgatgagcttccctcagagcgcccctcacggcgtggtgttcctgcacgtgacctacgtgcccgcccaggagaagaacttcaccacagcccctgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccctctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - DNA, 44 bp, synthetic construct
  source 1..44: mol_type = other DNA
atgggcggcgcatgagagaagcccagaccaattacctacccaaa

SEQ ID NO: 6 - DNA, 7482 bp, synthetic construct
  source 1..7482: mol_type = other DNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaa

SEQ ID NO: 7 - DNA, 44 bp, synthetic construct
  source 1..44: mol_type = other DNA
cctgaatggactacgacatagtctagtccgccaaggccgccacc

SEQ ID NO: 8 - DNA, 468 bp, synthetic construct
  source 1..468: mol_type = other DNA
actcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - DNA, 338 bp, synthetic construct
  source 1..338: mol_type = other DNA
actcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttc

SEQ ID NO: 10 - DNA, 3822 bp, synthetic construct
  source 1..3822: mol_type = other DNA
atgttcgtgttcctggtgctgctgcccctggtgtctagccagtgcgtgaacctgaccaccaggacccagctgcctcccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgccaaccccgtgctgcccttcaacgacggcgtgtacttcgccagcaccgagaagtccaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtactccagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccctatcaacctggtgaggggcctgccccagggcttcagcgccctggagcccctggtggacctgcccatcggcatcaacatcaccaggttccagaccctgctggccctgcacaggagctacctgacccctggcgacagcagctccggctggaccgccggcgccgccgcttactacgtgggctacctgcagcccaggaccttcctgctgaagtacaacgagaacggcaccatcaccgacgccgtggactgcgccctggaccctctgagcgagacaaagtgcaccctgaagtccttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtataacagcgccagcttcagcaccttcaagtgctacggcgtgagccccaccaagctgaacgacctgtgcttcaccaacgtgtacgccgacagcttcgtgatcaggggcgacgaggtgaggcagatcgcccctggccagaccggcaacatcgccgactacaactacaagctgcccgacgacttcaccggctgcgtgatcgcctggaacagcaacaacctggacagcaaggtgggcggcaactacaactacctgtaccggctgttcagaaagagcaacctgaagcccttcgagagggacatcagcaccgagatctaccaggccggcagcaccccttgcaacggcgtgaagggcttcaactgctacttccctctgcagagctacggcttccagcccacctacggcgtgggctaccagccctacagggtggtggtcctgagcttcgagctgctgcacgcccctgccaccgtgtgcggccccaagaagtccaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggaccctcagaccctggagatcctggacatcaccccttgcagcttcggcggcgtgagcgtgatcacccctggcaccaacaccagcaaccaggtggccgtgctgtaccagggcgtgaactgcaccgaggtgcccgtggccatccacgccgaccagctgacccctacctggagggtgtactccaccggcagcaacgtgttccagaccagggccggctgcctgatcggcgccgagcacgtgaacaacagctacgagtgcgacatccccatcggcgccggcatctgcgccagctaccagacccagaccaacagccccgggagcgccagcagcgtggccagccagagcatcatcgcctacaccatgagcctgggcgtggagaacagcgtggcctacagcaacaacagcatcgccatccccaccaacttcaccatcagcgtgaccaccgagatcctgcccgtgagcatgaccaagaccagcgtggactgcaccatgtatatctgcggcgacagcaccgagtgcagcaacctgctgctccagtacggcagcttctgcacccagctgaacagggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgcctcccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccacccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttccctcagagcgcccctcacggcgtggtgttcctgcacgtgacctacgtgcccgcccaggagaagaacttcaccacagcccctgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccctctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 11 - DNA, 3822 bp, synthetic construct
  source 1..3822: mol_type = other DNA
atgttcgtgttcctggtgctgctgcccctggtgtctagccagtgcgtgaacctgaccaccaggacccagctgcctcccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagtccaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtactccagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccctatcaacctggtgagggacctgccccagggcttcagcgccctggagcccctggtggacctgcccatcggcatcaacatcaccaggttccagaccctgctggccctgcacaggagctacctgacccctggcgacagcagctccggctggaccgccggcgccgccgcttactacgtgggctacctgcagcccaggaccttcctgctgaagtacaacgagaacggcaccatcaccgacgccgtggactgcgccctggaccctctgagcgagacaaagtgcaccctgaagtccttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtataacagcgccagcttcagcaccttcaagtgctacggcgtgagccccaccaagctgaacgacctgtgcttcaccaacgtgtacgccgacagcttcgtgatcaggggcgacgaggtgaggcagatcgcccctggccagaccggcaagatcgccgactacaactacaagctgcccgacgacttcaccggctgcgtgatcgcctggaacagcaacaacctggacagcaaggtgggcggcaactacaactacctgtaccggctgttcagaaagagcaacctgaagcccttcgagagggacatcagcaccgagatctaccaggccggcagcaccccttgcaacggcgtggagggcttcaactgctacttccctctgcagagctacggcttccagcccaccaacggcgtgggctaccagccctacagggtggtggtcctgagcttcgagctgctgcacgcccctgccaccgtgtgcggccccaagaagtccaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggaccctcagaccctggagatcctggacatcaccccttgcagcttcggcggcgtgagcgtgatcacccctggcaccaacaccagcaaccaggtggccgtgctgtaccagggcgtgaactgcaccgaggtgcccgtggccatccacgccgaccagctgacccctacctggagggtgtactccaccggcagcaacgtgttccagaccagggccggctgcctgatcggcgccgagcacgtgaacaacagctacgagtgcgacatccccatcggcgccggcatctgcgccagctaccagacccagaccaacagccccgggagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtatatctgcggcgacagcaccgagtgcagcaacctgctgctccagtacggcagcttctgcacccagctgaacagggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgcctcccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccacccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttccctcagagcgcccctcacggcgtggtgttcctgcacgtgacctacgtgcccgcccaggagaagaacttcaccacagcccctgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccctctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 12 - DNA, 3813 bp, synthetic construct
  source 1..3813: mol_type = other DNA
atgttcgtgttcctggtgctgctgcccctggtgtctagccagtgcgtgaacctgaccaccaggacccagctgcctcccgcctacaccaacagcttcaccaggggcgtgtactaccccgacaaggtgttcaggagcagcgtgctgcacagcacccaggacctgttcctgcccttcttcagcaacgtgacctggttccacgccatcagcggcaccaacggcaccaagaggttcgacaaccccgtgctgcccttcaacgacggcgtgtacttcgccagcaccgagaagtccaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtaccacaagaacaacaagagctggatggagagcgagttcagggtgtactccagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccctatcaacctggtgagggacctgccccagggcttcagcgccctggagcccctggtggacctgcccatcggcatcaacatcaccaggttccagaccctgctggccctgcacaggagctacctgacccctggcgacagcagctccggctggaccgccggcgccgccgcttactacgtgggctacctgcagcccaggaccttcctgctgaagtacaacgagaacggcaccatcaccgacgccgtggactgcgccctggaccctctgagcgagacaaagtgcaccctgaagtccttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtataacagcgccagcttcagcaccttcaagtgctacggcgtgagccccaccaagctgaacgacctgtgcttcaccaacgtgtacgccgacagcttcgtgatcaggggcgacgaggtgaggcagatcgcccctggccagaccggcaagatcgccgactacaactacaagctgcccgacgacttcaccggctgcgtgatcgcctggaacagcaacaacctggacagcaaggtgggcggcaactacaactacctgtaccggctgttcagaaagagcaacctgaagcccttcgagagggacatcagcaccgagatctaccaggccggcagcaccccttgcaacggcgtggagggcttcaactgctacttccctctgcagagctacggcttccagcccacctacggcgtgggctaccagccctacagggtggtggtcctgagcttcgagctgctgcacgcccctgccaccgtgtgcggccccaagaagtccaccaacctggtgaagaacaagtgcgtgaacttcaacttcaacggcctgaccggcaccggcgtgctgaccgagagcaacaagaagttcctgcccttccagcagttcggcagggacatcgacgacaccaccgacgccgtgagggaccctcagaccctggagatcctggacatcaccccttgcagcttcggcggcgtgagcgtgatcacccctggcaccaacaccagcaaccaggtggccgtgctgtaccagggcgtgaactgcaccgaggtgcccgtggccatccacgccgaccagctgacccctacctggagggtgtactccaccggcagcaacgtgttccagaccagggccggctgcctgatcggcgccgagcacgtgaacaacagctacgagtgcgacatccccatcggcgccggcatctgcgccagctaccagacccagaccaacagccacgggagcgccagcagcgtggccagccagagcatcatcgcctacaccatgagcctgggcgccgagaacagcgtggcctacagcaacaacagcatcgccatccccatcaacttcaccatcagcgtgaccaccgagatcctgcccgtgagcatgaccaagaccagcgtggactgcaccatgtatatctgcggcgacagcaccgagtgcagcaacctgctgctccagtacggcagcttctgcacccagctgaacagggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgcctcccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctggccaggctggacccacccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttccctcagagcgcccctcacggcgtggtgttcctgcacgtgacctacgtgcccgcccaggagaagaacttcaccacagcccctgccatctgccacgacggcaaggcccacttccccagggagggcgtgttcgtgagcaacggcacccactggttcgtgacccagaggaacttctacgagccccagatcatcaccacccacaacaccttcgtgagcggcaactgcgacgtggtgatcggcatcgtgaacaacaccgtgtacgaccctctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 13 - DNA, 3822 bp, synthetic construct
  source 1..3822: mol_type = other DNA
atgttcgtgttcctggtgctgctgcccctggtgtctagccagtgcgtgaacttcaccaacaggacccagctgcctagcgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagtccaacatcatcaggggctggatcttcggcaccaccctggacagcaagacccagagcctgctgatcgtgaacaacgccaccaacgtggtgatcaaggtgtgcgagttccagttctgcaactaccccttcctgggcgtgtactaccacaagaacaacaagagctggatggagagcgagttcagggtgtactccagcgccaacaactgcaccttcgagtacgtgagccagcccttcctgatggacctggagggcaagcagggcaacttcaagaacctgagcgagttcgtgttcaagaacatcgacggctacttcaagatctacagcaagcacacccctatcaacctggtgagggacctgccccagggcttcagcgccctggagcccctggtggacctgcccatcggcatcaacatcaccaggttccagaccctgctggccctgcacaggagctacctgacccctggcgacagcagctccggctggaccgccggcgccgccgcttactacgtgggctacctgcagcccaggaccttcctgctgaagtacaacgagaacggcaccatcaccgacgccgtggactgcgccctggaccctctgagcgagacaaagtgcaccctgaagtccttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtataacagcgccagcttcagcaccttcaagtgctacggcgtgagccccaccaagctgaacgacctgtgcttcaccaacgtgtacgccgacagcttcgtgatcaggggcgacgaggtgaggcagatcgcccctggccagaccggcaccatcgccgactacaactacaagctgcccgacgacttcaccggctgcgtgatcgcctggaacagcaacaacctggacagcaaggtgggcggcaactacaactacctgtaccggctgttcagaaagagcaacctgaagcccttcgagagggacatcagcaccgagatctaccaggccggcagcaccccttgcaacggcgtgaagggcttcaactgctacttccctctgcagagctacggcttccagcccacctacggcgtgggctaccagccctacagggtggtggtcctgagcttcgagctgctgcacgcccctgccaccgtgtgcggccccaagaagtccaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggaccctcagaccctggagatcctggacatcaccccttgcagcttcggcggcgtgagcgtgatcacccctggcaccaacaccagcaaccaggtggccgtgctgtaccagggcgtgaactgcaccgaggtgcccgtggccatccacgccgaccagctgacccctacctggagggtgtactccaccggcagcaacgtgttccagaccagggccggctgcctgatcggcgccgagtacgtgaacaacagctacgagtgcgacatccccatcggcgccggcatctgcgccagctaccagacccagaccaacagccccgggagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtatatctgcggcgacagcaccgagtgcagcaacctgctgctccagtacggcagcttctgcacccagctgaacagggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgcctcccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccacccgaggccgaggtgcagatcgacaggctgatcaccggcaggctgcagagcctgcagacctacgtgacccagcagctgatcagggccgccgagatcagggccagcgccaacctggccgccatcaagatgagcgagtgcgtgctgggccagagcaagagggtggacttctgcggcaagggctaccacctgatgagcttccctcagagcgcccctcacggcgtggtgttcctgcacgtgacctacgtgcccgcccaggagaagaacttcaccacagcccctgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccctctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaa

SEQ ID NO: 14 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 16 - protein, 1270 aa, synthetic construct
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHGSASSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - protein, 1273 aa, synthetic construct
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - DNA, 11860 bp, synthetic construct
  source 1..11860: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccatgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagtccaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagacgggcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - DNA, 44 bp, synthetic construct; identical to SEQ ID NO: 5
  source 1..44: mol_type = other DNA

SEQ ID NO: 20 - DNA, 7482 bp, synthetic construct
  source 1..7482: mol_type = other DNA
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaa

SEQ ID NO: 21 - DNA, 44 bp, synthetic construct; identical to SEQ ID NO: 7
  source 1..44: mol_type = other DNA

SEQ ID NO: 22 - DNA, 468 bp, synthetic construct; identical to SEQ ID NO: 8
  source 1..468: mol_type = other DNA

SEQ ID NO: 23 - DNA, 338 bp, synthetic construct; identical to SEQ ID NO: 9
  source 1..338: mol_type = other DNA

SEQ ID NO: 24 - DNA, 3822 bp, synthetic construct
  source 1..3822: mol_type = other DNA
atgttcgtcttcctggtcctgctgcctctggtctcctcacagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagtccaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagacgggcccggagcgtggcctcccagtctatcatcgcctataccatgtccctgggcgccgagaacagcgtggcctactctaacaatagcatcgccatcccaaccaacttcacaatctctgtgaccacagagatcctgcccgtgtccatgaccaagacatctgtggactgcacaatgtatatctgtggcgattctaccgagtgcagcaacctgctgctccagtacggcagcttttgtacccagctgaatagagccctgacaggcatcgccgtggagcaggataagaacacacaggaggtgttcgcccaggtgaagcaaatctacaagaccccccctatcaaggactttggcggcttcaatttttcccagatcctgcctgatccatccaagccttctaagcggagctttatcgaggacctgctgttcaacaaggtgaccctggccgatgccggcttcatcaagcagtatggcgattgcctgggcgacatcgcagccagggacctgatctgcgcccagaagtttaatggcctgaccgtgctgccacccctgctgacagatgagatgatcgcacagtacacaagcgccctgctggccggcaccatcacatccggatggaccttcggcgcaggagccgccctccagatcccctttgccatgcagatggcctataggttcaacggcatcggcgtgacccagaatgtgctgtacgagaaccagaagctgatcgccaatcagtttaactccgccatcggcaagatccaggacagcctgtcctctacagccagcgccctgggcaagctccaggatgtggtgaatcagaacgcccaggccctgaataccctggtgaagcagctgagcagcaacttcggcgccatctctagcgtgctgaatgacatcctgagccggctggacaaggtggaggcagaggtgcagatcgaccggctgatcaccggccggctccagagcctccagacctatgtgacacagcagctgatcagggccgccgagatcagggccagcgccaatctggcagcaaccaagatgtccgagtgcgtgctgggccagtctaagagagtggacttttgtggcaagggctatcacctgatgtccttccctcagtctgccccacacggcgtggtgtttctgcacgtgacctacgtgcccgcccaggagaagaacttcaccacagcccctgccatctgccacgatggcaaggcccactttccaagggagggcgtgttcgtgtccaacggcacccactggtttgtgacacagcgcaatttctacgagccccagatcatcaccacagacaacaccttcgtgagcggcaactgtgacgtggtcatcggcatcgtgaacaataccgtgtatgatccactccagcccgagctggacagctttaaggaggagctggataagtatttcaagaatcacacctcccctgacgtggatctgggcgacatcagcggcatcaatgcctccgtggtgaacatccagaaggagatcgaccgcctgaacgaggtggctaagaatctgaacgagagcctgatcgacctccaggagctgggcaagtatgagcagtacatcaagtggccctggtacatctggctgggcttcatcgccggcctgatcgccatcgtgatggtgaccatcatgctgtgctgtatgacatcctgctgttcttgcctgaagggctgctgtagctgtggctcctgctgtaagtttgacgaggatgactctgaacctgtgctgaagggcgtgaagctgcattacacctaa

SEQ ID NO: 25 - DNA, 3822 bp, synthetic construct
  source 1..3822: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 26 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - DNA, 8038 bp, synthetic construct
  source 1..8038: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagaccgtgctgaccgccaggaacatcggcctgtgcagcagcgacgtgatggagaggagccggagaggcatgagcatcctgaggaagaaatacctgaagcccagcaacaacgtgctgttcagcgtgggcagcaccatctaccacgagaagagggacctgctcaggagctggcacctgcccagcgtgttccacctgaggggcaagcagaactacacctgcaggtgcgagaccatcgtgagctgcgacggctacgtggtgaagaggatcgccatcagccccggcctgtacggcaagcccagcggctacgccgctacaatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctcgtgggcctgaaccagaggatcgtggtcaacggcaggacccagaggaacaccaacacaatgaagaactacctgctgcccgtggtggcccaggctttcgccaggtgggccaaggagtacaaggaggaccaggaagacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggaacacaaggagcccagcccactgatcaccgccgaggacgtgcaggaggccaagtgcgctgccgacgaggccaaggaggtgagggaggccgaggaactgagggccgccctgccacccctggctgccgacgtggaggaacccaccctggaagccgacgtggacctgatgctgcaggaggccggcgccggaagcgtggagacacccaggggcctgatcaaggtgaccagctacgacggcgaggacaagatcggcagctacgccgtgctgagcccacaggccgtgctgaagtccgagaagctgagctgcatccacccactggccgagcaggtgatcgtgatcacccacagcggcaggaagggcaggtacgccgtggagccctaccacggcaaggtggtcgtgcccgagggccacgccatccccgtgcaggacttccaggccctgagcgagagcgccaccatcgtgtacaacgagagggagttcgtgaacaggtacctgcaccatatcgccacccacggcggagccctgaacaccgacgaggaatactacaagaccgtgaagcccagcgagcacgacggcgagtacctgtacgacatcgacaggaagcagtgcgtgaagaaagagctggtgaccggcctgggactgaccggcgagctggtggacccacccttccacgagttcgcctacgagagcctgaggaccagacccgccgctccctaccaggtgcccaccatcggcgtgtacggcgtgcccggcagcggaaagagcggcatcatcaagagcgccgtgaccaagaaagacctggtggtcagcgccaagaaagagaactgcgccgagatcatcagggacgtgaagaagatgaaaggcctggacgtgaacgcgcgcaccgtggacagcgtgctgctgaacggctgcaagcaccccgtggagaccctgtacatcgacgaggccttcgcttgccacgccggcaccctgagggccctgatcgccatcatcaggcccaagaaagccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtcgtgagcaccctgttctacgacaagaaaatgaggaccaccaaccccaaggagaccaaaatcgtgatcgacaccacaggcagcaccaagcccaagcaggacgacctgatcctgacctgcttcaggggctgggtgaagcagctgcagatcgactacaagggcaacgagatcatgaccgccgctgccagccagggcctgaccaggaagggcgtgtacgccgtgaggtacaaggtgaacgagaacccactgtacgctcccaccagcgagcacgtgaacgtgctgctgaccaggaccgaggacaggatcgtgtggaagaccctggccggcgacccctggatcaagaccctgaccgccaagtaccccggcaacttcaccgccaccatcgaagagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccacagagcagtggaacaccgtggactacttcgagaccgacaaggcccacagcgccgagatcgtgctgaaccagctgtgcgtgaggttcttcggcctggacctggacagcggcctgttcagcgcccccaccgtgccactgagcatcaggaacaaccactgggacaacagccccagcccaaacatgtacggcctgaacaaggaggtggtcaggcagctgagcaggcggtacccacagctgcccagggccgtggccaccggcagggtgtacgacatgaacaccggcaccctgaggaactacgaccccaggatcaacctggtgcccgtgaacaggcggctgccccacgccctggtgctgcaccacaacgagcacccacagagcgacttcagctccttcgtgagcaagctgaaaggcaggaccgtgctggtcgtgggcgagaagctgagcgtgcccggcaagatggtggactggctgagcgacaggcccgaggccaccttccgggccaggctggacctcggcatccccggcgacgtgcccaagtacgacatcatcttcgtgaacgtcaggaccccatacaagtaccaccattaccagcagtgcgaggaccacgccatcaagctgagcatgctgaccaagaaggcctgcctgcacctgaaccccggaggcacctgcgtgagcatcggctacggctacgccgacagggccagcgagagcatcattggcgccatcgccaggctgttcaagttcagcagggtgtgcaaacccaagagcagcctggaggaaaccgaggtgctgttcgtgttcatcggctacgaccggaaggccaggacccacaacccctacaagctgagcagcaccctgacaaacatctacaccggcagcaggctgcacgaggccggctgcgcccccagctaccacgtggtcaggggcgatatcgccaccgccaccgagggcgtgatcatcaacgctgccaacagcaagggccagcccggaggcggagtgtgcggcgccctgtacaagaagttccccgagagcttcgacctgcagcccatcgaggtgggcaaggccaggctggtgaagggcgccgctaagcacatcatccacgccgtgggccccaacttcaacaaggtgagcgaggtggaaggcgacaagcagctggccgaagcctacgagagcatcgccaagatcgtgaacgacaataactacaagagcgtggccatcccactgctcagcaccggcatcttcagcggcaacaaggacaggctgacccagagcctgaaccacctgctcaccgccctggacaccaccgatgccgacgtggccatctactgcagggacaagaagtgggagatgaccctgaaggaggccgtggccaggcgggaggccgtggaagagatctgcatcagcgacgactccagcgtgaccgagcccgacgccgagctggtgagggtgcaccccaagagctccctggccggcaggaagggctacagcaccagcgacggcaagaccttcagctacctggagggcaccaagttccaccaggccgctaaggacatcgccgagatcaacgctatgtggcccgtggccaccgaggccaacgagcaggtgtgcatgtacatcctgggcgagagcatgtccagcatcaggagcaagtgccccgtggaggaaagcgaggccagcacaccacccagcaccctgccctgcctgtgcatccacgctatgacacccgagagggtgcagcggctgaaggccagcaggcccgagcagatcaccgtgtgcagctccttcccactgcccaagtacaggatcaccggcgtgcagaagatccagtgcagccagcccatcctgttcagcccaaaggtgcccgcctacatccaccccaggaagtacctggtggagaccccacccgtggacgagacacccgagccaagcgccgagaaccagagcaccgagggcacacccgagcagccacccctgatcaccgaggacgagacaaggacccggaccccagagcccatcattatcgaggaagaggaagaggacagcatcagcctgctgagcgacggccccacccaccaggtgctgcaggtggaggccgacatccacggcccacccagcgtgtccagctccagctggagcatcccacacgccagcgacttcgacgtggacagcctgagcatcctggacaccctggagggcgccagcgtgacctccggcgccaccagcgccgagaccaacagctacttcgccaagagcatggagttcctggccaggcccgtgccagctcccaggaccgtgttcaggaacccaccccacccagctcccaggaccaggaccccaagcctggctcccagcagggcctgcagcaggaccagcctggtgagcaccccacccggcgtgaacagggtgatcaccagggaggaactggaggccctgacacccagcaggacccccagcaggtccgtgagcaggactagtctggtgtccaacccacccggcgtgaacagggtgatcaccagggaggaattcgaggccttcgtggcccagcaacagagacggttcgacgccggcgcctacatcttcagcagcgacaccggccagggacacctgcagcaaaagagcgtgaggcagaccgtgctgagcgaggtggtgctggagaggaccgagctggaaatcagctacgcccccaggctggaccaggagaaggaggaactgctcaggaagaaactgcagctgaaccccaccccagccaacaggagcaggtaccagagcaggaaggtggagaacatgaaggccatcaccgccaggcggatcctgcagggcctgggacactacctgaaggccgagggcaaggtggagtgctacaggaccctgcaccccgtgccactgtacagctccagcgtgaacagggccttctccagccccaaggtggccgtggaggcctgcaacgctatgctgaaggagaacttccccaccgtggccagctactgcatcatccccgagtacgacgcctacctggacatggtggacggcgccagctgctgcctggacaccgccagcttctgccccgccaagctgaggagcttccccaagaaacacagctacctggagcccaccatcaggagcgccgtgcccagcgccatccagaacaccctgcagaacgtgctggccgctgccaccaagaggaactgcaacgtgacccagatgagggagctgcccgtgctggacagcgctgccttcaacgtggagtgcttcaagaaatacgcctgcaacaacgagtactgggagaccttcaaggagaaccccatcaggctgaccgaagagaacgtggtgaactacatcaccaagctgaagggccccaaggccgctgccctgttcgctaagacccacaacctgaacatgctgcaggacatcccaatggacaggttcgtgatggacctgaagagggacgtgaaggtgacacccggcaccaagcacaccgaggagaggcccaaggtgcaggtgatccaggccgctgacccactggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagaccgacatcgccagcttcgacaagagcgaggatgacgctatggccctgaccgctctgatgatcctggaggacctgggcgtggacgccgagctgctcaccctgatcgaggctgccttcggcgagatcagctccatccacctgcccaccaagaccaagttcaagttcggcgctatgatgaaaagcggaatgttcctgaccctgttcgtgaacaccgtgatcaacattgtgatcgccagcagggtgctgcgggagaggctgaccggcagcccctgcgctgccttcatcggcgacgacaacatcgtgaagggcgtgaaaagcgacaagctgatggccgacaggtgcgccacctggctgaacatggaggtgaagatcatcgacgccgtggtgggcgagaaggccccctacttctgcggcggattcatcctgtgcgacagcgtgaccggcaccgcctgcagggtggccgaccccctgaagaggctgttcaagctgggcaagccactggccgctgacgatgagcacgacgatgacaggcggagggccctgcacgaggaaagcaccaggtggaacagggtgggcatcctgagcgagctgtgcaaggccgtggagagcaggtacgagaccgtgggcaccagcatcatcgtgatggctatgaccacactggccagctccgtcaagagcttctcctacctgaggggggcccctataactctctacggctaacctgaatggactacgacatagtctagtccgccaaggccgccaccactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 28 - DNA, 8038 bp, synthetic construct; identical to SEQ ID NO: 27
  source 1..8038: mol_type = other DNA

SEQ ID NO: 29 - DNA, 4238 bp, synthetic construct
  source 1..4238: mol_type = other DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatgttcgtgttcctggtgctgctgcccctggtgtctagccagtgcgtgaacctgaccaccaggacccagctgcctcccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgccaaccccgtgctgcccttcaacgacggcgtgtacttcgccagcaccgagaagtccaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtactccagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccctatcaacctggtgaggggcctgccccagggcttcagcgccctggagcccctggtggacctgcccatcggcatcaacatcaccaggttccagaccctgctggccctgcacaggagctacctgacccctggcgacagcagctccggctggaccgccggcgccgccgcttactacgtgggctacctgcagcccaggaccttcctgctgaagtacaacgagaacggcaccatcaccgacgccgtggactgcgccctggaccctctgagcgagacaaagtgcaccctgaagtccttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtataacagcgccagcttcagcaccttcaagtgctacggcgtgagccccaccaagctgaacgacctgtgcttcaccaacgtgtacgccgacagcttcgtgatcaggggcgacgaggtgaggcagatcgcccctggccagaccggcaacatcgccgactacaactacaagctgcccgacgacttcaccggctgcgtgatcgcctggaacagcaacaacctggacagcaaggtgggcggcaactacaactacctgtaccggctgttcagaaagagcaacctgaagcccttcgagagggacatcagcaccgagatctaccaggccggcagcaccccttgcaacggcgtgaagggcttcaactgctacttccctctgcagagctacggcttccagcccacctacggcgtgggctaccagccctacagggtggtggtcctgagcttcgagctgctgcacgcccctgccaccgtgtgcggccccaagaagtccaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggaccctcagaccctggagatcctggacatcaccccttgcagcttcggcggcgtgagcgtgatcacccctggcaccaacaccagcaaccaggtggccgtgctgtaccagggcgtgaactgcaccgaggtgcccgtggccatccacgccgaccagctgacccctacctggagggtgtactccaccggcagcaacgtgttccagaccagggccggctgcctgatcggcgccgagcacgtgaacaacagctacgagtgcgacatccccatcggcgccggcatctgcgccagctaccagacccagaccaacagccccgggagcgccagcagcgtggccagccagagcatcatcgcctacaccatgagcctgggcgtggagaacagcgtggcctacagcaacaacagcatcgccatccccaccaacttcaccatcagcgtgaccaccgagatcctgcccgtgagcatgaccaagaccagcgtggactgcaccatgtatatctgcggcgacagcaccgagtgcagcaacctgctgctccagtacggcagcttctgcacccagctgaacagggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgcctcccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccacccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttccctcagagcgcccctcacggcgtggtgttcctgcacgtgacctacgtgcccgcccaggagaagaacttcaccacagcccctgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccctctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - DNA, 3822 bp, synthetic construct; identical to SEQ ID NO: 10
  source 1..3822: mol_type = other DNA

SEQ ID NO: 31 - protein, 1273 aa, synthetic construct; identical to SEQ ID NO: 14

SEQ ID NO: 32 - DNA, 4238 bp, synthetic construct
  source 1..4238: mol_type = other DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatgttcgtgttcctggtgctgctgcccctggtgtctagccagtgcgtgaacctgaccaccaggacccagctgcctcccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagtccaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtactccagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccctatcaacctggtgagggacctgccccagggcttcagcgccctggagcccctggtggacctgcccatcggcatcaacatcaccaggttccagaccctgctggccctgcacaggagctacctgacccctggcgacagcagctccggctggaccgccggcgccgccgcttactacgtgggctacctgcagcccaggaccttcctgctgaagtacaacgagaacggcaccatcaccgacgccgtggactgcgccctggaccctctgagcgagacaaagtgcaccctgaagtccttcaccgtggagaagggcatctaccagaccagcaacttcagggtgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtataacagcgccagcttcagcaccttcaagtgctacggcgtgagccccaccaagctgaacgacctgtgcttcaccaacgtgtacgccgacagcttcgtgatcaggggcgacgaggtgaggcagatcgcccctggccagaccggcaagatcgccgactacaactacaagctgcccgacgacttcaccggctgcgtgatcgcctggaacagcaacaacctggacagcaaggtgggcggcaactacaactacctgtaccggctgttcagaaagagcaacctgaagcccttcgagagggacatcagcaccgagatctaccaggccggcagcaccccttgcaacggcgtggagggcttcaactgctacttccctctgcagagctacggcttccagcccaccaacggcgtgggctaccagccctacagggtggtggtcctgagcttcgagctgctgcacgcccctgccaccgtgtgcggccccaagaagtccaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggaccctcagaccctggagatcctggacatcaccccttgcagcttcggcggcgtgagcgtgatcacccctggcaccaacaccagcaaccaggtggccgtgctgtaccagggcgtgaactgcaccgaggtgcccgtggccatccacgccgaccagctgacccctacctggagggtgtactccaccggcagcaacgtgttccagaccagggccggctgcctgatcggcgccgagcacgtgaacaacagctacgagtgcgacatccccatcggcgccggcatctgcgccagctaccagacccagaccaacagccccgggagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtatatctgcggcgacagcaccgagtgcagcaacctgctgctccagtacggcagcttctgcacccagctgaacagggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgcctcccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccacccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttccctcagagcgcccctcacggcgtggtgttcctgcacgtgacctacgtgcccgcccaggagaagaacttcaccacagcccctgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccctctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 33 - DNA, 3822 bp, synthetic construct; identical to SEQ ID NO: 11
  source 1..3822: mol_type = other DNA

SEQ ID NO: 34 - protein, 1273 aa, synthetic construct; identical to SEQ ID NO: 15

SEQ ID NO: 35 - DNA, 146 bp, synthetic construct
  source 1..146: mol_type = other DNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccacc

SEQ ID NO: 36 - DNA, 270 bp, synthetic construct
  source 1..270: mol_type = other DNA
actcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 37 - DNA, 170 bp, synthetic construct
  source 1..170: mol_type = other DNA
actcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 38 - DNA, 45 bp, synthetic construct
  source 1..45: mol_type = other DNA
gatgggcggcgcatgagagaagcccagaccaattacctacccaaa

SEQ ID NO: 39 - DNA, 45 bp, synthetic construct
  source 1..45: mol_type = other DNA
gataggcggcgcatgagagaagcccagaccaattacctacccaaa

SEQ ID NO: 40 - DNA, 9739 bp, synthetic construct
  source 1..9739: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggccgccaccatgaaggctatcctggtggtgctgctctacacctttgccacagccaatgctgacaccctgtgtattggctaccatgccaacaacagcacagacacagtggacacagtgttggagaagaatgtgacagtgacccactctgtgaacctgttggaggacaaacacaatggcaaactgtgtaaactgaggggagtggctccactgcacctgggcaagtgtaacattgctggctggattctgggcaaccctgagtgtgagtccctgagcacagcctcctcctggtcctacattgtggagacaccatcctctgacaatggcacttgttaccctggagacttcattgactatgaggaactgagggaacaactttcctctgtgtcctcctttgagaggtttgagatttttccaaagacctcctcctggccaaaccatgacagcaacaagggagtgacagcagcctgtccacatgctggagccaagtccttctacaagaacctgatttggctggtgaagaagggcaactcctacccaaaactgagcaagtcctacatcaatgacaagggcaaggaggtgctggtgctgtggggcatccaccacccaagcacctctgctgaccaacagtccctctaccagaatgctgacgcctatgtgtttgtgggctccagcagatacagcaagaagttcaagcctgagattgccatcagaccaaaggtgagggatcaggagggcaggatgaactactactggaccctggtggaacctggagacaagattacctttgaggctacaggcaacctggtggtgccaagatatgcctttgctatggagaggaatgctggctctggcatcatcatctctgacacacctgtccatgactgtaacaccacttgtcagacaccaaagggagccatcaacacctccctgccattccagaacatccacccaatcaccattggcaagtgtccaaaatatgtcaagagcaccaaactgagactggctacaggactgaggaacatcccaagcatccagagcaggggactgtttggagccattgctggcttcattgagggaggctggacagggatggtggatggctggtatggctaccaccaccagaatgaacagggctctggctatgctgctgacctgaaaagcacccagaatgccattgatgagattaccaacaaggtgaactctgtgattgagaagatgaacacccagttcacagcagtgggcaaggagttcaaccacttggagaagaggattgagaacctgaacaagaaggtggatgatggcttcctggacatctggacctacaatgctgaactgctggtgctgttggagaatgagaggaccctggactaccatgacagcaatgtgaagaacctctatgagaaggtgaggagccaacttaaaaacaatgccaaggagattggcaatggctgttttgagttctaccacaagtgtgacaacacttgtatggagtctgtgaagaatggcacctatgactacccaaaatactctgaggaggctaaactgaacagggaggagattgatggagtgaaattggagagcaccaggatttaccagatcctggccatctacagcaccgtggccagcagcctggtgctggtggtgagcctgggcgccatcagcttctggatgtgcagcaacggcagcttgcagtgcaggatctgcatctaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 41 - DNA, 44 bp, synthetic construct; identical to SEQ ID NO: 5
  source 1..44: mol_type = other DNA

SEQ ID NO: 42 - DNA, 7482 bp, synthetic construct; identical to SEQ ID NO: 6
  source 1..7482: mol_type = other DNA

SEQ ID NO: 43 - DNA, 44 bp, synthetic construct; identical to SEQ ID NO: 7
  source 1..44: mol_type = other DNA

SEQ ID NO: 44 - DNA, 468 bp, synthetic construct; identical to SEQ ID NO: 8
  source 1..468: mol_type = other DNA

SEQ ID NO: 45 - DNA, 368 bp, synthetic construct
  source 1..368: mol_type = other DNA
actcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctag

SEQ ID NO: 46 - DNA, 1701 bp, synthetic construct
  source 1..1701: mol_type = other DNA
atgaaggctatcctggtggtgctgctctacacctttgccacagccaatgctgacaccctgtgtattggctaccatgccaacaacagcacagacacagtggacacagtgttggagaagaatgtgacagtgacccactctgtgaacctgttggaggacaaacacaatggcaaactgtgtaaactgaggggagtggctccactgcacctgggcaagtgtaacattgctggctggattctgggcaaccctgagtgtgagtccctgagcacagcctcctcctggtcctacattgtggagacaccatcctctgacaatggcacttgttaccctggagacttcattgactatgaggaactgagggaacaactttcctctgtgtcctcctttgagaggtttgagatttttccaaagacctcctcctggccaaaccatgacagcaacaagggagtgacagcagcctgtccacatgctggagccaagtccttctacaagaacctgatttggctggtgaagaagggcaactcctacccaaaactgagcaagtcctacatcaatgacaagggcaaggaggtgctggtgctgtggggcatccaccacccaagcacctctgctgaccaacagtccctctaccagaatgctgacgcctatgtgtttgtgggctccagcagatacagcaagaagttcaagcctgagattgccatcagaccaaaggtgagggatcaggagggcaggatgaactactactggaccctggtggaacctggagacaagattacctttgaggctacaggcaacctggtggtgccaagatatgcctttgctatggagaggaatgctggctctggcatcatcatctctgacacacctgtccatgactgtaacaccacttgtcagacaccaaagggagccatcaacacctccctgccattccagaacatccacccaatcaccattggcaagtgtccaaaatatgtcaagagcaccaaactgagactggctacaggactgaggaacatcccaagcatccagagcaggggactgtttggagccattgctggcttcattgagggaggctggacagggatggtggatggctggtatggctaccaccaccagaatgaacagggctctggctatgctgctgacctgaaaagcacccagaatgccattgatgagattaccaacaaggtgaactctgtgattgagaagatgaacacccagttcacagcagtgggcaaggagttcaaccacttggagaagaggattgagaacctgaacaagaaggtggatgatggcttcctggacatctggacctacaatgctgaactgctggtgctgttggagaatgagaggaccctggactaccatgacagcaatgtgaagaacctctatgagaaggtgaggagccaacttaaaaacaatgccaaggagattggcaatggctgttttgagttctaccacaagtgtgacaacacttgtatggagtctgtgaagaatggcacctatgactacccaaaatactctgaggaggctaaactgaacagggaggagattgatggagtgaaattggagagcaccaggatttaccagatcctggccatctacagcaccgtggccagcagcctggtgctggtggtgagcctgggcgccatcagcttctggatgtgcagcaacggcagcttgcagtgcaggatctgcatctaa

SEQ ID NO: 47 - protein, 566 aa, synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 48 - RNA, 2117 nt, synthetic construct
  source 1..2117: mol_type = other RNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccaccatgaaggctatcctggtggtgctgctctacacctttgccacagccaatgctgacaccctgtgtattggctaccatgccaacaacagcacagacacagtggacacagtgttggagaagaatgtgacagtgacccactctgtgaacctgttggaggacaaacacaatggcaaactgtgtaaactgaggggagtggctccactgcacctgggcaagtgtaacattgctggctggattctgggcaaccctgagtgtgagtccctgagcacagcctcctcctggtcctacattgtggagacaccatcctctgacaatggcacttgttaccctggagacttcattgactatgaggaactgagggaacaactttcctctgtgtcctcctttgagaggtttgagatttttccaaagacctcctcctggccaaaccatgacagcaacaagggagtgacagcagcctgtccacatgctggagccaagtccttctacaagaacctgatttggctggtgaagaagggcaactcctacccaaaactgagcaagtcctacatcaatgacaagggcaaggaggtgctggtgctgtggggcatccaccacccaagcacctctgctgaccaacagtccctctaccagaatgctgacgcctatgtgtttgtgggctccagcagatacagcaagaagttcaagcctgagattgccatcagaccaaaggtgagggatcaggagggcaggatgaactactactggaccctggtggaacctggagacaagattacctttgaggctacaggcaacctggtggtgccaagatatgcctttgctatggagaggaatgctggctctggcatcatcatctctgacacacctgtccatgactgtaacaccacttgtcagacaccaaagggagccatcaacacctccctgccattccagaacatccacccaatcaccattggcaagtgtccaaaatatgtcaagagcaccaaactgagactggctacaggactgaggaacatcccaagcatccagagcaggggactgtttggagccattgctggcttcattgagggaggctggacagggatggtggatggctggtatggctaccaccaccagaatgaacagggctctggctatgctgctgacctgaaaagcacccagaatgccattgatgagattaccaacaaggtgaactctgtgattgagaagatgaacacccagttcacagcagtgggcaaggagttcaaccacttggagaagaggattgagaacctgaacaagaaggtggatgatggcttcctggacatctggacctacaatgctgaactgctggtgctgttggagaatgagaggaccctggactaccatgacagcaatgtgaagaacctctatgagaaggtgaggagccaacttaaaaacaatgccaaggagattggcaatggctgttttgagttctaccacaagtgtgacaacacttgtatggagtctgtgaagaatggcacctatgactacccaaaatactctgaggaggctaaactgaacagggaggagattgatggagtgaaattggagagcaccaggatttaccagatcctggccatctacagcaccgtggccagcagcctggtgctggtggtgagcctgggcgccatcagcttctggatgtgcagcaacggcagcttgcagtgcaggatctgcatctaaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 49 - DNA, 146 bp, synthetic construct; identical to SEQ ID NO: 35
  source 1..146: mol_type = other DNA

SEQ ID NO: 50 - DNA, 270 bp, synthetic construct; identical to SEQ ID NO: 36
  source 1..270: mol_type = other DNA

SEQ ID NO: 51 - DNA, 170 bp, synthetic construct; identical to SEQ ID NO: 37
  source 1..170: mol_type = other DNA

SEQ ID NO: 52 - RNA, 1701 nt, synthetic construct
  source 1..1701: mol_type = other RNA
atgaaggctatcctggtggtgctgctctacacctttgccacagccaatgctgacaccctgtgtattggctaccatgccaacaacagcacagacacagtggacacagtgttggagaagaatgtgacagtgacccactctgtgaacctgttggaggacaaacacaatggcaaactgtgtaaactgaggggagtggctccactgcacctgggcaagtgtaacattgctggctggattctgggcaaccctgagtgtgagtccctgagcacagcctcctcctggtcctacattgtggagacaccatcctctgacaatggcacttgttaccctggagacttcattgactatgaggaactgagggaacaactttcctctgtgtcctcctttgagaggtttgagatttttccaaagacctcctcctggccaaaccatgacagcaacaagggagtgacagcagcctgtccacatgctggagccaagtccttctacaagaacctgatttggctggtgaagaagggcaactcctacccaaaactgagcaagtcctacatcaatgacaagggcaaggaggtgctggtgctgtggggcatccaccacccaagcacctctgctgaccaacagtccctctaccagaatgctgacgcctatgtgtttgtgggctccagcagatacagcaagaagttcaagcctgagattgccatcagaccaaaggtgagggatcaggagggcaggatgaactactactggaccctggtggaacctggagacaagattacctttgaggctacaggcaacctggtggtgccaagatatgcctttgctatggagaggaatgctggctctggcatcatcatctctgacacacctgtccatgactgtaacaccacttgtcagacaccaaagggagccatcaacacctccctgccattccagaacatccacccaatcaccattggcaagtgtccaaaatatgtcaagagcaccaaactgagactggctacaggactgaggaacatcccaagcatccagagcaggggactgtttggagccattgctggcttcattgagggaggctggacagggatggtggatggctggtatggctaccaccaccagaatgaacagggctctggctatgctgctgacctgaaaagcacccagaatgccattgatgagattaccaacaaggtgaactctgtgattgagaagatgaacacccagttcacagcagtgggcaaggagttcaaccacttggagaagaggattgagaacctgaacaagaaggtggatgatggcttcctggacatctggacctacaatgctgaactgctggtgctgttggagaatgagaggaccctggactaccatgacagcaatgtgaagaacctctatgagaaggtgaggagccaacttaaaaacaatgccaaggagattggcaatggctgttttgagttctaccacaagtgtgacaacacttgtatggagtctgtgaagaatggcacctatgactacccaaaatactctgaggaggctaaactgaacagggaggagattgatggagtgaaattggagagcaccaggatttaccagatcctggccatctacagcaccgtggccagcagcctggtgctggtggtgagcctgggcgccatcagcttctggatgtgcagcaacggcagcttgcagtgcaggatctgcatctaa

SEQ ID NO: 53 - protein, 566 aa, synthetic construct; identical to SEQ ID NO: 47

SEQ ID NO: 54 - RNA, 22 nt, synthetic construct
  source 1..22: mol_type = other RNA
aaaccgttaccattactgagtt

SEQ ID NO: 55 - RNA, 23 nt, synthetic construct
  source 1..23: mol_type = other RNA
caacggaatcccaaaagcagctg

SEQ ID NO: 56 - RNA, 23 nt, synthetic construct
  source 1..23: mol_type = other RNA
aacattcaacgctgtcggtgagt

SEQ ID NO: 57 - RNA, 22 nt, synthetic construct
  source 1..22: mol_type = other RNA
cacccgtagaaccgaccttgcg

SEQ ID NO: 58 - RNA, 23 nt, synthetic construct
  source 1..23: mol_type = other RNA
taccctgtagatccgaatttgtg

SEQ ID NO: 59 - RNA, 23 nt, synthetic construct
  source 1..23: mol_type = other RNA
taccctgtagaaccgaatttgtg

SEQ ID NO: 60 - RNA, 22 nt, synthetic construct
  source 1..22: mol_type = other RNA
aactggcccacaaagtcccgct

SEQ ID NO: 61 - RNA, 22 nt, synthetic construct
  source 1..22: mol_type = other RNA
aagctgccagttgaagaactgt

SEQ ID NO: 62 - RNA, 21 nt, synthetic construct
  source 1..21: mol_type = other RNA
cattattacttttggtacgcg

SEQ ID NO: 63 - RNA, 21 nt, synthetic construct
  source 1..21: mol_type = other RNA
tattgcacttgtcccggcctg

SEQ ID NO: 64 - RNA, 24 nt, synthetic construct
  source 1..24: mol_type = other RNA
tccctgagaccctttaacctgtga

SEQ ID NO: 65 - RNA, 21 nt, synthetic construct
  source 1..21: mol_type = other RNA
actggacttggagtcagaagg

SEQ ID NO: 66 - RNA, 21 nt, synthetic construct
  source 1..21: mol_type = other RNA
tgagatgaagcactgtagctc

SEQ ID NO: 67 - RNA, 22 nt, synthetic construct
  source 1..22: mol_type = other RNA
tgaggtagtaggttgtatagtt

SEQ ID NO: 68 - RNA, 22 nt, synthetic construct
  source 1..22: mol_type = other RNA
tgaggtagtaggttgtgtggtt

SEQ ID NO: 69 - RNA, 22 nt, synthetic construct
  source 1..22: mol_type = other RNA
tgaggtagtaggttgtatggtt

SEQ ID NO: 70 - RNA, 22 nt, synthetic construct
  source 1..22: mol_type = other RNA
tgaggtagtagattgtatagtt

SEQ ID NO: 71 - RNA, 23 nt, synthetic construct
  source 1..23: mol_type = other RNA
cgcgtaccaaaagtaataatgtg

SEQ ID NO: 72 - RNA, 23 nt, synthetic construct
  source 1..23: mol_type = other RNA
agctcggtctgaggcccctcagt

SEQ ID NO: 73 - RNA, 22 nt, synthetic construct
  source 1..22: mol_type = other RNA
tgtaaacatcctcgactggaag

SEQ ID NO: 74 - RNA, 22 nt, synthetic construct
  source 1..22: mol_type = other RNA
tgtaaacatccccgactggaag

SEQ ID NO: 75 - RNA, 22 nt, synthetic construct
  source 1..22: mol_type = other RNA
tgtaaacatccttgactggaag

SEQ ID NO: 76 - RNA, 22 nt, synthetic construct
  source 1..22: mol_type = other RNA
ttcaagtaatccaggataggct

SEQ ID NO: 77 - RNA, 21 nt, synthetic construct
  source 1..21: mol_type = other RNA
ttcacagtggctaagttctgc

SEQ ID NO: 78 - RNA, 21 nt, synthetic construct
  source 1..21: mol_type = other RNA
ttggtccccttcaaccagctg

SEQ ID NO: 79 - RNA, 22 nt, synthetic construct
  source 1..22: mol_type = other RNA
tttggtccccttcaaccagctg

SEQ ID NO: 80 - RNA, 22 nt, synthetic construct
  source 1..22: mol_type = other RNA
tcctgtactgagctgccccgag

SEQ ID NO: 81 - RNA, 21 nt, synthetic construct
  source 1..21: mol_type = other RNA
cggggcagctcagtacaggat

SEQ ID NO: 82 - RNA, 22 nt, synthetic construct
  source 1..22: mol_type = other RNA
aaaccgttaccattactgagtt

SEQ ID NO: 83 - RNA, 23 nt, synthetic construct
  source 1..23: mol_type = other RNA
agctcggtctgaggcccctcagt

SEQ ID NO: 84 - RNA, 22 nt, synthetic construct
  source 1..22: mol_type = other RNA
actggacttggagtcagaaggc

SEQ ID NO: 85 - RNA, 22 nt, synthetic construct
  source 1..22: mol_type = other RNA
aactggccctcaaagtcccgct

SEQ ID NO: 86 - RNA, 23 nt, synthetic construct
  source 1..23: mol_type = other RNA
caacggaatcccaaaagcagctg

SEQ ID NO: 87 - RNA, 23 nt, synthetic construct
  source 1..23: mol_type = other RNA
aacattcaacgctgtcggtgagt

SEQ ID NO: 88 - RNA, 21 nt, synthetic construct
  source 1..21: mol_type = other RNA
tgagatgaagcactgtagctc

SEQ ID NO: 89 - RNA, 22 nt, synthetic construct
  source 1..22: mol_type = other RNA
tttggtccccttcaaccagctg

SEQ ID NO: 90 - RNA, 21 nt, synthetic construct
  source 1..21: mol_type = other RNA
ttggtccccttcaaccagctg

SEQ ID NO: 91 - RNA, 24 nt, synthetic construct
  source 1..24: mol_type = other RNA
tccctgagaccctttaacctgtga

SEQ ID NO: 92 - RNA, 21 nt, synthetic construct
  source 1..21: mol_type = other RNA
gtacagtactgtgataactga

SEQ ID NO: 93 - RNA, 21 nt, synthetic construct
  source 1..21: mol_type = other RNA
tacagtactgtgataactgaa

SEQ ID NO: 94 - RNA, 22 nt, synthetic construct
  source 1..22: mol_type = other RNA
cacccgtagaaccgaccttgcg

SEQ ID NO: 95 - RNA, 22 nt, synthetic construct
  source 1..22: mol_type = other RNA
tgtaaacatcctcgactggaag

SEQ ID NO: 96 - RNA, 22 nt, synthetic construct
  source 1..22: mol_type = other RNA
tgtaaacatccccgactggaag

SEQ ID NO: 97 - RNA, 22 nt, synthetic construct
  source 1..22: mol_type = other RNA
tgtaaacatccttgactggaag

SEQ ID NO: 98 - RNA, 21 nt, synthetic construct
  source 1..21: mol_type = other RNA
ttcacagtggctaagttctgc

SEQ ID NO: 99 - RNA, 22 nt, synthetic construct
  source 1..22: mol_type = other RNA
ttcaagtaatccaggataggct

SEQ ID NO: 100 - RNA, 22 nt, synthetic construct
  source 1..22: mol_type = other RNA
tattgcacttgtcccggcctgt

SEQ ID NO: 101 - RNA, 22 nt, synthetic construct
  source 1..22: mol_type = other RNA
aagctgccagttgaagaactgt

SEQ ID NO: 102 - RNA, 23 nt, synthetic construct
  source 1..23: mol_type = other RNA
taccctgtagatccgaatttgtg

SEQ ID NO: 103 - RNA, 23 nt, synthetic construct
  source 1..23: mol_type = other RNA
taccctgtagaaccgaatttgtg

SEQ ID NO: 104 - RNA, 22 nt, synthetic construct
  source 1..22: mol_type = other RNA
tgaggtagtaggttgtatagtt

SEQ ID NO: 105 - RNA, 22 nt, synthetic construct
  source 1..22: mol_type = other RNA
tgaggtagtaggttgtgtggtt

SEQ ID NO: 106 - RNA, 22 nt, synthetic construct
  source 1..22: mol_type = other RNA
tgaggtagtaggttgtatggtt

SEQ ID NO: 107 - RNA, 22 nt, synthetic construct
  source 1..22: mol_type = other RNA
tgaggtagtagattgtatagtt

SEQ ID NO: 108 - RNA, 23 nt, synthetic construct
  source 1..23: mol_type = other RNA
caacggaatcccaaaagcagctg

SEQ ID NO: 109 - RNA, 23 nt, synthetic construct
  source 1..23: mol_type = other RNA
aacattcaacgctgtcggtgagt

SEQ ID NO: 110 - RNA, 23 nt, synthetic construct
  source 1..23: mol_type = other RNA
aacattcattgctgtcggtgggt

SEQ ID NO: 111 - RNA, 22 nt, synthetic construct
  source 1..22: mol_type = other RNA
cacccgtagaaccgaccttgcg

SEQ ID NO: 112 - RNA, 23 nt, synthetic construct
  source 1..23: mol_type = other RNA
taccctgtagatccgaatttgtg

SEQ ID NO: 113 - RNA, 22 nt, synthetic construct
  source 1..22: mol_type = other RNA
tagcaccatctgaaatcggtta

SEQ ID NO: 114 - RNA, 22 nt, synthetic construct
  source 1..22: mol_type = other RNA
tagcagcacgtaaatattggcg

SEQ ID NO: 115 - RNA, 22 nt, synthetic construct
  source 1..22: mol_type = other RNA
aagctgccagttgaagaactgt

SEQ ID NO: 116 - RNA, 22 nt, synthetic construct
  source 1..22: mol_type = other RNA
tagcttatcagactgatgttga

SEQ ID NO: 117 - RNA, 21 nt, synthetic construct
  source 1..21: mol_type = other RNA
cataaagtagaaagcactact

SEQ ID NO: 118 - RNA, 22 nt, synthetic construct
  source 1..22: mol_type = other RNA
cattgcacttgtctcggtctga

SEQ ID NO: 119 - RNA, 21 nt, synthetic construct
  source 1..21: mol_type = other RNA
tattgcacttgtcccggcctg

SEQ ID NO: 120 - RNA, 22 nt, synthetic construct
  source 1..22: mol_type = other RNA
tcagtgcactacagaactttgt

SEQ ID NO: 121 - RNA, 21 nt, synthetic construct
  source 1..21: mol_type = other RNA
actggacttggagtcagaagg

SEQ ID NO: 122 - RNA, 22 nt, synthetic construct
  source 1..22: mol_type = other RNA
tgagaactgaattccataggct

SEQ ID NO: 123 - RNA, 22 nt, synthetic construct
  source 1..22: mol_type = other RNA
agagcttagctgattggtgaac

SEQ ID NO: 124 - RNA, 22 nt, synthetic construct
  source 1..22: mol_type = other RNA
tgaggtagtaggttgtatagtt

SEQ ID NO: 125 - RNA, 22 nt, synthetic construct
  source 1..22: mol_type = other RNA
tgaggtagtagattgtatagtt

SEQ ID NO: 126 - RNA, 22 nt, synthetic construct
  source 1..22: mol_type = other RNA
tgaggtagtagtttgtacagtt

SEQ ID NO: 127 - RNA, 22 nt, synthetic construct
  source 1..22: mol_type = other RNA
tgaggtagtagtttgtgctgtt

SEQ ID NO: 128 - RNA, 23 nt, synthetic construct
  source 1..23: mol_type = other RNA
agcagcattgtacagggctatga

SEQ ID NO: 129 - RNA, 23 nt, synthetic construct
  source 1..23: mol_type = other RNA
agctacattgtctgctgggtttc

SEQ ID NO: 130 - RNA, 21 nt, synthetic construct
  source 1..21: mol_type = other RNA
agctacatctggctactgggt

SEQ ID NO: 131 - RNA, 22 nt, synthetic construct
  source 1..22: mol_type = other RNA
tggctcagttcagcaggaacag

SEQ ID NO: 132 - RNA, 22 nt, synthetic construct
  source 1..22: mol_type = other RNA
agggcttagctgcttgtgagca

SEQ ID NO: 133 - RNA, 22 nt, synthetic construct
  source 1..22: mol_type = other RNA
tgtaaacatccccgactggaag

SEQ ID NO: 134 - RNA, 22 nt, synthetic construct
  source 1..22: mol_type = other RNA
tgtcagtttgtcaaatacccca

SEQ ID NO: 135 - RNA, 23 nt, synthetic construct
  source 1..23: mol_type = other RNA
cgtgtatttgacaagctgagttg

SEQ ID NO: 136 - RNA, 23 nt, synthetic construct
  source 1..23: mol_type = other RNA
ttaatgctaattgtgataggggt

SEQ ID NO: 137 - RNA, 22 nt, synthetic construct
  source 1..22: mol_type = other RNA
ttcaagtaatccaggataggct

SEQ ID NO: 138 - RNA, 21 nt, synthetic construct
  source 1..21: mol_type = other RNA
ttcaagtaattcaggataggt

SEQ ID NO: 139 - RNA, 21 nt, synthetic construct
  source 1..21: mol_type = other RNA
ttcacagtggctaagttccgc

SEQ ID NO: 140 - RNA, 21 nt, synthetic construct
  source 1..21: mol_type = other RNA
ttcacagtggctaagttctgc

SEQ ID NO: 141 - RNA, 23 nt, synthetic construct
  source 1..23: mol_type = other RNA
tgaggggcagagagcgagacttt

SEQ ID NO: 142 - RNA, 23 nt, synthetic construct
  source 1..23: mol_type = other RNA
agctcggtctgaggcccctcagt

SEQ ID NO: 143 - RNA, 22 nt, synthetic construct
  source 1..22: mol_type = other RNA
actggacttggagtcagaaggc

SEQ ID NO: 144 - RNA, 23 nt, synthetic construct
  source 1..23: mol_type = other RNA
tctcacacagaaatcgcacccgt

SEQ ID NO: 145 - RNA, 22 nt, synthetic construct
  source 1..22: mol_type = other RNA
cgtgtatttgacaagctgagtt

SEQ ID NO: 146 - RNA, 22 nt, synthetic construct
  source 1..22: mol_type = other RNA
tgtcagtttgtcaaatacccca

SEQ ID NO: 147 - RNA, 23 nt, synthetic construct
  source 1..23: mol_type = other RNA
caacggaatcccaaaagcagctg

SEQ ID NO: 148 - RNA, 22 nt, synthetic construct
  source 1..22: mol_type = other RNA
caaagaattctccttttgggct

SEQ ID NO: 149 - RNA, 23 nt, synthetic construct
  source 1..23: mol_type = other RNA
aacattcaacgctgtcggtgagt

SEQ ID NO: 150 - RNA, 22 nt, synthetic construct
  source 1..22: mol_type = other RNA
tgagaactgaattccataggct

SEQ ID NO: 151 - RNA, 21 nt, synthetic construct
  source 1..21: mol_type = other RNA
cataaagtagaaagcactact

SEQ ID NO: 152 - RNA, 22 nt, synthetic construct
  source 1..22: mol_type = other RNA
gtagtgtttcctactttatgga

SEQ ID NO: 153 - RNA, 23 nt, synthetic construct
  source 1..23: mol_type = other RNA
tgtagtgtttcctactttatgga

SEQ ID NO: 154 - RNA, 21 nt, synthetic construct
  source 1..21: mol_type = other RNA
taccacagggtagaaccacgg

SEQ ID NO: 155 - RNA, 22 nt, synthetic construct
  source 1..22: mol_type = other RNA
accacagggtagaaccacggac

SEQ ID NO: 156 - RNA, 23 nt, synthetic construct
  source 1..23: mol_type = other RNA
agcagcattgtacagggctatga

SEQ ID NO: 157 - RNA, 23 nt, synthetic construct
  source 1..23: mol_type = other RNA
agcagcattgtacagggctatca

SEQ ID NO: 158 - RNA, 22 nt, synthetic construct
  source 1..22: mol_type = other RNA
tgtaaacatcctcgactggaag

SEQ ID NO: 159 - RNA, 23 nt, synthetic construct
  source 1..23: mol_type = other RNA
tgtaaacatcctacactctcagc

SEQ ID NO: 160 - RNA, 22 nt, synthetic construct
  source 1..22: mol_type = other RNA
tgtaaacatccccgactggaag

SEQ ID NO: 161 - RNA, 22 nt, synthetic construct
  source 1..22: mol_type = other RNA
tgtaaacatccttgactggaag

SEQ ID NO: 162 - RNA, 22 nt, synthetic construct
  source 1..22: mol_type = other RNA
cactagattgtgagctcctgga

SEQ ID NO: 163 - RNA, 22 nt, synthetic construct
  source 1..22: mol_type = other RNA
agagcttagctgattggtgaac

SEQ ID NO: 164 - RNA, 22 nt, synthetic construct
  source 1..22: mol_type = other RNA
agggcttagctgcttgtgagca

SEQ ID NO: 165 - RNA, 21 nt, synthetic construct
  source 1..21: mol_type = other RNA
ttcacagtggctaagttccgc

SEQ ID NO: 166 - RNA, 21 nt, synthetic construct
  source 1..21: mol_type = other RNA
ttcacagtggctaagttctgc

SEQ ID NO: 167 - RNA, 22 nt, synthetic construct
  source 1..22: mol_type = other RNA
ttcaagtaatccaggataggct

SEQ ID NO: 168 - RNA, 21 nt, synthetic construct
  source 1..21: mol_type = other RNA
ttcaagtaattcaggataggt

SEQ ID NO: 169 - RNA, 22 nt, synthetic construct
  source 1..22: mol_type = other RNA
cattgcacttgtctcggtctga

SEQ ID NO: 170 - RNA, 22 nt, synthetic construct
  source 1..22: mol_type = other RNA
tattgcacttgtcccggcctgt

SEQ ID NO: 171 - RNA, 22 nt, synthetic construct
  source 1..22: mol_type = other RNA
tggctcagttcagcaggaacag

SEQ ID NO: 172 - RNA, 22 nt, synthetic construct
  source 1..22: mol_type = other RNA
aagctgccagttgaagaactgt

SEQ ID NO: 173 - RNA, 22 nt, synthetic construct
  source 1..22: mol_type = other RNA
tagcttatcagactgatgttga

SEQ ID NO: 174 - RNA, 21 nt, synthetic construct
  source 1..21: mol_type = other RNA
caacaccagtcgatgggctgt

SEQ ID NO: 175 - RNA, 22 nt, synthetic construct
  source 1..22: mol_type = other RNA
tagcagcacgtaaatattggcg

SEQ ID NO: 176 - RNA, 22 nt, synthetic construct
  source 1..22: mol_type = other RNA
tgaggtagtaggttgtatagtt

SEQ ID NO: 177 - RNA, 22 nt, synthetic construct
  source 1..22: mol_type = other RNA
tgaggtagtaggttgtgtggtt

SEQ ID NO: 178 - RNA, 22 nt, synthetic construct
  source 1..22: mol_type = other RNA
tgaggtagtaggttgtatggtt

SEQ ID NO: 179 - RNA, 22 nt, synthetic construct
  source 1..22: mol_type = other RNA
agaggtagtaggttgcatagtt

SEQ ID NO: 180 - RNA, 22 nt, synthetic construct
  source 1..22: mol_type = other RNA
tgaggtaggaggttgtatagtt

SEQ ID NO: 181 - RNA, 22 nt, synthetic construct
  source 1..22: mol_type = other RNA
tgaggtagtagattgtatagtt

SEQ ID NO: 182 - RNA, 22 nt, synthetic construct
  source 1..22: mol_type = other RNA
tgaggtagtagtttgtacagtt

SEQ ID NO: 183 - RNA, 22 nt, synthetic construct
  source 1..22: mol_type = other RNA
tgaggtagtagtttgtgctgtt

SEQ ID NO: 184 - RNA, 22 nt, synthetic construct
  source 1..22: mol_type = other RNA
tgaggtagtaagttgtattgtt

SEQ ID NO: 185 - DNA, 6993 bp, synthetic construct
  source 1..6993: mol_type = other DNA
gccgtggacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctgagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctgctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtgctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccccacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgaccgagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtacaggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacccctgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaacagggtgatcaccagggaggagctggaggccctgacccctagcaggacccctagcaggagcgtgagcaggaccagcctggtgagcaaccctcccggcgtgaacagggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtgctggagaggaccgagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctgcaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaacagggtgggcatcctgagcgagctgtgcaaggccgtggagagcaggtacgagacagtgggcaccagcatcatcgtgatggccatgaccaccctggccagcagcgtgaagtccttcagctacctgagg

SEQ ID NO: 186 - DNA, 9690 bp, synthetic construct
  source 1..9690: mol_type = other DNA
atgggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtggacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggacaggcagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctgagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctgctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtgctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccccacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgaccgagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtacaggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacccctgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaacagggtgatcaccagggaggagctggaggccctgacccctagcaggacccctagcaggagcgtgagcaggaccagcctggtgagcaaccctcccggcgtgaacagggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtgctggagaggaccgagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctgcaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtgctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaacagggtgggcatcctgagcgagctgtgcaaggccgtggagagcaggtacgagacagtgggcaccagcatcatcgtgatggccatgaccaccctggccagcagcgtgaagtccttcagctacctgaggggggcccctataactctctacggctaacctgaatggactacgacatagtctagtccgccaaggccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgacagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagtccctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 187 - protein, 2493 aa, synthetic construct
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGAGSVETPRGLIKVTSYDGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARLFKFSRVCKPKSSLEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQRRFDAGAYIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 188 - protein, 2493 aa, synthetic construct; identical to SEQ ID NO: 187

SEQ ID NO: 189 - RNA, 129 nt, synthetic construct
  source 1..129: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatag

SEQ ID NO: 190 - RNA, 28 nt, synthetic construct
  source 1..28: mol_type = other RNA
attattacatcaaaacaaaaagccgcca

SEQ ID NO: 191 - RNA, 245 nt, synthetic construct
  source 1..245: mol_type = other RNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctac

SEQ ID NO: 192 - RNA, 177 nt, synthetic construct
  source 1..177: mol_type = other RNA
tgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagtgttgggttgcg

SEQ ID NO: 193 - RNA, 89 nt, synthetic construct
  source 1..89: mol_type = other RNA
aattattggttaaagaagtatattagtgctaatttccctccgtttgtcctagcttttctcttctgtcaaccccacacgcctttggcaca

SEQ ID NO: 194 - RNA, 569 nt, synthetic construct
  source 1..569: mol_type = other RNA
ctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataat

SEQ ID NO: 195 - RNA, 21 nt, synthetic construct
  source 1..21: mol_type = other RNA
cacaaagagtaaagaagaaca

SEQ ID NO: 196 - RNA, 21 nt, synthetic construct
  source 1..21: mol_type = other RNA
aacactaaaagtagaagaaaa

SEQ ID NO: 197 - RNA, 21 nt, synthetic construct
  source 1..21: mol_type = other RNA
ctcagaaagataagatcagcc

SEQ ID NO: 198 - RNA, 21 nt, synthetic construct
  source 1..21: mol_type = other RNA
aaccaatcgaaagaaaccaaa

SEQ ID NO: 199 - RNA, 21 nt, synthetic construct
  source 1..21: mol_type = other RNA
ctctaatcaccaggagtaaaa

SEQ ID NO: 200 - RNA, 21 nt, synthetic construct
  source 1..21: mol_type = other RNA
gagagagatcttaacaaaaaa

SEQ ID NO: 201 - RNA, 21 nt, synthetic construct
  source 1..21: mol_type = other RNA
tgtgtaacaacaacaacaaca

SEQ ID NO: 202 - RNA, 21 nt, synthetic construct
  source 1..21: mol_type = other RNA
ccgcagtaggaagagaaagcc

SEQ ID NO: 203 - RNA, 21 nt, synthetic construct
  source 1..21: mol_type = other RNA
aaaaaaaaaagaaatcataaa

SEQ ID NO: 204 - RNA, 21 nt, synthetic construct
  source 1..21: mol_type = other RNA
gagagaagaaagaagaagacg

SEQ ID NO: 205 - RNA, 21 nt, synthetic construct
  source 1..21: mol_type = other RNA
caattaaaaatacttaccaaa

SEQ ID NO: 206 - RNA, 21 nt, synthetic construct
  source 1..21: mol_type = other RNA
gcaaacagagtaagcgaaacg

SEQ ID NO: 207 - RNA, 21 nt, synthetic construct
  source 1..21: mol_type = other RNA
gcgaagaagacgaacgcaaag

SEQ ID NO: 208 - RNA, 21 nt, synthetic construct
  source 1..21: mol_type = other RNA
ttaggactgtattgactggcc

SEQ ID NO: 209 - RNA, 21 nt, synthetic construct
  source 1..21: mol_type = other RNA
atcatcggaattcggaaaaag

SEQ ID NO: 210 - RNA, 21 nt, synthetic construct
  source 1..21: mol_type = other RNA
aaaacaaaagttaaagcagac

SEQ ID NO: 211 - RNA, 21 nt, synthetic construct
  source 1..21: mol_type = other RNA
tttatctcaaataagaaggca

SEQ ID NO: 212 - RNA, 21 nt, synthetic construct
  source 1..21: mol_type = other RNA
ggtggggaggtgagatttctt

SEQ ID NO: 213 - RNA, 21 nt, synthetic construct
  source 1..21: mol_type = other RNA
tgattaggaaactacaaagcc

SEQ ID NO: 214 - RNA, 21 nt, synthetic construct
  source 1..21: mol_type = other RNA
catttttcaatttcataaaac

SEQ ID NO: 215 - RNA, 21 nt, synthetic construct
  source 1..21: mol_type = other RNA
ttacttttaagcccaacaaaa

SEQ ID NO: 216 - RNA, 21 nt, synthetic construct
  source 1..21: mol_type = other RNA
ggcgtgtgtgtgtgttgttga

SEQ ID NO: 217 - RNA, 21 nt, synthetic construct
  source 1..21: mol_type = other RNA
gtggtgaaggggaaggtttag

SEQ ID NO: 218 - RNA, 21 nt, synthetic construct
  source 1..21: mol_type = other RNA
ttgtttttttttggtttggtt

SEQ ID NO: 219 - RNA, 158 nt, synthetic construct
  source 1..158: mol_type = other RNA
ctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacat

SEQ ID NO: 220 - RNA, 166 nt, synthetic construct
  source 1..166: mol_type = other RNA
tgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccat

SEQ ID NO: 221 - RNA, 143 nt, synthetic construct
  source 1..143: mol_type = other RNA
acgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 222 - RNA, 220 nt, synthetic construct
  source 1..220: mol_type = other RNA
tagagcggcaaaccctagctacactccatagctagtttcttttttttttgtttttttttttttttttttttttttttttttttttttttcctttcttttccttctttttttcctcttttcttggtggctccatcttagccctagtcacggctagctgtgaaaggtccgtgagccgcatgactgcagagagtgccgtaactggtctctctgcagatcatgt

SEQ ID NO: 223 - RNA, 170 nt, synthetic construct
  source 1..170: mol_type = other RNA
acacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctac

SEQ ID NO: 224 - RNA, 110 nt, synthetic construct
  source 1..110: mol_type = other RNA
gctggagcctcggtagccgttcctcctgcccgctgggcctcccaacgggccctcctcccctccttgcaccggcccttcctggtctttgaataaagtctgagtgggcagca

SEQ ID NO: 225 - RNA, 123 nt, synthetic construct
  source 1..123: mol_type = other RNA
tagtgcagtcactggcacaacgcgttgcccggtaagccaatcgggtatacacggtcgtcatactgcagacagggttcttctactttgcaagatagtctagagtagtaaaataaatagtataag

SEQ ID NO: 226 - RNA, 44 nt, synthetic construct
  source 1..44: mol_type = other RNA
gtcagctttcaaactctttgtttcttgtttgttgattgagaata

SEQ ID NO: 227 - RNA, 54 nt, synthetic construct
  source 1..54: mol_type = other RNA
ctctcgcctgagaaaaaaaatccacgaaccaatttctcagcaaccagcagcacg

SEQ ID NO: 228 - RNA, 52 nt, synthetic construct
  source 1..52: mol_type = other RNA
acctgtgagggttcgaaggaagtagcagtgttttttgttcctagaggaagag

SEQ ID NO: 229 - RNA, 71 nt, synthetic construct
  source 1..71: mol_type = other RNA
acacagaaacattcgcaaaaacaaaatcccagtatcaaaattcttctctttttttcatatttcgcaaagac

SEQ ID NO: 230 - RNA, 52 nt, synthetic construct
  source 1..52: mol_type = other RNA
cagaaaaatttgctacattgtttcacaaacttcaaatattattcatttattt

SEQ ID NO: 231-232 - skipped (2 entries)

SEQ ID NO: 233 - RNA, 21 nt, synthetic construct
  source 1..21: mol_type = other RNA
aacttaaaaaaaaaaatcaaa

SEQ ID NO: 234 - RNA, 92 nt, synthetic construct
  source 1..92: mol_type = other RNA
tcaagcttttggaccctcgtacagaagctaatacgactcactatagggaaataagagagaaaagaagagtaagaagaaatataagagccacc

SEQ ID NO: 235 - RNA, 53 nt, synthetic construct
  source 1..53: mol_type = other RNA
cacatttgcttctgacatagttgtgttgactcacaaccccagaaacagacatc

SEQ ID NO: 236 - RNA, 50 nt, synthetic construct
  source 1..50: mol_type = other RNA
acatttgcttctgacacaactgtgttcactagcaacctcaaacagacacc

SEQ ID NO: 237 - RNA, 46 nt, synthetic construct
  source 1..46: mol_type = other RNA
tgcacacagatcacctttcctatcaaccccactagcctctggcaaa

SEQ ID NO: 238 - RNA, 66 nt, synthetic construct
  source 1..66: mol_type = other RNA
cataaaccctggcgcgctcgcgggccggcactcttctggtccccacagactcagagagaacccacc

SEQ ID NO: 239 - RNA, 58 nt, synthetic construct
  source 1..58: mol_type = other RNA
ataaaaagaccagcagatgccccacagcactgctcttccagaggcaagaccaaccaag

SEQ ID NO: 240 - RNA, 177 nt, synthetic construct
  source 1..177: mol_type = other RNA
agacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcacagaagtccactcattcttggcagg

SEQ ID NO: 241 - RNA, 93 nt, synthetic construct
  source 1..93: mol_type = other RNA
agataaaaagccagctccagcaggcgctgctcactcctccccatcctctccctctgtccctctgtccctctgaccctgcactgtcccagcacc

SEQ ID NO: 242 - RNA, 30 nt, synthetic construct
  source 1..30: mol_type = other RNA
tatatccgtggtttcctgctacctccaacc

SEQ ID NO: 243 - RNA, 34 nt, synthetic construct
  source 1..34: mol_type = other RNA
ggcaccaccactgacctgggacagtgaatcgaca

SEQ ID NO: 244 - RNA, 79 nt, synthetic construct
  source 1..79: mol_type = other RNA
attcatgaaaatccactactccagacagacggctttggaatccaccagctacatccagctccctgaggcagagttgaga

SEQ ID NO: 245 - RNA, 116 nt, synthetic construct
  source 1..116: mol_type = other RNA
aatattagagtctcaacccccaataaatataggactggagatgtctgaggctcattctgccctcgagcccaccgggaacgaaagagaagctctatctcccctccaggagcccagct

SEQ ID NO: 246 - RNA, 79 nt, synthetic construct
  source 1..79: mol_type = other RNA
aggatgggaactaggagtggcagcaatcctttctttcagctggagtgctcctcaggagccagccccacccttagaaaag

SEQ ID NO: 247 - RNA, 111 nt, synthetic construct
  source 1..111: mol_type = other RNA
agggggagccctataattggacaagtctgggatccttgagtcctactcagccccagcggaggtgaaggacgtccttccccaggagccgactggccaatcacaggcaggaag

SEQ ID NO: 248 - RNA, 223 nt, synthetic construct
  source 1..223: mol_type = other RNA
agacgggtggggcggggcccaactgtccccagctccttcagccctttctgtccctcccagtgaggccagctgcggtgaagagggtgctctcttgcctggagttccctctgctacggctgccccctcccagccctggcccactaagccagacccagctgtcgccattcccacttctggtcctgccacctcctgagctgccttcccgcctggtctgggtagagtc

SEQ ID NO: 249 - RNA, 137 nt, synthetic construct
  source 1..137: mol_type = other RNA
cagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 250 - RNA, 200 nt, synthetic construct
  source 1..200: mol_type = other RNA
gggagaaagcttaccatggtgccccaggccctgctcttggtcccgctgctggtttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctac

SEQ ID NO: 251 - RNA, 239 nt, synthetic construct
  source 1..239: mol_type = other RNA
ggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctac

SEQ ID NO: 252 - RNA, 147 nt, synthetic construct
  source 1..147: mol_type = other RNA
gaataaatgtatagggggaaaggcaggagccttggggtcgaggaaaacaggtagggtataaaaagggcacgcaagggaccaagtccagcatcctagagtccagattccaaactgctcagagtcctgtggacagatcactgcttggca

SEQ ID NO: 253 - RNA, 35 nt, synthetic construct
  source 1..35: mol_type = other RNA
gacacttctgattctgacagactcaggaagaaacc

SEQ ID NO: 254 - RNA, 158 nt, synthetic construct
  source 1..158: mol_type = other RNA
tgcaaacacagaaatggaggaggaggggaaggaggaggaggaggagaaggaggaggaggtggtggtggtggtggtgggataaaacccctgaggcataaagggctcggccggagtcagcacagcccagcccttccagagagaggcaagagaggtccacg

SEQ ID NO: 255 - RNA, 189 nt, synthetic construct
  source 1..189: mol_type = other RNA
ctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgacagg

SEQ ID NO: 256 - RNA, 212 nt, synthetic construct
  source 1..212: mol_type = other RNA
ataggtaattttagaaatagatctgatttgtatctgagacattttagtgaagtggtgagatataagacataatcagaagacatatctacctgaagactttaaggggagagctccctcccccacctggcctctggacctctcagatttaggggaaagaaccagttttcggagtgatcgtctcagtcagcaccatctctgtaggagcatcggcc

SEQ ID NO: 257 - RNA, 101 nt, synthetic construct
  source 1..101: mol_type = other RNA
agagaggagagccatataaagagccagcggctacagccccagctcgcctctgcccacccctgccccttaccccttcattccttccacctttttccttcact

SEQ ID NO: 258 - RNA, 43 nt, synthetic construct
  source 1..43: mol_type = other RNA
tttaaaaggaaagtggttacagggaggccatgcccatgggttt

SEQ ID NO: 259 - RNA, 34 nt, synthetic construct
  source 1..34: mol_type = other RNA
agtccttagactgcacagcagaacagaaggcatg

SEQ ID NO: 260 - RNA, 102 nt, synthetic construct
  source 1..102: mol_type = other RNA
cccccatatcccccttggctcccattgcttaaatacagactaggacagggctctgtctcctcagcctcggtcaccacccagctctgggacagcaagctgaaa

SEQ ID NO: 261 - RNA, 62 nt, synthetic construct
  source 1..62: mol_type = other RNA
agtcagtcctccttcgcttcagctccagttctcctcatgagccatccctaaacgcagacacc

SEQ ID NO: 262 - RNA, 201 nt, synthetic construct
  source 1..201: mol_type = other RNA
tttcctctgccctgctgtgaagggggagagaacaacccgcctcgtgacagggggctggcacagcccgccctagccctgaggagggggcgggacagggggagtcctataattggaccggtctgggatccgatcccctgctcagaccctggaggctaaggacttgtttcggaaggagctgactggccaatcacaattgcgaag

SEQ ID NO: 263 - RNA, 95 nt, synthetic construct
  source 1..95: mol_type = other RNA
ggccggccaccgggtttgggagcagcccaggctcaccttaaccggagcggtgcggacggtcccgcggcgacagggctaatctcggcaggttcgcg

SEQ ID NO: 264 - RNA, 59 nt, synthetic construct
  source 1..59: mol_type = other RNA
gtcctggactgactcccacaactctgccagtctccagcccctgcccttcagtggtacag

SEQ ID NO: 265 - RNA, 59 nt, synthetic construct
  source 1..59: mol_type = other RNA
tttaagtcaacaccaggaactaggacacagttgtccaggtgctgttggccagtcccaac

SEQ ID NO: 266 - RNA, 81 nt, synthetic construct
  source 1..81: mol_type = other RNA
aaggagctggggagtggagtgtaggcactataacctgaaagacgtggtcctgacaggaggacaattctattccctaccaaa

SEQ ID NO: 267 - RNA, 25 nt, synthetic construct
  source 1..25: mol_type = other RNA
accagccagaagccacagtctcatc

SEQ ID NO: 268 - RNA, 218 nt, synthetic construct
  source 1..218: mol_type = other RNA
aaacagagcaggcaggggccctgattcactggccgctggggccagggttgggggctgggggtgcccacagagcttgactagtgggatttgggggggcagtgggtgcagcgagcccggtccgttgactgccagcctgccggcaggtagacaccggccgtgggtgggggaggcggctagctcagtggccttgggccgcgtggcctggtggcagcggagcc

SEQ ID NO: 269 - RNA, 67 nt, synthetic construct
  source 1..67: mol_type = other RNA
ggacttcagcaggactgctcgaaacatcccacttccagcactgcctgcggtgaaggaaccagcagcc

SEQ ID NO: 270 - RNA, 252 nt, synthetic construct
  source 1..252: mol_type = other RNA
agggcctcgtggggggcgggaaggtactgtcccatataagcctctgctcttggggctcaaccgctcgcacccgctgcgctgcacagggggagaaaaggagcccagggtgtgagccggacaacttctggtcctctccttccatctccttaccggcgtccccacctcaggacttttcccgcaggctgcgaggggacccacagttcgtggccacttgcctcctggggagggcgactctcctcccatccactcaag

SEQ ID NO: 271 - RNA, 253 nt, synthetic construct
  source 1..253: mol_type = other RNA
gggggaaaaaaaaacagccaaaatatgccaaaaagcttctcacaacagctcctcagtagaagcaggggccacttgggaaagccagggcctggacgctaatgttccaggctacatcataggtcccttttcgctcagtgaggccaccatcaccacaccatggccacgtaggcctccagccagggcaacaggacctggaggccacccaagactgcagctggctgccgctgggtccccgggccagctcttggccccg

SEQ ID NO: 272 - RNA, 136 nt, synthetic construct
  source 1..136: mol_type = other RNA
gaaccgcggcgaggaggggggtcggaggcccagacttataaaggctgctggacccgcgctacccgccagaccccgccgcccggatcccccgcgctgcctgtcgccccacgtgaccacactactaagcttggtcgcc

SEQ ID NO: 273 - RNA, 120 nt, synthetic construct
  source 1..120: mol_type = other RNA
agggactcatcaaccaggcctggcctctgagttcaacgcagagctagctgggaaatgttccggatgttggccaaggccagtgtgacgctgggctccagagcggcaggttgggtccggacc

SEQ ID NO: 274 - RNA, 146 nt, synthetic construct
  source 1..146: mol_type = other RNA
gctgcccctgtgctgactgctgacagctgactgacgctcgcagctagcaggtacttctgggttgctagcccagagccctgggccggtgaccctgttttccctacttcccgtctttgaccttgggtgccttccaaccttctgttgcc

SEQ ID NO: 275 - RNA, 219 nt, synthetic construct
  source 1..219: mol_type = other RNA
gggtgctaaaagaatcactagggtggggaggcggtcccagtggggcgggtaggggtgtgtgccaggtggtaccgggtattggctggaggaagggcagcccggggttcggggcggtccctgaatctaaaggccctcggctagtctgatccttgccctaagcatagtcccgttagccaaccccctacccgccgtgggctctgctgcccggtgctcgtcagc

SEQ ID NO: 276 - RNA, 239 nt, synthetic construct
  source 1..239: mol_type = other RNA
aggaggaccttggccagcgggcagaatggcagttggtagaggaagggagcaagggggtgtttcctgggacaggggggcggagacctggagactataggctcccccaggactcaagttcattgagtttctgcagacactgaacggctttcagtcttcccgctgtgactatcacctgtgggctccacctgcctgcacctttagtcagcacctttagccagcacctgcgccagaccccagca

SEQ ID NO: 277 - RNA, 14 nt, synthetic construct
  source 1..14: mol_type = other RNA
aggcgccggtcagg

SEQ ID NO: 278 - RNA, 10 nt, synthetic construct
  source 1..10: mol_type = other RNA
accatcaacc

SEQ ID NO: 279 - RNA, 119 nt, synthetic construct
  source 1..119: mol_type = other RNA
tctgccccaccctgtcctctggaacctctgcgagatttagaggaaagaaccagttttcaggcggattgcctcagatcacactatctccacttgcccagccctgtggaagattagcggcc

SEQ ID NO: 280 - RNA, 142 nt, synthetic construct
  source 1..142: mol_type = other RNA
accccctttcctgctcttgcctgtgaacaatggttaattgttcccaagagagcatctgtcagttgttggcaaaatgataaagacatttgaaaatctgtcttctgacaaataaaaagcatttatttcactgcaatgatgtttt

SEQ ID NO: 281 - RNA, 134 nt, synthetic construct
  source 1..134: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 282 - RNA, 106 nt, synthetic construct
  source 1..106: mol_type = other RNA
tggcatccctgtgacccctccccagtgcctctcctggccctggaagttgccactccagtgcccaccagccttgtcctaataaaattaagttgcatcattttgtctg

SEQ ID NO: 283 - RNA, 80 nt, synthetic construct
  source 1..80: mol_type = other RNA
aatgttcttattctttgcacctcttcctatttttggtttgtgaacagaagtaaaaataaatacaaactacttccatctca

SEQ ID NO: 284 - RNA, 178 nt, synthetic construct
  source 1..178: mol_type = other RNA
ccacacccccattcccccactccagataaagcttcagttatatctcacgtgtctggagttctttgccaagagggagaggctgaaatccccagccgcctcacctgcagctcagctccatcctacttgaaacctcacctgttcccaccgcattttctcctggcgttcgcctgctagtgtg

SEQ ID NO: 285 - RNA, 101 nt, synthetic construct
  source 1..101: mol_type = other RNA
aacctacctgccctgcccccgtcccctcccttccttatttattcctgctgccccagaacataggtcttggaataaaatggctggttcttttgttttccaaa

SEQ ID NO: 286 - RNA, 234 nt, synthetic construct
  source 1..234: mol_type = other RNA
actaagttaaatatttctgcacagtgttcccatggccccttgcatttccttcttaactctctgttacacgtcattgaaactacacttttttggtctgtttttgtgctagactgtaagttccttgggggcagggcctttgtctgtctcatctctgtattcccaaatgcctaacagtacagagccatgactcaataaatacatgttaaatggatgaatgaattcctctgaaactct

SEQ ID NO: 287 - RNA, 171 nt, synthetic construct
  source 1..171: mol_type = other RNA
gcaccccagctggggccaggctgggtcgccctggactgtgtgctcaggagccctgggaggctctggagcccactgtacttgctcttgatgcctggcggggtggggtggggggggtgctgggcccctgcctctctgcaggtccctaataaagctgtgtggcagtctgactcc

SEQ ID NO: 288 - RNA, 110 nt, synthetic construct
  source 1..110: mol_type = other RNA
gattcgtcagtagggttgtaaaggtttttcttttcctgagaaaacaaccttttgttttctcaggttttgctttttggcctttccctagctttaaaaaaaaaaaagcaaaa

SEQ ID NO: 289 - RNA, 74 nt, synthetic construct
  source 1..74: mol_type = other RNA
ggactagttataagactgactagcccgatgggcctcccaacgggccctcctcccctccttgcaccgagattaat

SEQ ID NO: 290 - RNA, 201 nt, synthetic construct
  source 1..201: mol_type = other RNA
ggactagtgcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctagatct

SEQ ID NO: 291 - RNA, 105 nt, synthetic construct
  source 1..105: mol_type = other RNA
ccactcaccagtgtctctgctgcactctcctgtgcctccctgccccctggcaactgccacccctgcgctttgtcctaataaaattaagatgcatcatatcacccg

SEQ ID NO: 292 - RNA, 105 nt, synthetic construct
  source 1..105: mol_type = other RNA
gctgccttctgcggggcttgccttctggccatgcccttcttctctcccttgcacctgtacctcttggtctttgaataaagcctgagtaggaagaaaaaaaaaaaa

SEQ ID NO: 293 - RNA, 163 nt, synthetic construct
  source 1..163: mol_type = other RNA
ttcagggctcactagaaggctgcacatggcagggcaggctgggagccatggaagagggggaagtggaagggttgggctatactctgatgggttctagccctgcactgctcagtcaacaataaaaaaatgtgctttggacccataaaaaaaaaaaaaaaaaaaa

SEQ ID NO: 294 - RNA, 604 nt, synthetic construct
  source 1..604: mol_type = other RNA
gagactcagcccaggaggaccaggatcttgccaaagcagtagcatcccatttgtaccaaaacagtgttcttgctctataaaccgtgttagcagctcaggaagatgccgtgaagcattcttattaaaccacctgctatttcattcaaactgtgtttcttttttatttcctcatttttctcccctgctcctaaaacccaaaatcttctaaagaattctagaaggtatgcgatcaaactttttaaagaaagaaaatactttttgactcatggtttaaaggcatcctttccatcttggggaggtcatgggtgctcctggcaacttgcttgaggaagataggtcagaaagcagagtggaccaaccgttcaatgttttacaagcaaaacatacactaagcatggtctgtagctattaaaagcacacaatctgaagggctgtagatgcacagtagtgttttcccagagcatgttcaaaagccctgggttcaatcacaatactgaaaagtaggccaaaaaacattctgaaaatgaaatatttgggtttttttttataacctttagtgactaaataaagacaaatctaagagactaaaaaaaaaaaaaaaaaa

SEQ ID NO: 295 - RNA, 561 nt, synthetic construct
  source 1..561: mol_type = other RNA
aatattcttaatctttgcaccttttcctactttggtgtttgtgaatagaagtaaaaataaatacgactgccacctcacgagaatggacttttccacttgaagacgagagactggagtacagatgctacaccacttttgggcaagtgaagggggagcagccagccacggtggcacaaacctatatcctggtgcttttgaaggtagaagcagggcggtcaggagttaaggccagttgaggctgggctgcagagtgaaagaccatgtctcaagatggtctttctcctccccaaagtagaaaagaaaaccataaaaacaagaggtaaatatattactatttcatcttagaggatagcaggcatcttgaaagggtagagggaccttaaattctcattattgcccccatactacaaactaaaaaacaaacccgaatcaatctcccataaagacagagattcaaataagagtattaaacgttttatttctcaaaccactcacatgcataatgttcttatacacagtgtcaaaataaagagaaatgcatttttatacaaaaaaaaaaaa

SEQ ID NO: 296 - RNA, 46 nt, synthetic construct
  source 1..46: mol_type = other RNA
ctacagcccagccctctaataaagcttcagttgtatttcacccatc

SEQ ID NO: 297 - RNA, 347 nt, synthetic construct
  source 1..347: mol_type = other RNA
aaagttctgctgcacgaagattcctcctgcggcggggggattgctcctcctctggcttggaaacctagcctagaatcagatacactttctttagagtaaagcacaagctgatgagttacgactttgtgaaatggatagccttgaggggaggcgaaaacaggtcccccaaggctatcagatgtcagtgccaatagactgaaacaagtctgtaaagttagcagtcaggggtgttggttggggccggaagaagagacccactgaaactgtagccccttatcaaaacatatccttgcttgaaagaaaaataccaaggacagaaaatgccataaaatcttgactttgcactc

SEQ ID NO: 298 - RNA, 124 nt, synthetic construct
  source 1..124: mol_type = other RNA
cctagagccacatcctgacctctctacacccctgcagcccctcaaccccattatttattcctgccctccccaccaatgaccttgaaataaagacgattttattttcaaaaaaaaaaaaaaaaaa

SEQ ID NO: 299 - RNA, 380 nt, synthetic construct
  source 1..380: mol_type = other RNA
ccaccctaaaatgtcatccttccttctgaattgggttccttccattaaacacaggctggcctggctcgtgcctgatgctacagcaagtccttgactctgtgggttgtgtgtgtgtgtgtgtgtgtgtgtgtgtgtgtgtgtgtgtgtgtgtgtgtgtgtgtgtgtgtgtctgtgtgtgtgtgtgtctttatgccctgagtttgttgtggacttgagatcatagtatgtcttgatatctcctccagccatgcaaataggttgtgggtagaggactgtggctgagaccacagactctggtccaagaaccatctgctctaaaaaaaataaatctgtcatctctggaaaataaagaggacatgctcaatgactcagggtccagc

SEQ ID NO: 300 - RNA, 217 nt, synthetic construct
  source 1..217: mol_type = other RNA
ctgaagggttagaaagtgggggctctgttttctttgctcggttatccgagaagaaagacaaaacggaagatgaaggtgtcacggatcttgtgaactttttaaaactttcaaggtgctattccattgttctttgtactgtagctaaatgtaactgagatgagttactgctttgaaaaaataaagttttacattttttccaccctttaaaaaaaaaaaa

SEQ ID NO: 301 - RNA, 141 nt, synthetic construct
  source 1..141: mol_type = other RNA
gtatccttctcctgtcctgcaacaacatccatatccagccaggtggccctgtctcaagcacctctctggccctctggtggcccttgcttaataaagattctccgagcacattctgagtctctgtgagtgattcaaaaaaaa

SEQ ID NO: 302 - RNA, 1932 nt, synthetic construct
  source 1..1932: mol_type = other RNA
ggacgcctcaggcaccggagccagaccctcccaagaccacccaggccttcctcaaggactctgcctcagacctcagacaggccaccaacgctgttcatcttcatttccccaaggagacttctttctttgtgccttgatgtttgagagttcttcgagcaaacagtggttttgcaatgtctcacaggccctgtttttgtttttgtttttgttttgttttgttttgttctttttttaaatgcaaccaaagtagagtcaacctgctcggcagatgtacttggattctctgaatcgctattctgtttggagagttcctttgggtcttaagcagccagagtacatggaaatgagattatgtcagatctggagaaacaagcaggtgttgggaaatatgtgacttgacatgataagggctgggaatccagaaatcaatagtgagatccatgaaatcaaaccctgaccagtgtgaaaatgtagccttttggacagtaagcctgcaagtctagtgagaactcagagaaagctgaccattctggtctgaagataggcagcgcatcacaggcaagaatatcgaagtcagtagtaggacaggggtcacatcagataccagctcaaattgcactagctatctagaacagttttctccaggtttgcctgagccttgatgcataccatcgccctctgctggtcgcagcagagataagcaagggctgaaaatggaggcaatcctttcccaaggccctgaaagttgtttttcatggtttcaaactgaatttggctcatttgtaactaactgatcacggtgcctggttacactggctgccaagaaggagcgcatgcaatctgattcagtgctctcttcacatcagtttcctgcctccctccctcatctgcggacagcatcctatctcatcaggcttccctgtgtgtcacaaagtagcagccaccaagcaaatatattccttgaattagcacacctgggtgggccatgtgcgcaccaaggaaacaggtgctatagggagcgccaggccaggcttgtctcttaactgtctcgttcttcagtgagagtgggaaagctgtccggagctcccgcgcaggagcctgggtacccacgcagcgagtcaagggagttttcggagccagagagagaaagatgtgaaggctgtggagtaaggctgaaaccagcctcctgccctatagtcccacactgcagggggtgcgactttaaaacagaacttcaagttgttaacactcacaagcattgcattactgtgaaggaagtagccgcatccataacaggatgtgatggtctacagcttttcctttaaaagctgaaaaggtaccatgtgtgctcgctaggcatataatccagatatgctccagagttctgagattcttccatgaaaggttaactagaagctagaatatttttttatatttttgtaacaattggcttttttcatggggggaggggagtagagggttagtatttatagtcctaacaagtccaaaaatttttataagtgtcttcagattataaataaccctccaaattttgcaatgtttacatgttttttttttaagatgacaaatatgcttgatttgctttttaaataaaagtttagctgttctaagagattaacttcaagtaggatggctggttatgatagtttggattttctacaggttctgttgccatgccttttgggtttcagcatcactcgagtcgcagcatgtgggtggggctgtggaaacctggccaggctggacctggtcagccacacctcagagacattgtttccatttggatgtgagcaggcgcaggcctgcatgctctttcctacttagcatcatcagttcttccgcctccttagcatggttctttgtaacagccatgctgggaagctctgaacaataaaatacttccagagtggt

SEQ ID NO: 303 - RNA, 291 nt, synthetic construct
  source 1..291: mol_type = other RNA
agattgtcgaggcatcggtggggccgtcacccttgtttcttttccttttttaaaaaaaaaaaaaaaacagcttttttttttttgagagatacaattctttccccatttaattcatctccaagcaattttacaatagtgtctatcatgttcaccccataacccatactcattaggacttatgatttaagattcctcctaccctgtcttgcttgccgcacctcatgctaatctagtttttgactcaatagatttgcctactctggctgtctcatataaatcgaatgaattatg

SEQ ID NO: 304 - RNA, 257 nt, synthetic construct
  source 1..257: mol_type = other RNA
ctaggtggaaggccgagcaaaacctctgcttactaaagcttactgaatatggggagagggcttagggtgtttggaaaaactgacagtaatcaaactgggacactacactgaaccacagcttcctgtcgcccctcagcccctcccctttttttgtattattgtgggtaaaattttcctgtctgtggacttctggattttgtgacaatagaccatcactgctgtgacctttgttgaaaataaactcgatacttactttg

SEQ ID NO: 305 - RNA, 282 nt, synthetic construct
  source 1..282: mol_type = other RNA
agaatggcctgagcctccagtgttgagtggagacttttcaccaggactccagcatcatcccttcctatccatacagactcccatgccaaggtctgtgatctgctctccacctgtctcacagagaagtgcaatcccgttctctccagcatgttacctaggataactcatcaagaatcaaagactttctttaaatttctctttgccaacacatggaaattctccattgatttctttcctgtcctgttcaataaatgattacacttgcacttaaaaaaaaaaaaa

SEQ ID NO: 306 - RNA, 509 nt, synthetic construct
  source 1..509: mol_type = other RNA
ctccttggatagcccaacccgtcccaagaaggaagctacggcctgtgaagctgttctatggactttcctgctattcttgtgtaagggaagagaatgagataaagagagagtgaagaaagcagagggggaggtaaatgagagaggctgggaaaggggaaacagaaagcagggccgggggaagagtctaagttagagactcacaaagaaactcaagaggggctgggcagtgcagtcacagtcaggcagctgaggggcagggtgtccctgagggaggcgaggctcaggccttgctcccgtctccccgtagctgcctcctgtctgcatgcattcggtctgcagtactacacagtaggtatgcacatgagcacgtaggacacgtgaatgtgccgcatgcatgtgcgtgcctgtgtgtccatcattggcactgttgctcacttgtgcttcctgtgagcaccctgtcttggtttcaattaaatgagaaacatggtcaaaaaaaaaaaaaaaaaaaa

SEQ ID NO: 307 - RNA, 1098 nt, synthetic construct
  source 1..1098: mol_type = other RNA
ccgtggtgactgcctcccaggagctgggtccccagggcctgcactgcctgcatagggggtgaggagggccgcagccacactgcctggaggatatctgagcctgccatgccacctgacacaggctgctggccttcccagaagtctacgcattcattgacactgctgctcctccatcatcaggaagggatggctctgaggtgtctcagcctgacaagcgagcctcgaggagctggaggacggcccaatctgggcagtattgtggaccaccatccctgctgtttagaataggaaatttaatgcttgggacaggagtggggaagctcgtggtgcccgcacccccccagtcagagcctgcaggccttcaaggatctgtgctgagctctgaggccctagatcaacacagctgcctgctgcctcctgcacctccccaggccattccaccctgcaccagagacccacgtgcctgtttgaggattaccctccccaccacggggatttcctacccagctgttctgctaggctcgggagctgaggggaagccactcggggctctcctaggctttcccctaccaagccatcccttctcccagccccaggactgcacttgcaggccatctgttcccttggatgtgtcttctgatgccagcctggcaacttgcatccactagaaaggccatttcagggctcgggttgtcatccctgttccttaggacctgcaactcatgccaagaccacaccatggacaatccactcctctgcctgtaggcccctgacaacttccttcctgctatgagggagacctgcagaactcagaagtcaaggcctgggcagtgtctagtggagagggtaccaagaccagcagagagaagccacctaagtggcctgggggctagcagccattctgagaaatcctgggtcccgagcagcccagggaaacacagcacacatgactgtctcctcgggcctactgcagggaacctggccttcagccagctcctttgtcatcctggactgtagcctacggccaaccataagtgagcctgtatgtttatttaacttttagtaaagtcagtaaaaagcaaaaaaaaaaaaaaaaaa

SEQ ID NO: 308 - RNA, 201 nt, synthetic construct
  source 1..201: mol_type = other RNA
acatctccagaaggaagagtggacaaaaaaatgtgttgactctttggtgtgagccttttggcttaactgtaactgctagtactttaaccacatggtgaagatgtccatgtgagatttctataccttaggaataaaaacttttcaactatttctcttctcctagtctgctttttttttattaaaaaatacttttttccattt

SEQ ID NO: 309 - RNA, 739 nt, synthetic construct
  source 1..739: mol_type = other RNA
tctttccagccccaccctacaagtgtctctctaccaaggtcaatccacaccccagtgatgttagcagaccctccatctttgagtggtcctttcacccttaagccttttgctctggagccatgttctcagcttcagcacaatttacagcttctccaagcatcgccccgtgggatgttttgagacttctctcctcaatggtgacagttggtcaccctgttctgcttcagggtttcagtactgctcagtgttgtttaagagaatcaaaagttcttatggtttggtctgggatcaatagggaaacacaggtagccaactaggaggaaatgtactgaatgctagtacccaagaccttgagcaggaaagtcacccagacacctctgctttcttttgccatctgacctgcagcactgtcaggacatggcctgtggctgtgtgttcaaacacccctcccacaggactcactttgtcccaacaattcagattgcctagaaatacctttctcttacctgtttgttatttatcaatttttcccagtatttttatacggaaaaaattgtattgaagacactttgtatgcagttgataagaggaattcagtataattatggttggtgattatttttataagcacatgccaacgctttactactgtggaaagacaagtgttttaataaaaagatttacattccatgatgtggacgtcatttctttttttttttaacatcatgtgtttggagag

SEQ ID NO: 310 - RNA, 252 nt, synthetic construct
  source 1..252: mol_type = other RNA
caacgtctaggatgtgaagtttgaagatttctgattagctttcatccggtcttcatctctatttatcttagaagtttagtttcccccacctcccctaccttctctaggtggacattaaaccatcgtccaaagtacatgagagtcactgactctgttcacacaactgtatgtcttactgaaggtccctgaaagatgtttgaggcttgggattccaaacttggtttattaaacatatagtcaccatcttcctat

SEQ ID NO: 311 - RNA, 188 nt, synthetic construct
  source 1..188: mol_type = other RNA
gcccatcaccccacctgggtggctggcattcaggaacctaactgaagtcttctctgcaccccctgccaaccccttcccatctacagtgttagtggtcccggtgccacagagaagagcccagttggaagctatacccgatttaattccagaattagtcaaccatcaattagaatccatccacccccctc

SEQ ID NO: 312 - RNA, 254 nt, synthetic construct
  source 1..254: mol_type = other RNA
gcatcctctcaccagactatgccctcctggaggggctgggaatatagcaagaacgaaaagactgtgcaaggcctagagccagcaaagatgctgatgtagccaggccatgccggaaggagcagggtgaagcttcccctctccctacaaatggaaccttgtggaaacaggatgctaaacaccttctgatggagctgttgcctgcaggccactggtctttgggaattttcaataaagtgcttgcgaggaatctccta

SEQ ID NO: 313 - RNA, 243 nt, synthetic construct
  source 1..243: mol_type = other RNA
agccaagactgtgatacttctcctgtaccctgttgacctcagggagtgctgaccctgtctggtgacttagcaccctcctgtccccagcactgctcctttcagctgctggagctcttggcctggacccctgctggtgacaggacaccctctgaacaatcagaagtggctccaagtggagtgagcagtcatgtcccccatgaataaaaattgtgagcagaggtcgcctacaaaaaaaaaaaaaaa

SEQ ID NO: 314 - RNA, 118 nt, synthetic construct
  source 1..118: mol_type = other RNA
agctccggaagtcacaagacacacccttgccttatgaggatcatgctaccactgcatcagtcaggaatgaataaagctactttgattgtgggaaatgccacagaaaaaaaaaaaaaaa

SEQ ID NO: 315 - RNA, 223 nt, synthetic construct
  source 1..223: mol_type = other RNA
aggccagccttgcccctgccccagagcagagctcaagtgacgctactccattctgcatgttgtacattcctagaaacaaacctaacagcgtggatagtttcacagcttaatgctttgcaatgcccaaggtcacttcatcctcatgctataatgccactgtatcaggtaatatatattttgagtaggtgaaggagaaataaacacatctttcctttataaatta

SEQ ID NO: 316 - RNA, 146 nt, synthetic construct
  source 1..146: mol_type = other RNA
gtttctccggctcccagaagcccatgctcaggcaatggcccctaccctaagaccatcccctaatgcagatattgcatttgggtgcagatgtgggggtcgggcaaacggagtaaacaatacagtctgcattctccaaaaaaaaaaaa

SEQ ID NO: 317 - RNA, 216 nt, synthetic construct
  source 1..216: mol_type = other RNA
gcccccatccacacatggaccacgcaaagtgctggacacatcagtcatctccaactggctgaaaggctgaacctcagggctccacccacgtcatggccacgccccctctattacaagagtccgccttgcctgagtcctccctgctgagtaaagctaccctcccaggtccaaaaaaaaaaaaaaaaaaaaaaaaaaaaaaaaaaaaaaaaaaaaaaa

SEQ ID NO: 318 - protein, 4 aa, synthetic construct
GGGS

SEQ ID NO: 319 - protein, 4 aa, synthetic construct
GPGP

SEQ ID NO: 320 - protein, 8 aa, synthetic construct
GGGSGGGS